(19) United States
(12) Patent Application Publication
(10) Pub. No.: US 2026/0124222 A1
(43) Pub. Date: May 7, 2026

(54) ISOTHIAZOLYLCARBOXAMIDE COMPOUNDS AND THEIR USE IN THERAPY

(71) Applicant: HOTSPOT THERAPEUTICS, INC., Boston, MA (US)

(72) Inventors: Yingzhi Bi, Plainsboro, NJ (US); Geraldine Cirillo Harriman, Charlestown, RI (US); Kenneth G. Carson, Plainsboro, NJ (US); Christian Josef Kuper, Hertford (GB); Sebastien Louis Degorce, Saffron Walden (GB); Arwel Lewis, Royston (GB)

(21) Appl. No.: 19/110,203

(22) PCT Filed: Sep. 12, 2023
(86) PCT No.: PCT/US2023/073971

§ 371 Date: Mar. 10, 2025

Related U.S. Application Data
(60) Provisional application No. 63/405,676, filed on Sep. 12, 2022.

(51) Int. Cl. A61K 31/695; A61K 31/351; A61K 31/426; A61K 31/427; A61K 31/437; A61K 31/44; A61K 31/4439; A61K 31/69; C07D 213/53; C07D 277/22; C07D 277/56; C07D 309/04; C07D 401/04; C07D 417/04; C07D 417/14; C07D 471/04; C07F 5/02; C07F 7/18

(52) U.S. Cl. CPC A61K 31/695; A61K 31/351; A61K 31/426; A61K 31/427; A61K 31/437; A61K 31/44; A61K 31/4439; A61K 31/69; C07D 213/53; C07D 277/22; C07D 277/56; C07D 309/04; C07D 401/04; C07D 417/04; C07D 417/14; C07D 471/04; C07F 5/027; C07F 7/1804

(57) ABSTRACT

The invention provides isothiazolylcarboxamide compounds, pharmaceutical compositions, their use for inhibiting mucosa-associated lymphoid tissue lymphoma translocation protein 1 (MALT 1), and their use in the treatment of a disease or condition, such as a proliferative disorder, inflammatory disorder, or autoimmune disorder.

### CROSS-REFERENCE TO RELATED APPLICATIONS

This application claims the benefit of and priority to U.S. Provisional Patent Application Ser. No. 63/405,676, filed Sep. 12, 2022, the contents of which are hereby incorporated by reference in their entirety.

### FIELD OF THE INVENTION

The invention provides isothiazolylcarboxamide compounds, pharmaceutical compositions, their use for inhibiting mucosa-associated lymphoid tissue lymphoma translocation protein 1 (MALT1). and their use in the treatment of a disease or condition, such as a proliferative disorder, inflammatory disorder, or autoimmune disorder.

### BACKGROUND

Cancer continues to be a significant health problem despite the substantial research efforts and scientific advances reported in the literature for treating this disease. Solid tumors, including prostate cancer, breast cancer, and lung cancer remain highly prevalent among the world population. Current treatment options for these cancers are not effective for all patients and/or can have substantial adverse side effects. Moreover, new therapies that achieve an anti-cancer effect through a different mechanism present an opportunity to treat cancers more effectively and/or to treat cancers that have become resistant to currently available medicines.

Inflammatory disorders impact a substantial number of patients and often involve situations where the patient's biological response to a stimulus results in the immune system attacking the body's own cells or tissues. This can lead to abnormal inflammation and result in chronic pain, redness, swelling, stiffness, and/or damage to normal tissues. Current treatment options for these inflammatory disorders are not effective for all patients and/or can have substantial adverse side effects.

Human mucosa-associated lymphoid tissue protein 1 (MALT1) is a key regulator of immune responses and is an immune modulatory target for the treatment of autoimmune and inflammatory diseases. In addition, research indicates that MALT1 inhibition impairs immune suppressive function of regulatory T cells in a tumor microenvironment, implicating MALT1 inhibitors for boosting anti-tumor immunity in the treatment of solid cancers. See, for example, Isabel Hamp et al. in Expert Opinion on Therapeutic Patents (2021) vol. 12, pages 1079-1096.

The present invention addresses the foregoing needs and provides other related advantages.

### SUMMARY

The invention provides isothiazolylcarboxamide compounds, pharmaceutical compositions, their use for inhibiting mucosa-associated lymphoid tissue lymphoma translocation protein 1 (MALT1), and their use in the treatment of a disease or condition, such as a proliferative disorder, inflammatory disorder, or autoimmune disorder. In particular, one aspect of the invention provides a collection of isothiazolylcarboxamide compounds, such as a compound represented by Formula I.

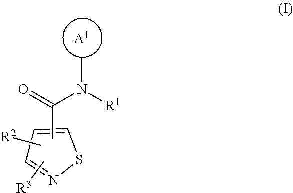

or a pharmaceutically acceptable salt thereof, where the variables are as defined in the detailed description. Further description of additional collections of isothiazolylcarboxamide compounds are described in the detailed description. The compounds may be part of a pharmaceutical composition comprising a pharmaceutically acceptable carrier.

Another aspect of the invention provides a method of treating a disease or condition mediated by MALT1 in a subject. The method comprises administering a therapeutically effective amount of a compound described herein, such as a compound of Formula I or I-1, or other compound in Section I, to a subject in need thereof to treat the disease or condition, as further described in the detailed description.

Another aspect of the invention provides a method of inhibiting the activity of MALT1. The method comprises contacting a MALT1 with an effective amount of a compound described herein, such as a compound of Formula I or I-1, or other compound in Section I, to inhibit the activity of said MALT1, as further described in the detailed description.

### DETAILED DESCRIPTION

The invention provides isothiazolylcarboxamide compounds, pharmaceutical compositions, their use for inhibiting mucosa-associated lymphoid tissue lymphoma translocation protein 1 (MALT1), and their use in the treatment of a disease or condition, such as a proliferative disorder, inflammatory disorder, or autoimmune disorder. The practice of the present invention employs, unless otherwise indicated, conventional techniques of organic chemistry, pharmacology, molecular biology (including recombinant techniques), cell biology, biochemistry, and immunology. Such techniques are explained in the literature, such as in Comprehensive Organic Synthesis (B. M. Trost & I. Fleming, eds., 1991-1992); Handbook of Experimental Immunology (D. M. Weir & C. C. Blackwell, eds.); Current Protocols in Molecular Biology (F. M. Ausubel et al., eds., 1987, and periodic updates); and Current Protocols in Immunology” (J. E. Coligan et al., eds., 1991), each of which is herein incorporated by reference in its entirety.

Various aspects of the invention are set forth below in sections; however, aspects of the invention described in one particular section are not to be limited to any particular section. Further, when a variable is not accompanied by a definition, the previous definition of the variable controls.

### Definitions

Compounds of the present invention include those described generally herein, and are further illustrated by the classes, subclasses, and species disclosed herein. As used herein, the following definitions shall apply unless otherwise indicated. These definitions apply regardless of whether a term is used by itself or in combination with other terms, unless otherwise indicated. Hence, the definition of “alkyl” applies to “alkyl” as well as the “alkyl” portions of “—O-alkyl” etc. For purposes of this invention, the chemical elements are identified in accordance with the Periodic Table of the Elements, CAS version, Handbook of Chemistry and Physics, 75^{th} Ed. Additionally, general principles of organic chemistry are described in Organic Chemistry, Thomas Sorrell, University Science Books, Sausalito: 1999, and March's Advanced Organic Chemistry, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e., carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In some embodiments, a bicyclic group has 7-12 ring members and 0-4 heteroatoms independently selected from nitrogen, oxygen, or sulfur. As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

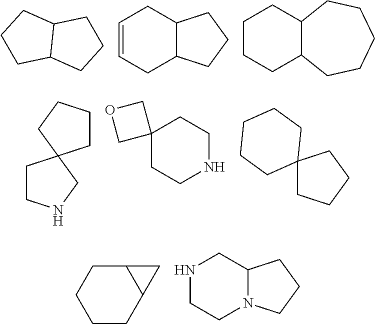

Exemplary bridged bicyclics include:

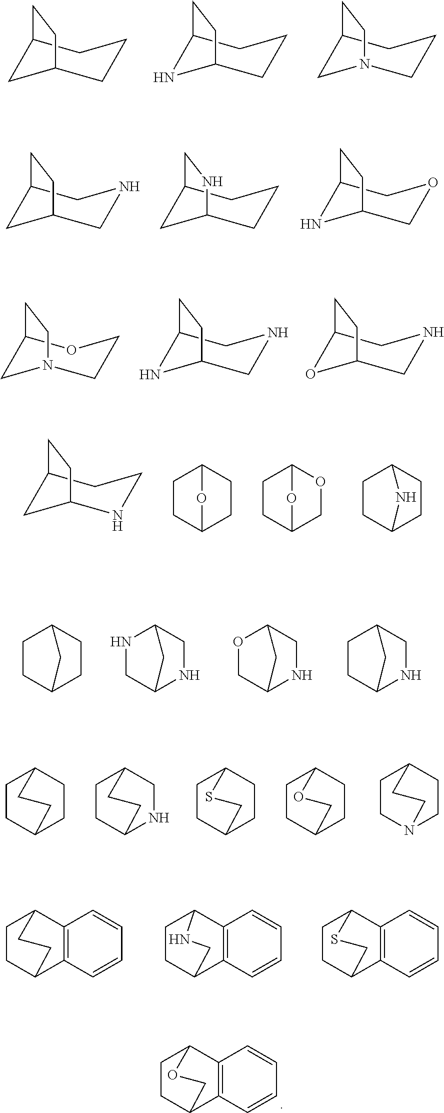

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including any oxidized form of nitrogen, sulfur, phosphorus, or silicon; the quaternized form of any basic nitrogen; or a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “—(C₀ alkylene)-” refers to a bond. Accordingly, the term “—(C₀₋₃ alkylene)-” encompasses a bond (i.e., C₀) and a —(C₁₋₃ alkylene)- group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” or “halo” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but is not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like. The term “phenylene” refers to a multivalent phenyl group having the appropriate number of open valences to account for groups attached to it. For example, “phenylene” is a bivalent phenyl group when it has two groups attached to it (e.g.,

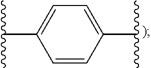 [SMILES: *c1ccc(*)cc1]

“phenylene” is a trivalent phenyl group when it has three groups attached to it (e.g.,

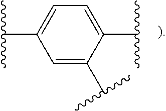 [SMILES: *c1ccc(*)c(*)c1]

The term “arylene” refers to a bivalent aryl group.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

The term “heteroarylene” refers to a multivalent heteroaryl group having the appropriate number of open valences to account for groups attached to it. For example, “heteroarylene” is a bivalent heteroaryl group when it has two groups attached to it; “heteroarylene” is a trivalent heteroaryl group when it has three groups attached to it. The term “pyridinylene” refers to a multivalent pyridine radical having the appropriate number of open valences to account for groups attached to it. For example, “pyridinylene” is a bivalent pyridine radical when it has two groups attached to it (e.g.,

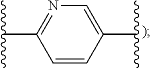 [SMILES: *c1ccc(*)nc1]

“pyridinylene” is a trivalent pyridine radical when it has three groups attached to it (e.g.,

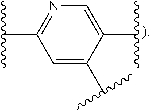 [SMILES: *c1cc(*)c(*)cn1]

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted. The term “oxo-heterocyclyl” refers to a heterocyclyl substituted by an oxo group. The term “heterocyclylene” refers to a multivalent heterocyclyl group having the appropriate number of open valences to account for groups attached to it. For example, “heterocyclylene” is a bivalent heterocyclyl group when it has two groups attached to it; “heterocyclylene” is a trivalent heterocyclyl group when it has three groups attached to it.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Each optional substituent on a substitutable carbon is a monovalent substituent independently selected from halogen; —(CH₂)₀₋₄R^{○}; —(CH₂)₀₋₄OR^{○}; —O(CH₂)₀₋₄R^{○}, —O—(CH₂)₀₋₄C(O)OR^{○}; —(CH₂)₀₋₄CH(OR^{○})₂; —(CH₂)₀₋₄SR^{○}; —(CH₂)₀₋₄Ph, which may be substituted with R^{○}; —(CH₂)₀₋₄O(CH₂)₀₋₁Ph which may be substituted with R^{○}; —CH═CHPh, which may be substituted with R^{○}; —(CH₂)₀₋₄₀(CH₂)₀₋₁-pyridyl which may be substituted with R^{○}; —NO₂; —CN; —N₃; —(CH₂)₀₋₄N(R^{○})₂; —(CH₂)₀₋₄N(R^{○})C(O)R^{○}; —N(R^{○})C(S)R^{○}; —(CH₂)₀₋₄N(R^{○})C(O)NR^{○}₂; —N(R^{○})C(S)NR^{○}₂; —(CH₂)₀₋₄N(R^{○})C(O)OR^{○}; —(R^{○})N(R^{○})C(O)R^{○}; —N(R^{○})N(R^{○})C(O)NR^{○}₂; —N(R^{○})N(R^{○})C(O)OR^{○}; —(CH₂)₀₋₄C(O)R^{○}; —C(S)R^{○}; —(CH₂)₀₋₄C(O)OR^{○}; —(CH₂)₀₋₄C(O)SR^{○}; —(CH₂)₀₋₄C(O)OSiR^{○}₃; —(CH₂)₀₋₄OC(O)R^{○}; —OC(O)(CH₂)₀₋₄SR—, SC(S)SR^{○}; —(CH₂)₀₋₄SC(O)R^{○}; —(CH₂)₀₋₄C(O)NR^{○}₂; —C(S)NR^{○}₂; —C(S)SR^{○}; —SC(S)SR^{○}, —(CH₂)₀₋₄OC(O)NR^{○}₂; —C(O)N(OR^{○})R^{○}; —C(O)C(O)R^{○}; —C(O)CH₂C(O)R^{○}; —C(NOR^{○})R^{○}; —(CH₂)₀₋₄SSR^{○}; —(CH₂)₀₋₄S(O)₂R^{○}; —(CH₂)₀₋₄S(O)₂OR^{○}; —(CH₂)₀₋₄OS(O)₂R^{○}; —S(O)₂NR^{○}₂; —S(O)(NR^{○})R^{○}; —S(O)₂N═C(NR^{○}₂)₂; —(CH₂)₀₋₄S(O)R^{○}; —N(R^{○})S(O)₂NR^{○}₂; —N(R^{○})S(O)₂R^{○}; —N(OR^{○})R^{○}; —C(NH)NR^{○}₂; —P(O)₂R^{○}; —P(O)R^{○}₂; —OP(O)R^{○}₂; —OP(O)(OR^{○})₂; SiR^{○}₃; —(C₁₋₄ straight or branched alkylene)O—N(R^{○})₂; or —(C₁₋₄ straight or branched alkylene)C(O)O—N(R^{○})₂.

Each R^{○} is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{○}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted by a divalent substituent on a saturated carbon atom of R^{○} selected from ═O and ═S; or each R^{○} is optionally substituted with a monovalent substituent independently selected from halogen, —(CH₂)₀₋₂R^{•}, -(haloR^{•}), —(CH₂)₀₋₂OH, —(CH₂)₀₋₂OR′, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•}.

Each R^{○} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{○} is unsubstituted or where preceded by halo is substituted only with one or more halogens; or wherein an optional substituent on a saturated carbon is a divalent substituent independently selected from ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, or a divalent substituent bound to vicinal substitutable carbons of an “optionally substituted” group is —O(CR*₂)₂₋₃O—, wherein each independent occurrence of R* is selected from hydrogen, C₁₋₆ aliphatic or an unsubstituted 5-6-membered saturated, partially unsaturated, or aryl ring having 0-4 heteroatoms independently selected from nitrogen, oxygen, or sulfur.

When R* is C₁₋₆ aliphatic, R* is optionally substituted with halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{○} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{○} is unsubstituted or where preceded by halo is substituted only with one or more halogens.

An optional substituent on a substitutable nitrogen is independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}2, or —N(R^{†})S(O)₂R^{†}; wherein each R^{†} is independently hydrogen, C₁₋₆ aliphatic, unsubstituted —OPh, or an unsubstituted 5-6-membered saturated, partially unsaturated, or aryl ring having 0-4 heteroatoms independently selected from nitrogen, oxygen, or sulfur, or, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur; wherein when R^{†} is C₁₋₆ aliphatic, R^{†} is optionally substituted with halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{○} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{○} is unsubstituted or where preceded by halo is substituted only with one or more halogens.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Further, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-V C H; S. Berge et al., Journal of Pharmaceutical Sciences 1977, 66(1), 1-19; P. Gould, International J. of Pharmaceutics 1986, 33, 201-217; Anderson et al., The Practice of Medicinal Chemistry (1996), Academic Press, New York; and in The Orange Book (Food & Drug Administration, Washington, D.C. on their website). These disclosures are incorporated herein by reference.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. The invention includes compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

Diastereomeric mixtures can be separated into their individual diastereomers on the basis of their physical chemical differences by methods known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Alternatively, a particular enantiomer of a compound of the present invention may be prepared by asymmetric synthesis. Still further, where the molecule contains a basic functional group (such as amino) or an acidic functional group (such as carboxylic acid) diastereomeric salts are formed with an appropriate optically-active acid or base, followed by resolution of the diastereomers thus formed by fractional crystallization or chromatographic means known in the art, and subsequent recovery of the pure enantiomers.

Individual stereoisomers of the compounds of the invention may, for example, be substantially free of other isomers, or may be admixed, for example, as racemates or with all other, or other selected, stereoisomers. Chiral center(s) in a compound of the present invention can have the S or R configuration as defined by the IUPAC 1974 Recommendations. Further, to the extent a compound described herein may exist as an atropisomer (e.g., substituted biaryls), all forms of such atropisomer are considered part of this invention.

Chemical names, common names, and chemical structures may be used interchangeably to describe the same structure. If a chemical compound is referred to using both a chemical structure and a chemical name, and an ambiguity exists between the structure and the name, the structure predominates. It should also be noted that any carbon as well as heteroatom with unsatisfied valences in the text, schemes, examples and tables herein is assumed to have the sufficient number of hydrogen atom(s) to satisfy the valences.

Unless specified otherwise, the term “about” refers to within ±10% of the stated value. The invention encompasses embodiments where the value is within ±9%, ±8%, ±7%, ±6%, ±5%, ±4%, ±3%, ±2%, or 1% of the stated value.

The terms “a” and “an” as used herein mean “one or more” and include the plural unless the context is inappropriate.

The term “alkyl” refers to a saturated straight or branched hydrocarbon, such as a straight or branched group of 1-12, 1-10, or 1-6 carbon atoms, referred to herein as C₁-C₂ alkyl, C₁-C₁₀ alkyl, and C₁-C₆ alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₃-C₆ cycloalkyl,” derived from a cycloalkane. Exemplary cycloalkyl groups include cyclohexyl, cyclopentyl, cyclobutyl, and cyclopropyl. The term “cycloalkylene” refers to a bivalent cycloalkyl group.

The term “haloalkyl” refers to an alkyl group that is substituted with at least one halogen. Exemplary haloalkyl groups include —CH₂F, —CHF₂, —CF₃, —CH₂CF₃, —CF₂CF₃, and the like. The term “haloalkylene” refers to a bivalent haloalkyl group.

The terms “alkenyl” and “alkynyl” are art-recognized and refer to unsaturated aliphatic groups analogous in length and possible substitution to the alkyls described above, but that contain at least one double or triple bond, respectively.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. The term “haloalkoxyl” refers to an alkoxyl group that is substituted with at least one halogen. Exemplary haloalkoxyl groups include —OCH₂F, —OCHF₂, —OCF₃, —OCH₂CF₃, —OCF₂CF₃, and the like. The term “hydroxyalkoxyl” refers to an alkoxyl group that is substituted with at least one hydroxyl. Exemplary hydroxyalkoxyl groups include —OCH₂CH₂OH, —OCH₂C(H)(OH)CH₂CH₂OH, and the like. The term “alkoxylene” refers to a bivalent alkoxyl group.

The term “oxo” is art-recognized and refers to a “═O” substituent. For example, a cyclopentane substituted with an oxo group is cyclopentanone.

The symbol “ [symbol] ” indicates a point of attachment.

When any substituent or variable occurs more than one time in any constituent or the compound of the invention, its definition on each occurrence is independent of its definition at every other occurrence, unless otherwise indicated.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

As used herein, the terms “subject” and “patient” are used interchangeably and refer to organisms to be treated by the methods of the present invention. Such organisms preferably include, but are not limited to, mammals (e.g., murines, simians, equines, bovines, porcines, canines, felines, and the like), and, most preferably, include humans.

As used herein, the term “compound” refers to a quantity of molecules that is sufficient to be weighed, tested for its structural identity, and to have a demonstrable use (e.g., a quantity that can be shown to be active in an assay, an in vitro test, or in vivo test, or a quantity that can be administered to a patient and provide a therapeutic benefit).

The term “IC₅₀” is art-recognized and refers to the concentration of a compound that is required to achieve 50% inhibition of the target.

As used herein, the term “effective amount” refers to the amount of a compound sufficient to effect beneficial or desired results (e.g., a therapeutic, ameliorative, inhibitory, or preventative result). An effective amount can be administered in one or more administrations, applications, or dosages and is not intended to be limited to a particular formulation or administration route.

As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate-buffered saline solution, water, emulsions (e.g., such as oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified.

### I. Isothiazolylcarboxamide Compounds

The invention provides isothiazolylcarboxamide compounds. The compounds may be used in the pharmaceutical compositions and therapeutic methods described herein. Exemplary compounds are described in the following sections, along with exemplary procedures for making the compounds.

One aspect of the invention provides a compound represented by Formula I:

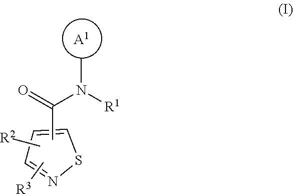

or a pharmaceutically acceptable salt thereof, wherein:
A¹ is phenyl, a 6 membered heteroaryl containing 1 or 2 nitrogen atoms, a 9-10 membered bicyclic heteroaryl containing 1, 2, or 3 heteroatoms independently selected from nitrogen and oxygen,

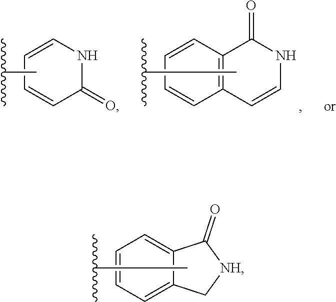

wherein the phenyl, heteroaryl,

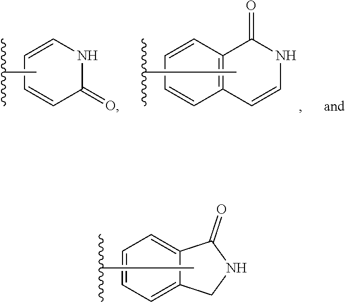

and are substituted with m occurrences of R⁵ and n occurrences of R⁶;
A² represents independently for each occurrence a 4-6 membered saturated heterocyclyl containing 1 or 2 heteroatoms independently selected from oxygen, nitrogen, and sulfur, wherein the heterocyclyl is substituted with y occurrences of R⁷;
R¹ is hydrogen or C₁₋₄ alkyl;
R² is C₁₋₆ haloalkyl, C₁₋₆ alkyl, C₃₋₇ cycloalkyl, C₁₋₆ alkoxyl, cyano, C₂₋₄ alkenyl, halogen, hydrogen, oxetanyl, tetrahydrofuranyl, or —N(R¹²)(R¹³), wherein the oxetanyl and tetrahydrofuranyl are substituted with 0 or 1 hydroxyl groups;
R³ is one of the following:
(a) phenyl, a 5-6 membered heteroaryl containing 1, 2, or 3 heteroatoms independently selected from oxygen, nitrogen, and sulfur, a 9-10 membered bicyclic heteroaryl containing 1, 2, or 3 heteroatoms independently selected from oxygen, nitrogen, and sulfur, a 3-10 membered monocyclic or bicyclic saturated or partially unsaturated heterocyclyl containing 1, 2, or 3 heteroatoms independently selected from oxygen, nitrogen, and sulfur,

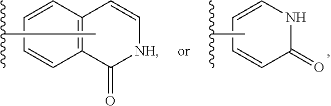

wherein the phenyl, heteroaryl, heterocyclyl,

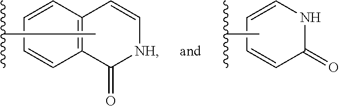

are substituted with t occurrences of R⁴; or
(b) C₂₋₆ alkyl, hydroxyl, or —N(R⁹)(R¹⁰);
R⁴ represents independently for each occurrence halo, hydroxyl, C₁₋₆ alkyl, C₁₋₆ haloalkyl, C₁₋₆hydroxyalkyl, C₁₋₆ alkoxyl, C₃₋₇ cycloalkyl, —C(O)R¹¹, —C(O)N(R⁹)(R¹⁰), cyano, —N(R¹²)(R¹³), or —N(R⁹)C(O)R¹⁰;
R⁵ is a 5-6 membered heteroaryl containing 1, 2, or 3 heteroatoms independently selected from oxygen, nitrogen, and sulfur, or a 3-7 membered saturated heterocyclyl containing 1, 2, or 3 heteroatoms independently selected from oxygen, nitrogen, and sulfur, wherein the heteroaryl and heterocyclyl are substituted with q occurrences of R⁷;
R⁶ represents independently for each occurrence halo, hydroxyl, C₁₋₆ alkyl, C₁₋₆ haloalkyl, C₁₋₆hydroxyalkyl, C₁₋₆ alkoxyl, C₃₋₇ cycloalkyl, cyano, —O—C₃₋₇ cycloalkyl, —N(R⁹)(R¹⁰), —(C₀₋₄ alkylene)-C(O)R⁸, —C(O)N(R⁹)(R¹⁰), or —N(R⁹)C(O)R¹⁰;
R⁷ represents independently for each occurrence halo, hydroxyl, C₁₋₆ alkyl, C₁₋₆ haloalkyl, C₁₋₆ alkoxyl, or C₃₋₇ cycloalkyl;
R⁸ is —OH, —O—(C₁₋₆ alkyl), —O—C₃₋₇ cycloalkyl, or A²;
R⁹ and R¹⁰ are independently hydrogen, C₁₋₆ alkyl, C₃₋₇ cycloalkyl, C₂₋₄ hydroxyalkyl, or —(C₂₋₆ alkylene)-(C₁₋₆ alkoxyl), or R⁹ and R¹⁰ are taken together with the nitrogen atom to which they are attached to form a 3-7 membered heterocyclic ring containing 1 nitrogen atom, wherein the heterocyclic ring is substituted with 0 or 1 groups independently selected from hydroxyl, halo, and C₁₋₆ alkyl;
R¹¹ represents independently for each occurrence C₁₋₆ alkyl or (C₀₋₅ alkylene)-C₃₋₇ cycloalkyl;
R¹² and R¹³ are independently hydrogen, C₁₋₆ alkyl, or C₃₋₅ cycloalkyl, or R¹² and R¹³ are taken together with the nitrogen atom to which they are attached to form a 3-7 membered heterocyclic ring containing 1 nitrogen atom;
m is 0 or 1; and
n, q, t, and y are independently 0, 1, or 2;
provided that when R² is hydrogen and m is 1, then R⁵ is 1,2,3-triazolyl substituted with q occurrences of R⁷.

The definitions of variables in Formula I above encompass multiple chemical groups. The application contemplates embodiments where, for example, (i) the definition of a variable is a single chemical group selected from those chemical groups set forth above, (ii) the definition of a variable is a collection of two or more of the chemical groups selected from those set forth above, and (iii) the compound is defined by a combination of variables in which the variables are defined by (i) or (ii).

In certain embodiments, the compound is a compound of Formula I.

As defined generally above, A¹ is phenyl, a 6 membered heteroaryl containing 1 or 2 nitrogen atoms, a 9-10 membered bicyclic heteroaryl containing 1, 2, or 3 heteroatoms independently selected from nitrogen and oxygen,

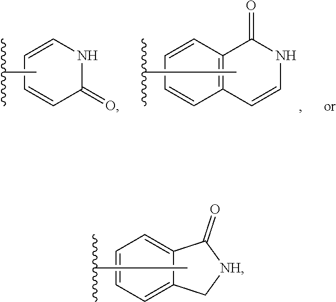

wherein the phenyl, heteroaryl,

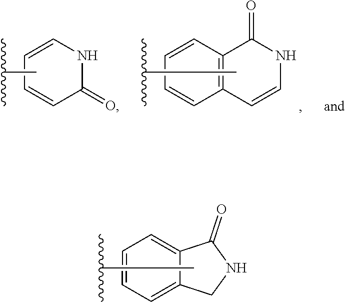

are substituted with m occurrences of R⁵ and n occurrences of R⁶. In certain embodiments, A¹ is phenyl substituted with m occurrences of R⁵ and n occurrences of R⁶. In certain embodiments, A¹ is a 6 membered heteroaryl containing 1 or 2 nitrogen atoms, wherein the heteroaryl is substituted with m occurrences of R⁵ and n occurrences of R⁶. In certain embodiments, A¹ is a pyridinyl substituted with m occurrences of R⁵ and n occurrences of R⁶. In certain embodiments, A¹ is a 9-10 membered bicyclic heteroaryl containing 1, 2, or 3 heteroatoms independently selected from nitrogen and oxygen, wherein the heteroaryl is substituted with m occurrences of R⁵ and n occurrences of R⁶. In certain embodiments, A¹ is

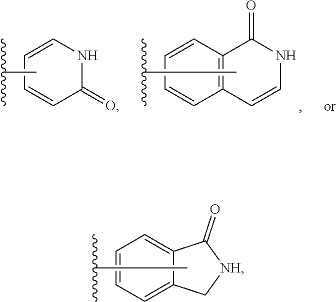

each of which is substituted with m occurrences of R⁵ and n occurrences of R⁶. In certain embodiments, A¹ is a 6 membered heteroaryl containing 1 nitrogen atom, wherein the heteroaryl is substituted with m occurrences of R⁵ and n occurrences of R⁶. In certain embodiments, A¹ is a 6 membered heteroaryl containing 2 nitrogen atoms, wherein the heteroaryl is substituted with m occurrences of R⁵ and n occurrences of R⁶. In certain embodiments, A¹ is 9 membered bicyclic heteroaryl containing 1, 2, or 3 heteroatoms independently selected from nitrogen and oxygen, wherein the heteroaryl is substituted with m occurrences of R⁵ and n occurrences of R⁶. In certain embodiments. A¹ is 9 membered bicyclic heteroaryl containing 1 heteroatom selected from nitrogen and oxygen, wherein the heteroaryl is substituted with m occurrences of R⁵ and n occurrences of R⁶. In certain embodiments, A¹ is 9 membered bicyclic heteroaryl containing 2 heteroatoms independently selected from nitrogen and oxygen, wherein the heteroaryl is substituted with m occurrences of R⁵ and n occurrences of R⁶. In certain embodiments, A¹ is 9 membered bicyclic heteroaryl containing 3 heteroatoms independently selected from nitrogen and oxygen, wherein the heteroaryl is substituted with m occurrences of R⁵ and n occurrences of R⁶. In certain embodiments, A¹ is 10 membered bicyclic heteroaryl containing 1, 2, or 3 heteroatoms independently selected from nitrogen and oxygen, wherein the heteroaryl is substituted with m occurrences of R⁵ and n occurrences of R⁶. In certain embodiments, A¹ is 10 membered bicyclic heteroaryl containing 1 heteroatom selected from nitrogen and oxygen, wherein the heteroaryl is substituted with m occurrences of R⁵ and n occurrences of R⁶. In certain embodiments, A¹ is 10 membered bicyclic heteroaryl containing 2 heteroatoms independently selected from nitrogen and oxygen, wherein the heteroaryl is substituted with m occurrences of R⁵ and n occurrences of R⁶. In certain embodiments, A¹ is 10 membered bicyclic heteroaryl containing 3 heteroatoms independently selected from nitrogen and oxygen, wherein the heteroaryl is substituted with m occurrences of R⁵ and n occurrences of R⁶. In certain embodiments, A¹ is

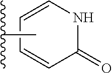

substituted with m occurrences of R⁵ and n occurrences of R⁶. In certain embodiments, A¹ is

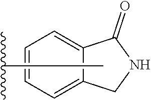

substituted with m occurrences of R⁵ and n occurrences of R⁶. In certain embodiments, A¹ is

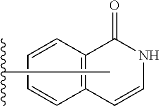

substituted with m occurrences of R⁵ and n occurrences of R⁶. In certain embodiments, A¹ is selected from the groups depicted in the compounds in Table 1 below.

As defined generally above, A² represents independently for each occurrence a 4-6 membered saturated heterocyclyl containing 1 or 2 heteroatoms independently selected from oxygen, nitrogen, and sulfur, wherein the heterocyclyl is substituted with y occurrences of R⁷. In certain embodiments, A² is azetidinyl substituted by hydroxyl. In certain embodiments, A² is a 4 membered saturated heterocyclyl containing 1 or 2 heteroatoms independently selected from oxygen, nitrogen, and sulfur, wherein the heterocyclyl is substituted with y occurrences of R⁷. In certain embodiments, A² is a 4 membered saturated heterocyclyl containing 1 heteroatom selected from oxygen, nitrogen, and sulfur, wherein the heterocyclyl is substituted with y occurrences of R⁷. In certain embodiments, A² is a 4 membered saturated heterocyclyl containing 2 heteroatoms independently selected from oxygen, nitrogen, and sulfur, wherein the heterocyclyl is substituted with y occurrences of R⁷. In certain embodiments, A² is a 5 membered saturated heterocyclyl containing 1 or 2 heteroatoms independently selected from oxygen, nitrogen, and sulfur, wherein the heterocyclyl is substituted with y occurrences of R⁷. In certain embodiments, A² is a 5 membered saturated heterocyclyl containing 1 heteroatom selected from oxygen, nitrogen, and sulfur, wherein the heterocyclyl is substituted with y occurrences of R⁷. In certain embodiments, A² is a 5 membered saturated heterocyclyl containing 2 heteroatoms independently selected from oxygen, nitrogen, and sulfur, wherein the heterocyclyl is substituted with y occurrences of R⁷. In certain embodiments, A² is a 6 membered saturated heterocyclyl containing 1 or 2 heteroatoms independently selected from oxygen, nitrogen, and sulfur, wherein the heterocyclyl is substituted with y occurrences of R⁷. In certain embodiments, A² is a 6 membered saturated heterocyclyl containing 1 heteroatom selected from oxygen, nitrogen, and sulfur, wherein the heterocyclyl is substituted with y occurrences of R⁷. In certain embodiments, A² is a 6 membered saturated heterocyclyl containing 2 heteroatoms independently selected from oxygen, nitrogen, and sulfur, wherein the heterocyclyl is substituted with y occurrences of R⁷. In certain embodiments, A² is selected from the groups depicted in the compounds in Table 1 below.

As defined generally above, R¹ is hydrogen or C₁₋₄ alkyl. In certain embodiments, R¹ is hydrogen. In certain embodiments, R¹ is C₁₋₄ alkyl. In certain embodiments, R¹ is C₁ alkyl. In certain embodiments, R¹ is C₂ alkyl. In certain embodiments, R¹ is C₃ alkyl. In certain embodiments, R¹ is C₄ alkyl. In certain embodiments, R¹ is selected from the groups depicted in the compounds in Table 1 below.

As defined generally above, R² is C₁₋₆ haloalkyl, C₁₋₆ alkyl, C₃₋₇ cycloalkyl, C₁₋₆ alkoxyl, cyano, C₂₋₄ alkenyl, halogen, hydrogen, oxetanyl, tetrahydrofuranyl, or —N(R¹²)(R¹³), wherein the oxetanyl and tetrahydrofuranyl are substituted with 0 or 1 hydroxyl groups. In certain embodiments, R² is C₁₋₆haloalkyl. In certain embodiments, R² is —CF₃. In certain embodiments, R² is C₃₋₇ cycloalkyl. In certain embodiments, R² is cyclopropyl. In certain embodiments, R² is C₁₋₆ alkyl or C₂₋₄ alkenyl. In certain embodiments, R² is halogen, C₁₋₆ alkoxyl, or cyano. In certain embodiments, R² is C₁₋₆ alkyl. In certain embodiments, R² is C₁₋₆ alkoxyl. In certain embodiments, R² is cyano. In certain embodiments, R² is C₂₋₄ alkenyl. In certain embodiments, R² is halogen. In certain embodiments, R² is hydrogen. In certain embodiments, R² is oxetanyl substituted with 0 or 1 hydroxyl groups. In certain embodiments, R² is oxetanyl. In certain embodiments, R² is oxetanyl substituted with 1 hydroxyl group. In certain embodiments, R² is tetrahydrofuranyl substituted with 0 or 1 hydroxyl group. In certain embodiments, R² is tetrahydrofuranyl. In certain embodiments, R² is tetrahydrofuranyl substituted with 1 hydroxyl group. In certain embodiments, R² is —N(R¹²)(R¹³). In certain embodiments, R² is selected from the groups depicted in the compounds in Table 1 below.

As defined generally above, R³ is one of the following: (a) phenyl, a 5-6 membered heteroaryl containing 1, 2, or 3 heteroatoms independently selected from oxygen, nitrogen, and sulfur, a 9-10 membered bicyclic heteroaryl containing 1, 2, or 3 heteroatoms independently selected from oxygen, nitrogen, and sulfur, a 3-10 membered monocyclic or bicyclic saturated or partially unsaturated heterocyclyl containing 1, 2, or 3 heteroatoms independently selected from oxygen, nitrogen, and sulfur,

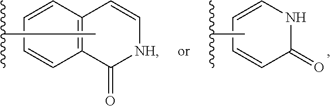

wherein the phenyl, heteroaryl, heterocyclyl,

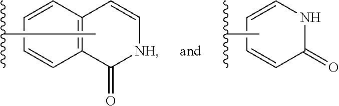

and are substituted with t occurrences of R⁴; or (b) C₂₋₆ alkyl, hydroxyl, or —N(R⁹)(R¹⁰). In certain embodiments, R³ is phenyl substituted with t occurrences of R⁴. In certain embodiments, R³ is a 5-6 membered heteroaryl containing 1, 2, or 3 heteroatoms independently selected from oxygen, nitrogen, and sulfur, wherein the heteroaryl is substituted with t occurrences of R⁴. In certain embodiments, R³ is a 9-10 membered bicyclic heteroaryl containing 1, 2, or 3 heteroatoms independently selected from oxygen, nitrogen, and sulfur, wherein the heteroaryl is substituted with t occurrences of R⁴. In certain embodiments, R³ is a 3-10 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁴. In certain embodiments, R³ is tetrahydropyranyl, morpholinyl, piperidinyl, or piperazinyl, each of which is substituted with t occurrences of R⁴. In certain embodiments, R³ is

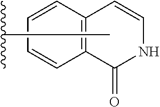

substituted with t occurrences of R⁴. In certain embodiments, R³ is

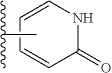

substituted with t occurrences of R⁴. In certain embodiments, R³ is C₃₋₄ alkyl or hydroxyl. In certain embodiments, R³ is —N(R⁹)(R¹⁰).

In certain embodiments, R³ is a 5 membered heteroaryl containing 1, 2, or 3 heteroatoms independently selected from oxygen, nitrogen, and sulfur, wherein the heteroaryl is substituted with t occurrences of R⁴. In certain embodiments, R³ is a 5 membered heteroaryl containing 1 heteroatom selected from oxygen, nitrogen, and sulfur, wherein the heteroaryl is substituted with t occurrences of R⁴. In certain embodiments, R³ is a 5 membered heteroaryl containing 2 heteroatoms independently selected from oxygen, nitrogen, and sulfur, wherein the heteroaryl is substituted with t occurrences of R⁴. In certain embodiments, R³ is a 5 membered heteroaryl containing 3 heteroatoms independently selected from oxygen, nitrogen, and sulfur, wherein the heteroaryl is substituted with t occurrences of R⁴. In certain embodiments, R³ is a 6 membered heteroaryl containing 1, 2, or 3 heteroatoms independently selected from oxygen, nitrogen, and sulfur, wherein the heteroaryl is substituted with t occurrences of R⁴. In certain embodiments, R³ is a 6 membered heteroaryl containing 1 heteroatom selected from oxygen, nitrogen, and sulfur, wherein the heteroaryl is substituted with t occurrences of R⁴. In certain embodiments, R³ is a 6 membered heteroaryl containing 2 heteroatoms independently selected from oxygen, nitrogen, and sulfur, wherein the heteroaryl is substituted with t occurrences of R⁴. In certain embodiments, R³ is a 6 membered heteroaryl containing 3 heteroatoms independently selected from oxygen, nitrogen, and sulfur, wherein the heteroaryl is substituted with t occurrences of R⁴.

In certain embodiments, R³ is a 9 membered bicyclic heteroaryl containing 1, 2, or 3 heteroatoms independently selected from oxygen, nitrogen, and sulfur, wherein the heteroaryl is substituted with t occurrences of R⁴. In certain embodiments, R³ is a 9 membered bicyclic heteroaryl containing 1 heteroatom selected from oxygen, nitrogen, and sulfur, wherein the heteroaryl is substituted with t occurrences of R⁴. In certain embodiments, R³ is a 9 membered bicyclic heteroaryl containing 2 heteroatoms independently selected from oxygen, nitrogen, and sulfur, wherein the heteroaryl is substituted with t occurrences of R⁴. In certain embodiments, R³ is a 9 membered bicyclic heteroaryl containing 3 heteroatoms independently selected from oxygen, nitrogen, and sulfur, wherein the heteroaryl is substituted with t occurrences of R⁴. In certain embodiments, R³ is a 10 membered bicyclic heteroaryl containing 1, 2, or 3 heteroatoms independently selected from oxygen, nitrogen, and sulfur, wherein the heteroaryl is substituted with t occurrences of R⁴. In certain embodiments, R³ is a 10 membered bicyclic heteroaryl containing 1 heteroatom selected from oxygen, nitrogen, and sulfur, wherein the heteroaryl is substituted with t occurrences of R⁴. In certain embodiments, R³ is a 10 membered bicyclic heteroaryl containing 2 heteroatoms independently selected from oxygen, nitrogen, and sulfur, wherein the heteroaryl is substituted with t occurrences of R⁴. In certain embodiments, R³ is a 10 membered bicyclic heteroaryl containing 3 heteroatoms independently selected from oxygen, nitrogen, and sulfur, wherein the heteroaryl is substituted with t occurrences of R⁴.

In certain embodiments, R³ is a 3 membered monocyclic saturated heterocyclyl containing 1 heteroatom selected from oxygen, nitrogen, and sulfur, wherein the heterocyclyl is substituted with t occurrences of R⁴. In certain embodiments, R³ is a 4 membered monocyclic saturated heterocyclyl containing 1 heteroatom selected from oxygen, nitrogen, and sulfur, wherein the heterocyclyl is substituted with t occurrences of R⁴. In certain embodiments, R³ is a 4 membered monocyclic saturated heterocyclyl containing 2 heteroatoms independently selected from oxygen, nitrogen, and sulfur, wherein the heterocyclyl is substituted with t occurrences of R⁴. In certain embodiments, R³ is a 5 membered monocyclic saturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 5 membered monocyclic saturated heterocyclyl containing 1 heteroatom selected from oxygen, nitrogen, and sulfur, wherein the heterocyclyl is substituted with t occurrences of R⁴. In certain embodiments, R³ is a 5 membered monocyclic saturated heterocyclyl containing 2 heteroatoms independently selected from oxygen, nitrogen, and sulfur, wherein the heterocyclyl is substituted with t occurrences of R⁴. In certain embodiments, R³ is a 5 membered monocyclic saturated heterocyclyl containing 3 heteroatoms independently selected from oxygen, nitrogen, and sulfur, wherein the heterocyclyl is substituted with t occurrences of R⁴.

In certain embodiments, R³ is a 6 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 6 membered monocyclic saturated heterocyclyl containing 1, 2, or 3 heteroatoms independently selected from oxygen, nitrogen, and sulfur, wherein the heterocyclyl is substituted with t occurrences of R. In certain embodiments, R³ is a 6 membered monocyclic saturated heterocyclyl containing 1 heteroatom selected from oxygen, nitrogen, and sulfur, wherein the heterocyclyl is substituted with t occurrences of R⁴. In certain embodiments, R³ is a 6 membered monocyclic saturated heterocyclyl containing 2 heteroatoms independently selected from oxygen, nitrogen, and sulfur, wherein the heterocyclyl is substituted with t occurrences of R⁴. In certain embodiments, R³ is a 6 membered monocyclic saturated heterocyclyl containing 3 heteroatoms independently selected from oxygen, nitrogen, and sulfur, wherein the heterocyclyl is substituted with t occurrences of R⁴. In certain embodiments, R³ is a 6 membered monocyclic partially unsaturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 6 membered monocyclic partially unsaturated heterocyclyl containing 1 heteroatom selected from oxygen, nitrogen, and sulfur, wherein the heterocyclyl is substituted with t occurrences of R⁴. In certain embodiments, R³ is a 6 membered monocyclic partially unsaturated heterocyclyl containing 2 heteroatoms independently selected from oxygen, nitrogen, and sulfur, wherein the heterocyclyl is substituted with t occurrences of R⁴. In certain embodiments, R³ is a 6 membered monocyclic partially unsaturated heterocyclyl containing 3 heteroatoms independently selected from oxygen, nitrogen, and sulfur, wherein the heterocyclyl is substituted with t occurrences of R⁴. In certain embodiments, R³ is a 6 membered bicyclic saturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 6 membered bicyclic saturated heterocyclyl containing 1 heteroatom selected from oxygen, nitrogen, and sulfur, wherein the heterocyclyl is substituted with t occurrences of R⁴. In certain embodiments, R³ is a 6 membered bicyclic saturated heterocyclyl containing 2 heteroatoms independently selected from oxygen, nitrogen, and sulfur, wherein the heterocyclyl is substituted with t occurrences of R⁴. In certain embodiments, R³ is a 6 membered bicyclic saturated heterocyclyl containing 3 heteroatoms independently selected from oxygen, nitrogen, and sulfur, wherein the heterocyclyl is substituted with t occurrences of R⁴. In certain embodiments, R³ is a 6 membered bicyclic partially unsaturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 6 membered bicyclic partially unsaturated heterocyclyl containing 1 heteroatom selected from oxygen, nitrogen, and sulfur, wherein the heterocyclyl is substituted with t occurrences of R⁴. In certain embodiments, R³ is a 6 membered bicyclic partially unsaturated heterocyclyl containing 2 heteroatoms independently selected from oxygen, nitrogen, and sulfur, wherein the heterocyclyl is substituted with t occurrences of R⁴. In certain embodiments, R³ is a 6 membered bicyclic partially unsaturated heterocyclyl containing 3 heteroatoms independently selected from oxygen, nitrogen, and sulfur, wherein the heterocyclyl is substituted with t occurrences of R⁴.

In certain embodiments, R³ is a 7 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 7 membered monocyclic saturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 7 membered monocyclic saturated heterocyclyl containing 1 heteroatom selected from oxygen, nitrogen, and sulfur, wherein the heterocyclyl is substituted with t occurrences of R⁴. In certain embodiments, R³ is a 7 membered monocyclic saturated heterocyclyl containing 2 heteroatoms independently selected from oxygen, nitrogen, and sulfur, wherein the heterocyclyl is substituted with t occurrences of R⁴. In certain embodiments, R³ is a 7 membered monocyclic saturated heterocyclyl containing 3 heteroatoms independently selected from oxygen, nitrogen, and sulfur, wherein the heterocyclyl is substituted with t occurrences of R⁴. In certain embodiments, R³ is a 7 membered monocyclic partially unsaturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 7 membered monocyclic partially unsaturated heterocyclyl containing 1 heteroatom selected from oxygen, nitrogen, and sulfur, wherein the heterocyclyl is substituted with t occurrences of R⁴. In certain embodiments, R³ is a 7 membered monocyclic partially unsaturated heterocyclyl containing 2 heteroatoms independently selected from oxygen, nitrogen, and sulfur, wherein the heterocyclyl is substituted with t occurrences of R⁴. In certain embodiments, R³ is a 7 membered monocyclic partially unsaturated heterocyclyl containing 3 heteroatoms independently selected from oxygen, nitrogen, and sulfur, wherein the heterocyclyl is substituted with t occurrences of R⁴. In certain embodiments, R³ is a 7 membered bicyclic saturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 7 membered bicyclic saturated heterocyclyl containing 1 heteroatom selected from oxygen, nitrogen, and sulfur, wherein the heterocyclyl is substituted with t occurrences of R⁴. In certain embodiments, R³ is a 7 membered bicyclic saturated heterocyclyl containing 2 heteroatoms independently selected from oxygen, nitrogen, and sulfur, wherein the heterocyclyl is substituted with t occurrences of R⁴. In certain embodiments, R³ is a 7 membered bicyclic saturated heterocyclyl containing 3 heteroatoms independently selected from oxygen, nitrogen, and sulfur, wherein the heterocyclyl is substituted with t occurrences of R⁴. In certain embodiments, R³ is a 7 membered bicyclic partially unsaturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 7 membered bicyclic partially unsaturated heterocyclyl containing 1 heteroatom selected from oxygen, nitrogen, and sulfur, wherein the heterocyclyl is substituted with t occurrences of R⁴. In certain embodiments, R³ is a 7 membered bicyclic partially unsaturated heterocyclyl containing 2 heteroatoms independently selected from oxygen, nitrogen, and sulfur, wherein the heterocyclyl is substituted with t occurrences of R⁴. In certain embodiments, R³ is a 7 membered bicyclic partially unsaturated heterocyclyl containing 3 heteroatoms independently selected from oxygen, nitrogen, and sulfur, wherein the heterocyclyl is substituted with t occurrences of R⁴.

In certain embodiments, R³ is a 8 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 8 membered monocyclic saturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 8 membered monocyclic saturated heterocyclyl containing 1 heteroatom selected from oxygen, nitrogen, and sulfur, wherein the heterocyclyl is substituted with t occurrences of R⁴. In certain embodiments, R³ is a 8 membered monocyclic saturated heterocyclyl containing 2 heteroatoms independently selected from oxygen, nitrogen, and sulfur, wherein the heterocyclyl is substituted with t occurrences of R⁴. In certain embodiments, R³ is a 8 membered monocyclic saturated heterocyclyl containing 3 heteroatoms independently selected from oxygen, nitrogen, and sulfur, wherein the heterocyclyl is substituted with t occurrences of R⁴. In certain embodiments, R³ is a 8 membered monocyclic partially unsaturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 8 membered monocyclic partially unsaturated heterocyclyl containing 1 heteroatom selected from oxygen, nitrogen, and sulfur, wherein the heterocyclyl is substituted with t occurrences of R⁴. In certain embodiments, R³ is a 8 membered monocyclic partially unsaturated heterocyclyl containing 2 heteroatoms independently selected from oxygen, nitrogen, and sulfur, wherein the heterocyclyl is substituted with t occurrences of R⁴. In certain embodiments, R³ is a 8 membered monocyclic partially unsaturated heterocyclyl containing 3 heteroatoms independently selected from oxygen, nitrogen, and sulfur, wherein the heterocyclyl is substituted with t occurrences of R⁴. In certain embodiments, R³ is a 8 membered bicyclic saturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 8 membered bicyclic saturated heterocyclyl containing 1 heteroatom selected from oxygen, nitrogen, and sulfur, wherein the heterocyclyl is substituted with t occurrences of R⁴. In certain embodiments, R³ is a 8 membered bicyclic saturated heterocyclyl containing 2 heteroatoms independently selected from oxygen, nitrogen, and sulfur, wherein the heterocyclyl is substituted with t occurrences of R⁴. In certain embodiments, R³ is a 8 membered bicyclic saturated heterocyclyl containing 3 heteroatoms independently selected from oxygen, nitrogen, and sulfur, wherein the heterocyclyl is substituted with t occurrences of R⁴. In certain embodiments, R³ is a 8 membered bicyclic partially unsaturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 8 membered bicyclic partially unsaturated heterocyclyl containing 1 heteroatom selected from oxygen, nitrogen, and sulfur, wherein the heterocyclyl is substituted with t occurrences of R⁴. In certain embodiments, R³ is a 8 membered bicyclic partially unsaturated heterocyclyl containing 2 heteroatoms independently selected from oxygen, nitrogen, and sulfur, wherein the heterocyclyl is substituted with t occurrences of R⁴. In certain embodiments, R³ is a 8 membered bicyclic partially unsaturated heterocyclyl containing 3 heteroatoms independently selected from oxygen, nitrogen, and sulfur, wherein the heterocyclyl is substituted with t occurrences of R⁴.

In certain embodiments, R³ is a 9 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 9 membered monocyclic saturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 9 membered monocyclic saturated heterocyclyl containing 1 heteroatom selected from oxygen, nitrogen, and sulfur, wherein the heterocyclyl is substituted with t occurrences of R⁴. In certain embodiments, R³ is a 9 membered monocyclic saturated heterocyclyl containing 2 heteroatoms independently selected from oxygen, nitrogen, and sulfur, wherein the heterocyclyl is substituted with t occurrences of R⁴. In certain embodiments, R³ is a 9 membered monocyclic saturated heterocyclyl containing 3 heteroatoms independently selected from oxygen, nitrogen, and sulfur, wherein the heterocyclyl is substituted with t occurrences of R⁴. In certain embodiments, R³ is a 9 membered monocyclic partially unsaturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 9 membered monocyclic partially unsaturated heterocyclyl containing 1 heteroatom selected from oxygen, nitrogen, and sulfur, wherein the heterocyclyl is substituted with t occurrences of R⁴. In certain embodiments, R³ is a 9 membered monocyclic partially unsaturated heterocyclyl containing 2 heteroatoms independently selected from oxygen, nitrogen, and sulfur, wherein the heterocyclyl is substituted with t occurrences of R⁴. In certain embodiments, R³ is a 9 membered monocyclic partially unsaturated heterocyclyl containing 3 heteroatoms independently selected from oxygen, nitrogen, and sulfur, wherein the heterocyclyl is substituted with t occurrences of R⁴. In certain embodiments, R³ is a 9 membered bicyclic saturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 9 membered bicyclic saturated heterocyclyl containing 1 heteroatom selected from oxygen, nitrogen, and sulfur, wherein the heterocyclyl is substituted with t occurrences of R⁴. In certain embodiments, R³ is a 9 membered bicyclic saturated heterocyclyl containing 2 heteroatoms independently selected from oxygen, nitrogen, and sulfur, wherein the heterocyclyl is substituted with t occurrences of R⁴. In certain embodiments, R³ is a 9 membered bicyclic saturated heterocyclyl containing 3 heteroatoms independently selected from oxygen, nitrogen, and sulfur, wherein the heterocyclyl is substituted with t occurrences of R⁴. In certain embodiments, R³ is a 9 membered bicyclic partially unsaturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 9 membered bicyclic partially unsaturated heterocyclyl containing 1 heteroatom selected from oxygen, nitrogen, and sulfur, wherein the heterocyclyl is substituted with t occurrences of R⁴. In certain embodiments, R³ is a 9 membered bicyclic partially unsaturated heterocyclyl containing 2 heteroatoms independently selected from oxygen, nitrogen, and sulfur, wherein the heterocyclyl is substituted with t occurrences of R⁴. In certain embodiments, R³ is a 9 membered bicyclic partially unsaturated heterocyclyl containing 3 heteroatoms independently selected from oxygen, nitrogen, and sulfur, wherein the heterocyclyl is substituted with t occurrences of R⁴.

In certain embodiments, R³ is a 10 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 10 membered monocyclic saturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 10 membered monocyclic saturated heterocyclyl containing 1 heteroatom selected from oxygen, nitrogen, and sulfur, wherein the heterocyclyl is substituted with t occurrences of R⁴. In certain embodiments, R³ is a 10 membered monocyclic saturated heterocyclyl containing 2 heteroatoms independently selected from oxygen, nitrogen, and sulfur, wherein the heterocyclyl is substituted with t occurrences of R⁴. In certain embodiments, R³ is a 10 membered monocyclic saturated heterocyclyl containing 3 heteroatoms independently selected from oxygen, nitrogen, and sulfur, wherein the heterocyclyl is substituted with t occurrences of R⁴. In certain embodiments, R³ is a 10 membered monocyclic partially unsaturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 10 membered monocyclic partially unsaturated heterocyclyl containing 1 heteroatom selected from oxygen, nitrogen, and sulfur, wherein the heterocyclyl is substituted with t occurrences of R⁴. In certain embodiments, R³ is a 10 membered monocyclic partially unsaturated heterocyclyl containing 2 heteroatoms independently selected from oxygen, nitrogen, and sulfur, wherein the heterocyclyl is substituted with t occurrences of R⁴. In certain embodiments, R³ is a 10 membered monocyclic partially unsaturated heterocyclyl containing 3 heteroatoms independently selected from oxygen, nitrogen, and sulfur, wherein the heterocyclyl is substituted with t occurrences of R⁴. In certain embodiments, R³ is a 10 membered bicyclic saturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 10 membered bicyclic saturated heterocyclyl containing 1 heteroatom selected from oxygen, nitrogen, and sulfur, wherein the heterocyclyl is substituted with t occurrences of R⁴. In certain embodiments, R³ is a 10 membered bicyclic saturated heterocyclyl containing 2 heteroatoms independently selected from oxygen, nitrogen, and sulfur, wherein the heterocyclyl is substituted with t occurrences of R⁴. In certain embodiments, R³ is a 10 membered bicyclic saturated heterocyclyl containing 3 heteroatoms independently selected from oxygen, nitrogen, and sulfur, wherein the heterocyclyl is substituted with t occurrences of R⁴. In certain embodiments, R³ is a 10 membered bicyclic partially unsaturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 10 membered bicyclic partially unsaturated heterocyclyl containing 1 heteroatom selected from oxygen, nitrogen, and sulfur, wherein the heterocyclyl is substituted with t occurrences of R⁴. In certain embodiments, R³ is a 10 membered bicyclic partially unsaturated heterocyclyl containing 2 heteroatoms independently selected from oxygen, nitrogen, and sulfur, wherein the heterocyclyl is substituted with t occurrences of R⁴. In certain embodiments, R³ is a 10 membered bicyclic partially unsaturated heterocyclyl containing 3 heteroatoms independently selected from oxygen, nitrogen, and sulfur, wherein the heterocyclyl is substituted with t occurrences of R⁴.

In certain embodiments, R³ is C₂₋₆ alkyl. In certain embodiments, R³ is hydroxyl. In certain embodiments, R³ is tetrahydropyranyl substituted with t occurrences of R⁴. In certain embodiments, R³ is morpholinyl substituted with t occurrences of R⁴. In certain embodiments, R³ is piperidinyl substituted with t occurrences of R⁴. In certain embodiments, R³ is piperazinyl substituted with t occurrences of R⁴. In certain embodiments, R³ is selected from the groups depicted in the compounds in Table 1 below.

As defined generally above, R⁴ represents independently for each occurrence halo, hydroxyl, C₁₋₆ alkyl, C₁₋₆ haloalkyl, C₁₋₆ hydroxyalkyl, C₁₋₆ alkoxyl, C₃₋₇ cycloalkyl, —C(O)R¹, —C(O)N(R⁹)(R¹⁰), cyano, —N(R¹²)(R¹³), or —N(R⁹)C(O)R¹¹. In certain embodiments, R⁴ represents independently for each occurrence halo, hydroxyl, C₁₋₆ alkyl, C₁₋₆ haloalkyl, or C₃₋₇ cycloalkyl. In certain embodiments, R⁴ represents independently for each occurrence halo or C₁₋₆ alkyl. In certain embodiments, R⁴ is halo. In certain embodiments, R⁴ is hydroxyl. In certain embodiments, R⁴ is C₁₋₆ alkyl. In certain embodiments, R⁴ is C₁₋₆ haloalkyl. In certain embodiments, R⁴ is C₁₋₆ hydroxyalkyl. In certain embodiments, R⁴ is C₁₋₆ alkoxyl. In certain embodiments, R⁴ is C₃₋₇ cycloalkyl. In certain embodiments, R⁴ is —C(O)R¹¹. In certain embodiments, R⁴ is —C(O)N(R⁹)(R¹⁰). In certain embodiments, R⁴ is cyano. In certain embodiments, R⁴ is —N(R¹²)(R¹³). In certain embodiments, R⁴ is —N(R⁹)C(O)R¹¹. In certain embodiments, R⁴ represents independently for each occurrence halo, hydroxyl, C₁. alkyl, C₁₋₆ haloalkyl, C₁₋₆ hydroxyalkyl, C₁₋₆ alkoxyl, C₃₋₇ cycloalkyl, —C(O)R¹¹, —C(O)N(R⁹)(R¹⁰), or —N(R⁹)C(O)R¹¹. In certain embodiments, R⁴ is selected from the groups depicted in the compounds in Table 1 below.

As defined generally above, R^{•} is a 5-6 membered heteroaryl containing 1, 2, or 3 heteroatoms independently selected from oxygen, nitrogen, and sulfur, or a 3-7 membered saturated heterocyclyl containing 1, 2, or 3 heteroatoms independently selected from oxygen, nitrogen, and sulfur, wherein the heteroaryl and heterocyclyl are substituted with q occurrences of R^{T}. In certain embodiments, R⁵ is a 5 membered heteroaryl containing 1, 2, or 3 heteroatoms independently selected from oxygen, nitrogen, and sulfur, wherein the heteroaryl is substituted with q occurrences of R⁷. In certain embodiments, R⁵ is a 1,2,3-triazolyl, pyrazolyl, oxazolyl, imidazolyl, isoxazolyl, pyrrolyl, or furanyl, each of which substituted with q occurrences of R⁷. In certain embodiments, R⁵ is 1,2,3-triazolyl substituted with q occurrences of R⁷. In certain embodiments, R⁵ is a 5 membered heteroaryl containing 1 heteroatom selected from oxygen, nitrogen, and sulfur, wherein the heteroaryl is substituted with q occurrences of R⁷. In certain embodiments, R⁵ is a 5 membered heteroaryl containing 2 heteroatoms independently selected from oxygen, nitrogen, and sulfur, wherein the heteroaryl is substituted with q occurrences of R⁷. In certain embodiments, R⁵ is a 5 membered heteroaryl containing 3 heteroatoms independently selected from oxygen, nitrogen, and sulfur, wherein the heteroaryl is substituted with q occurrences of R⁷. In certain embodiments, R⁵ is a 6 membered heteroaryl containing 1, 2, or 3 heteroatoms independently selected from oxygen, nitrogen, and sulfur, wherein the heteroaryl is substituted with q occurrences of R⁷. In certain embodiments, R⁵ is a 6 membered heteroaryl containing 1 heteroatom selected from oxygen, nitrogen, and sulfur, wherein the heteroaryl is substituted with q occurrences of R⁷. In certain embodiments, R⁵ is a 6 membered heteroaryl containing 2 heteroatoms independently selected from oxygen, nitrogen, and sulfur, wherein the heteroaryl is substituted with q occurrences of R⁷. In certain embodiments, R⁵ is a 6 membered heteroaryl containing 3 heteroatoms independently selected from oxygen, nitrogen, and sulfur, wherein the heteroaryl is substituted with q occurrences of R⁷. In certain embodiments, R⁵ is a pyrazolyl substituted with q occurrences of R⁷. In certain embodiments, R⁵ is an oxazolyl substituted with q occurrences of R⁷. In certain embodiments, R⁵ is an imidazolyl substituted with q occurrences of R⁷. In certain embodiments, R⁵ is an isoxazolyl substituted with q occurrences of R⁷. In certain embodiments, R^{○} is a pyrrolyl substituted with q occurrences of R⁷. In certain embodiments, R⁵ is a furanyl substituted with q occurrences of R⁷. In certain embodiments, R⁵ is selected from the groups depicted in the compounds in Table 1 below.

As defined generally above, R⁶ represents independently for each occurrence halo, hydroxyl, C₁₋₆ alkyl. C₁₋₆ haloalkyl, C₁₋₆ hydroxyalkyl, C₁₋₆ alkoxyl, C₃₋₇ cycloalkyl, cyano, —O—C₃₋₇ cycloalkyl, —N(R⁹)(R¹⁰), —(C₀₋₄ alkylene)-C(O)R⁸, —C(O)N(R⁹)(R¹⁰), or —N(R⁹)C(O)R¹¹. In certain embodiments, R⁶ represents independently for each occurrence halo, C₁₋₆ alkyl, C₁₋₆ haloalkyl, cyano, or —(C₀₋₄ alkylene)-C(O)R¹¹. In certain embodiments, R⁶ represents independently for each occurrence halo, cyano, or —(C₀₋₄ alkylene)-C(O)R⁸. In certain embodiments, R⁶ is chloro. In certain embodiments, R⁶ is C₁₋₆ haloalkyl. In certain embodiments, R⁶ is —CF₃. In certain embodiments, R⁶ is halo. In certain embodiments, R⁶ is Br. In certain embodiments, R⁶ is F. In certain embodiments, R⁶ is I. In certain embodiments, R⁶ is hydroxyl. In certain embodiments, R⁶ is C₁₋₆ alkyl. In certain embodiments, R⁶ is C₁₋₆ hydroxyalkyl. In certain embodiments, R⁶ is C₁₋₆ alkoxyl. In certain embodiments, R⁶ is C₃₋₇ cycloalkyl. In certain embodiments, R⁶ is cyano. In certain embodiments, R⁶ is —O—C₃₋₇ cycloalkyl. In certain embodiments, R⁶ is —N(R⁹)(R¹⁰). In certain embodiments, R⁶ is —(C₀₋₄ alkylene)-C(O)R⁸. In certain embodiments, R⁶ is —C(O)N(R⁹)(R¹⁰). In certain embodiments, R⁶ is —N(R⁹)C(O)R¹¹. In certain embodiments, R⁶ is selected from the groups depicted in the compounds in Table 1 below.

As defined generally above, R⁷ represents independently for each occurrence halo, hydroxyl, C₁₋₆ alkyl, C₁₋₆ haloalkyl, C₁₋₆ alkoxyl, or C₃₋₇ cycloalkyl. In certain embodiments, R⁷ is halo. In certain embodiments, R⁷ is hydroxyl. In certain embodiments, R⁷ is C₁₋₆ alkyl. In certain embodiments, R⁷ is C₁₋₆ haloalkyl. In certain embodiments, R⁷ is C₁₋₆ alkoxyl. In certain embodiments, R⁷ is C₃₋₇ cycloalkyl. In certain embodiments, R⁷ is selected from the groups depicted in the compounds in Table 1 below.

As defined generally above, R⁸ is —OH, —O—(C₁₋₆ alkyl), —O—C₃₋₇ cycloalkyl, or A². In certain embodiments, R⁸ is —OH. In certain embodiments, R⁸ is —O—(C₁₋₆ alkyl). In certain embodiments, R⁸ is —O—C₃₋₇ cycloalkyl. In certain embodiments, R⁸ is A². In certain embodiments, R⁸ is selected from the groups depicted in the compounds in Table 1 below.

As defined generally above, R⁹ and R¹⁰ are independently hydrogen, C₁₋₆ alkyl, C₃₋₇ cycloalkyl, C₂₋₄ hydroxyalkyl, or —(C₂₋₆ alkylene)-(C₁₋₆ alkoxyl), or R⁹ and R¹⁰ are taken together with the nitrogen atom to which they are attached to form a 3-7 membered heterocyclic ring containing 1 nitrogen atom, wherein the heterocyclic ring is substituted with 0 or 1 groups independently selected from hydroxyl, halo, and C₁₋₆ alkyl. In certain embodiments, R⁹ is hydrogen. In certain embodiments, R⁹ is C₁₋₆ alkyl. In certain embodiments, R⁹ is C₃₋₇ cycloalkyl. In certain embodiments, R⁹ is C₂₋₄hydroxyalkyl. In certain embodiments, R⁹ is —(C₂₋₆ alkylene)-(C₁₋₆ alkoxyl). In certain embodiments, R¹⁰ is hydrogen. In certain embodiments, R¹⁰ is C₁₋₆ alkyl. In certain embodiments, R¹⁰ is C₃₋₇ cycloalkyl. In certain embodiments, R¹⁰ is C₂₋₄ hydroxyalkyl. In certain embodiments, R¹⁰ is —(C₂₋₆ alkylene)-(C₁₋₆ alkoxyl). In certain embodiments, R⁹ and R¹⁰ are taken together with the nitrogen atom to which they are attached to form a 3-7 membered heterocyclic ring containing 1 nitrogen atom, wherein the heterocyclic ring is substituted with 0 or 1 groups independently selected from hydroxyl, halo, and C₁₋₆ alkyl. In certain embodiments, R⁹ and R¹⁰ are taken together with the nitrogen atom to which they are attached to form azetidinyl substituted by hydroxyl. In certain embodiments, R⁹ is selected from the groups depicted in the compounds in Table 1 below. In certain embodiments, R¹⁰ is selected from the groups depicted in the compounds in Table 1 below.

As defined generally above, R¹¹ represents independently for each occurrence C₁₋₆ alkyl or (Co-s alkylene)-C₃₋₇ cycloalkyl. In certain embodiments, R¹¹ is C₁₋₆ alkyl. In certain embodiments, R¹¹ is C₁ alkyl. In certain embodiments, R¹¹ is C₂ alkyl. In certain embodiments, R¹¹ is C₃ alkyl. In certain embodiments, R¹¹ is C₄ alkyl. In certain embodiments, R¹¹ is C₅ alkyl. In certain embodiments, R¹¹ is C₆ alkyl. In certain embodiments, R¹¹ is (C₀₋₅ alkylene)-C₃₋₇ cycloalkyl. In certain embodiments, R¹¹ is selected from the groups depicted in the compounds in Table 1 below.

As defined generally above, R¹² and R¹³ are independently hydrogen, C₁₋₆ alkyl, or C₃₋₅ cycloalkyl, or R¹² and R¹³ are taken together with the nitrogen atom to which they are attached to form a 3-7 membered heterocyclic ring containing 1 nitrogen atom. In certain embodiments, R¹² is hydrogen. In certain embodiments, R¹² is C₁₋₆ alkyl. In certain embodiments, R¹² is C₁ alkyl. In certain embodiments, R¹² is C₂ alkyl. In certain embodiments, R¹² is C₃ alkyl. In certain embodiments, R¹² is C₄ alkyl. In certain embodiments, R¹² is C₅ alkyl. In certain embodiments, R¹² is C₆ alkyl. In certain embodiments, R¹² is C₃₋₅ cycloalkyl. In certain embodiments, R¹² is C₃ cycloalkyl. In certain embodiments, R¹² is C₄ cycloalkyl. In certain embodiments, R¹² is C₅ cycloalkyl. In certain embodiments, R¹³ is hydrogen. In certain embodiments, R¹³ is C₁₋₆ alkyl. In certain embodiments, R¹³ is C₁ alkyl. In certain embodiments, R¹³ is C₂ alkyl. In certain embodiments, R¹³ is C₃ alkyl. In certain embodiments, R¹³ is C₄ alkyl. In certain embodiments, R¹³ is C₅ alkyl. In certain embodiments, R¹³ is C₆ alkyl. In certain embodiments, R¹³ is C₃₋₅ cycloalkyl. In certain embodiments, R¹³ is C₃ cycloalkyl. In certain embodiments, R¹³ is C₄ cycloalkyl. In certain embodiments, R¹³ is C₅ cycloalkyl. In certain embodiments, R¹² and R¹³ are taken together with the nitrogen atom to which they are attached to form a 3-7 membered heterocyclic ring containing 1 nitrogen atom. In certain embodiments. R¹² and R¹³ are taken together with the nitrogen atom to which they are attached to form a 3 membered heterocyclic ring containing 1 nitrogen atom. In certain embodiments, R¹² and R¹³ are taken together with the nitrogen atom to which they are attached to form a 4 membered heterocyclic ring containing 1 nitrogen atom. In certain embodiments, R¹² and R¹³ are taken together with the nitrogen atom to which they are attached to form a 5 membered heterocyclic ring containing 1 nitrogen atom. In certain embodiments, R¹² and R¹³ are taken together with the nitrogen atom to which they are attached to form a 6 membered heterocyclic ring containing 1 nitrogen atom. In certain embodiments, R¹² and R¹³ are taken together with the nitrogen atom to which they are attached to form a 7 membered heterocyclic ring containing 1 nitrogen atom. In certain embodiments, R¹² is selected from the groups depicted in the compounds in Table 1 below. In certain embodiments, R¹³ is selected from the groups depicted in the compounds in Table 1 below.

As defined generally above, m is 0 or 1. In certain embodiments, m is 1. In certain embodiments, m is 0. In certain embodiments, m is selected from the corresponding value in the groups depicted in the compounds in Table 1 below.

As defined generally above, n, q, and t are independently 0, 1, or 2. In certain embodiments, n is 1. In certain embodiments, n is 0. In certain embodiments, q is 0. In certain embodiments, t is 1. In certain embodiments, t is 0. In certain embodiments, n is 2. In certain embodiments, q is 1. In certain embodiments, q is 2. In certain embodiments, t is 2. In certain embodiments, y is 0. In certain embodiments, y is 1. In certain embodiments, y is 2. In certain embodiments, n is selected from the corresponding value in the groups depicted in the compounds in Table 1 below. In certain embodiments, q is selected from the corresponding value in the groups depicted in the compounds in Table 1 below. In certain embodiments, t is selected from the corresponding value in the groups depicted in the compounds in Table 1 below.

The description above describes multiple embodiments relating to compounds of Formula I. The patent application specifically contemplates all combinations of the embodiments.

In certain embodiments, the compound of Formula I is further defined by Formula Ia or a pharmaceutically acceptable salt thereof:

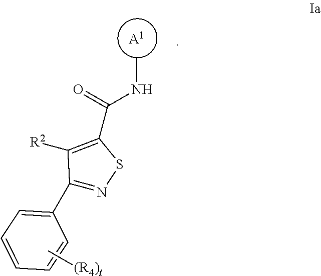

In certain embodiments, the definition of variables R², R⁴, A¹, and t is one of the embodiments described above in connection with Formula I.

The description above describes multiple embodiments relating to compounds of Formula Ia. The patent application specifically contemplates all combinations of the embodiments.

In certain embodiments, the compound of Formula I is further defined by Formula Ib or Ic or a pharmaceutically acceptable salt thereof:

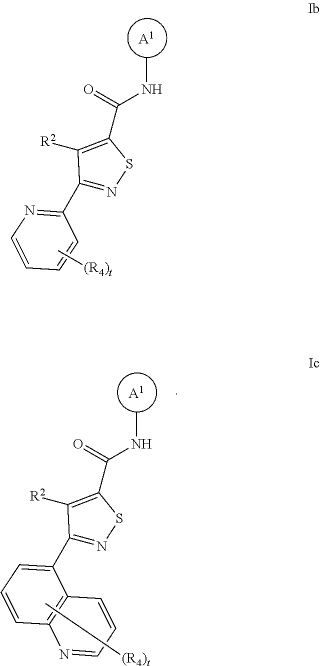

In certain embodiments, the definition of variables R², R⁴, A¹, and t is one of the embodiments described above in connection with Formula I. In certain embodiments, the compound of Formula I is further defined by Formula Ib or a pharmaceutically acceptable salt thereof. In certain embodiments, the compound of Formula I is further defined by Formula Ic or a pharmaceutically acceptable salt thereof.

The description above describes multiple embodiments relating to compounds of Formulae Tb and Ic. The patent application specifically contemplates all combinations of the embodiments.

In certain embodiments, the compound of Formula I is further defined by Formula Id or a pharmaceutically acceptable salt thereof:

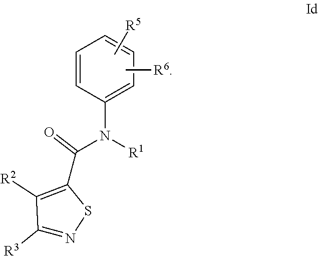

In certain embodiments, the definition of variables R¹, R², R³, R⁵, and R⁶ is one of the embodiments described above in connection with Formula I.

The description above describes multiple embodiments relating to compounds of Formula Id. The patent application specifically contemplates all combinations of the embodiments.

In certain embodiments, the compound of Formula I is further defined by Formula Ie or a pharmaceutically acceptable salt thereof:

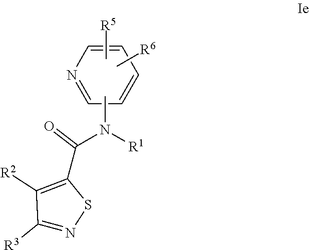

In certain embodiments, the definition of variables R¹, R², R³, R⁵, and R⁶ is one of the embodiments described above in connection with Formula I.

The description above describes multiple embodiments relating to compounds of Formula Ie. The patent application specifically contemplates all combinations of the embodiments.

In certain embodiments, the compound of Formula I is further defined by Formula If or a pharmaceutically acceptable salt thereof:

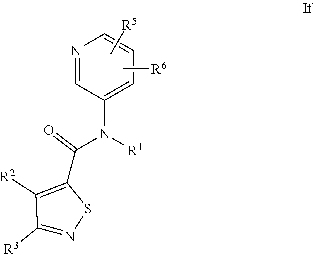

In certain embodiments, the definition of variables R¹, R², R³, R⁵, and R⁶ is one of the embodiments described above in connection with Formula I.

The description above describes multiple embodiments relating to compounds of Formula If. The patent application specifically contemplates all combinations of the embodiments.

In certain embodiments, the compound of Formula I is further defined by Formula Ig or Ih or a pharmaceutically acceptable salt thereof:

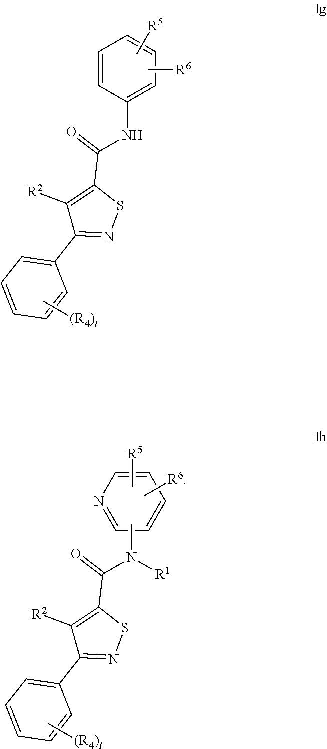

In certain embodiments, the definition of variables R², R⁴, R⁵, R⁶, and t is one of the embodiments described above in connection with Formula I. In certain embodiments, the compound of Formula I is further defined by Formula Ig or a pharmaceutically acceptable salt thereof. In certain embodiments, the compound of Formula I is further defined by Formula Ih or a pharmaceutically acceptable salt thereof.

The description above describes multiple embodiments relating to compounds of Formulae Ig and Ih. The patent application specifically contemplates all combinations of the embodiments.

In certain embodiments, the compound of Formula I is further defined by Formula Ii, Ij, Ik, or Il or a pharmaceutically acceptable salt thereof.

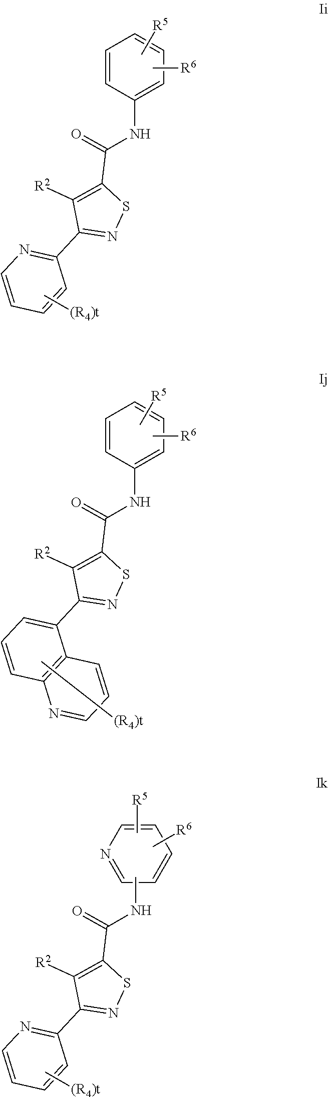

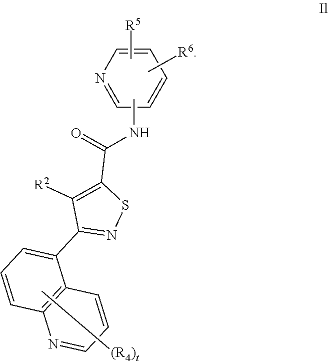

In certain embodiments, the definition of variables R², R⁴, R⁵, R⁶, and t is one of the embodiments described above in connection with Formula I. In certain embodiments, the compound of Formula I is further defined by Formula Ii or a pharmaceutically acceptable salt thereof. In certain embodiments, the compound of Formula I is further defined by Formula Ij or a pharmaceutically acceptable salt thereof. In certain embodiments, the compound of Formula I is further defined by Formula Ik or a pharmaceutically acceptable salt thereof. In certain embodiments, the compound of Formula I is further defined by Formula Ii or a pharmaceutically acceptable salt thereof.

The description above describes multiple embodiments relating to compounds of Formulae Ii, Ij, Ik, and Ii. The patent application specifically contemplates all combinations of the embodiments.

In certain embodiments, the compound of Formula I is further defined by Formula Im or In or a pharmaceutically acceptable salt thereof.

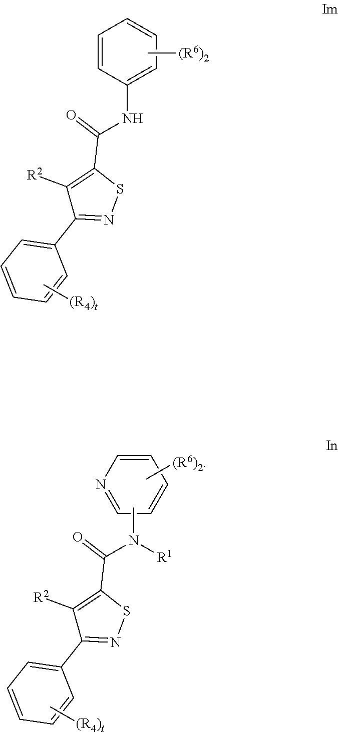

In certain embodiments, the definition of variables R², R⁴, R⁶, and t is one of the embodiments described above in connection with Formula I. In certain embodiments, the compound of Formula I is further defined by Formula Im or a pharmaceutically acceptable salt thereof. In certain embodiments, the compound of Formula I is further defined by Formula In or a pharmaceutically acceptable salt thereof.

The description above describes multiple embodiments relating to compounds of Formulae Im and In. The patent application specifically contemplates all combinations of the embodiments.

In certain embodiments, the compound of Formula I is further defined by Formula Io, Ip, Iq, or Ir or a pharmaceutically acceptable salt thereof:

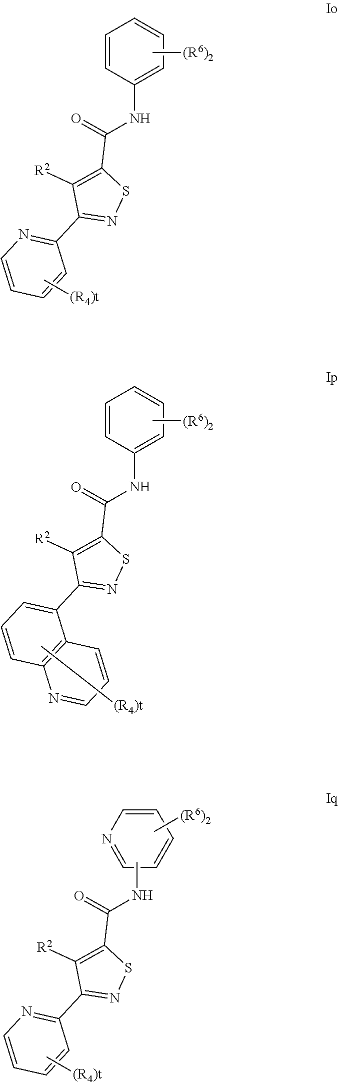

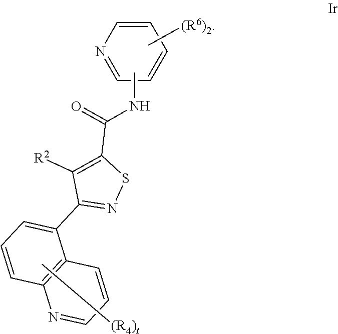

In certain embodiments, the definition of variables R², R⁴, R⁶, and t is one of the embodiments described above in connection with Formula I. In certain embodiments, the compound of Formula I is further defined by Formula Io or a pharmaceutically acceptable salt thereof. In certain embodiments, the compound of Formula I is further defined by Formula Ip or a pharmaceutically acceptable salt thereof. In certain embodiments, the compound of Formula I is further defined by Formula Iq or a pharmaceutically acceptable salt thereof. In certain embodiments, the compound of Formula I is further defined by Formula Ir or a pharmaceutically acceptable salt thereof.

The description above describes multiple embodiments relating to compounds of Formulae To, Ip, Iq, and Ir. The patent application specifically contemplates all combinations of the embodiments.

In certain embodiments, the compound of Formula I is further defined by Formula Is or It or a pharmaceutically acceptable salt thereof:

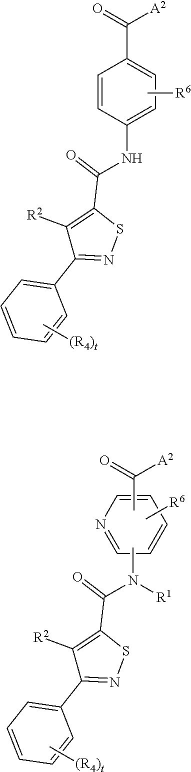

In certain embodiments, the definition of variables R¹, R², R⁴, R⁶, A², and t is one of the embodiments described above in connection with Formula I. In certain embodiments, the compound of Formula I is further defined by Formula Is or a pharmaceutically acceptable salt thereof. In certain embodiments, the compound of Formula I is further defined by Formula It or a pharmaceutically acceptable salt thereof.

The description above describes multiple embodiments relating to compounds of Formulae Is and It. The patent application specifically contemplates all combinations of the embodiments.

In certain embodiments, the compound of Formula I is further defined by Formula Iu, Iv, Iw, or Ix or a pharmaceutically acceptable salt thereof.

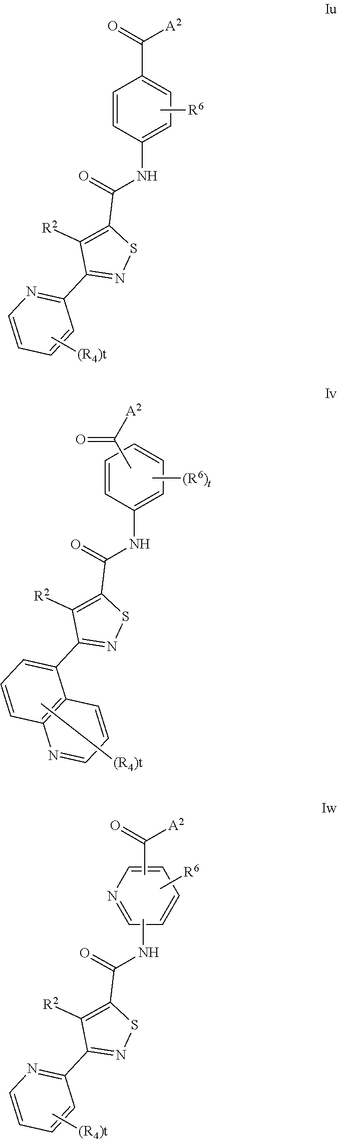

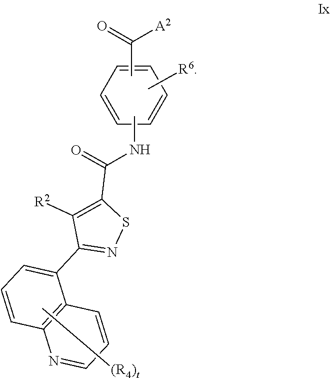

In certain embodiments, the definition of variables R², R⁴, R⁶, A², and t is one of the embodiments described above in connection with Formula I. In certain embodiments, the compound of Formula I is further defined by Formula Iu or a pharmaceutically acceptable salt thereof. In certain embodiments, the compound of Formula I is further defined by Formula Iv or a pharmaceutically acceptable salt thereof. In certain embodiments, the compound of Formula I is further defined by Formula Iw or a pharmaceutically acceptable salt thereof. In certain embodiments, the compound of Formula I is further defined by Formula Ix or a pharmaceutically acceptable salt thereof.

The description above describes multiple embodiments relating to compounds of Formulae Iu, Iv, Iw, and Ix. The patent application specifically contemplates all combinations of the embodiments.

In certain embodiments, the compound of Formula I is further defined by Formula Iy or a pharmaceutically acceptable salt thereof:

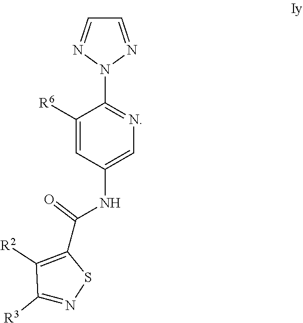

In certain embodiments, the definition of variables R², R³, and R⁶ is one of the embodiments described above in connection with Formula I. In certain embodiments, R³ is phenyl or

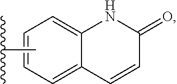

R² is C₁₋₄ haloalkyl or C₃₋₆ cycloalkyl, and R⁶ is halo or cyano. In certain embodiments, the compound of Formula I is further defined by Formula Iy.

The description above describes multiple embodiments relating to compounds of Formula Iy. The patent application specifically contemplates all combinations of the embodiments.

In certain embodiments, the compound of Formula I is further defined by Formula Iz or a pharmaceutically acceptable salt thereof:

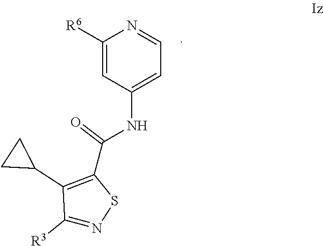

In certain embodiments, the definition of variables R³ and R⁶ is one of the embodiments described above in connection with Formula I. In certain embodiments, R³ is or

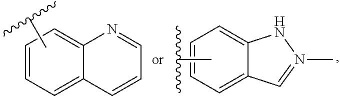

and R⁶ is C₁₋₄ alkyl or C₁₋₄ haloalkyl. In certain embodiments, the compound of Formula I is further defined by Formula Iz.

The description above describes multiple embodiments relating to compounds of Formula Iz. The patent application specifically contemplates all combinations of the embodiments.

Another aspect of the invention provides a compound represented by Formula I-1:

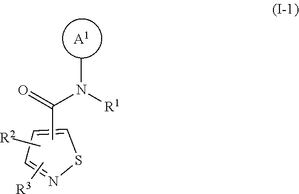

or a pharmaceutically acceptable salt thereof, wherein:
A¹ is phenyl, a 6 membered heteroaryl containing 1 or 2 nitrogen atoms, a 9-10 membered bicyclic heteroaryl containing 1, 2, or 3 heteroatoms independently selected from nitrogen and oxygen,

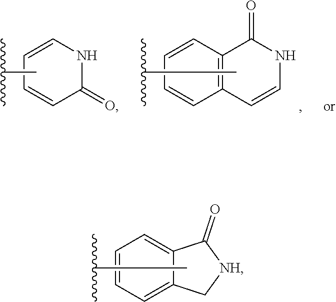

wherein the phenyl, heteroaryl,

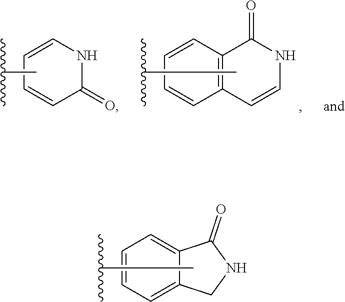

are substituted with m occurrences of R⁵ and n occurrences of R⁶;
A² represents independently for each occurrence a 4-6 membered saturated heterocyclyl containing 1 or 2 heteroatoms independently selected from oxygen, nitrogen, and sulfur, wherein the heterocyclyl is substituted with y occurrences of R⁷;
R¹ is hydrogen or C₁₋₄ alkyl;
R² is C₁₋₆ haloalkyl, C₁₋₆ alkyl, C₃₋₇ cycloalkyl, C₁₋₆ alkoxyl, cyano, C₂₋₄ alkenyl, halogen, hydrogen, oxetanyl, tetrahydrofuranyl, or —N(R¹²)(R¹³), wherein the oxetanyl and tetrahydrofuranyl are substituted with 0 or 1 hydroxyl groups;
R³ is one of the following:
(a) phenyl, a 5-6 membered heteroaryl containing 1, 2, or 3 heteroatoms independently selected from oxygen, nitrogen, and sulfur, a 9-10 membered bicyclic heteroaryl containing 1, 2, or 3 heteroatoms independently selected from oxygen, nitrogen, and sulfur, a 3-10 membered monocyclic or bicyclic saturated or partially unsaturated heterocyclyl containing 1, 2, or 3 heteroatoms independently selected from oxygen, nitrogen, and sulfur,

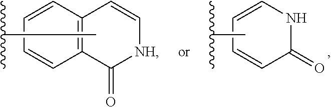

wherein the phenyl, heteroaryl, heterocyclyl,

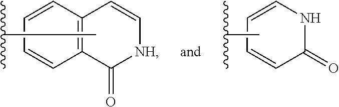

are substituted with t occurrences of R⁴; or
(b) C₂₋₆ alkyl, hydroxyl, or —N(R⁹)(R¹⁰) R⁴ represents independently for each occurrence halo, hydroxyl, C₁₋₆ alkyl, C₁₋₆ haloalkyl, C₁₋₆hydroxyalkyl, C₁₋₆ alkoxyl, C₃₋₇ cycloalkyl, —C(O)R¹¹, —C(O)N(R⁹)(R¹⁰), or —N(R⁹)C(O)R¹¹;
R⁵ is a 5-6 membered heteroaryl containing 1, 2, or 3 heteroatoms independently selected from oxygen, nitrogen, and sulfur, or a 3-7 membered saturated heterocyclyl containing 1, 2, or 3 heteroatoms independently selected from oxygen, nitrogen, and sulfur, wherein the heteroaryl and heterocyclyl are substituted with q occurrences of R⁷;
R⁶ represents independently for each occurrence halo, hydroxyl, C₁₋₆ alkyl, C₁₋₆ haloalkyl, C₁₋₆hydroxyalkyl, C₁₋₆ alkoxyl, C₃₋₇ cycloalkyl, cyano, —O—C₃₋₇ cycloalkyl, —N(R⁹)(R¹⁰), —(C₀₋₄ alkylene)-C(O)R⁸, —C(O)N(R⁹)(R¹¹), or —N(R⁹)C(O)R¹¹;
R⁷ represents independently for each occurrence halo, hydroxyl, C₁₋₆ alkyl, C₁₋₆ haloalkyl, C₁₋₆ alkoxyl, or C₃₋₇ cycloalkyl;
R⁸ is —OH, —O—(C₁₋₆ alkyl), —O—C₃₋₇ cycloalkyl, or A²;
R⁹ and R¹⁰ are independently hydrogen, C₁₋₆ alkyl, C₃₋₇ cycloalkyl, C₂₋₄ hydroxyalkyl, or —(C₂₋₆ alkylene)-(C₁₋₆ alkoxyl), or R⁹ and R¹⁰ are taken together with the nitrogen atom to which they are attached to form a 3-7 membered heterocyclic ring containing 1 nitrogen atom, wherein the heterocyclic ring is substituted with 0 or 1 groups independently selected from hydroxyl, halo, and C₁₋₆ alkyl;
R¹¹ represents independently for each occurrence C₁₋₆ alkyl or (C₀₋₅ alkylene)-C₃₋₇ cycloalkyl;
R¹² and R¹³ are independently hydrogen, C₁₋₆ alkyl, or C₃₋₅ cycloalkyl, or R¹² and R¹³ are taken together with the nitrogen atom to which they are attached to form a 3-7 membered heterocyclic ring containing 1 nitrogen atom;
m is 0 or 1; and
n, q, t, and y are independently 0, 1, or 2;
provided that when R² is hydrogen and m is 1, then R⁵ is 1,2,3-triazolyl substituted with q occurrences of R⁷.

The definitions of variables in Formula I-1 above encompass multiple chemical groups. The application contemplates embodiments where, for example, (i) the definition of a variable is a single chemical group selected from those chemical groups set forth above, (ii) the definition of a variable is a collection of two or more of the chemical groups selected from those set forth above, and (iii) the compound is defined by a combination of variables in which the variables are defined by (i) or (ii).

In certain embodiments, the compound is a compound of Formula I-1.

As defined generally above, A¹ is phenyl, a 6 membered heteroaryl containing 1 or 2 nitrogen atoms, a 9-10 membered bicyclic heteroaryl containing 1, 2, or 3 heteroatoms independently selected from nitrogen and oxygen,

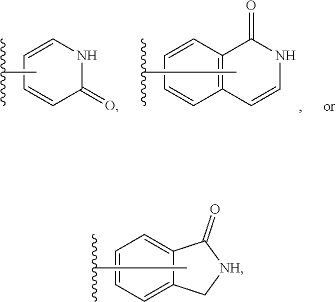

wherein the phenyl, heteroaryl,

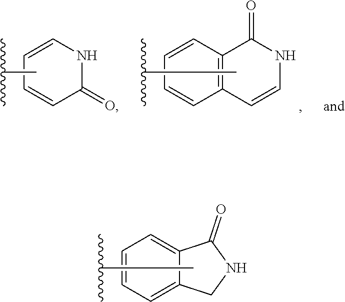

are substituted with m occurrences of R⁵ and n occurrences of R⁶. In certain embodiments, A¹ is phenyl substituted with m occurrences of R⁵ and n occurrences of R⁶. In certain embodiments, A¹ is a 6 membered heteroaryl containing 1 or 2 nitrogen atoms, wherein the heteroaryl is substituted with m occurrences of R⁵ and n occurrences of R⁶. In certain embodiments, A¹ is a pyridinyl substituted with m occurrences of R^{•} and n occurrences of R⁶. In certain embodiments, A¹ is a 9-10 membered bicyclic heteroaryl containing 1, 2, or 3 heteroatoms independently selected from nitrogen and oxygen, wherein the heteroaryl is substituted with m occurrences of R⁵ and n occurrences of R⁶. In certain embodiments, A¹ is

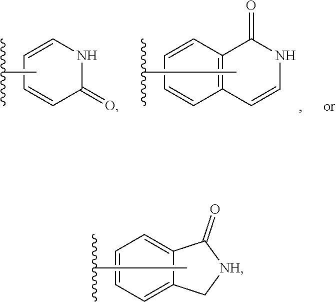

each of which is substituted with m occurrences of R⁵ and n occurrences of R⁶. In certain embodiments, A¹ is a 6 membered heteroaryl containing 1 nitrogen atom, wherein the heteroaryl is substituted with m occurrences of R⁵ and n occurrences of R⁶. In certain embodiments, A¹ is a 6 membered heteroaryl containing 2 nitrogen atoms, wherein the heteroaryl is substituted with m occurrences of R⁵ and n occurrences of R⁶. In certain embodiments, A¹ is 9 membered bicyclic heteroaryl containing 1, 2, or 3 heteroatoms independently selected from nitrogen and oxygen, wherein the heteroaryl is substituted with m occurrences of R⁵ and n occurrences of R⁶. In certain embodiments, A¹ is 9 membered bicyclic heteroaryl containing 1 heteroatom selected from nitrogen and oxygen, wherein the heteroaryl is substituted with m occurrences of R⁵ and n occurrences of R⁶. In certain embodiments, A¹ is 9 membered bicyclic heteroaryl containing 2 heteroatoms independently selected from nitrogen and oxygen, wherein the heteroaryl is substituted with m occurrences of R⁵ and n occurrences of R⁶. In certain embodiments, A¹ is 9 membered bicyclic heteroaryl containing 3 heteroatoms independently selected from nitrogen and oxygen, wherein the heteroaryl is substituted with m occurrences of R⁵ and n occurrences of R⁶. In certain embodiments, A¹ is 10 membered bicyclic heteroaryl containing 1, 2, or 3 heteroatoms independently selected from nitrogen and oxygen, wherein the heteroaryl is substituted with m occurrences of R⁵ and n occurrences of R⁶. In certain embodiments, A¹ is 10 membered bicyclic heteroaryl containing 1 heteroatom selected from nitrogen and oxygen, wherein the heteroaryl is substituted with m occurrences of R⁵ and n occurrences of R⁶. In certain embodiments, A¹ is 10 membered bicyclic heteroaryl containing 2 heteroatoms independently selected from nitrogen and oxygen, wherein the heteroaryl is substituted with m occurrences of R⁵ and n occurrences of R⁶. In certain embodiments, A¹ is 10 membered bicyclic heteroaryl containing 3 heteroatoms independently selected from nitrogen and oxygen, wherein the heteroaryl is substituted with m occurrences of R⁵ and n occurrences of R⁶. In certain embodiments, A¹ is

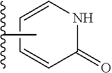

substituted with m occurrences of R⁵ and n occurrences of R⁶. In certain embodiments, A¹ is

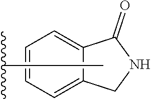

substituted with m occurrences of R⁵ and n occurrences of R⁶. In certain embodiments, A¹ is

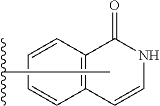

substituted with m occurrences of R⁵ and n occurrences of R⁶. In certain embodiments, A¹ is selected from the groups depicted in the compounds in Table 1 below.

As defined generally above, A² represents independently for each occurrence a 4-6 membered saturated heterocyclyl containing 1 or 2 heteroatoms independently selected from oxygen, nitrogen, and sulfur, wherein the heterocyclyl is substituted with y occurrences of R⁷. In certain embodiments, A² is azetidinyl substituted by hydroxyl. In certain embodiments, A² is a 4 membered saturated heterocyclyl containing 1 or 2 heteroatoms independently selected from oxygen, nitrogen, and sulfur, wherein the heterocyclyl is substituted with y occurrences of R⁷. In certain embodiments, A² is a 4 membered saturated heterocyclyl containing 1 heteroatom selected from oxygen, nitrogen, and sulfur, wherein the heterocyclyl is substituted with y occurrences of R⁷. In certain embodiments, A² is a 4 membered saturated heterocyclyl containing 2 heteroatoms independently selected from oxygen, nitrogen, and sulfur, wherein the heterocyclyl is substituted with y occurrences of R⁷. In certain embodiments, A² is a 5 membered saturated heterocyclyl containing 1 or 2 heteroatoms independently selected from oxygen, nitrogen, and sulfur, wherein the heterocyclyl is substituted with y occurrences of R. In certain embodiments, A² is a 5 membered saturated heterocyclyl containing 1 heteroatom selected from oxygen, nitrogen, and sulfur, wherein the heterocyclyl is substituted with y occurrences of R⁷. In certain embodiments, A² is a 5 membered saturated heterocyclyl containing 2 heteroatoms independently selected from oxygen, nitrogen, and sulfur, wherein the heterocyclyl is substituted with y occurrences of R⁷. In certain embodiments, A² is a 6 membered saturated heterocyclyl containing 1 or 2 heteroatoms independently selected from oxygen, nitrogen, and sulfur, wherein the heterocyclyl is substituted with y occurrences of R⁷. In certain embodiments, A² is a 6 membered saturated heterocyclyl containing 1 heteroatom selected from oxygen, nitrogen, and sulfur, wherein the heterocyclyl is substituted with y occurrences of R⁷. In certain embodiments, A² is a 6 membered saturated heterocyclyl containing 2 heteroatoms independently selected from oxygen, nitrogen, and sulfur, wherein the heterocyclyl is substituted with y occurrences of R⁷. In certain embodiments, A² is selected from the groups depicted in the compounds in Table 1 below.

As defined generally above, R¹ is hydrogen or C₁₋₄ alkyl. In certain embodiments, R¹ is hydrogen. In certain embodiments, R¹ is C₁₋₄ alkyl. In certain embodiments, R¹ is C₁ alkyl. In certain embodiments, R¹ is C₂ alkyl. In certain embodiments, R¹ is C₃ alkyl. In certain embodiments, R¹ is C₄ alkyl. In certain embodiments, R¹ is selected from the groups depicted in the compounds in Table 1 below.

As defined generally above, R² is C₁₋₆ haloalkyl, C₁₋₆ alkyl, C₃₋₇ cycloalkyl, C₁₋₆ alkoxyl, cyano, C₂₋₄ alkenyl, halogen, hydrogen, oxetanyl, tetrahydrofuranyl, or —N(R¹²)(R¹³), wherein the oxetanyl and tetrahydrofuranyl are substituted with 0 or 1 hydroxyl groups. In certain embodiments, R² is C₁₋₆haloalkyl. In certain embodiments, R² is —CF₃. In certain embodiments, R² is C₃₋₇ cycloalkyl. In certain embodiments, R² is cyclopropyl. In certain embodiments, R² is C₁₋₆ alkyl or C₂₋₄ alkenyl. In certain embodiments, R² is halogen, C₁₋₆ alkoxyl, or cyano. In certain embodiments, R² is C₁₋₆ alkyl. In certain embodiments, R² is C₁₋₆ alkoxyl. In certain embodiments, R² is cyano. In certain embodiments, R² is C₂₋₄ alkenyl. In certain embodiments, R² is halogen. In certain embodiments, R² is hydrogen. In certain embodiments, R² is oxetanyl substituted with 0 or 1 hydroxyl groups. In certain embodiments, R² is oxetanyl. In certain embodiments, R² is oxetanyl substituted with 1 hydroxyl group. In certain embodiments, R² is tetrahydrofuranyl substituted with 0 or 1 hydroxyl group. In certain embodiments, R² is tetrahydrofuranyl. In certain embodiments, R² is tetrahydrofuranyl substituted with 1 hydroxyl group. In certain embodiments, R² is —N(R¹²)(R¹³). In certain embodiments, R² is selected from the groups depicted in the compounds in Table 1 below.

As defined generally above, R³ is one of the following: (a) phenyl, a 5-6 membered heteroaryl containing 1, 2, or 3 heteroatoms independently selected from oxygen, nitrogen, and sulfur, a 9-10 membered bicyclic heteroaryl containing 1, 2, or 3 heteroatoms independently selected from oxygen, nitrogen, and sulfur, a 3-10 membered monocyclic or bicyclic saturated or partially unsaturated heterocyclyl containing 1, 2, or 3 heteroatoms independently selected from oxygen, nitrogen, and sulfur,

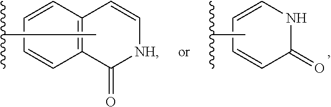

wherein the phenyl, heteroaryl, heterocyclyl,

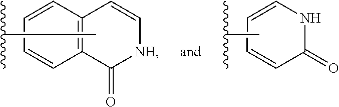

are substituted with t occurrences of R⁴; or (b) C₂₋₆ alkyl, hydroxyl, or —N(R⁹)(R¹⁰). In certain embodiments, R³ is phenyl substituted with t occurrences of R⁴. In certain embodiments, R³ is a 5-6 membered heteroaryl containing 1, 2, or 3 heteroatoms independently selected from oxygen, nitrogen, and sulfur, wherein the heteroaryl is substituted with t occurrences of R⁴. In certain embodiments, R³ is a 9-10 membered bicyclic heteroaryl containing 1, 2, or 3 heteroatoms independently selected from oxygen, nitrogen, and sulfur, wherein the heteroaryl is substituted with t occurrences of R⁴. In certain embodiments, R³ is a 3-10 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁴. In certain embodiments, R³ is tetrahydropyranyl, morpholinyl, piperidinyl, or piperazinyl, each of which is substituted with t occurrences of R⁴. In certain embodiments, R³ is

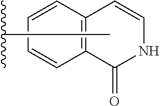

substituted with t occurrences of R⁴. In certain embodiments, R³ is

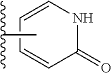

substituted with t occurrences of R⁴. In certain embodiments, R³ is C₃₋₄ alkyl or hydroxyl. In certain embodiments, R³ is —N(R⁹)(R¹⁰).

In certain embodiments, R³ is a 5 membered heteroaryl containing 1, 2, or 3 heteroatoms independently selected from oxygen, nitrogen, and sulfur, wherein the heteroaryl is substituted with t occurrences of R⁴. In certain embodiments, R³ is a 5 membered heteroaryl containing 1 heteroatom selected from oxygen, nitrogen, and sulfur, wherein the heteroaryl is substituted with t occurrences of R⁴. In certain embodiments, R³ is a 5 membered heteroaryl containing 2 heteroatoms independently selected from oxygen, nitrogen, and sulfur, wherein the heteroaryl is substituted with t occurrences of R⁴. In certain embodiments, R³ is a 5 membered heteroaryl containing 3 heteroatoms independently selected from oxygen, nitrogen, and sulfur, wherein the heteroaryl is substituted with t occurrences of R⁴. In certain embodiments, R³ is a 6 membered heteroaryl containing 1, 2, or 3 heteroatoms independently selected from oxygen, nitrogen, and sulfur, wherein the heteroaryl is substituted with t occurrences of R⁴. In certain embodiments, R³ is a 6 membered heteroaryl containing 1 heteroatom selected from oxygen, nitrogen, and sulfur, wherein the heteroaryl is substituted with t occurrences of R⁴. In certain embodiments, R³ is a 6 membered heteroaryl containing 2 heteroatoms independently selected from oxygen, nitrogen, and sulfur, wherein the heteroaryl is substituted with t occurrences of R⁴. In certain embodiments, R³ is a 6 membered heteroaryl containing 3 heteroatoms independently selected from oxygen, nitrogen, and sulfur, wherein the heteroaryl is substituted with t occurrences of R⁴.

In certain embodiments, R³ is a 9 membered bicyclic heteroaryl containing 1, 2, or 3 heteroatoms independently selected from oxygen, nitrogen, and sulfur, wherein the heteroaryl is substituted with t occurrences of R⁴. In certain embodiments, R³ is a 9 membered bicyclic heteroaryl containing 1 heteroatom selected from oxygen, nitrogen, and sulfur, wherein the heteroaryl is substituted with t occurrences of R⁴. In certain embodiments, R³ is a 9 membered bicyclic heteroaryl containing 2 heteroatoms independently selected from oxygen, nitrogen, and sulfur, wherein the heteroaryl is substituted with t occurrences of R⁴. In certain embodiments, R³ is a 9 membered bicyclic heteroaryl containing 3 heteroatoms independently selected from oxygen, nitrogen, and sulfur, wherein the heteroaryl is substituted with t occurrences of R⁴. In certain embodiments, R³ is a 10 membered bicyclic heteroaryl containing 1, 2, or 3 heteroatoms independently selected from oxygen, nitrogen, and sulfur, wherein the heteroaryl is substituted with t occurrences of R⁴. In certain embodiments, R³ is a 10 membered bicyclic heteroaryl containing 1 heteroatom selected from oxygen, nitrogen, and sulfur, wherein the heteroaryl is substituted with t occurrences of R⁴. In certain embodiments, R³ is a 10 membered bicyclic heteroaryl containing 2 heteroatoms independently selected from oxygen, nitrogen, and sulfur, wherein the heteroaryl is substituted with t occurrences of R⁴. In certain embodiments, R³ is a 10 membered bicyclic heteroaryl containing 3 heteroatoms independently selected from oxygen, nitrogen, and sulfur, wherein the heteroaryl is substituted with t occurrences of R⁴.

In certain embodiments, R³ is a 3 membered monocyclic saturated heterocyclyl containing 1 heteroatom selected from oxygen, nitrogen, and sulfur, wherein the heterocyclyl is substituted with t occurrences of R⁴. In certain embodiments, R³ is a 4 membered monocyclic saturated heterocyclyl containing 1 heteroatom selected from oxygen, nitrogen, and sulfur, wherein the heterocyclyl is substituted with t occurrences of R⁴. In certain embodiments, R³ is a 4 membered monocyclic saturated heterocyclyl containing 2 heteroatoms independently selected from oxygen, nitrogen, and sulfur, wherein the heterocyclyl is substituted with t occurrences of R⁴. In certain embodiments, R³ is a 5 membered monocyclic saturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 5 membered monocyclic saturated heterocyclyl containing 1 heteroatom selected from oxygen, nitrogen, and sulfur, wherein the heterocyclyl is substituted with t occurrences of R⁴. In certain embodiments, R³ is a 5 membered monocyclic saturated heterocyclyl containing 2 heteroatoms independently selected from oxygen, nitrogen, and sulfur, wherein the heterocyclyl is substituted with t occurrences of R⁴. In certain embodiments, R³ is a 5 membered monocyclic saturated heterocyclyl containing 3 heteroatoms independently selected from oxygen, nitrogen, and sulfur, wherein the heterocyclyl is substituted with t occurrences of R⁴.

In certain embodiments, R³ is a 6 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 6 membered monocyclic saturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 6 membered monocyclic saturated heterocyclyl containing 1 heteroatom selected from oxygen, nitrogen, and sulfur, wherein the heterocyclyl is substituted with t occurrences of R⁴. In certain embodiments, R³ is a 6 membered monocyclic saturated heterocyclyl containing 2 heteroatoms independently selected from oxygen, nitrogen, and sulfur, wherein the heterocyclyl is substituted with t occurrences of R⁴. In certain embodiments, R³ is a 6 membered monocyclic saturated heterocyclyl containing 3 heteroatoms independently selected from oxygen, nitrogen, and sulfur, wherein the heterocyclyl is substituted with t occurrences of R⁴. In certain embodiments, R³ is a 6 membered monocyclic partially unsaturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 6 membered monocyclic partially unsaturated heterocyclyl containing 1 heteroatom selected from oxygen, nitrogen, and sulfur, wherein the heterocyclyl is substituted with t occurrences of R⁴. In certain embodiments, R³ is a 6 membered monocyclic partially unsaturated heterocyclyl containing 2 heteroatoms independently selected from oxygen, nitrogen, and sulfur, wherein the heterocyclyl is substituted with t occurrences of R⁴. In certain embodiments, R³ is a 6 membered monocyclic partially unsaturated heterocyclyl containing 3 heteroatoms independently selected from oxygen, nitrogen, and sulfur, wherein the heterocyclyl is substituted with t occurrences of R⁴. In certain embodiments, R³ is a 6 membered bicyclic saturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 6 membered bicyclic saturated heterocyclyl containing 1 heteroatom selected from oxygen, nitrogen, and sulfur, wherein the heterocyclyl is substituted with t occurrences of R⁴. In certain embodiments, R³ is a 6 membered bicyclic saturated heterocyclyl containing 2 heteroatoms independently selected from oxygen, nitrogen, and sulfur, wherein the heterocyclyl is substituted with t occurrences of R⁴. In certain embodiments, R³ is a 6 membered bicyclic saturated heterocyclyl containing 3 heteroatoms independently selected from oxygen, nitrogen, and sulfur, wherein the heterocyclyl is substituted with t occurrences of R⁴. In certain embodiments, R³ is a 6 membered bicyclic partially unsaturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 6 membered bicyclic partially unsaturated heterocyclyl containing 1 heteroatom selected from oxygen, nitrogen, and sulfur, wherein the heterocyclyl is substituted with t occurrences of R⁴. In certain embodiments, R³ is a 6 membered bicyclic partially unsaturated heterocyclyl containing 2 heteroatoms independently selected from oxygen, nitrogen, and sulfur, wherein the heterocyclyl is substituted with t occurrences of R⁴. In certain embodiments, R³ is a 6 membered bicyclic partially unsaturated heterocyclyl containing 3 heteroatoms independently selected from oxygen, nitrogen, and sulfur, wherein the heterocyclyl is substituted with t occurrences of R⁴.

In certain embodiments, R³ is a 7 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 7 membered monocyclic saturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 7 membered monocyclic saturated heterocyclyl containing 1 heteroatom selected from oxygen, nitrogen, and sulfur, wherein the heterocyclyl is substituted with t occurrences of R⁴. In certain embodiments, R³ is a 7 membered monocyclic saturated heterocyclyl containing 2 heteroatoms independently selected from oxygen, nitrogen, and sulfur, wherein the heterocyclyl is substituted with t occurrences of R⁴. In certain embodiments, R³ is a 7 membered monocyclic saturated heterocyclyl containing 3 heteroatoms independently selected from oxygen, nitrogen, and sulfur, wherein the heterocyclyl is substituted with t occurrences of R⁴. In certain embodiments, R³ is a 7 membered monocyclic partially unsaturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 7 membered monocyclic partially unsaturated heterocyclyl containing 1 heteroatom selected from oxygen, nitrogen, and sulfur, wherein the heterocyclyl is substituted with t occurrences of R⁴. In certain embodiments, R³ is a 7 membered monocyclic partially unsaturated heterocyclyl containing 2 heteroatoms independently selected from oxygen, nitrogen, and sulfur, wherein the heterocyclyl is substituted with t occurrences of R⁴. In certain embodiments, R³ is a 7 membered monocyclic partially unsaturated heterocyclyl containing 3 heteroatoms independently selected from oxygen, nitrogen, and sulfur, wherein the heterocyclyl is substituted with t occurrences of R⁴. In certain embodiments, R³ is a 7 membered bicyclic saturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 7 membered bicyclic saturated heterocyclyl containing 1 heteroatom selected from oxygen, nitrogen, and sulfur, wherein the heterocyclyl is substituted with t occurrences of R⁴. In certain embodiments, R³ is a 7 membered bicyclic saturated heterocyclyl containing 2 heteroatoms independently selected from oxygen, nitrogen, and sulfur, wherein the heterocyclyl is substituted with t occurrences of R⁴. In certain embodiments, R³ is a 7 membered bicyclic saturated heterocyclyl containing 3 heteroatoms independently selected from oxygen, nitrogen, and sulfur, wherein the heterocyclyl is substituted with t occurrences of R⁴. In certain embodiments, R³ is a 7 membered bicyclic partially unsaturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 7 membered bicyclic partially unsaturated heterocyclyl containing 1 heteroatom selected from oxygen, nitrogen, and sulfur, wherein the heterocyclyl is substituted with t occurrences of R⁴. In certain embodiments, R³ is a 7 membered bicyclic partially unsaturated heterocyclyl containing 2 heteroatoms independently selected from oxygen, nitrogen, and sulfur, wherein the heterocyclyl is substituted with t occurrences of R⁴. In certain embodiments, R³ is a 7 membered bicyclic partially unsaturated heterocyclyl containing 3 heteroatoms independently selected from oxygen, nitrogen, and sulfur, wherein the heterocyclyl is substituted with t occurrences of R⁴.

In certain embodiments, R³ is a 8 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 8 membered monocyclic saturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 8 membered monocyclic saturated heterocyclyl containing 1 heteroatom selected from oxygen, nitrogen, and sulfur, wherein the heterocyclyl is substituted with t occurrences of R⁴. In certain embodiments, R³ is a 8 membered monocyclic saturated heterocyclyl containing 2 heteroatoms independently selected from oxygen, nitrogen, and sulfur, wherein the heterocyclyl is substituted with t occurrences of R⁴. In certain embodiments, R³ is a 8 membered monocyclic saturated heterocyclyl containing 3 heteroatoms independently selected from oxygen, nitrogen, and sulfur, wherein the heterocyclyl is substituted with t occurrences of R⁴. In certain embodiments, R³ is a 8 membered monocyclic partially unsaturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 8 membered monocyclic partially unsaturated heterocyclyl containing 1 heteroatom selected from oxygen, nitrogen, and sulfur, wherein the heterocyclyl is substituted with t occurrences of R⁴. In certain embodiments, R³ is a 8 membered monocyclic partially unsaturated heterocyclyl containing 2 heteroatoms independently selected from oxygen, nitrogen, and sulfur, wherein the heterocyclyl is substituted with t occurrences of R⁴. In certain embodiments, R³ is a 8 membered monocyclic partially unsaturated heterocyclyl containing 3 heteroatoms independently selected from oxygen, nitrogen, and sulfur, wherein the heterocyclyl is substituted with t occurrences of R⁴. In certain embodiments, R³ is a 8 membered bicyclic saturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 8 membered bicyclic saturated heterocyclyl containing 1 heteroatom selected from oxygen, nitrogen, and sulfur, wherein the heterocyclyl is substituted with t occurrences of R⁴. In certain embodiments, R³ is a 8 membered bicyclic saturated heterocyclyl containing 2 heteroatoms independently selected from oxygen, nitrogen, and sulfur, wherein the heterocyclyl is substituted with t occurrences of R⁴. In certain embodiments, R³ is a 8 membered bicyclic saturated heterocyclyl containing 3 heteroatoms independently selected from oxygen, nitrogen, and sulfur, wherein the heterocyclyl is substituted with t occurrences of R⁴. In certain embodiments, R³ is a 8 membered bicyclic partially unsaturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 8 membered bicyclic partially unsaturated heterocyclyl containing 1 heteroatom selected from oxygen, nitrogen, and sulfur, wherein the heterocyclyl is substituted with t occurrences of R⁴. In certain embodiments, R³ is a 8 membered bicyclic partially unsaturated heterocyclyl containing 2 heteroatoms independently selected from oxygen, nitrogen, and sulfur, wherein the heterocyclyl is substituted with t occurrences of R⁴. In certain embodiments, R³ is a 8 membered bicyclic partially unsaturated heterocyclyl containing 3 heteroatoms independently selected from oxygen, nitrogen, and sulfur, wherein the heterocyclyl is substituted with t occurrences of R⁴.

In certain embodiments, R³ is a 9 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 9 membered monocyclic saturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 9 membered monocyclic saturated heterocyclyl containing 1 heteroatom selected from oxygen, nitrogen, and sulfur, wherein the heterocyclyl is substituted with t occurrences of R⁴. In certain embodiments, R³ is a 9 membered monocyclic saturated heterocyclyl containing 2 heteroatoms independently selected from oxygen, nitrogen, and sulfur, wherein the heterocyclyl is substituted with t occurrences of R⁴. In certain embodiments, R³ is a 9 membered monocyclic saturated heterocyclyl containing 3 heteroatoms independently selected from oxygen, nitrogen, and sulfur, wherein the heterocyclyl is substituted with t occurrences of R⁴. In certain embodiments, R³ is a 9 membered monocyclic partially unsaturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 9 membered monocyclic partially unsaturated heterocyclyl containing 1 heteroatom selected from oxygen, nitrogen, and sulfur, wherein the heterocyclyl is substituted with t occurrences of R⁴. In certain embodiments, R³ is a 9 membered monocyclic partially unsaturated heterocyclyl containing 2 heteroatoms independently selected from oxygen, nitrogen, and sulfur, wherein the heterocyclyl is substituted with t occurrences of R⁴. In certain embodiments, R³ is a 9 membered monocyclic partially unsaturated heterocyclyl containing 3 heteroatoms independently selected from oxygen, nitrogen, and sulfur, wherein the heterocyclyl is substituted with t occurrences of R⁴. In certain embodiments, R³ is a 9 membered bicyclic saturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 9 membered bicyclic saturated heterocyclyl containing 1 heteroatom selected from oxygen, nitrogen, and sulfur, wherein the heterocyclyl is substituted with t occurrences of R⁴. In certain embodiments, R³ is a 9 membered bicyclic saturated heterocyclyl containing 2 heteroatoms independently selected from oxygen, nitrogen, and sulfur, wherein the heterocyclyl is substituted with t occurrences of R⁴. In certain embodiments, R³ is a 9 membered bicyclic saturated heterocyclyl containing 3 heteroatoms independently selected from oxygen, nitrogen, and sulfur, wherein the heterocyclyl is substituted with t occurrences of R⁴. In certain embodiments, R³ is a 9 membered bicyclic partially unsaturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 9 membered bicyclic partially unsaturated heterocyclyl containing 1 heteroatom selected from oxygen, nitrogen, and sulfur, wherein the heterocyclyl is substituted with t occurrences of R⁴. In certain embodiments, R³ is a 9 membered bicyclic partially unsaturated heterocyclyl containing 2 heteroatoms independently selected from oxygen, nitrogen, and sulfur, wherein the heterocyclyl is substituted with t occurrences of R⁴. In certain embodiments, R³ is a 9 membered bicyclic partially unsaturated heterocyclyl containing 3 heteroatoms independently selected from oxygen, nitrogen, and sulfur, wherein the heterocyclyl is substituted with t occurrences of R⁴.

In certain embodiments, R³ is a 10 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 10 membered monocyclic saturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 10 membered monocyclic saturated heterocyclyl containing 1 heteroatom selected from oxygen, nitrogen, and sulfur, wherein the heterocyclyl is substituted with t occurrences of R⁴. In certain embodiments, R³ is a 10 membered monocyclic saturated heterocyclyl containing 2 heteroatoms independently selected from oxygen, nitrogen, and sulfur, wherein the heterocyclyl is substituted with t occurrences of R. In certain embodiments, R³ is a 10 membered monocyclic saturated heterocyclyl containing 3 heteroatoms independently selected from oxygen, nitrogen, and sulfur, wherein the heterocyclyl is substituted with t occurrences of R⁴. In certain embodiments, R³ is a 10 membered monocyclic partially unsaturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 10 membered monocyclic partially unsaturated heterocyclyl containing 1 heteroatom selected from oxygen, nitrogen, and sulfur, wherein the heterocyclyl is substituted with t occurrences of R⁴. In certain embodiments, R³ is a 10 membered monocyclic partially unsaturated heterocyclyl containing 2 heteroatoms independently selected from oxygen, nitrogen, and sulfur, wherein the heterocyclyl is substituted with t occurrences of R⁴. In certain embodiments, R³ is a 10 membered monocyclic partially unsaturated heterocyclyl containing 3 heteroatoms independently selected from oxygen, nitrogen, and sulfur, wherein the heterocyclyl is substituted with t occurrences of R⁴. In certain embodiments, R³ is a 10 membered bicyclic saturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 10 membered bicyclic saturated heterocyclyl containing 1 heteroatom selected from oxygen, nitrogen, and sulfur, wherein the heterocyclyl is substituted with t occurrences of R⁴. In certain embodiments, R³ is a 10 membered bicyclic saturated heterocyclyl containing 2 heteroatoms independently selected from oxygen, nitrogen, and sulfur, wherein the heterocyclyl is substituted with t occurrences of R⁴. In certain embodiments, R³ is a 10 membered bicyclic saturated heterocyclyl containing 3 heteroatoms independently selected from oxygen, nitrogen, and sulfur, wherein the heterocyclyl is substituted with t occurrences of R⁴. In certain embodiments, R³ is a 10 membered bicyclic partially unsaturated heterocyclyl containing 1, 2, or 3 heteroatoms independently selected from oxygen, nitrogen, and sulfur, wherein the heterocyclyl is substituted with t occurrences of R⁴. In certain embodiments, R³ is a 10 membered bicyclic partially unsaturated heterocyclyl containing 1 heteroatom selected from oxygen, nitrogen, and sulfur, wherein the heterocyclyl is substituted with t occurrences of R⁴. In certain embodiments, R³ is a 10 membered bicyclic partially unsaturated heterocyclyl containing 2 heteroatoms independently selected from oxygen, nitrogen, and sulfur, wherein the heterocyclyl is substituted with t occurrences of R⁴. In certain embodiments, R³ is a 10 membered bicyclic partially unsaturated heterocyclyl containing 3 heteroatoms independently selected from oxygen, nitrogen, and sulfur, wherein the heterocyclyl is substituted with t occurrences of R⁴.

In certain embodiments, R³ is C₂₋₆ alkyl. In certain embodiments, R³ is hydroxyl. In certain embodiments, R³ is tetrahydropyranyl substituted with t occurrences of R⁴. In certain embodiments, R³ is morpholinyl substituted with t occurrences of R⁴. In certain embodiments, R³ is piperidinyl substituted with t occurrences of R⁴. In certain embodiments, R³ is piperazinyl substituted with t occurrences of R⁴. In certain embodiments, R³ is selected from the groups depicted in the compounds in Table 1 below.

As defined generally above, R⁴ represents independently for each occurrence halo, hydroxyl, C₁₋₆ alkyl, C₁₋₆ haloalkyl, C₁₋₆ hydroxyalkyl, C₁₋₆ alkoxyl, C₃₋₇ cycloalkyl, —C(O)R¹¹, —C(O)N(R⁹)(R¹), or —N(R⁹)C(O)R¹¹. In certain embodiments, R⁴ represents independently for each occurrence halo, hydroxyl, C₁₋₆ alkyl, C₁₋₆ haloalkyl, or C₃₋₇ cycloalkyl. In certain embodiments, R⁴ represents independently for each occurrence halo or C₁₋₆ alkyl. In certain embodiments, R⁴ is halo. In certain embodiments, R⁴ is hydroxyl. In certain embodiments, R⁴ is C₁₋₆ alkyl. In certain embodiments, R⁴ is C₁₋₆ haloalkyl. In certain embodiments, R⁴ is C₁₋₆ hydroxyalkyl. In certain embodiments, R⁴ is C₁₋₆ alkoxyl. In certain embodiments, R⁴ is C₃₋₇ cycloalkyl. In certain embodiments, R⁴ is —C(O)R¹¹. In certain embodiments, R⁴ is —C(O)N(R⁹)(R¹⁰). In certain embodiments, R⁴ is —N(R⁹)C(O)R¹¹. In certain embodiments, R⁴ is selected from the groups depicted in the compounds in Table 1 below.

As defined generally above, R⁵ is a 5-6 membered heteroaryl containing 1, 2, or 3 heteroatoms independently selected from oxygen, nitrogen, and sulfur, or a 3-7 membered saturated heterocyclyl containing 1, 2, or 3 heteroatoms independently selected from oxygen, nitrogen, and sulfur, wherein the heteroaryl and heterocyclyl are substituted with q occurrences of R⁷. In certain embodiments, R⁵ is a 5 membered heteroaryl containing 1, 2, or 3 heteroatoms independently selected from oxygen, nitrogen, and sulfur, wherein the heteroaryl is substituted with q occurrences of R⁷. In certain embodiments, R⁵ is a 1,2,3-triazolyl, pyrazolyl, oxazolyl, imidazolyl, isoxazolyl, pyrrolyl, or furanyl, each of which substituted with q occurrences of R⁷. In certain embodiments, R⁵ is 1,2,3-triazolyl substituted with q occurrences of R⁷. In certain embodiments, R⁵ is a 5 membered heteroaryl containing 1 heteroatom selected from oxygen, nitrogen, and sulfur, wherein the heteroaryl is substituted with q occurrences of R⁷. In certain embodiments, R^{•} is a 5 membered heteroaryl containing 2 heteroatoms independently selected from oxygen, nitrogen, and sulfur, wherein the heteroaryl is substituted with q occurrences of R⁷. In certain embodiments, R⁵ is a 5 membered heteroaryl containing 3 heteroatoms independently selected from oxygen, nitrogen, and sulfur, wherein the heteroaryl is substituted with q occurrences of R^{•}. In certain embodiments, R⁵ is a 6 membered heteroaryl containing 1, 2, or 3 heteroatoms independently selected from oxygen, nitrogen, and sulfur, wherein the heteroaryl is substituted with q occurrences of R⁷. In certain embodiments, R⁵ is a 6 membered heteroaryl containing 1 heteroatom selected from oxygen, nitrogen, and sulfur, wherein the heteroaryl is substituted with q occurrences of R⁷. In certain embodiments, R⁵ is a 6 membered heteroaryl containing 2 heteroatoms independently selected from oxygen, nitrogen, and sulfur, wherein the heteroaryl is substituted with q occurrences of R⁷. In certain embodiments, R⁵ is a 6 membered heteroaryl containing 3 heteroatoms independently selected from oxygen, nitrogen, and sulfur, wherein the heteroaryl is substituted with q occurrences of R⁷. In certain embodiments, R⁵ is a pyrazolyl substituted with q occurrences of R⁷. In certain embodiments, R⁵ is an oxazolyl substituted with q occurrences of R⁷. In certain embodiments, R⁵ is an imidazolyl substituted with q occurrences of R⁷. In certain embodiments, R⁵ is an isoxazolyl substituted with q occurrences of R⁷. In certain embodiments, R⁵ is a pyrrolyl substituted with q occurrences of R⁷. In certain embodiments, R⁵ is a furanyl substituted with q occurrences of R⁷. In certain embodiments, R⁵ is selected from the groups depicted in the compounds in Table 1 below.

As defined generally above, R⁶ represents independently for each occurrence halo, hydroxyl, C₁₋₆ alkyl. C₁₋₆ haloalkyl, C₁₋₆ hydroxyalkyl, C₁₋₆ alkoxyl, C₃₋₇ cycloalkyl, cyano, —O—C₃₋₇ cycloalkyl, —N(R⁹)(R¹⁰), —(C₀₋₄ alkylene)-C(O)R⁸, —C(O)N(R⁹)(R¹⁰), or —N(R⁹)C(O)R¹. In certain embodiments, R⁶ represents independently for each occurrence halo, C₁₋₆ alkyl, C₁₋₆ haloalkyl, cyano, or —(C₀₋₄ alkylene)-C(O)R⁶. In certain embodiments, R⁶ represents independently for each occurrence halo, cyano, or —(C₀₋₄ alkylene)-C(O)R⁸. In certain embodiments, R⁶ is chloro. In certain embodiments, R⁶ is C₁₋₆ haloalkyl. In certain embodiments, R⁶ is —CF₃. In certain embodiments, R⁶ is halo. In certain embodiments, R⁶ is Br. In certain embodiments, R⁶ is F. In certain embodiments, R⁶ is I. In certain embodiments, R⁶ is hydroxyl. In certain embodiments, R⁶ is C₁₋₆ alkyl. In certain embodiments, R⁶ is C₁₋₆ hydroxyalkyl. In certain embodiments, R⁶ is C₁₋₆ alkoxyl. In certain embodiments, R⁶ is C₃₋₇ cycloalkyl. In certain embodiments, R⁶ is cyano. In certain embodiments, R⁶ is —O—C₃₋₇ cycloalkyl. In certain embodiments, R⁶ is —N(R⁹)(R¹⁰). In certain embodiments, R⁶ is —(C₀₋₄ alkylene)-C(O)R⁶. In certain embodiments, R⁶ is —C(O)N(R⁹)(R¹⁰). In certain embodiments, R⁶ is —N(R⁹)C(O)R¹¹. In certain embodiments, R⁶ is selected from the groups depicted in the compounds in Table 1 below.

As defined generally above, R⁷ represents independently for each occurrence halo, hydroxyl, C₁₋₆ alkyl, C₁₋₆ haloalkyl, C₁₋₆ alkoxyl, or C₃₋₇ cycloalkyl. In certain embodiments, R⁷ is halo. In certain embodiments, R⁷ is hydroxyl. In certain embodiments, R⁷ is C₁₋₆ alkyl. In certain embodiments, R⁷ is C₁₋₆ haloalkyl. In certain embodiments, R⁷ is C₁₋₆ alkoxyl. In certain embodiments, R⁷ is C₃₋₇ cycloalkyl. In certain embodiments, R⁷ is selected from the groups depicted in the compounds in Table 1 below.

As defined generally above, R⁸ is —OH, —O—(C₁₋₆ alkyl), —O—C₃₋₇ cycloalkyl, or A². In certain embodiments, R⁸ is —OH. In certain embodiments, R⁸ is —O—(C₁₋₆ alkyl). In certain embodiments, R⁸ is —O—C₃₋₇ cycloalkyl. In certain embodiments, R⁸ is A². In certain embodiments, R^{○} is selected from the groups depicted in the compounds in Table 1 below.

As defined generally above, R⁹ and R¹⁰ are independently hydrogen, C₁₋₆ alkyl, C₃₋₇ cycloalkyl, C₂₋₄ hydroxyalkyl, or —(C₂₋₆ alkylene)-(C₁₋₆ alkoxyl), or R⁹ and R¹⁰ are taken together with the nitrogen atom to which they are attached to form a 3-7 membered heterocyclic ring containing 1 nitrogen atom, wherein the heterocyclic ring is substituted with 0 or 1 groups independently selected from hydroxyl, halo, and C₁₋₆ alkyl. In certain embodiments, R⁹ is hydrogen. In certain embodiments, R⁹ is C₁₋₆ alkyl. In certain embodiments, R⁹ is C₃₋₇ cycloalkyl. In certain embodiments, R⁹ is C₂₋₄hydroxyalkyl. In certain embodiments, R⁹ is —(C₂₋₆ alkylene)-(C₁₋₆ alkoxyl). In certain embodiments, R¹⁰ is hydrogen. In certain embodiments, R¹⁰ is C₁₋₆ alkyl. In certain embodiments, R¹⁰ is C₃₋₇ cycloalkyl. In certain embodiments, R¹⁰ is C₂₋₄ hydroxyalkyl. In certain embodiments, R¹⁰ is —(C₂₋₆ alkylene)-(C₁₋₆ alkoxyl). In certain embodiments, R⁹ and R¹⁰ are taken together with the nitrogen atom to which they are attached to form a 3-7 membered heterocyclic ring containing 1 nitrogen atom, wherein the heterocyclic ring is substituted with 0 or 1 groups independently selected from hydroxyl, halo, and C₁₋₆ alkyl. In certain embodiments, R⁹ is selected from the groups depicted in the compounds in Table 1 below. In certain embodiments, R¹⁰ is selected from the groups depicted in the compounds in Table 1 below.

As defined generally above, R¹¹ represents independently for each occurrence C₁₋₆ alkyl or (Co-s alkylene)-C₃₋₇ cycloalkyl. In certain embodiments, R¹¹ is C₁₋₆ alkyl. In certain embodiments, R¹¹ is C₁ alkyl. In certain embodiments, R¹¹ is C₂ alkyl. In certain embodiments, R¹¹ is C₃ alkyl. In certain embodiments, R¹¹ is C₄ alkyl. In certain embodiments, R¹¹ is C₅ alkyl. In certain embodiments, R¹¹ is C₆ alkyl. In certain embodiments, R¹¹ is (C₀₋₅ alkylene)-C₃₋₇ cycloalkyl. In certain embodiments, R¹¹ is selected from the groups depicted in the compounds in Table 1 below.

As defined generally above, R¹² and R¹³ are independently hydrogen, C₁₋₆ alkyl, or C₃₋₅ cycloalkyl, or R¹² and R¹³ are taken together with the nitrogen atom to which they are attached to form a 3-7 membered heterocyclic ring containing 1 nitrogen atom. In certain embodiments, R¹² is hydrogen. In certain embodiments, R¹² is C₁₋₆ alkyl. In certain embodiments, R¹² is C₁ alkyl. In certain embodiments, R¹² is C₂ alkyl. In certain embodiments, R¹² is C₃ alkyl. In certain embodiments, R¹² is C₄ alkyl. In certain embodiments, R¹² is C₅ alkyl. In certain embodiments, R¹² is C₆ alkyl. In certain embodiments, R¹² is C₃₋₅ cycloalkyl. In certain embodiments, R¹² is C₃ cycloalkyl. In certain embodiments, R¹² is C₄ cycloalkyl. In certain embodiments, R¹² is C₅ cycloalkyl. In certain embodiments, R¹³ is hydrogen. In certain embodiments, R¹³ is C₁₋₆ alkyl. In certain embodiments, R¹³ is C₁ alkyl. In certain embodiments, R¹³ is C₂ alkyl. In certain embodiments, R¹³ is C₃ alkyl. In certain embodiments, R¹³ is C₄ alkyl. In certain embodiments, R¹³ is C₅ alkyl. In certain embodiments, R¹³ is C₆ alkyl. In certain embodiments, R¹³ is C₃₋₅ cycloalkyl. In certain embodiments, R¹³ is C₃ cycloalkyl. In certain embodiments, R¹³ is C₄ cycloalkyl. In certain embodiments, R¹³ is C₅ cycloalkyl. In certain embodiments, R¹² and R¹³ are taken together with the nitrogen atom to which they are attached to form a 3-7 membered heterocyclic ring containing 1 nitrogen atom. In certain embodiments, R¹² and R¹³ are taken together with the nitrogen atom to which they are attached to form a 3 membered heterocyclic ring containing 1 nitrogen atom. In certain embodiments, R¹² and R¹³ are taken together with the nitrogen atom to which they are attached to form a 4 membered heterocyclic ring containing 1 nitrogen atom. In certain embodiments, R¹² and R¹³ are taken together with the nitrogen atom to which they are attached to form a 5 membered heterocyclic ring containing 1 nitrogen atom. In certain embodiments, R¹² and R¹³ are taken together with the nitrogen atom to which they are attached to form a 6 membered heterocyclic ring containing 1 nitrogen atom. In certain embodiments, R¹² and R¹³ are taken together with the nitrogen atom to which they are attached to form a 7 membered heterocyclic ring containing 1 nitrogen atom. In certain embodiments, R¹² is selected from the groups depicted in the compounds in Table 1 below. In certain embodiments, R¹³ is selected from the groups depicted in the compounds in Table 1 below.

As defined generally above, m is 0 or 1. In certain embodiments, m is 1. In certain embodiments, m is 0. In certain embodiments, m is selected from the corresponding value in the groups depicted in the compounds in Table 1 below.

As defined generally above, n, q, t, and y are independently 0, 1, or 2. In certain embodiments, n is 1. In certain embodiments, n is 0. In certain embodiments, q is 0. In certain embodiments, t is 1. In certain embodiments, t is 0. In certain embodiments, n is 2. In certain embodiments, q is 1. In certain embodiments, q is 2. In certain embodiments, t is 2. In certain embodiments, y is 0. In certain embodiments, y is 1. In certain embodiments, y is 2. In certain embodiments, n is selected from the corresponding value in the groups depicted in the compounds in Table 1 below. In certain embodiments, q is selected from the corresponding value in the groups depicted in the compounds in Table 1 below. In certain embodiments, t is selected from the corresponding value in the groups depicted in the compounds in Table 1 below. In certain embodiments, y is selected from the corresponding value in the groups depicted in the compounds in Table 1 below.

The description above describes multiple embodiments relating to compounds of Formula I-1. The patent application specifically contemplates all combinations of the embodiments.

In certain embodiments, the compound of Formula I-1 is further defined by Formula Ia-1 or a pharmaceutically acceptable salt thereof:

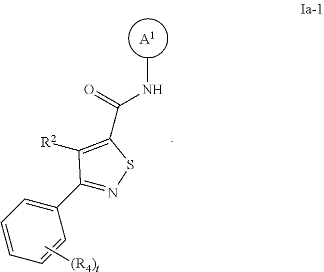

In certain embodiments, the definition of variables R², R⁴, A¹, and t is one of the embodiments described above in connection with Formula I-1.

The description above describes multiple embodiments relating to compounds of Formula Ia-1. The patent application specifically contemplates all combinations of the embodiments.

In certain embodiments, the compound of Formula I-1 is further defined by Formula Ib-1 or Ic-1 or a pharmaceutically acceptable salt thereof:

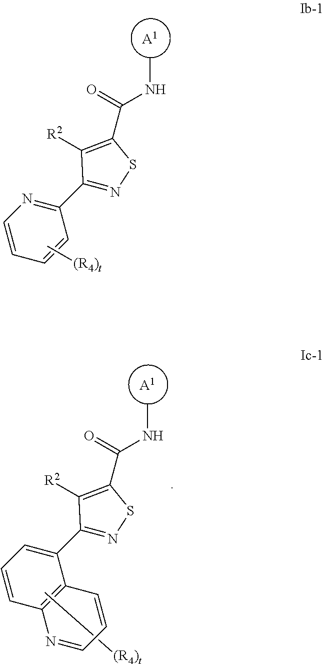

In certain embodiments, the definition of variables R², R⁴, A¹, and t is one of the embodiments described above in connection with Formula I-1. In certain embodiments, the compound of Formula I-1 is further defined by Formula Ib-1 or a pharmaceutically acceptable salt thereof. In certain embodiments, the compound of Formula I-1 is further defined by Formula Ic-1 or a pharmaceutically acceptable salt thereof.

The description above describes multiple embodiments relating to compounds of Formulae Ib-1 and Ic-1. The patent application specifically contemplates all combinations of the embodiments.

In certain embodiments, the compound of Formula I-1 is further defined by Formula Id-1 or a pharmaceutically acceptable salt thereof:

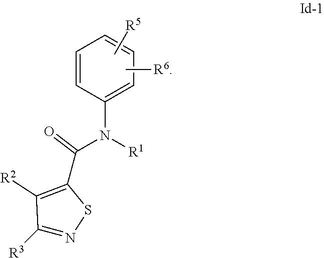

In certain embodiments, the definition of variables R¹, R², R³, R⁵, and R⁶ is one of the embodiments described above in connection with Formula I-1.

The description above describes multiple embodiments relating to compounds of Formula Id-1. The patent application specifically contemplates all combinations of the embodiments.

In certain embodiments, the compound of Formula I-1 is further defined by Formula Ie-1 or a pharmaceutically acceptable salt thereof:

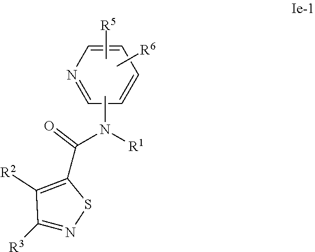

In certain embodiments, the definition of variables R¹, R², R³, R⁵, and R⁶ is one of the embodiments described above in connection with Formula I-1.

The description above describes multiple embodiments relating to compounds of Formula Ie-1. The patent application specifically contemplates all combinations of the embodiments.

In certain embodiments, the compound of Formula I-1 is further defined by Formula If-1 or a pharmaceutically acceptable salt thereof:

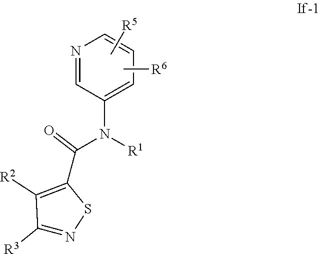

In certain embodiments, the definition of variables R¹, R², R³, R⁵, and R⁶ is one of the embodiments described above in connection with Formula I-1.

The description above describes multiple embodiments relating to compounds of Formula If-1. The patent application specifically contemplates all combinations of the embodiments.

In certain embodiments, the compound of Formula I-1 is further defined by Formula Ig-1 or Ih-1 or a pharmaceutically acceptable salt thereof:

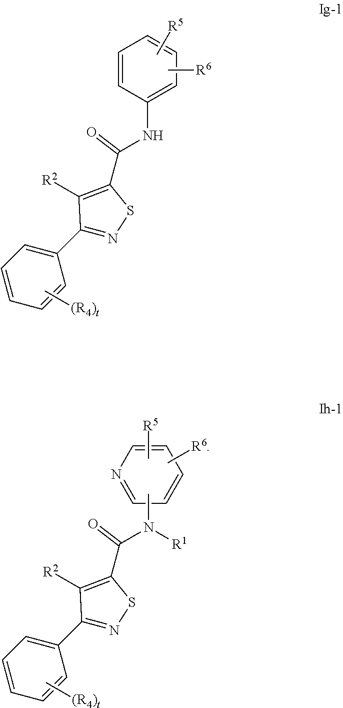

In certain embodiments, the definition of variables R², R⁴, R⁵, R⁶, and t is one of the embodiments described above in connection with Formula I-1. In certain embodiments, the compound of Formula I-1 is further defined by Formula Ig-1 or a pharmaceutically acceptable salt thereof. In certain embodiments, the compound of Formula I-1 is further defined by Formula Ih-1 or a pharmaceutically acceptable salt thereof.

The description above describes multiple embodiments relating to compounds of Formulae Ig-1 and Ih-1. The patent application specifically contemplates all combinations of the embodiments.

In certain embodiments, the compound of Formula I-1 is further defined by Formula II-1, Ij-1, Ik-1, or Il-1 or a pharmaceutically acceptable salt thereof.

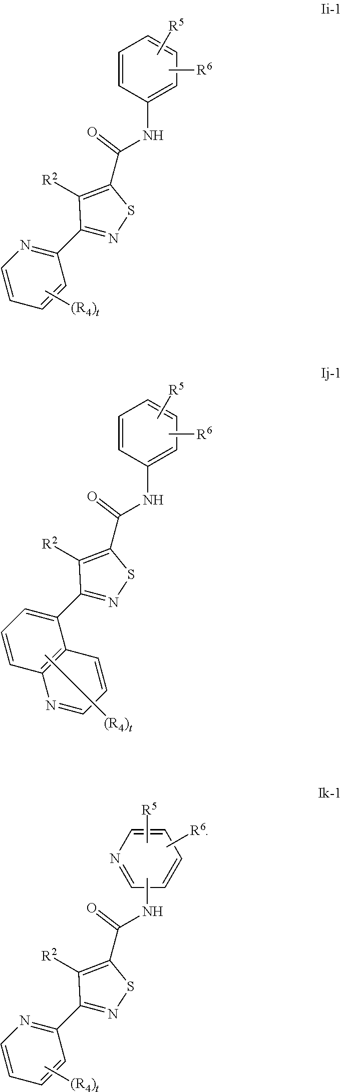

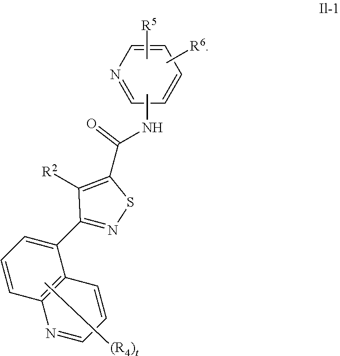

In certain embodiments, the definition of variables R², R⁴, R⁵, R⁶, and t is one of the embodiments described above in connection with Formula I-1. In certain embodiments, the compound of Formula I-1 is further defined by Formula Ii-1 or a pharmaceutically acceptable salt thereof. In certain embodiments, the compound of Formula I-1 is further defined by Formula Ij-1 or a pharmaceutically acceptable salt thereof. In certain embodiments, the compound of Formula I-1 is further defined by Formula Ik-1 or a pharmaceutically acceptable salt thereof. In certain embodiments, the compound of Formula I-1 is further defined by Formula Ii-1 or a pharmaceutically acceptable salt thereof.

The description above describes multiple embodiments relating to compounds of Formulae Ii-1, Ij-1, Ik-1, and Il-1. The patent application specifically contemplates all combinations of the embodiments.

In certain embodiments, the compound of Formula I-1 is further defined by Formula Im-1 or In-1 or a pharmaceutically acceptable salt thereof:

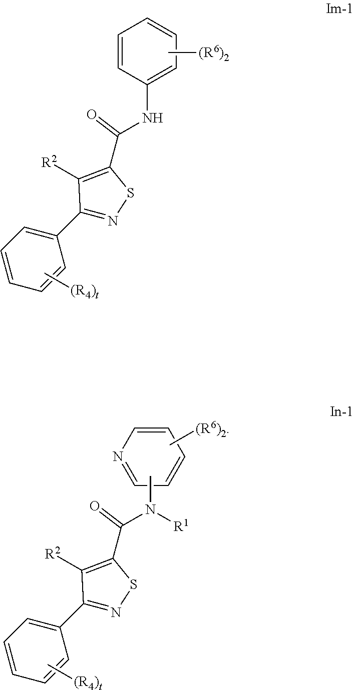

In certain embodiments, the definition of variables R², R⁴, R⁶, and t is one of the embodiments described above in connection with Formula I-1. In certain embodiments, the compound of Formula I-1 is further defined by Formula Im-1 or a pharmaceutically acceptable salt thereof. In certain embodiments, the compound of Formula I-1 is further defined by Formula In-1 or a pharmaceutically acceptable salt thereof.

The description above describes multiple embodiments relating to compounds of Formulae Im-1 and In-1. The patent application specifically contemplates all combinations of the embodiments.

In certain embodiments, the compound of Formula I-1 is further defined by Formula Io-1, Ip-1, Iq-1, or Ir-1 or a pharmaceutically acceptable salt thereof:

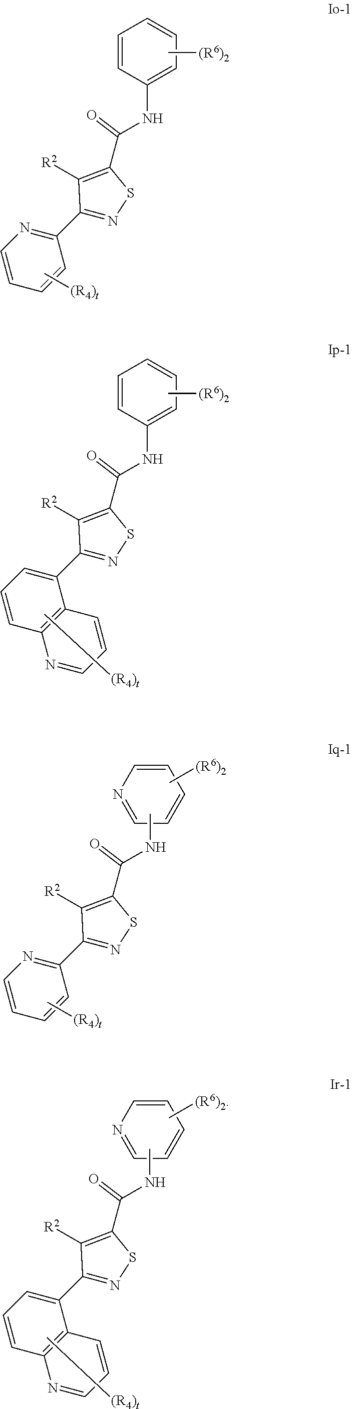

In certain embodiments, the definition of variables R², R⁴, R⁶, and t is one of the embodiments described above in connection with Formula I-1. In certain embodiments, the compound of Formula I-1 is further defined by Formula Io-1 or a pharmaceutically acceptable salt thereof. In certain embodiments, the compound of Formula I-1 is further defined by Formula Ip-1 or a pharmaceutically acceptable salt thereof. In certain embodiments, the compound of Formula I-1 is further defined by Formula Iq-1 or a pharmaceutically acceptable salt thereof. In certain embodiments, the compound of Formula I-1 is further defined by Formula Ir-1 or a pharmaceutically acceptable salt thereof.

The description above describes multiple embodiments relating to compounds of Formulae Io-1, Ip-1, Iq-1, and Ir-1. The patent application specifically contemplates all combinations of the embodiments.

In certain embodiments, the compound of Formula I-1 is further defined by Formula Is-1 or It-1 or a pharmaceutically acceptable salt thereof.

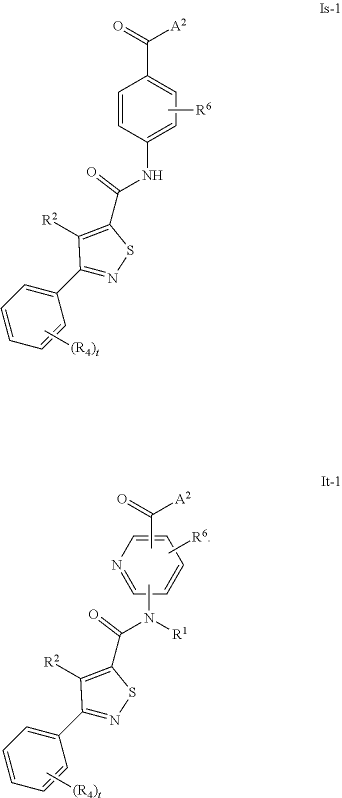

In certain embodiments, the definition of variables R¹, R², R⁴, R⁶, A², and t is one of the embodiments described above in connection with Formula I-1. In certain embodiments, the compound of Formula I-1 is further defined by Formula Is-1 or a pharmaceutically acceptable salt thereof. In certain embodiments, the compound of Formula I-1 is further defined by Formula It-1 or a pharmaceutically acceptable salt thereof.

The description above describes multiple embodiments relating to compounds of Formulae Is-1 and It-1. The patent application specifically contemplates all combinations of the embodiments.

In certain embodiments, the compound of Formula I-1 is further defined by Formula Iu-1, Iv-1, Iw-1, or Ix-1 or a pharmaceutically acceptable salt thereof:

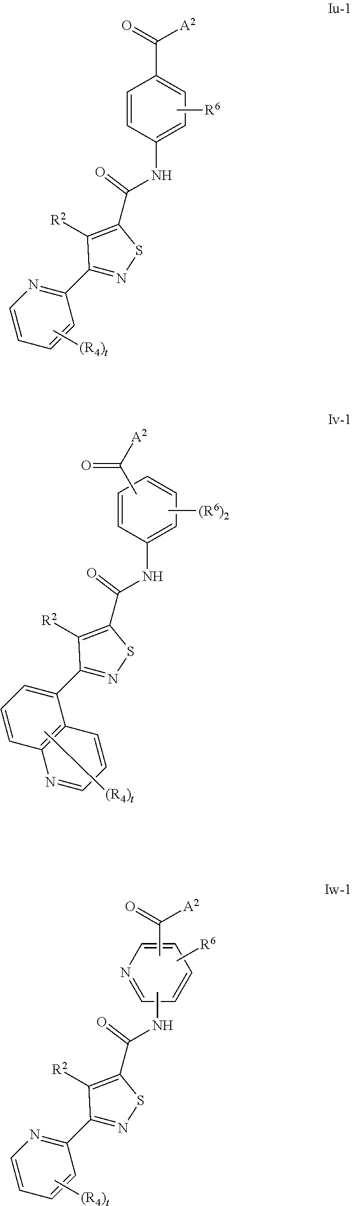

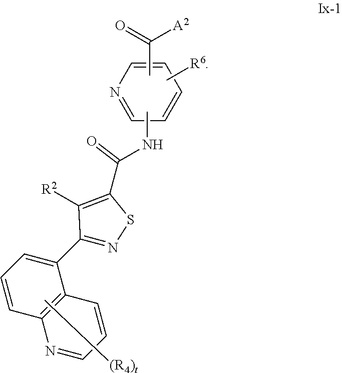

In certain embodiments, the definition of variables R², R⁴, R⁶, A², and t is one of the embodiments described above in connection with Formula I-1. In certain embodiments, the compound of Formula I-1 is further defined by Formula Iu-1 or a pharmaceutically acceptable salt thereof. In certain embodiments, the compound of Formula I-1 is further defined by Formula Iv-1 or a pharmaceutically acceptable salt thereof. In certain embodiments, the compound of Formula I-1 is further defined by Formula Iw-1 or a pharmaceutically acceptable salt thereof. In certain embodiments, the compound of Formula I-1 is further defined by Formula Ix-1 or a pharmaceutically acceptable salt thereof.

The description above describes multiple embodiments relating to compounds of Formulae Iu-1, Iv-1, Iw-1, and Ix-1. The patent application specifically contemplates all combinations of the embodiments.

Another aspect of the invention provides a compound in Table 1 below, or a pharmaceutically acceptable salt thereof. In certain embodiments, the compound is a compound in Table 1. In certain embodiments, the compound is any one of compounds I-1 to I-127 in Table 1. In certain embodiments, the compound is any one of compounds I-1 to I-127 in Table 1, or a pharmaceutically acceptable salt thereof.

TABLE 1

| Compound No. | Structure |
|---|---|
| I-1 | 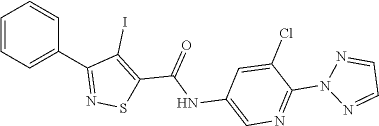 [SMILES: O=C(Nc1cnc(-n2nccn2)c(Cl)c1)c1snc(-c2ccccc2)c1I] |
| I-2 | 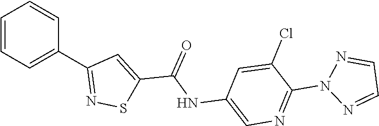 [SMILES: O=C(Nc1cnc(-n2nccn2)c(Cl)c1)c1cc(-c2ccccc2)ns1] |
| I-3 | 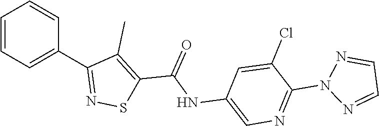 [SMILES: Cc1c(-c2ccccc2)nsc1C(=O)Nc1cnc(-n2nccn2)c(Cl)c1] |
| I-4 | 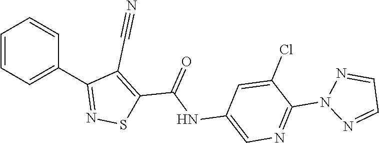 [SMILES: N#Cc1c(-c2ccccc2)nsc1C(=O)Nc1cnc(-n2nccn2)c(Cl)c1] |
| I-5 | 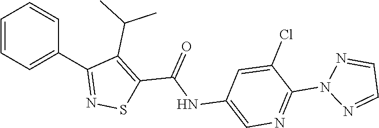 [SMILES: CC(C)c1c(-c2ccccc2)nsc1C(=O)Nc1cnc(-n2nccn2)c(Cl)c1] |
| I-6 | 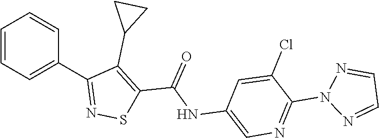 [SMILES: O=C(Nc1cnc(-n2nccn2)c(Cl)c1)c1snc(-c2ccccc2)c1C1CC1] |
| I-7 | 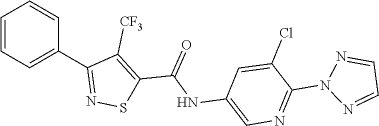 [SMILES: O=C(Nc1cnc(-n2nccn2)c(Cl)c1)c1snc(-c2ccccc2)c1C(F)(F)F] |
| I-8 | 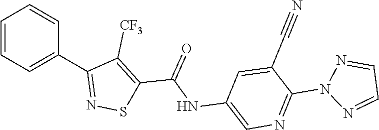 [SMILES: N#Cc1cc(NC(=O)c2snc(-c3ccccc3)c2C(F)(F)F)cnc1-n1nccn1] |
| I-9 | 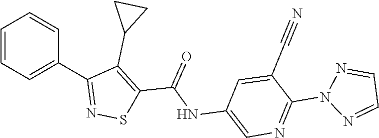 [SMILES: N#Cc1cc(NC(=O)c2snc(-c3ccccc3)c2C2CC2)cnc1-n1nccn1] |
| I-10 | 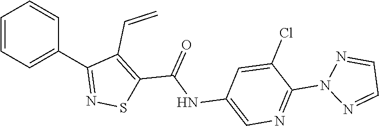 [SMILES: C=Cc1c(-c2ccccc2)nsc1C(=O)Nc1cnc(-n2nccn2)c(Cl)c1] |
| I-11 | 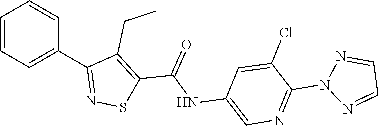 [SMILES: CCc1c(-c2ccccc2)nsc1C(=O)Nc1cnc(-n2nccn2)c(Cl)c1] |
| I-12 | 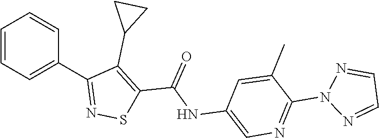 [SMILES: Cc1cc(NC(=O)c2snc(-c3ccccc3)c2C2CC2)cnc1-n1nccn1] |
| I-13 | 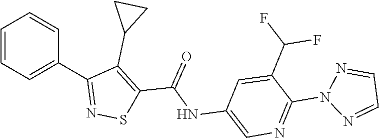 [SMILES: O=C(Nc1cnc(-n2nccn2)c(C(F)F)c1)c1snc(-c2ccccc2)c1C1CC1] |
| I-14 | 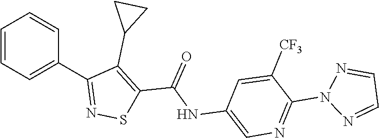 [SMILES: O=C(Nc1cnc(-n2nccn2)c(C(F)(F)F)c1)c1snc(-c2ccccc2)c1C1CC1] |
| I-15 | 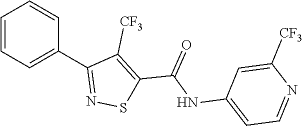 [SMILES: O=C(Nc1ccnc(C(F)(F)F)c1)c1snc(-c2ccccc2)c1C(F)(F)F] |
| I-16 | 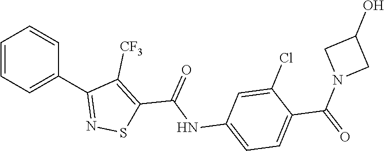 [SMILES: O=C(Nc1ccc(C(=O)N2CC(O)C2)c(Cl)c1)c1snc(-c2ccccc2)c1C(F)(F)F] |
| I-17 | 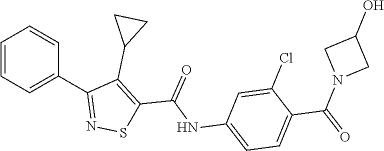 [SMILES: O=C(Nc1ccc(C(=O)N2CC(O)C2)c(Cl)c1)c1snc(-c2ccccc2)c1C1CC1] |
| I-18 | 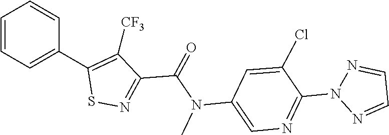 [SMILES: CN(C(=O)c1nsc(-c2ccccc2)c1C(F)(F)F)c1cnc(-n2nccn2)c(Cl)c1] |
| I-19 | 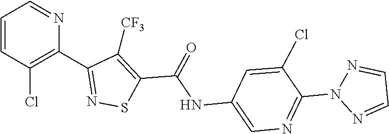 [SMILES: O=C(Nc1cnc(-n2nccn2)c(Cl)c1)c1snc(-c2ncccc2Cl)c1C(F)(F)F] |
| I-20 | 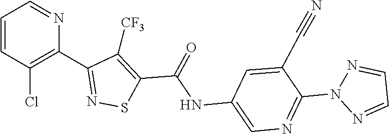 [SMILES: N#Cc1cc(NC(=O)c2snc(-c3ncccc3Cl)c2C(F)(F)F)cnc1-n1nccn1] |
| I-21 | 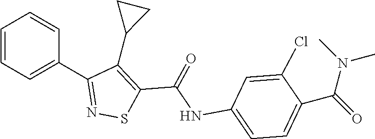 [SMILES: CN(C)C(=O)c1ccc(NC(=O)c2snc(-c3ccccc3)c2C2CC2)cc1Cl] |
| I-22 | 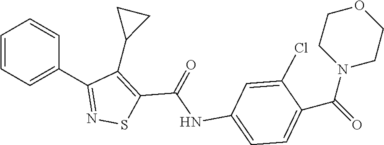 [SMILES: O=C(Nc1ccc(C(=O)N2CCOCC2)c(Cl)c1)c1snc(-c2ccccc2)c1C1CC1] |
| I-23 | 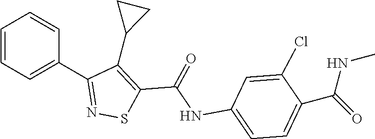 [SMILES: CNC(=O)c1ccc(NC(=O)c2snc(-c3ccccc3)c2C2CC2)cc1Cl] |
| I-24 | 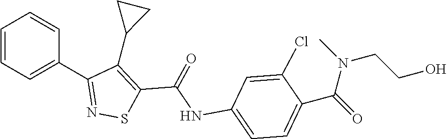 [SMILES: CN(CCO)C(=O)c1ccc(NC(=O)c2snc(-c3ccccc3)c2C2CC2)cc1Cl] |
| I-25 | 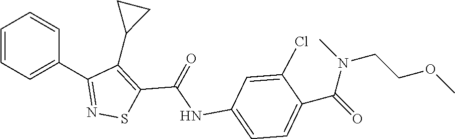 [SMILES: COCCN(C)C(=O)c1ccc(NC(=O)c2snc(-c3ccccc3)c2C2CC2)cc1Cl] |
| I-26 | 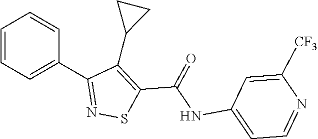 [SMILES: O=C(Nc1ccnc(C(F)(F)F)c1)c1snc(-c2ccccc2)c1C1CC1] |
| I-27 | 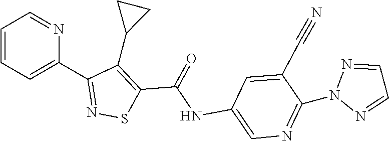 [SMILES: N#Cc1cc(NC(=O)c2snc(-c3ccccn3)c2C2CC2)cnc1-n1nccn1] |
| I-28 | 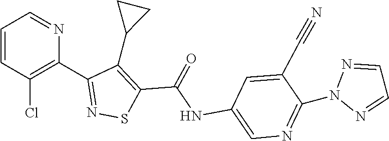 [SMILES: N#Cc1cc(NC(=O)c2snc(-c3ncccc3Cl)c2C2CC2)cnc1-n1nccn1] |
| I-29 | 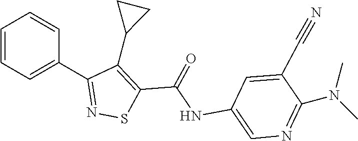 [SMILES: CN(C)c1ncc(NC(=O)c2snc(-c3ccccc3)c2C2CC2)cc1C#N] |
| I-30 | 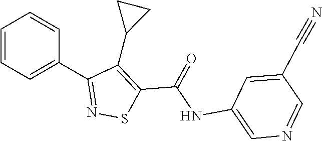 [SMILES: N#Cc1cncc(NC(=O)c2snc(-c3ccccc3)c2C2CC2)c1] |
| I-31 | 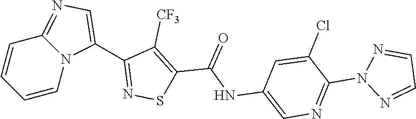 [SMILES: O=C(Nc1cnc(-n2nccn2)c(Cl)c1)c1snc(-c2cnc3ccccn23)c1C(F)(F)F] |
| I-32 | 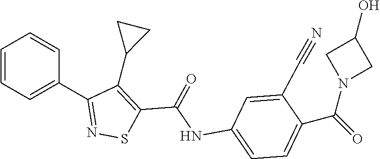 [SMILES: N#Cc1cc(NC(=O)c2snc(-c3ccccc3)c2C2CC2)ccc1C(=O)N1CC(O)C1] |
| I-33 | 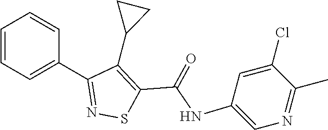 [SMILES: Cc1ncc(NC(=O)c2snc(-c3ccccc3)c2C2CC2)cc1Cl] |
| I-34 | 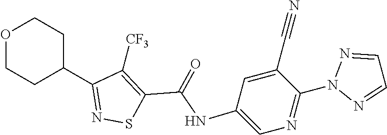 [SMILES: N#Cc1cc(NC(=O)c2snc(C3CCOCC3)c2C(F)(F)F)cnc1-n1nccn1] |
| I-35 | 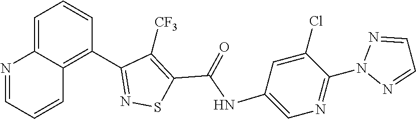 [SMILES: O=C(Nc1cnc(-n2nccn2)c(Cl)c1)c1snc(-c2cccc3ncccc23)c1C(F)(F)F] |
| I-36 | 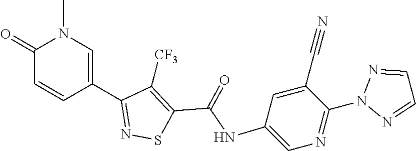 [SMILES: Cn1cc(-c2nsc(C(=O)Nc3cnc(-n4nccn4)c(C#N)c3)c2C(F)(F)F)ccc1=O] |
| I-37 | 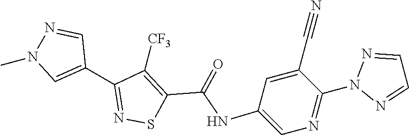 [SMILES: Cn1cc(-c2nsc(C(=O)Nc3cnc(-n4nccn4)c(C#N)c3)c2C(F)(F)F)cn1] |
| I-38 | 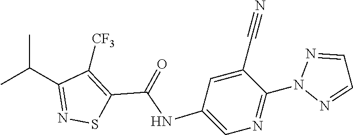 [SMILES: CC(C)c1nsc(C(=O)Nc2cnc(-n3nccn3)c(C#N)c2)c1C(F)(F)F] |
| I-39 | 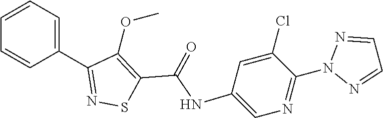 [SMILES: COc1c(-c2ccccc2)nsc1C(=O)Nc1cnc(-n2nccn2)c(Cl)c1] |
| I-40 | 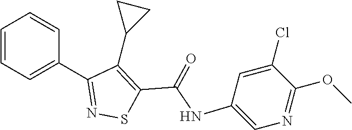 [SMILES: COc1ncc(NC(=O)c2snc(-c3ccccc3)c2C2CC2)cc1Cl] |
| I-41 | 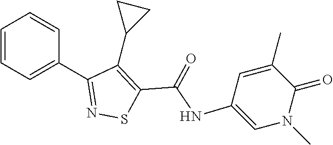 [SMILES: Cc1cc(NC(=O)c2snc(-c3ccccc3)c2C2CC2)cn(C)c1=O] |
| I-42 | 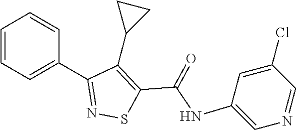 [SMILES: O=C(Nc1cncc(Cl)c1)c1snc(-c2ccccc2)c1C1CC1] |
| I-43 | 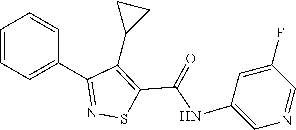 [SMILES: O=C(Nc1cncc(F)c1)c1snc(-c2ccccc2)c1C1CC1] |
| I-44 | 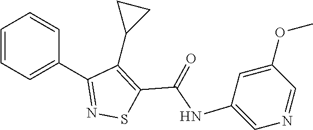 [SMILES: COc1cncc(NC(=O)c2snc(-c3ccccc3)c2C2CC2)c1] |
| I-45 | 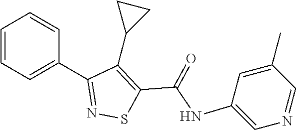 [SMILES: Cc1cncc(NC(=O)c2snc(-c3ccccc3)c2C2CC2)c1] |
| I-46 | 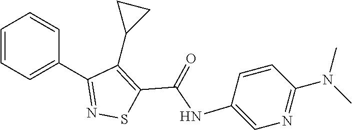 [SMILES: CN(C)c1ccc(NC(=O)c2snc(-c3ccccc3)c2C2CC2)cn1] |
| I-47 | 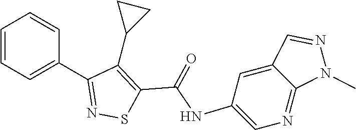 [SMILES: Cn1ncc2cc(NC(=O)c3snc(-c4ccccc4)c3C3CC3)cnc21] |
| I-48 | 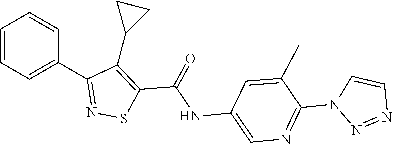 [SMILES: Cc1cc(NC(=O)c2snc(-c3ccccc3)c2C2CC2)cnc1-n1ccnn1] |
| I-49 | 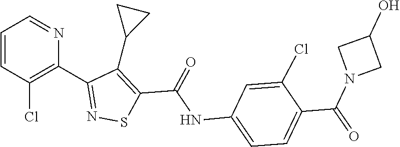 [SMILES: O=C(Nc1ccc(C(=O)N2CC(O)C2)c(Cl)c1)c1snc(-c2ncccc2Cl)c1C1CC1] |
| I-50 | 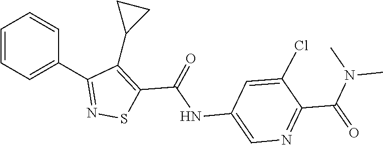 [SMILES: CN(C)C(=O)c1ncc(NC(=O)c2snc(-c3ccccc3)c2C2CC2)cc1Cl] |
| I-51 | 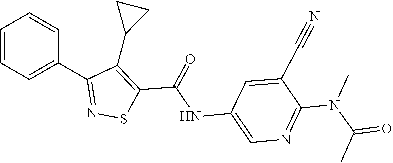 [SMILES: CC(=O)N(C)c1ncc(NC(=O)c2snc(-c3ccccc3)c2C2CC2)cc1C#N] |
| I-52 | 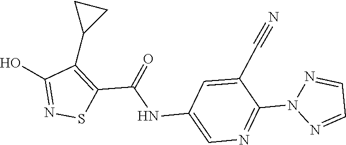 [SMILES: N#Cc1cc(NC(=O)c2snc(O)c2C2CC2)cnc1-n1nccn1] |
| I-53 | 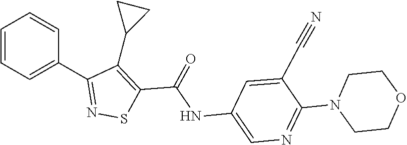 [SMILES: N#Cc1cc(NC(=O)c2snc(-c3ccccc3)c2C2CC2)cnc1N1CCOCC1] |
| I-54 | 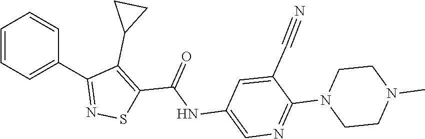 [SMILES: CN1CCN(c2ncc(NC(=O)c3snc(-c4ccccc4)c3C3CC3)cc2C#N)CC1] |
| I-55 | 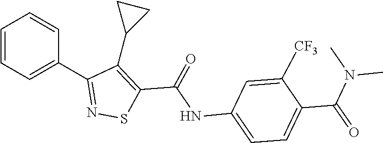 [SMILES: CN(C)C(=O)c1ccc(NC(=O)c2snc(-c3ccccc3)c2C2CC2)cc1C(F)(F)F] |
| I-56 | 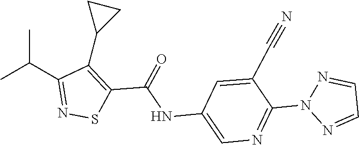 [SMILES: CC(C)c1nsc(C(=O)Nc2cnc(-n3nccn3)c(C#N)c2)c1C1CC1] |
| I-57 | 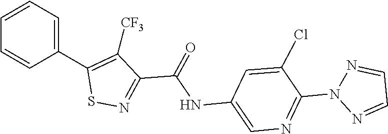 [SMILES: O=C(Nc1cnc(-n2nccn2)c(Cl)c1)c1nsc(-c2ccccc2)c1C(F)(F)F] |
| I-58 | 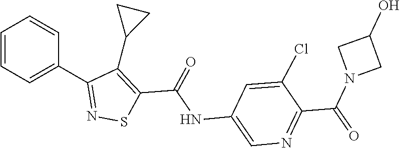 [SMILES: O=C(Nc1cnc(C(=O)N2CC(O)C2)c(Cl)c1)c1snc(-c2ccccc2)c1C1CC1] |
| I-59 | 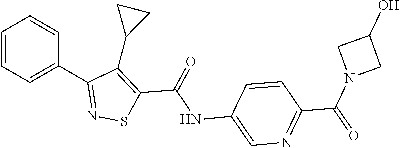 [SMILES: O=C(Nc1ccc(C(=O)N2CC(O)C2)nc1)c1snc(-c2ccccc2)c1C1CC1] |
| I-60 | 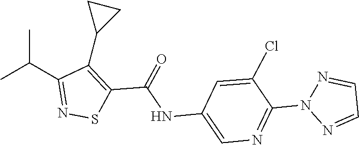 [SMILES: CC(C)c1nsc(C(=O)Nc2cnc(-n3nccn3)c(Cl)c2)c1C1CC1] |
| I-61 | 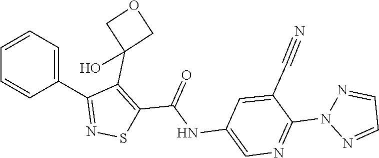 [SMILES: N#Cc1cc(NC(=O)c2snc(-c3ccccc3)c2C2(O)COC2)cnc1-n1nccn1] |
| I-62 | 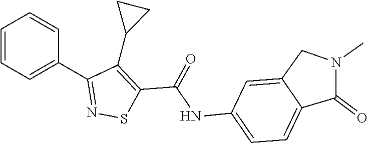 [SMILES: CN1Cc2cc(NC(=O)c3snc(-c4ccccc4)c3C3CC3)ccc2C1=O] |
| I-63 | 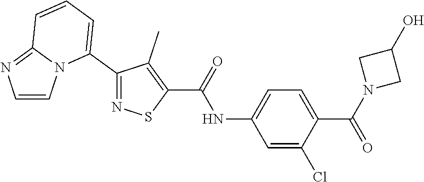 [SMILES: Cc1c(-c2cccc3nccn23)nsc1C(=O)Nc1ccc(C(=O)N2CC(O)C2)c(Cl)c1] |
| I-64 | 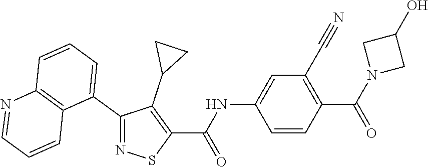 [SMILES: N#Cc1cc(NC(=O)c2snc(-c3cccc4ncccc34)c2C2CC2)ccc1C(=O)N1CC(O)C1] |
| I-65 | 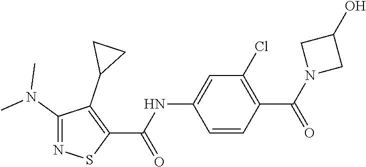 [SMILES: CN(C)c1nsc(C(=O)Nc2ccc(C(=O)N3CC(O)C3)c(Cl)c2)c1C1CC1] |
| I-66 | 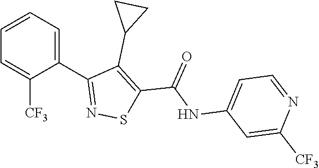 [SMILES: O=C(Nc1ccnc(C(F)(F)F)c1)c1snc(-c2ccccc2C(F)(F)F)c1C1CC1] |
| I-67 | 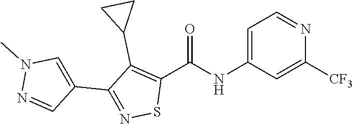 [SMILES: Cn1cc(-c2nsc(C(=O)Nc3ccnc(C(F)(F)F)c3)c2C2CC2)cn1] |
| I-68 | 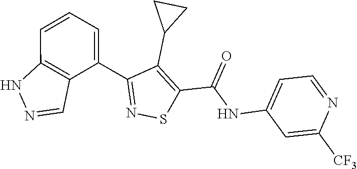 [SMILES: O=C(Nc1ccnc(C(F)(F)F)c1)c1snc(-c2cccc3[nH]ncc23)c1C1CC1] |
| I-69 | 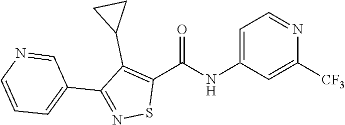 [SMILES: O=C(Nc1ccnc(C(F)(F)F)c1)c1snc(-c2cccnc2)c1C1CC1] |
| I-70 | 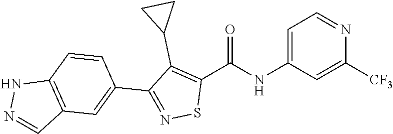 [SMILES: O=C(Nc1ccnc(C(F)(F)F)c1)c1snc(-c2ccc3[nH]ncc3c2)c1C1CC1] |
| I-71 | 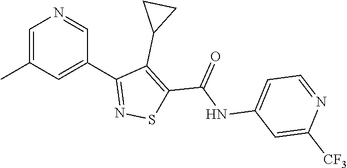 [SMILES: Cc1cncc(-c2nsc(C(=O)Nc3ccnc(C(F)(F)F)c3)c2C2CC2)c1] |
| I-72 | 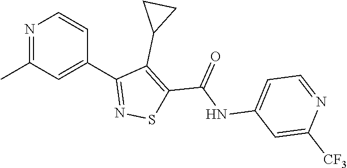 [SMILES: Cc1cc(-c2nsc(C(=O)Nc3ccnc(C(F)(F)F)c3)c2C2CC2)ccn1] |
| I-73 | 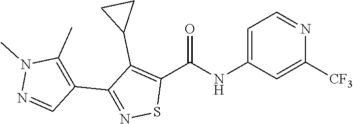 [SMILES: Cc1c(-c2nsc(C(=O)Nc3ccnc(C(F)(F)F)c3)c2C2CC2)cnn1C] |
| I-74 | 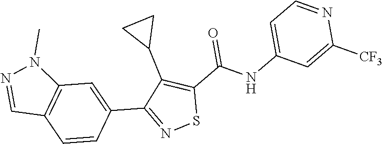 [SMILES: Cn1ncc2ccc(-c3nsc(C(=O)Nc4ccnc(C(F)(F)F)c4)c3C3CC3)cc21] |
| I-75 | 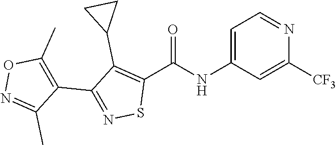 [SMILES: Cc1noc(C)c1-c1nsc(C(=O)Nc2ccnc(C(F)(F)F)c2)c1C1CC1] |
| I-76 | 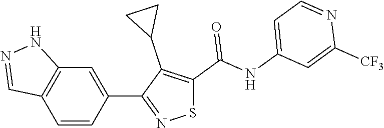 [SMILES: O=C(Nc1ccnc(C(F)(F)F)c1)c1snc(-c2ccc3cn[nH]c3c2)c1C1CC1] |
| I-77 | 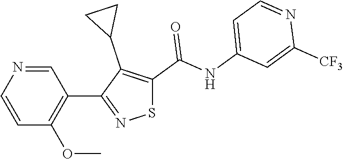 [SMILES: COc1ccncc1-c1nsc(C(=O)Nc2ccnc(C(F)(F)F)c2)c1C1CC1] |
| I-78 | 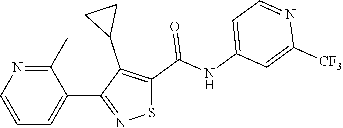 [SMILES: Cc1ncccc1-c1nsc(C(=O)Nc2ccnc(C(F)(F)F)c2)c1C1CC1] |
| I-79 | 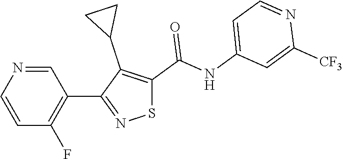 [SMILES: O=C(Nc1ccnc(C(F)(F)F)c1)c1snc(-c2cnccc2F)c1C1CC1] |
| I-80 | 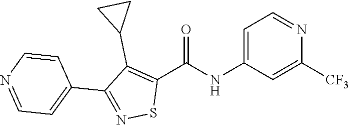 [SMILES: O=C(Nc1ccnc(C(F)(F)F)c1)c1snc(-c2ccncc2)c1C1CC1] |
| I-81 | 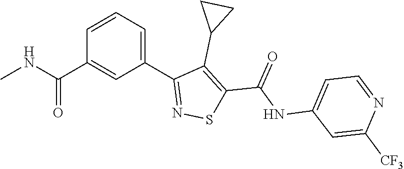 [SMILES: CNC(=O)c1cccc(-c2nsc(C(=O)Nc3ccnc(C(F)(F)F)c3)c2C2CC2)c1] |
| I-82 | 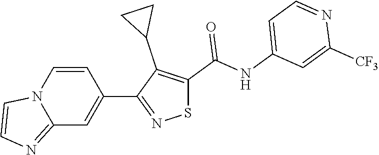 [SMILES: O=C(Nc1ccnc(C(F)(F)F)c1)c1snc(-c2ccn3ccnc3c2)c1C1CC1] |
| I-83 | 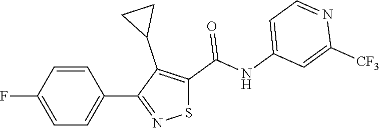 [SMILES: O=C(Nc1ccnc(C(F)(F)F)c1)c1snc(-c2ccc(F)cc2)c1C1CC1] |
| I-84 | 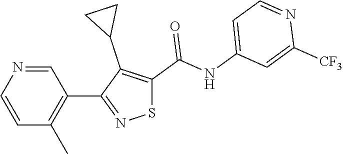 [SMILES: Cc1ccncc1-c1nsc(C(=O)Nc2ccnc(C(F)(F)F)c2)c1C1CC1] |
| I-85 | 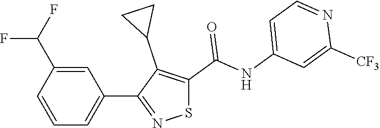 [SMILES: O=C(Nc1ccnc(C(F)(F)F)c1)c1snc(-c2cccc(C(F)F)c2)c1C1CC1] |
| I-86 | 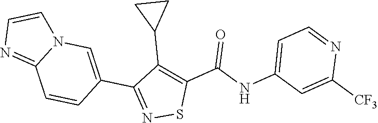 [SMILES: O=C(Nc1ccnc(C(F)(F)F)c1)c1snc(-c2ccc3nccn3c2)c1C1CC1] |
| I-87 | 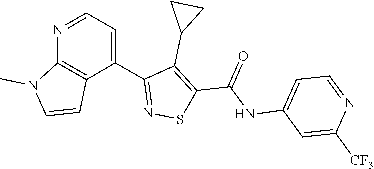 [SMILES: Cn1ccc2c(-c3nsc(C(=O)Nc4ccnc(C(F)(F)F)c4)c3C3CC3)ccnc21] |
| I-88 | 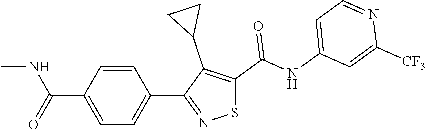 [SMILES: CNC(=O)c1ccc(-c2nsc(C(=O)Nc3ccnc(C(F)(F)F)c3)c2C2CC2)cc1] |
| I-89 | 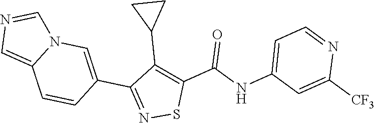 [SMILES: O=C(Nc1ccnc(C(F)(F)F)c1)c1snc(-c2ccc3cncn3c2)c1C1CC1] |
| I-90 | 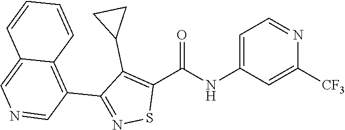 [SMILES: O=C(Nc1ccnc(C(F)(F)F)c1)c1snc(-c2cncc3ccccc23)c1C1CC1] |
| I-91 | 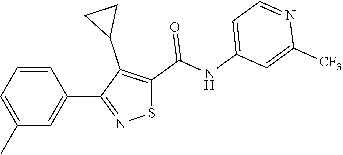 [SMILES: Cc1cccc(-c2nsc(C(=O)Nc3ccnc(C(F)(F)F)c3)c2C2CC2)c1] |
| I-92 | 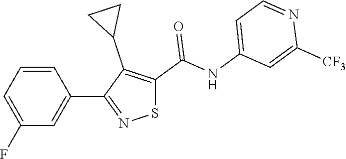 [SMILES: O=C(Nc1ccnc(C(F)(F)F)c1)c1snc(-c2cccc(F)c2)c1C1CC1] |
| I-93 | 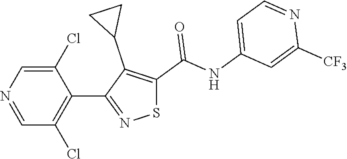 [SMILES: O=C(Nc1ccnc(C(F)(F)F)c1)c1snc(-c2c(Cl)cncc2Cl)c1C1CC1] |
| I-94 | 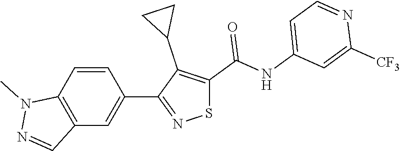 [SMILES: Cn1ncc2cc(-c3nsc(C(=O)Nc4ccnc(C(F)(F)F)c4)c3C3CC3)ccc21] |
| I-95 | 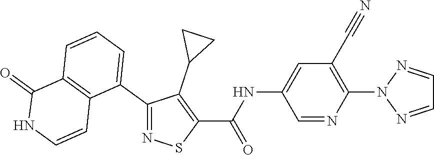 [SMILES: N#Cc1cc(NC(=O)c2snc(-c3cccc4c(=O)[nH]ccc34)c2C2CC2)cnc1-n1nccn1] |
| I-96 | 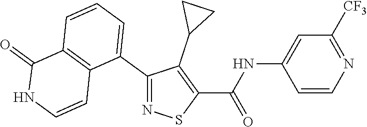 [SMILES: O=C(Nc1ccnc(C(F)(F)F)c1)c1snc(-c2cccc3c(=O)[nH]ccc23)c1C1CC1] |
| I-97 | 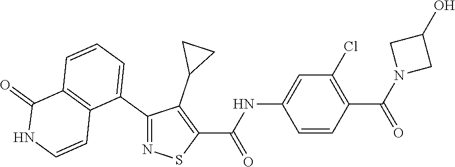 [SMILES: O=C(Nc1ccc(C(=O)N2CC(O)C2)c(Cl)c1)c1snc(-c2cccc3c(=O)[nH]ccc23)c1C1CC1] |
| I-98 | 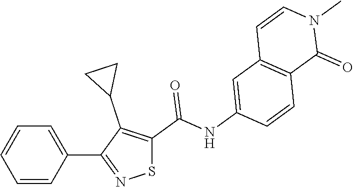 [SMILES: Cn1ccc2cc(NC(=O)c3snc(-c4ccccc4)c3C3CC3)ccc2c1=O] |
| I-99 | 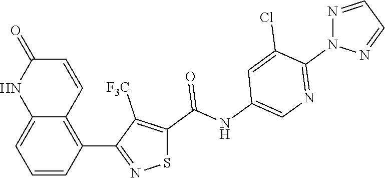 [SMILES: O=C(Nc1cnc(-n2nccn2)c(Cl)c1)c1snc(-c2cccc3[nH]c(=O)ccc23)c1C(F)(F)F] |
| I-100 | 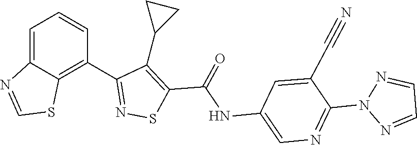 [SMILES: N#Cc1cc(NC(=O)c2snc(-c3cccc4ncsc34)c2C2CC2)cnc1-n1nccn1] |
| I-101 | 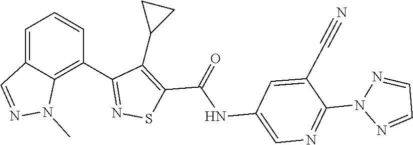 [SMILES: Cn1ncc2cccc(-c3nsc(C(=O)Nc4cnc(-n5nccn5)c(C#N)c4)c3C3CC3)c21] |
| I-102 | 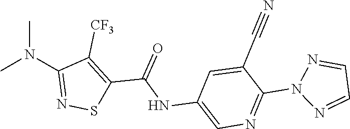 [SMILES: CN(C)c1nsc(C(=O)Nc2cnc(-n3nccn3)c(C#N)c2)c1C(F)(F)F] |
| I-103 | 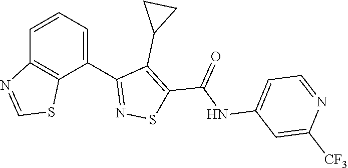 [SMILES: O=C(Nc1ccnc(C(F)(F)F)c1)c1snc(-c2cccc3ncsc23)c1C1CC1] |
| I-104 | 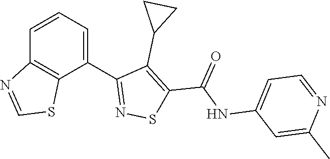 [SMILES: Cc1cc(NC(=O)c2snc(-c3cccc4ncsc34)c2C2CC2)ccn1] |
| I-105 | 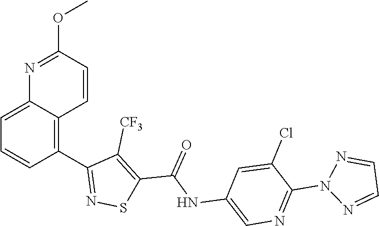 [SMILES: COc1ccc2c(-c3nsc(C(=O)Nc4cnc(-n5nccn5)c(Cl)c4)c3C(F)(F)F)cccc2n1] |
| I-106 | 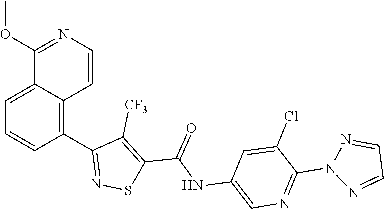 [SMILES: COc1nccc2c(-c3nsc(C(=O)Nc4cnc(-n5nccn5)c(Cl)c4)c3C(F)(F)F)cccc12] |
| I-107 | 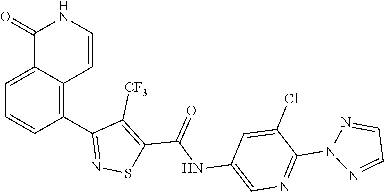 [SMILES: O=C(Nc1cnc(-n2nccn2)c(Cl)c1)c1snc(-c2cccc3c(=O)[nH]ccc23)c1C(F)(F)F] |
| I-108 | 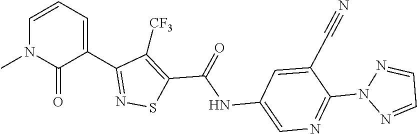 [SMILES: Cn1cccc(-c2nsc(C(=O)Nc3cnc(-n4nccn4)c(C#N)c3)c2C(F)(F)F)c1=O] |
| I-109 | 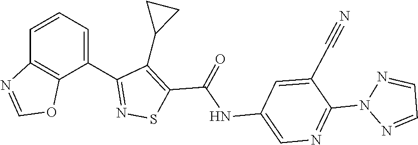 [SMILES: N#Cc1cc(NC(=O)c2snc(-c3cccc4ncoc34)c2C2CC2)cnc1-n1nccn1] |
| I-110 | 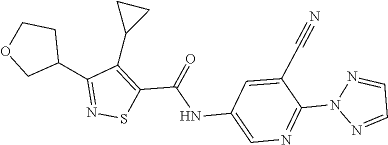 [SMILES: N#Cc1cc(NC(=O)c2snc(C3CCOC3)c2C2CC2)cnc1-n1nccn1] |
| I-111 | 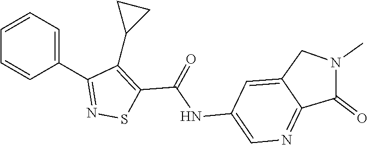 [SMILES: CN1Cc2cc(NC(=O)c3snc(-c4ccccc4)c3C3CC3)cnc2C1=O] |
| I-112 | 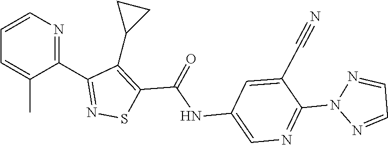 [SMILES: Cc1cccnc1-c1nsc(C(=O)Nc2cnc(-n3nccn3)c(C#N)c2)c1C1CC1] |
| I-113 | 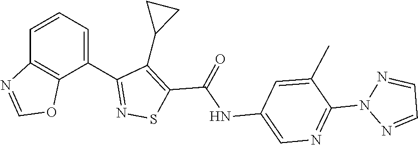 [SMILES: Cc1cc(NC(=O)c2snc(-c3cccc4ncoc34)c2C2CC2)cnc1-n1nccn1] |
| I-114 | 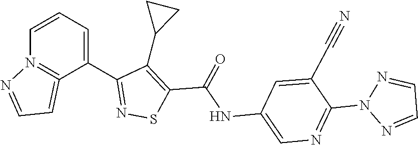 |
| I-115 | 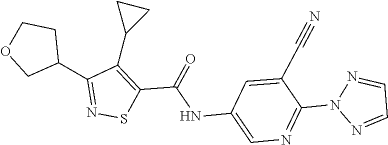 [SMILES: N#Cc1cc(NC(=O)c2snc(C3CCOC3)c2C2CC2)cnc1-n1nccn1] |
| I-116 | 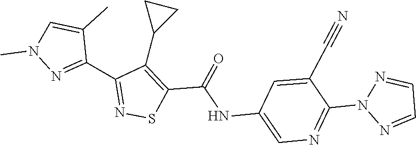 [SMILES: Cc1cn(C)nc1-c1nsc(C(=O)Nc2cnc(-n3nccn3)c(C#N)c2)c1C1CC1] |
| I-117 | 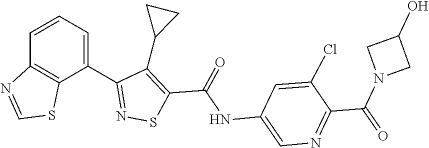 [SMILES: O=C(Nc1cnc(C(=O)N2CC(O)C2)c(Cl)c1)c1snc(-c2cccc3ncsc23)c1C1CC1] |
| I-118 | 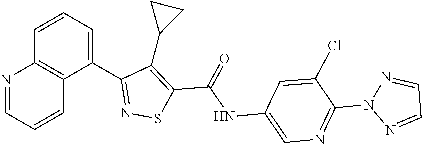 [SMILES: O=C(Nc1cnc(-n2nccn2)c(Cl)c1)c1snc(-c2cccc3ncccc23)c1C1CC1] |
| I-119 | 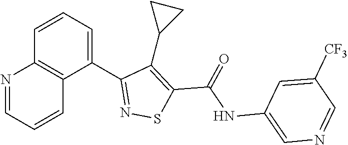 [SMILES: O=C(Nc1cncc(C(F)(F)F)c1)c1snc(-c2cccc3ncccc23)c1C1CC1] |
| I-120 | 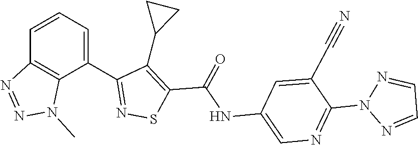 [SMILES: Cn1nnc2cccc(-c3nsc(C(=O)Nc4cnc(-n5nccn5)c(C#N)c4)c3C3CC3)c21] |
| I-121 | 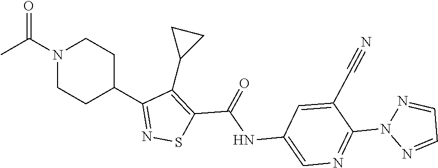 [SMILES: CC(=O)N1CCC(c2nsc(C(=O)Nc3cnc(-n4nccn4)c(C#N)c3)c2C2CC2)CC1] |
| I-122 | 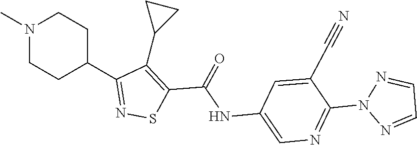 [SMILES: CN1CCC(c2nsc(C(=O)Nc3cnc(-n4nccn4)c(C#N)c3)c2C2CC2)CC1] |
| I-123 | 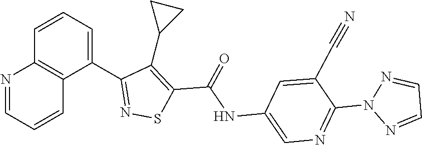 [SMILES: N#Cc1cc(NC(=O)c2snc(-c3cccc4ncccc34)c2C2CC2)cnc1-n1nccn1] |
| I-124 | 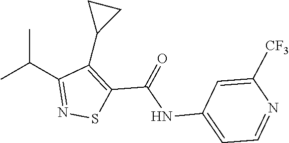 [SMILES: CC(C)c1nsc(C(=O)Nc2ccnc(C(F)(F)F)c2)c1C1CC1] |
| I-125 | 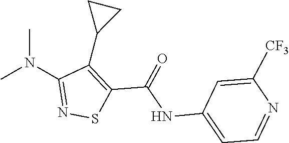 [SMILES: CN(C)c1nsc(C(=O)Nc2ccnc(C(F)(F)F)c2)c1C1CC1] |
| I-126 | 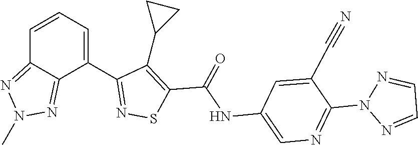 [SMILES: Cn1nc2cccc(-c3nsc(C(=O)Nc4cnc(-n5nccn5)c(C#N)c4)c3C3CC3)c2n1] |
| I-127 | 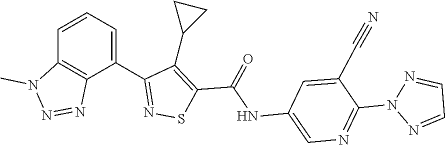 [SMILES: Cn1nnc2c(-c3nsc(C(=O)Nc4cnc(-n5nccn5)c(C#N)c4)c3C3CC3)cccc21] |
| I-128 | 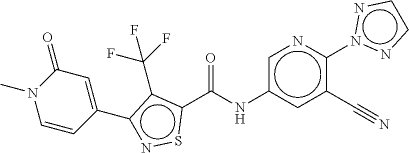 |
| I-129 | 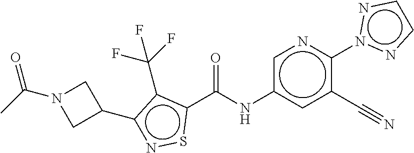 |
| I-130 | 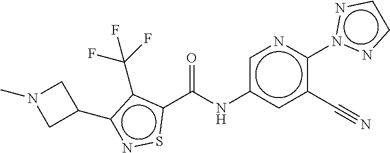 |
| I-131 | 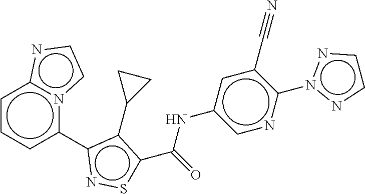 |
| I-132 | 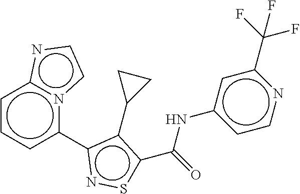 |
| I-133 | 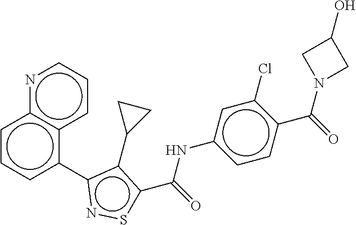 |
| I-134 | 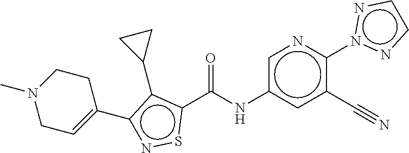 |
| I-135 | 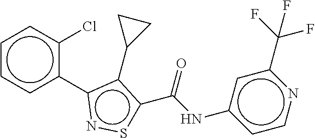 |
| I-136 | 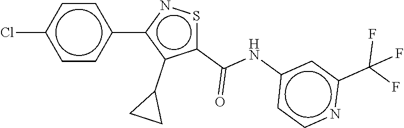 |
| I-137 | 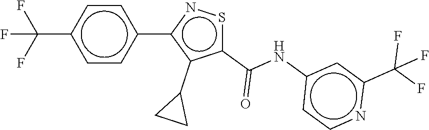 |
| I-138 | 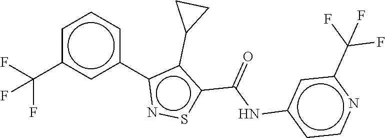 |
| I-139 | 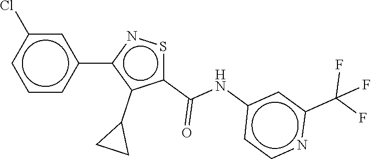 |
| I-140 | 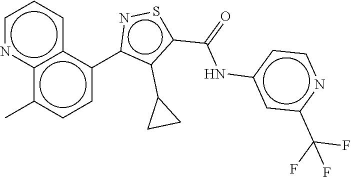 |
| I-141 | 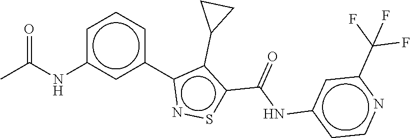 |
| I-142 | 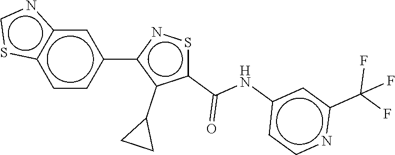 |
| I-143 | 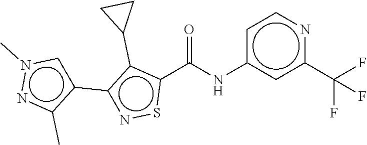 |
| I-144 | 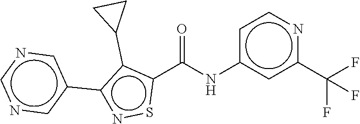 |
| I-145 | 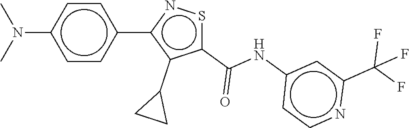 |
| I-146 | 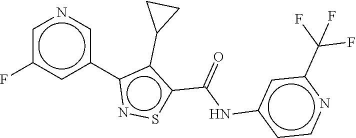 |
| I-147 | 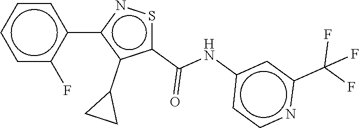 |
| I-148 | 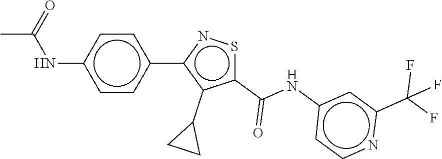 |
| I-149 | 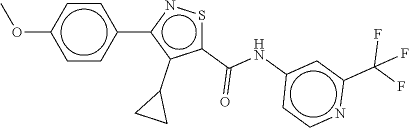 |
| I-150 | 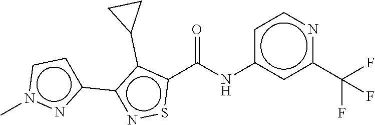 |
| I-151 | 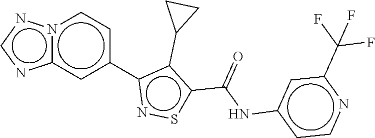 |
| I-152 | 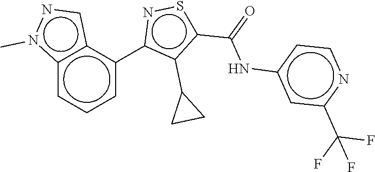 |
| I-153 | 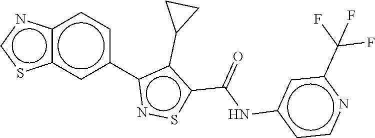 |
| I-154 | 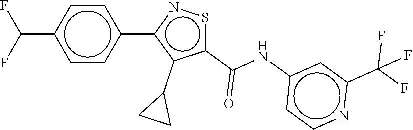 |
| I-155 | 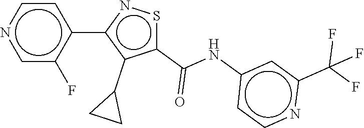 |
| I-156 | 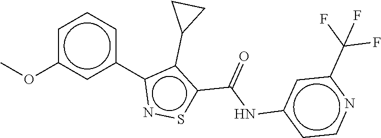 |
| I-157 | 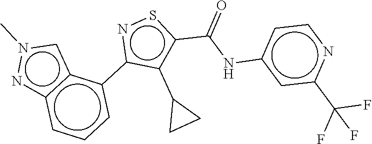 |
| I-158 | 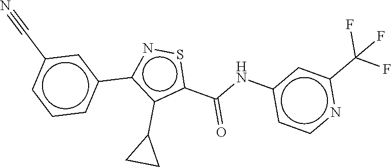 |
| I-159 | 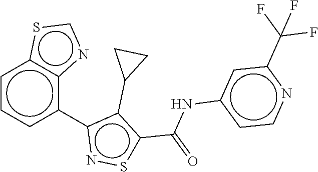 |
| I-160 | 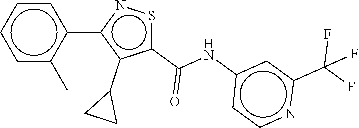 |
| I-161 | 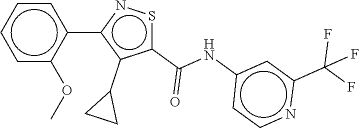 |
| I-162 | 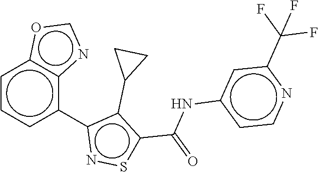 |
| I-163 | 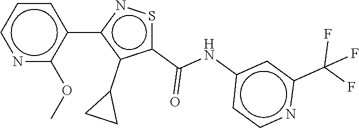 |
| I-164 | 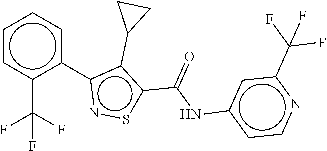 |
| I-165 | 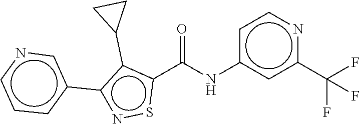 |
| I-166 | 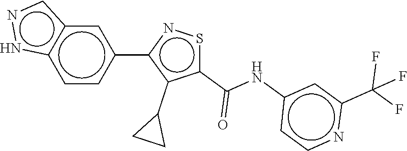 |
| I-167 | 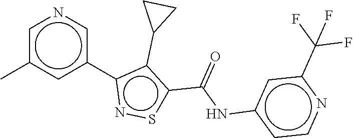 |
| I-168 | 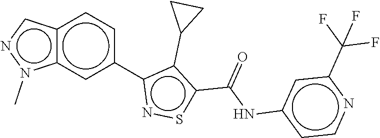 |
| I-169 | 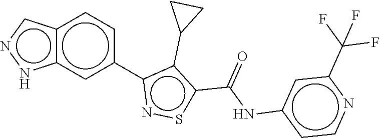 |
| I-170 | 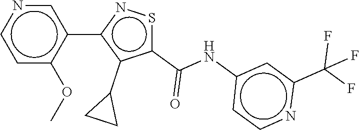 |
| I-171 | 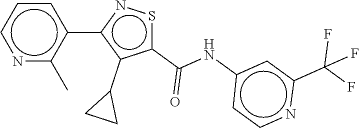 |
| I-172 | 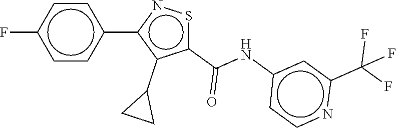 |
| I-173 | 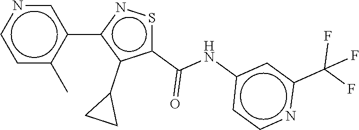 |
| I-174 | 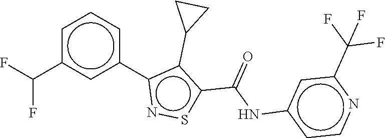 |
| I-175 | 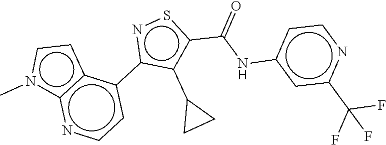 |
| I-176 | 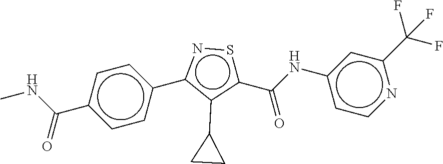 |
| I-177 | 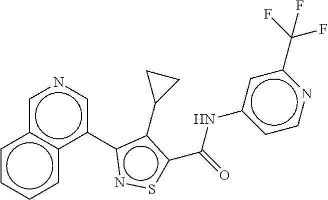 |
| I-178 | 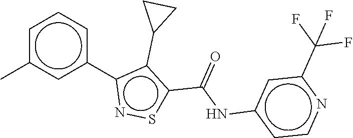 |
| I-179 | 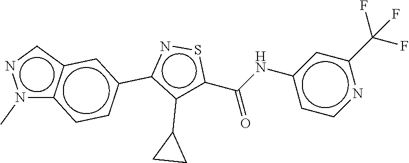 |
| I-180 | 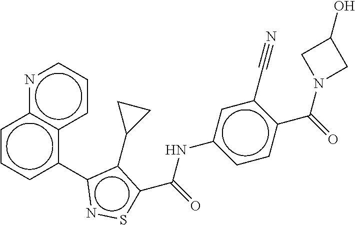 |
| I-181 | 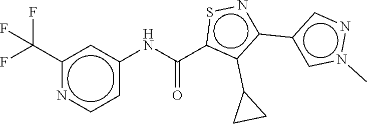 |
| I-182 | 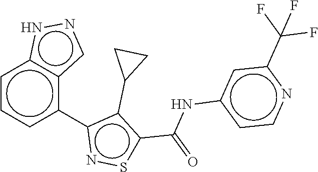 |
| I-183 | 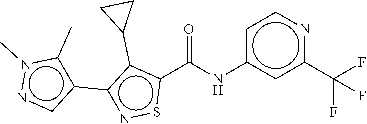 |
| I-184 | 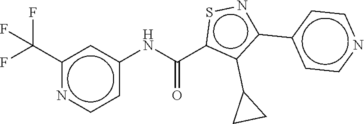 |
| I-185 | 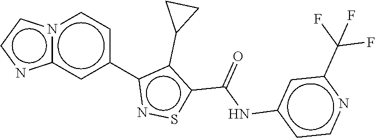 |
| I-186 | 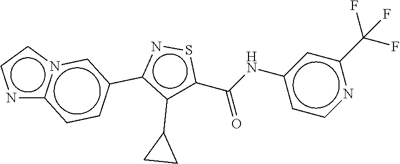 |
| I-187 | 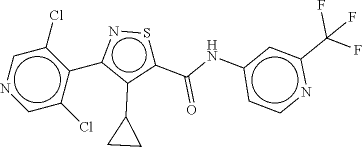 |
| I-188 | 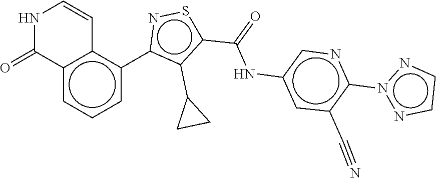 |
| I-189 | 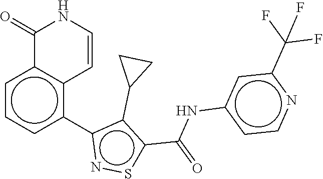 |
| I-190 | 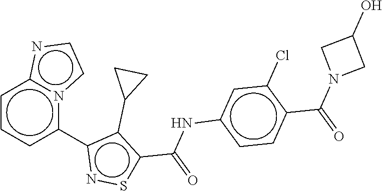 |
| I-191 | 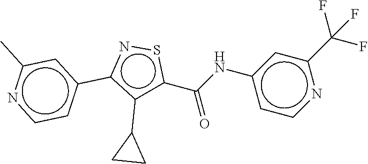 |
| I-192 | 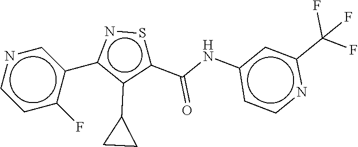 |
| I-193 | 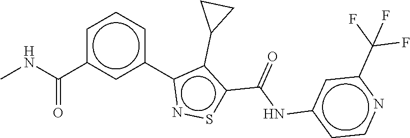 |
| I-194 | 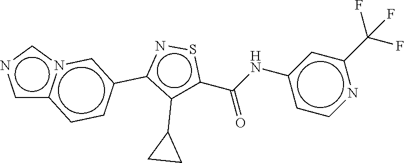 |
| I-195 | 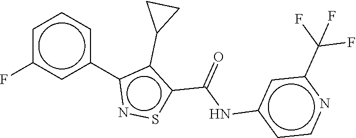 |
| I-196 | 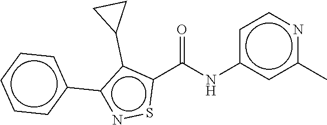 |
| I-197 | 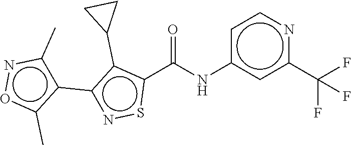 |
| I-198 | 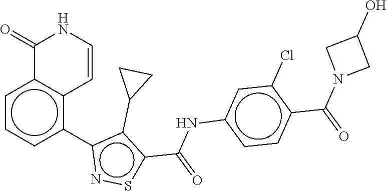 |
| I-199 | 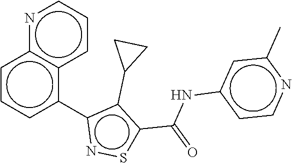 |
| I-200 | 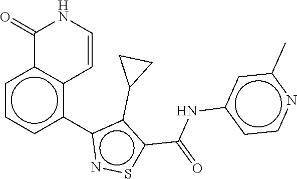 |
| I-201 | 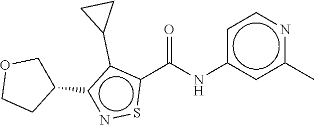 |
| I-202 | 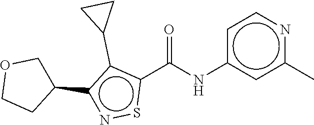 |
| I-203 | 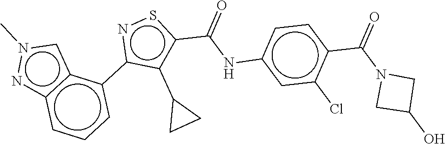 |
| I-204 | 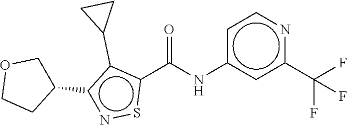 |
| I-205 | 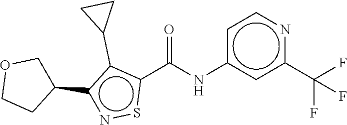 |
| I-206 | 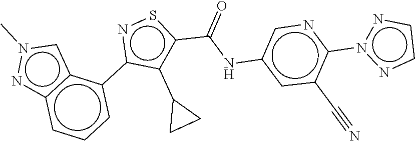 |
| I-207 | 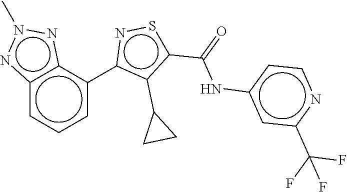 |
| I-208 | 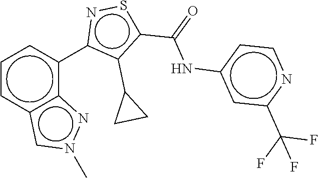 |
| I-209 | 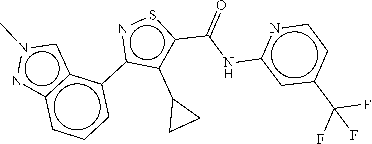 |
| I-210 | 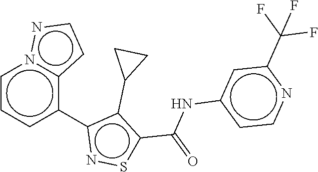 |
| I-211 | 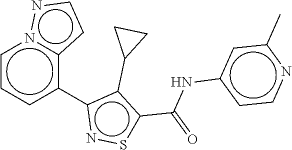 |
| I-212 | 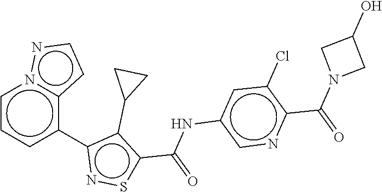 |

Methods for preparing compounds described herein are illustrated in the following synthetic scheme. The scheme is provided for the purpose of illustrating the invention, and is not intended to limit the scope or spirit of the invention. Starting materials shown in the scheme can be obtained from commercial sources or can be prepared based on procedures described in the literature.

In the scheme, it is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated (for example, use of protecting groups or alternative reactions). Protecting group chemistry and strategy is well known in the art, for example, as described in detail in Protecting Groups in Organic Synthesis, 3^{rd} Edition, T. W. Greene and P. G. M. Wuts, John Wiley & Sons, 1999 and Greene's Protective Groups in Organic Synthesis, 5th Ed., (Peter G. M. Wuts, John Wiley & Sons: 2014), the entire contents of both of which are hereby incorporated by reference.

The synthetic route illustrated in Scheme 1 is a general method for preparing isothiazolylcarboxamides C. Reaction of isothioazolylcarboxylic acid A with amine B under amide coupling conditions provides isothiazolylcarboxamide C.

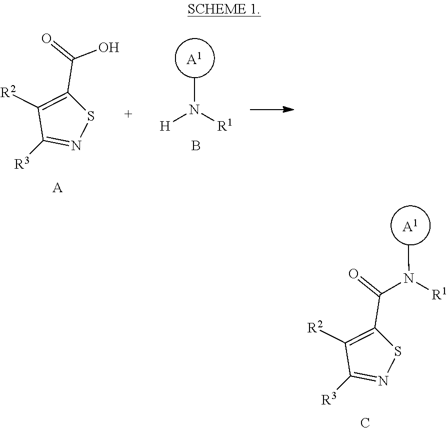

The modular synthetic route illustrated in Scheme 1 can be adjusted to provide additional isothiazolylcarboxamide compounds by conducting functional group transformations on the intermediate and final compounds. Such functional group transformations are well known in the art, as described in, for example, Comprehensive Organic Synthesis (B. M. Trost & I. Fleming, eds., 1991-1992); Organic Synthesis, ₃′d Ed (Michael B. Smith, Wavefunction, Inc., Irvine: 2010); Modern Methods of Organic Synthesis, 4^{th} Ed. (William Carruthers and Iain Coldham, Cambridge University Press, Cambridge: 2004); March's Advanced Organic Chemistry: Reactions, Mechanisms, and Structure, 8^{th} Ed., (Michael B. Smith, John Wiley & Sons, New York: 2020); and Comprehensive Organic Transformations: A Guide to Functional Group Preparations, 3rd Ed. (Richard C. Larock, ed., John Wiley & Sons, New York: 2018).

Certain ester intermediates can be prepared based on the general procedures described in WO 2011/059784. A representative synthesis of an ester intermediate is shown below in Scheme 2. Phenylacetonitrile 1 is reacted with isopentyl nitrite to give oxime 2. Oxime 2 is reacted with tosyl chloride to give (tosyloxy)benzimidoyl cyanide 3, which is reacted with methyl 2-mercaptoacetate to form isothiazole 4. Iodinated isothiazole 5 is formed from isothiazole 4 by reaction with iodine and isopentyl nitrite.

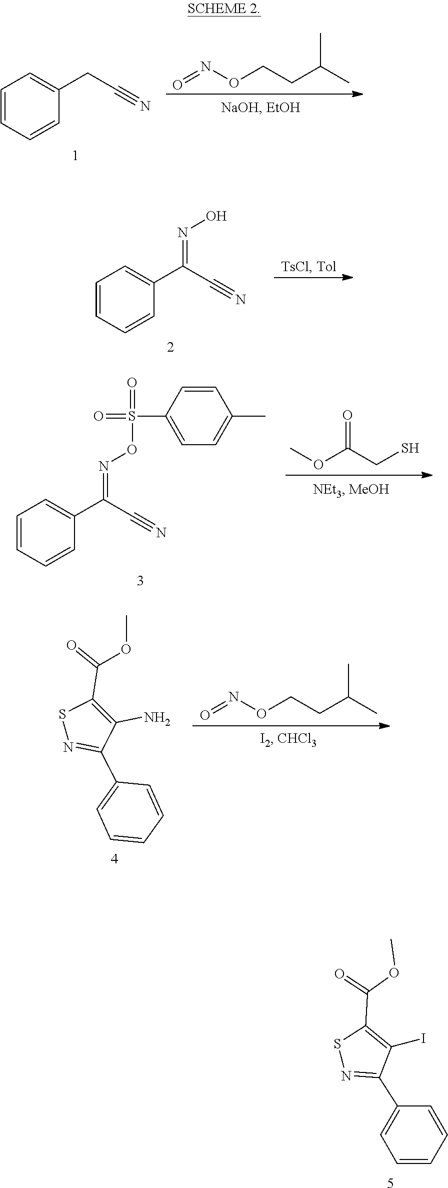

A representative synthesis of an isothiazolecarboxamide compound 8 is shown below in Scheme 3. Reaction of chloro-oxime 1 with KCN gives cyano oxime 2, which is reacted with tosyl chloride to give tosyloxy compound 3. Compound 3 is reacted with methyl 2-mercaptoacetate to form isothiazole 4, which is reacted with iodine and isopentyl nitrite to form iodinated isothiazole compound 5. Iodinated isothiazole compound 5 is then reacted with methyl 2,2-difluoro-2-(fluorosulfonyl)acetate in the presence of CuI to give trifluoromethyl-substituted isothiazole 6. Saponification of compound 6 gives carboxylic acid 7, which is then reacted with 5-chloro-6-(2H-1,2,3-triazol-2-yl)pyridin-3-amine and phosphorous oxychloride to give isothiazolecarboxamide compound 8.

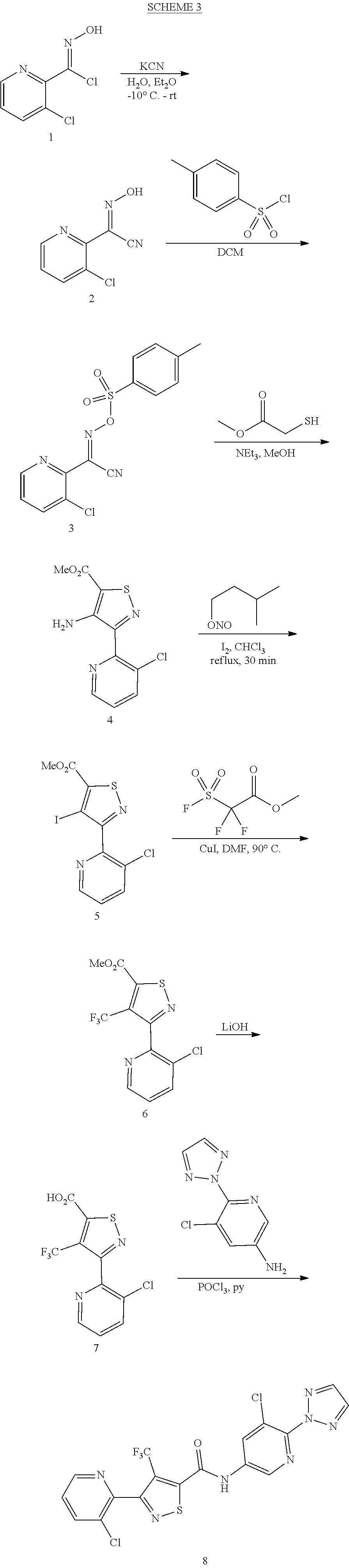

### II. Therapeutic Applications of Isothiazolylcarboxamide Compounds

Compounds described herein are useful for treating a disease or condition mediated by MALT1. Exemplary diseases or conditions mediated by MALT1 include proliferative disorders (e.g., cancer, neoplasia), inflammatory disorders (e.g., chronic inflammatory disorder, acute inflammatory disorder, auto-inflammatory disorder), autoimmune disorders, fibrotic disorders, metabolic disorders, cardiovascular disorders, cerebrovascular disorders, and myeloid cell-driven hyper-inflammatory responses in COVID-19 infections.

Accordingly, one aspect of the invention provides a method of treating a disease or condition mediated by MALT1 in a subject. The method comprises administering to a subject in need thereof a therapeutically effective amount of a compound described herein, such as a compound of Formula I or I-1, to treat the disease or condition. In certain embodiments, the compound is a compound of Formula I, Ia, Ib, Ic, Id, Ie, If, Ig, Ih, Ii, Ij, Ik, Il, Im, In, Io, Ip, Iq, Ir, Is, It, Tn, Iv, Iw, Ix, Iy, Iz, I-1, Ia-1, Tb-1, Ic-1, Id-1, Ie-1, If-1, Ig-1, Ih-1, Ii-1, Ij-1, Ik-1, Il-1, Im-1, In-1, Io-1, Ip-1, Iq-1, Ir-1, Is-1, It-1, Iu-1, Iv-1, Iw-1, or Ix-1, or other compounds in Section I, or defined by one of the embodiments described above. Further description of exemplary diseases or conditions mediated by MALT1 is provided herein below.

Another aspect of the invention provides a method of inhibiting the activity of MALT1. The method comprises contacting a MALT1 with an effective amount of a compound described herein, such as a compound of Formula I or T-1, to inhibit the activity of said MALT1. In certain embodiments, the compound is a compound of Formula I, Ia, Ib, Ic, Id, Ie, If, Ig, Ih, Ii, Ij, Ik, Il, Im, In, Io, Ip, Iq, Ir, Is, It, T, Iv, Iw, Ix, Iy, Iz, T-1, Ia-1, Ib-1, Ic-1, Id-1, Te-1, If-1, Ig-1, Ih-1, Ii-1, Ij-1, Ik-1, Il-1, Im-1, In-1, Io-1, Ip-1, Iq-1, Ir-1, Is-1, It-1, Iu-1, Iv-1, Iw-1, or Ix-1, or other compounds in Section I, or defined by one of the embodiments described above.

Another aspect of the invention provides for the use of a compound described herein (such as a compound of Formula I, Ia, Ib, Ic, Id, Ie, If, Ig, Ih, Ii, Ij, Ik, Il, Im, In, Io, Ip, Iq, Ir, Is, It, Iu, Iv, Iw, Ix, Iy, Iz, I-1, Ia-1, Ib-1, Ic-1, Id-1, Te-1, If-1, Ig-1, Ih-1, Ii-1, Ij-1, Ik-1, Il-1, Im-1, In-1, Io-1, Ip-1, Iq-1, Ir-1, Is-1, It-1, Iu-1, Iv-1, Iw-1, or Ix-1, or other compounds in Section I) in the manufacture of a medicament. In certain embodiments, the medicament is for treating a disease or condition described herein, such as an inflammatory disorder or an allergic disorder. In certain embodiments, the compound is a compound of Formula I, Ia, Ib, Ic, Id, Ie, If, Ig, Ih, Ii, Ij, Ik, Ii, Im, In, Io, Ip, Iq, Ir, Is, It, Iu, Iv, Iw, Ix, Iy, Iz, T-1, Ia-1, Ib-1, Ic-1, Id-1, Te-1, If-1, Ig-1, Ih-1, Ii-1, Ij-1, Ik-1, Il-1, Im-1, In-1, Io-1, Ip-1, Iq-1, Ir-1, Is-1, It-1, Iu-1, Iv-1, Iw-1, or Ix-1, or other compounds in Section I, or defined by one of the embodiments described above.

Another aspect of the invention provides for the use of a compound described herein (such as a compound of Formula I, Ia, Ib, Ic, Id, Ie, If, Ig, Ih, Ii, Ij, Ik, Il, Im, In, Io, Ip, Iq, Ir, Is, It, Iu, Iv, Iw, Ix, Iy, Iz, I-1, Ia-1, Ib-1, Ic-1, Id-1, Te-1, If-1, Ig-1, Ih-1, Ii-1, Ij-1, Ik-1, Il-1, Im-1, In-1, Io-1, Ip-1, Iq-1, Ir-1, Is-1, It-1, Iu-1, Iv-1, Iw-1, or Ix-1, or other compounds in Section I) for treating a disease or condition, such as a disease or condition described herein. In certain embodiments, the compound is a compound of Formula I, Ia, Ib, Ic, Id, Ie, If, Ig, Ih, Ii, Ij, Ik, Il, Im, In, Io, Ip, Iq, Ir, Is, It, Iu, Iv, Iw, Ix, Iy, Iz, I-1, Ia-1, Ib-1, Ic-1, Id-1, Ie-1, If-1, Ig-1, Ih-1, Ii-1, Ij-1, Ik-1, Il-1, Im-1, In-1, Io-1, Ip-1, Iq-1, Ir-1, Is-1, It-1, Iu-1, Iv-1, Iw-1, or Ix-1, or other compounds in Section I, or defined by one of the embodiments described above.

In certain embodiments, the subject is a human. In certain embodiments, the subject is an adult human. In certain embodiments, the subject is a pediatric human. In certain embodiments, the subject is a geriatric human.

### Exemplary Diseases or Conditions

Exemplary diseases or conditions mediated by MALT1 include proliferative disorders (e.g., cancer, neoplasia), inflammatory disorders (e.g., chronic inflammatory disorder, acute inflammatory disorder, auto-inflammatory disorder), autoimmune disorders, fibrotic disorders, metabolic disorders, cardiovascular disorders, cerebrovascular disorders, and myeloid cell-driven hyper-inflammatory responses in COVID-19 infections.

In certain embodiments, the disease or condition mediated by MALT1 is a proliferative disorder. In certain embodiments, the disease or condition mediated by MALT1 is inflammatory disorder. In certain embodiments, the disease or condition mediated by MALT1 is an autoimmune disorder. In certain embodiments, the disease or condition mediated by MALT1 is a fibrotic disorder. In certain embodiments, the disease or condition mediated by MALT1 is a metabolic disorder. In certain embodiments, the disease or condition mediated by MALT1 is a cardiovascular disorder. In certain embodiments, the disease or condition mediated by MALT1 is a cerebrovascular disorder. In certain embodiments, the disease or condition mediated by MALT1 is a myeloid cell-driven hyper-inflammatory response in a COVID-19 infection.

In certain embodiments, the disease or condition mediated by MALT1 is cancer.

In certain embodiments, the cancer is selected from is non-small cell lung cancer (NSCLC), small cell lung cancer, colorectal cancer, rectal cancer, and pancreatic cancer. In certain embodiments, the cancer is selected from non-small cell lung cancer (NSCLC), pancreatic cancer, and colorectal cancer. In certain embodiments, the cancer is selected from non-small cell lung cancer (NSCLC) and pancreatic cancer.

In certain embodiments, the cancer is a solid tumor. In certain embodiments, the cancer is a melanoma, carcinoma, or blastoma. In certain embodiments, the cancer is a melanoma. In certain embodiments, the cancer is a carcinoma. In certain embodiments, the cancer is an adenocarcinoma. In certain embodiments, the cancer is a blastoma.

In certain embodiments, the cancer is lung cancer, pancreatic cancer, colorectal cancer, breast cancer, cervical cancer, prostate cancer, gastric cancer, skin cancer, liver cancer, bile duct cancer, nervous system cancer, a lymphoma, or a leukemia. In certain embodiments, the cancer is lung cancer. In certain embodiments, the cancer is pancreatic cancer. In certain embodiments, the cancer is colorectal cancer. In certain embodiments, the cancer is breast cancer. In certain embodiments, the cancer is cervical cancer. In certain embodiments, the cancer is prostate cancer. In certain embodiments, the cancer is gastric cancer. In certain embodiments, the cancer is skin cancer. In certain embodiments, the cancer is liver cancer. In certain embodiments, the cancer is bile duct cancer. In certain embodiments, the cancer is nervous system cancer.

In certain embodiments, the cancer is breast adenocarcinoma, lung adenocarcinoma, pancreatic adenocarcinoma, cervical adenocarcinoma, colorectal adenocarcinoma, prostate adenocarcinoma, gastric adenocarcinoma, melanoma, lung squamous cell carcinoma, hepatocellular carcinoma, cholangiocarcinoma, glioblastoma, or neuroblastoma. In certain embodiments, the cancer is breast adenocarcinoma. In certain embodiments, the cancer is lung adenocarcinoma. In certain embodiments, the cancer is pancreatic adenocarcinoma. In certain embodiments, the cancer is cervical adenocarcinoma. In certain embodiments, the cancer is prostate adenocarcinoma. In certain embodiments, the cancer is gastric adenocarcinoma.

In certain embodiments, the cancer is melanoma.

In certain embodiments, the cancer is lung squamous cell carcinoma, hepatocellular carcinoma, or cholangiocarcinoma. In certain embodiments, the cancer is lung squamous cell carcinoma. In certain embodiments, the cancer is hepatocellular carcinoma. In certain embodiments, the cancer is cholangiocarcinoma.

In certain embodiments, the cancer is glioblastoma or neuroblastoma. In certain embodiments, the cancer is glioblastoma. In certain embodiments, the cancer is neuroblastoma.

In certain embodiments, the cancer is lung cancer, pancreatic cancer, or colorectal cancer. In certain embodiments, the cancer is non-small cell lung cancer, pancreatic cancer, or colorectal cancer. In certain embodiments, the cancer is lung cancer. In certain embodiments, the cancer is non-small cell lung cancer.

In certain embodiments, the cancer is a lymphoma or leukemia. In certain embodiments, the cancer is a B-cell lymphoma or chronic myelocytic leukemia.

In certain embodiments, the cancer is a leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, or a solid tumor such as a sarcoma or carcinoma (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, and retinoblastoma).

In certain embodiments, the cancer is MALT1 is Hodgkin's lymphoma, non-Hodgkin's lymphoma, Burkitt's lymphoma, diffuse large B-cell lymphoma (DLBCL), MALT lymphoma, germinal center B-cell-like diffuse large B-cell lymphoma (GCB-DLBCL), primary mediastinal B-cell lymphoma (PMBL), or activated B-cell-like diffuse large B-cell lymphoma (ABC-DLBCL).

In certain embodiments, the cancer is glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, or retinoblastoma.

In certain embodiments, the cancer is acoustic neuroma, astrocytoma (e.g. Grade I—Pilocytic Astrocytoma, Grade II—Low-grade Astrocytoma, Grade III—Anaplastic Astrocytoma, or Grade IV—Glioblastoma (GBM)), chordoma, CNS lymphoma, craniopharyngioma, brain stem glioma, ependymoma, mixed glioma, optic nerve glioma, subependymoma, medulloblastoma, meningioma, metastatic brain tumor, oligodendroglioma, pituitary tumors, primitive neuroectodermal (PNET) tumor, or schwannoma. In certain embodiments, the cancer is a type found more commonly in children than adults, such as brain stem glioma, craniopharyngioma, ependymoma, juvenile pilocytic astrocytoma (JPA), medulloblastoma, optic nerve glioma, pineal tumor, primitive neuroectodermal tumors (PNET), or rhabdoid tumor.

In certain embodiments, the cancer is mesothelioma, hepatobilliary (hepatic and billiary duct), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In certain embodiments, the cancer is hepatocellular carcinoma, ovarian cancer, ovarian epithelial cancer, fallopian tube cancer, papillary serous cystadenocarcinoma, uterine papillary serous carcinoma (UPSC), prostate cancer, testicular cancer, gallbladder cancer, hepatocholangiocarcinoma, soft tissue and bone synovial sarcoma, rhabdomyosarcoma, osteosarcoma, chondrosarcoma, Ewing sarcoma, anaplastic thyroid cancer, adrenocortical adenoma, pancreatic cancer, pancreatic ductal carcinoma, pancreatic adenocarcinoma, gastrointestinal/stomach (GIST) cancer, lymphoma, squamous cell carcinoma of the head and neck (SCCHN), salivary gland cancer, glioma, or brain cancer, neurofibromatosis-1 associated malignant peripheral nerve sheath tumors (MPNST), Waldenstrom's macroglobulinemia, or medulloblastoma.

In certain embodiments, the cancer is hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In certain embodiments, the cancer is selected from renal cell carcinoma, or kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC): prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; and medulloblastoma.

In certain embodiments, the cancer is renal cell carcinoma, hepatocellular carcinoma (HCC), hepatoblastoma, colorectal carcinoma, colorectal cancer, colon cancer, rectal cancer, anal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, chondrosarcoma, anaplastic thyroid cancer, adrenocortical carcinoma, pancreatic cancer, pancreatic ductal carcinoma, pancreatic adenocarcinoma, glioma, brain cancer, neurofibromatosis-1 associated malignant peripheral nerve sheath tumors (MPNST), Waldenstrom's macroglobulinemia, or medulloblastoma.

In certain embodiments, the cancer is hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In certain embodiments, the cancer is hepatocellular carcinoma (HCC). In certain embodiments, the cancer is hepatoblastoma. In certain embodiments, the cancer is colon cancer. In certain embodiments, the cancer is rectal cancer. In certain embodiments, the cancer is ovarian cancer, or ovarian carcinoma. In certain embodiments, the cancer is ovarian epithelial cancer. In certain embodiments, the cancer is fallopian tube cancer. In certain embodiments, the cancer is papillary serous cystadenocarcinoma. In certain embodiments, the cancer is uterine papillary serous carcinoma (UPSC). In certain embodiments, the cancer is hepatocholangiocarcinoma. In certain embodiments, the cancer is soft tissue and bone synovial sarcoma. In certain embodiments, the cancer is rhabdomyosarcoma. In certain embodiments, the cancer is osteosarcoma. In certain embodiments, the cancer is anaplastic thyroid cancer. In certain embodiments, the cancer is adrenocortical carcinoma. In certain embodiments, the cancer is pancreatic cancer, or pancreatic ductal carcinoma. In certain embodiments, the cancer is pancreatic adenocarcinoma. In certain embodiments, the cancer is glioma. In certain embodiments, the cancer is malignant peripheral nerve sheath tumors (MPNST). In certain embodiments, the cancer is neurofibromatosis-1 associated MPNST. In certain embodiments, the cancer is Waldenstrom's macroglobulinemia. In certain embodiments, the cancer is medulloblastoma.

In certain embodiments, the cancer is a lymphoma. In certain embodiments, the cancer is a leukemia. In certain embodiments, the cancer is Hodgkin's lymphoma. In certain embodiments, the cancer is non-Hodgkin's lymphoma. In certain embodiments, the cancer is Burkitt's lymphoma. In certain embodiments, the cancer is diffuse large B-cell lymphoma (DLBCL). In certain embodiments, the cancer is MALT lymphoma. In certain embodiments, the cancer is germinal center B-cell-like diffuse large B-cell lymphoma (GCB-DLBCL) or primary mediastinal B-cell lymphoma (PMBL). In certain embodiments, the cancer is activated B-cell-like diffuse large B-cell lymphoma (ABC-DLBCL). In certain embodiments, the cancer is a hematological cancer.

In certain embodiments, the proliferative disease is a cancer associated with or dependent on a MALT1 fusion protein (e.g., API2-MALTT). In certain embodiments, the proliferative disease is a cancer associated with dependence on B-cell lymphoma 10 (Bcl10). In certain embodiments, the proliferative disease is a cancer associated with dependence on caspase recruitment domain-containing protein (CARD1). In certain embodiments, the proliferative disease is a cancer associated with dependence on NF-κB. In certain embodiments, the cancer is a hematological malignancy.

Additional exemplary cancers include but are not limited to acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast, triple negative breast cancer (TNBC)); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); heavy chain disease (e.g., alpha chain disease, gamma chain disease, mu chain disease; hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; vulvar cancer (e.g., Paget's disease of the vulva); Burkitt lymphoma; primary intraocular lymphoma; classic Hodgkin lymphoma; biphenotypic acute leukemia; T cell lymphoma; nasal-type T cell lymphoma; enteropathy-type T-cell lymphoma, subcutaneous panniculitis-like T-cell lymphoma; blastic NK-cell lymphoma; T-cell prolymphocytic leukemia, and N-cell leukemia.

In certain embodiments, the cancer is a hematological malignancy. Exemplary hematological malignancies include but are not limited to leukemia, such as acute lymphoblastic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), chronic lymphocytic leukemia (CLL) (e.g., B-cell CLL, T-cell CLL)), acute non-lymphocytic leukemia (ANLL), acute promyelocytic leukemia (APL), and acute myelomonocytic leukemia (AMMoL); lymphoma, such as Hodgkin lymphoma (HL) (e.g., B-cell HL, T-cell HL) and non-Hodgkin lymphoma (NHL) (e.g., B-cell NHL, such as diffuse large cell lymphoma (DLCL) (e.g., diffuse large B-cell lymphoma (DLBCL, e.g., activated B-cell (ABC) DLBCL (ABC-DLBCL))), follicular lymphoma, chronic lymphocytic leukemia/small lymphocytic lymphoma (CLL/SLL), mantle cell lymphoma (MCL), marginal zone B-cell lymphoma (e.g., mucosa-associated lymphoid tissue (MALT) lymphoma, nodal marginal zone B-cell lymphoma, splenic marginal zone B-cell lymphoma), primary mediastinal B-cell lymphoma, Burkitt's lymphoma, Waldenstrom's macroglobulinemia (WM, lymphoplasmacytic lymphoma), hairy cell leukemia (HCL), immunoblastic large cell lymphoma, precursor B-lymphoblastic lymphoma, central nervous system (CNS) lymphoma (e.g., primary CNS lymphoma and secondary CNS lymphoma); and T-cell NHL, such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); lymphoma of an immune privileged site (e.g., cerebral lymphoma, ocular lymphoma, lymphoma of the placenta, lymphoma of the fetus, testicular lymphoma); a mixture of one or more leukemia/lymphoma as described above; myelodysplasia; multiple myeloma (MM); heavy chain disease (e.g., alpha chain disease, gamma chain disease, mu chain disease), polycythemia vera, Wilm's tumor, and Ewing's sarcoma.

In certain embodiments, said disease or condition mediated by MALT1 is a multiple myeloma. In certain embodiments, said disease or condition mediated by MALT1 is a leukemia (e.g., acute lymphocytic leukemia, acute and chronic myelogenous leukemia, chronic lymphocytic leukemia, acute lymphoblastic leukemia, chronic myelomonocytic leukemia, or promyelocytic leukemia).

In certain embodiments, said disease or condition mediated by MALT1 is a lymphoma (e.g., B-cell lymphoma, T-cell lymphoma, mantle cell lymphoma, diffuse large B cell lymphoma, hairy cell lymphoma, Burkitt's lymphoma, mast cell tumors, Hodgkin's disease or non-Hodgkin's disease). In certain embodiments, said disease or condition mediated by MALT1 is myelodysplastic syndrome. In certain embodiments, said disease or condition mediated by MALT1 is fibrosarcoma. In certain embodiments, said disease or condition mediated by MALT1 is rhabdomyosarcoma. In certain embodiments, said disease or condition mediated by MALT1 is astrocytoma. In certain embodiments, said disease or condition mediated by MALT1 is neuroblastoma. In certain embodiments, said disease or condition mediated by MALT1 is glioma and schwannomas. In certain embodiments, said disease or condition mediated by MALT1 is melanoma. In certain embodiments, said disease or condition mediated by MALT1 is seminoma. In certain embodiments, said disease or condition mediated by MALT1 is teratocarcinoma. In certain embodiments, said disease or condition mediated by MALT1 is osteosarcoma. In certain embodiments, said disease or condition mediated by MALT1 is xenoderma pigmentosum. In certain embodiments, said disease or condition mediated by MALT1 is keratoctanthoma. In certain embodiments, said disease or condition mediated by MALT1 is thyroid follicular cancer. In certain embodiments, said disease or condition mediated by MALT1 is Kaposi's sarcoma. In certain embodiments, said disease or condition mediated by MALT1 is melanoma. In certain embodiments, said disease or condition mediated by MALT1 is teratoma. In certain embodiments, said disease or condition mediated by MALT1 is rhabdomyosarcoma. In certain embodiments, said disease or condition mediated by MALT1 is a metastatic and bone disorder. In certain embodiments, said disease or condition mediated by MALT1 is cancer of the bone. In certain embodiments, said disease or condition mediated by MALT1 is cancer of the mouth/pharynx. In certain embodiments, said disease or condition mediated by MALT1 is cancer of the esophagus. In certain embodiments, said disease or condition mediated by MALT1 is cancer of the larynx. In certain embodiments, said disease or condition mediated by MALT1 is cancer of the stomach. In certain embodiments, said disease or condition mediated by MALT1 is cancer of the intestine. In certain embodiments, said disease or condition mediated by MALT1 is cancer of the colon. In certain embodiments, said disease or condition mediated by MALT1 is cancer of the rectum. In certain embodiments, said disease or condition mediated by MALT1 is cancer of the lung (e.g., non-small cell lung cancer or small cell lung cancer). In certain embodiments, said disease or condition mediated by MALT1 is cancer of the liver. In certain embodiments, said disease or condition mediated by MALT1 is cancer of the pancreas. In certain embodiments, said disease or condition mediated by MALT1 is cancer of the nerve. In certain embodiments, said disease or condition mediated by MALT1 is cancer of the brain (e.g., glioma or glioblastoma multiforme). In certain embodiments, said disease or condition mediated by MALT1 is cancer of the head and neck. In certain embodiments, said disease or condition mediated by MALT1 is cancer of the throat. In certain embodiments, said disease or condition mediated by MALT1 is cancer of the ovary. In certain embodiments, said disease or condition mediated by MALT1 is cancer of the uterus. In certain embodiments, said disease or condition mediated by MALT1 is cancer of the prostate. In certain embodiments, said disease or condition mediated by MALT1 is cancer of the testis. In certain embodiments, said disease or condition mediated by MALT1 is cancer of the bladder. In certain embodiments, said disease or condition mediated by MALT1 is cancer of the kidney. In certain embodiments, said disease or condition mediated by MALT1 is cancer of the breast. In certain embodiments, said disease or condition mediated by MALT1 is cancer of the gall bladder. In certain embodiments, said disease or condition mediated by MALT1 is cancer of the cervix. In certain embodiments, said disease or condition mediated by MALT1 is cancer of the thyroid. In certain embodiments, said disease or condition mediated by MALT1 is cancer of the prostate. In certain embodiments, said disease or condition mediated by MALT1 is cancer of the skin (e.g., skin squamous cell carcinoma). In certain embodiments, said disease or condition mediated by MALT1 is a solid tumor. In certain embodiments, said disease or condition mediated by MALT1 is gastric cancer. In certain embodiments, said disease or condition mediated by MALT1 is hepatocellular carcinoma. In certain embodiments, said disease or condition mediated by MALT1 is a peripheral nerve sheath tumor. In certain embodiments, said disease or condition mediated by MALT1 is pulmonary arterial hypertension.

In certain embodiments, the disease is a cancer associated with a viral infection. In certain embodiments, the disease is a cancer resulting from infection with an oncovirus. In certain embodiments, the oncovirus is hepatitis A, hepatitis B, hepatitis C, human T-lymphotropic virus (HTLV), human papillomavirus (HPV), Kaposi's sarcoma-associated herpesvirus (HHV-8), Merkel cell polyomavirus, or Epstein-Barr virus (EBV). In certain embodiments, the disease is human T-lymphotropic virus. In certain embodiments, the disease is Kaposi's sarcoma-associated herpesvirus. In certain embodiments, the disease is Epstein-Barr virus. Leukemias and lymphomas which may be associated with an oncoviral include: for HTLV, adult T-cell leukemia; for HHV-8, Castleman's disease and primary effusion lymphoma; and for EBV, Burkitt's lymphoma, Hogdkin's lymphoma, and post-transplant lymphoproliferative disease.

In certain embodiments, said disease or condition mediated by MALT1 is an inflammatory disorder or allergic disorder. In certain embodiments, said disease or condition mediated by MALT1 is an inflammatory disorder, such as autoimmune disorders, chronic inflammatory disorders, acute inflammatory disorders, auto-inflammatory disorders, fibrotic disorders, metabolic disorders, neoplasias, cardiovascular or cerebrovascular disorders, and myeloid cell-driven hyper-inflammatory response in COVID-19 infections. In certain embodiments, said disease or condition mediated by MALT1 is an allergic disorder, such as asthma and allergic rhinitis.

In certain embodiments, said disease or condition mediated by MALT1 is a disease or disorder of tissues and systemic disease [e.g., systemic lupus erythematosus (SLE); immune thrombocytopenic purpura (ITP); autoimmune hemolytic anemia (AHA); autoimmune neutropenia (AIN); Evans syndrome; proliferative and hyperproliferative diseases, such as cancer, atherosclerosis, rheumatoid arthritis, psoriasis, idiopathic pulmonary fibrosis, scleroderma, cirrhosis of the liver; and Acquired Immunodeficiency Syndrome (AIDS)]. In certain embodiments, said disease or condition mediated by MALTT is an immunologically-mediated disease, such as allograft rejection (e.g., rejection of transplanted organs or tissues). In certain embodiments, said disease or condition mediated by MALT1 is a tissue injury (e.g., associated with organ transplant or revascularization procedures). In certain embodiments, said disease or condition mediated by MALT1 is a disease or disorder of the respiratory tract (e.g., asthma). In certain embodiments, said disease or condition mediated by MALT1 is allergic rhinitis. In certain embodiments, said disease or condition mediated by MALT1 is a disease or disorder of the bone and joints (e.g., arthritis, rheumatoid arthritis). In certain embodiments, said disease or condition mediated by MALT1 is a disease or disorder of the skin. In certain embodiments, said disease or condition mediated by MALT1 is a disease or disorder of the gastrointestinal tract.

In certain embodiments, said disease or condition mediated by MALT1 is a reversible obstructive airways disease, such as asthma (e.g., bronchial asthma, allergic asthma, intrinsic asthma, extrinsic asthma, and dust asthma). In certain embodiments, said disease or condition mediated by MALT1 is chronic or inveterate asthma (e.g., late asthma airways hyper-responsiveness). In certain embodiments, said disease or condition mediated by MALT1 is bronchitis. In certain embodiments, said disease or condition mediated by MALT1 is a condition characterized by an inflammation of the nasal mucus membrane. In certain embodiments, said disease or condition mediated by MALT1 is acute rhinitis. In certain embodiments, said disease or condition mediated by MALT1 is allergic rhinitis. In certain embodiments, said disease or condition mediated by MALT1 is atrophic rhinitis. In certain embodiments, said disease or condition mediated by MALT1 is chronic rhinitis (e.g., rhinitis caseosa, hypertrophic rhinitis, rhinitis purulenta, rhinitis sicca, and rhinitis medicamentosa). In certain embodiments, said disease or condition mediated by MALT1 is membranous rhinitis (e.g., croupous rhinitis, fibrinous rhinitis, pseudomembranous rhinitis, and scrofoulous rhinitis). In certain embodiments, said disease or condition mediated by MALT1 is seasonal rhinitis [e.g., rhinitis nervosa (hay fever), vasomotor rhinitis, sarcoidosis, farmer's lung, and related diseases, such as fibroid lung and idiopathic interstitial pneumonia].

In certain embodiments, said disease or condition mediated by MALT1 includes pannus formation. In certain embodiments, said disease or condition mediated by MALT1 does not include pannus formation. In certain embodiments, said disease or condition mediated by MALT1 is rheumatoid arthritis. In certain embodiments, said disease or condition mediated by MALT1 is seronegative spondyloarthropathis (e.g., ankylosing spondylitis, psoriatic arthritis, and Reiter's disease). In certain embodiments, said disease or condition mediated by MALT1 is Behcet's disease. In certain embodiments, said disease or condition mediated by MALT1 is Sjogren's syndrome. In certain embodiments, said disease or condition mediated by MALT1 is systemic sclerosis.

In certain embodiments, said disease or condition mediated by MALT1 is psoriasis. In certain embodiments, said disease or condition mediated by MALT1 is systemic sclerosis. In certain embodiments, said disease or condition mediated by MALT1 is atopical dermatitis. In certain embodiments, said disease or condition mediated by MALT1 is contact dermatitis. In certain embodiments, said disease or condition mediated by MALT1 is eczematous dermatitis. In certain embodiments, said disease or condition mediated by MALT1 is seborrhoetic dermatitis. In certain embodiments, said disease or condition mediated by MALT1 is Lichen planus. In certain embodiments, said disease or condition mediated by MALT1 is Pemphigus. In certain embodiments, said disease or condition mediated by MALT1 is bullous Pemphigus. In certain embodiments, said disease or condition mediated by MALT1 is epidermolysis bullosa. In certain embodiments, said disease or condition mediated by MALT1 is urticaria. In certain embodiments, said disease or condition mediated by MALT1 is angiodermas. In certain embodiments, said disease or condition mediated by MALT1 is vasculitides. In certain embodiments, said disease or condition mediated by MALT1 is erythemas. In certain embodiments, said disease or condition mediated by MALT1 is cutaneous eosinophilias. In certain embodiments, said disease or condition mediated by MALT1 is uveitis. In certain embodiments, said disease or condition mediated by MALT1 is Alopecia. In certain embodiments, said disease or condition mediated by MALT1 is areata. In certain embodiments, said disease or condition mediated by MALT1 is vernal conjunctivitis.

In certain embodiments, said disease or condition mediated by MALT1 is Coeliac disease. In certain embodiments, said disease or condition mediated by MALT1 is proctitis. In certain embodiments, said disease or condition mediated by MALT1 is eosinophilic gastroenteritis. In certain embodiments, said disease or condition mediated by MALT1 is mastocytosis. In certain embodiments, said disease or condition mediated by MALT1 is pancreatitis. In certain embodiments, said disease or condition mediated by MALT1 is Crohn's disease. In certain embodiments, said disease or condition mediated by MALT1 is ulcerative colitis. In certain embodiments, said disease or condition mediated by MALT1 is a food-related allergy having effects remote from the gut (e.g., migraine, rhinitis, and eczema).

In certain embodiments, said disease or condition mediated by MALT1 is multiple sclerosis. In certain embodiments, said disease or condition mediated by MALT1 is artherosclerosis. In certain embodiments, said disease or condition mediated by MALT1 is acquired immunodeficiency syndrome (AIDS). In certain embodiments, said disease or condition mediated by MALT1 is lupus. In certain embodiments, said disease or condition mediated by MALT1 is lupus erythematosus. In certain embodiments, said disease or condition mediated by MALT1 is systemic lupus erythenatosus. In certain embodiments, said disease or condition mediated by MALT1 is Hashimoto's thyroiditis. In certain embodiments, said disease or condition mediated by MALT1 is myasthenia gravis. In certain embodiments, said disease or condition mediated by MALT1 is type I diabetes. In certain embodiments, said disease or condition mediated by MALT1 is nephrotic syndrome. In certain embodiments, said disease or condition mediated by MALT1 is eosinophilia fasciitis. In certain embodiments, said disease or condition mediated by MALT1 is hyper IgE syndrome. In certain embodiments, said disease or condition mediated by MALT1 is lepromatous leprosy. In certain embodiments, said disease or condition mediated by MALT1 is sezary syndrome. In certain embodiments, said disease or condition mediated by MALT1 is idiopathic thrombocytopenia purpura. In certain embodiments, said disease or condition mediated by MALT1 is restenosis following angioplasty. In certain embodiments, said disease or condition mediated by MALT1 is a tumor (e.g., leukemia, lymphomas). In certain embodiments, said disease or condition mediated by MALT1 is artherosclerosis.

In certain embodiments, said disease or condition mediated by MALT1 is acute chronic allograft rejection (e.g., following transplantation of kidney, heart, liver, lung, bone marrow, skin, or cornea). In certain embodiments, said disease or condition mediated by MALT1 is chronic allograft rejection (e.g., following transplantation of kidney, heart, liver, lung, bone marrow, skin, or cornea). In certain embodiments, said disease or condition mediated by MALT1 is chronic graft-versus-host disease.

In certain embodiments, said disease or condition mediated by MALT1 is an acute inflammatory disorder. In certain embodiments, said disease or condition mediated by MALT1 is an auto-inflammatory disorder. In certain embodiments, said disease or condition mediated by MALT1 is a fibrotic disorder. In certain embodiments, said disease or condition mediated by MALT1 is a metabolic disorder. In certain embodiments, said disease or condition mediated by MALT1 is a neoplasia. In certain embodiments, said disease or condition mediated by MALT1 is a cardiovascular or cerebrovascular disorder. In certain embodiments, said disease or condition mediated by MALT1 is a myeloid cell-driven hyper-inflammatory response in COVID-19 infections.

In certain embodiments, said disease or condition mediated by MALT1 is an autoimmune disorder. In certain embodiments, said disease or condition mediated by MALT1 is a chronic inflammatory disorder. In certain embodiments, said disease or condition mediated by MALT1 is an acute inflammatory disorder. In certain embodiments, said disease or condition mediated by MALT1 is an auto-inflammatory disorder. In certain embodiments, said disease or condition mediated by MALT1 is a combination of one, two, or all three of a chronic inflammatory disorder, an acute inflammatory disorder, and an auto-inflammatory disorder.

In certain embodiments, said disease or condition mediated by MALT1 is an inflammatory bowel disease (e.g., ulcerative colitis or Crohn's disease). In certain embodiments, said disease or condition mediated by MALT1 is multiple sclerosis. In certain embodiments, said disease or condition mediated by MALT1 is psoriasis. In certain embodiments, said disease or condition mediated by MALT1 is arthritis. In certain embodiments, said disease or condition mediated by MALT1 is rheumatoid arthritis. In certain embodiments, said disease or condition mediated by MALT1 is osteoarthritis. In certain embodiments, said disease or condition mediated by MALT1 is juvenile arthritis. In certain embodiments, said disease or condition mediated by MALT1 is psoriatic arthritis. In certain embodiments, said disease or condition mediated by MALT1 is reactive arthritis. In certain embodiments, said disease or condition mediated by MALT1 is ankylosing spondylitis. In certain embodiments, said disease or condition mediated by MALT1 is cryopyrin-associated periodic syndromes. In certain embodiments, said disease or condition mediated by MALT1 is Muckle-Wells syndrome. In certain embodiments, said disease or condition mediated by MALT1 is familial cold auto-inflammatory syndrome. In certain embodiments, said disease or condition mediated by MALT1 is neonatal-onset multisystem inflammatory disease. In certain embodiments, said disease or condition mediated by MALT1 is TNF receptor-associated periodic syndrome. In certain embodiments, said disease or condition mediated by MALT1 is acute and chronic pancreatitis. In certain embodiments, said disease or condition mediated by MALT1 is atherosclerosis. In certain embodiments, said disease or condition mediated by MALT1 is gout. In certain embodiments, said disease or condition mediated by MALT1 is a fibrotic disorder (e.g., hepatic fibrosis or idiopathic pulmonary fibrosis). In certain embodiments, said disease or condition mediated by MALT1 is nephropathy. In certain embodiments, said disease or condition mediated by MALT1 is sarcoidosis. In certain embodiments, said disease or condition mediated by MALT1 is scleroderma. In certain embodiments, said disease or condition mediated by MALT1 is anaphylaxis. In certain embodiments, said disease or condition mediated by MALT1 is diabetes (e.g., diabetes mellitus type 1 or diabetes mellitus type 2). In certain embodiments, said disease or condition mediated by MALT1 is diabetic retinopathy. In certain embodiments, said disease or condition mediated by MALT1 is Still's disease. In certain embodiments, said disease or condition mediated by MALT1 is vasculitis. In certain embodiments, said disease or condition mediated by MALT1 is sarcoidosis. In certain embodiments, said disease or condition mediated by MALT1 is pulmonary inflammation. In certain embodiments, said disease or condition mediated by MALT1 is respiratory failure. In certain embodiments, said disease or condition mediated by MALT1 is acute respiratory distress syndrome. In certain embodiments, said disease or condition mediated by MALT1 is chronic eosinophilic pneumonia. In certain embodiments, said disease or condition mediated by MALT1 is wet and dry age-related macular degeneration. In certain embodiments, said disease or condition mediated by MALT1 is autoimmune hemolytic syndromes. In certain embodiments, said disease or condition mediated by MALT1 is autoimmune and inflammatory hepatitis. In certain embodiments, said disease or condition mediated by MALT1 is autoimmune neuropathy. In certain embodiments, said disease or condition mediated by MALT1 is autoimmune ovarian failure. In certain embodiments, said disease or condition mediated by MALT1 is autoimmune orchitis. In certain embodiments, said disease or condition mediated by MALT1 is autoimmune thrombocytopenia. In certain embodiments, said disease or condition mediated by MALT1 is silicone implant-associated autoimmune disease. In certain embodiments, said disease or condition mediated by MALT1 is Sjogren's syndrome. In certain embodiments, said disease or condition mediated by MALT1 is familial Mediterranean fever. In certain embodiments, said disease or condition mediated by MALT1 is systemic lupus erythematosus. In certain embodiments, said disease or condition mediated by MALT1 is vasculitis syndromes (e.g., temporal, Takayasu's and giant cell arteritis, Behcet's disease or Wegener's granulomatosis). In certain embodiments, said disease or condition mediated by MALT1 is vitiligo. In certain embodiments, said disease or condition mediated by MALT1 is secondary hematologic manifestation of autoimmune diseases (e.g., anemias). In certain embodiments, said disease or condition mediated by MALT1 is drug-induced autoimmunity. In certain embodiments, said disease or condition mediated by MALT1 is Hashimoto's thyroiditis. In certain embodiments, said disease or condition mediated by MALT1 is hypophysitis. In certain embodiments, said disease or condition mediated by MALT1 is idiopathic thrombocytic pupura. In certain embodiments, said disease or condition mediated by MALT1 is metal-induced autoimmunity. In certain embodiments, said disease or condition mediated by MALT1 is myasthenia gravis. In certain embodiments, said disease or condition mediated by MALT1 is pemphigus. In certain embodiments, said disease or condition mediated by MALT1 is autoimmune deafness (e.g., Meniere's disease). In certain embodiments, said disease or condition mediated by MALT1 is Goodpasture's syndrome. In certain embodiments, said disease or condition mediated by MALT1 is Graves' disease. In certain embodiments, said disease or condition mediated by MALT1 is an HW-related autoimmune syndromes. In certain embodiments, said disease or condition mediated by MALT1 is Gullain-Barre disease. In certain embodiments, said disease or condition mediated by MALT1 is Addison's disease. In certain embodiments, said disease or condition mediated by MALT1 is anti-phospholipid syndrome. In certain embodiments, said disease or condition mediated by MALT1 is asthma. In certain embodiments, said disease or condition mediated by MALT1 is atopic dermatitis. In certain embodiments, said disease or condition mediated by MALT1 is Celiac disease. In certain embodiments, said disease or condition mediated by MALT1 is Cushing's syndrome. In certain embodiments, said disease or condition mediated by MALT1 is dermatomyositis. In certain embodiments, said disease or condition mediated by MALT1 is idiopathic adrenal atrophy. In certain embodiments, said disease or condition mediated by MALT1 is idiopathic thrombocytopenia. In certain embodiments, said disease or condition mediated by MALT1 is Kawasaki syndrome. In certain embodiments, said disease or condition mediated by MALT1 is Lambert-Eaton Syndrome. In certain embodiments, said disease or condition mediated by MALT1 is pemicious anemia. In certain embodiments, said disease or condition mediated by MALT1 is pollinosis. In certain embodiments, said disease or condition mediated by MALT1 is polyarteritis nodosa. In certain embodiments, said disease or condition mediated by MALT1 is primary biliary cirrhosis. In certain embodiments, said disease or condition mediated by MALT1 is primary sclerosing cholangitis. In certain embodiments, said disease or condition mediated by MALT1 is Raynaud's disease. In certain embodiments, said disease or condition mediated by MALT1 is Raynaud's phenomenon. In certain embodiments, said disease or condition mediated by MALT1 is Reiter's Syndrome. In certain embodiments, said disease or condition mediated by MALT1 is relapsing polychondritis. In certain embodiments, said disease or condition mediated by MALT1 is Schmidt's syndrome. In certain embodiments, said disease or condition mediated by MALT1 is thyrotoxidosis. In certain embodiments, said disease or condition mediated by MALT1 is sepsis. In certain embodiments, said disease or condition mediated by MALT1 is septic shock. In certain embodiments, said disease or condition mediated by MALT1 is endotoxic shock. In certain embodiments, said disease or condition mediated by MALT1 is exotoxin-induced toxic shock. In certain embodiments, said disease or condition mediated by MALT1 is gram negative sepsis. In certain embodiments, said disease or condition mediated by MALT1 is toxic shock syndrome. In certain embodiments, said disease or condition mediated by MALT1 is glomerulonephritis. In certain embodiments, said disease or condition mediated by MALT1 is peritonitis. In certain embodiments, said disease or condition mediated by MALT1 is interstitial cystitis. In certain embodiments, said disease or condition mediated by MALT1 is hyperoxia-induced inflammations. In certain embodiments, said disease or condition mediated by MALT1 is chronic obstructive pulmonary disease (COPD). In certain embodiments, said disease or condition mediated by MALT1 is emphysema. In certain embodiments, said disease or condition mediated by MALT1 is nasal inflammation. In certain embodiments, said disease or condition mediated by MALT1 is vasculitis. In certain embodiments, said disease or condition mediated by MALT1 is graft vs. host reaction (e.g., graft vs. host disease). In certain embodiments, said disease or condition mediated by MALT1 is allograft rejections (e.g., acute allograft rejection or chronic allograft rejection). In certain embodiments, said disease or condition mediated by MALT1 is early transplantation rejection (e.g., acute allograft rejection). In certain embodiments, said disease or condition mediated by MALT1 is reperfusion injury. In certain embodiments, said disease or condition mediated by MALT1 is pain (e.g., acute pain, chronic pain, neuropathic pain, or fibromyalgia). In certain embodiments, said disease or condition mediated by MALT1 is a chronic infection. In certain embodiments, said disease or condition mediated by MALT1 is meningitis. In certain embodiments, said disease or condition mediated by MALT1 is encephalitis. In certain embodiments, said disease or condition mediated by MALT1 is myocarditis. In certain embodiments, said disease or condition mediated by MALT1 is gingivitis. In certain embodiments, said disease or condition mediated by MALT1 is post-surgical trauma. In certain embodiments, said disease or condition mediated by MALT1 is tissue injury. In certain embodiments, said disease or condition mediated by MALT1 is traumatic brain injury. In certain embodiments, said disease or condition mediated by MALT1 is enterocolitis. In certain embodiments, said disease or condition mediated by MALT1 is sinusitis. In certain embodiments, said disease or condition mediated by MALT1 is uveitis. In certain embodiments, said disease or condition mediated by MALT1 is ocular inflammation. In certain embodiments, said disease or condition mediated by MALT1 is optic neuritis. In certain embodiments, said disease or condition mediated by MALT1 is gastric ulcers. In certain embodiments, said disease or condition mediated by MALT1 is esophagitis. In certain embodiments, said disease or condition mediated by MALT1 is peritonitis. In certain embodiments, said disease or condition mediated by MALT1 is periodontitis. In certain embodiments, said disease or condition mediated by MALT1 is dermatomyositis. In certain embodiments, said disease or condition mediated by MALT1 is gastritis. In certain embodiments, said disease or condition mediated by MALT1 is myositis. In certain embodiments, said disease or condition mediated by MALT1 is polymyalgia. In certain embodiments, said disease or condition mediated by MALT1 is pneumonia. In certain embodiments, said disease or condition mediated by MALT1 is bronchitis. In certain embodiments, the disease or condition mediated by MALT1 is endometriosis. In certain embodiments, the disease or condition mediated by MALT1 is necrotizing vasculitis. In certain embodiments, the disease or condition mediated by MALT1 is lymphadenitis. In certain embodiments, the disease or condition mediated by MALT1 is peri-arteritis nodosa. In certain embodiments, the disease or condition mediated by MALT1 is anti-phospholipid antibody syndrome. In certain embodiments, the disease or condition mediated by MALT1 is pemphigus vulgaris. In certain embodiments, the disease or condition mediated by MALT1 is Lyme disease. In certain embodiments, the disease or condition mediated by MALT1 is cardiomyopathy. In certain embodiments, the disease or condition mediated by MALT1 is rheumatic fever. In certain embodiments, the disease or condition mediated by MALT1 is a blistering disorder. In certain embodiments, the disease or condition mediated by MALT1 is an antibody-mediated vasculitis syndrome. In certain embodiments, the disease or condition mediated by MALT1 is an immune-complex vasculitide. In certain embodiments, the disease or condition mediated by MALT1 i, oedema. In certain embodiments, the disease or condition mediated by MALT1 is embolism. In certain embodiments, the disease or condition mediated by MALT1 is fibrosis. In certain embodiments, the disease or condition mediated by MALT1 is silicosis. In certain embodiments, the disease or condition mediated by MALT1 is BENTA disease. In certain embodiments, the disease or condition mediated by MALT1 is berylliosis.

In certain embodiments, said disease or condition mediated by MALT1 is systemic sclerosis/scleroderma. In certain embodiments, said disease or condition mediated by MALT1 is lupus nephritis. In certain embodiments, said disease or condition mediated by MALT1 is connective tissue disease. In certain embodiments, said disease or condition mediated by MALT1 is wound healing. In certain embodiments, said disease or condition mediated by MALT1 is surgical scarring. In certain embodiments, said disease or condition mediated by MALT1 is spinal cord injury. In certain embodiments, said disease or condition mediated by MALT1 is CNS scarring. In certain embodiments, said disease or condition mediated by MALT1 is acute lung injury. In certain embodiments, said disease or condition mediated by MALT1 is pulmonary fibrosis (e.g., idiopathic pulmonary fibrosis or cystic fibrosis). In certain embodiments, said disease or condition mediated by MALT1 is chronic obstructive pulmonary disease. In certain embodiments, said disease or condition mediated by MALT1 is adult respiratory distress syndrome. In certain embodiments, said disease or condition mediated by MALT1 is acute lung injury. In certain embodiments, said disease or condition mediated by MALT1 is drug-induced lung injury. In certain embodiments, said disease or condition mediated by MALT1 is glomerulonephritis. In certain embodiments, said disease or condition mediated by MALT1 is chronic kidney disease (e.g., diabetic nephropathy). In certain embodiments, said disease or condition mediated by MALT1 is hypertension-induced nephropathy. In certain embodiments, said disease or condition mediated by MALT1 is alimentary track or gastrointestinal fibrosis. In certain embodiments, said disease or condition mediated by MALT1 is renal fibrosis. In certain embodiments, said disease or condition mediated by MALT1 is hepatic or biliary fibrosis. In certain embodiments, said disease or condition mediated by MALT1 is liver fibrosis (e.g., nonalcoholic steatohepatitis, hepatitis C, or hepatocellular carcinoma). In certain embodiments, said disease or condition mediated by MALT1 is cirrhosis (e.g., primary biliary cirrhosis or cirrhosis due to fatty liver disease, such as alcoholic and nonalcoholic steatosis). In certain embodiments, said disease or condition mediated by MALT1 is radiation-induced fibrosis (e.g., head and neck, gastrointestinal or pulmonary). In certain embodiments, said disease or condition mediated by MALT1 is primary sclerosing cholangitis. In certain embodiments, said disease or condition mediated by MALT1 is restenosis. In certain embodiments, said disease or condition mediated by MALT1 is cardiac fibrosis (e.g., endomyocardial fibrosis or atrial fibrosis). In certain embodiments, said disease or condition mediated by MALT1 is opthalmic scarring. In certain embodiments, said disease or condition mediated by MALT1 is fibrosclerosis. In certain embodiments, said disease or condition mediated by MALT1 is a fibrotic cancer. In certain embodiments, said disease or condition mediated by MALT1 is fibroids. In certain embodiments, said disease or condition mediated by MALT1 is fibroma. In certain embodiments, said disease or condition mediated by MALT1 is a fibroadenoma In certain embodiments, said disease or condition mediated by MALT1 is a fibrosarcoma. In certain embodiments, said disease or condition mediated by MALT1 is transplant arteriopathy. In certain embodiments, said disease or condition mediated by MALT1 is keloid. In certain embodiments, said disease or condition mediated by MALT1 is mediastinal fibrosis. In certain embodiments, said disease or condition mediated by MALT1 is myelofibrosis. In certain embodiments, said disease or condition mediated by MALT1 is retroperitoneal fibrosis. In certain embodiments, said disease or condition mediated by MALT1 is progressive massive fibrosis. In certain embodiments, said disease or condition mediated by MALT1 is nephrogenic systemic fibrosis.

In certain embodiments, said disease or condition mediated by MALT1 is obesity. In certain embodiments, said disease or condition mediated by MALT1 is steroid-resistance. In certain embodiments, said disease or condition mediated by MALT1 is glucose intolerance. In certain embodiments, said disease or condition mediated by MALT1 is metabolic syndrome.

In certain embodiments, said disease or condition mediated by MALT1 is atherosclerosis. In certain embodiments, said disease or condition mediated by MALT1 is restenosis of an atherosclerotic coronary artery. In certain embodiments, said disease or condition mediated by MALT1 is acute coronary syndrome. In certain embodiments, said disease or condition mediated by MALT1 is myocardial infarction. In certain embodiments, said disease or condition mediated by MALT1 is cardiac-allograft vasculopathy. In certain embodiments, said disease or condition mediated by MALT1 is stroke. In certain embodiments, said disease or condition mediated by MALT1 is a central nervous system disorder with an inflammatory or apoptotic component. In certain embodiments, said disease or condition mediated by MALT1 is Alzheimer's disease. In certain embodiments, said disease or condition mediated by MALT1 is Parkinson's disease. In certain embodiments, said disease or condition mediated by MALT1 is Huntington's disease. In certain embodiments, said disease or condition mediated by MALT1 is amyotrophic lateral sclerosis. In certain embodiments, said disease or condition mediated by MALT1 is spinal cord injury. In certain embodiments, said disease or condition mediated by MALT1 is neuronal ischemia. In certain embodiments, said disease or condition mediated by MALT1 is peripheral neuropathy.

In certain embodiments, said disease or condition mediated by MALT1 is a disease or disorder associated with a coronavirus (e.g. SARS-CoV-2). In certain embodiments, said coronavirus is SARS-CoV-2. In certain embodiments, the disease or disorder associated with SARS-CoV-2 is COVID-19.

In certain embodiments, the disease or condition mediated by MALT1 is a rheumatic disease. In certain embodiments, the disease or condition mediated by MALT1 is an inflammatory arthropathy. In certain embodiments, the disease or condition mediated by MALT1 is rheumatoid arthritis, juvenile arthritis, Still's disease, juvenile rheumatoid arthritis, systemic onset rheumatoid arthritis, pauciarticular rheumatoid arthritis, pauciarticular juvenile rheumatoid arthritis, polyarticular rheumatoid arthritis, enteropathic arthritis, juvenile Reiter's Syndrome, ankylosing spondylitis, juvenile ankylosing spondylitis, SEA Syndrome, reactive arthritis (reactive arthropathy), psoriatic arthropathy, juvenile enteropathic arthritis, polymyalgia rheumatica, enteropathic spondylitis, juvenile Idiopathic Arthritis (JIA), juvenile psoriatic arthritis, juvenile rheumatoid arthritis, systemic onset juvenile rheumatoid arthritis, giant cell arteritis, secondary osteoarthritis from an inflammatory disease.

In certain embodiments, the disease or condition mediated by MALT1 is a connective tissue disease. In certain embodiments, the disease or condition mediated by MALT1 is lupus, systemic lupus erythematosus, juvenile systemic lupus erythematosus, nephritis, Sjögren's syndrome, scleroderma (systemic sclerosis), Raynaud's phenomenonjuvenile scleroderma, polymyositis, dermatomyositis, polymyositis-dermatomyositis, polymyalgia rheumatica, a mixed connective tissue disease, sarcoidosis, fibromyalgia, vasculitis microscopic polyangiitis, vasculitis, eosinophilic granulomatosis with polyangiitis (formerly known as Churg-Strauss Syndrome), granulomatosis with polyangiitis (formerly known as Wegener's granulomatosis), polyarteritis nodosa, Henoch-Schonlein purpura, idiopathic thrombocytopenic thrombotic purpura, juvenile vasculitis, polyarteritis nodossa (also known as panarteritis nodosa, periarteritis nodosa Kussmaul disease, Kussmaul-Maier disease or PAN), serum sickness, myasthenia gravis, Takayasu's arteritis, Behçet's syndrome, Kawasaki's disease (mucocutaneous lymph node syndrome), Buerger's disease (thromboangiitis obliterans), Vogt-Koyanagi-Harada syndrome, Addison's disease, Hashimoto's thyroiditis, primary biliary sclerosis, autoimmune hepatitis, chronic aggressive hepatitis, nonalcoholic hepatic steatosis, sclerosing cholangitis, membranous glomerulopathy, polymyositis, myositis, atherosclerosis, autoimmune hemolytic anemia, autoimmune orchitis, Goodpasture's disease,

In certain embodiments, the disease or condition mediated by MALT1 is a neurodegenerative disease or neuroinflammatory disease. In certain embodiments, the disease or condition mediated by MALT1 is multiple sclerosis, amyotropic lateral sclerosis, Guillain-Barre disease, autoimmune encephalomyelitis, Alzheimer's disease, major depressive disorder, traumatic brain injury, epilepsy, Parkinson's disease, or bipolar disorder.

In certain embodiments, the disease or condition mediated by MALT1 is an inflammatory bowel disease. In certain embodiments, the disease or condition mediated by MALT1 is Crohn's disease, ulcerative colitis, Celiac Sprue, Celiac disease, proctitis, eosinophilic gastroenteritis, autoimmune atrophic gastritis of pernicious anemia, or mastocytosis.

In certain embodiments, the disease or condition mediated by MALT1 is a skin autoimmune disorder. In certain embodiments, the disease or condition mediated by MALT1 is psoriasis. In certain embodiments, the disease or condition mediated by MALT1 is eczema. In certain embodiments, the disease or condition mediated by MALT1 is plaque psoriasis, Guttate psoriasis, psoriatic epidermal hyperplasia, inverse psoriasis, pustular psoriasis, erythrodermic psoriasis, atopic dermatitis, eczema dermatitis, dermatitis, rosacea, pruritus, alopecia areata, vitiligo, epidermal hyperplasia, juvenile dermatomyositis, dermatomyositis, or hidradenitis suppurativa.

In certain embodiments, the disease or condition mediated by MALT1 is an organ or cell transplant rejection. In certain embodiments, the disease or condition mediated by MALT1 is graft-versus-host disease. In certain embodiments, the disease or condition mediated by MALT1 is chronic graft-versus-host disease, acute graft-versus-host disease, or organ or cell transplant rejection such as bone marrow, cartilage, cornea, heart, intervertebral disc, islet, kidney, limb, liver, lung, muscle, myoblast, nerve, pancreas, skin, small intestine, or trachea, or xeno transplantation.

In certain embodiments, the disease or condition mediated by MALT1 is an autoimmune disease of the eye. In certain embodiments, the disease or condition mediated by MALT1 is Graves' disease, noninfectious uveitis, dry eye syndrome, sympathetic ophthalmia, Cogan's syndrome, keratoconjunctivitis, vernal conjunctivitis, uveitis (e.g., uveitis associated with Behcet's disease and lens-induced uveitis), keratitis, herpetic keratitis, conical keratitis, corneal epithelial dystrophy, keratoleukoma, ocular premphigus, Mooren's ulcer, scleritis, keratoconjunctivitis sicca (dry eye), phlyctenule, iridocyclitis, sarcoidosis, endocrine ophthalmopathy, sympathetic ophthalmitis, allergic conjunctivitis, or ocular neovascularization

In certain embodiments, the disease or condition mediated by MALT1 is an ocular manifestation of an autoimmune disease.

In certain embodiments, the disease or condition mediated by MALT1 is a respiratory disease. In certain embodiments, the disease or condition mediated by MALT1 is asthma, chronic obstructive pulmonary disease, or acute respiratory disease.

In certain embodiments, the disease or condition mediated by MALT1 is diabetes. In certain embodiments, the disease or condition mediated by MALT1 is Type I diabetes mellitus, Type II diabetes mellitus, or juvenile onset diabetes.

### Additional Methods

Another aspect of the invention provides methods of inhibiting cell proliferation in a subject by administering to the subject a compound described herein (e.g., a compound of Formula I or I-1), or inhibiting cell proliferation in a biological sample by contacting the biological sample with a compound described herein (e.g., a compound of Formula I or I-1). In certain embodiments, the compound is a compound of Formula I, Ia, Ib, Ic, Id, Ie, If, Ig, Ih, Ii, Ij, Ik, II, Im, In, Io, Ip, Iq, Ir, Is, It, Iu, Iv, Iw, Ix, Iy, Iz, I-1, Ia-1, Tb-1, Ic-1, Id-1, Ie-1, If-1, Ig-1. Ih-1, Ii-1, Ij-1, Ik-1, Il-1, Im-1, In-1, Io-1, Ip-1, Iq-1, Ir-1, Is-1, It-1, Iu-1, Iv-1, Iw-1, or Ix-1, or other compounds in Section I, or defined by one of the embodiments described above. In certain embodiments, cell proliferation is inhibited for T-cells. In certain embodiments, cell proliferation is inhibited for B-cells. In certain embodiments, cell proliferation is inhibited for T-cells and B-cells.

Another aspect of the invention provides methods of inducing apoptosis of a cell in a subject by administering to the subject a compound described herein (e.g., a compound of Formula I or I-1), or inducing apoptosis of a cell in a biological sample by contacting the biological sample with a compound described herein (e.g., a compound of Formula I or I-1). In certain embodiments, the compound is a compound of Formula I, Ia, Ib, Ic, Id, Ie, If, Ig, Ih, Ii, Ij, Ik, Ii, Im, In, Io, Ip, Iq, Ir, Is, It, Iu, Iv, Iw, Ix, Iy, Iz, I-1, Ia-1, Ib-1, Ic-1, Id-1, Ie-1, If-1, Ig-1, Ih-1, Ii-1, Ij-1, Ik-1, Il-1, Im-1, In-1, Io-1, Ip-1, Iq-1, Ir-1, Is-1, It-1, Iu-1, Iv-1, Iw-1, or Ix-1, or other compounds in Section I, or defined by one of the embodiments described above. In certain embodiments, cell is a tumor cell. In certain embodiments, the cell is a lymphocyte. In certain embodiments, the cell is a T-cell. In certain embodiments, the cell is a B-cell.

Another aspect of the invention provides methods of inhibiting adhesion of a cell in a subject by administering to the subject a compound described herein (e.g., a compound of Formula I or I-1), or inhibiting adhesion of a cell in a biological sample by contacting the biological sample with a compound described herein (e.g., a compound of Formula I or I-1). In certain embodiments, the compound is a compound of Formula I, Ia, Ib, Ic, Id, Ie, If, Ig, Ih, Ii, Ij, Ik, Ii, Im, In, Io, Ip, Iq, Ir, Is, It, Iu, Iv, Iw, Ix, Iy, Iz, I-1, Ia-1, Ib-1, Ic-1, Id-1, Ie-1, If-1, Ig-1, Ih-1, Ii-1, Ij-1, Ik-1, Il-1, Im-1, In-1, Io-1, Ip-1, Iq-1, Ir-1, Is-1, It-1, Iu-1, Iv-1, Iw-1, or Ix-1, or other compounds in Section I, or defined by one of the embodiments described above. In certain embodiments, the cell is a tumor cell. In certain embodiments, the cell is a lymphocyte. In certain embodiments, the cell is a T-cell. In certain embodiments, the cell is a B-cell.

Another aspect of the invention provides methods of inhibiting activation of T-cells or B-cells in a subject by administering to the subject a compound described herein (e.g., a compound of Formula I or I-1), or inhibiting activation of T-cells or B-cells in a biological sample by contacting the biological sample with a compound described herein (e.g., a compound of Formula I or I-1). In certain embodiments, the compound is a compound of Formula I, Ia, Ib, Ic, Id, Ie, If, Ig, Ih, Ii, Ij, Ik, Ii, Im, In, Io, Ip, Iq, Ir, Is, It, Iu, Iv, Iw, Ix, Iy, Iz, I-1, Ia-1, Ib-1, Ic-1, Id-1, Ie-1, If-1, Ig-1, Ih-1, Ii-1, Ij-1, Ik-1. Il-1, Im-1, In-1, Io-1, Ip-1, Iq-1, Ir-1, Is-1, It-1, Iu-1, Iv-1, Iw-1, or Ix-1, or other compounds in Section I, or defined by one of the embodiments described above.

Another aspect of the invention provides methods of inhibiting the activity of mucosa-associated lymphoid tissue lymphoma translation protein 1 (MALT1) or a MALT1 fusion protein in a subject by administering to the subject a compound described herein (e.g., a compound of Formula I or I-1), or inhibiting the activity of mucosa-associated lymphoid tissue lymphoma translation protein 1 (MALT1) or a MALT1 fusion protein in a biological sample by contacting the biological sample with a compound described herein (e.g., a compound of Formula I or I-1). In certain embodiments, the compound is a compound of Formula I, Ia, Ib, Ic, Id, Ie, If, Ig, Ih, Ii, Ij, Ik. Ii, Im, In, Io, Ip, Iq, Ir, Is, It, Iu, Iv, Iw, Ix, Iy, Iz, I-1, Ia-1, Ib-1, Ic-1, Id-1, Ie-1, If-1, Ig-1, Ih-1, Ii-1, Ij-1, Ik-1, Il-1, Im-1, In-1, Io-1, Ip-1, Iq-1, Ir-1, Is-1, It-1, Iu-1, Iv-1, Iw-1, or Ix-1, or other compounds in Section I, or defined by one of the embodiments described above. In certain embodiments, the method inhibits the protease activity of MALT1. In certain embodiments, the method inhibits the protease activity of a MALT1 fusion protein (e.g., API2-MALT1). In certain embodiments, the method inhibits the protease activity of MALT1 or a MALT1 fusion protein for cleavage of a peptide substrate. In certain embodiments, the peptide substrate is A20, Bc110, RelB, CYLD, NIK, regnase-1, roquin-1, roquin-2, LIMA1a, or MALT1. The inhibitor may selectively inhibit the protease activity of MALT1 or a MALT1 fusion protein for cleavage of a first peptide substrate over protease activity for cleavage of a second peptide substrate. In certain embodiments, the first and/or second substrate is A20, Bcl10, RelB, CYLD, NIK, regnase-1, roquin-1, roquin-2, LIMA1a, or MALT1. In certain embodiments, the selectivity is between about 1.25 fold and about 5 fold. In certain embodiments, the selectivity is between about 5 fold and about 10 fold. In certain embodiments, the selectivity is between about 10 fold and about 25 fold. In certain embodiments, the selectivity is between about 25 fold and about 50 fold. In certain embodiments, the selectivity is between about 50 fold and about 100 fold. In certain embodiments, the selectivity is between about 100 fold and about 250 fold. In certain embodiments. In certain embodiments, the selectivity is between about 250 fold and about 500 fold. In certain embodiments, the selectivity is between about 500 fold and about 1000 fold. In certain embodiments, or at least about 1000 fold.

### III. Combination Therapy

Another aspect of the invention provides for combination therapy. Isothiazolylcarboxamide compounds described herein (e.g., a compound of Formula I, Ia, Ib, Ic, Id, Ie, If, Ig, Ih, Ii, Ij, Ik, II, Im, In, Io, Ip, Iq, Ir, Is, It, Iu, Iv, Iw, Ix, Iy, Iz, I-1, Ia-1, Ib-1, Ic-1, Id-1, Ie-1, If-1, Ig-1, Ih-1, Ii-1, Ij-1, Ik-1, Il-1, Im-1, In-1, Io-1, Ip-1, Iq-1, Ir-1, Is-1, It-1, Iu-1, Iv-1, Iw-1, or Ix-1, or other compounds in Section I) or their pharmaceutically acceptable salts may be used in combination with additional therapeutic agents to treat diseases or conditions, such as an inflammatory disorder.

Accordingly, in some embodiments, the present invention provides a method of treating a disclosed disease or condition comprising administering to a patient in need thereof an effective amount of a compound disclosed herein and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents.

One or more other therapeutic agents may be administered separately from a compound or composition of the invention, as part of a multiple dosage regimen. Alternatively, one or more other therapeutic agents may be part of a single dosage form, mixed together with a compound of this invention in a single composition. If administered as a multiple dosage regime, one or more other therapeutic agent and a compound or composition of the invention may be administered simultaneously, sequentially or within a period of time from one another.

In certain embodiments, the compounds of the disclosure can be administered with one or more of a second therapeutic agent, sequentially or concurrently, either by the same route or by different routes of administration. When administered sequentially, the time between administrations is selected to benefit, among others, the therapeutic efficacy and/or safety of the combination treatment. In certain embodiments, the compound of the disclosure can be administered first followed by a second therapeutic agent, or alternatively, the second therapeutic agent administered first followed by the compound of the disclosure. In certain embodiments, the compound of the disclosure can be administered for the same duration as the second therapeutic agent, or alternatively, for a longer or shorter duration as the second therapeutic compound.

When administered concurrently, the compounds of the disclosure can be administered separately at the same time as the second therapeutic agent, by the same or different routes, or administered in a single composition by the same route. In certain embodiments, the compound of the disclosure is prepared as a first pharmaceutical composition, and the second therapeutic agent prepared as a second pharmaceutical composition, where the first pharmaceutical composition and the second pharmaceutical composition are administered simultaneously, sequentially, or separately. In certain embodiments, the amount and frequency of administration of the second therapeutic agent can used standard dosages and standard administration frequencies used for the particular therapeutic agent. See, e.g., Physicians' Desk Reference, 70th Ed., PDR Network, 2015; incorporated herein by reference.

In certain embodiments, the additional therapeutic agent is a leukotriene inhibitor, non-steroidal anti-inflammatory drug (NSAID), steroid, tyrosine kinase inhibitor, receptor kinase inhibitor, modulator of nuclear receptor family of transcription factor, HSP90 inhibitor, adenosine receptor (A₂A) agonist, disease modifying antirheumatic drugs (DMARDS), phosphodiesterase (PDE) inhibitor, neutrophil elastase inhibitor, modulator of Axl kinase, an anti-cancer agent, anti-allergic agent, anti-nausea agent (or anti-emetic), pain reliever, cytoprotective agent, or a combination thereof. In certain embodiments, the additional therapeutic agent is an anti-cancer agent, an analgesic, an anti-inflammatory agent, or a combination thereof.

In certain embodiments, the second therapeutic agent is a leukotriene inhibitor. Examples of leukotriene inhibitors considered for use in combination therapies of the invention include but are not limited to montelukast, zafirlukast, pranlukast, zileuton, or combinations thereof.

In certain embodiments, the second therapeutic agent is a an NSAID. Examples of NSAIDs considered for use in combination therapies of the invention include but are not limited to acetylsalicylic acid, diflunisal, salsalate, ibuprofen, dexibuprofen, naioxen, fenoprofen, ketoprofen, dexketoprofen, flurbiprofen, oxaprozin, loxoprofen, indomethacin, tolmetin, sulindac, etodolac, ketorolac, diclofenac, aceclofenac, nabumetone, piroxicam, meloxicam, tenoxicam, droxicam, lomoxicam, phenylbutazone, mefenamic acid, meclofenamic acid, flufenamic acid, tolfenamic acid, celecoxib, or combinations thereof.

In certain embodiments, the second therapeutic agent is a steroid. Examples of steroids considered for use in combination therapies of the invention include but are not limited to prednisone, prednisolone, methylprednisone, triacmcinolone, betamethasone, dexamethasone, and prodrugs thereof.

In certain embodiments, the second therapeutic agent is a tyrosine kinase inhibitor. Examples of tyrosine kinase inhibitors considered for use in combination therapies of the invention include but are not limited to inhibitors of the following kinases, including, among others: JAK, Syk, JNK/SAPK, MAPK, PI-3K, and/or Ripk2. In certain embodiments, the tyrosine kinase inhibitor is ruxolitinib, tofacitinib, oclactinib, filgotinib, ganotinib, lestaurtinib, momelotinib, pacritinib, upadacitinib, peficitinib, fedratinib, bentamapimod, D-JNKI-1 (XG-102, AM-111), ponatinib, WEHI-345, OD36, GSK583, idelalisib, copanlisib, taselisib, duvelisib, alpelisib, umbralisib, dactolisib, CUDC-907, entospletinib, fostamatinib, or combinations thereof.

In certain embodiments, the second therapeutic agent is a receptor kinase inhibitor, including among others, an inhibitor of EGFR or HER2. Examples of receptor kinase inhibitors considered for use in combination therapies of the invention include but are not limited to gefitinib, erlotinib, neratinib, lapatinib, cetuximab, panitumumab, vandetanib, necitumumab, osimertinib, trastuzumab, neratinib, lapatinib, pertuzumab, or combinations thereof.

In certain embodiments, the second therapeutic agent is a modulator of nuclear receptor family of transcription factors, including, among others, an inhibitor of PPAR, RXR, FXR, or LXR. In certain embodiments, the inhibitor is pioglitazone, bexarotene, obeticholic acid, ursodeoxycholic acid, fexaramine, hypocholamide, or combinations thereof.

In certain embodiments, the second therapeutic agent is an HSP90 inhibitor. Examples of HSP90 inhibitors considered for use in combination therapies of the invention include but are not limited to ganetespib, 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010, or combinations thereof.

In certain embodiments, the second therapeutic agent is an adenosine receptor 2A (A₂A) agonist. Examples of adenosine receptor agonists considered for use in combination therapies of the invention include but are not limited to those disclosed in U.S. Pat. No. 9,067,963, which is incorporated herein by reference. In certain embodiments, the adenosine receptor agonist is LNC-3050, LNC-3015, LNC-3047, LNC-3052, or combinations thereof.

In certain embodiments, the second therapeutic agent is selected from disease modifying antirheumatic drugs (DMARDS). Examples of DMARDS considered for use in combination therapies of the invention include but are not limited to tocilizumab, certolizumab, etanercept, adalimumab, anakinra, abatacept, infliximab, rituximab, golimumab, uteskinumab, or combinations thereof.

In certain embodiments, the second therapeutic agent is a phosphodiesterase (PDE) inhibitor. Examples of phosphodiesterase inhibitor considered for use in combination therapies of the invention include but are not limited to apremilast, crisaborole, piclimilast, drotaverine, ibudulast, roflumilast, sildenafil, tadalafil, vardenafil, or combinations thereof.

In certain embodiments, the second therapeutic agent is a neutrophil elastase inhibitor. Examples of neutrophil elastase inhibitors considered for use in combination therapies of the invention include but are not limited to sivelestat.

In certain embodiments, the second therapeutic agent is a modulator of Axl kinase. Examples of modulators of Axl kinase considered for use in combination therapies of the invention include but are not limited to bemcentinib (BGB324 or R428), TP-0903, LY2801653, amuvatinib (MP-470), bosutinib (SKI-606), MGCD 265, ASP2215, cabozantinib (XL184), foretinib (GSK1363089/XL880), and SGI-7079. In certain embodiments, the modulator of Axl kinase is a monoclonal antibody targeting AXL (e.g., YW327.6S2) or an AXL decoy receptor (e.g., GL2I.T), or glesatinib, merestinib, or a dual Flt3-Axl inhibitor such as gilteritinib.

In certain embodiments, the second therapeutic agent is a bispecific antibody, such as a bispecific antibody that binds to a tumor-specific antigen. Exemplary bispecific antibodies include but are not limited to Blincyto (blinatumomab), Kimmtrak (tebentafusp), Tecvayli (teclistamab), Lunsumio (mosunetuzumab), Epkinly (epcoritamab), and Columvi (glofitamab).

In certain embodiments, the second therapeutic agent is a chimeric antigen receptor (CAR) T-cell therapy. Exemplary CAR T-cell therapies include but are not limited to ABECMA® (idecabtagene vicleucel), BREYANZI® (lisocabtagene maraleucel), CARVYKTI™ (ciltacabtagene autoleucel), KYMRIAH™ (tisagenlecleucel), TECARTUS™ (brexucabtagene autoleucel), and YESCARTA™ (axicabtagene ciloleucel).

In certain embodiments, the additional therapeutic agent is an anti-cancer agent or chemo-therapeutic agent. Examples of anti-cancer agents considered for use in combination therapies of the invention include but are not limited erlotinib, bortezomib, fulvestrant, sunitib, imatinib mesylate, letrozole, finasunate, platins such as oxaliplatin, carboplatin, and cisplatin, finasunate, fluorouracil, rapamycin, leucovorin, lapatinib, lonafamib, sorafenib, gefitinib, camptothecin, topotecan, bryostatin, adezelesin, anthracyclin, carzelesin, bizelesin, dolastatin, auristatins, duocarmycin, eleutherobin, taxols such as paclitaxel or docetaxel, cyclophosphamide, doxorubicin, vincristine, prednisone or prednisolone, other alkylating agents such as mechlorethamine, chlorambucil, and ifosfamide, antimetabolites such as azathioprine or mercaptopurine, other microtubule inhibitors (vinca alkaloids like vincristine, vinblastine, vinorelbine, and vindesine, as well as taxanes), podophyllotoxins (etoposide, teniposide, etoposide phosphate, and epipodophyllotoxins), topoisomerase inhibitors, other cytotoxins such as actinomycin, daunorubicin, valrubicin, idarubicin, edrecolomab, epirubicin, bleomycin, plicamycin, mitomycin, as well as other anticancer antibodies (cetuximab, bevacizumab, ibritumomab, abagovomab, adecatumumab, afutuzumab, alacizumab, alemtuzumab, anatumomab, apolizumab, bavituximab, belimumab, bivatuzumab mertansine, blinatumomab, brentuximab vedotin, cantuzumab mertansine, catumazomab, cetuximab, citatuzumab bogatox, cixutumumab, clivatuzumab tetraxetan, conatumumab, dacetuzumab, daclizumab, detumomab, ecromeximab, edrecolomab, elotuzumab, epratuzumab, ertumaxomab, etaracizumab, farletuzumab, figitumumab, fresolimumab, galiximab, gembatumumab vedotin, gemtuzumab, ibritumomab tiuxetan, inotuzumab ozogamicin, intetumumab, ipilimumab, iratumumab, labetuzumab, lexatumumab, lintuzumab, lucatumumab, lumilisimab, mapatumumab, matuzumab, milatuzumab, mitumomab, nacolomab tafenatox, naptumomab estafenatox, necitumumab, nimotuzumab, ofatumumab, olaratumab, oportuzumab monatox, oregovomab, panitumumab, pemtumomab, pertuzumab, pintumomab, pritumumab, ramucirumab, rilotumumab, robatumumab, rituximab, sibrotuzumab, tacatuzumab tetraxetan, taplitumomab paptox, tenatumomab, ticilimumab, tigatuzumab, tositumomab or ¹³¹I-tositumomab, trastuzumab, tremelimumab, tuocotuzumab celmoleukin, veltuzumab, visilizumab, volocixumab, votumumab, zalutumumab, zanolimumab, IGN-101, MDX-010, ABX-EGR, EMD72000, ior-t1, MDX-220, MRA, H-11 scFv, huJ591, TriGem, TriAb, R3, MT-201, G-250, ACA-125, Onyvax-105, CD:-960, Cea-Vac, BrevaRex AR54, IMC-1C11, GlioMab-H, ING-1, anti-LCG MAbs, MT-103, KSB-303, Therex, KW2871, anti-HMI.24, Anti-PTHrP, 2C4 antibody, SGN-30, TRAIL-RI MAb, Prostate Cancer antibody, H22xKi-r, ABX-Mai, Imuteran, Monopharm-C), and antibody-drug conjugates comprising any of the above agents (especially auristatins MMAE and MMAF, maytansinoids like DM-1, calicheamycins, or various cytotoxins).

In certain embodiments, the additional therapeutic agent is selected from anastrozole (ARIMIDEX®), bicalutamide (CASODEX®), bleomycin sulfate (BLENOXANE®), busulfan (MYLERAN®), busulfan injection (BUSULFEX®), capecitabine (XELODAR), N4-pentoxycarbonyl-5-deoxy-5-fluorocytidine, carboplatin (PARAPLATIN®V), carmustine (BiCNU®), chlorambucil (LEUKERAN®), cisplatin (PLATINOL®), cladribine (LEUSTATIN®), cyclophosphamide (CYTOXAN® or NEOSAR®), cytarabine, cytosine arabinoside (CYTOSAR-U®), cytarabine liposome injection (DEPOCYT®), dacarbazine (DTIC-Dome®), dactinomycin (actinomycin D, COSMEGAN®), daunorubicin hydrochloride (CERUBIDINE®), daunorubicin citrate liposome injection (DAUNOXOME®), dexamethasone, docetaxel (TAXOTERE®), doxorubicin hydrochloride (ADRIAMYCIN®, RUBEX®), etoposide (VEPESID®), fludarabine phosphate (FLUDARA®), 5-fluorouracil (ADRUCIL®, EFUDEX®), flutamide (EULEXIN®), tezacitibine, gemcitabine (difluorodeoxycitidine), hydroxyurea (HYDREA®), idarubicin (IDAMYCIN®), ifosfamide (IFEX®), irinotecan (CAMPTOSAR®), L-asparaginase (ELSPAR®), leucovorin calcium, melphalan (ALKERAN®), 6-mercaptopurine (PURINETHOL®), methotrexate (FOLEX®), mitoxantrone (NOVANTRONE®), gemtuzumab ozogamicin (MYLOTARG™), paclitaxel (TAXOL®), nab-paclitaxel (ABRAXANE®), phoenix (Yttrium90/MX-DTPA), pentostatin, polifeprosan 20 with carmustine implant (GLIADEL®), tamoxifen citrate (NOLVADEX®), teniposide (VUMON®), 6-thioguanine, thiotepa, tirapazamine (TIRAZONE®), topotecan hydrochloride for injection (HYCAMPTIN®), vinblastine (VELBAN®), vincristine (ONCOVIN®), and vinorelbine (NAVELBINE®).

In certain embodiments, the additional therapeutic agent is capable of inhibiting BRAF, MEK, CDK4/6, SHP-2, HDAC, EGFR, MET, mTOR, PI3K or AKT, or a combination thereof. In a particular embodiment, the compounds of the present invention are combined with another therapeutic agent selected from vemurafinib, debrafinib, LGX818, trametinib, MEK162, LEE011, PD-0332991, panobinostat, verinostat, romidepsin, cetuximab, gefitinib, erlotinib, lapatinib, panitumumab, vandetanib, INC280, everolimus, simolimus, BMK120, BYL719 or CLR457, or a combination thereof.

In certain embodiments, the additional therapeutic agent is selected based on the disease or condition that is being treated. For example, in the treatment of melanoma, the additional therapeutic agent is selected from aldesleukin (e.g., PROLEUKIN®), dabrafenib (e.g., TAFINLAR®), dacarbazine, recombinant interferon alfa-2b (e.g., INTRON®A), ipilimumab, trametinib (e.g., MEKINIST®), peginterferon alfa-2b (e.g., PEGINTRON®, SYLATRON™), vemurafenib (e.g., ZELBORAF®)), and ipilimumab (e.g., YERVOY®).

For the treatment of ovarian cancer, the additional therapeutic agent is selected from doxorubicin hydrochloride (Adriamycin®), carboplatin (PARAPLATIN®), cyclophosphamide (CYTOXAN®, NEOSAR®), cisplatin (PLATINOLR, PLATINOL-AQ®), doxorubicin hydrochloride liposome (DOXIL, DOX-SL®, EVACETR, LIPODOX®), gemcitabine hydrochloride (GEMZAR®), topotecan hydrochloride (HYCAMTIN®), and paclitaxel (TAXOL®).

For the treatment of thyroid cancer, the additional therapeutic agent is selected from doxorubicin hydrochloride (Adriamycin®), cabozantinib-S-malate (COMETRIQ®), and vandetanib (CAPRELSA®).

For the treatment of colon cancer, the additional therapeutic agent is selected from fluorouracil (e.g., ADRUCIL®, EFUDEX®, FLUOROPLEX®), bevacizumab (AVASTIN®), irinotecan hydrochloride (CAMPTOSTAR®), capecitabine (XELODA®), cetuximab (ERBITUX®), oxaliplatin (ELOXATIN®), leucovorin calcium (WELLCOVORIN®), regorafenib (STIVARGA®), panitumumab (VECTIBIX®), and ziv-aflibercept (ZALTRAP®).

For the treatment of lung cancer, the additional therapeutic agent is selected from methotrexate, methotrexate LPF (e.g., FOLEX®, FOLEX PFS®, Abitrexate®, MEXATE®, MEXATE-AQ®), paclitaxel (TAXOL®), paclitaxel albumin-stabilized nanoparticle formulation (ABRAXANE®), afatinib dimaleate (GILOTRIF®), pemetrexed disodium (ALIMTA®), bevacizumab (AVASTIN®), carboplatin (PARAPLATIN®), cisplatin (PLATINOL®, PLATINOL-AQ®), crizotinib (XALKORI®), erlotinib hydrochloride (TARCEVA®), gefitinib (IRESSA®), and gemcitabine hydrochloride (GEMZAR®).

For the treatment of pancreatic cancer, the other therapeutic agent may be selected from fluorouracil (ADRUCIL®), EFUDEX®, FLUOROPLEX®), erlotinib hydrochloride (TARCEVA®), gemcitabine hydrochloride (GEMZAR®), and mitomycin or mitomycin C (MITOZYTREX™, MUTAMYCIN®).

For the treatment of cervical cancer, the additional therapeutic agent is selected from bleomycin (BLENOXANE®), cisplatin (PLATINOL®, PLATINOL-AQ®) and topotecan hydrochloride (HYCAMTIN®).

For the treatment of head and neck cancer, the additional therapeutic agent is selected from methotrexate, methotrexate LPF (e.g., FOLEX®, FOLEX PFS®, Abitrexate®, MEXATE®, MEXATE-AQ®), fluorouracil (ADRUCIL®, EFUDEX®, FLUOROPLEX®), bleomycin (BLENOXANE®), cetuximab (ERBITUX®), cisplatin (PLATINOL®, PLATINOL-AQ®) and docetaxel (TAXOTERE®).

For the treatment of leukemia, including chronic myelomonocytic leukemia (CMML), the additional therapeutic agent is selected from bosutinib (BOSULIF®), cyclophosphamide (CYTOXAN®, NEOSAR®), cytarabine (CYTOSAR-U®, TARABINE PFS®), dasatinib (SPRYCEL®), imatinib mesylate (GLEEVEC®), ponatinib (ICLUSIG®), nilotinib (TASIGNA®) and omacetaxine mepesuccinate (SYNRIBO®).

In some instances, patients may experience allergic reactions to the compounds of the present invention and/or other anti-cancer agent(s) during or after administration. Therefore, anti-allergic agents may be administered to minimize the risk of an allergic reaction. Suitable anti-allergic agents include corticosteroids, such as dexamethasone (e.g., DECADRON®), beclomethasone (e.g., BECLOVENT®), hydrocortisone (also known as cortisone, hydrocortisone sodium succinate, hydrocortisone sodium phosphate; e.g., ALA-CORT®, hydrocortisone phosphate, Solu-CORTEF®, HYDROCORT Acetate® and LANACORT®), prednisolone (e.g., DELTA-Cortel®, ORAPRED®, PEDIAPRED® and PRELONE®), prednisone (e.g., DELTASONE®, LIQUID RED®, METICORTEN® and ORASONE®), methylprednisolone (also known as 6-methylprednisolone, methylprednisolone acetate, methylprednisolone sodium succinate; e.g., DURALONE®, MEDRALONE®, MEDROL®, M-PREDNISOL® and SOLU-MEDROL®); antihistamines, such as diphenhydramine (e.g., BENADRYL®), hydroxyzine, and cyproheptadine; and bronchodilators, such as the beta-adrenergic receptor agonists, albuterol (e.g., PROVENTIL®), and terbutaline (BRETHINE®).

In other instances, patients may experience nausea during and after administration of the compound of the present invention and/or other anti-cancer agent(s). Therefore, anti-emetics may be administered in preventing nausea (upper stomach) and vomiting. Suitable anti-emetics include aprepitant (EMEND®), ondansetron (ZOFRAN®), granisetron HCl (KYTRIL®), lorazepam (ATIVAN®, dexamethasone (DECADRON®), prochlorperazine (COMPAZINE®), casopitant (REZONIC® and Zunrisa®), and combinations thereof.

In yet other instances, medication to alleviate the pain experienced during the treatment period is prescribed to make the patient more comfortable. Common over-the-counter analgesics, such TYLENOL®, are often used. Opioid analgesic drugs such as hydrocodone/paracetamol or hydrocodone/acetaminophen (e.g., VICODIN®), morphine (e.g., ASTRAMORPH® or AVINZA®), oxycodone (e.g., OXYCONTIN® or PERCOCET®), oxymorphone hydrochloride (OPANA®), and fentanyl (e.g., DURAGESIC®) are also useful for moderate or severe pain.

Furthermore, cytoprotective agents (such as neuroprotectants, free-radical scavengers, cardioprotectors, anthracycline extravasation neutralizers, nutrients and the like) may be used as an adjunct therapy to protect normal cells from treatment toxicity and to limit organ toxicities. Suitable cytoprotective agents include amifostine (ETHYOL®), glutamine, dimesna (TAVOCEPT®), mesna (MESNEX®), dexrazoxane (ZINECARD® or TOTECT®), xaliproden (XAPRILA®), and leucovorin (also known as calcium leucovorin, citrovorum factor and folinic acid).

In yet another aspect, a compound of the present invention may be used in combination with known therapeutic processes, for example, with the administration of hormones or in radiation therapy. In certain instances, a compound of the present invention may be used as a radiosensitizer, especially for the treatment of tumors which exhibit poor sensitivity to radiotherapy.

The doses and dosage regimen of the active ingredients used in the combination therapy may be determined by an attending clinician. In certain embodiments, the compound described herein (e.g., a compound of Formula I, Ia, Ib, Ic, Id, Ie, If, Ig, Ih, Ii, Ij, Ik, Ii, Im, In, Io, Ip, Iq, Ir, Is, It, Iu, Iv, Iw, Ix, Iy, Iz, I-1, Ia-1, Tb-1, Ic-1, Id-1, Ie-1, If-1, Ig-1, Ih-1, Ii-1, Ij-1, Ik-1, Il-1, Im-1, In-1, Io-1, Ip-1, Iq-1, Ir-1, Is-1, It-1, Iu-1, Iv-1, Iw-1, or Ix-1, or other compounds in Section I) and the additional therapeutic agent(s) are administered in doses commonly employed when such agents are used as monotherapy for treating the disease or condition. In other embodiments, the compound described herein (e.g., a compound of Formula I, Ia, Ib, Ic, Id, Ie, If, Ig, Ih, Ii, Ij, Ik, Ii, Im, In, Io, Ip, Iq, Ir, Is, It, Iu, Iv, Iw, Ix, Iy, Iz, I-1, Ia-1, Tb-1, Ic-1, Id-1, Te-1, If-1, Ig-1, Ih-1, Ii-1, Ij-1, Ik-1, Il-1, Im-1, In-1, Io-1, Ip-1, Iq-1, Ir-1, Is-1, It-1, Iu-1, Iv-1, Iw-1, or Ix-1, or other compounds in Section I) and the additional therapeutic agent(s) are administered in doses lower than the doses commonly employed when such agents are used as monotherapy for treating the disease or condition. In certain embodiments, the compound described herein (e.g., a compound of Formula I, Ia, Tb, Ic, Id, Ie, If, Ig, Ih, Ii, Ij, Ik, Il, Im, In, Io, Ip, Iq, Ir, Is, It, Iu, Iv, Iw, Ix, Iy, Iz, I-1, Ia-1. Tb-1, Ic-1, Id-1, Te-1, If-1, Ig-1, Ih-1, Ii-1, Ij-1, Ik-1, Il-1, Im-1, In-1, Io-1, Ip-1, Iq-1, Ir-1, Is-1, It-1, Iu-1, Iv-1, Iw-1, or Ix-1, or other compounds in Section I) and the additional therapeutic agent(s) are present in the same composition, which is suitable for oral administration.

In certain embodiments, the compound described herein (e.g., a compound of Formula I, Ta, Tb, Ic, Id, Ie, If, Ig, Ih, Ii, Ij, Ik, Il, Im, In, Io, Ip, Iq, Ir, Is, It, Iu, Iv, Tw, Ix, Iy, Iz, I-1, Ta-1, Tb-1, Ic-1, Id-1, Te-1, If-1, Ig-1, Ih-1, Ii-1, Ij-1, Ik-1, I-1, Im-1, In-1, Io-1, Ip-1, Iq-1, Ir-1, Is-1, It-1, Iu-1, Iv-1, Iw-1, or Ix-1, or other compounds in Section I) and the additional therapeutic agent(s) may act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

Another aspect of this invention is a kit comprising a therapeutically effective amount of a compound described herein (e.g., a compound of Formula I, Ia, Ib, Ic, Id, Ie, If, Ig, Ih, Ii, Ij, Ik, Il, Im, In, Io, Ip, Iq, Ir, Is, It, Iu, Iv, Iw, Ix, Iy, Iz, I-1, Ia-1. Tb-1, Ic-1, Id-1, Te-1, If-1, Ig-1, Ih-1, Ii-1, Ij-1, Ik-1, Il-1, Im-1, In-1, Io-1, Ip-1, Iq-1, Ir-1, Is-1, It-1, Iu-1, Iv-1, Iw-1, or Ix-1, or other compounds in Section I), a pharmaceutically acceptable carrier, vehicle or diluent, and optionally at least one additional therapeutic agent listed above. In certain embodiments, the kit further comprises instructions, such as instructions for treating a disease described herein.

### IV. Pharmaceutical Compositions and Dosing Considerations

As indicated above, the invention provides pharmaceutical compositions, which comprise a therapeutically-effective amount of one or more of the compounds described above, formulated together with one or more pharmaceutically acceptable carriers (additives) and/or diluents. The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. In certain embodiments, the invention provides a pharmaceutical composition comprising a compound described herein (e.g., a compound of Formula I, Ia, Ib, Ic, Id, Ie, If, Ig, Ih, Ii, Ij, Ik, Il, Im, In, Io, Ip, Iq, Ir, Is, It, Iu, Iv, Iw, Ix, Iy, Iz, I-1, Ia-1, Tb-1, Ic-1, Id-1, Ie-1, If-1, Ig-1, Ih-1, Ii-1, Ij-1, Ik-1, Il-1, Im-1, In-1, Io-1, Ip-1, Iq-1, Ir-1, Is-1, It-1, Iu-1, Iv-1, Iw-1, or Ix-1, or other compounds in Section I) and a pharmaceutically acceptable carrier.

The phrase “therapeutically effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (e.g., capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

The invention further provides a unit dosage form (such as a tablet or capsule) comprising a imidazopyrimidine compound or related compound described herein in a therapeutically effective amount for the treatment of a disease or condition described herein.

### IV. Medical Kits

Another aspect of the invention provides a medical kit comprising, for example, (i) a compound described herein, and (ii) instructions for use according to a method described herein.

### V. Enumerated Embodiments

The following exemplary embodiments are provided:

Embodiment 1 provides a compound of formula I-1:

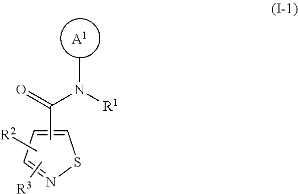

or a pharmaceutically acceptable salt thereof; wherein:
A¹ is phenyl, a 6 membered heteroaryl containing 1 or 2 nitrogen atoms, a 9-10 membered bicyclic heteroaryl containing 1, 2, or 3 heteroatoms independently selected from nitrogen and oxygen,

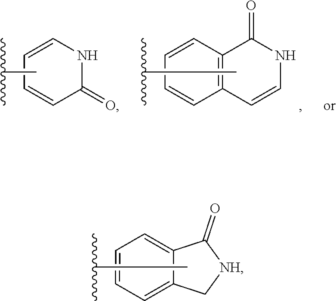

wherein the phenyl, heteroaryl,

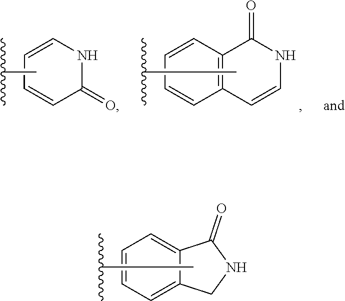

are substituted with m occurrences of R⁵ and n occurrences of R⁶;
A² represents independently for each occurrence a 4-6 membered saturated heterocyclyl containing 1 or 2 heteroatoms independently selected from oxygen, nitrogen, and sulfur, wherein the heterocyclyl is substituted with y occurrences of R⁷;
R¹ is hydrogen or C₁₋₄ alkyl;
R² is C-6 haloalkyl, C-6 alkyl, C₃₋₇ cycloalkyl, C₁₋₆ alkoxyl, cyano, C₂₋₄ alkenyl, halogen, hydrogen, oxetanyl, tetrahydrofuranyl, or —N(R¹²)(R¹³), wherein the oxetanyl and tetrahydrofuranyl are substituted with 0 or 1 hydroxyl groups;
R³ is one of the following:
   (a) phenyl, a 5-6 membered heteroaryl containing 1, 2, or 3 heteroatoms independently selected from oxygen, nitrogen, and sulfur, a 9-10 membered bicyclic heteroaryl containing 1, 2, or 3 heteroatoms independently selected from oxygen, nitrogen, and sulfur, a 3-10 membered monocyclic or bicyclic saturated or partially unsaturated heterocyclyl containing 1, 2, or 3 heteroatoms independently selected from oxygen, nitrogen, and sulfur,

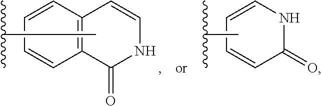

wherein the phenyl, heteroaryl, heterocyclyl,

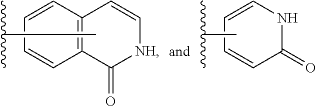

are substituted with t occurrences of R⁴; or
(b) C₂₋₆ alkyl, hydroxyl, or —N(R⁹)(R¹⁰);
R⁴ represents independently for each occurrence halo, hydroxyl, C₁₋₆ alkyl, C₁₋₆ haloalkyl, C₁₋₆hydroxyalkyl, C₁₋₆ alkoxyl, C₃₋₇ cycloalkyl, —C(O)R¹¹, —C(O)N(R⁹)(R¹⁰), or —N(R⁹)C(O)R¹¹;
R⁵ is a 5-6 membered heteroaryl containing 1, 2, or 3 heteroatoms independently selected from oxygen, nitrogen, and sulfur, or a 3-7 membered saturated heterocyclyl containing 1, 2, or 3 heteroatoms independently selected from oxygen, nitrogen, and sulfur, wherein the heteroaryl and heterocyclyl are substituted with q occurrences of R⁷;
R⁶ represents independently for each occurrence halo, hydroxyl, C₁₋₆ alkyl, C₁₋₆ haloalkyl, C₁₋₆hydroxyalkyl, C₁₋₆ alkoxyl, C₃₋₇ cycloalkyl, cyano, —O—C₃₋₇ cycloalkyl, —N(R⁹)(R¹⁰), —(C₀₋₄ alkylene)-C(O)R^{•}, —C(O)N(R⁹)(R¹⁰), or —N(R⁹)C(O)R¹¹;
R⁷ represents independently for each occurrence halo, hydroxyl, C₁₋₆ alkyl, C₁₋₆ haloalkyl, C₁₋₆ alkoxyl, or C₃₋₇ cycloalkyl;
R⁸ is —OH, —O—(C₁₋₆ alkyl), —O—C₃₋₇ cycloalkyl, or A²;
R⁹ and R¹⁰ are independently hydrogen, C₁₋₆ alkyl, C₃₋₇ cycloalkyl, C₂₋₄ hydroxyalkyl, or —(C₂₋₆ alkylene)-(C₁₋₆ alkoxyl), or R⁹ and R¹⁰ are taken together with the nitrogen atom to which they are attached to form a 3-7 membered heterocyclic ring containing 1 nitrogen atom, wherein the heterocyclic ring is substituted with 0 or 1 groups independently selected from hydroxyl, halo, and C₁₋₆ alkyl;
R¹¹ represents independently for each occurrence C₁₋₆ alkyl or (Co-s alkylene)-C₃₋₇ cycloalkyl;
R¹² and R¹³ are independently hydrogen, C₁₋₆ alkyl, or C₃₋₅ cycloalkyl, or R¹² and R¹³ are taken together with the nitrogen atom to which they are attached to form a 3-7 membered heterocyclic ring containing 1 nitrogen atom;
m is 0 or 1; and
n, q, t, and y are independently 0, 1, or 2;
provided that when R² is hydrogen and m is 1, then R⁵ is 1,2,3-triazolyl substituted with q occurrences of R⁷.

Embodiment 2 provides the compound of embodiment 1, wherein the compound is a compound of Formula I-1.

Embodiment 3 provides the compound of embodiment 1 or 2, wherein R² is C₁₋₆ haloalkyl.

Embodiment 4 provides the compound of embodiment 1 or 2, wherein R² is —CF₃.

Embodiment 5 provides the compound of embodiment 1 or 2, wherein R² is C₃₋₇ cycloalkyl.

Embodiment 6 provides the compound of embodiment 1 or 2, wherein R² is cyclopropyl.

Embodiment 7 provides the compound of embodiment 1 or 2, wherein R² is C₁₋₆ alkyl or C₂₋₄ alkenyl.

Embodiment 8 provides the compound of embodiment 1 or 2, wherein R² is halogen, C₁-6 alkoxyl, or cyano.

Embodiment 9 provides the compound of any one of embodiments 1-8, wherein the compound is a compound of Formula Ia-1 or a pharmaceutically acceptable salt thereof:

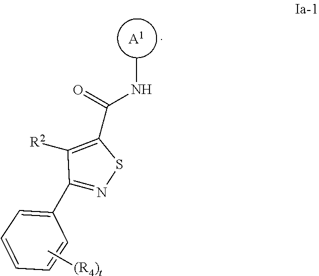

Embodiment 10 provides the compound of any one of embodiments 1-8, wherein the compound is a compound of Formula Ib-1 or Ic-1 or a pharmaceutically acceptable salt thereof:

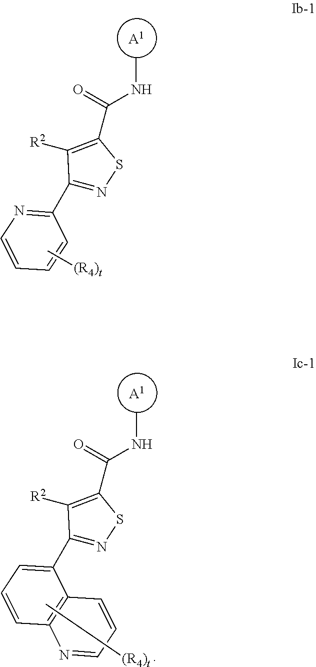

Embodiment 11 provides the compound of any one of embodiments 1-10, wherein A¹ is phenyl substituted with m occurrences of R⁵ and n occurrences of R⁶.

Embodiment 12 provides the compound of any one of embodiments 1-10, wherein A¹ is a 6 membered heteroaryl containing 1 or 2 nitrogen atoms, wherein the heteroaryl is substituted with m occurrences of R⁵ and n occurrences of R⁶.

Embodiment 13 provides the compound of any one of embodiments 1-10, wherein A¹ is pyridinyl substituted with m occurrences of R⁵ and n occurrences of R⁶.

Embodiment 14 provides the compound of any one of embodiments 1-10, wherein A¹ is a 9-10 membered bicyclic heteroaryl containing 1, 2, or 3 heteroatoms independently selected from nitrogen and oxygen, wherein the heteroaryl is substituted with m occurrences of R⁵ and n occurrences of R⁶.

Embodiment 15 provides the compound of any one of embodiments 1-10, wherein A¹ is

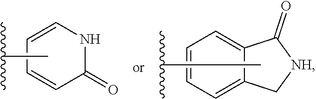

each of which is substituted with m occurrences of R⁵ and n occurrences of R⁶.

Embodiment 16 provides the compound of any one of embodiments 1-15, wherein n is 1.

Embodiment 17 provides the compound of any one of embodiments 1-15, wherein n is 0.

Embodiment 18 provides the compound of any one of embodiments 1-17, wherein m is 1.

Embodiment 19 provides the compound of any one of embodiments 1-8, wherein the compound is a compound of Formula Id-1 or a pharmaceutically acceptable salt thereof:

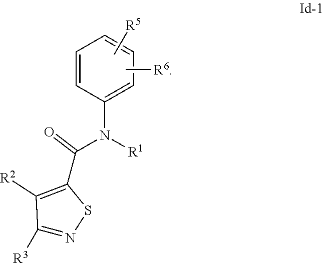

Embodiment 20 provides the compound of any one of embodiments 1-8, wherein the compound is a compound of Formula Ie-1 or a pharmaceutically acceptable salt thereof:

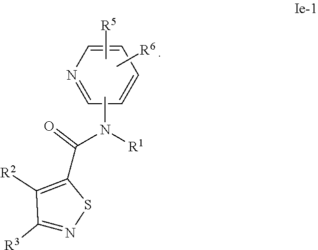

Embodiment 21 provides the compound of any one of embodiments 1-8, wherein the compound is a compound of Formula If-1 or a pharmaceutically acceptable salt thereof:

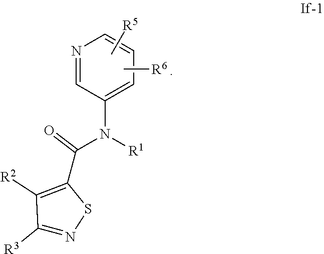

Embodiment 22 provides the compound of any one of embodiments 1-6 or 19-21, wherein R³ is phenyl substituted with t occurrences of R⁴.

Embodiment 23 provides the compound of any one of embodiments 1-6 or 19-21, wherein R³ is a 5-6 membered heteroaryl containing 1, 2, or 3 heteroatoms independently selected from oxygen, nitrogen, and sulfur, wherein the heteroaryl is substituted with t occurrences of R⁴.

Embodiment 24 provides the compound of any one of embodiments 1-6 or 19-21, wherein R³ is a 9-10 membered bicyclic heteroaryl containing 1, 2, or 3 heteroatoms independently selected from oxygen, nitrogen, and sulfur, wherein the heteroaryl is substituted with t occurrences of R⁴.

Embodiment 25 provides the compound of any one of embodiments 1-6 or 19-21, wherein R³ is a 3-10 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁴.

Embodiment 26 provides the compound of any one of embodiments 1-6 or 19-21, wherein R³ is tetrahydropyranyl, morpholinyl, piperidinyl, or piperazinyl, each of which is substituted with t occurrences of R⁴.

Embodiment 27 provides the compound of any one of embodiments 1-6 or 19-21, wherein R³ is

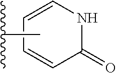

substituted with t occurrences of R⁴.

Embodiment 28 provides the compound of any one of embodiments 1-6 or 19-21, wherein R³ is C₃₋₄ alkyl or hydroxyl.

Embodiment 29 provides the compound of any one of embodiments 1-6 or 19-28, wherein R¹ is hydrogen.

Embodiment 30 provides the compound of any one of embodiments 1-6, wherein the compound is a compound of Formula Ig-1 or Ih-1 or a pharmaceutically acceptable salt thereof:

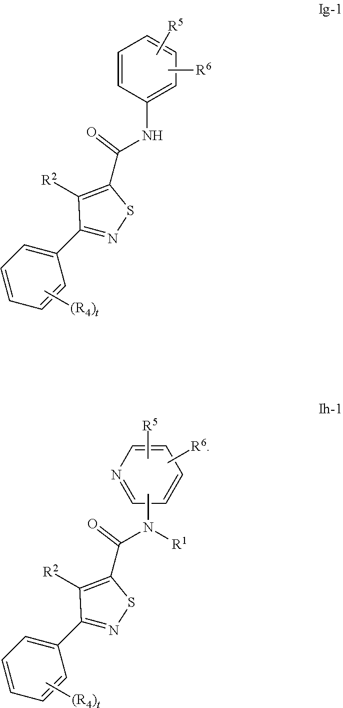

Embodiment 31 provides the compound of any one of embodiments 1-6, wherein the compound is a compound of Formula Ii-1, Ij-1, Ik-1, or Il-1 or a pharmaceutically acceptable salt thereof:

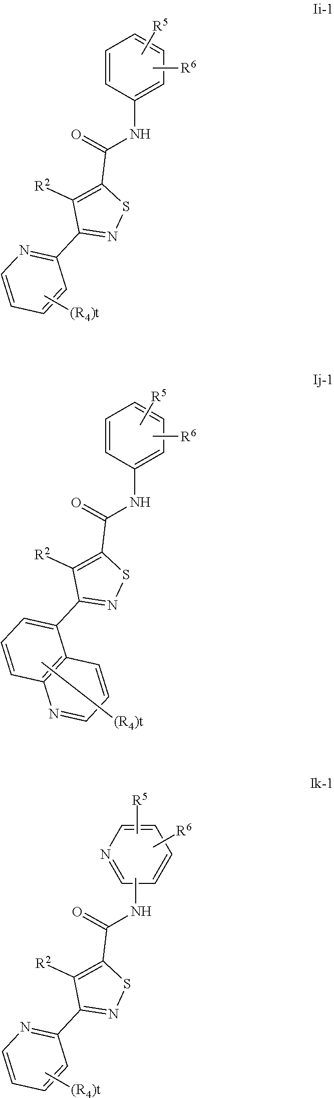

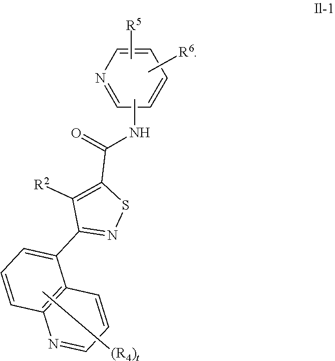

Embodiment 32 provides the compound of any one of embodiments 1-6, wherein the compound is a compound of Formula Im-1 or In-1 or a pharmaceutically acceptable salt thereof:

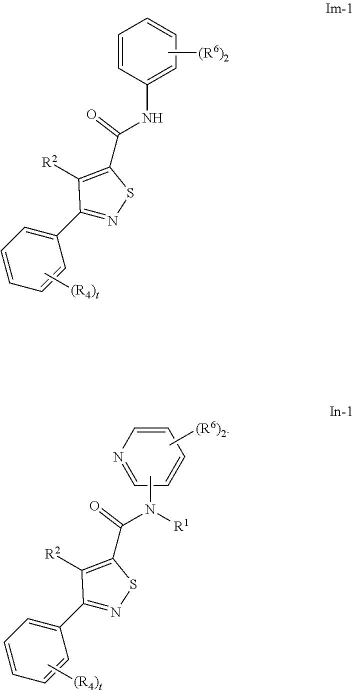

Embodiment 33 provides the compound of any one of embodiments 1-6, wherein the compound is a compound of Formula Io-1, Ip-1, Iq-1, or Ir-1 or a pharmaceutically acceptable salt thereof

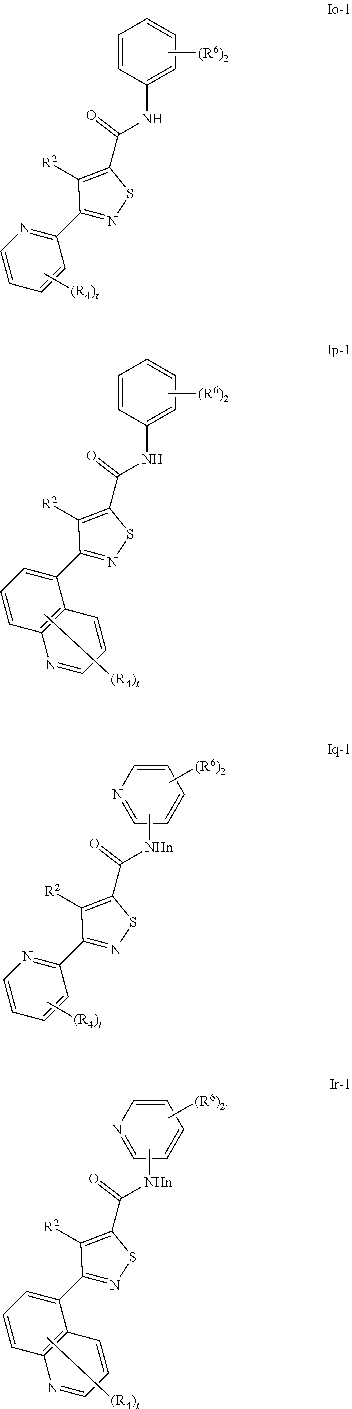

Embodiment 34 provides the compound of any one of embodiments 1-6, wherein the compound is a compound of Formula Is-1 or It-1 or a pharmaceutically acceptable salt thereof:

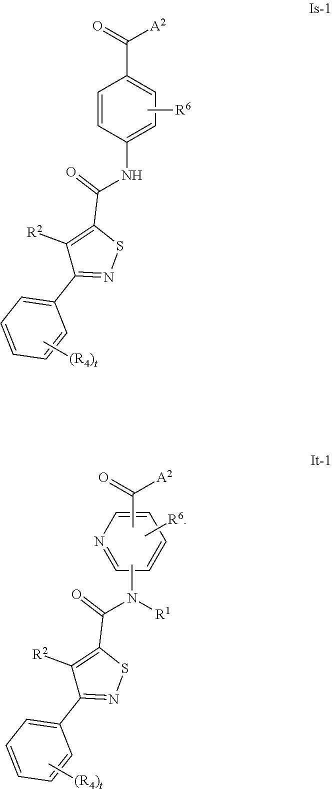

Embodiment 35 provides the compound of any one of embodiments 1-6, wherein the compound is a compound of Formula Iu-1, Iv-1, Iw-1, or Ix-1 or a pharmaceutically acceptable salt thereof:

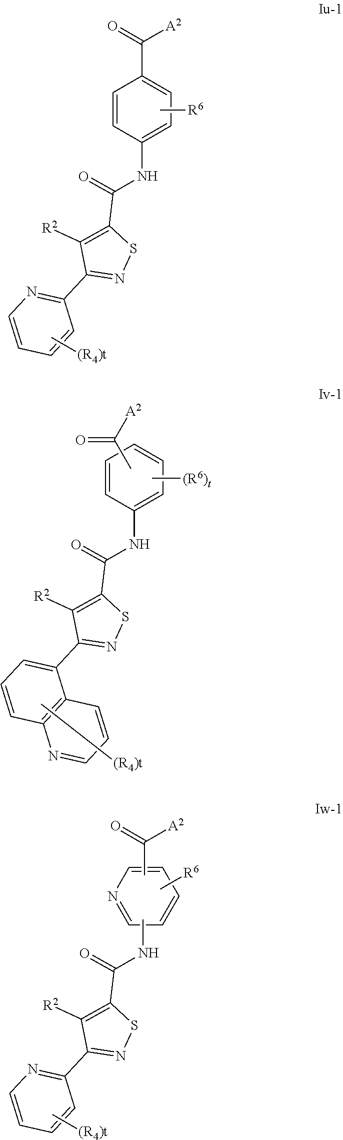

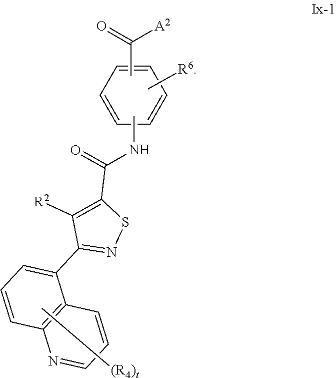

Embodiment 36 provides the compound of any one of embodiments 1-31, wherein R⁵ is a 5 membered heteroaryl containing 1, 2, or 3 heteroatoms independently selected from oxygen, nitrogen, and sulfur, wherein the heteroaryl is substituted with q occurrences of R.

Embodiment 37 provides the compound of any one of embodiments 1-31, wherein R⁵ is a 1,2,3-triazolyl, pyrazolyl, oxazolyl, imidazolyl, isoxazolyl, pyrrolyl, or furanyl, each of which substituted with q occurrences of R².

Embodiment 38 provides the compound of any one of embodiments 1-31, wherein R⁵ is 1,2,3-triazolyl substituted with q occurrences of R⁷.

Embodiment 39 provides the compound of any one of embodiments 1-31 or 36-38, wherein q is 0.

Embodiment 40 provides the compound of any one of embodiments 1-39, wherein R⁶ represents independently for each occurrence halo, C₁₋₆ alkyl, C₁₋₆ haloalkyl, cyano, or —(C₀₋₄ alkylene)-C(O)R⁸.

Embodiment 41 provides the compound of any one of embodiments 1-39, wherein R⁶ represents independently for each occurrence halo, cyano, or —(C₀₋₄ alkylene)-C(O)R⁸.

Embodiment 42 provides the compound of any one of embodiments 1-39, wherein R⁶ is chloro.

Embodiment 43 provides the compound of any one of embodiments 1-39, wherein R⁶ is C₁₋₆ haloalkyl.

Embodiment 44 provides the compound of any one of embodiments 1-39, wherein R⁶ is —CF₃.

Embodiment 45 provides the compound of any one of embodiments 1-39, wherein R⁶ is cyano.

Embodiment 46 provides the compound of any one of embodiments 1-45, wherein A² is azetidinyl substituted by hydroxyl.

Embodiment 47 provides the compound of any one of embodiments 1-46, wherein R⁴ represents independently for each occurrence halo, hydroxyl, C₁₋₆ alkyl, C₁₋₆ haloalkyl, or C₃₋₇ cycloalkyl.

Embodiment 48 provides the compound of any one of embodiments 1-46, wherein R⁴ represents independently for each occurrence halo or C₁₋₆ alkyl.

Embodiment 49 provides the compound of any one of embodiments 1-48, wherein t is 1.

Embodiment 50 provides the compound of any one of embodiments 1-47, wherein t is 0.

Embodiment 51 provides a compound in Table 1, or a pharmaceutically acceptable salt thereof.

Embodiment 52 provides a pharmaceutical composition comprising a compound of any one of embodiments 1-51 and a pharmaceutically acceptable carrier.

Embodiment 53 provides a method for treating a disease or condition mediated by MALT1, comprising administering to a subject in need thereof a therapeutically effective amount of a compound of any one of embodiments 1-51 to treat the disease or condition.

Embodiment 54 provides the method of embodiment 53, wherein said disease or condition mediated by MALT1 is a proliferative disorder.

Embodiment 55 provides the method of embodiment 53, wherein said disease or condition mediated by MALT1 is an inflammatory disorder.

Embodiment 56 provides the method of embodiment 53, wherein said disease or condition mediated by MALT1 is an autoimmune disorder.

Embodiment 57 provides the method of embodiment 53, wherein said disease or condition mediated by MALT1 is selected from cancer, neoplasia, chronic inflammatory disorder, acute inflammatory disorder, auto-inflammatory disorder, autoimmune disorder, fibrotic disorder, metabolic disorder, cardiovascular disorder, cerebrovascular disorder, myeloid cell-driven hyper-inflammatory response in COVID-19 infection, and a combination thereof.

Embodiment 58 provides the method of embodiment 53, wherein said disease or condition mediated by MALT1 is cancer.

Embodiment 59 provides the method of embodiment 58, wherein the cancer is lung cancer, pancreatic cancer, colorectal cancer, breast cancer, cervical cancer, prostate cancer, gastric cancer, skin cancer, liver cancer, bile duct cancer, nervous system cancer, a lymphoma, or a leukemia.

Embodiment 60 provides the method of embodiment 53, wherein said disease or condition mediated by MALT1 is Hodgkin's lymphoma, non-Hodgkin's lymphoma, Burkitt's lymphoma, diffuse large B-cell lymphoma (DLBCL), MALT lymphoma, germinal center B-cell-like diffuse large B-cell lymphoma (GCB-DLBCL), primary mediastinal B-cell lymphoma (PMBL), or activated B-cell-like diffuse large B-cell lymphoma (ABC-DLBCL).

Embodiment 61 provides the method of embodiment 53, wherein said disease or condition mediated by MALT1 is multiple sclerosis, ankylosing spondylitis, arthritis, osteoarthritis, juvenile arthritis, reactive arthritis, rheumatoid arthritis, psoriatic arthritis, acquired immunodeficiency syndrome (AIDS), Coeliac disease, psoriasis, chronic graft-versus-host disease, acute graft-versus-host disease, Crohn's disease, inflammatory bowel disease, multiple sclerosis, systemic lupus erythematosus, Celiac Sprue, idiopathic thrombocytopenic thrombotic purpura, myasthenia gravis, Sjogren's syndrome, scleroderma, ulcerative colitis, asthma, uveitis, rosacea, dermatitis, alopecia areata, vitiligo, arthritis, Type 1 diabetes, lupus erythematosus, systemic lupus erythematosus, Hashimoto's thyroiditis, myasthenia gravis, nephrotic syndrome, eosinophilia fasciitis, hyper IgE syndrome, lepromatous leprosy, sezary syndrome, idiopathic thrombocytopenia purpura, restenosis following angioplasty, a tumor, or artherosclerosis.

Embodiment 62 provides the method of embodiment 53, wherein said disease or condition mediated by MALT1 is allergic rhinitis, nasal inflammation, asthma, chronic obstructive pulmonary disease (COPD), bronchitis, emphysema, chronic eosinophilic pneumonia, adult respiratory distress syndrome, sinusitis, allergic conjunctivitis, idiopathic pulmonary fibrosis, atopic dermatitis, asthma, allergic rhinitis, arthritis, inflammatory bowel disease, Crohn's disease, ulcerative colitis, multiple sclerosis, endometriosis, eczema, psoriasis, rosacea, or lupus erythematosus.

Embodiment 63 provides the method of any one of embodiments 53-62, wherein the subject is a human.

Embodiment 64 provides a method of inhibiting the activity of MALT1, comprising contacting a MALT1 with an effective amount of a compound of any one of embodiments 1-51 to inhibit the activity of said MALT1.

### Examples

The invention now being generally described, will be more readily understood by reference to the following examples, which are included merely for purposes of illustrating certain aspects and embodiments of the present invention, and are not intended to limit the invention.

### General Procedures

The following general procedures were used in certain instances. Examples below may refer to one of the following general procedures. NMR chemical shift data are presented in ppm values. The term DCM refers to dichloromethane.

### General Procedure A: For Amide Bond Couplings

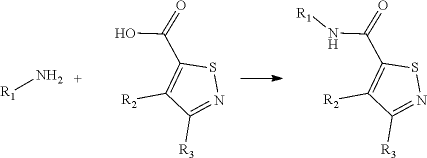

To a stirred solution of a 1,2-thiazole-5-carboxylic acid (1 eq.) and an amine (2 eq.) in pyridine is added POCl₃ (1.1 to 2 eq.) at 0° C. under nitrogen. The resulting mixture is stirred for 2 h at room temperature under nitrogen atmosphere. The resulting mixture is carefully quenched with water (20 mL) and extracted with EtOAc. The combined organic layers are washed with brine and dried over anhydrous Na₂SO₄. After filtration, the filtrate is concentrated under reduced pressure, and the crude product is purified by preparative HPLC.

### General Procedure B: For Saponification of Methyl 1,2-Thiazole-5-Carboxylate Esters

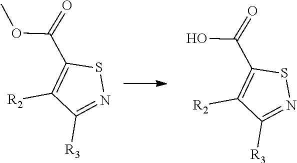

A solution of methyl 1,2-thiazole-5-carboxylate (1 eq.) and LiOH (3 eq.) in THF/H₂O (4:1) is stirred for 2 h at room temperature under nitrogen. The resulting mixture is diluted with water. The pH is adjusted to ˜6 with 2 N HCl, and the mixture is extracted with EtOAc. The combined organic layers are washed with water, dried over MgSO₄ or Na₂SO₄, and concentrated under reduced pressure. The material is generally used without further purification, unless otherwise specified.

### General Procedure C: For Further Amide Bond Couplings

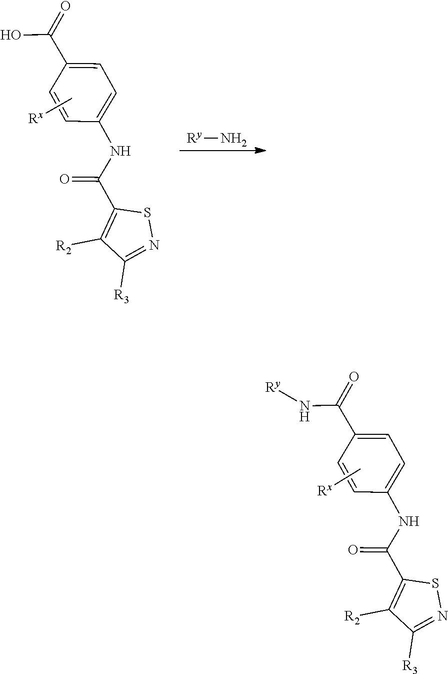

To a stirred solution of 4-(isothiazole-5-carboxamido)benzoic acid (1.0 eq) and HATU (1.5 eq) in DMF is added an amine (1.1 eq.) at room temperature, and the resulting mixture is stirred for 2 h at room temperature under nitrogen. The mixture is then diluted with water, and extracted with DCM. The combined organic layers are washed with brine, dried over anhydrous Na₂SO₄, and concentrated under reduced pressure. The crude product is purified by Prep-HPLC to afford the desired compound.

### General Procedure D: For Further Amide Bond Couplings

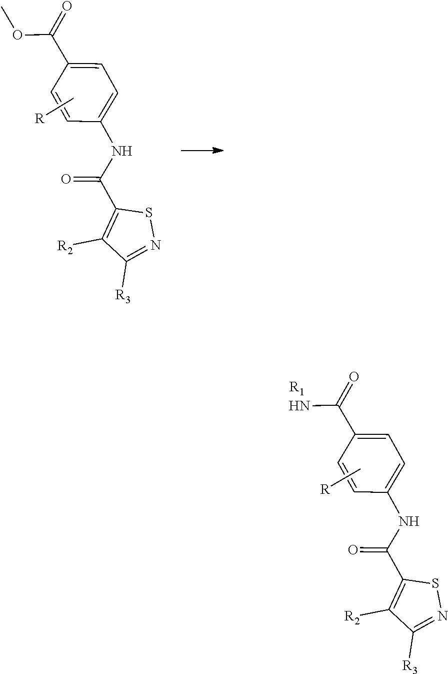

To a stirred solution of 4-(isothiazole-5-carboxamido)benzoic ester (1.0 eq.) and an amine (1.1 eq.) in THF is added t-BuOK in THF (1 M) (3 eq.) at room temperature. The resulting mixture is stirred for 0.5 h at room temperature under nitrogen. The reaction is quenched with sat. NH₄Cl (aq.) at 0° C. and extracted with ethyl acetate (EA). The combined organic layers are washed with brine, dried over anhydrous Na₂SO₄, and concentrated under reduced pressure. The crude product is purified by Prep-HPLC to afford the title compound.

### General Procedure E: For Removal of Tert-Butyldimethylsilyl Group

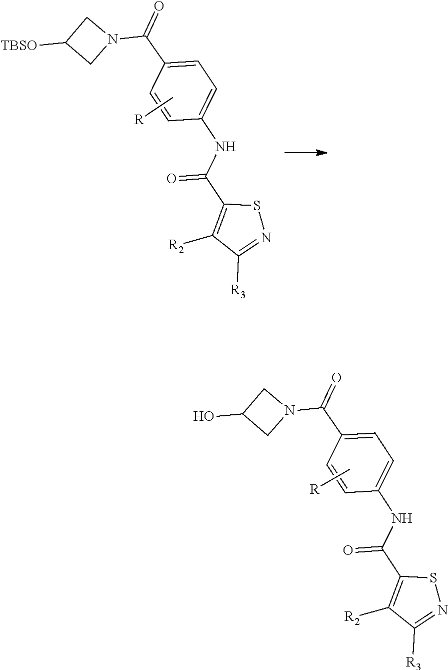

A mixture of N-(4-(3-((tert-butyldimethylsilyl)oxy)azetidine-1-carbonyl)-3-phenyl)-isothiazole-5-carboxamide (1.0 eq.) and CsF (2.0 eq.) in MeOH is stirred for 2 h at room temperature under nitrogen. After filtration, the filtrate is concentrated under reduced pressure. The crude product is purified by Prep-HPLC to afford the title compound.

### General Procedure F: For Further Carbon Bond Couplings

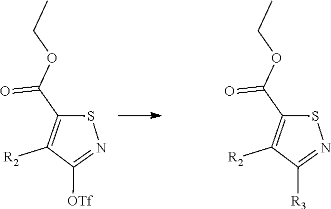

A solution of ethyl 3-(((trifluoromethyl)sulfonyl)oxy)isothiazole-5-carboxylate (1.0 eq.) and boric acid (or ester) (1.1 eq.) K₃PO₄ (3 eq.), XPhos (0.1 eq.) and XPhos Pd G3 (0.1 eq.) in dioxane is stirred at 60° C. for 4 h under nitrogen. The reaction is quenched with sat. NH₄Cl (aq.) at 0° C. and extracted with EtOAc. The combined organic layers are washed with brine, dried over Na₂SO₄, and concentrated under reduced pressure. The crude product is purified by Prep-HPLC to afford the desired compound.

### General Procedure H: For Further Carbon Bond Couplings

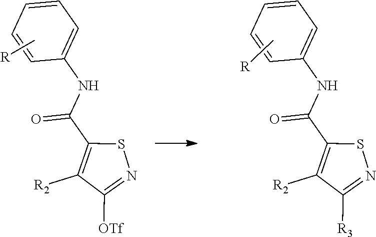

A solution of isothiazol-3-yl trifluoromethanesulfonate (1.0 eq.) and a boronic acid/ester (1.1 eq.) K₃PO₄ (3 eq.), XPhos (0.1 eq.) and XPhos Pd G3 (0.1 eq.) in dioxane is stirred at 60° C. for 4 h under nitrogen. The reaction is quenched with sat. NH₄Cl (aq.) at 0° C., and extracted with EtOAc. The combined organic layers are washed with brine, dried over Na₂SO₄, and concentrated under reduced pressure. The crude product is purified by Prep-HPLC to afford the desired compound.

Certain amine intermediates are described in the literature or are commercially available. 5-Chloro-6-(2H-1,2,3-triazol-2-yl)pyridin-3-amine.

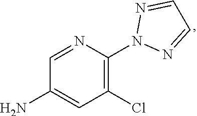 [SMILES: Nc1cnc(-n2nccn2)c(Cl)c1]

is described by Jean Quancard el al. in J. Med. Chem. 2020, 63, No. 23, 14594-14608. 5-Cyano-6-(2H-1,2,3-triazol-2-yl)pyridin-3-amine,

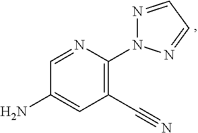 [SMILES: N#Cc1cc(N)cnc1-n1nccn1]

is described by Karen Kammertoens et al. in WO 2017/081641. 5-Methyl-6-(2H-1,2,3-triazol-2-yl)pyridin-3-amine,

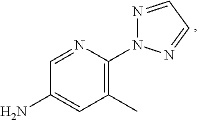

is described by Shulu Feng et al. in WO 2021/134004. 5-(Difluoromethyl)-6-(2H-1,2,3-triazol-2-yl)pyridin-3-amine,

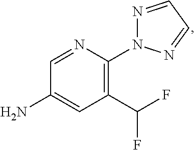 [SMILES: Nc1cnc(-n2nccn2)c(C(F)F)c1]

is described by Shulu Feng et al. in WO 2021/134004. 5-(trifluoromethyl)-6-(2H-1,2,3-triazol-2-yl)pyridin-3-amine, H2

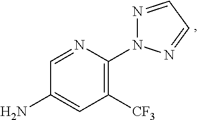 [SMILES: Nc1cnc(-n2nccn2)c(C(F)(F)F)c1]

is described by Shulu Feng et al. in WO 2021/134004. 2-(trifluoromethyl)pyridin-4-amine,

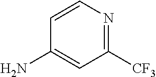 [SMILES: Nc1ccnc(C(F)(F)F)c1]

is commercially available.

Certain thiazole intermediates are described in the literature Methyl 4-iodo-3-phenylisothiazole-5-carboxylate,

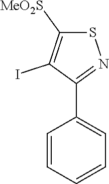 [SMILES: CS(=O)(=O)c1snc(-c2ccccc2)c1I]

is described by T. G. Murali Dhar et al. in WO 2011/059784. Methyl 3-phenyl-4-(trifluoromethyl)isothiazole-5-carboxylate,

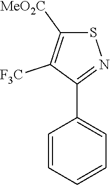 [SMILES: COC(=O)c1snc(-c2ccccc2)c1C(F)(F)F]

is described by T. G. Murali Dhar et al. in WO 2011/059784. Methyl 4-cyano-3-phenylisothiazole-5-carboxylate,

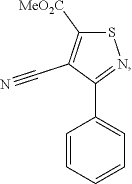 [SMILES: COC(=O)c1snc(-c2ccccc2)c1C#N]

is described by T. G. Murali Dhar et al. in WO 2011/059784.

### EXAMPLE 1—SYNTHESIS OF (4-AMINO-2-CHLOROPHENYL)(3-((TERT-BUTYLDIMETHYLSILYL)OXY)AZETIDIN-1-YL)METHANONE

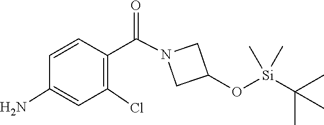 [SMILES: CC(C)(C)[Si](C)(C)OC1CN(C(=O)c2ccc(N)cc2Cl)C1]

The resulting mixture of 4-amino-2-chlorobenzoic acid (1.0 g, 5.8 mmol, 1.0 eq.), 3-((tert-butyldimethylsilyl)oxy)azetidine (1.1 g, 5.8 mmol, 1.0 eq.), HATU (3.3 g, 8.7 mmol, 1.5 eq.), DIEA (1.5 g, 11.7 mmol. 2.0 eq.) in THF (10 mL) was stirred for 16 h at room temperature under nitrogen. The mixture was then diluted with water (50 mL), and extracted with EtOAc (3×30 mL). The combined organic layers were washed with brine (1×20 mL), dried over Na₂SO₄, and concentrated under reduced pressure. The residue was purified by silica gel column chromatography, eluted with PE/EtOAc (1:1) to afford the title compound (1.5 g, 75%) as a yellow solid. (ES, m/z): [M+H]⁺ 341.

### EXAMPLE 2—SYNTHESIS OF 4-IODO-3-PHENYLISOTHIAZOLE-5-CARBOXYLIC ACID

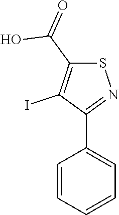 [SMILES: O=C(O)c1snc(-c2ccccc2)c1I]

General Procedure B (using methyl 4-iodo-3-phenyl-1,2-thiazole-5-carboxylate) yielded the title compound (300 mg, 63%) as a light yellow oil. (ES, m/z): [M+H]⁺ 332.

### EXAMPLE 3—SYNTHESIS OF 3-PHENYL-4-(TRIFLUOROMETHYL) ISOTHIAZOLE-5-CARBOXYLIC ACID

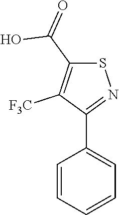 [SMILES: O=C(O)c1snc(-c2ccccc2)c1C(F)(F)F]

General Procedure B (using methyl 3-phenyl-4-(trifluoromethyl)-1,2-thiazole-5-carboxylate) yielded the title compound (480 mg, 84%) as a yellow oil. (ES, m/z): [M+H]⁺ 474.

### EXAMPLE 4—SYNTHESIS METHYL 4-METHYL-3-PHENYLISOTHIAZOLE-5-CARBOXYLATE

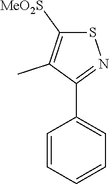 [SMILES: Cc1c(-c2ccccc2)nsc1S(C)(=O)=O]

To a stirred solution of methyl 4-iodo-3-phenyl-1,2-thiazole-5-carboxylate (700 mg, 2.0 mmol, 1.0 eq.) and trimethyl-1,3,5,2,4,6-trioxatriborinane (280 mg, 2.2 mmol, 1.1 eq.) in 1,4-dioxane (10 mL) and H₂O (1 mL) were added K₂CO₃ (700 mg, 5.1 mmol. 2.5 eq.) and Pd(dppf)Cl₂·CH₂Cl₂ (165 mg, 0.2 mmol, 0.1 eq.). The resulting mixture was stirred for 3 h at 90° C. under nitrogen. The resulting mixture was poured into water and extracted with DCM (3×20 mL). The combined organic layers were washed with H₂O (2×20 mL), dried over Na₂SO₄. After filtration, the filtrate was concentrated under reduced pressure. The residue was purified by reverse flash chromatography with the following conditions: column, C18 silica gel; mobile phase, MeCN in Water (0.1% TFA), 10% to 50% gradient in 10 min; detector, UV 254 nm, to afford the title compound (300 mg, 63%) as a brown solid. (ES, m/z): [M+H]⁺ 234.

### EXAMPLE 5—SYNTHESIS OF 4-METHYL-3-PHENYLISOTHIAZOLE-5-CARBOXYLIC ACID

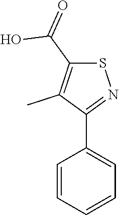

General Procedure B (using methyl 4-methyl-3-phenyl-1,2-thiazole-5-carboxylate) yielded the title compound (220 mg, 78%) as a brown solid. (ES, m/z): [M+H]⁺ 220.

### EXAMPLE 6—SYNTHESIS OF METHYL 3-PHENYL-4-(PROP-1-EN-2-YL)-1,2-THIAZOLE-5-CARBOXYLATE

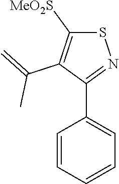 [SMILES: C=C(C)c1c(-c2ccccc2)nsc1S(C)(=O)=O]

A solution of methyl 4-iodo-3-phenyl-1,2-thiazole-5-carboxylate (700 mg, 2.0 mmol, 1.0 eq.), 4,4,5,5-tetramethyl-2-(prop-1-en-2-yl)-1,3,2-dioxaborolane (358 mg, 2.1 mmol, 1.05 eq.), Pd(dppf)Cl₂ (74.2 mg, 0.10 mmol, 0.05 eq.) and K₂CO₃ (841 mg, 6.1 mmol, 3.0 eq.) in dioxane (10 mL) and H₂O (1 mL) was stirred for 2 h at 90° C. under nitrogen. The resulting mixture was diluted with water (20 mL), and extracted with EtOAc (3×20 mL). The combined organic layers were washed with brine (10 mL), dried over anhydrous Na₂SO₄, and concentrated under reduced pressure. The residue was purified by reverse flash chromatography with the following conditions: column, C18 silica gel; mobile phase, MeCN in Water, 60% to 65% gradient in 10 min; detector, UV 254 nm, to afford the title compound (200 mg, 38%) as a pink solid. (ES, m/z): [M+H]⁺ 260.

### EXAMPLE 7—SYNTHESIS OF METHYL 4-ISOPROPYL-3-PHENYL-ISOTHIAZOLE-5-CARBOXYLATE

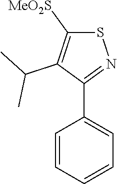 [SMILES: CC(C)c1c(-c2ccccc2)nsc1S(C)(=O)=O]

A solution of methyl 3-phenyl-4-(prop-1-en-2-yl)-1,2-thiazole-5-carboxylate (200 mg, 0.8 mmol, 1 eq.) and Pd/C (8.2 mg, 0.08 mmol, 0.1 eq.) in EtOAc (2 mL) was stirred overnight at room temperature under hydrogen. The resulting mixture was filtered, the solid was washed with EtOAc (3×10 mL), and the filtrate was concentrated under reduced pressure. This afforded the title compound (160 mg, 79%) as a light yellow oil. (ES, m/z): [M+H]⁺ 262.

### EXAMPLE 8—SYNTHESIS OF 4-ISOPROPYL-3-PHENYLISOTHIAZOLE-5-CARBOXYLIC ACID

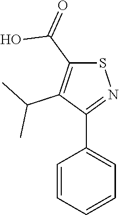 [SMILES: CC(C)c1c(-c2ccccc2)nsc1C(=O)O]

General Procedure B (using methyl 4-isopropyl-3-phenyl-1,2-thiazole-5-carboxylate) yielded the title compound (150 mg, 99%) as a light yellow oil. (ES, m/z): [M+H]⁺ 248.

### EXAMPLE 9—SYNTHESIS OF METHYL 4-CYCLOPROPYL-3-PHENYL-ISOTHIAZOLE-5-CARBOXYLATE

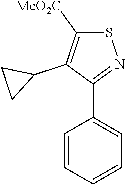 [SMILES: COC(=O)c1snc(-c2ccccc2)c1C1CC1]

A mixture of methyl 4-iodo-3-phenyl-1,2-thiazole-5-carboxylate (500 mg, 1.5 mmol, 1.0 eq.), cyclopropylboronic acid (137 mg, 1.6 mmol, 1.1 eq.), Pd(dppf)Cl₂ (106 mg, 0.15 mmol, 0.1 eq.), K₂CO₃ (600 mg, 4.3 mmol, 3.0 eq.) was suspended in dioxane (5 mL), H₂O (1 mL) at 25° C., and then stirred for 2 h at 90° C. under nitrogen. The mixture was allowed to cool down to 25° C., diluted with water (20 mL), and then extracted with EtOAc (2×40 mL). The combined organic layers were washed with brine (1×20 mL), dried over Na₂SO₄, and concentrated under reduced pressure. The residue was purified by reverse flash chromatography with the following conditions: column, C18 silica gel; mobile phase, MeCN in Water, 50% to 70% gradient in 10 min; detector, UV 254 nm to afford the title compound (100 mg, 27%). (ES, m/z): [M+H]⁺ 260.

### EXAMPLE 10—SYNTHESIS OF 4-CYCLOPROPYL-3-PHENYLISOTHIAZOLE-5-CARBOXYLIC ACID

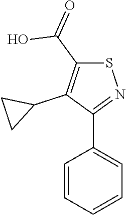 [SMILES: O=C(O)c1snc(-c2ccccc2)c1C1CC1]

General Procedure B (using methyl 4-cyclopropyl-3-phenyl-1,2-thiazole-5-carboxylate) yielded the title compound (50 mg, 59%) as a yellow oil. (ES, m/z): [M+H]⁺ 246.

### EXAMPLE 11—SYNTHESIS OF 4-CYANO-3-PHENYLISOTHIAZOLE-5-CARBOXYLIC ACID

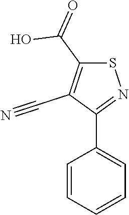 [SMILES: N#Cc1c(-c2ccccc2)nsc1C(=O)O]

General Procedure B (using methyl 4-cyano-3-phenyl-1,2-thiazole-5-carboxylate) yielded the title compound (130 mg, 69%) as a white solid. (ES, m/z): [M+H]⁺ 231.

### EXAMPLE 12—SYNTHESIS OF METHYL 4-VINYL-3-PHENYLISOTHIAZOLE-5-CARBOXYLATE

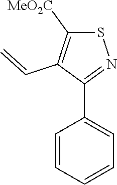 [SMILES: C=Cc1c(-c2ccccc2)nsc1C(=O)OC]

The resulting mixture of methyl 4-iodo-3-phenyl-1,2-thiazole-5-carboxylate (400 mg, 1.2 mmol, 1.0 eq.), ethenylboronic acid (90 mg, 1.3 mmol, 1.1 eq.), Pd(dppf)Cl₂CH₂Cl₂ (190 mg, 0.2 mmol, 0.2 eq.), K₂CO₃ (320 mg, 2.3 mmol, 2.0 eq.) in dioxane (10 mL) was stirred for 1 6 h at 100° C. under nitrogen. The resulting mixture was diluted with water (20 mL), and extracted with EtOAc (3×20 mL). The combined organic layers were washed with brine (10 mL), dried over anhydrous Na₂SO₄ and concentrated under reduced pressure. The residue was purified by silica gel column chromatography, eluted with PE/EtOAc (3:1) to the title compound (200 mg, 70%) as a yellow solid. (ES, m/z): [M+H]⁺ 246.

### EXAMPLE 13—SYNTHESIS OF 4-VINYL-3-PHENYLISOTHIAZOLE-5-CARBOXYLIC ACID

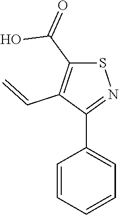 [SMILES: C=Cc1c(-c2ccccc2)nsc1C(=O)O]

General Procedure B (using methyl 3-phenyl-4-vinylisothiazole-5-carboxylate) yielded the title compound (150 mg, 80%) as a white solid. (ES, m/z): [M+H]⁺ 232.

### EXAMPLE 14—SYNTHESIS OF METHYL 4-ETHYL-3-PHENYLISOTHIAZOLE-5-CARBOXYLATE

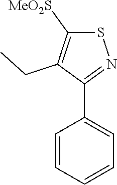 [SMILES: CCc1c(-c2ccccc2)nsc1S(C)(=O)=O]

A suspension of methyl 4-ethenyl-3-phenyl-1,2-thiazole-5-carboxylate (160 mg, 0.7 mmol, 1.0 eq.) and Pd/C (50 mg, 0.5 mmol, 0.7 eq.) in EtOH (5 mL) was stirred for 16 h at room temperature under hydrogen. The resulting mixture was filtered, the solid was washed with EtOH (5×5 mL), and the filtrate was concentrated under reduced pressure to afford the title intermediate (150 mg, 93%) as a black solid. (ES, m/z): [M+H]⁺ 248.

### EXAMPLE 15—SYNTHESIS OF 4-ETHYL-3-PHENYLISOTHIAZOLE-5-CARBOXYLIC ACID

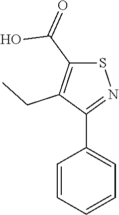 [SMILES: CCc1c(-c2ccccc2)nsc1C(=O)O]

General Procedure B (using methyl 4-ethyl-3-phenylisothiazole-5-carboxylate) yielded the title compound (130 mg, 92%) as a white solid. (ES, m/z): [M+H]⁺ 234.

### EXAMPLE 16—SYNTHESIS OF (Z)-3-CHLORO-N-HYDROXYPICOLINIMIDOYL CYANIDE

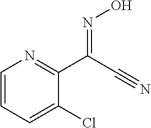 [SMILES: N#C/C(=N\O)c1ncccc1Cl]

To a stirred solution of (Z)-3-chloro-N-hydroxy-pyridine-2-carboximidoyl chloride (2.9 g, 15 mmol, 1.0 eq) in diethyl ether (40 mL) and water (15 mL) was added potassium cyanide (2.0 g, 31 mmol, 2.0 eq) and the mixture stirred at room temperature for 16 h. The solution was diluted with water (15 mL) and extracted with EtOAc (3×100 mL). The organics were combined, dried over sodium sulfate, and concentrated under reduced pressure yielding 520 mg of crude material containing little desired product by LCMS. To the aqueous layer was added NaCl which resulted in precipitation of a large amount of solid, which was filtered and triturated with MeOH. The suspension was filtered and the filtrate concentrated under reduced pressure yielding 8 g of crude material. This material was dissolved in a minimum amount of water and extracted using 20% MeOH in EtOAc (12×200 mL). The organics were combined, dried over sodium sulfate, filtered, and concentrated under reduced pressure yielding the title compound (3.4 g, 121%) as crude orange solid, which was used in the next step without further purification. (ES, m/z): [M+H]⁺ 181; ¹H NMR (400 MHz, DMSO-d₆) δ 7.06 (dd, J=4.6, 8.0 Hz, 1H), 7.80 (dd, J=1.5, 7.9 Hz, 1H), 8.41 (dd, J=1.5, 4.6 Hz, 1H), OH not visible.

### EXAMPLE 17—SYNTHESIS OF (Z)-3-CHLORO-N-(TOSYLOXY)PICOLINIMIDOYL CYANIDE

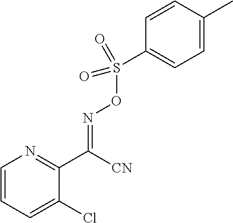 [SMILES: Cc1ccc(S(=O)(=O)O/N=C(\C#N)c2ncccc2Cl)cc1]

To a solution of (Z)-3-chloro-N-hydroxypicolinimidoyl cyanide (2.8 g, 15 mmol, 1.0 eq) and triethylamine (5.4 mL, 39 mmol, 2.5 eq) in dichloromethane (90 mL) stirring under nitrogen at −10° C. was added p-toluenesulfonyl chloride (4.4 g, 23 mmol, 1.5 eq). The resulting mixture was slowly allowed to reach room temperature and stirred for 16 h. The mixture was filtered before concentrating the filtrate under reduced pressure. The crude residue was purified using silica column chromatography (isocratic 20% EtOAc in cyclohexane). Fractions containing product were concentrated under reduced pressure yielding the title compound (1.3 g, 26%) as an off-white solid. (ES, m/z): [M+H]⁺ 335; ¹H NMR (400 MHz, DMSO-d₆) δ 2.44 (s, 3H), 7.56 (d, J=8.2 Hz, 2H), 7.67 (dd, J=4.6, 8.2 Hz, 1H), 7.98 (d, J=8.5 Hz, 2H), 8.16 (dd, J=1.3, 8.2 Hz, 1H), 8.73 (dd, J=1.4, 4.5 Hz, 1H).

### EXAMPLE 18—SYNTHESIS OF METHYL 4-AMINO-3-(3-CHLOROPYRIDIN-2-YL)ISOTHIAZOLE-5-CARBOXYLATE

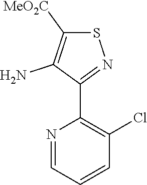 [SMILES: COC(=O)c1snc(-c2ncccc2Cl)c1N]

To a solution of (Z)-3-chloro-N-(tosyloxy)picolinimidoyl cyanide (1.3 g, 4.0 mmol, 1.0 eq) in anhydrous methanol (40 mL) was added methyl thioglycolate (0.39 mL, 4.4 mmol, 1.1 eq) and then triethylamine (1.2 mL, 8.8 mmol, 2.2 eq). The reaction mixture was stirred at room temperature for 16 h. The solvent was removed under reduced pressure and the crude residue was purified by silica column chromatography (0 to 50% EtOAc in cyclohexane). Fractions containing product were combined and concentrated under reduced pressure to yield the title compound (693 mg, 64%) as a yellow solid. (ES, m/z): [M+H]⁺ 269; ¹H NMR (400 MHz, DMSO-d₆) δ 3.87 (s, 3H), 6.66 (s, 2H), 7.59 (dd, J=4.6, 8.3 Hz, 1H), 8.17 (dd, J=1.4, 8.3 Hz, 1H), 8.69 (dd, J=1.4, 4.6 Hz, 1H).

### EXAMPLE 19—SYNTHESIS OF METHYL 3-(3-CHLOROPYRIDIN-2-YL)-4-IODOISOTHIAZOLE-5-CARBOXYLATE

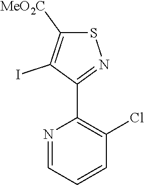 [SMILES: COC(=O)c1snc(-c2ncccc2Cl)c1I]

To a solution of methyl 4-amino-3-(3-chloropyridin-2-yl)isothiazole-5-carboxylate (693 mg, 2.6 mmol, 1.0 eq) in chloroform (20 mL) was added iodine (3.5 g, 13.6 mmol, 5.3 eq) and isopentyl nitrite (0.52 mL, 3.9 mmol, 1.5 eq) the reaction was then refluxed for 30 minutes. LCMS showed complete conversion to product. The reaction was allowed to cool to room temperature and was diluted with DCM and washed with 1 M Na₂S₂O₃ and brine. The organics were then dried and concentrated. The crude residue was purified using flash column chromatography (0 to 100% EtOAc in cyclohexane). Fractions containing product were combined and concentrated to yield the title compound (290 mg, 0.76 mmol, 30%) as a red oil. (ES, m/z): [M+H]⁺ 380; ¹H NMR (400 MHz, DMSO-d₆) δ 3.95 (s, 3H), 7.66 (dd, J=4.7, 8.2 Hz, 1H), 8.20 (dd, J=1.4, 8.2 Hz, 1H), 8.72 (dd, J=1.4, 4.7 Hz, 1H).

### EXAMPLE 20—SYNTHESIS OF METHYL 3-(3-CHLOROPYRIDIN-2-YL)-4-(TRIFLUOROMETHYL)ISOTHIAZOLE-5-CARBOXYLATE

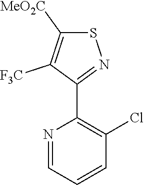 [SMILES: COC(=O)c1snc(-c2ncccc2Cl)c1C(F)(F)F]

To a solution of methyl 3-(3-chloropyridin-2-yl)-4-iodoisothiazole-5-carboxylate (290 mg, 0.76 mmol, 1.0 eq) in NN-dimethylformamide (4 mL) was added copper(I) iodide (290 mg, 1.5 mmol, 2.0 eq) and methyl 2,2-difluoro-2-(fluorosulfonyl)acetate (0.19 mL, 1.5 mmol, 2.0 eq) and the reaction stirred at 90° C. for 3 h. The reaction mixture was allowed to cool to room temperature and the solvent removed under reduced pressure. The crude product was purified using flash column chromatography, eluting with 0 to 80% EtOAc in cyclohexane. Fractions containing product were combined and concentrated under reduced pressure to yield the title compound (150 mg, 610%) as a yellow oil. (ES, m/z): [M+H]⁺ 322; ¹H NMR (400 MHz, DMSO-d₆) δ 3.99 (s, 3H), 7.67 (dd, J=4.8, 8.3 Hz, 1H), 8.20 (dd, J=1.4, 8.3 Hz, 1H), 8.68 (dd, J=1.4, 4.6 Hz, 1H).

### EXAMPLE 21—SYNTHESIS OF 3-(3-CHLOROPYRIDIN-2-YL)-4-(TRIFLUORO-METHYL)ISOTHIAZOLE-5-CARBOXYLIC ACID

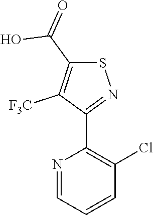 [SMILES: O=C(O)c1snc(-c2ncccc2Cl)c1C(F)(F)F]

To a solution of methyl 3-(3-chloropyridin-2-yl)-4-(trifluoromethyl)isothiazole-5-carboxylate (100 mg, 0.31 mmol, 1.0 eq) in MeOH (2.4 mL) was added lithium hydroxide monohydrate (14 mg, 0.33 mmol, 1.05 eq) in water (0.6 mL), and the reaction mixture was stirred for 4 h. The solvent was removed under reduced pressure to afford the title compound (Li salt, 103 mg, 106%) as an off-white solid. (ES, m/z): [M+H]⁺ 308; ¹H NMR (400 MHz, DMSO-d₆) δ 7.57 (1H, dd, J=4.6, 8.3 Hz), 8.11 (1H, dd, J=1.4, 8.3 Hz), 8.62 (1H, dd, J=1.4, 4.7 Hz).

### EXAMPLE 22—SYNTHESIS OF METHYL 3-(TETRAHYDRO-2H-PYRAN-4-YL)-4-(TRIFLUOROMETHYL) ISOTHIAZOLE-5-CARBOXYLATE

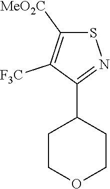 [SMILES: COC(=O)c1snc(C2CCOCC2)c1C(F)(F)F]

A mixture of methyl 4-iodo-3-(oxan-4-yl)-1,2-thiazole-5-carboxylate (140 mg, 0.4 mmol, 1.0 eq.), methyl 2,2-difluoro-2-sulfoacetate (152 mg, 0.8 mmol, 2.0 eq.) and copper(I) iodide (150 mg, 0.8 mmol, 2.0 eq.) in DMF (2 mL) was stirred for 3 h at 90° C. under nitrogen. The precipitate was collected by filtration and washed with EtOAc (3×3 mL). The filtrate was partitioned with water, and the aqueous layer was extracted with EtOAc (3×5 mL). The combined organic layers were washed with brine (10 mL), dried over anhydrous Na₂SO₄, and concentrated under reduced pressure. The residue was purified by reversed-phase flash chromatography with the following conditions: column, C18 silica gel; mobile phase, MeCN in Water (0.1% NH₃·H₂O), 10% to 50% gradient in 10 min; detector, UV 254 nm to afford the title ester (110 mg, 93%) as a yellow oil. (ES, n/z): [M+H]⁺ 296.0; ¹H NMR (400 MHz, CDCl₃): δ 1.22-1.30 (m, 2H), 1.80-1.90 (m, 1H), 1.90-2.05 (m, 1H), 3.25-3.40 (m, 1H), 3.50-3.65 (m, 2H), 3.96 (s, 3H), 4.05-4.15 (m, 2H).

### EXAMPLE 23—SYNTHESIS OF 3-(TETRAHYDRO-2H-PYRAN-4-YL)-4-(TRIFLUOROMETHYL)ISOTHIAZOLE-5-CARBOXYLIC ACID

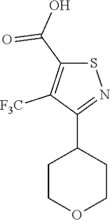 [SMILES: O=C(O)c1snc(C2CCOCC2)c1C(F)(F)F]

General Procedure B (using methyl 3-(tetrahydro-2H-pyran-4-yl)-4-(trifluoromethyl)isothiazole-5-carboxylate) yielded the title compound (100 mg, quant.) as a yellow solid. (ES, m/z): [M+H]⁺ 282.0; ¹H NMR (400 MHz, CDCl₃): δ 1.21-1.30 (m, 2H), 1.70-1.80 (m, 1H), 1.80-1.95 (m, 1H), 3.20-3.35 (m, 1H), 3.50-3.60 (m, 2H), 4.10-4.25 (m, 2H).

### EXAMPLE 24—SYNTHESIS OF METHYL 3-ISOPROPYL-4-(TRIFLUORO-METHYL)ISOTHIAZOLE-5-CARBOXYLATE

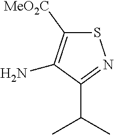

A mixture of methyl 4-iodo-3-isopropyl-1,2-thiazole-5-carboxylate (1.00 g, 3.2 mmol, 1.0 eq.), methyl 2,2-difluoro-2-sulfoacetate (1.2 g, 6.4 mmol, 2.0 eq.) and copper(I) iodide (1.2 g, 6.4 mmol, 2.0 eq.) in DMF (10 mL) was stirred for 3 h at 90° C. under nitrogen. The resulting mixture was filtered, and the precipitate was washed with EtOAc (2×5 mL). The filtrate was concentrated under reduced pressure. The residue was purified by reverse phase flash with the following conditions (90% of MeCN) to afford the title ester (500 mg, 61%) as a yellow oil. (ES, m/z): [M+H]⁺ 254.0; ¹H NMR (400 MHz, CDCl₃): δ 1.59 (d, J=6.8 Hz, 6H), 1.70-1.90 (m, 1H), 3.97 (s, 3H).

### EXAMPLE 25—SYNTHESIS OF 3-ISOPROPYL-4-(TRIFLUOROMETHYL) ISOTHIAZOLE-5-CARBOXYLIC ACID

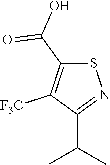

General Procedure B (using methyl 3-isopropyl-4-(trifluoromethyl)isothiazole-5-carboxylate) yielded the title compound (230 mg, crude) as a yellow solid. (ES, m/z): [M+H]⁺ 240.0; ¹H NMR (400 MHz, CDCl₃): δ 1.53 (d, J=6.8 Hz, 6H), 1.60-1.80 (m, 1H), 13.58 (s, 1H).

### EXAMPLE 26—SYNTHESIS OF (5-(METHOXYCARBONYL)-3-PHENYL-ISOTHIAZOL-4-YL)BORONIC ACID

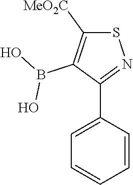 [SMILES: COC(=O)c1snc(-c2ccccc2)c1B(O)O]

A mixture of methyl 4-iodo-3-phenyl-1,2-thiazole-5-carboxylate (500 mg, 1.5 mmol, 1 eq.), trimethyl borate (151 mg, 1.5 mmol, 1.0 eq.), i-PrMgCl—LiCl (1.3 M in THF) (324 mg, 2.2 mmol, 1.5 eq.) in THF (10 mL) was stirred for 4 hat −40° C. under nitrogen. The reaction was quenched with water (20 mL) at room temperature, and the resulting mixture was extracted with DCM (3×30 mL). The combined organic layers were washed with brine (10 mL), dried over anhydrous Na₂SO₄, and concentrated under reduced pressure. The residue was purified by Prep-TLC (PE/EtOAc 1:1) to afford the title compound (230 mg, 60%) as an off-white solid. (ES, m/z): [M+H]⁺ 264.0.

### EXAMPLE 27—SYNTHESIS OF METHYL 4-HYDROXY-3-PHENYLISOTHIAZOLE-5-CARBOXYLATE

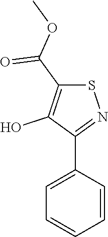 [SMILES: COC(=O)c1snc(-c2ccccc2)c1O]

A mixture of 5-(methoxycarbonyl)-3-phenyl-1,2-thiazol-4-ylboronic acid (210 mg, 0.8 mmol, 1 eq.), NaBO₃ (196 mg, 2.4 mmol, 3.0 eq.) in MeOH (5 mL)/H₂O (1 mL) was stirred overnight at room temperature under nitrogen. The resulting mixture was concentrated under reduced pressure, and then was extracted with EtOAc (3×10 mL). The combined organic layers were washed with brine (7 mL), dried over anhydrous Na₂SO₄, and concentrated under reduced pressure. The residue was purified by Prep-TLC (DCM/MeOH 10:1) to afford the title compound (180 mg, 96%) as an off-white solid. (ES, m/z): [M+H]⁺ 236.

### EXAMPLE 28—SYNTHESIS OF METHYL 4-METHOXY-3-PHENYLISOTHIAZOLE-5-CARBOXYLATE

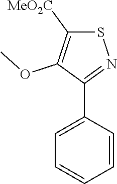 [SMILES: COC(=O)c1snc(-c2ccccc2)c1OC]

A mixture of 4-hydroxy-3-phenylisothiazole-5-carboxylic acid (160 mg, 0.7 mmol, 1 eq.) and methyl iodide (310 mg, 2.2 mmol, 3 eq.), K₂CO₃ (300 mg, 2.2 mmol, 3 eq.) in DMF (3 mL) was stirred overnight at 80° C. under nitrogen. The resulting mixture was diluted with water (5 mL), and extracted with EtOAc (3×10 mL). The combined organic layers were washed with brine (5 mL), dried over anhydrous Na₂SO₄, and concentrated under reduced pressure to afforded the title compound (160 mg, 89%) as an off-white solid. (ES, m/z): [M+H]⁺ 250.0.

### EXAMPLE 29—SYNTHESIS OF 4-METHOXY-3-PHENYLISOTHIAZOLE-5-CARBOXYLIC ACID

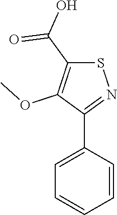 [SMILES: COc1c(-c2ccccc2)nsc1C(=O)O]

General Procedure B (using methyl 4-methoxy-3-phenylisothiazole-5-carboxylate) yielded the title compound (130 mg, 86%) as an off-white solid. (ES, m/z): [M+H]⁺ 236.0.

### EXAMPLE 30—SYNTHESIS OF METHYL 3-(1-METHYL-6-OXO-1,6-DIHYDRO-PYRIDIN-3-YL)-4-(TRIFLUOROMETHYL)ISOTHIAZOLE-5-CARBOXYLATE

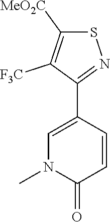 [SMILES: COC(=O)c1snc(-c2ccc(=O)n(C)c2)c1C(F)(F)F]

A mixture of methyl 4-iodo-3-(1-methyl-6-oxo-1,6-dihydropyridin-3-yl)isothiazole-5-carboxylate (290 mg, 0.8 mmol, 1 eq.), methyl 2,2-difluoro-2-sulfoacetate (296 mg, 1.5 mmol, 2 eq.) and copper(I) iodide (294 mg, 1.5 mmol, 2 eq.) in DMF (5 mL) was stirred for 1 h at 90° C. under nitrogen. The resulting mixture was extracted with EtOAc (3×10 mL), the combined organic layers were washed with dioxane (10 mL), dried over anhydrous Na₂SO₄, and concentrated under reduced pressure. This resulted in the title compound (150 mg, 61%) as a off-white solid. (ES, m/z): [M+H]⁺ 319.0.

### EXAMPLE 31—SYNTHESIS OF 3-(1-METHYL-6-OXO-1,6-DIHYDROPYRIDIN-3-YL)-4-(TRIFLUOROMETHYL)ISOTHIAZOLE-5-CARBOXYLIC ACID

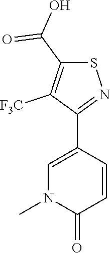 [SMILES: Cn1cc(-c2nsc(C(=O)O)c2C(F)(F)F)ccc1=O]

General Procedure B (using methyl 3-(1-methyl-6-oxo-1,6-dihydropyridin-3-yl)-4-(trifluoromethyl)isothiazole-5-carboxylate) yielded the title compound (69 mg, 84%) as an off-white solid. (ES, m/z): [M+H]⁺ 305.0.

### EXAMPLE 32—SYNTHESIS OF METHYL 3-(1-METHYL-1H-PYRAZOL-4-YL)-4-(TRIFLUOROMETHYL) ISOTHIAZOLE-5-CARBOXYLATE

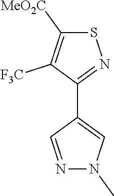 [SMILES: COC(=O)c1snc(-c2cnn(C)c2)c1C(F)(F)F]

A solution of methyl 3-(1-methyl-1H-pyrazol-4-yl)-4-(trifluoromethyl)isothiazole-5-carboxylate (220 mg, 0.6 mmol, 1 eq.), CuI (240 mg, 1.3 mmol, 2 eq.) and methyl 2,2-difluoro-2-sulfoacetate (242 mg, 1.3 mmol, 2 eq.) in DMF (2 mL) was stirred for 2 h at 90° C. under nitrogen. The resulting mixture was filtered, and the solids were washed with MeOH (3×10 mL). The filtrate was concentrated under reduced pressure, and the residue was purified by reversed-phase flash chromatography with the following conditions: column, C18 silica gel; mobile phase, MeCN in Water, 50% to 60% gradient in 10 mm: detector, UV 254 nm. This resulted in the title ester (130 mg, 710%) as a light yellow oil. (ES, m/z): [M+H]⁺ 292.1.

### EXAMPLE 33—SYNTHESIS OF 3-(1-METHYL-1H-PYRAZOL-4-YL)-4-(TRIFLUOROMETHYL)ISOTHIAZOLE-5-CARBOXYLIC ACID

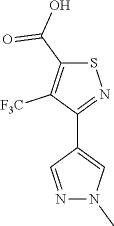 [SMILES: Cn1cc(-c2nsc(C(=O)O)c2C(F)(F)F)cn1]

General Procedure B (using methyl 3-(1-methyl-1H-pyrazol-4-yl)-4-(trifluoromethyl)isothiazole-5-carboxylate) yielded the title compound (100 mg, 81%) as a light yellow solid. (ES, m/z): [M+H]⁺ 278.0.

### EXAMPLE 34—SYNTHESIS OF (E)-N-(TOSYLOXY)IMIDAZO[1,2-A]PYRIDINE-3-CARBIMIDOYL CYANIDE

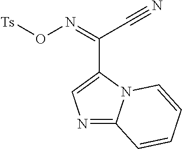 [SMILES: Cc1ccc(S(=O)(=O)O/N=C(/C#N)c2cnc3ccccn23)cc1]

A solution of (E)-imidazo[1,2-a]pyridine-3-carbonimidoyl cyanide (630 mg, 3.4 mmol, 1 eq.) and TsCl (645 mg, 3.4 mmol, 1 eq.) in toluene (7 mL) was stirred for 2 h at 110° C. under nitrogen. The mixture was allowed to cool down to room temperature, and then was diluted with H₂O and extracted with EtOAc (3×20 mL). The combined organic layers were washed with brine (50 mL), dried over anhydrous Na₂SO₄, and concentrated under reduced pressure. This resulted in the title compound (710 mg, quant.) as a light yellow solid. (ES, m/z): [M+H]⁺ 341.

### EXAMPLE 35—SYNTHESIS OF METHYL 4-AMINO-3-(IMIDAZO[1,2-A]PYRIDIN-3-YL)ISOTHIAZOLE-5-CARBOXYLATE

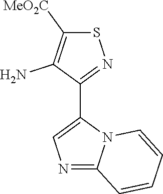 [SMILES: COC(=O)c1snc(-c2cnc3ccccn23)c1N]

A solution of (E)-N-(tosyloxy)imidazo[1,2-a]pyridine-3-carbimidoyl cyanide (700 mg, 2.1 mmol, 1 eq.), methyl thioglycolate (262 mg, 2.5 mmol, 1.2 eq.) and TEA (416 mg, 4.1 mmol, 2 eq.) in MeOH (8 mL) was stirred for 3 h at room temperature under nitrogen. The resulting mixture was diluted with H₂O (30 mL) and extracted with EtOAc (3×20 mL). The combined organic layers were washed with brine (50 mL), dried over anhydrous Na₂SO₄, and concentrated under reduced pressure. The residue was purified by reversed-phase flash chromatography. This resulted in the title compound (140 mg, 25%) as a brown solid. (ES, m/z): [M+H]⁺ 275.

### EXAMPLE 36—SYNTHESIS OF METHYL 3-(IMIDAZO[1,2-A]PYRIDIN-3-YL)-4-IODOISOTHIAZOLE-5-CARBOXYLATE

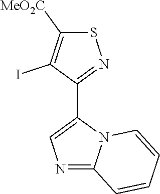 [SMILES: COC(=O)c1snc(-c2cnc3ccccn23)c1I]

A solution of methyl 4-amino-3-(imidazo[1,2-a]pyridin-3-yl)isothiazole-5-carboxylate (130 mg, 0.5 mmol, 1 eq.), a spiral (83 mg, 0.7 mmol, 1.5 eq.), and I₂ (638 mg, 2.5 mmol, 5.3 eq.) in CHCl₃ (2 mL) was stirred for 2 h at 60° C. under nitrogen. The mixture was allowed to cool down to room temperature and then was quenched by the addition of saturated sodium hyposulfite (10 mL). The resulting mixture was extracted with EtOAc (3×10 mL). The combined organic layers were washed with brine (30 mL), dried over anhydrous Na₂SO₄, and concentrated under reduced pressure. This resulted in the title compound (140 mg, quant.) as a light yellow solid. (ES, m/z): [M+H]+ 386.

### EXAMPLE 37—SYNTHESIS OF METHYL 3-(IMIDAZO[1,2-A]PYRIDIN-3-YL)-4-(TRIFLUOROMETHYL) ISOTHIAZOLE-5-CARBOXYLATE

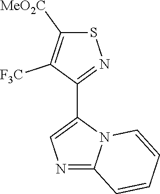 [SMILES: COC(=O)c1snc(-c2cnc3ccccn23)c1C(F)(F)F]

To a stirred solution of methyl 3-(imidazo[1,2-a]pyridin-3-yl)-4-iodoisothiazole-5-carboxylate (100 mg, 0.03, 1 eq.) and methyl 2,2-difluoro-2-sulfoacetate (299 mg, 1.6 mmol, 6 eq.) in DMF (3 mL) was added copper(I) iodide (99 mg, 0.5 mmol, 2 eq.) at room temperature. The resulting mixture was stirred for 1 h at 90° C. under nitrogen. The resulting mixture was diluted with H₂O (30 mL) and extracted with EtOAc (3×20 mL). The combined organic layers were washed with brine (50 mL), dried over anhydrous Na₂SO₄, and concentrated under reduced pressure. This resulted in the title ester (120 mg, quant.) as a light yellow solid. (ES, m/z): [M+H]⁺ 328.

### EXAMPLE 38—SYNTHESIS OF 3-(IMIDAZO[1,2-A]PYRIDIN-3-YL)-4-(TRIFLUOROMETHYL)ISOTHIAZOLE-5-CARBOXYLIC ACID

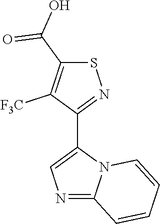 [SMILES: O=C(O)c1snc(-c2cnc3ccccn23)c1C(F)(F)F]

General Procedure B (using methyl 3-(imidazo[1,2-a]pyridin-3-yl)-4-(trifluoromethyl)isothiazole-5-carboxylate) yielded the title compound (85 mg, quant.) as a brown solid. (ES, m/z): [M+H]⁺ 314.

### EXAMPLE 39—SYNTHESIS OF ETHYL 3-(QUINOLIN-5-YL)-4-(TRIFLUORO-METHYL)ISOTHIAZOLE-5-CARBOXYLATE

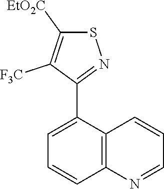 [SMILES: CCOC(=O)c1snc(-c2cccc3ncccc23)c1C(F)(F)F]

A mixture of ethyl 3-(trifluoromethanesulfonyloxy)-4-(trifluoromethyl)-1,2-thiazole-5-carboxylate (200 mg, 0.5 mmol, 1 eq.), quinolin-5-ylboronic acid (139 mg, 0.8 mmol, 1.5 eq.), K₃PO₄ (341 mg, 1.6 mmol, 3 eq.), XPhos (26 mg, 0.05 mmol, 0.1 eq.) and XPhos Pd G3 (45 mg, 0.05 mmol, 0.1 eq.) in dioxane (5 mL) was stirred for 2 h at 60° C. under nitrogen atmosphere. The reaction was quenched with water at 25° C., and the resulting mixture was extracted with EtOAc (2×50 mL). The combined organic layers were washed with brine (2×50 mL), dried over anhydrous MgSO₄, and concentrated under reduced pressure. The residue was purified by reversed-phase flash chromatography with the following conditions: column, C18 silica gel; mobile phase, MeCN in Water (10 mmol/L NH₄HCO₃), 30% to 60% gradient in 10 min; detector, UV 254 nm to afford the title ester (150 mg, 79%) as a yellow solid. (ES, m/z): [M+H]⁺ 353.0; ¹H NMR (400 MHz, DMSO-d₆): δ 1.36 (t, J=7.1 Hz, 3H), 4.46 (q, J=7.1 Hz, 2H), 7.63 (dd, J=8.6, 4.3 Hz, 1H), 7.74 (dd, J=7.2, 1.1 Hz, 1H), 7.94 (dd, J=8.6, 7.1 Hz, 1H), 8.08 (d, J=8.4 Hz, 1H), 8.24 (d, J=8.5 Hz, 1H), 9.04 (dd, J=4.3, 1.6 Hz, 1H).

### EXAMPLE 40—SYNTHESIS OF 3-(QUINOLIN-5-YL)-4-(TRIFLUOROMETHYL) ISOTHIAZOLE-5-CARBOXYLIC ACID

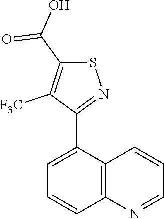 [SMILES: O=C(O)c1snc(-c2cccc3ncccc23)c1C(F)(F)F]

General Procedure B (using ethyl 3-(quinolin-5-yl)-4-(trifluoromethyl)isothiazole-5-carboxylate) yielded the title compound (115 mg, 96%) as a white solid. (ES, m/z): [M+H]⁺ 325.0; ¹H NMR (400 MHz, DMSO-d₆): δ 7.59-7.50 (m, 1H), 7.66 (d, J=7.0 Hz, 1H), 7.98-7.83 (m, 2H), 8.19 (dd, J=8.5, 4.9 Hz, 1H), 8.97 (dd, J=4.2, 1.6 Hz, 1H).

### EXAMPLE 41—SYNTHESIS OF (4-AMINO-2-CHLOROPHENYL)(3-HYDROXY-AZETIDIN-1-YL)METHANONE

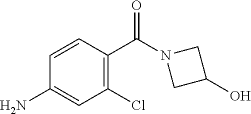 [SMILES: Nc1ccc(C(=O)N2CC(O)C2)c(Cl)c1]

A mixture of 4-amino-2-chlorobenzoic acid (400 mg, 2.2 mmol, 1.0 eq.), azetidin-3-ol (160 mg, 2.1 mmol, 0.9 eq.), HATU (700 mg, 3.7 mmol, 1.6 eq.), and DIEA (450 mg, 8.0 mmol, 3.0 eq.) in THF (12 mL) was stirred overnight at room temperature under nitrogen. The resulting mixture was diluted with water (10 mL), and extracted with EtOAc (3×10 mL). The combined organic layers were washed with brine (1×5 mL), dried over anhydrous Na₂SO₄, and concentrated under reduced pressure. The residue was purified by silica gel column chromatography, eluted with DCM/MeOH (10:1) to afford the title compound (300 mg, 57%) as a yellow solid. (ES, m/z): [M+H]⁺ 227.0.

### EXAMPLE 42—SYNTHESIS OF 2-(MORPHOLIN-4-YL)-5-NITROPYRIDINE-3-CARBONITRILE

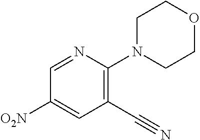 [SMILES: N#Cc1cc([N+](=O)[O-])cnc1N1CCOCC1]

A solution of 2-chloro-5-nitropyridine-3-carbonitrile (500 mg, 2.7 mmol, 1.0 eq.), morpholine (261 mg, 3.0 mmol, 1.1 eq.) and Cs₂CO₃ (2.7 g, 8.2 mmol, 3.0 eq.) in ACN (8 mL) was stirred for 2 h at 40° C. The reaction was quenched with sat. NaHCO₃ (aq.) at room temperature, and extracted with EtOAc (3×100 mL). The combined organic layers were washed with brine (3×100 mL), dried over Na₂SO₄, and concentrated under reduced pressure. This resulted in the title compound (600 mg, 94%) as a yellow solid. (ES, m/z): [M+H]⁺ 235.1.

### EXAMPLE 43—SYNTHESIS OF 5-AMINO-2-(MORPHOLIN-4-YL)PYRIDINE-3-CARBONITRILE

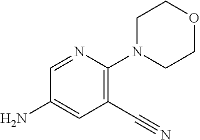 [SMILES: N#Cc1cc(N)cnc1N1CCOCC1]

To a stirred solution of 2-(morpholin-4-yl)-5-nitropyridine-3-carbonitrile (300 mg, 1.3 mmol, 1.0 eq.), Fe (358 mg, 6.4 mmol, 5.0 eq.) and NH₄Cl (343 mg, 6.4 mmol, 5.0 eq.) in EtOH (10 mL) was added AcOH (77 mg, 1.3 mmol, 1.0 eq.) dropwise. The resulting mixture was stirred for 2 h at 45° C., and then neutralized to pH 7 with NH₃·H₂O. The resulting mixture was filtered, the solids were washed with EtOAc (5×100 mL), and the filtrate was concentrated under reduced pressure. This resulted in the title compound (250 mg, 96%) as an orange solid. (ES, m/z): [M+H]⁺ 205.1.

### EXAMPLE 44—SYNTHESIS OF 2-(4-METHYLPIPERAZIN-1-YL)-5-NITRO-NICOTINONITRILE

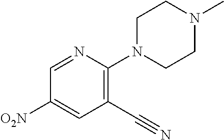 [SMILES: CN1CCN(c2ncc([N+](=O)[O-])cc2C#N)CC1]

A solution of 2-chloro-5-nitropyridine-3-carbonitrile (500 mg, 2.7 mmol, 1.0 eq.), 1-methylpiperazine (300 mg, 3.0 mmol, 1.1 eq.) and Cs₂CO₃ (2.7 g, 8.2 mmol, 3.0 eq.) in ACN (5 mL) was stirred for 2 h at 45° C. The resulting mixture was poured into water (50 mL) and extracted with EtOAc (2×50 mL). The combined organic layers were washed with brine (4×100 mL), dried over Na₂SO₄, and concentrated under reduced pressure. This resulted in the title compound (600 mg, 89%) as a yellow solid. (ES, m/z): [M+H]⁺ 248.0; ¹H NMR (400 MHz, DMSO-d₆): δ 2.22 (s, 3H), 2.44-2.48 (m, 4H), 3.90-4.01 (m, 4H), 8.83 (d, J=2.8 Hz, 1H), 9.10 (d, J=2.8 Hz, 1H).

### EXAMPLE 45—SYNTHESIS OF 5-AMINO-2-(4-METHYLPIPERAZIN-1-YL) NICOTINONITRILE

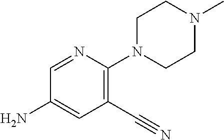 [SMILES: CN1CCN(c2ncc(N)cc2C#N)CC1]

To a stirred solution of 2-(4-methylpiperazin-1-yl)-5-nitropyridine-3-carbonitrile (300 mg, 1.2 mmol, 1.0 eq.) and Fe (23 mg, 0.4 mmol, 5.0 eq.) in EtOH (3 mL) was added NH₄Cl (325 mg, 6.1 mmol, 5.0 eq.). The resulting mixture was stirred for 1 h at 80° C. The resulting mixture was concentrated under reduced pressure. The resulting mixture was filtered, the filter cake was washed with DCM (3×10 mL). The filtrate was concentrated and poured into water (20 mL) and neutralized to pH 7 with NH₃·H₂O and extracted with EtOAc (2×50 mL). The combined organic layers were washed with brine (4×100 mL), dried over Na₂SO₄, and concentrated under reduced pressure. This resulted in the title compound (250 mg, 95%) as a yellow oil. (ES, m/z): [M+H]⁺ 217.9.

### EXAMPLE 46—SYNTHESIS OF N-(3-CYANO-5-NITROPYRIDIN-2-YL)-N-METHYLACETAMIDE

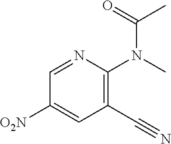 [SMILES: CC(=O)N(C)c1ncc([N+](=O)[O-])cc1C#N]

To a stirred solution of 2-(methylamino)-5-nitropyridine-3-carbonitrile (1.4 g, 7.9 mmol, 1.0 eq.) and DMAP (192 mg, 1.6 mmol, 0.2 eq.) in pyridine (15 mL) was added acetic anhydride (1.6 g, 16 mmol, 2.0 eq.) dropwise at room temperature, and the resulting mixture was stirred for 3 h at 80° C. The reaction mixture was then poured into water (200 mL) and extracted with EtOAc (2×200 mL). The combined organic layers were washed with brine (400 mL), dried over anhydrous Na₂SO₄, and concentrated under reduced pressure. The residue was purified by reversed-phase flash chromatography with the following conditions: column, C18 silica gel; mobile phase, MeCN in Water (0.1% TFA), 10% to 50% gradient in 10 min; detector, UV 254 nm to afford the title compound (680 mg, 39%) as a yellow solid. (ES, m/z): [M−H]⁻ 221.0; ¹H NMR (400 MHz, DMSO-d₆): δ 2.27 (s, 3H), 3.45 (s, 3H), 9.26 (d, J=2.7 Hz, 1H), 9.51 (d, J=2.7 Hz, 1H).

### EXAMPLE 47—SYNTHESIS OF N-(5-AMINO-3-CYANOPYRIDIN-2-YL)-N-METHYLACETAMIDE

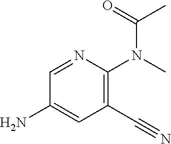

To a stirred solution of N-(3-cyano-5-nitropyridin-2-yl)-N-methylacetamide (700 mg, 3.2 mmol, 1.0 eq.) and Fe (761 mg, 13.6 mmol, 5.0 eq.) in EtOH (10 mL) were added NH₄Cl (850 mg, 16 mmol, 5.0 eq.) and AcOH (1.9 g, 32 mmol, 10 eq.), and the resulting mixture was stirred for 1 h at 80° C. The reaction mixture was then filtered, the solids were washed with EtOH (5×2 mL). The filtrate was concentrated under reduced pressure, and the resulting residue was dissolved in DCM (5 mL). The suspension was filtered, the solids were washed with DCM (5×1 mL), and the filtrate was concentrated under reduced pressure to afford the title compound (600 mg, 99%) as an orange solid. (ES, m/z): [M+H]⁺ 191.1; ¹H NMR (400 MHz, DMSO-d₆): δ 1.74 (s, 3H), 3.08 (s, 3H), 5.98 (d, J=103.2 Hz, 2H), 7.36 (s, 1H), 8.06 (s, 1H).

### EXAMPLE 48—SYNTHESIS OF (Z)-N-HYDROXYTETRAHYDRO-2H-PYRAN-4-CARBIMIDOYL CYANIDE

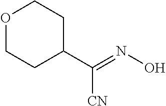 [SMILES: N#C/C(=N\O)C1CCOCC1]

To a stirred mixture of NaOH (7.2 g, 178 mmol, 1.5 eq.) and TMSCN (17.7 g, 178 mmol, 1.5 eq.) in H₂O (150 mL) was added (Z)-N-hydroxytetrahydro-2H-pyran-4-carbimidoyl chloride (14.5 g, 119 mmol, 1.0 eq.) dropwise at 0° C. under nitrogen. The resulting mixture was stirred for 18 h at room temperature under nitrogen. The reaction mixture was then acidified to pH 2 with conc. HCl, and extracted with MTBE (3×200 mL). The combined organic layers were washed with brine (300 mL), dried over anhydrous Na₂SO₄, and concentrated under reduced pressure. This resulted in the title compound, which was used in the next step directly without further purification. (ES, m/z): [M+H]J 155.

### EXAMPLE 49—SYNTHESIS OF (Z)-N-(TOSYLOXY) TETRAHYDRO-2H-PYRAN-4-CARBIMIDOYL CYANIDE

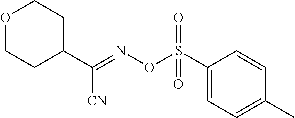 [SMILES: Cc1ccc(S(=O)(=O)O/N=C(\C#N)C2CCOCC2)cc1]

To a stirred mixture of (Z)-N-hydroxytetrahydro-2H-pyran-4-carbimidoyl cyanide (15.0 g, 133 mmol, 1.0 eq.) and TsCl (30.6 g, 160 mmol, 1.2 eq.) in DCM (150 mL) was added TEA (27.0 g, 267 mmol, 2.0 eq.) dropwise at 0° C. under nitrogen. The reaction mixture was stirred for 18 h at room temperature under nitrogen, and was then quenched with water, and extracted with DCM (2×200 mL). The combined organic layers were washed with brine (3×200 mL), dried over anhydrous Na₂SO₄, and concentrated under reduced pressure. This resulted in the title compound, which was used in the next step directly without further purification. (ES, m/z): [M+H]⁺ 309.

### EXAMPLE 50—SYNTHESIS OF METHYL 4-AMINO-3-(OXAN-4-YL)-1,2-THIAZOLE-5-CARBOXYLATE

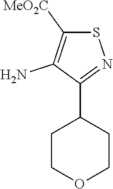 [SMILES: COC(=O)c1snc(C2CCOCC2)c1N]

To a stirred mixture of (Z)-N-(tosyloxy)tetrahydro-2H-pyran-4-carbimidoyl cyanide (1.0 g, 3.2 mmol, 1.0 eq.) and methyl thioglycolate (0.4 g, 3.9 mmol, 1.2 eq.) in MeOH (30 mL) was added TEA (0.66 g, 6.5 mmol, 2.0 eq.) dropwise at 0° C. under nitrogen. The reaction mixture was then quenched with water, and extracted with EtOAc (3×30 mL). The combined organic layers were washed with brine (50 mL), dried over anhydrous Na₂SO₄, and concentrated under reduced pressure. The residue was purified by reversed-phase flash chromatography with the following conditions: column, C18 silica gel; mobile phase, MeCN in Water (0.1% NH₃·H₂O), 10% to 50% gradient in 10 min; detector, UV 254 nm. This resulted in the title compound (250 mg, 31%) as a yellow solid. (ES, m/z): [M+H]⁺ 243.

### EXAMPLE 51—SYNTHESIS OF METHYL 4-IODO-3-ISOPROPYL-1,2-THIAZOLE-5-CARBOXYLATE

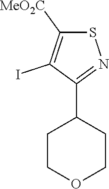 [SMILES: COC(=O)c1snc(C2CCOCC2)c1I]

A mixture of methyl 4-amino-3-(oxan-4-yl)-1,2-thiazole-5-carboxylate (250 mg, 1.0 mmol, 1.0 eq.), isopentyl nitrite (181 mg, 1.5 mmol, 1.5 eq.) and I₂ (1.3 g, 5.2 mmol, 5.0 eq.) in MeOH (10 mL) was stirred for 30 min at 60° C. under nitrogen. The reaction mixture was then quenched with sat. NaHSO₃ (aq.), and extracted with DCM (3×10 mL). The combined organic layers were washed with brine (1×30 mL), dried over anhydrous Na₂SO₄, and concentrated under reduced pressure. The residue was purified by reversed-phase flash chromatography with the following conditions: column, C18 silica gel; mobile phase, MeCN in Water (0.1% NH₃·H₂O), 10% to 50% gradient in 10 min; detector, UV 254 nm. This resulted in the title compound (150 mg, 41%) as a yellow solid. (ES, m/z): [M+H]⁺ 354.

### EXAMPLE 52—SYNTHESIS OF (Z)-N-(TOSYLOXY)ISOBUTYRIMIDOYL CYANIDE

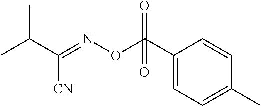

To a stirred mixture of (Z)-N-hydroxyisobutyrimidoyl cyanide (15.0 g, 133 mmol, 1.0 eq.) and TsCl (30.6 g, 160 mmol, 1.2 eq.) in DCM (150 mL) was added TEA (27.0 g, 267 mmol, 2.0 eq.) dropwise at 0° C. under nitrogen. The reaction mixture was stirred for 18 h at room temperature under nitrogen, and was then quenched with water, and extracted with DCM (2×200 mL). The combined organic layers were washed with brine (3×200 mL), dried over anhydrous Na₂SO₄, and concentrated under reduced pressure. This resulted in the title compound, which was used in the next step directly without further purification. (ES, m/z): [M+H]⁺ 267.

### EXAMPLE 53—SYNTHESIS OF METHYL 4-AMINO-3-ISOPROPYL-1,2-THIAZOLE-5-CARBOXYLATE

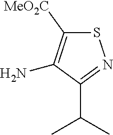

To a stirred mixture of (Z)-N-(tosyloxy)isobutyrimidoyl cyanide (15.0 g, 56 mmol, 1.0 eq.) and methyl thioglycolate (7.2 g, 68 mmol, 1.2 eq.) in MeOH (50 mL) was added TEA (17.1 g, 168 mmol, 3.0 eq.) dropwise at 0° C. under nitrogen. The reaction mixture was stirred for 18 h at room temperature under nitrogen, and was then quenched with water, and extracted with EtOAc (3×50 mL). The combined organic layers were washed with brine (50 mL), dried over Na₂SO₄, and concentrated under reduced pressure. The residue was purified by silica gel column chromatography, eluted with PE/EtOAc (30:1) to afford the title compound (4.5 g, 39%) as a yellow oil. (ES, m/z): [M+H]⁺ 201.

### EXAMPLE 54—SYNTHESIS OF METHYL 4-IODO-3-ISOPROPYL-1,2-THIAZOLE-5-CARBOXYLATE

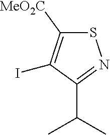

A mixture of methyl 4-amino-3-isopropyl-1,2-thiazole-5-carboxylate (2 g, 10.0 mmol, 1.0 eq.), isopentyl nitrite (1.75 g, 14.9 mmol, 1.5 eq.) and I₂ (12.6 g, 50.0 mmol, 5.0 eq.) in CHCl₃ (50 mL) was stirred for 1 h at 60° C. under nitrogen. The reaction was quenched with sat. NaHSO₃ (aq.) at room temperature, and extracted with DCM (2×50 mL). The combined organic layers were washed with brine (1×50 mL), dried over anhydrous Na₂SO₄, and concentrated under reduced pressure. The residue was purified by reversed-phase flash chromatography with the following conditions: column, C18 silica gel; mobile phase, MeCN in Water (0.1% NH₃·H₂O), 10% to 50% gradient in 10 min; detector, UV 254 nm. This resulted in the title compound (1.5 g, 48%) as a yellow solid. (ES, m/z): [M+H]⁺ 312.

### EXAMPLE 55—SYNTHESIS OF 2-(1-METHYL-6-OXO-1,6-DIHYDROPYRIDIN-3-YL)ACETONITRILE

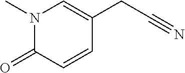

A mixture of 5-bromo-1-methylpyridin-2-one (7.0 g, 37 mmol, 1.0 eq.), sodium 2-cyanoacetate (8.0 g, 75 mmol, 2.0 eq.), SPhos (2.3 g, 5.6 mmol, 0.15 eq.) and Pd₂(allyl)₂Cl₂ (1.4 g, 3.7 mmol, 0.1 eq.) in mesitylene (50 mL) was stirred for 3 h at 140° C. under nitrogen. The resulting mixture was then concentrated under reduced pressure, and the residue was purified by silica gel column chromatography, eluted with PE/EtOAc (1:1) to afford the title compound (2.1 g, 38%) as an off-white solid. (ES, m/z): [M+H]⁺ 149. ¹H NMR (400 MHz, DMSO-d₆): δ 3.41 (s, 3H), 3.76 (d, J 0.9 Hz, 2H), 6.43 (d, J=9.3 Hz, 1H), 7.44-7.38 (m, 1H), 7.70 (d, J=2.7 Hz, 1H).

### EXAMPLE 56—SYNTHESIS OF (E)-N-HYDROXY-1-METHYL-6-OXO-1,6-DIHYDROPYRIDINE-3-CARBIMIDOYL CYANIDE

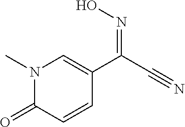

To a stirred mixture of 2-(1-methyl-6-oxo-1,6-dihydropyridin-3-yl)acetonitrile (280 mg, 1.9 mmol, 1.0 eq.) and EtONa (257 mg, 3.8 mmol, 2.0 eq.) in EtOH (6 mL) were added isopentyl nitrite (244 mg, 2.1 mmol, 1.1 eq.) at 0° C. under nitrogen. The resulting mixture was stirred for 2 h at room temperature under nitrogen. The resulting mixture was then concentrated under reduced pressure, and the residue was purified by reverse flash chromatography with the following conditions: column, C18 silica gel; mobile phase, MeCN in Water, 10% to 50% gradient in 10 min; detector, UV 254 nm, to afford the title compound (40 mg, 12%) as an off-white solid. (ES, m/z): [M+H]⁺ 177. ¹H NMR (400 MHz, DMSO-d₆): δ 3.35 (s, 3H), 6.61-6.44 (m, 1H), 7.92-7.84 (m, 1H), 8.65 (d, J=2.7 Hz, 1H), 13.68 (s, 1H).

### EXAMPLE 57—SYNTHESIS OF (Z)-1-METHYL-6-OXO-N-(TOSYLOXY)-1,6-DIHYDROPYRIDINE-3-CARBIMIDOYL CYANIDE

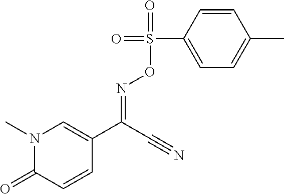

A mixture of (E)-N-hydroxy-1-methyl-6-oxo-1,6-dihydropyridine-3-carbimidoyl cyanide (500 mg, 2.8 mmol, 1.0 eq.) and TsCl (538 mg, 2.8 mmol, 1.0 eq.) in toluene (5 mL) was stirred for 2 h at 110° C. under nitrogen. The resulting mixture was then extracted with EtOAc (3×10 mL). The combined organic layers were washed with dioxane (10 mL), dried over Na₂SO₄, and concentrated under reduced pressure to afford the title compound (400 mg, 43%) as an off-white solid. (ES, m/z): [M+H]⁺ 332.

### EXAMPLE 58—SYNTHESIS OF METHYL 4-AMINO-3-(1-METHYL-6-OXO-1,6-DIHYDROPYRIDIN-3-YL)ISOTHIAZOLE-5-CARBOXYLATE

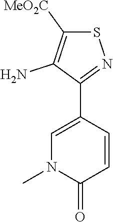 [SMILES: COC(=O)c1snc(-c2ccc(=O)n(C)c2)c1N]

To a stirred mixture of (Z)-1-methyl-6-oxo-N-(tosyloxy)-1,6-dihydropyridine-3-carbimidoyl cyanide (2.1 g, 6.3 mmol, 1.0 eq.) and methyl thioglycolate (0.81 g, 7.6 mmol, 1.2 eq.) in MeOH (20 mL) was added NEt₃ (1.3 g, 12.7 mmol, 2.0 eq.) at room temperature under nitrogen. The resulting mixture was stirred for 3 h at room temperature. The reaction mixture was evaporated to dryness and then poured into water (50 mL) and extracted with EtOAc (3×40 mL). The combined organic phase was washed with brine (50 mL) and dried with Na₂SO₄ and filtered, the filtrate was evaporated to dryness. The residue was purified by silica gel column chromatography, eluted with PE/EtOAc (1:1) to afford the title compound (1.0 g, 59%) as an off-white solid. (ES, m/z): [M+H]⁺ 266. ¹H NMR (400 MHz, DMSO-d₆): δ 3.34 (s, 3H), 3.84 (s, 3H), 6.42 (s, 2H), 6.51 (d, J=9.4 Hz, 1H), 7.79-7.70 (m, 1H), 8.13 (d, J=2.6 Hz, 1H).

### EXAMPLE 59—SYNTHESIS OF METHYL 4-IODO-3-(1-METHYL-6-OXO-1,6-DIHYDROPYRIDIN-3-YL)ISOTHIAZOLE-5-CARBOXYLATE

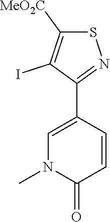 [SMILES: COC(=O)c1snc(-c2ccc(=O)n(C)c2)c1I]

A mixture of 4-amino-3-(1-methyl-6-oxo-1,6-dihydropyridin-3-yl)isothiazole-5-carboxylate (800 mg, 3.0 mmol, 1.0 eq.), isopentyl nitrite (565 mg, 4.8 mmol, 1.6 eq.) and 12 (3.8 g, 15 mmol, 5.0 eq.) in CHCl₃ (7 mL) was stirred for 1 h at 50° C. The resulting mixture was extracted with EtOAc (3×10 mL). The combined organic layers were washed with dioxane (10 mL), dried over anhydrous Na₂SO₄, and concentrated under reduced pressure to afford the title compound (300 mg, 26%) as an off-white solid. (ES, m/z): [M+H]⁺ 376. ¹H NMR (DMSO-d₆, 400 MHz, ppm): δ 3.53 (s, 3H), 3.93 (s, 3H), 6.51 (d, J=9.4 Hz, 1H), 7.83-7.76 (m, 1H), 8.21 (d, J=2.6 Hz, 1H).

### EXAMPLE 60—SYNTHESIS OF (E)-N-HYDROXY-1-METHYL-1H-PYRAZOLE-4-CARBIMIDOYL CYANIDE

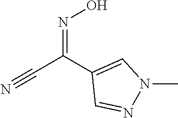

To a stirred solution of 2-(1-methylpyrazol-4-yl) acetonitrile (4.5 g, 37 mmol, 1.0 eq.) and EtONa (7.6 g, 112 mmol, 3.0 eq.) in EtOH (20 mL) was added isopentyl nitrite (6.5 g, 56 mmol, 1.5 eq.) in EtOH (20 mL) dropwise at 0° C. under nitrogen. The resulting mixture was stirred for 4 h at room temperature under nitrogen, and was then diluted with diethyl ether (500 mL). The solids were collected by filtration and washed with diethyl ether (3×10 mL). This resulted in the tiotle compound (4.5 g, 81%) as a pink solid. (ES, m/z): [M+H]⁺ 151.

### EXAMPLE 61—SYNTHESIS OF (Z)-1-METHYL-N-(TOSYLOXY)-1H-PYRAZOLE-4-CARBIMIDOYL CYANIDE

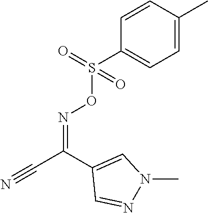 [SMILES: Cc1ccc(S(=O)(=O)O/N=C(/C#N)c2cnn(C)c2)cc1]

A solution of (E)-1-methylpyrazole-4-carbonimidoyl cyanide (4.5 g, 30 mmol, 1.0 eq.) and TsCl (5.7 g, 30 mmol, 1.0 eq.) in DCM (50 mL) was stirred for 1 h at room temperature under nitrogen. The resulting mixture was concentrated under reduced pressure to afford the title compound (4.5 g, 49%) as a light yellow oil. (ES, m/z): [M+H]⁺ 305.

### EXAMPLE 62—SYNTHESIS OF METHYL 4-AMINO-3-(1-METHYL-1H-PYRAZOL-4-YL)ISOTHIAZOLE-5-CARBOXYLATE

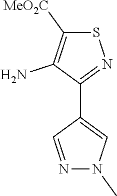 [SMILES: COC(=O)c1snc(-c2cnn(C)c2)c1N]

A solution of (Z)-1-methyl-N-(tosyloxy)-1H-pyrazole-4-carbimidoyl cyanide (4.5 g, 15 mmol, 1.0 eq.), Et₃N (3.0 g, 30 mmol, 2.0 eq.) and methyl thioglycolate (1.9 g, 18 mmol, 1.2 eq.) in MeOH (40 mL) was stirred overnight at 60° C. under nitrogen. The resulting mixture was then concentrated under reduced pressure, and the residue was purified by silica gel column chromatography, eluted with PE/EtOAc (1:1) to afford the title compound (330 mg, 9%) as a light yellow solid. (ES, m/z): [M+H]⁺ 239; ¹H NMR (400 MHz, DMSO-d₆): δ3.83 (s, 3H), 3.91 (s, 3H), 6.19 (s, 2H), 7.94 (s, 1H), 8.30 (s, 1H).

### EXAMPLE 63—SYNTHESIS OF METHYL 4-IODO-3-(1-METHYL-1H-PYRAZOL-4-YL)ISOTHIAZOLE-5-CARBOXYLATE

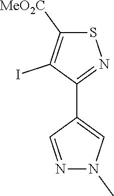 [SMILES: COC(=O)c1snc(-c2cnn(C)c2)c1I]

A solution of methyl 4-amino-3-(1-methyl-1H-pyrazol-4-yl)isothiazole-5-carboxylate (330 mg, 1.4 mmol, 1.0 eq.), isopentyl nitrite (243 mg, 2.1 mmol, 1.5 eq.) and 12 (1.4 g, 5.5 mmol, 4.0 eq.) in CHCl₃ (20 mL) was stirred for 30 min at 50° C. under nitrogen. The mixture was allowed to cool to room temperature, and then was quenched with sat. NaHSO₃ (50 mL) at 0° C. The resulting mixture was extracted with DCM (3×100 mL), and the combined organic layers were washed with brine (100 mL), dried over anhydrous Na₂SO₄, and concentrated under reduced pressure. This resulted in the title compound (400 mg, 83%) as a light yellow oil. (ES, m/z): [M+H]⁺ 350; ¹H NMR (400 MHz, DMSO-d₆): δ 3.91 (s, 3H), 3.94 (s, 3H), 8.06 (d, J=0.8 Hz, 1H), 8.46 (s, 1H).

### EXAMPLE 64—SYNTHESIS OF ETHYL 3-(BENZYLOXY)-4-BROMOISOTHIAZOLE-5-CARBOXYLATE

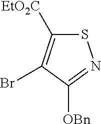 [SMILES: CCOC(=O)c1snc(OCc2ccccc2)c1Br]

To a solution of LDA (2.0 M in THF, 55 mL, 28 mmol, 1.5 eq.) in THF (200 mL) was added 3-(benzyloxy)-4-bromo-1,2-thiazole (5.0 g, 19 mmol, 1.0 eq.), and the reaction mixture was stirred for 1 h at −78° C. under nitrogen. 2-Ethoxy-2-oxoacetonitrile (2.2 g, 22 mmol, 1.2 eq.) was then added dropwise at −78° C., and the mixture was stirred for another 3 h at −60° C. under nitrogen. The reaction was then quenched with sat. NH₄Cl at 0° C., and the resulting mixture was extracted with EtOAc (2×150 mL). The combined organic layers were washed with brine (2×50 mL), dried over anhydrous Na₂SO₄, and concentrated under reduced pressure. The residue was purified by reversed-phase flash chromatography with the following conditions: column, C18 silica gel; mobile phase, MeCN in Water (0.1% FA), 85% gradient in 10 min; detector, UV 254 nm to afford the title compound (2.0 g, 32%) as a brown oil. (ES, m/z): [M+H]⁺ 342.0; ¹H NMR (400 MHz, DMSO-d₆): δ 1.31 (t, J=7.1 Hz, 3H), 4.36 (q, J=7.1 Hz, 2H), 5.48 (s, 2H), 7.34-7.45 (m, 3H), 7.45-7.56 (m, 2H).

### EXAMPLE 65—SYNTHESIS OF ETHYL 3-(BENZYLOXY)-4-(TRIFLUORO-METHYL) ISOTHIAZOLE-5-CARBOXYLATE

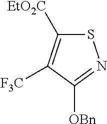 [SMILES: CCOC(=O)c1snc(OCc2ccccc2)c1C(F)(F)F]

A mixture of ethyl 3-(benzyloxy)-4-bromo-1,2-thiazole-5-carboxylate (2 g, 5.8 mmol, 1.0 eq.) and CuI (2.2 g, 11.7 mmol, 2.0 eq.) in DMF (20 mL) was stirred for 4 h at 90° C. under nitrogen. The resulting mixture was filtered, the solids were washed with MeCN (3×10 mL), and the filtrate was concentrated under reduced pressure. Water was added, and the mixture was extracted with EtOAc (2×150 mL). The combined organic layers were washed with brine (2×50 mL), dried over anhydrous Na₂SO₄, and concentrated under reduced pressure. The residue was purified by reversed-phase flash chromatography with the following conditions: column, C18 silica gel; mobile phase, MeCN in Water (0.1% FA), 80% to 90% gradient in 10 min; detector, UV 254 nm to afford the title compound (1.92 g, 99%) as a yellow oil. (ES, m/z): [M+H]⁺ 332.0; ¹H NMR (400 MHz, DMSO-d₆): δ 1.30 (t, J=7.1 Hz, 3H), 4.37 (q, J=7.1 Hz, 2H), 5.50 (s, 2H), 7.35-7.48 (m, 5H).

### EXAMPLE 66—SYNTHESIS OF ETHYL 3-HYDROXY-4-(TRIFLUOROMETHYL) ISOTHIAZOLE-5-CARBOXYLATE

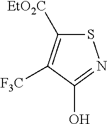 [SMILES: CCOC(=O)c1snc(O)c1C(F)(F)F]

A solution of ethyl 3-(benzyloxy)-4-(trifluoromethyl)-1,2-thiazole-5-carboxylate (2.0 g, 6.0 mmol, 1.0 eq.) and BBr₃ (1 M in DCM, 12 mL, 12 mmol, 2.0 eq.) in DCM (20 mL) was stirred for 2 h at 25° C. under nitrogen. The reaction was quenched with MeOH at 0° C. and then diluted with water at 25° C., and the resulting mixture was extracted with DCM (2×150 mL). The combined organic layers were washed with brine (2×50 mL), dried over anhydrous Na₂SO₄, and concentrated under reduced pressure. The residue was purified by reversed-phase flash chromatography with the following conditions: column, C18 silica gel; mobile phase, Solvent A MeCN in Solvent B H₂O, 50% to 70% gradient in 10 min; detector, UV 254 nm to afford the title compound (1.5 g) as a yellow oil, which was used without further purification. (ES, m/z): [M+H]⁺ 242.0; ¹H NMR (400 MHz, DMSO-d₆): δ 1.30 (t, J=7.1 Hz, 3H), 4.36 (q, J=7.1 Hz, 2H), 13.20 (s, 2H).

### EXAMPLE 67—SYNTHESIS OF ETHYL 4-(TRIFLUOROMETHYL)-3-(((TRIFLUOROMETHYL)SULFONYL)OXY) ISOTHIAZOLE-5-CARBOXYLATE

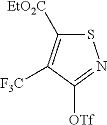 [SMILES: CCOC(=O)c1snc(OS(=O)(=O)C(F)(F)F)c1C(F)(F)F]

To a solution of ethyl 3-hydroxy-4-(trifluoromethyl)-1,2-thiazole-5-carboxylate (1.5 g, 6.2 mmol, 1.0 eq.) and TEA (1.9 g, 19 mmol, 3.0 eq.) in DCM (50 mL) was added Tf₂O (3.5 g, 12 mmol, 2.0 eq.) dropwise over 1 min at 0° C. The resulting mixture was stirred for 2 h at 25° C., and then quenched with water at 25° C., and the resulting mixture was extracted with DCM (2×150 mL). The combined organic layers were washed with brine (2×50 mL), dried over anhydrous Na₂SO₄, and concentrated under reduced pressure. The residue was purified by reversed-phase flash chromatography with the following conditions: column, C18 silica gel; mobile phase, MeCN in Water (0.1% FA), 70% to 80% gradient in 10 min; detector, UV 254 nm to afford the title compound (500 mg, 22%) as a yellow oil. (ES, m/z): [M+H]⁺ 374.0; ¹H NMR (400 MHz, DMSO-d₆): δ 1.32 (t, J=7.2 Hz, 3H), 4.42 (q, J=7.2 Hz, 2H).

### EXAMPLE 68—SYNTHESIS OF METHYL 3-(3-CHLOROPYRIDIN-2-YL)-4-CYCLOPROPYLISOTHIAZOLE-5-CARBOXYLATE

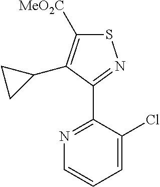 [SMILES: COC(=O)c1snc(-c2ncccc2Cl)c1C1CC1]

To a stirred solution of methyl 3-(3-chloropyridin-2-yl)-4-iodo-1,2-thiazole-5-carboxylate (360 mg, 0.9 mmol, 1.0 eq.) and cyclopropylboronic acid (98 mg, 1.1 mmol, 1.2 eq.) in dioxane (5 mL) were added Pd(PPh₃)₂Cl₂ (66 mg, 0.1 mmol, 0.1 eq.) and Ag₂O (438 mg, 1.9 mmol, 2.0 eq.) at room temperature under nitrogen. The solution was stirred at 90° C. for 16 h, and then cooled and diluted with water (20 mL). The resulting mixture was extracted with EtOAc (3×50 mL). The combined organic layers were washed with brine (50 mL), dried over Na₂SO₄, and concentrated under reduced pressure. The residue was purified by Prep-TLC (PE/EtOAc=3/1) to afford the title compound (110 mg, 39%) as a yellow oil. (ES, m/z): [M+H]⁺ 295.

### EXAMPLE 69—SYNTHESIS OF 3-(3-CHLOROPYRIDIN-2-YL)-4-CYCLOPROPYL-ISOTHIAZOLE-5-CARBOXYLIC ACID

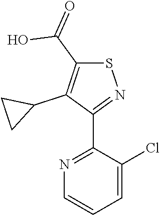 [SMILES: O=C(O)c1snc(-c2ncccc2Cl)c1C1CC1]

General procedure B (using methyl 3-(3-chloropyridin-2-yl)-4-cyclopropyl-1,2-thiazole-5-carboxylate) yielded the title compound (90 mg, 94%) as a yellow solid. (ES, m/z): [M+H]⁺ 281.

### EXAMPLE 70—SYNTHESIS OF METHYL 5-PHENYLISOTHIAZOLE-3-CARBOXYLATE

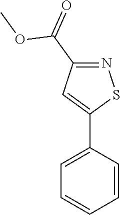

A mixture of methyl 2-imino-4-oxo-4-phenylbutanoate (1.1 g, 5.4 mmol, 1.0 eq.) and P₂S₅(1.2 g, 5.4 mmol, 1.0 eq.) in THF (3 mL) was stirred for 2 h at room temperature under nitrogen. The resulting mixture was then concentrated under reduced pressure, and was diluted with EtOAc (3 mL). To the above mixture was added H₂O₂(30%) (0.22 g, 6.43 mmol, 1.2 eq.) dropwise at 0° C. The resulting mixture was stirred for 2 h at room temperature. The reaction was quenched with water at room temperature. The resulting mixture was extracted with EtOAc (3×10 mL). The combined organic layers were washed with brine (5 mL), dried over Na₂SO₄ and concentrated under reduced pressure. The residue was purified by reversed-phase flash chromatography with the following conditions: column, C18 silica gel; mobile phase, MeCN in water (0.1% NH₃·H₂O), 10% to 50% gradient in 10 min; detector, UV 254 nm, affording the title compound (250 mg, 21%) as a yellow solid. (ES, m/z): [M+H]⁺ 220.

### EXAMPLE 71—SYNTHESIS OF METHYL 4-IODO-5-PHENYLISOTHIAZOLE-3-CARBOXYLATE

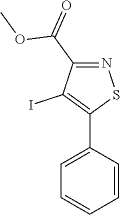 [SMILES: COC(=O)c1nsc(-c2ccccc2)c1I]

A mixture of methyl 5-phenylisothiazole-3-carboxylate (1.0 g, 4.6 mmol, 1.0 eq.), I₂ (2.3 g, 9.1 mmol, 2.0 eq.) and HNO₃ (25 mL) in MeCN (50 mL) was stirred overnight at 80° C. under nitrogen. The reaction was quenched with sat. NaHSO₃ (aq.) at room temperature, and extracted with EtOAc (3×20 mL). The combined organic layers were washed with brine (30 mL), dried over Na₂SO₄ and concentrated under reduced pressure. The residue was purified by silica gel column chromatography, eluted with PE/EtOAc (12:1) to afford the title compound (300 mg, 19%) as a yellow oil. (ES, m/z): [M+H]⁺ 346.

### EXAMPLE 72—SYNTHESIS OF METHYL 5-PHENYL-4-(TRIFLUOROMETHYL) ISOTHIAZOLE-3-CARBOXYLATE

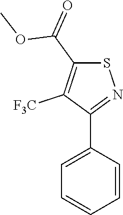 [SMILES: COC(=O)c1snc(-c2ccccc2)c1C(F)(F)F]

A mixture of methyl 4-iodo-5-phenylisothiazole-3-carboxylate (300 mg, 0.87 mmol, 1.0 eq.), methyl 2,2-difluoro-2-sulfoacetate (333 mg, 1.7 mmol, 2.0 eq.) and CuI (331 mg, 1.7 mmol, 2.0 eq.) in DMF (3 mL) was stirred for 2 h at 80° C. under nitrogen. The resulting mixture was then filtered, the filter cake was washed with EtOAc (2×5 mL), and the filtrate was concentrated under reduced pressure. The resulting mixture was extracted with EtOAc (3×5 mL). The combined organic layers were washed with brine (10 mL), dried over anhydrous Na₂SO₄ and concentrated under reduced pressure. The residue was purified by Prep-TLC (PE/EtOAc 6:1) to afford the title compound (210 mg, 84%) as a yellow solid. (ES, m/z): [M+H]⁺ 288.

### EXAMPLE 73—SYNTHESIS OF 5-PHENYL-4-(TRIFLUOROMETHYL) ISOTHIAZOLE-3-CARBOXYLIC ACID

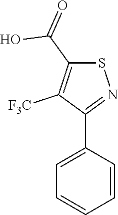 [SMILES: O=C(O)c1snc(-c2ccccc2)c1C(F)(F)F]

General procedure B (using methyl 5-phenyl-4-(trifluoromethyl)isothiazole-3-carboxylate) yielded the title compound (200 mg, crude) as a white solid. (ES, m/z): [M+H]⁺ 274.

### EXAMPLE 74—SYNTHESIS OF (E)-N-HYDROXYBENZO[D]THIAZOLE-7-CARBIMIDOYL CYANIDE

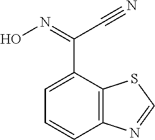 [SMILES: N#C/C(=N/O)c1cccc2ncsc12]

To a stirred solution of 2-(benzo[d]thiazol-7-yl)acetonitrile (46 g, 264 mmol, 1.0 eq.) and NaOH (21.1 g, 528 mmol, 2.0 eq.) in EtOH (280 mL) were added isopentyl nitrite (37.1 g, 317 mmol, 1.2 eq.) dropwise at 0° C. under nitrogen. The resulting mixture was stirred for 1 h at room temperature, and then was diluted with diethyl ether (2.5 L). The solids were collected by filtration and washed with diethyl ether (3×100 mL). This resulted in the title compound (54 g) as a grey solid, which was used without further purification. (ES, m/z): [M+H]⁺ 204.

### EXAMPLE 75—SYNTHESIS OF (E)-N-(TOSYLOXY)BENZO[D]THIAZOLE-7-CARBIMIDOYL CYANIDE

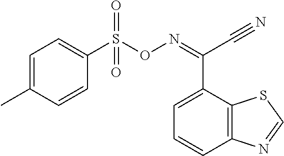 [SMILES: Cc1ccc(S(=O)(=O)O/N=C(/C#N)c2cccc3ncsc23)cc1]

To a stirred solution of (E)-N-hydroxybenzo[d]thiazole-7-carbimidoyl cyanide (54 g, 266 mmol, 1.0 eq.) and TEA (53.8 g, 531 mmol, 2.0 eq.) in DCM (400 mL) were added TsCl (58.3 g, 306 mmol, 1.15 eq.) dropwise at 0° C. under nitrogen atmosphere. The resulting mixture was stirred for 2 h at room temperature under nitrogen. The resulting mixture was then diluted with water (100 mL), and extracted with DCM (3×100 mL). The combined organic layers were washed with brine (1×100 mL), dried over anhydrous Na₂SO₄, and concentrated under reduced pressure. This resulted in the title compound (60 g) as a red solid, which was used without further purification. (ES, m/z): [M+H]⁺ 358.

### EXAMPLE 76—SYNTHESIS OF METHYL 4-AMINO-3-(BENZO[D]THIAZOL-7-YL)ISOTHIAZOLE-5-CARBOXYLATE

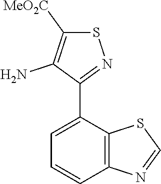 [SMILES: COC(=O)c1snc(-c2cccc3ncsc23)c1N]

To a stirred solution of (E)-N-(tosyloxy)benzo[d]thiazole-7-carbimidoyl cyanide (60 g, 168 mmol, 1.0 eq.) and methyl 2-mercaptoacetate (23.2 g, 218 mmol, 1.3 eq.) in MeOH (50 mL) was added TEA (34.0 g, 336 mmol, 2.0 eq.) dropwise at 0° C. under nitrogen. The resulting mixture was stirred for 2 h at room temperature, and then diluted with water (20 mL), and extracted with EtOAc (3×20 mL). The combined organic layers were washed with brine (10 mL), dried over Na₂SO₄, and concentrated under reduced pressure. The residue was purified by silica gel column chromatography, eluted with PE/EtOAc (5:1) to afford the title compound (6.0 g, 12%) as a yellow solid. (ES, m/z): [M+H]⁺ 292.

### EXAMPLE 77—SYNTHESIS OF METHYL 3-(BENZO[D]THIAZOL-7-YL)-4-IODOISOTHIAZOLE-5-CARBOXYLATE

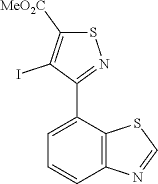 [SMILES: COC(=O)c1snc(-c2cccc3ncsc23)c1I]

A solution of methyl methyl 4-amino-3-(benzo[d]thiazol-7-yl)isothiazole-5-carboxylate (6.0 g, 20.6 mmol, 1.0 eq.), I₂ (26 g, 103 mmol, 5.0 eq.) and isopentyl nitrite (3.6 g, 31 mmol, 1.5 eq.) in CHCl₃ (2 mL) was stirred for 1 h at room temperature under nitrogen. The resulting mixture was stirred for 1 h at 60° C. under nitrogen atmosphere. The resulting mixture was diluted with water (10 mL). The resulting mixture was extracted with DCM (3×10 mL). The combined organic layers were washed with brine (10 mL), dried over anhydrous Na₂SO₄, and concentrated under reduced pressure. The residue was purified by silica gel column chromatography, eluted with PE/EA (12:1) to afford the title compound (2.8 g, 34%) as a light yellow solid. (ES, m/z): [M+H]⁺ 403; ¹H NMR (400 MHz, DMSO-d₆): δ 3.95 (s, 3H), 7.75 (t, J=7.6 Hz, 1H), 7.91-7.96 (m, 1H), 8.26 (dd, J=8.0, 1.2 Hz, 1H), 9.48 (s, 1H).

### EXAMPLE 78—SYNTHESIS OF METHYL 3-(BENZO[D]THIAZOL-7-YL)-4-CYCLOPROPYLISOTHIAZOLE-5-CARBOXYLATE

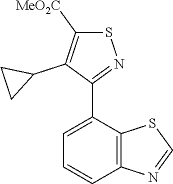 [SMILES: COC(=O)c1snc(-c2cccc3ncsc23)c1C1CC1]

A solution of methyl 3-(1,3-benzothiazol-7-yl)-4-iodo-1,2-thiazole-5-carboxylate (2.8 g, 7.0 mmol, 1.0 eq.), Pd(PPh₃)₂Cl₂ (1.0 g, 1.4 mmol, 0.2 eq.), cyclopropylboronic acid (0.9 g, 10 mmol, 1.5 eq.) and Ag₂O (3.2 g, 14 mmol, 2.0 eq.) in dioxane (30 mL) was stirred for 2 h at 90° C. under nitrogen. The reaction mixture was evaporated to dryness and then poured into water (100 mL) and extracted with EA (3×60 mL). The combined organic phase was washed with brine (100 mL), dried with Na₂SO₄, concentrated under reduced pressure, and purified by silica gel column chromatography, eluted with PE/EtOAc (5:1) to afford the title compound (1.4 g, 64%) as a light yellow solid. (ES, m/z): [M+H]⁺ 317; ¹H NMR (400 MHz, DMSO-d₆): δ 0.22-0.30 (m, 2H), 0.84-0.96 (m, 2H), 3.94 (s, 3H), 7.72 (t, J=7.6 Hz, 1H), 8.09 (d, J=7.6 Hz, 1H), 8.23 (dd, J=8.0, 1.2 Hz, 1H), 9.47 (s, 1H).

### EXAMPLE 79—SYNTHESIS OF 3-(BENZO[D]THIAZOL-7-YL)-4-CYCLOPROPYL-ISOTHIAZOLE-5-CARBOXYLIC ACID

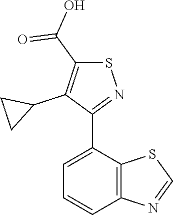 [SMILES: O=C(O)c1snc(-c2cccc3ncsc23)c1C1CC1]

General procedure B (using methyl 3-(1,3-benzothiazol-7-yl)-4-cyclopropyl-1,2-thiazole-5-carboxylate) yielded the title compound (80 mg) as a white solid, which was used without further purification. (ES, m/z): [M+H]⁺ 303.

### EXAMPLE 80—SYNTHESIS OF ETHYL 3-(2-METHOXYQUINOLIN-5-YL)-4-(TRIFLUOROMETHYL) ISOTHIAZOLE-5-CARBOXYLATE

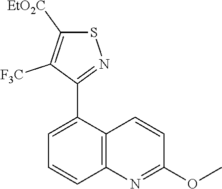 [SMILES: CCOC(=O)c1snc(-c2cccc3nc(OC)ccc23)c1C(F)(F)F]

A mixture of 2-methoxy-5-(4,4,5,5-tetramethyl-1,3,2-dioxaborolan-2-yl)quinoline (115 mg, 0.4 mmol, 1.5 eq.), ethyl 4-(trifluoromethyl)-3-(((trifluoromethyl)sulfonyl)oxy) isothiazole-5-carboxylate (100 mg, 0.3 mmol, 1.0 eq.), K₃PO₄ (170 mg, 0.8 mmol, 3.0 eq.), XPhos (13 mg, 0.03 mmol, 0.1 eq.) and XPhos Pd G3 (22 mg, 0.03 mmol, 0.1 eq.) in toluene (2 mL) was stirred for 18 h at 80° C. under nitrogen. The resulting mixture was then poured into water and extracted with EtOAc (2×10 mL). The combined organic layers were washed with brine (2×5 mL), dried over MgSO₄, and concentrated under reduced pressure. The residue was purified by reversed-phase flash chromatography with the following conditions: column, C18 silica gel; mobile phase, MeCN in water (0.1% FA), 70% to 80% gradient in 10 min; detector, UV 254 nm. This resulted in the title compound (100 mg, 98%) as a yellow oil. (ES, m/z): [M+H]⁺ 383; ¹H NMR (400 MHz, DMSO-d₆): δ1.36 (t, J=7.2 Hz, 3H), 4.02 (s, 3H), 4.45 (q, J=7.2 Hz, 2H), 7.03 (d, J=9.2 Hz, 1H), 7.48 (dd, J=7.2, 1.2 Hz, 1H), 7.71-7.85 (m, 2H), 7.89-7.98 (m, 1H).

### EXAMPLE 81—SYNTHESIS OF 1-METHOXY-5-(4,4,5,5-TETRAMETHYL-1,3,2-DIOXABOROLAN-2-YL)ISOQUINOLINE

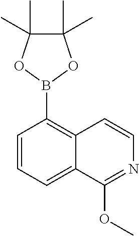

To a stirred mixture of 5-bromo-1-methoxyisoquinoline (4 g, 17 mmol, 1.0 eq.) and bis(pinacolato)diboron (8.5 g, 34 mmol, 2.0 eq.) in DMSO (100 mL) was added KOAc (3.3 g, 34 mmol, 2.0 eq.) and Pd(dppf)Cl₂ (1.9 g, 3.4 mmol, 0.2 eq.) in portions at room temperature under nitrogen atmosphere. The resulting mixture was stirred for 2 h at 80° C., and then was allowed to cool to room temperature and quenched with water, and extracted with EtOAc (2×150 mL). The combined organic layers were washed with brine (2×50 mL), dried over anhydrous Na₂SO₄, and concentrated under reduced pressure. The crude product was washed with hexane (2×30 mL), filtered, and the filtrate was concentrated under reduced pressure. This resulted in the title compound (5.6 g, 93%) as a white solid. (ES, m/z): [M+H]⁺ 286; ¹H NMR (400 MHz, CDCl₃): δ 1.41 (s, 12H), 4.15 (s, 3H), 7.53 (dd, J=8.3, 6.9 Hz, 1H), 8.04 (d, J=6.1 Hz, 1H), 8.13 (dd, J=6.1, 0.9 Hz, 1H), 8.23 (dd, J=6.9, 1.4 Hz, 1H), 8.38 (dt, J=8.3, 1.2 Hz, 1H).

### EXAMPLE 82—SYNTHESIS OF ETHYL 3-(1-METHOXYISOQUINOLIN-5-YL)-4-(TRIFLUOROMETHYL)ISOTHIAZOLE-5-CARBOXYLATE

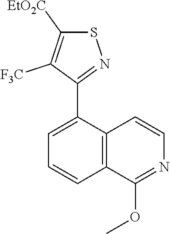 [SMILES: CCOC(=O)c1snc(-c2cccc3c(OC)nccc23)c1C(F)(F)F]

To a stirred mixture of 1-methoxy-5-(4,4,5,5-tetramethyl-1,3,2-dioxaborolan-2-yl) isoquinoline (306 mg, 1.1 mmol, 2.0 eq.) and ethyl 3-(trifluoromethanesulfonyloxy)-4-(trifluoromethyl)-1,2-thiazole-5-carboxylate (200 mg, 0.5 mmol, 1.0 eq.) in toluene (5 mL) were added XPhos (51 mg, 0.1 mmol, 0.2 eq.) and XPhos Pd G3 (45 mg, 0.05 mmol, 0.1 eq.) and K₃PO₄ (284 mg, 1.3 mmol, 2.5 eq.) at room temperature under nitrogen. The resulting mixture was stirred for 3 h at 60° C., and then was allowed to cool to room temperature and quenched with water, and extracted with EtOAc (2×50 mL). The combined organic layers were washed with brine (2×20 mL), dried over anhydrous Na₂SO₄, and concentrated under reduced pressure. The residue was purified by reversed-phase flash chromatography with the following conditions: column, C18 silica gel; mobile phase, MeCN in Water (0.1% NH₃·H₂O), 10% to 50% gradient in 10 min; detector, UV 254 nm. This resulted in the title compound (60 mg, 29%) as a yellow solid. (ES, m/z): [M+H]⁺ 383.

### EXAMPLE 83—SYNTHESIS OF ETHYL 3-(BENZYLOXY)-4-CYCLOPROPYL-ISOTHIAZOLE-5-CARBOXYLATE

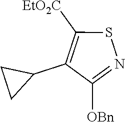 [SMILES: CCOC(=O)c1snc(OCc2ccccc2)c1C1CC1]

A mixture of ethyl 3-(benzyloxy)-4-bromo-1,2-thiazole-5-carboxylate (13.8 g, 40 mmol, 1.0 eq.), cyclopropylboronic acid (5.2 g, 60 mmol, 1.5 eq.), Ag₂O (21.4 g, 93 mmol, 2.3 eq.) and Pd(PPh₃)₂Cl₂ (2.8 g, 4.0 mmol, 0.1 eq.) in dioxane (150 mL) was stirred overnight at 90° C. under nitrogen. The resulting mixture was filtered, the solids were washed with EtOAc (3×50 mL), and the filtrate was diluted with water (300 mL) and extracted with EtOAc (3×100 mL). The combined organic layers were washed with brine (200 mL), dried over Na₂SO₄, and concentrated under reduced pressure. The residue was purified by silica gel column chromatography, eluted with PE/EtOAc (50:1) to afford the title compound (9.5 g, 77%) as a yellow solid. ES, m/z): [M+H]⁺ 304; ¹H NMR (400 MHz, CDCl₃): δ 0.81-0.93 (m, 2H), 1.28-1.34 (m, 2H), 1.38 (t, J=7.2 Hz, 3H), 2.65-2.75 (m, 1H), 4.37 (q, J=7.2 Hz, 2H), 5.38 (s, 2H), 7.29-7.37 (m, 1H), 7.37-7.44 (m, 4H).

### EXAMPLE 84—SYNTHESIS OF ETHYL 4-CYCLOPROPYL-3-HYDROXY-ISOTHIAZOLE-5-CARBOXYLATE

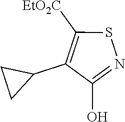 [SMILES: CCOC(=O)c1snc(O)c1C1CC1]

A mixture of ethyl 3-(benzyloxy)-4-cyclopropyl-1,2-thiazole-5-carboxylate (1 g, 3.3 mmol, 1.0 eq.) in HBr in AcOH (10 mL) was stirred for 1 h at room temperature under nitrogen. The reaction was then quenched with water at room temperature, and extracted with EtOAc (3×50 mL). The combined organic layers were washed with brine (20 mL), dried over Na₂SO₄, and concentrated under reduced pressure. The residue was purified by reversed-phase flash chromatography with the following conditions: column, C18 silica gel; mobile phase, MeCN in Water (0.1% TFA), 10% to 50% gradient in 10 min; detector, UV 254 nm. This resulted in the title compound (600 mg, 85%) as a white solid. (ES, m/z): [M+H]+ 214; ¹H NMR (400 MHz, CDCl₃): δ 0.83-0.95 (m, 2H), 1.26-1.36 (m, 2H), 1.41 (t, J=7.2 Hz, 3H), 2.68-2.73 (m, 1H), 4.35 (q, J=7.2 Hz, 2H).

### EXAMPLE 85—SYNTHESIS OF ETHYL 4-CYCLOPROPYL-3-(((TRIFLUORO-METHYL)SULFONYL)OXY) ISOTHIAZOLE-5-CARBOXYLATE

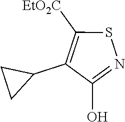 [SMILES: CCOC(=O)c1snc(O)c1C1CC1]

To a stirred mixture of ethyl 4-cyclopropyl-3-hydroxy-1,2-thiazole-5-carboxylate (550 mg, 2.6 mmol, 1.0 eq.) and TEA (782 mg, 7.7 mmol, 3.0 eq.) in DCM (5 mL) was added Tf₂O (1.5 g, 5.2 mmol, 2.0 eq.) dropwise at 0° C. under nitrogen. The resulting mixture was stirred for 2 h at room temperature, and then quenched with water and extracted with EtOAc (3×50 mL). The combined organic layers were washed with brine (20 mL), dried over Na₂SO₄, and concentrated under reduced pressure. The residue was purified by reverse phase flash chromatography with the following conditions: column, C18 silica gel; mobile phase, MeCN in Water (10 mmol/L NH₄HCO₃), 10% to 50% gradient in 10 min; detector, UV 254 nm to afford the title compound (800 mg, 89%) as a colorless oil. (ES, m/z): [M+H]⁺ 346; ¹H NMR (400 MHz, CDCl₃): δ 0.82-0.93 (m, 2H), 1.25-1.34 (m, 2H), 1.40 (t, J=7.2 Hz, 3H), 2.60-2.70 (m, 1H), 4.40 (q, J=7.2 Hz, 2H).

### EXAMPLE 86—SYNTHESIS OF ETHYL 4-CYCLOPROPYL-3-(PYRAZOLO[1,5-A]PYRIDIN-4-YL)ISOTHIAZOLE-5-CARBOXYLATE

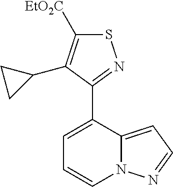 [SMILES: CCOC(=O)c1snc(-c2cccn3nccc23)c1C1CC1]

General procedure F (using 4-(4,4,5,5-tetramethyl-1,3,2-dioxaborolan-2-yl)pyrazolo[1,5-a]pyridine and 4-(4,4,5,5-tetramethyl-1,3,2-dioxaborolan-2-yl)pyrazolo[1,5-a]pyridine) yielded the title compound (80 mg, 66%) as a white solid. (ES, m/z): [M+H]⁺ 314.

### EXAMPLE 87—SYNTHESIS OF ETHYL 4-CYCLOPROPYL-3-(1,4-DIMETHYL-1H-PYRAZOL-3-YL)ISOTHIAZOLE-5-CARBOXYLATE

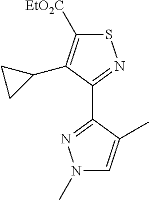 [SMILES: CCOC(=O)c1snc(-c2nn(C)cc2C)c1C1CC1]

General procedure F (using 1,4-dimethyl-3-(4,4,5,5-tetramethyl-1,3,2-dioxaborolan-2-yl)-1H-pyrazole and ethyl 4-cyclopropyl-3-(((trifluoromethyl)sulfonyl)oxy)isothiazole-5-carboxylate) yielded the title compound (70 mg, 14%) as a yellow solid. (ES, m/z): [M+H]⁺ 292.

### EXAMPLE 88—SYNTHESIS OF 1-METHYL-7-(4,4,5,5-TETRAMETHYL-1,3,2-DIOXABOROLAN-2-YL)-1H-BENZO[D][1,2,3]TRIAZOLE

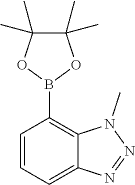 [SMILES: Cn1nnc2cccc(B3OC(C)(C)C(C)(C)O3)c21]

A solution of 7-bromo-1-methyl-1,2,3-benzotriazole (200 mg, 0.9 mmol, 1 eq.) and bis(pinacolato)diboron (480 mg, 1.9 mmol, 2.0 eq.) and Pd(dppf)C₁₂ (138 mg, 0.2 mmol, 0.20 eq.) and AcOK (280 mg, 2.9 mmol, 3.0 eq.) in 1,4-dioxane (10 mL) was stirred overnight at 60° C. under nitrogen. The resulting mixture was diluted with water (10 mL), and extracted with EtOAc (3×50 mL). The combined organic layers were washed with brine (10 mL), dried over Na₂SO₄, and concentrated under reduced pressure. The residue was purified by reversed-phase flash chromatography with the following conditions: column, C18 silica gel; mobile phase, MeCN in Wat, 50% to 70% gradient in 10 min; detector, UV 254 nm, to afford the title compound (130 mg, 53%) as a light yellow oil. (ES, m/z): [M+H]⁺ 260.

### EXAMPLE 89—SYNTHESIS OF ETHYL 4-CYCLOPROPYL-3-(1-METHYL-1H-BENZO[D][1,2,3]TRIAZOL-7-YL)ISOTHIAZOLE-5-CARBOXYLATE

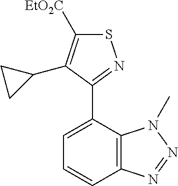 [SMILES: CCOC(=O)c1snc(-c2cccc3nnn(C)c23)c1C1CC1]

General procedure F (using 1-methyl-7-(4,4,5,5-tetramethyl-1,3,2-dioxaborolan-2-yl)-1H-benzo[d][1,2,3]triazole and ethyl 4-cyclopropyl-3-(((trifluoromethyl)sulfonyl)oxy)isothiazole-5-carboxylate) yielded the title compound (100 mg, 61%) as a yellow solid. (ES, m/z): [M+H]⁺ 328.

### EXAMPLE 90—SYNTHESIS OF ETHYL 4-CYCLOPROPYL-3-(2-METHYL-2H-BENZO[D][1,2,3]TRIAZOL-4-YL)ISOTHIAZOLE-5-CARBOXYLATE

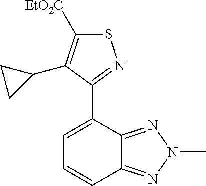 [SMILES: CCOC(=O)c1snc(-c2cccc3nn(C)nc23)c1C1CC1]

General procedure F (using 2-methyl-4-(4,4,5,5-tetramethyl-1,3,2-dioxaborolan-2-yl)-2H-benzo[d][1,2,3]triazole and ethyl 4-cyclopropyl-3-(((trifluoromethyl)sulfonyl)oxy)isothiazole-5-carboxylate) yielded the title compound (90 mg, 63%) as a yellow solid. (ES, m/z): [M+H]⁺ 329.

### EXAMPLE 91—SYNTHESIS OF (1-METHYL-1H-BENZO[D][1,2,3]TRIAZOL-4-YL)BORONIC ACID

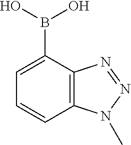

A solution of 4-bromo-1-methyl-1H-benzo[d][1,2,3]triazole (200 mg, 0.9 mmol, 1 eq.) and bis(pinacolato)diboron (480 mg, 1.9 mmol, 2.0 eq.) and Pd(dppf)C₁₂ (138 mg, 0.2 mmol, 0.2 eq.) and AcOK (280 mg, 2.9 mmol, 3.0 eq.) in 1,4-dioxane (10 mL) was stirred overnight at 60° C. under nitrogen. The resulting mixture was diluted with water (10 mL), and extracted with EtOAc (3×50 mL). The combined organic layers were washed with brine (10 mL), dried over Na₂SO₄, and concentrated under reduced pressure. The residue was purified by reversed-phase flash chromatography with the following conditions: column, C18 silica gel; mobile phase, MeCN in Wat, 50% to 70% gradient in 10 min; detector, UV 254 nm, to afford the title compound (90 mg, 54%) as alight yellow oil. (ES, m/z): [M+H]⁺ 178.

### EXAMPLE 92—SYNTHESIS OF ETHYL 4-CYCLOPROPYL-3-(1-METHYL-1H-BENZO[D][1,2,3]TRIAZOL-4-YL)ISOTHIAZOLE-5-CARBOXYLATE

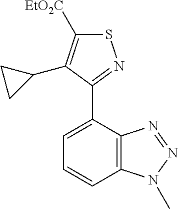 [SMILES: CCOC(=O)c1snc(-c2cccc3c2nnn3C)c1C1CC1]

General procedure F (using (1-methyl-1H-benzo[d][1,2,3]triazol-4-yl)boronic acid and ethyl 4-cyclopropyl-3-(((trifluoromethyl)sulfonyl)oxy)isothiazole-5-carboxylate) yielded the title compound (90 mg, 63%) as a yellow solid. (ES, m/z): [M+H]⁺ 328.

### EXAMPLE 93—SYNTHESIS OF ETHYL 3-(1-(TERT-BUTOXYCARBONYL)-1,2,3,6-TETRAHYDROPYRIDIN-4-YL)-4-CYCLOPROPYLISOTHIAZOLE-5-CARBOXYLATE

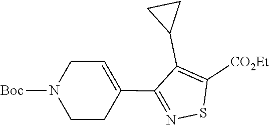 [SMILES: CCOC(=O)c1snc(C2=CCN(C(=O)OC(C)(C)C)CC2)c1C1CC1]

General procedure F (using ethyl 4-cyclopropyl-3-(((trifluoromethyl)sulfonyl)oxy)isothiazole-5-carboxylate and tert-butyl 4-(4,4,5,5-tetramethyl-1,3,2-dioxaborolan-2-yl)-3,6-dihydropyridine-1(2H)-carboxylate) yielded the title compound (250 mg, 52%) as a yellow solid. (ES, m/z): [M+H]⁺ 379.

### EXAMPLE 94—SYNTHESIS OF ETHYL 3-(1-(TERT-BUTOXYCARBONYL)PIPERIDIN-4-YL)-4-CYCLOPROPYLISOTHIAZOLE-5-CARBOXYLATE

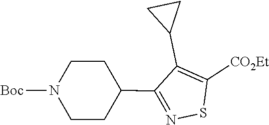 [SMILES: CCOC(=O)c1snc(C2CCN(C(=O)OC(C)(C)C)CC2)c1C1CC1]

A suspension of ethyl 3-(1-(tert-butoxycarbonyl)-1,2,3,6-tetrahydropyridin-4-yl)-4-cyclopropylisothiazole-5-carboxylate (250 mg, 0.7 mmol, 1 eq.), Pd/C (10%, 25 mg) in MeOH (5 mL) was stirred at room temperature overnight under hydrogen. The mixture was then filtered through Celite and concentrated under reduced pressure. This resulted in the title compound (190 mg, 70%) as a brown solid. (ES, m/z): [M+H]⁺ 381.

### EXAMPLE 95—SYNTHESIS OF TERT-BUTYL 4-(5-((5-CYANO-6-(2H-1,2,3-TRIAZOL-2-YL)PYRIDIN-3-YL)CARBAMOYL)-4-CYCLOPROPYLISOTHIAZOL-3-YL)PIPERIDINE-1-CARBOXYLATE

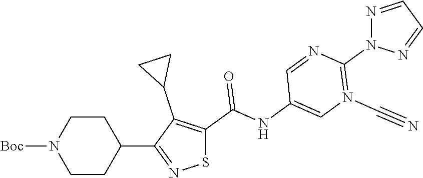

General procedure D (using ethyl 3-(1-(tert-butoxycarbonyl)piperidin-4-yl)-4-cyclopropyl isothiazole-5-carboxylate and 5-amino-2-(2H-1,2,3-triazol-2-yl)nicotinonitrile) yielded the title compound (130 mg, 46%) as a off-white solid. (ES, m/z): [M+H]⁺ 521.

### EXAMPLE 96—SYNTHESIS OF N-(5-CYANO-6-(2H-1,2,3-TRIAZOL-2-YL)PYRIDIN-3-YL)-4-CYCLOPROPYL-3-(PIPERIDIN-4-YL)ISOTHIAZOLE-5-CARBOXAMIDE

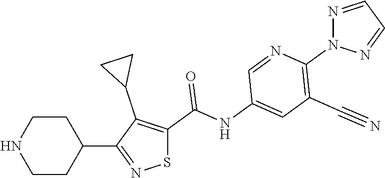 [SMILES: N#Cc1cc(NC(=O)c2snc(C3CCNCC3)c2C2CC2)cnc1-n1nccn1]

The resulting mixture of tert-butyl 4-(5-((5-cyano-6-(2H-1,2,3-triazol-2-yl)pyridin-3-yl)carbamoyl)-4-cyclopropylisothiazol-3-yl)piperidine-1-carboxylate (130 mg, 0.250 mmol, 1 eq.), TFA (1.5 mL) in DCM (4.5 mL) was stirred for 4 h at room temperature under nitrogen. The resulting mixture was then diluted with water (7 mL), and extracted with DCM (3×15 mL). The combined organic layers were washed with brine (7 mL), dried over Na₂SO₄, and concentrated under reduced pressure. The residue was purified by Prep-TLC (PE/EtOAc 1:1) to afford the title compound (80 mg, 69%) as a off-white solid. (ES, m/z): [M+H]⁺ 421.

### EXAMPLE 97—SYNTHESIS OF ETHYL 4-CYCLOPROPYL-3-(2,5-DIHYDRO-FURAN-3-YL)ISOTHIAZOLE-5-CARBOXYLATE

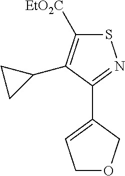 [SMILES: CCOC(=O)c1snc(C2=CCOC2)c1C1CC1]

General procedure F (using ethyl 4-cyclopropyl-3-(trifluoromethanesulfonyloxy)-1,2-thiazole-5-carboxylate and 2-(2,5-dihydrofuran-3-yl)-4,4,5,5-tetramethyl-1,3,2-dioxaborolane) yielded the title compound (170 mg, 88%) as a yellow solid. (ES, m/z): [M+H]⁺ 266.

### EXAMPLE 98—SYNTHESIS OF ETHYL 4-CYCLOPROPYL-3-(TETRAHYDRO-FURAN-3-YL)ISOTHIAZOLE-5-CARBOXYLATE

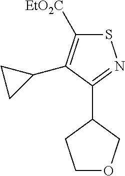 [SMILES: CCOC(=O)c1snc(C2CCOC2)c1C1CC1]

A suspension of ethyl 4-cyclopropyl-3-(2,5-dihydrofuran-3-yl)-1,2-thiazole-5-carboxylate (170 mg, 0.6 mmol, 1.0 eq.) and Pd/C (13 mg, 0.13 mmol, 0.2 eq.) in EtOH (3 mL) was stirred overnight at room temperature under hydrogen. The resulting mixture was filtered, the filter cake was washed with EtOAc (3×3 mL), and the filtrate was concentrated under reduced pressure. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm. This resulted in the title compound (120 mg, 70%) as a yellow solid. (ES, m/z): [M+H]⁺ 268.

### EXAMPLE 99—SYNTHESIS OF 4-CYCLOPROPYL-3-(5,6-DIHYDRO-2H-PYRAN-3-YL)-N-ETHYL-1,2-THIAZOLE-5-CARBOXAMIDE

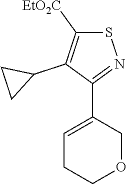 [SMILES: CCOC(=O)c1snc(C2=CCCOC2)c1C1CC1]

General procedure F (using ethyl 4-cyclopropyl-3-(trifluoromethanesulfonyloxy)-1,2-thiazole-5-carboxylate and 2-(5,6-dihydro-2H-pyran-3-yl)-4,4,5,5-tetramethyl-1,3,2-dioxaborolane) yielded the title compound (130 mg, 64%) as a yellow solid. (ES, m/z): [M+H]⁺ 280.

### EXAMPLE 100—SYNTHESIS OF ETHYL 4-CYCLOPROPYL-3-(OXAN-3-YL)-1,2-THIAZOLE-5-CARBOXYLATE

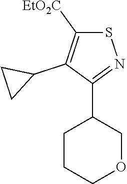 [SMILES: CCOC(=O)c1snc(C2CCCOC2)c1C1CC1]

A suspension of ethyl 4-cyclopropyl-3-(5,6-dihydro-2H-pyran-3-yl)-1,2-thiazole-5-carboxylate (170 mg, 0.6 mmol, 1 eq.) and Pd/C (12 mg, 0.122 mmol, 0.2 eq.) in EtOH (3 mL) was stirred overnight at room temperature under hydrogen. The resulting mixture was filtered, the filter cake was washed with EtOAc (3×3 mL), and the filtrate was concentrated under reduced pressure. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm. This resulted in the title compound (120 mg, 70%) as a yellow solid. (ES, m/z): [M+H]⁺ 282.

### EXAMPLE 101—SYNTHESIS OF ETHYL 3-(1-METHYL-2-OXO-1,2-DIHYDRO-PYRIDIN-3-YL)-4-(TRIFLUOROMETHYL)ISOTHIAZOLE-5-CARBOXYLATE

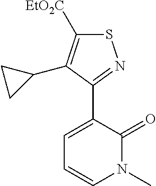 [SMILES: CCOC(=O)c1snc(-c2cccn(C)c2=O)c1C1CC1]

General procedure F (using ethyl 4-cyclopropyl-3-(trifluoromethanesulfonyloxy)-1,2-thiazole-5-carboxylate and 1-methyl-2-oxopyridin-3-yl-boronic acid) yielded the title compound (80 mg, 30%) as a yellow solid. (ES, m/z): [M+H]⁺ 333.

### EXAMPLE 102—SYNTHESIS OF 3-(1-METHYL-2-OXO-1,2-DIHYDROPYRIDIN-3-YL)-4-(TRIFLUOROMETHYL)ISOTHIAZOLE-5-CARBOXYLIC ACID

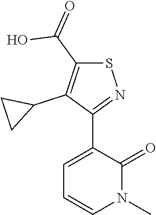 [SMILES: Cn1cccc(-c2nsc(C(=O)O)c2C2CC2)c1=O]

General procedure B (using ethyl 3-(1-methyl-2-oxo-1,2-dihydropyridin-3-yl)-4-(trifluoromethyl)isothiazole-5-carboxylate) yielded the title compound (55 mg, 67%) as a yellow solid. (ES, m/z): [M+H]⁺ 305.

### EXAMPLE 103—SYNTHESIS OF ETHYL 4-CYCLOPROPYL-3-(1-METHYL-1H-INDAZOL-7-YL)ISOTHIAZOLE-5-CARBOXYLATE

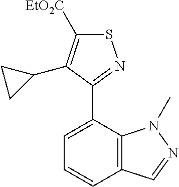 [SMILES: CCOC(=O)c1snc(-c2cccc3cnn(C)c23)c1C1CC1]

A mixture of ethyl 4-cyclopropyl-3-(((trifluoromethyl)sulfonyl)oxy)isothiazole-5-carboxylate (250 mg, 0.7 mmol, 1.0 eq.), 2-(2,5-dihydrofuran-3-yl)-4,4,5,5-tetramethyl-1,3,2-dioxaborolane (170 mg, 0.9 mmol, 1.2 eq.), XPhos Pd G3 (61 mg, 0.07 mmol, 0.1 eq.), XPhos (69 mg, 0.15 mmol, 0.2 eq.) and K₃PO₄ (461 mg, 2.2 mmol, 3.0 eq.) in dioxane (2 mL) was stirred for 2 h at 60° C. under nitrogen. The reaction mixture was then quenched with sat. NH₄Cl (aq.) at room temperature, and extracted with EtOAc (3×20 mL). The combined organic layers were washed with brine (10 mL), dried over Na₂SO₄, and concentrated under reduced pressure. The residue was purified by reverse phase flash chromatography with the following conditions: column, C18 silica gel; mobile phase, MeCN in Water (0.1% TFA), 10% to 50% gradient in 10 min; detector, UV 254 nm to afford the title compound (170 mg, 88%) as a yellow solid. (ES, m/z): [M+H]⁺ 328.

### EXAMPLE 104—SYNTHESIS OF ETHYL 3-(DIMETHYLAMINO)-4-(TRIFLUORO-METHYL)ISOTHIAZOLE-5-CARBOXYLATE

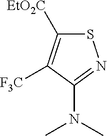 [SMILES: CCOC(=O)c1snc(N(C)C)c1C(F)(F)F]

A mixture of ethyl 4-(trifluoromethyl)-3-(((trifluoromethyl)sulfonyl)oxy)isothiazole-5-carboxylate (100 mg, 0.3 mmol, 1.0 eq.) and TEA (81 mg, 0.8 mmol, 3.0 eq.) in THF (5 mL) and dimethylamine in THF (2 M, 0.12 mL, 0.2 mmol, 1.2 eq.) was stirred for 2 h at 25° C. under nitrogen. The reaction mixture was then quenched with sat. NH₄Cl (aq.) at 0° C., and extracted with EtOAc (2×10 mL). The combined organic layers were washed with brine (5 mL), dried over Na₂SO₄, and concentrated under reduced pressure. The residue was purified by reverse phase flash chromatography with the following conditions: column, C18 silica gel; mobile phase, MeCN in Water (0.1% FA), 60% gradient in 10 min; detector, UV 254 nm to afford the title compound (20 mg, 28%) as a yellow oil. (ES, m/z): [M+H]⁺ 269.

### EXAMPLE 105—SYNTHESIS OF METHYL 4-CYCLOPROPYL-3-ISOPROPYL-ISOTHIAZOLE-5-CARBOXYLATE

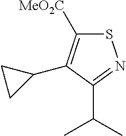

A solution of methyl 4-iodo-3-isopropyl-1,2-thiazole-5-carboxylate (1.0 g, 3.21 mmol, 1.0 eq.), Pd(dppf)Cl₂CH₂Cl₂ (0.5 g, 0.6 mmol, 0.2 eq.), K₂CO₃ (0.9 g, 6.4 mmol, 2.0 eq.) and cyclopropylboronic acid (0.3 g, 3.9 mmol, 1.2 eq.) in dioxane (10 mL) and H₂O (2 mL) was stirred for 2 h at 100° C. under nitrogen. The mixture was allowed to cool down to room temperature, and acidified to pH 5 with 1 M HCl, and extracted with EtOAc (3×10 mL). The combined organic layers were washed with brine (10 mL), dried over Na₂SO₄, and concentrated under reduced pressure. The residue was purified by reverse phase flash chromatography with the following conditions: column, C18 silica gel; mobile phase, MeCN in Water, 30% to 40% gradient in 10 min; detector, UV 254 nm. This resulted in the title compound (200 mg, 28%) as a light yellow oil. (ES, m/z): [M+H]⁺ 226.

### EXAMPLE 106—SYNTHESIS OF 4-CYCLOPROPYL-3-ISOPROPYLISOTHIAZOLE-5-CARBOXYLIC ACID

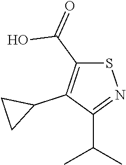

General procedure B (using methyl 4-cyclopropyl-3-isopropyl-1,2-thiazole-5-carboxylate) yielded the title compound (130 mg, 60%) as a yellow solid. (ES, m/z): [M+H]⁺ 212; ¹H NMR (400 MHz, CDCl₃): δ 0.60-0.69 (m, 2H), 0.93-1.03 (m, 2H), 1.25 (d, J=6.8 Hz, 6H), 1.80-1.90 (m, 1H), 3.38-3.58 (m, 1H), 13.67 (s, 1H).

### EXAMPLE 107—SYNTHESIS OF METHYL 4-(3-HYDROXYOXETAN-3-YL)-3-PHENYLISOTHIAZOLE-5-CARBOXYLATE

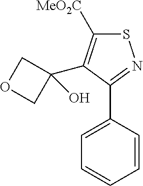 [SMILES: COC(=O)c1snc(-c2ccccc2)c1C1(O)COC1]

A solution of methyl 4-iodo-3-phenylisothiazole-5-carboxylate (600 mg, 1.7 mmol, 1.0 eq.) in THF (10 mL) was treated with iPrMgCl—LiCl (1.3 M in THF, 4.0 mL, 5.2 mmol, 3.0 eq.) for 30 min at −40° C. under nitrogen, followed by the addition of 3-oxetanone (190 mg, 2.6 mmol, 1.5 eq.) dropwise at −40° C. The resulting mixture was stirred for 1 h at −40° C. under nitrogen, after which the resulting mixture was diluted with water (5 mL), and extracted with EtOAc (3×10 mL). The combined organic layers were washed with brine (5 mL), dried over anhydrous Na₂SO₄, and concentrated under reduced pressure. The residue was purified by Prep-TLC (PE/EtOAc 1:1) to afford the title compound (200 mg, 39%) as an off-white solid. (ES, m/z): [M+H]⁺ 292.0; ¹H NMR (400 MHz, DMSO-d₆): δ 3.89 (s, 3H), 4.41 (d, J=8.0 Hz, 2H), 4.50 (d, J=7.6 Hz, 2H), 6.78 (s, 1H), 7.38-7.59 (m, 3H), 7.61-7.82 (m, 2H).

### EXAMPLE 108—SYNTHESIS OF METHYL 3-CHLORO-5-(4-CYCLOPROPYL-3-PHENYLISOTHIAZOLE-5-CARBOXAMIDO)PICOLINATE

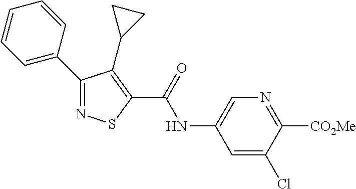 [SMILES: COC(=O)c1ncc(NC(=O)c2snc(-c3ccccc3)c2C2CC2)cc1Cl]

General procedure A (using 4-cyclopropyl-3-phenylisothiazole-5-carboxylic acid and methyl 5-amino-3-chloropicolinate) yielded the title compound (80 mg, 18%) as a white solid. (ES, m/z): [M+H]⁺ 414.

### EXAMPLE 109—SYNTHESIS OF 3-CHLORO-5-(4-CYCLOPROPYL-3-PHENYL-ISOTHIAZOLE-5-CARBOXAMIDO)PICOLINIC ACID

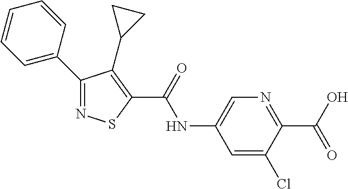 [SMILES: O=C(O)c1ncc(NC(=O)c2snc(-c3ccccc3)c2C2CC2)cc1Cl]

General procedure B (using methyl 3-chloro-5-(4-cyclopropyl-3-phenylisothiazole-5-carboxamido)picolinate) yielded the title compound (100 mg) as a white solid, which was used without further purification. (ES, m/z): [M+H]⁺ 400.

### EXAMPLE 110—SYNTHESIS OF METHYL 5-(4-CYCLOPROPYL-3-PHENYL-ISOTHIAZOLE-5-CARBOXAMIDO)PICOLINATE

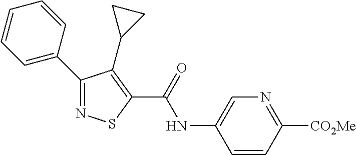 [SMILES: COC(=O)c1ccc(NC(=O)c2snc(-c3ccccc3)c2C2CC2)cn1]

General procedure A (using 4-cyclopropyl-3-phenylisothiazole-5-carboxylic acid and methyl 5-aminopicolinate) yielded the title compound (223 mg, 72%) as a white solid. (ES, m/z): [M+H]⁺ 380.1; ¹H NMR (400 MHz, CDCl₃): δ 0.50-0.60 (m, 2H), 1.08-1.15 (m, 2H), 2.16-2.25 (m, 1H), 4.03 (s, 3H), 7.47-7.56 (m. 3H), 7.70-7.75 (m, 2H), 8.24 (d. J=8.8 Hz, 1H), 8.67 (d, J=8.8 Hz, 1H), 8.88 (s, 1H), 9.28 (s, 1H).

### EXAMPLE 111—SYNTHESIS OF 5-(4-CYCLOPROPYL-3-PHENYLISOTHIAZOLE-5-CARBOXAMIDO)PICOLINIC ACID

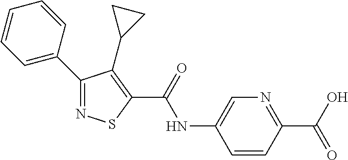 [SMILES: O=C(O)c1ccc(NC(=O)c2snc(-c3ccccc3)c2C2CC2)cn1]

General procedure B (using methyl 5-(4-cyclopropyl-3-phenylisothiazole-5-carboxamido) picolinate) yielded the title compound (250 mg) as a white solid, which was used without further purification. (ES, m/z): [M+H]⁺ 366.1; ¹H NMR (400 MHz, DMSO-d₆): δ 0.35-0.45 (m, 2H), 0.70-0.80 (m, 2H), 2.14-2.33 (m, 1H), 7.41-7.51 (m, 3H), 7.70-7.75 (m, 3H), 7.81 (s, 1H), 8.04 (d, J=8.4 Hz, 1H), 8.58 (s, 1H).

### EXAMPLE 112—SYNTHESIS OF N-(5-CHLORO-6-(2H-1,2,3-TRIAZOL-2-YL)PYRIDIN-3-YL)-4-IODO-3-PHENYLISOTHIAZOLE-5-CARBOXAMIDE (I-1)

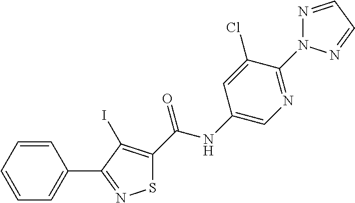 [SMILES: O=C(Nc1cnc(-n2nccn2)c(Cl)c1)c1snc(-c2ccccc2)c1I]

General Procedure A (using 4-iodo-3-phenylisothiazole-5-carboxylic acid and 5-chloro-6-(2H-1,2,3-triazol-2-yl)pyridin-3-amine) yielded the title compound (62 mg, 13%) as a light yellow solid. (ES, m/z): [M+H]⁺ 509; ¹H NMR (400 MHz, DMSO-d₆): δ 7.53-7.60 (m, 3H), 7.73-7.75 (m, 2H), 8.21 (s, 2H), 8.63 (d, J=2.4 Hz, 1H), 8.83 (d, J=2.4 Hz, 1H), 11.59 (s, 1H).

### EXAMPLE 113—SYNTHESIS OF N-(5-CHLORO-6-(2H-1,2,3-TRIAZOL-2-YL)PYRIDIN-3-YL)-3-PHENYLISOTHIAZOLE-5-CARBOXAMIDE (I-2)

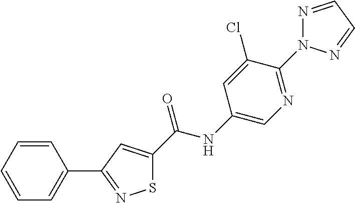 [SMILES: O=C(Nc1cnc(-n2nccn2)c(Cl)c1)c1cc(-c2ccccc2)ns1]

To a solution of N-[5-chloro-6-(1,2,3-triazol-2-yl)pyridin-3-yl]-4-iodo-3-phenyl-1,2-thiazole-5-carboxamide (150 mg, 0.3 mmol, 1 eq.) in MeOH (3 mL) was added Pd/C (10%, 60 mg) under nitrogen atmosphere in a 25 mL round-bottom flask. The mixture was hydrogenated at room temperature for 16 h under hydrogen. The resulting mixture was filtered, the solids washed with MeOH (2×10 mL), and the filtrate was concentrated under reduced pressure. The residue was purified by reverse flash chromatography with the following conditions: column, C18 silica gel, mobile phase, MeCN in Water (0.1% TFA), 10% to 50% gradient in 10 min; detector, UV 254 nm. This yielded the title compound (46 mg, 41%) as an off-white solid. (ES, m/z): [M+H]⁺ 383; ¹H NMR (400 MHz, DMSO-d₆): δ 7.50-7.60 (m, 3H), 8.04-8.06 (m, 2H), 8.20 (s, 2H), 8.67-8.70 (m, 2H), 8.89 (d, J=2.4 Hz, 1H), 11.30 (s, 1H).

### EXAMPLE 114—SYNTHESIS OF N-(5-CHLORO-6-(2H-1,2,3-TRIAZOL-2-YL)PYRIDIN-3-YL)-4-METHYL-3-PHENYLISOTHIAZOLE-5-CARBOXAMIDE (I-3)

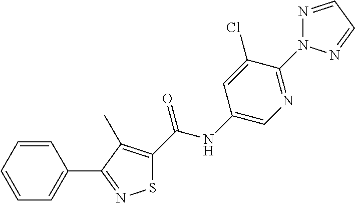 [SMILES: Cc1c(-c2ccccc2)nsc1C(=O)Nc1cnc(-n2nccn2)c(Cl)c1]

General Procedure A (using 4-methyl-3-phenylisothiazole-5-carboxylic acid and 5-chloro-6-(2H-1,2,3-triazol-2-yl)pyridin-3-amine) yielded the title compound (97 mg, 26%) as an off-white solid. (ES, m/z): [M+H]⁺ 397; ¹H NMR (400 MHz, DMSO-d₆): δ 2.50 (s, 3H), 7.51-7.58 (m, 3H), 7.66-7.71 (m, 2H), 8.19 (s, 2H), 8.64 (d, J=2.4 Hz, 1H), 8.86 (d, J=2.4 Hz, 1H), 11.28 (s, 1H)

### EXAMPLE 115—SYNTHESIS OF N-(5-CHLORO-6-(2H-1,2,3-TRIAZOL-2-YL)PYRIDIN-3-YL)-4-CYANO-3-PHENYLISOTHIAZOLE-5-CARBOXAMIDE (I-4)

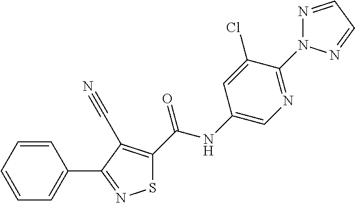 [SMILES: N#Cc1c(-c2ccccc2)nsc1C(=O)Nc1cnc(-n2nccn2)c(Cl)c1]

General Procedure A (using 4-cyano-3-phenylisothiazole-5-carboxylic acid and 5-chloro-6-(2H-1,2,3-triazol-2-yl)pyridin-3-amine) yielded the title compound (5 mg, 3%) as a white solid. (ES, m/z): [M+H]⁺ 408; ¹H NMR (400 MHz, DMSO-d₆): δ 7.62-7.64 (m, 3H), 7.95-8.00 (m, 2H), 8.21 (s, 2H), 8.66 (d, J=2.4 Hz, 1H), 8.83 (s, 1H), 11.75 (s, 1H).

### EXAMPLE 116—SYNTHESIS OF N-(5-CHLORO-6-(2H-1,2,3-TRIAZOL-2-YL) PYRIDIN-3-YL)-4-ISOPROPYL-3-PHENYLISOTHIAZOLE-5-CARBOXAMIDE (I-5)

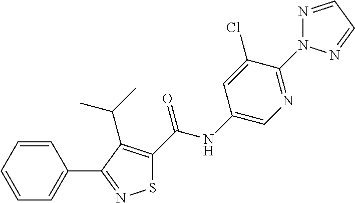 [SMILES: CC(C)c1c(-c2ccccc2)nsc1C(=O)Nc1cnc(-n2nccn2)c(Cl)c1]

General Procedure A (using 4-isopropyl-3-phenylisothiazole-5-carboxylic acid and 5-chloro-6-(2H-1,2,3-triazol-2-yl)pyridin-3-amine) yielded the title compound (154 mg, 60%) as an off-white solid. (ES, m/z): [M+H]⁺ 425; ¹H NMR (400 MHz, CDCl₃): δ 1.37 (d, J=6.8 Hz, 6H), 3.36-3.43 (m, 1H), 7.45-7.55 (m, 5H), 7.97 (s, 2H), 8.06 (s. 1H), 8.53 (d, J=2.4 Hz, 1H), 8.74 (d, J=2.4 Hz, 1H).

### EXAMPLE 117—SYNTHESIS OF N-(5-CHLORO-6-(2H-1,2,3-TRIAZOL-2-YL) PYRIDIN-3-YL)-4-CYCLOPROPYL-3-PHENYLISOTHIAZOLE-5-CARBOXAMIDE

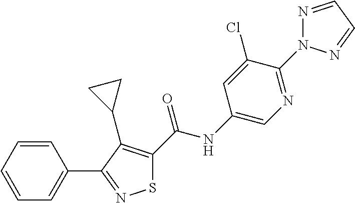 [SMILES: O=C(Nc1cnc(-n2nccn2)c(Cl)c1)c1snc(-c2ccccc2)c1C1CC1]

General Procedure A (using 4-cyclopropyl-3-phenylisothiazole-5-carboxylic acid and 5-chloro-6-(2H-1,2,3-triazol-2-yl)pyridin-3-amine) yielded the title compound (24 mg, 28%) as an off-white solid. (ES, m/z): [M+H]⁺ 423; ¹H NMR (400 MHz, CDCl₃): δ 0.60-0.69 (m, 2H), 1.18-1.21 (m, 2H), 2.18-2.22 (m, 1H), 7.51-7.54 (m, 3H), 7.75-7.76 (m, 2H), 7.98 (s, 2H), 8.57 (s, 1H), 8.82 (s, 1H), 9.01 (s, 1H).

### EXAMPLE 118—SYNTHESIS OF N-(5-CHLORO-6-(2H-1,2,3-TRIAZOL-2-YL) PYRIDIN-3-YL)-3-PHENYL-4-(TRIFLUOROMETHYL)ISOTHIAZOLE-5-CARBOXAMIDE (I-7)

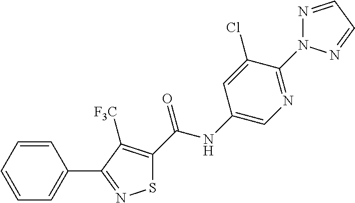 [SMILES: O=C(Nc1cnc(-n2nccn2)c(Cl)c1)c1snc(-c2ccccc2)c1C(F)(F)F]

General Procedure A (using 3-phenyl-4-(trifluoromethyl)isothiazole-5-carboxylic acid and 5-chloro-6-(2H-1,2,3-triazol-2-yl)pyridin-3-amine) yielded the title compound (41 mg, 50%). (ES, m/z): [M+H]⁺ 451.2, ¹H NMR (400 MHz, DMSO-d₆): δ 7.60-7.56 (m, 5H), 8.20 (s, 2H), 8.58 (d, J=2.3 Hz, 1H), 8.75 (d, J=2.3 Hz, 1H), 11.85 (s, 1H).

### EXAMPLE 119—SYNTHESIS OF N-(5-CYANO-6-(2H-1,2,3-TRIAZOL-2-YL)PYRIDIN-3-YL)-3-PHENYL-4-(TRIFLUOROMETHYL)ISOTHIAZOLE-5-CARBOXAMIDE (I-8)

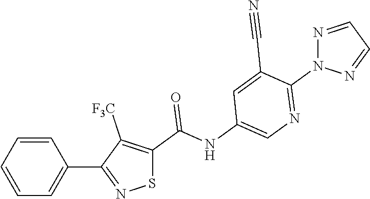 [SMILES: N#Cc1cc(NC(=O)c2snc(-c3ccccc3)c2C(F)(F)F)cnc1-n1nccn1]

General Procedure A (using 3-phenyl-4-(trifluoromethyl)isothiazole-5-carboxylic acid and 5-cyano-6-(2H-1,2,3-triazol-2-yl)pyridin-3-amine) yielded the title compound (51 mg, 50%). (ES, m/z): [M+H]⁺ 442.2, ¹H NMR (400 MHz, DMSO-d₆): δ 7.63-7.54 (m, 5H), 8.34-8.34 (m, 2H), 8.79-8.78 (m, 1H), 9.03-9.01 (m, 1H), 11.92 (s, 1H).

### EXAMPLE 120—SYNTHESIS OF N-(5-CYANO-6-(2H-1,2,3-TRIAZOL-2-YL) PYRIDIN-3-YL)-4-CYCLOPROPYL-3-PHENYLISOTHIAZOLE-5-CARBOXAMIDE

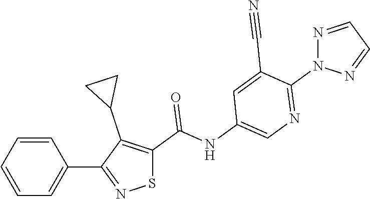 [SMILES: N#Cc1cc(NC(=O)c2snc(-c3ccccc3)c2C2CC2)cnc1-n1nccn1]

General Procedure A (using 4-cyclopropyl-3-phenylisothiazole-5-carboxylic acid and 5-cyano-6-(2H-1,2,3-triazol-2-yl)pyridin-3-amine) yielded the title compound (15 mg, 11%) as a white solid. (ES, m/z): [M+H]⁺ 414, ¹H NMR (400 MHz, DMSO-d₆): δ 0.33-0.37 (m, 2H), 0.87-0.91 (m, 2H), 2.23-2.34 (m, 1H), 7.52-7.57 (m, 3H), 7.85-7.87 (m, 2H), 8.33 (s, 2H), 8.85 (d, J=2.4 Hz, 1H), 9.09 (d, J=2.4 Hz, 1H), 11.37 (s, 1H).

### EXAMPLE 121—SYNTHESIS OF N-(5-CHLORO-6-(2H-1,2,3-TRIAZOL-2-YL) PYRIDIN-3-YL)-4-VINYL-3-PHENYLISOTHIAZOLE-5-CARBOXAMIDE (1-10)

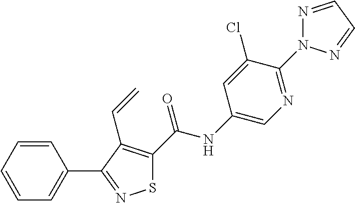 [SMILES: C=Cc1c(-c2ccccc2)nsc1C(=O)Nc1cnc(-n2nccn2)c(Cl)c1]

General Procedure A (using 3-phenyl-4-vinylisothiazole-5-carboxylic acid and 5-chloro-6-(2H-1,2,3-triazol-2-yl)pyridin-3-amine) yielded the title compound (55 mg, 24%) as a white solid. (ES, m/z): [M+H]⁺ 409, ¹H NMR (400 MHz, DMSO-d₆): δ 5.40-5.60 (m, 2H), 6.70-6.90 (m, 1H), 7.45-7.60 (m, 3H), 7.60-7.75 (m, 2H), 8.18 (s, 2H), 8.60 (s, 1H), 8.77 (s, 1H), 11.46 (s, 1H).

### EXAMPLE 122—SYNTHESIS OF N-(5-CHLORO-6-(2H-1,2,3-TRIAZOL-2-YL) PYRIDIN-3-YL)-4-ETHYL-3-PHENYLISOTHIAZOLE-5-CARBOXAMIDE (I-11)

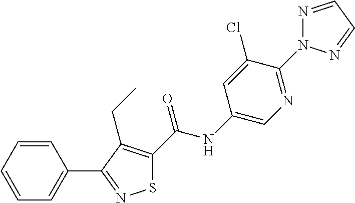 [SMILES: CCc1c(-c2ccccc2)nsc1C(=O)Nc1cnc(-n2nccn2)c(Cl)c1]

General Procedure A (using 4-ethyl-3-phenylisothiazole-5-carboxylic acid and 5-chloro-6-(2H-1,2,3-triazol-2-yl)pyridin-3-amine) yielded the title compound (13 mg, 7%) as a white solid. (ES, m/z): [M+H]⁺ 411, ¹H NMR (400 MHz, DMSO-d₆): δ 1.06 (t, J=7.6 Hz, 3H), 2.93 (q, J=7.6 Hz, 2H), 7.50-7.60 (m, 3H), 7.60-7.65 (m, 2H), 8.19 (s, 2H), 8.64 (d, J=2.4 Hz, 1H), 8.85 (d, J=2.4 Hz, 1H), 11.35 (s, 1H).

### EXAMPLE 123—SYNTHESIS OF 4-CYCLOPROPYL-N-(5-METHYL-6-(2H-1,2,3-TRIAZOL-2-YL)PYRIDIN-3-YL)-3-PHENYLISOTHIAZOLE-5-CARBOXAMIDE (I-12

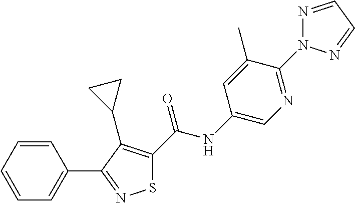 [SMILES: Cc1cc(NC(=O)c2snc(-c3ccccc3)c2C2CC2)cnc1-n1nccn1]

General Procedure A (using 4-cyclopropyl-3-phenylisothiazole-5-carboxylic acid and 5-methyl-6-(2H-1,2,3-triazol-2-yl)pyridin-3-amine) yielded the title compound (54 mg, 33%) as a white solid. (ES, m/z): [M+H]⁺ 403, ¹H NMR (400 MHz, DMSO-d₆): δ 0.38-0.43 (m, 2H), 0.85-0.91 (m, 2H), 2.11-2.29 (m, 4H), 7.48-7.78 (m, 3H), 7.79-7.98 (m, 2H), 8.15 (s, 2H), 8.34 (d, J=2 Hz, 1H), 8.75 (d, J=2.0 Hz, 1H), 11.11 (s, 1H).

### EXAMPLE 124—SYNTHESIS OF 4-CYCLOPROPYL-N-(5-(DIFLUOROMETHYL)-6-(2H-1,2,3-TRIAZOL-2-YL)PYRIDIN-3-YL)-3-PHENYLISOTHIAZOLE-5-CARBOXAMIDE (I-13)

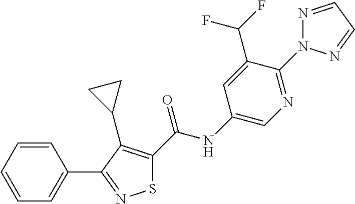 [SMILES: O=C(Nc1cnc(-n2nccn2)c(C(F)F)c1)c1snc(-c2ccccc2)c1C1CC1]

General Procedure A (using 4-cyclopropyl-3-phenylisothiazole-5-carboxylic acid and 5-(difluoromethyl)-6-(2H-1,2,3-triazol-2-yl)pyridin-3-amine) yielded the title compound (57 mg, 32%) as a white solid. (ES, m/z): [M+H]⁺ 439, ¹H NMR (400 MHz, DMSO-d₆): δ 0.35-0.48 (m, 2H), 0.90-0.98 (m, 2H), 2.11-2.29 (m, 1H), 7.39 (t, J=140.0 Hz, 1H), 7.45-7.67 (m, 3H), 7.85 (dd, J=2.0, 8.0 Hz, 2H), 8.24 (s, 2H), 8.75 (d, J=2.0 Hz, 1H), 9.33 (d, J=2.0 Hz, 1H), 11.33 (s, 1H).

### EXAMPLE 125—SYNTHESIS OF 4-CYCLOPROPYL-N-(5-(TRIFLUOROMETHYL)-6-(2H-1,2,3-TRIAZOL-2-YL)PYRIDIN-3-YL)-3-PHENYLISOTHIAZOLE-5-CARBOXAMIDE (I-14)

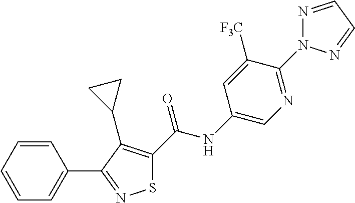 [SMILES: O=C(Nc1cnc(-n2nccn2)c(C(F)(F)F)c1)c1snc(-c2ccccc2)c1C1CC1]

General Procedure A (using 4-cyclopropyl-3-phenylisothiazole-5-carboxylic acid and 6-(2H-1,2,3-triazol-2-yl)-5-(trifluoromethyl)pyridin-3-amine) yielded the title compound (49 mg, 26%) as a white solid. (ES, m/z): [M+H]⁺ 457, ¹H NMR (400 MHz, DMSO-d₆): δ 0.30-0.49 (m, 2H), 0.73-0.99 (m, 2H), 2.11-2.30 (m, 1H), 7.40-7.70 (m, 3H), 7.72-8.01 (m, 2H), 8.22 (s, 2H), 8.89 (s, 1H), 9.15 (s, 1H), 11.50 (s, 1H).

### EXAMPLE 126—SYNTHESIS OF 3-PHENYL-4-(TRIFLUOROMETHYL)-N-(2-(TRIFLUOROMETHYL)PYRIDIN-4-YL)ISOTHIAZOLE-5-CARBOXAMIDE (I-15)

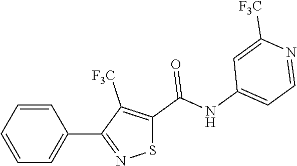 [SMILES: O=C(Nc1ccnc(C(F)(F)F)c1)c1snc(-c2ccccc2)c1C(F)(F)F]

General Procedure A (using 3-phenyl-4-(trifluoromethyl)isothiazole-5-carboxylic acid and 2-(trifluoromethyl)pyridin-4-amine) yielded the title compound (46 mg, 60%). (ES, m/z): [M+H]⁺ 418.2, ¹H NMR (400 MHz, DMSO-d₆): δ 7.61-7.52 (m, 5H), 7.83 (dd, J=1.8, 5.5 Hz, 1H), 8.15 (d, J=1.8 Hz, 1H), 8.73 (d, J=5.5 Hz, 1H), 11.83 (s, 1H).

### EXAMPLE 127—SYNTHESIS OF 3-PHENYL-4-CYCLOPROPYL-N-(2-(TRIFLUOROMETHYL)PYRIDIN-4-YL)ISOTHIAZOLE-5-CARBOXAMIDE (I-26)

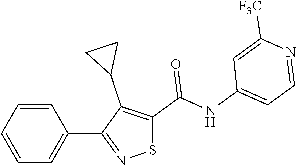 [SMILES: O=C(Nc1ccnc(C(F)(F)F)c1)c1snc(-c2ccccc2)c1C1CC1]

General Procedure A (using 4-cyclopropyl-3-phenylisothiazole-5-carboxylic acid and 2-(trifluoromethyl)pyridin-4-amine) yielded the title compound (57 mg, 24%) as an off-white solid. (ES, m/z): [M+H]⁺ 390, ¹H NMR (400 MHz, DMSO-d₆): δ 0.35-0.47 (m, 2H), 0.85-0.97 (m, 2H), 2.13-2.28 (m, 1H), 7.39-7.68 (m, 3H), 7.84 (dd, J=2.0, 8.0, 2H), 7.94 (dd, J=2.0, 5.6 Hz, 1H), 8.22 (d, J=2.0 Hz, 1H), 8.73 (d, J=5.6 Hz, 1H), 11.37 (s, 1H).

### EXAMPLE 128—SYNTHESIS OF N-(4-(3-((TERT-BUTYLDIMETHYLSILYL) OXY)AZETIDINE-1-CARBONYL)-3-CHLOROPHENYL)-3-PHENYL-4-(TRIFLUOROMETHYL)ISOTHIAZOLE-5-CARBOXAMIDE

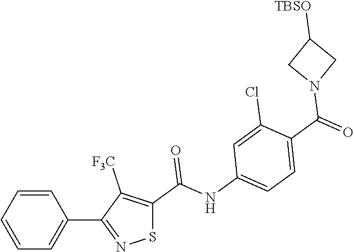 [SMILES: CC(C)(C)[Si](C)(C)OC1CN(C(=O)c2ccc(NC(=O)c3snc(-c4ccccc4)c3C(F)(F)F)cc2Cl)C1]

General Procedure A (using 3-phenyl-4-(trifluoromethyl)isothiazole-5-carboxylic acid and 4-amino-2-chlorophenyl)(3-((tert-butyldimethylsilyl)oxy)azetidin-1-yl)methanone) yielded the title compound (380 mg, 43%) as a yellow solid. (ES, m/z): [M+H]⁺ 596.

### EXAMPLE 129—SYNTHESIS OF N-(3-CHLORO-4-(3-HYDROXYAZETIDINE-1-CARBONYL)PHENYL)-3-PHENYL-4-(TRIFLUOROMETHYL)ISOTHIAZOLE-5-CARBOXAMIDE (I-16)

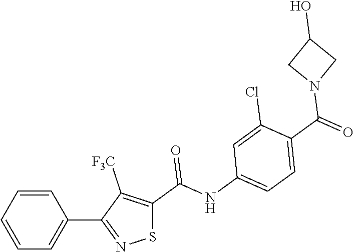 [SMILES: O=C(Nc1ccc(C(=O)N2CC(O)C2)c(Cl)c1)c1snc(-c2ccccc2)c1C(F)(F)F]

A mixture of N-(4-(3-((tert-butyldimethylsilyl)oxy)azetidine-1-carbonyl)-3-chlorophenyl)-3-phenyl-4-(trifluoromethyl)isothiazole-5-carboxamide (280 mg, 0.5 mmol, 1 eq.) and CsF (143 mg, 0.9 mmol, 2 eq.) in MeOH (5 mL) was stirred for 2 h at room temperature under nitrogen. After filtration, the filtrate was concentrated under reduced pressure, and the residue was purified by reverse flash chromatography with the following conditions: column, C18 silica gel; mobile phase, MeCN in Water, 40% to 60% gradient in 10 min; detector, UV 254 nm. This yielded the title compound (106 mg, 47%) as an off-white solid. (ES, m/z): [M+H]⁺ 482; ¹H NMR (400 MHz, DMSO-d₆): δ 3.68-3.78 (m, 2H), 4.04 (t, J=8.0 Hz, 1H), 4.22 (t, J=8.0 Hz, 1H), 4.49-4.53 (m, 1H), 5.81 (d, J=6.0 Hz, 1H), 7.45 (d, J=8.4 Hz, 1H), 7.53-7.60 (m, 6H), 7.89 (d, J=2.0 Hz, 1H), 11.39 (s, 1H).

### EXAMPLE 130—SYNTHESIS OF N-(4-(3-((TERT-BUTYLDIMETHYLSILYL) OXY)AZETIDINE-1-CARBONYL)-3-CHLOROPHENYL)-4-CYCLOPROPYL-3-PHENYLISOTHIAZOLE-5-CARBOXAMIDE

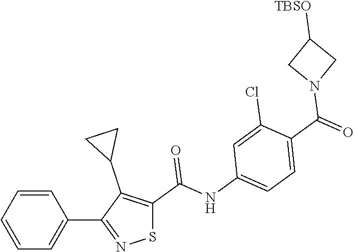 [SMILES: CC(C)(C)[Si](C)(C)OC1CN(C(=O)c2ccc(NC(=O)c3snc(-c4ccccc4)c3C3CC3)cc2Cl)C1]

General Procedure A (using 4-cyclopropyl-3-phenylisothiazole-5-carboxylic acid and (4-amino-2-chlorophenyl)(3-((tert-butyldimethylsilyl)oxy)azetidin-1-yl)methanone) yielded the title compound (80 mg, 35%) as an off-white solid. (ES, m/z): [M+H]⁺ 568. ¹H NMR (400 MHz, DMSO-d₆): δ 0.01 (s, 6H), 0.25-0.41 (m, 2H), 0.73-0.92 (m, 2H), 0.83 (s, 9H), 2.12-2.27 (m, 1H), 3.70-3.85 (m, 2H), 4.05-4.15 (m, 1H), 4.15-4.30 (m, 1H), 4.42-4.60 (m, 1H), 7.45-7.59 (m, 4H), 7.62-7.72 (m, 1H), 7.81-7.89 (m, 2H), 7.94 (s, 1H), 10.92 (s, 1H).

### EXAMPLE 131—SYNTHESIS OF N-(3-CHLORO-4-(3-HYDROXYAZETIDINE-1-CARBONYL)PHENYL)-3-PHENYL-4—(CYCLOPROPYL)ISOTHIAZOLE-5-CARBOXAMIDE (I-17)

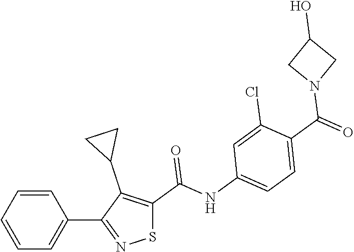 [SMILES: O=C(Nc1ccc(C(=O)N2CC(O)C2)c(Cl)c1)c1snc(-c2ccccc2)c1C1CC1]

A mixture of N-(4-(3-((tert-butyldimethylsilyl)oxy)azetidine-1-carbonyl)-3-chlorophenyl)-4-cyclopropyl-3-phenylisothiazole-5-carboxamide (82 mg, 0.14 mmol, 1.0 eq.) and caesium fluoride (66 mg, 0.43 mmol, 3.0 eq.) in MeOH (5 mL) was stirred for 1 h at 50° C. under nitrogen. The resulting mixture was concentrated under reduced pressure, and the residue was purified by Prep-HPLC with the following conditions: Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 32% B to 52% B in 8 min, 52% B; Wave Length: 220/254 nm; RT1(min): 7.68, to afford the title compound (39 mg, 59%) as an off-white solid. (ES, m/z): [M+H]⁺ 454, ¹H NMR (400 MHz, DMSO-d₆): δ 0.30-0.41 (m, 2H), 0.80-0.92 (m, 2H), 2.12-2.27 (m, 1H), 3.63-3.81 (m, 2H), 4.05-4.15 (m, 1H), 4.15-4.30 (m, 1H), 4.42-4.60 (m, 1H), 5.83 (d, J=6.0 Hz, 1H), 7.45 (d, J=8.4 Hz, 1H), 7.48-7.59 (m, 3H), 7.62-7.72 (m, 1H), 7.81-7.89 (m, 2H), 7.94 (d, J=1.2 Hz, 1H), 10.92 (s, 1H).

### EXAMPLE 132—SYNTHESIS OF N-(5-CHLORO-6-(2H-1,2,3-TRIAZOL-2-YL) PYRIDIN-3-YL)-N-METHYL-3-PHENYL-4-(TRIFLUOROMETHYL)ISOTHIAZOLE-5-CARBOXAMIDE (1-18)

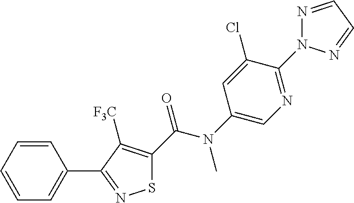 [SMILES: CN(C(=O)c1snc(-c2ccccc2)c1C(F)(F)F)c1cnc(-n2nccn2)c(Cl)c1]

NaH (60%, 50 mg, 1.3 mmol, 1.5 eq.) was added to a solution of N-[5-chloro-6-(1,2,3-triazol-2-yl)pyridin-3-yl]-3-phenyl-4-(trifluoromethyl)-1,2-thiazole-5-carboxamide (400 mg, 0.9 mmol, 1.0 eq.) in THF (2 mL) at 0° C. under nitrogen atmosphere and stirred for 20 min. To the above mixture was added MeI (189 mg, 1.3 mmol, 1.5 eq.) dropwise at 0° C. The resulting mixture was stirred for additional 8 h at 40° C. The resulting mixture was quenched with water (20 mL), and was extracted with EtOAc (2×20 mL). The combined organic layers were washed with brine (40 mL), dried over anhydrous Na₂SO₄. After filtration, the filtrate was concentrated under reduced pressure. The residue was purified by reverse flash chromatography with the following conditions: column, C18 silica gel; mobile phase, MeCN in Water (0.1% TFA), 10% to 50% gradient in 10 min; detector, UV 254 nm. The crude product was purified again by Prep-HPLC with the following conditions (Column: XBridge Shield RP18 OBD Column, 30*150 mm, 5 μm; Mobile Phase A: Water (10 mmol/L NH4HCO3+0.1% NH3·H2O), Mobile Phase B: ACN; Flow rate: 60 mL/min; Gradient: 39% B to 57% B in 11 min, 57% B; Wave Length: 220/254 nm; RT1(min): 10.58; Number Of Runs: 0) to afford the title compound (73 mg, 17%) as a white solid. (ES, m/z): [M+H]⁺ 465; ¹H NMR (400 MHz, 80° C., DMSO-d₆): δ 3.48 (s, 3H), 7.49-7.58 (m, 5H), 8.14 (s, 2H), 8.43 (d, J=2.4 Hz, 1H), 8.66 (s, 1H).

### EXAMPLE 133—SYNTHESIS OF N-(5-CHLORO-6-(2H-1,2,3-TRIAZOL-2-YL) PYRIDIN-3-YL)-3-(3-CHLOROPYRIDIN-2-YL)-4-(TRIFLUOROMETHYL) ISOTHIAZOLE-5-CARBOXAMIDE (1-19)

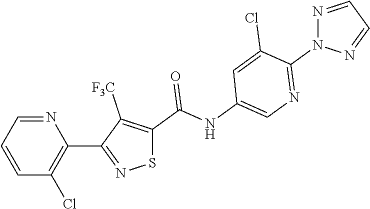 [SMILES: O=C(Nc1cnc(-n2nccn2)c(Cl)c1)c1snc(-c2ncccc2Cl)c1C(F)(F)F]

General Procedure A (using 3-(3-chloropyridin-2-yl)-4-(trifluoromethyl)isothiazole-5-carboxylic acid and 5-chloro-6-(2H-1,2,3-triazol-2-yl)pyridin-3-amine) yielded the title compound (30 mg, 40%) as an off-white solid. (ES, m/z): [M+H]⁺ 486, ¹H NMR (400 MHz, DMSO-d₆): δ 7.66 (1H, dd, J=4.8, 8.1 Hz), 8.19 (2H, s), 8.21 (1H, dd, J=1.5, 8.3 Hz), 8.59 (1H, d, J=2.3 Hz), 8.69 (1H, dd, J=1.4, 4.6 Hz), 8.76 (1H, d, J=2.3 Hz), 11.91 (1H, br. s).

### EXAMPLE 134—SYNTHESIS OF 3-(3-CHLOROPYRIDIN-2-YL)-N-(5-CYANO-6-(2H-1,2,3-TRIAZOL-2-YL)PYRIDIN-3-YL)-4-(TRIFLUOROMETHYL)ISOTHIAZOLE-5-CARBOXAMIDE (I-20)

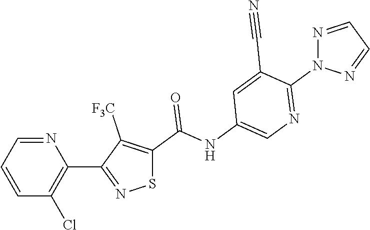 [SMILES: N#Cc1cc(NC(=O)c2snc(-c3ncccc3Cl)c2C(F)(F)F)cnc1-n1nccn1]

General Procedure A (using 3-(3-chloropyridin-2-yl)-4-(trifluoromethyl)isothiazole-5-carboxylic acid and 5-cyano-6-(2H-1,2,3-triazol-2-yl)pyridin-3-amine) yielded the title compound (26 mg, 32%) as an off-white solid. (ES, m/z): [M+H]⁺ 477; ¹H NMR (400 MHz, DMSO-d₆): δ 7.66 (dd, J=4.8, 8.3 Hz, 1H), 8.20 (dd, J=1.4, 8.2 Hz, 1H), 8.31 (s, 2H), 8.68 (dd, J=1.3, 4.8 Hz, 1H), 8.78 (d, J=2.5 Hz, 1H), 8.99 (br. s, 1H), 11.94 (br. s, 1H).

### EXAMPLE 135—SYNTHESIS OF METHYL 2-CHLORO-4-(4-CYCLOPROPYL-3-PHENYLISOTHIAZOLE-5-CARBOXAMIDO)BENZOATE

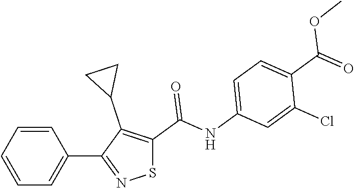 [SMILES: COC(=O)c1ccc(NC(=O)c2snc(-c3ccccc3)c2C2CC2)cc1Cl]

A mixture of 4-cyclopropyl-3-phenyl-1,2-thiazole-5-carboxylic acid (400 mg, 1.6 mmol, 1.0 eq) and SOCl₂ (5 mL) in THF (5 mL) was stirred for 1 h at room temperature under nitrogen. The resulting mixture was concentrated under vacuum, and the residue was dissolved in DCM (20 mL), and then methyl 4-amino-2-chlorobenzoate (280 mg, 1.6 mmol, 1.0 eq) and TEA (495 mg, 4.9 mmol, 3.0 eq) were added. The resulting mixture was stirred for 2 h at room temperature under nitrogen. The resulting mixture was diluted with water (50 mL) and then extracted with DCM (3×20 mL), the combined organic layers were washed with brine (50 mL), dried over anhydrous Na₂SO₄ and concentrated under reduced pressure. The residue was purified by reversed-phase flash chromatography with the following conditions: column, C18 silica gel; mobile phase, MeCN in Water, 0% to 100% gradient in 30 min; detector, UV 254 nm to afford the title compound (320 mg, 49%) as an off-white solid. (ES, m/z): [M+H]⁺ 413; ¹H NMR (400 MHz, DMSO-d₆): δ 0.28-0.37 (m, 2H), 0.79-0.90 (m, 2H), 2.22 (dd, J=8.4, 5.4 Hz, 1H), 3.86 (s, 3H), 7.47-7.59 (m, 3H), 7.77 (dd, J=8.6, 2.1 Hz, 1H), 7.88-7.81 (m, 2H), 7.94 (d, J=8.6 Hz, 1H), 8.02 (d, J=2.0 Hz, 1H), 11.08 (s, 1H).

### EXAMPLE 136—SYNTHESIS OF 2-CHLORO-4-(4-CYCLOPROPYL-3-PHENYL-ISOTHIAZOLE-5-CARBOXAMIDO)BENZOIC ACID

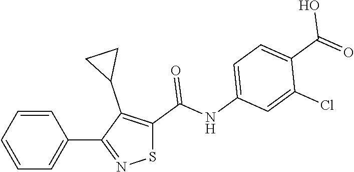 [SMILES: O=C(O)c1ccc(NC(=O)c2snc(-c3ccccc3)c2C2CC2)cc1Cl]

A mixture of methyl 2-chloro-4-(4-cyclopropyl-3-phenyl-1,2-thiazole-5-amido)benzoate (330 mg, 0.8 mmol, 1.0 eq) and LiOH (57 mg, 2.4 mmol, 3.0 eq) in MeOH (2 mL) and water (2 mL) was stirred for 2 h at room temperature under nitrogen. The mixture was then acidified to pH-4 with 2 M HCl, diluted with water (10 mL), and was extracted with DCM (3×5 mL). The combined organic layers were washed with brine (30 mL), dried over anhydrous Na₂SO₄, and concentrated under reduced pressure to afford the title compound (270 mg, 85%) as a pink solid. (ES, m/z): [M+H]⁺ 399; ¹H NMR (400 MHz, DMSO-d₆): δ 0.23-0.41 (m, 2H), 0.77-0.87 (m, 2H), 2.21 (dd, J=5.5, 8.4 Hz, 1H), 7.47-7.58 (m, 3H), 7.73 (dd, J=2.1, 8.6, Hz, 1H), 7.81-7.87 (m, 2H), 7.91 (d, J=8.5 Hz, 1H), 7.99 (d, J=2.0 Hz, 1H), 11.01 (s, 1H), 13.23 (s, 1H).

### EXAMPLE 137—SYNTHESIS OF N-(3-CHLORO-4-(DIMETHYLCARBAMOYL) PHENYL)-4-CYCLOPROPYL-3-PHENYLISOTHIAZOLE-5-CARBOXAMIDE (I-21)

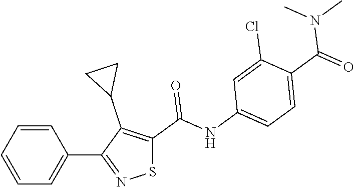 [SMILES: CN(C)C(=O)c1ccc(NC(=O)c2snc(-c3ccccc3)c2C2CC2)cc1Cl]

General Procedure C (using 2-chloro-4-(4-cyclopropyl-3-phenylisothiazole-5-carboxamido)benzoic acid and dimethylamine) yielded the title compound (14 mg, 19%) as a white solid. (ES, m/z): [M+H]⁺ 426, ¹H NMR (400 MHz, DMSO-d₆): δ 0.30-0.40 (m, 2H), 0.85-0.92 (m, 2H), 2.20-2.35 (m, 1H), 2.81 (s, 3H), 3.01 (s, 3H), 7.39 (d, J=8.0 Hz, 1H), 7.45-7.55 (m, 3H), 7.70 (d, J=2.0 Hz, 1H), 7.84 (dd, J=2.0, 8.0 Hz, 1H), 7.96 (d, J=2.0 Hz, 1H), 11.90 (s, 1H).

### EXAMPLE 138—SYNTHESIS OF N-(3-CHLORO-4-(MORPHOLINE-4-CARBONYL) PHENYL)-4-CYCLOPROPYL-3-PHENYLISOTHIAZOLE-5-CARBOXAMIDE (I-22)

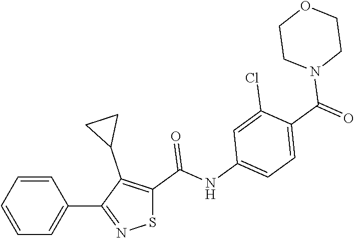 [SMILES: O=C(Nc1ccc(C(=O)N2CCOCC2)c(Cl)c1)c1snc(-c2ccccc2)c1C1CC1]

General Procedure C (using 2-chloro-4-(4-cyclopropyl-3-phenylisothiazole-5-carboxamido)benzoic acid and morpholine) yielded the title compound (18 mg, 25%) as a white solid. (ES, m/z): [M+H]⁺ 468.0, ¹H NMR (400 MHz, DMSO-d₆): δ 0.30-0.40 (m, 2H), 0.85-0.92 (m, 2H), 2.20-2.35 (m, 1H), 3.15-3.20 (m, 2H), 3.55-3.60 (m, 2H), 3.60-3.750 (m, 4H), 7.42 (d, J=8.0 Hz, 1H), 7.45-7.55 (m, 3H), 7.70 (d, J=2.0 Hz, 1H), 7.84 (dd, J=2.0, 8.0 Hz, 1H), 7.96 (d, J=2.0 Hz, 1H), 11.93 (s, 1H).

### EXAMPLE 139—SYNTHESIS OF N-(3-CHLORO-4-(METHYLCARBAMOYL) PHENYL)-4-CYCLOPROPYL-3-PHENYLISOTHIAZOLE-5-CARBOXAMIDE (I-23)

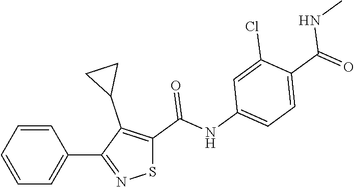 [SMILES: CNC(=O)c1ccc(NC(=O)c2snc(-c3ccccc3)c2C2CC2)cc1Cl]

General Procedure C (using 2-chloro-4-(4-cyclopropyl-3-phenylisothiazole-5-carboxamido)benzoic acid and methanamine hydrochloride) yielded the title compound (15 mg, 20%) as a white solid. (ES, m/z): [M+H]⁺ 412, ¹H NMR (400 MHz, DMSO-d₆) δ 0.30-0.40 (m, 2H), 0.79-0.86 (m, 2H), 2.16-2.24 (m, 1H), 2.75 (d, J=4.5 Hz, 3H), 7.45-7.56 (m, 4H), 7.66 (dd, J=8.3, 1.9 Hz, 1H), 7.81-7.86 (m, 2H), 7.92 (d, J=1.9 Hz, 1H), 8.31 (d, J=5.0 Hz, 1H), 10.89 (s, 1H).

### EXAMPLE 140—SYNTHESIS OF N-(3-CHLORO-4-((2-HYDROXYETHYL) (METHYL)CARBAMOYL)PHENYL)-4-CYCLOPROPYL-3-PHENYLISOTHIAZOLE-5-CARBOXAMIDE (I-24)

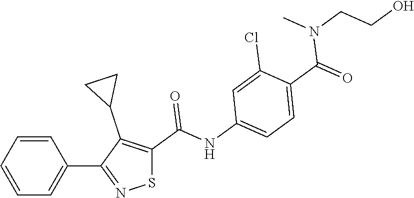 [SMILES: CN(CCO)C(=O)c1ccc(NC(=O)c2snc(-c3ccccc3)c2C2CC2)cc1Cl]

General Procedure C (using 2-chloro-4-(4-cyclopropyl-3-phenylisothiazole-5-carboxamido)benzoic acid and 2-(methylamino)ethanol hydrochloride) yielded the title compound (55 mg, 47%) as a white solid. (ES, m/z): [M+H]⁺ 456, ¹H NMR (400 MHz, DMSO-d₆) S 0.30-0.41 (m, 2H), 0.80-0.89 (m, 2H), 2.17-2.23 (m, 1H), 2.93 (d, J=68.7 Hz, 3H), 3.16 (s, 1H), 3.53 (d, J=5.9 Hz, 1H), 3.63 (q, J=6.0 Hz, 1H), 4.72-4.83 (m, 1H), 7.39 (dd, J=8.4, 1.9 Hz, 1H), 7.46-7.58 (m, 3H), 7.62-7.73 in, 1H), 7.80-7.88 (m, 2H), 7.95 (dd, J=4.8, 2.0 Hz, 1H), 10.89 (s, 1H).

### EXAMPLE 141—SYNTHESIS OF N-(3-CHLORO-4-((2-METHOXYETHYL) (METHYL) CARBAMOYL)PHENYL)-4-CYCLOPROPYL-3-PHENYL-ISOTHIAZOLE-5-CARBOXAMIDE (I-25)

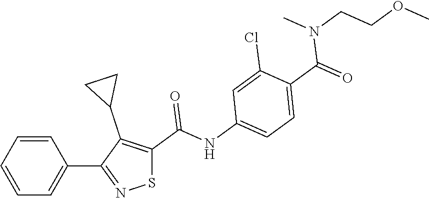 [SMILES: COCCN(C)C(=O)c1ccc(NC(=O)c2snc(-c3ccccc3)c2C2CC2)cc1Cl]

General Procedure C (using 2-chloro-4-(4-cyclopropyl-3-phenylisothiazole-5-carboxamido)benzoic acid and 2-methoxy-N-methylethanamine hydrochloride) yielded the title compound (31 mg, 26%) as a white solid. (ES, m/z): [M+H]⁺ 470, ¹H NMR (400 MHz, CDCl₃) δ 0.56 (q, J=5.5, 5.4, 5.4 Hz, 2H), 1.11 (q, J=5.9, 5.9, 5.9 Hz, 2H), 2.10-2.16 (m, 1H), 3.06 (d, J=77.2 Hz, 3H), 3.24-3.56 (m, 5H), 3.65-3.70 (m, 1H), 7.31 (dd, J=8.3, 5.8 Hz, 1H), 7.45-7.57 (m, 4H), 7.74 (dd, J=7.2, 2.5 Hz, 2H), 7.79-7.87 (m, 1H), 8.87 (s, 1H).

### EXAMPLE 142—SYNTHESIS OF 4-CYCLOPROPYL-N-(2-METHYL-1-OXOISOINDOLIN-5-YL)-3-PHENYLISOTHIAZOLE-5-CARBOXAMIDE (I-62)

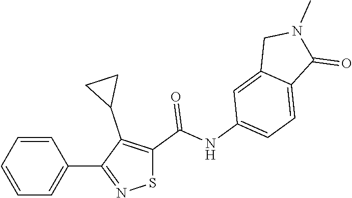 [SMILES: CN1Cc2cc(NC(=O)c3snc(-c4ccccc4)c3C3CC3)ccc2C1=O]

General procedure A (using 4-cyclopropyl-3-phenyl-1,2-thiazole-5-carboxylic acid and 5-amino-2-methyl-3H-isoindol-1-one) yielded the title compound (55 mg, 11%) as a white solid. (ES, m/z): [M+H]⁺ 390.1; ¹H NMR (DMSO-d₆, 400 MHz, ppm): δ 0.30-0.41 (m, 2H), 0.80-0.88 (m, 2H), 2.18-2.25 (m, 1H) 4.48 (s, 2H), 3.07 (s, 3H) 7.46-7.57 (m, 3H), 7.65-7.73 (m, 2H), 7.80-7.88 (m, 2H), 8.07 (s, 1H), 10.92 (s, 1H).

### EXAMPLE 143—SYNTHESIS OF N-(5-CYANO-6-(2H-1,2,3-TRIAZOL-2-YL) PYRIDIN-3-YL)-4-CYCLOPROPYL-3-(PYRIDIN-2-YL)ISOTHIAZOLE-5-CARBOXAMIDE (I-27)

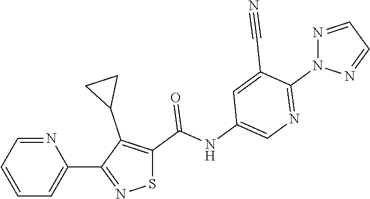 [SMILES: N#Cc1cc(NC(=O)c2snc(-c3ccccn3)c2C2CC2)cnc1-n1nccn1]

General Procedure A (using 4-cyclopropyl-3-(pyridin-2-yl)isothiazole-5-carboxylic acid and 5-amino-2-(2H-1,2,3-triazol-2-yl)nicotinonitrile) yielded the title compound (47 mg, 28%) as a white solid. (ES, m/z): [M+H]⁺ 415.0, ¹H NMR (400 MHz, DMSO-d₆): δ 0.30-0.40 (m, 2H), 0.85-0.92 (m, 2H), 2.20-2.35 (m, 1H), 7.50-7.60 (m, 1H), 7.90 (d, J=7.6 Hz, 1H), 7.95-8.05 (m, 1H), 8.32 (s, 2H), 8.70-8.80 (m, 1H), 8.84 (d, J=2.8 Hz, 1H), 9.09 (d, J=2.8 Hz, 1H), 11.42 (s, 1H).

### EXAMPLE 144—SYNTHESIS OF 3-(3-CHLOROPYRIDIN-2-YL)-N-(5-CYANO-6-(2H-1,2,3-TRIAZOL-2-YL)PYRIDIN-3-YL)-4-CYCLOPROPYLISOTHIAZOLE-5-CARBOXAMIDE (I-28)

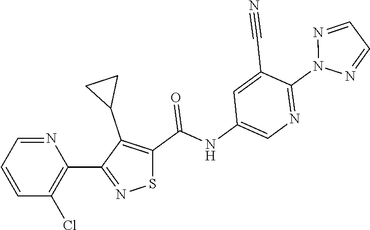 [SMILES: N#Cc1cc(NC(=O)c2snc(-c3ncccc3Cl)c2C2CC2)cnc1-n1nccn1]

General Procedure A (using 3-(3-chloropyridin-2-yl)-4-cyclopropylisothiazole-5-carboxylic acid and 5-amino-2-(2H-1,2,3-triazol-2-yl)nicotinonitrile) yielded the title compound (51 mg, 40%) as a white solid. (ES, m/z): [M+H]⁺ 449.1; ¹H NMR (400 MHz, DMSO-d₆): δ 0.24-0.43 (m, 2H), 0.59-0.78 (m, 2H), 2.05-2.15 (m, 1H), 7.63 (dd, J=8.0, 4.8 Hz, 1H), 8.18 (dd, J 8.0, 1.2 Hz, 1H), 8.33 (s, 2H), 8.70 (dd, J=4.8, 1.2 Hz, 1H), 8.86 (d, J=2.4 Hz, 1H), 9.11 (d, J=2.4 Hz, 1H), 11.36 (s, 1H).

### EXAMPLE 145—SYNTHESIS OF N-(5-CYANO-6-(DIMETHYLAMINO)PYRIDIN-3-YL)-4-CYCLOPROPYL-3-PHENYLISOTHIAZOLE-5-CARBOXAMIDE (I-29)

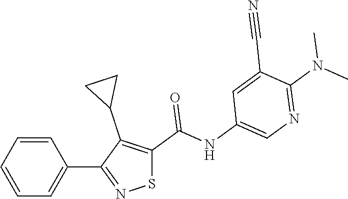 [SMILES: CN(C)c1ncc(NC(=O)c2snc(-c3ccccc3)c2C2CC2)cc1C#N]

General Procedure A (using 4-cyclopropyl-3-phenylisothiazole-5-carboxylic acid and 5-amino-2-(dimethylamino)nicotinonitrile) yielded the title compound (120 mg, 71%) as a white solid. (ES, m/z): [M+H]⁺ 390.0, H NMR (400 MHz, DMSO-d₆): δ 0.30-0.40 (m, 2H), 0.85-0.92 (m, 2H), 2.20-2.35 (m, 1H), 3.21 (s, 6H), 7.45-7.55 (m, 3H), 7.84 (dd, J=8.0, 1.6 Hz, 2H), 8.27 (d, J=2.8 Hz, 1H), 8.58 (d, J=2.8 Hz, 1H), 10.67 (s, 1H).

### EXAMPLE 146—SYNTHESIS OF N-(5-CYANOPYRIDIN-3-YL)-4-CYCLOPROPYL-3-PHENYLISOTHIAZOLE-5-CARBOXAMIDE (I-30)

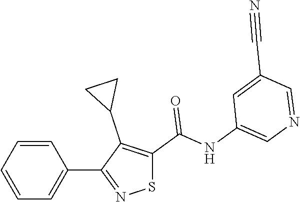 [SMILES: N#Cc1cncc(NC(=O)c2snc(-c3ccccc3)c2C2CC2)c1]

General Procedure A (using 4-cyclopropyl-3-phenylisothiazole-5-carboxylic acid and 5-aminonicotinonitrile yielded the title compound (62 mg, 44%) as a white solid. (ES, m/z): [M+H]⁺ 347.0, ¹H NMR (400 MHz, DMSO-d₆): δ 0.38-0.26 (m, 2H), 0.90-0.79 (m, 2H), 2.15-2.25 (m, 1H), 7.56-7.47 (m, 3H), 7.88-7.80 (m, 2H), 8.60 (t, J=2.0 Hz, 1H), 8.83 (d, J=2.0 Hz, 1H), 9.08 (d, J=2.0 Hz, 1H), 11.18 (s, 1H).

### EXAMPLE 147—SYNTHESIS OF N-(5-CYANO-6-(DIMETHYLAMINO)PYRIDIN-3-YL)-4-CYCLOPROPYL-3-PHENYLISOTHIAZOLE-5-CARBOXAMIDE (I-31)

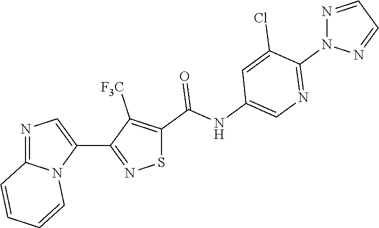 [SMILES: O=C(Nc1cnc(-n2nccn2)c(Cl)c1)c1snc(-c2cnc3ccccn23)c1C(F)(F)F]

General Procedure A (using 3-(imidazo[1,2-a]pyridin-3-yl)-4-(trifluoromethyl)isothiazole-5-carboxylic acid and 5-chloro-6-(2H-1,2,3-triazol-2-yl)pyridin-3-amine) yielded the title compound (3.6 mg, 3%) as an off-white solid. (ES, m/z): [M+H]⁺ 491, ¹H NMR (400 MHz, DMSO-d₆): δ 7.06-7.25 (m, 1H), 7.47-7.56 (m, 1H), 7.80 (d, J=9.0 Hz, 1H), 8.04 (s, 1H), 8.19 (s, 2H), 8.60 (d, J=2.3 Hz, 1H), 8.72 (s, 1H), 9.09-9.46 (m, 1H), 11.94 (s, 1H).

### EXAMPLE 148—SYNTHESIS OF METHYL 2-CYANO-4-(4-CYCLOPROPYL-3-PHENYLISOTHIAZOLE-5-CARBOXAMIDO)BENZOATE

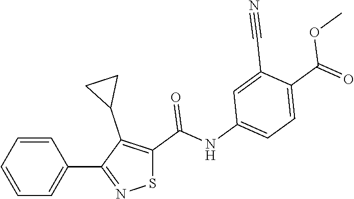 [SMILES: COC(=O)c1ccc(NC(=O)c2snc(-c3ccccc3)c2C2CC2)cc1C#N]

General Procedure A (using 4-cyclopropyl-3-phenylisothiazole-5-carboxylic acid and methyl 4-amino-2-cyanobenzoate) yielded the title compound (279 mg, 85%) as a white solid. (ES, m/z): [M+H]⁺ 404.0, ¹H NMR (400 MHz, DMSO-d₆): δ 0.55-0.65 (m, 2H), 1.11-1.18 (m, 2H), 2.09-2.20 (m, 1H), 4.01 (s, 3H), 7.49 (dd, 0.1=5.2, 2.0 Hz, 3H), 7.70-7.74 (m, 2H), 8.04-8.04 (m, 2H), 8.20 (d, J=8.4 Hz, 1H), 9.01 (s, 1H).

### EXAMPLE 149—SYNTHESIS OF 2-CYANO-4-(4-CYCLOPROPYL-3-PHENYL-ISOTHIAZOLE-5-CARBOXAMIDO)BENZOIC ACID

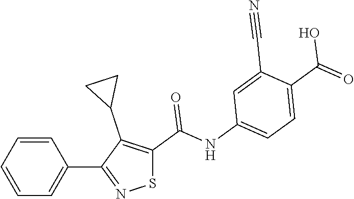 [SMILES: N#Cc1cc(NC(=O)c2snc(-c3ccccc3)c2C2CC2)ccc1C(=O)O]

General Procedure B (using methyl 2-cyano-4-(4-cyclopropyl-3-phenylisothiazole-5-carboxamido)benzoate) yielded the title compound (350 mg, 94%) as a white solid. (ES, m/z): [M+H]⁺ 390.1, ¹H NMR (400 MHz, DMSO-d₆): δ 0.35-0.45 (m, 2H), 0.70-0.80 (m, 2H), 2.15-2.25 (m, 1H), 7.38-7.53 (m, 4H), 7.74 (dd, J=8.4, 1.6 Hz, 2H), 7.84 (d, J=8.4 Hz, 1H), 8.04 (d, J=1.6 Hz, 1H), 9.35 (s, 1H).

### EXAMPLE 150—SYNTHESIS OF N-(4-(3-((TERT-BUTYLDIMETHYLSILYL)OXY) AZETIDINE-1-CARBONYL)-3-CYANOPHENYL)-4-CYCLOPROPYL-3-PHENYL-ISOTHIAZOLE-5-CARBOXAMIDE

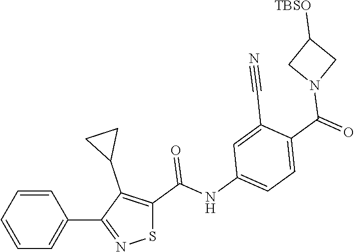 [SMILES: CC(C)(C)[Si](C)(C)OC1CN(C(=O)c2ccc(NC(=O)c3snc(-c4ccccc4)c3C3CC3)cc2C#N)C1]

General Procedure C (using 2-cyano-4-(4-cyclopropyl-3-phenyl-1,2-thiazole-5-amido)benzoic acid and 3-((tert-butyldimethylsilyl)oxy)azetidine) yielded the title compound (253 mg, 42%) as a yellow solid. (ES, m/z): [M+H]⁺ 559.3, ¹H NMR (400 MHz, DMSO-d₆): δ 0.06 (s, 6H), 0.50-0.60 (m, 2H), 0.70-0.80 (m, 2H), 0.89 (s, 9H), 2.15-2.25 (m, 1H), 4.00-4.10 (m, 2H), 4.28-4.35 (m, 1H), 4.40-4.50 (m. 1H), 4.68-4.75 (m, 1H), 7.48 (d, J=5.2 Hz, 2H), 7.59 (d, J=8.4 Hz, 1H), 7.68-7.76 (m, 3H), 7.99 (d, J=8.4 Hz, 1H), 8.04 (s, 1H), 9.02 (s, 1H).

### EXAMPLE 151—SYNTHESIS OF N-(5-CYANOPYRIDIN-3-YL)-4-CYCLOPROPYL-3-PHENYLISOTHIAZOLE-5-CARBOXAMIDE (1-32)

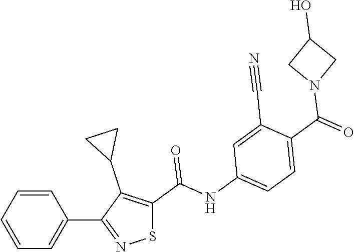 [SMILES: N#Cc1cc(NC(=O)c2snc(-c3ccccc3)c2C2CC2)ccc1C(=O)N1CC(O)C1]

A solution of N-(4-(3-((tert-butyldimethylsilyl)oxy)azetidine-1-carbonyl)-3-cyanophenyl)-4-cyclopropyl-3-phenylisothiazole-5-carboxamide (225 mg, 0.4 mmol, 1 eq.) and CsF (184 mg, 1.2 mmol, 3 eq.) in MeOH (9 mL) was stirred for 3 h at 50° C. under nitrogen. The mixture was filtered and the filtrate was evaporated to dryness. The residue was purified by Prep-HPLC with the following conditions: Column: XBridge Shield RP18 OBD Column, 30*150 mm, m; Mobile Phase A: Water (10 mmol/L NH₄HCO₃), Mobile Phase B: ACN; Flow rate: 60 mL/min; Gradient: 32% B to 57% B in 8 min, 57% B; Wave Length: 220/254 nm; RT1(min): 6.92, resulting in the title compound (61 mg, 34%) as a white solid. (ES, m/z): [M+H]⁺ 445.0, ¹H NMR (400 MHz, DMSO-d₆): δ 0.30-0.40 (m, 2H), 0.85-0.92 (m, 2H), 2.20-2.35 (m, 1H), 3.80-3.90 (m, 1H), 3.95-4.05 (m, 1H), 4.25-4.40 (m, 2H), 4.55-4.65 (m, 1H), 5.83 (d, J=6.0 Hz, 1H), 7.45-7.55 (m, 3H), 7.75 (d, J=8.4 Hz, 1H), 7.84 (dd, J=8.4, 1.2 Hz, 2H), 8.01 (dd, J=8.4, 2.0 Hz, 1H), 8.24 (d, J=2.4 Hz, 1H), 11.11 (s, 1H).

### EXAMPLE 152—SYNTHESIS OF N-(5-CYANO-6-(DIMETHYLAMINO)PYRIDIN-3-YL)-4-CYCLOPROPYL-3-PHENYLISOTHIAZOLE-5-CARBOXAMIDE (1-33)

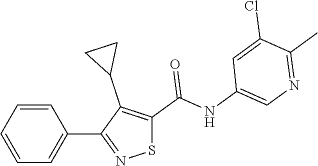 [SMILES: Cc1ncc(NC(=O)c2snc(-c3ccccc3)c2C2CC2)cc1Cl]

General Procedure A (using 4-cyclopropyl-3-phenylisothiazole-5-carboxylic acid and 5-chloro-6-methylpyridin-3-amine) yielded the title compound (23 mg, 15%) as a white solid. (ES, m/z): [M+H]⁺ 370.1, ¹H NMR (400 MHz, DMSO-d₆): δ 0.30-0.40 (m, 2H), 0.85-0.92 (m, 2H), 2.20-2.35 (m, 1H), 2.53 (s, 3H), 7.45-7.55 (m, 3H), 7.80-7.90 (m, 2H), 8.28 (d, J=2.0 Hz, 1H), 8.68 (d, J=2.0 Hz, 1H), 10.94 (s, 1H).

### EXAMPLE 153—SYNTHESIS OF N-(5-CYANOPYRIDIN-3-YL)-4-CYCLOPROPYL-3-PHENYLISOTHIAZOLE-5-CARBOXAMIDE (I-34)

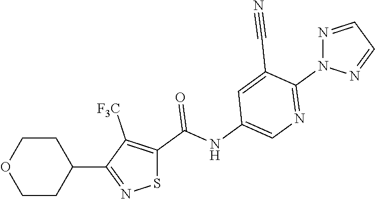 [SMILES: N#Cc1cc(NC(=O)c2snc(C3CCOCC3)c2C(F)(F)F)cnc1-n1nccn1]

General Procedure A (using 3-(tetrahydro-2H-pyran-4-yl)-4-(trifluoromethyl)isothiazole-5-carboxylic acid and 5-amino-2-(2H-1,2,3-triazol-2-yl)nicotinonitrile) yielded the title compound (47 mg, 32%) as a white solid. (ES, m/z): [M+H]⁺ 450.0, ¹H NMR (400 MHz, DMSO-d₆): δ 1.80-1.90 (m, 2H), 2.00-2.15 (m, 2H), 3.20-3.35 (m, 1H), 3.55-3.65 (m, 2H), 4.05-4.20 (m, 2H), 7.95 (s, 1H), 8.04 (s, 2H), 8.78 (d, J=2.4 Hz, 1H), 8.89 (d, J=2.4 Hz, 1H).

### EXAMPLE 154—SYNTHESIS OF N-(5-CHLORO-6-(2H-1,2,3-TRIAZOL-2-YL) PYRIDIN-3-YL)-3-(QUINOLIN-5-YL)-4-(TRIFLUOROMETHYL)ISOTHIAZOLE-5-CARBOXAMIDE (I-35)

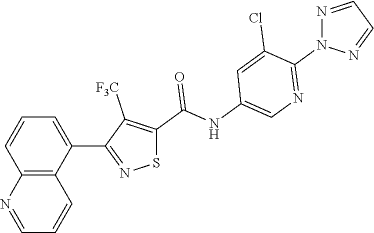 [SMILES: O=C(Nc1cnc(-n2nccn2)c(Cl)c1)c1snc(-c2cccc3ncccc23)c1C(F)(F)F]

General Procedure A (using 3-(quinolin-5-yl)-4-(trifluoromethyl)-1,2-thiazole-5-carboxylic acid and 5-chloro-6-(1,2,3-triazol-2-yl) pyridin-3-amine) yielded the title compound (48 mg, 16%) as a white solid. (ES, m/z): [M+H]⁺ 503.0; ¹H NMR (400 MHz, DMSO-d₆): δ 7.66 (dd, J=8.6, 4.3 Hz, 1H), 7.76 (d, J=7.1 Hz, 1H), 7.99 (td, J=9.0, 7.2 Hz, 2H), 8.19 (s, 2H), 8.27 (d, J=8.5 Hz, 1H), 8.62 (d, J=2.3 Hz, 1H), 8.79 (d, J=2.3 Hz, 1H), 9.01 (dd, J=4.3, 1.6 Hz, 1H).

### EXAMPLE 155—SYNTHESIS OF N-(5-CYANO-6-(2H-1,2,3-TRIAZOL-2-YL)PYRIDIN-3-YL)-3-(1-METHYL-6-OXO-1,6-DIHYDROPYRIDIN-3-YL)-4-(TRIFLUORO-METHYL)ISOTHIAZOLE-5-CARBOXAMIDE (1-36)

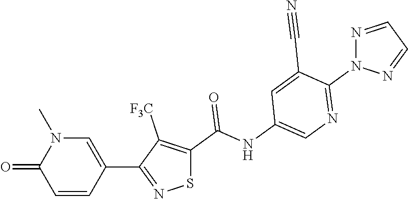 [SMILES: Cn1cc(-c2nsc(C(=O)Nc3cnc(-n4nccn4)c(C#N)c3)c2C(F)(F)F)ccc1=O]

General Procedure A (using 3-(1-methyl-6-oxo-1,6-dihydropyridin-3-yl)-4-(trifluoromethyl) isothiazole-5-carboxylic acid and 5-amino-2-(2H-1,2,3-triazol-2-yl)nicotinonitrile) yielded the title compound (27 mg, 25%) as a white solid. (ES, m/z): [M+H]⁺ 473.0; ¹H NMR (400 MHz, DMSO-d₆): δ 3.53 (s, 3H), 6.51 (d, J=9.6 Hz, 1H), 7.59 (d, J=9.6 Hz, 1H), 8.08 (s, 1H), 8.29 (s, 2H), 8.79 (s, 1H), 8.88 (s, 1H), 11.89 (s, 1H

### EXAMPLE 156—SYNTHESIS OF N-(5-CYANO-6-(2H-1,2,3-TRIAZOL-2-YL)PYRIDIN-3-YL)-3-(1-METHYL-1H-PYRAZOL-4-YL)-4-(TRIFLUORO-METHYL)ISOTHIAZOLE-5-CARBOXAMIDE (I-37)

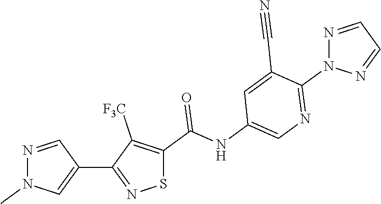 [SMILES: Cn1cc(-c2nsc(C(=O)Nc3cnc(-n4nccn4)c(C#N)c3)c2C(F)(F)F)cn1]

General Procedure A (using 3-(1-methyl-1H-pyrazol-4-yl)-4-(trifluoromethyl)isothiazole-5-carboxylic acid and 5-amino-2-(2H-1,2,3-triazol-2-yl)nicotinonitrile) yielded the title compound (54 mg, 33%) as a white solid. (ES, m/z): [M+H]⁺ 446.0; ¹H NMR (400 MHz, DMSO-d₆): δ 3.94 (s, 3H), 7.85 (s, 1H), 8.21 (s, 1H), 8.32 (s, 2H), 8.75 (d, J=2.5 Hz, 1H), 8.97 (d, J=2.6 Hz, 1H), 11.89 (s, 1H).

### EXAMPLE 157—SYNTHESIS OF N-(5-CYANO-6-(2H-1,2,3-TRIAZOL-2-YL) PYRIDIN-3-YL)-3-ISOPROPYL-4-(TRIFLUOROMETHYL)ISOTHIAZOLE-5-CARBOXAMIDE (I-38)

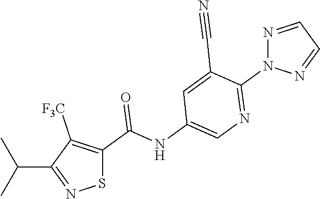 [SMILES: CC(C)c1nsc(C(=O)Nc2cnc(-n3nccn3)c(C#N)c2)c1C(F)(F)F]

General Procedure A (using 3-isopropyl-4-(trifluoromethyl)isothiazole-5-carboxylic acid and 5-amino-2-(2H-1,2,3-triazol-2-yl)nicotinonitrile yielded the title compound (69 mg, 18%) as a white solid. (ES, m/z): [M+H]⁺ 408.0; ¹H NMR (400 MHz, DMSO-d₆): δ 1.37 (d, J=6.8 Hz, 6H), 3.39 (d, J=6.8 Hz, 1H), 8.00-8.05 (m, 3H), 8.78 (d, J=2.4 Hz, 1H), 8.89 (d, J=2.4 Hz, 1H).

### EXAMPLE 158—SYNTHESIS OF N-(5-CHLORO-6-(2H-1,2,3-TRIAZOL-2-YL) PYRIDIN-3-YL)-4-METHOXY-3-PHENYLISOTHIAZOLE-5-CARBOXAMIDE (I-39)

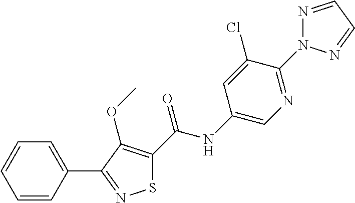 [SMILES: COc1c(-c2ccccc2)nsc1C(=O)Nc1cnc(-n2nccn2)c(Cl)c1]

General Procedure A (using 4-methoxy-3-phenyl-1,2-thiazole-5-carboxylic acid and 5-chloro-6-(1,2,3-triazol-2-yl)pyridin-3-amine) yielded the title compound (6 mg, 3%) as a white solid. (ES, m/z): [M+H]413.0; ¹H NMR (400 MHz, DMSO-d₆): δ 3.94 (s, 3H), 7.38-7.68 (m, 3H), 7.99 (dd, J=8.0, 2.0 Hz, 2H), 8.19 (s, 2H), 8.65 (d, J=2.0 Hz, 1H), 8.86 (d, J=2.4 Hz, 1H), 11.11 (br s, 1H).

### EXAMPLE 159—SYNTHESIS OF N-(5-CHLORO-6-METHOXYPYRIDIN-3-YL)-4-CYCLOPROPYL-3-PHENYLISOTHIAZOLE-5-CARBOXAMIDE (1-40)

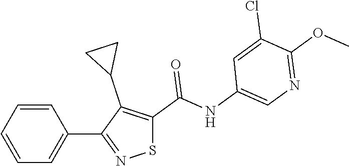 [SMILES: COc1ncc(NC(=O)c2snc(-c3ccccc3)c2C2CC2)cc1Cl]

General Procedure A (using 4-cyclopropyl-3-phenylisothiazole-5-carboxylic acid and 5-chloro-6-methoxypyridin-3-amine) yielded the title compound (9 mg, 4%) as a white solid. (ES, m/z): [M+H]⁺ 386.0, ¹H NMR (400 MHz, DMSO-d₆): δ 0.22-0.40 (m, 2H), 0.78-0.94 (m, 2H), 2.14-2.27 (m, 1H), 3.95 (s, 3H), 7.45-7.59 (m, 3H), 7.78-7.89 (m, 2H), 8.26 (d, J=2.4 Hz, 1H), 8.43 (d, J=2.4 Hz, 1H), 10.79 (s, 1H).

### EXAMPLE 160—SYNTHESIS OF 4-CYCLOPROPYL-N-(1,5-DIMETHYL-6-OXO-1,6-DIHYDROPYRIDIN-3-YL)-3-PHENYLISOTHIAZOLE-5-CARBOXAMIDE (I-41)

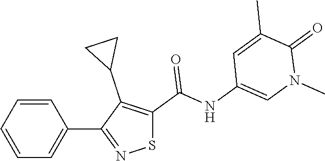 [SMILES: Cc1cc(NC(=O)c2snc(-c3ccccc3)c2C2CC2)cn(C)c1=O]

General Procedure A (using 4-cyclopropyl-3-phenylisothiazole-5-carboxylic acid and 5-amino-1,3-dimethylpyridin-2-one) yielded the title compound (35 mg, 16%) as a white solid. (ES, m/z): [M+H]⁺ 366.1, ¹H NMR (400 MHz, DMSO-d₆): δ 0.28-0.40 (m, 2H), 0.81-0.91 (m, 2H), 2.04 (s, 3H), 2.15-2.25 (m, 1H), 3.48 (s, 3H), 7.44 (dd, J=2.8, 1.2 Hz, 1H), 7.47-7.56 (m, 3H), 7.79-7.85 (m, 2H), 8.09 (d, J=2.8 Hz, 1H), 10.28 (s, 1H).

### EXAMPLE 161—SYNTHESIS OF N-(5-CHLOROPYRIDIN-3-YL)-4-CYCLOPROPYL-3-PHENYLISOTHIAZOLE-5-CARBOXAMIDE (1-42)

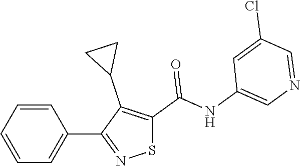 [SMILES: O=C(Nc1cncc(Cl)c1)c1snc(-c2ccccc2)c1C1CC1]

General Procedure A (using 4-cyclopropyl-3-phenylisothiazole-5-carboxylic acid and 5-chloropyridin-3-amine) yielded the title compound (63 mg, 43%) as a white solid. (ES, m/z): [M+H]⁺ 356.0, ¹H NMR (400 MHz, DMSO-d₆): δ 0.20-0.38 (m, 2H), 0.75-0.87 (m, 2H), 2.12-2.23 (m, 1H), 7.45-7.59 (m, 3H), 7.77-7.93 (m, 2H), 8.34 (t, 0.1=2.2 Hz, 1H), 8.45 (d, J=2.2 Hz, 1H), 8.80 (d, J=2.2 Hz, 1H), 11.05 (s, 1H).

### EXAMPLE 162—SYNTHESIS OF 4-CYCLOPROPYL-N-(5-FLUOROPYRIDIN-3-YL)-3-PHENYLISOTHIAZOLE-5-CARBOXAMIDE (1-43)

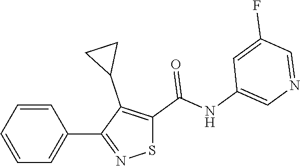 [SMILES: O=C(Nc1cncc(F)c1)c1snc(-c2ccccc2)c1C1CC1]

General Procedure A (using 4-cyclopropyl-3-phenylisothiazole-5-carboxylic acid and 5-fluoropyridin-3-amine) yielded the title compound (64 mg, 31%) as a white solid. (ES, m/z): [M+H]⁺ 340.1, ¹H NMR (400 MHz, DMSO-d₆): δ 0.29-0.37 (m, 2H), 0.79-0.89 (m, 2H), 2.20-2.28 (m, 1H), 7.50-7.58 (m, 3H), 7.80-7.88 (m, 2H), 8.10-8.20 (m, 1H), 8.41 (d, J=2.4 Hz, 1H), 8.71 (t, J=2.4 Hz, 1H), 11.10 (s, 1H).

### EXAMPLE 163—SYNTHESIS OF 4-CYCLOPROPYL-N-(5-METHOXYPYRIDIN-3-YL)-3-PHENYLISOTHIAZOLE-5-CARBOXAMIDE (1-44)

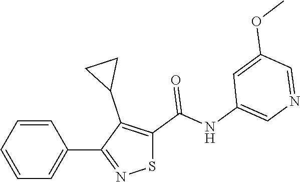 [SMILES: COc1cncc(NC(=O)c2snc(-c3ccccc3)c2C2CC2)c1]

General Procedure A (using 4-cyclopropyl-3-phenylisothiazole-5-carboxylic acid and 5-methoxypyridin-3-amine) yielded the title compound (65 mg, 30%) as a white solid. (ES, m/z): [M+H]⁺ 352.1, ¹H NMR (400 MHz, DMSO-d₆): δ 0.25-0.44 (m, 2H), 0.73-0.96 (m, 2H), 2.20-2.30 (m, 1H), 3.86 (s, 3H), 7.45-7.65 (m, 3H), 7.80-7.90 (m, 3H), 8.13 (d, J=2.4 Hz, 1H), 8.49 (d, J=2.4 Hz, 1H), 10.84 (s, 1H).

### EXAMPLE 164—SYNTHESIS OF 4-CYCLOPROPYL-N-(5-METHYLPYRIDIN-3-YL)-3-PHENYLISOTHIAZOLE-5-CARBOXAMIDE (I-45)

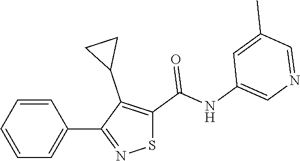 [SMILES: Cc1cncc(NC(=O)c2snc(-c3ccccc3)c2C2CC2)c1]

General Procedure A (using 4-cyclopropyl-3-phenylisothiazole-5-carboxylic acid and 5-methylpyridin-3-amine) yielded the title compound (59 mg, 43%) as a white solid. (ES, m/z): [M+H]⁺ 336.1, ¹H NMR (400 MHz, DMSO-d₆): δ 0.31-0.35 (m, 2H), 0.82-0.86 (M, 2H), 2.19-2.25 (m, 1H), 2.34 (s, 3H), 7.50-7.56 (m, 3H), 7.82-7.86 (m, 2H), 8.00 (t, J=2.0 Hz, 1H), 8.23 (dd, J=2.0, 0.8 Hz, 1H), 8.67 (d, J=2.0 Hz, 1H), 10.76 (s, 1H).

### EXAMPLE 165—SYNTHESIS OF 4-CYCLOPROPYL-N-(6-(DIMETHYLAMINO) PYRIDIN-3-YL)-3-PHENYLISOTHIAZOLE-5-CARBOXAMIDE (I-46)

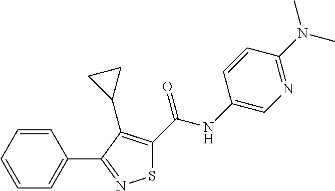 [SMILES: CN(C)c1ccc(NC(=O)c2snc(-c3ccccc3)c2C2CC2)cn1]

General Procedure A (using 4-cyclopropyl-3-phenylisothiazole-5-carboxylic acid and N₂,N₂-dimethylpyridine-2,5-diamine trihydrochloride yielded the title compound (65 mg, 44%) as a white solid. (ES, m/z): [M+H]⁺ 365.1, ¹H NMR (400 MHz, DMSO-d₆): δ 0.32-0.36 (m, 2H), 0.82-0.88 (m, 2H), 2.15-2.25 (m, 1H), 3.02 (s, 6H), 6.69 (d, J=9.2 Hz, 1H), 7.49-7.55 (m, 3H), 7.80-7.88 (m, 3H), 8.37 (d, J=2.4 Hz, 1H), 10.35 (s, 1H).

### EXAMPLE 166—SYNTHESIS OF 4-CYCLOPROPYL-N-(1-METHYL-1H-PYRAZOLO [3,4-B]PYRIDIN-5-YL)-3-PHENYLISOTHIAZOLE-5-CARBOXAMIDE

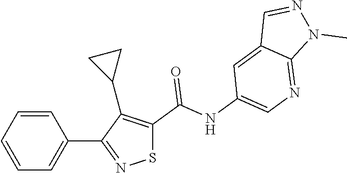 [SMILES: Cn1ncc2cc(NC(=O)c3snc(-c4ccccc4)c3C3CC3)cnc21]

General Procedure A (using 4-cyclopropyl-3-phenylisothiazole-5-carboxylic acid and 1-methylpyrazolo[3,4-b]pyridin-5-amine) yielded the title compound (36 mg, 23%) as a white solid. (ES, m/z): [M+H]⁺ 376.1, ¹H NMR (400 MHz, DMSO-d₆): δ 0.33-0.43 (m, 2H), 0.78-0.88 (m, 2H), 2.20-2.30 (m, 1H), 4.07 (s, 3H), 7.47-7.59 (m, 3H), 7.81-7.93 (m, 2H), 8.18 (s, 1H), 8.66 (d, J=2.4 Hz, 1H), 8.75 (d, J=2.4 Hz, 1H), 10.88 (s, 1H).

### EXAMPLE 167—SYNTHESIS OF 4-CYCLOPROPYL-N-(5-METHYL-6-(1H-1,2,3-TRIAZOL-1-YL)PYRIDIN-3-YL)-3-PHENYLISOTHIAZOLE-5-CARBOXAMIDE (I-48)

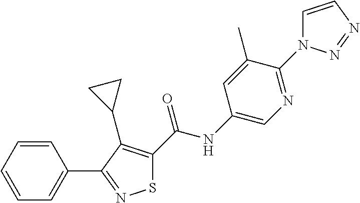 [SMILES: Cc1cc(NC(=O)c2snc(-c3ccccc3)c2C2CC2)cnc1-n1ccnn1]

General Procedure A (using 4-cyclopropyl-3-phenyl-1,2-thiazole-5-carboxylic acid and 5-methyl-6-(1,2,3-triazol-1-yl)pyridin-3-amine) yielded the title compound (63 mg, 18%) as a white solid. (ES, m/z): [M+H]⁺ 403.1; ¹H NMR (400 MHz, DMSO-d₆): δ 0.21-0.51 (M, 2H), 0.72-1.02 (m, 2H), 2.09-2.29 (m, 1H), 2.32 (s, 3H), 7.28-7.68 (m, 3H), 7.72-8.10 (m, 3H), 8.34 (d, J=2.0 Hz, 1H), 8.61 (d, J=0.8 Hz, 1H), 8.76 (d, J=2.0 Hz, 1H), 11.08 (br s, 1H).

### EXAMPLE 168—SYNTHESIS OF N-(3-CHLORO-4-(3-HYDROXYAZETIDINE-1-CARBONYL)PHENYL)-3-(3-CHLOROPYRIDIN-2-YL)-4-CYCLOPROPYL-ISOTHIAZOLE-5-CARBOXAMIDE (1-49)

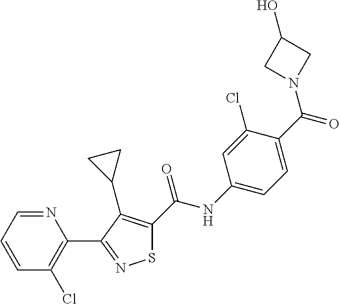 [SMILES: O=C(Nc1ccc(C(=O)N2CC(O)C2)c(Cl)c1)c1snc(-c2ncccc2Cl)c1C1CC1]

General procedure E (using N-(4-(3-((tert-butyldimethylsilyl)oxy)azetidine-1-carbonyl)-3-chlorophenyl)-3-(3-chloropyridin-2-yl)-4-cyclopropylisothiazole-5-carboxamide and CsF) yielded the title compound (24 mg, 37%) as a white solid. (ES, m/z): [M+H]+ 489.1 ¹H NMR (400 MHz, DMSO-d₆,): δ 0.25-0.35 (m, 2H), 0.54-0.67 (m, 2H), 1.95-2.05 (m, 1H), 3.70-3.80 (m, 2H), 4.00-4.10 (m, 1H), 4.20-4.30 (m, 1H), 4.45-4.55 (m, 1H), 5.83 (d, J=6.4 Hz, 1H), 7.45 (d, J=8.4 Hz, 1H), 7.62 (dd, J=8.4, 4.8 Hz, 1H), 7.69 (dd, J=8.4, 2.0 Hz, 1H), 7.95 (d, J=2.0 Hz, 1H), 8.17 (dd, J=8.4, 1.4 Hz, 1H), 8.69 (dd, J=4.8, 1.4 Hz, 1H), 10.96 (s, 1H).

### EXAMPLE 169—SYNTHESIS OF N-(5-CHLORO-6-(DIMETHYLCARBAMOYL) PYRIDIN-3-YL)-4-CYCLOPROPYL-3-PHENYLISOTHIAZOLE-5-CARBOXAMIDE

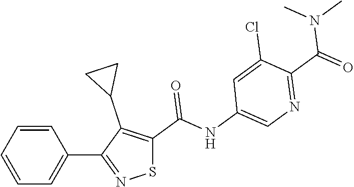 [SMILES: CN(C)C(=O)c1ncc(NC(=O)c2snc(-c3ccccc3)c2C2CC2)cc1Cl]

General procedure A (using 4-cyclopropyl-3-phenyl-1,2-thiazole-5-carboxylic acid and 5-amino-3-chloro-N,N-dimethylpyridine-2-carboxamide) yielded the title compound (29 mg, 5%) as a white solid. (ES, m % z): [M+H]⁺ 427.1; ¹H NMR (DMSO-d₆, 400 MHz, ppm): δ 0.0.30-0.40 (m, 2H), 0.80-0.90 (m, 2H), 2.18-2.25 (m, 1H), 2.80 (s, 3H), 3.03 (s, 3H), 7.47-7.59 (m, 3H), 7.81-7.89 (m, 2H), 8.41 (d, J=2.0 Hz, 1H), 8.81 (d, J=2.0 Hz, 1H), 11.16 (s, 1H).

### EXAMPLE 170—SYNTHESIS OF N-(5-CYANO-6-(N-METHYLACETAMIDO) PYRIDIN-3-YL)-4-CYCLOPROPYL-3-PHENYLISOTHIAZOLE-5-CARBOXAMIDE

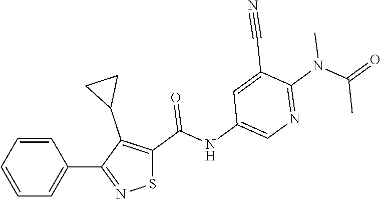 [SMILES: CC(=O)N(C)c1ncc(NC(=O)c2snc(-c3ccccc3)c2C2CC2)cc1C#N]

General procedure A (using 4-cyclopropyl-3-phenyl-1,2-thiazole-5-carboxylic acid and N-(5-amino-3-cyanopyridin-2-yl)-N-methylacetamide) yielded the title compound (69 mg, 31%) as a white solid. (ES, m/z): [M+H]⁺ 418.0; ¹H NMR (DMSO-d₆, 400 MHz, ppm): δ 0.27-0.37 (m, 2H), 0.77-0.94 (m, 2H), 2.05 (s, 3H), 2.18-2.25 (m, 1H), 3.30 (s, 3H), 7.44-7.58 (m, 3H), 7.79-7.90 (m, 2H), 8.67 (s, 1H), 8.99 (d, J=2.6 Hz, 1H), 11.24 (s, 1H).

### EXAMPLE 171—SYNTHESIS OF N-(5-CYANO-6-(2H-1,2,3-TRIAZOL-2-YL) PYRIDIN-3-YL)-4-CYCLOPROPYL-3-HYDROXYISOTHIAZOLE-5-CARBOXAMIDE (I-52)

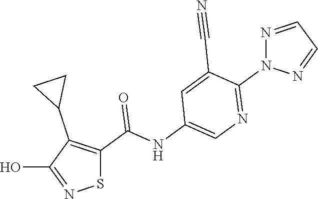 [SMILES: N#Cc1cc(NC(=O)c2snc(O)c2C2CC2)cnc1-n1nccn1]

A solution of 3-(benzyloxy)-N-(5-cyano-6-(2H-1,2,3-triazol-2-yl)pyridin-3-yl)-4-cyclopropyl isothiazole-5-carboxamide (150 mg, 0.3 mmol, 1.0 eq.) in HBr in AcOH (1.5 mL) was stirred for 2 h at room temperature under nitrogen atmosphere. The resulting mixture was diluted with water (10 mL). The precipitate was collected by filtration and rinsed with water (3×5 mL) and EtOAc (3×2 mL) and dried in oven to result in the title compound (66 mg, 55%) as a white solid. (ES, m/z): [M+H]⁺ 354.1; ¹H NMR (DMSO-d₆, 400 MHz, ppm): δ 0.72-0.90 (m, 2H), 0.90-1.10 (m, 2H), 2.10-2.21 (m, 1H), 8.31 (s, 2H), 8.80 (d, J=2.4 Hz, 1H), 9.07 (d, J=2.4 Hz, 1H), 11.16 (br s, 1H), 12.09 (br s, 1H).

### EXAMPLE 172—SYNTHESIS OF N-(5-CYANO-6-MORPHOLINOPYRIDIN-3-YL)-4-CYCLOPROPYL-3-PHENYLISOTHIAZOLE-5-CARBOXAMIDE (1-53)

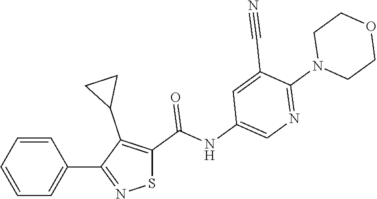 [SMILES: N#Cc1cc(NC(=O)c2snc(-c3ccccc3)c2C2CC2)cnc1N1CCOCC1]

General procedure A (using 4-cyclopropyl-3-phenyl-1,2-thiazole-5-carboxylic acid and 5-amino-2-(morpholin-4-yl)pyridine-3-carbonitrile) yielded the title compound (58 mg, 11%) as a white solid. (ES, m/z): [M+H]⁺ 432.1; ¹H NMR (DMSO-d₆, 400 MHz, ppm): δ 0.28-0.37 (m, 2H), 0.82-0.91 (m, 2H), 2.18-2.25 (m. 1H), 3.52-3.59 (m, 4H), 3.71-3.78 (m, 4H), 7.46-7.58 (m, 3H), 7.80-7.87 (m, 2H), 8.39 (d, J=2.6 Hz, 1H), 8.68 (d, J=2.6 Hz, 1H), 10.81 (s, 1H).

### EXAMPLE 173—SYNTHESIS OF N-(5-CYANO-6-(4-METHYLPIPERAZIN-1-YL) PYRIDIN-3-YL)-4-CYCLOPROPYL-3-PHENYLISOTHIAZOLE-5-CARBOXAMIDE

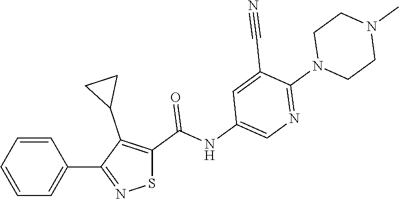 [SMILES: CN1CCN(c2ncc(NC(=O)c3snc(-c4ccccc4)c3C3CC3)cc2C#N)CC1]

General procedure A (using 4-cyclopropyl-3-phenyl-1,2-thiazole-5-carboxylic acid and 5-amino-2-(4-methylpiperazin-1-yl)pyridine-3-carbonitrile) yielded the title compound (8 mg, 2%) as a white solid. (ES, m/z): [M+H]⁺ 445.1; 1H NMR (DMSO-d₆, 400 MHz, ppm): δ 0.29-0.35 (m, 2H), 0.83-0.89 (m, 2H), 2.17-2.20 (m, 1H), 2.23 (s, 3H), 2.40-2.50 (m, 4H), 3.50-3.62 (m, 4H), 7.48-7.56 (m, 3H), 7.83 (dd, J=8.0, 1.8 Hz, 2H), 8.36 (d. J=2.8 Hz, 1H), 8.65 (d. J=2.8 Hz, 1H), 10.78 (s, 1H).

### EXAMPLE 174—SYNTHESIS OF 4-CYCLOPROPYL-N-(4-(DIMETHYL-CARBAMOYL)-3-(TRIFLUOROMETHYL) PHENYL)-3-PHENYLISOTHIAZOLE-5-CARBOXAMIDE (I-55)

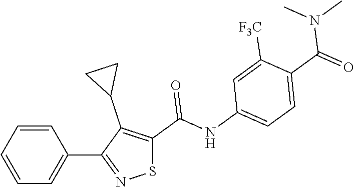 [SMILES: CN(C)C(=O)c1ccc(NC(=O)c2snc(-c3ccccc3)c2C2CC2)cc1C(F)(F)F]

General procedure A (using 4-cyclopropyl-3-phenyl-1.2-thiazole-5-carboxylic acid and 4-amino-N,N-dimethyl-2-(trifluoromethyl)benzamide) yielded the title compound (77 mg, 40%) as a white solid. (ES, m/z): [M+H]⁺ 460.1; ¹H NMR (DMSO-d₆, 400 MHz, ppm): δ 0.29-0.38 (m, 2H), 0.78-0.88 (m, 2H), 2.15-2.30 (m, 1H), 2.76 (s, 3H), 3.00 (s, 3H), 7.42-7.56 (m, 4H), 7.79-7.87 (m, 2H), 8.00 (dd, J=8.4, 2.0 Hz, 1H), 8.23 (d, J=2.0 Hz, 1H), 11.04 (s, 1H).

### EXAMPLE 175—SYNTHESIS OF N-(5-CYANO-6-(2H-1,2,3-TRIAZOL-2-YL) PYRIDIN-3-YL)-4-CYCLOPROPYL-3-ISOPROPYLISOTHIAZOLE-5-CARBOXAMIDE (I-56)

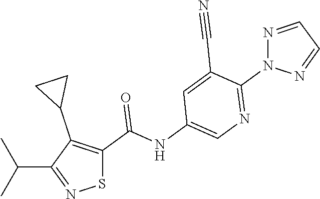 [SMILES: CC(C)c1nsc(C(=O)Nc2cnc(-n3nccn3)c(C#N)c2)c1C1CC1]

General procedure A (using methyl 4-cyclopropyl-3-isopropylisothiazole-5-carboxylate and 5-amino-2-(2H-1,2,3-triazol-2-yl)nicotinonitrile) yielded the title compound (14 mg, 8%) as an off-white solid. (ES, m/z): [M+H]⁺ 380; ¹H NMR (DMSO-d₆, 400 MHz, ppm): δ 0.47-0.63 (m, 2H), 0.88-1.04 (m, 2H), 1.31 (d, J=6.8 Hz, 6H), 1.90-2.02 (m, 1H), 3.45-3.55 (m, 1H), 8.32 (s, 2H), 8.81 (d, J=2.5 Hz, 1H), 9.06 (d, J=2.5 Hz, 1H), 11.28 (s, 1H).

### EXAMPLE 176—SYNTHESIS OF N-(5-CHLORO-6-(2H-1,2,3-TRIAZOL-2-YL) PYRIDIN-3-YL)-5-PHENYL-4-(TRIFLUOROMETHYL)ISOTHIAZOLE-3-CARBOXAMIDE (I-57)

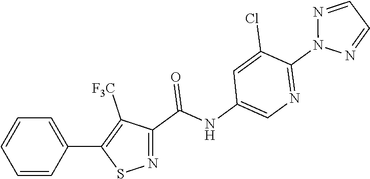 [SMILES: O=C(Nc1cnc(-n2nccn2)c(Cl)c1)c1nsc(-c2ccccc2)c1C(F)(F)F]

A small variation of General Procedure A (using 5-phenyl-4-(trifluoromethyl)isothiazole-3-carboxylic acid and 5-chloro-6-(1,2,3-triazol-1-yl)pyridin-3-amine) yielded the title compound (63 mg, 18%) as a white solid. (ES, m/z): [M+H]⁺ 403.1; ¹H NMR (400 MHz, DMSO-d₆): δ 0.21-0.51 (m, 2H), 0.72-1.02 (m, 2H), 2.09-2.29 (m, 1H), 2.32 (s, 3H), 7.28-7.68 (m, 3H), 7.72-8.10 (m, 3H), 8.34 (d, J=2.0 Hz, 1H), 8.61 (d, J=0.8 Hz, 1H), 8.76 (d, J=2.0 Hz, 1H), 11.08 (br s, 1H).

### EXAMPLE 177—SYNTHESIS OF N-(5-CHLORO-6-(3-HYDROXYAZETIDINE-1-CARBONYL)PYRIDIN-3-YL)-4-CYCLOPROPYL-3-PHENYLISOTHIAZOLE-5-CARBOXAMIDE (I-58)

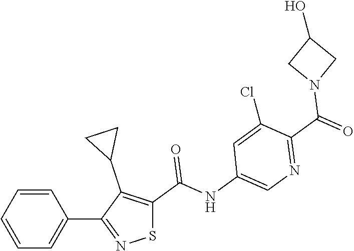 [SMILES: O=C(Nc1cnc(C(=O)N2CC(O)C2)c(Cl)c1)c1snc(-c2ccccc2)c1C1CC1]

General procedure E (using N-(6-(3-((tert-butyldimethylsilyl)oxy)azetidine-1-carbonyl)-5-chloropyridin-3-yl)-4-cyclopropyl-3-phenylisothiazole-5-carboxamide and CsF) yielded the title compound (27 mg, 42%) as a white solid. (ES, m/z): [M+H]⁺ 455; ¹H NMR (DMSO-d₆, 400 MHz, ppm): δ 0.24-0.42 (m, 2H), 0.79-0.93 (m, 2H), 2, 14-2.32 (m, 1H), 3.73-3.88 (m, 2H), 4.12-4.31 (m, 2H), 4.46-4.62 (m, 1H), 5.82 (d, J=6.4 Hz, 1H), 7.43-7.59 (m, 3H), 7.78-7.87 (m, 2H), 8.40 (d, J=2.0 Hz, 1H), 8.80 (d, J=2.0 Hz, 1H), 11.14 (s, 1H).

### EXAMPLE 178—SYNTHESIS OF 4-CYCLOPROPYL-N-(6-(3-HYDROXY-AZETIDINE-1-CARBONYL)PYRIDIN-3-YL)-3-PHENYLISOTHIAZOLE-5-CARBOXAMIDE (I-59)

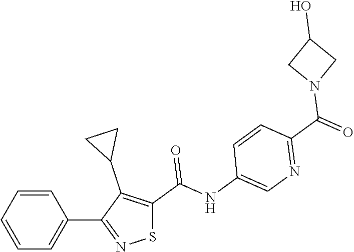 [SMILES: O=C(Nc1ccc(C(=O)N2CC(O)C2)nc1)c1snc(-c2ccccc2)c1C1CC1]

General procedure E (using N-(6-(3-((tert-butyldimethylsilyl)oxy)azetidine-1-carbonyl)pyridin-3-yl)-4-cyclopropyl-3-phenylisothiazole-5-carboxamide and CsF) yielded the title compound (41 mg, 86%) as a white solid. (ES, m/z): [M+H]⁺ 421; ¹H NMR (DMSO-d₆, 400 MHz, ppm): δ 0.30-0.40 (m, 2H), 0.74-0.85 (m, 2H), 2.20-2.30 (m, 1H), 3.75-3.85 (m, 1H), 4.25-4.32 (m, 2H), 4.49-4.52 (m, 1H), 4.70-4.80 (m, 1H), 5.72 (d, J=6.4 Hz, 1H), 7.51-7.56 (m, 3H), 7.84 (dd, J=7.6, 2.0 Hz, 2H), 8.01 (d, J=8.8 Hz, 1H), 8.29 (dd, J=8.4, 2.4 Hz, 1H), 8.90 (d, J=2.4 Hz, 1H), 11.08 (s, 1H).

### EXAMPLE 179—SYNTHESIS OF N-(5-CHLORO-6-(2H-1,2,3-TRIAZOL-2-YL) PYRIDIN-3-YL)-4-CYCLOPROPYL-3-ISOPROPYLISOTHIAZOLE-5-CARBOXAMIDE (I-60)

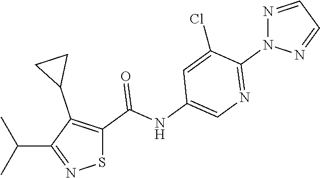 [SMILES: CC(C)c1nsc(C(=O)Nc2cnc(-n3nccn3)c(Cl)c2)c1C1CC1]

General procedure D (using methyl 4-cyclopropyl-3-isopropylisothiazole-5-carboxylate and 5-chloro-6-(2H-1,2,3-triazol-2-yl)pyridin-3-amine) yielded the title compound (44 mg, 10%) as an off-white solid. (ES, m/z): [M+H]⁺ 389; ¹H NMR (DMSO-d₆, 400 MHz, ppm): δ 0.54-0.59 (m, 2H), 0.95-1.01 (m, 2H), 1.31 (d, J=6.8 Hz, 6H), 1.94-2.02 (m, 1H), 3.43-3.49 (m, 1H), 8.19 (s, 2H), 8.61 (d, J=2.2 Hz, 1H), 8.81 (d, J=2.2 Hz, 1H), 11.25 (s, 1H).

### EXAMPLE 180—SYNTHESIS OF 4-CYCLOPROPYL-N-(6-(3-HYDROXY-AZETIDINE-1-CARBONYL)PYRIDIN-3-YL)-3-PHENYLISOTHIAZOLE-5-CARBOXAMIDE (I-61)

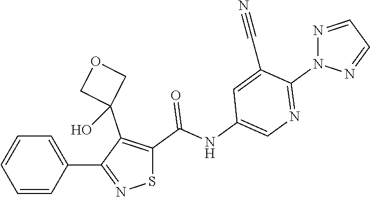 [SMILES: N#Cc1cc(NC(=O)c2snc(-c3ccccc3)c2C2(O)COC2)cnc1-n1nccn1]

General procedure D (using methyl 4-(3-hydroxyoxetan-3-yl)-3-phenylisothiazole-5-carboxylate and 5-amino-2-(2H-1,2,3-triazol-2-yl)nicotinonitrile) yielded the title compound (12 mg, 6%) as a white solid. (ES, m/z): [M+H]⁺ 446.1; ¹H NMR (DMSO-d₆, 400 MHz, ppm): δ 3.43-3.59 (m, 1H), 3.61-3.81 (m, 2H), 4.05 (d, J=12.8 Hz, 1H), 4.46 (d, J=13.2 Hz, 1H), 4.92-5.18 (m, 1H), 6.37 (s, 1H), 7.38-7.62 (m, 3H), 7.70-7.93 (m, 2H), 8.70 (d, J=2.4 Hz, 1H), 9.04 (d, J=2.8 Hz, 1H).

### EXAMPLE 181—SYNTHESIS OF 4-CYCLOPROPYL-N-(2-METHYL-1-OXO-1,2-DIHYDROISOQUINOLIN-6-YL)-3-PHENYLISOTHIAZOLE-5-CARBOXAMIDE

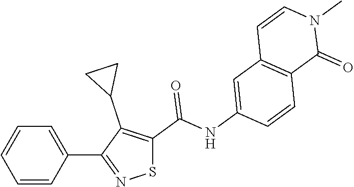 [SMILES: Cn1ccc2cc(NC(=O)c3snc(-c4ccccc4)c3C3CC3)ccc2c1=O]

General procedure A (using 4-cyclopropyl-3-phenyl-1.2-thiazole-5-carboxylic acid and 6-amino-2-methylisoquinolin-1(2H)-one yielded the title compound (6 mg, 4%) as a white solid. (ES, m/z): [M+H]⁺ 402.1; ¹H NMR (400 MHz, DMSO-d₆): δ 0.28-0.46 (m, 2H), 0.78-0.94 (m, 2H), 2.21-2.31 (m, 1H), 3.50 (s, 3H), 6.62 (d, J=7.2 Hz, 1H), 7.38-7.59 (m, 4H), 7.69 (dd, J=8.8, 2.0 Hz, 1H), 7.81-7.91 (m, 2H), 8.13 (d, J=2.0 Hz, 1H), 8.22 (d, J=8.8 Hz, 1H), 10.97 (s, 1H).

### EXAMPLE 182—SYNTHESIS OF N-(5-CHLORO-6-(2H-1,2,3-TRIAZOL-2-YL) PYRIDIN-3-YL)-3-(2-OXO-1,2-DIHYDROQUINOLIN-5-YL)-4-(TRIFLUORO-METHYL)ISOTHIAZOLE-5-CARBOXAMIDE

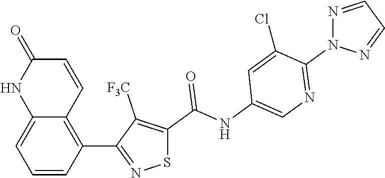 [SMILES: O=C(Nc1cnc(-n2nccn2)c(Cl)c1)c1snc(-c2cccc3[nH]c(=O)ccc23)c1C(F)(F)F]

To a stirred solution of N-[5-chloro-6-(1,2,3-triazol-2-yl)pyridin-3-yl]-3-(2-methoxyquinolin-5-yl)-4-(trifluoromethyl)-1,2-thiazole-5-carboxamide (60 mg, 0.2 mmol, 1.0 eq.) in HOAc (2 mL) was added HBr in AcOH (40%, 23 mg, 0.3 mmol, 1.5 eq.) dropwise at 0° C. under nitrogen. The resulting mixture was stirred for 30 min at room temperature under nitrogen. The resulting mixture was poured into water (20 mL) and extracted with EtOAc (2×20 mL). The combined organic layers were washed with brine (2×50 mL), dried over anhydrous Na₂SO₄, and concentrated under reduced pressure. The residue was purified by reversed-phase flash chromatography with the following conditions: column, C18 silica gel; mobile phase, MeCN in Water (0.1% FA), 50% to 70% gradient in 10 min; detector, UV 254 nm. This resulted in the title compound (19 mg, 33%) as a white solid. (ES, m/z): [M+H]⁺ 518; ¹H NMR (400 MHz, DMSO-d₆) δ 6.68 (d, J=10.0 Hz, 1H), 7.23 (d, J=7.2 Hz, 1H), 7.42 (d, J=9.6 Hz, 1H), 7.51 (d, J=4.4 Hz, 1H), 7.61-7.71 (m, 1H), 8.20 (s, 2H), 8.60 (d, J=2.4 Hz, 1H), 8.77 (d, J=2.0 Hz, 1H), 11.87 (s, 1H), 12.04 (s, 1H).

### EXAMPLE 183—SYNTHESIS OF 3-(BENZO[D]THIAZOL-7-YL)-N-(5-CYANO-6-(2H-1,2,3-TRIAZOL-2-YL)PYRIDIN-3-YL)-4-CYCLOPROPYLISOTHIAZOLE-5-CARBOXAMIDE

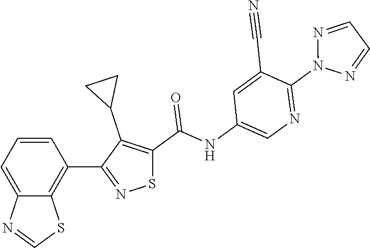 [SMILES: N#Cc1cc(NC(=O)c2snc(-c3cccc4ncsc34)c2C2CC2)cnc1-n1nccn1]

General procedure A (using 3-(benzo[d]thiazol-7-yl)-4-cyclopropylisothiazole-5-carboxylic acid and 5-amino-2-(2H-1,2,3-triazol-2-yl)nicotinonitrile) yielded the title compound (35 mg, 26%) as an off-white solid. (ES, m/z): [M+H]⁺ 471; ¹H NMR (400 MHz, DMSO-d₆): δ 0.31-0.42 (m, 2H), 0.86-1.01 (m, 2H), 2.22-2.38 (m, 1H), 7.77 (t, J=8.0 Hz, 1H), 8.25 (dd, J=8.0, 2.8 Hz, 2H), 8.33 (s, 2H), 8.86 (d, J=2.4 Hz, 1H), 9.10 (d. J=2.4 Hz, 1H), 9.50 (s, 1H), 11.51 (s, 1H).

### EXAMPLE 184—SYNTHESIS OF N-(5-CYANO-6-(2H-1,2,3-TRIAZOL-2-YL) PYRIDIN-3-YL)-4-CYCLOPROPYL-3-(1-METHYL-1H-INDAZOL-7-YL) ISOTHIAZOLE-5-CARBOXAMIDE

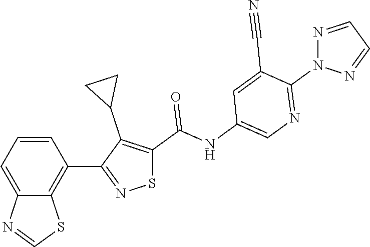 [SMILES: N#Cc1cc(NC(=O)c2snc(-c3cccc4ncsc34)c2C2CC2)cnc1-n1nccn1]

General procedure D (using ethyl 4-cyclopropyl-3-(1-methyl-1H-indazol-7-yl)isothiazole-5-carboxylate and 5-amino-2-(1,2,3-triazol-2-yl)pyridine-3-carbonitrile) yielded the title compound (30 mg, 16%) as a white solid. (ES, m/z): [M+H]⁺ 468; ¹H NMR (400 MHz, CDCl₃): δ 0.55-0.63 (m, 2H), 0.84-0.95 (m, 2H), 1.98-2.11 (m, 1H), 3.76 (s, 3H), 7.32 (dd, J=8.0, 7.2 Hz, 1H), 7.44 (dd, J=7.2, 1.2 Hz, 1H), 7.92 (dd, J=8.0, 1.2 Hz, 1H), 8.04 (s, 2H), 8.20 (s, 1H), 8.94-9.04 (m, 2H), 9.35 (s, 1H).

### EXAMPLE 185—SYNTHESIS OF N-(5-CYANO-6-(2H-1,2,3-TRIAZOL-2-YL) PYRIDIN-3-YL)-3-(DIMETHYLAMINO)-4-(TRIFLUOROMETHYL)ISOTHIAZOLE-5-CARBOXAMIDE

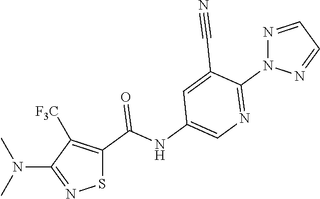 [SMILES: CN(C)c1nsc(C(=O)Nc2cnc(-n3nccn3)c(C#N)c2)c1C(F)(F)F]

General procedure D (using ethyl 3-(dimethylamino)-4-(trifluoromethyl)-1,2-thiazole-5-carboxylate and 5-amino-2-(1,2,3-triazol-2-yl)pyridine-3-carbonitrile) yielded the title compound (3 mg, 10%) as a white solid. (ES, m/z): [M+H]⁺ 409; ¹H NMR (400 MHz, DMSO-d₆): S 2.95 (s, 6H), 8.31 (s, 2H), 8.74 (d, J=2.5 Hz, 1H), 8.98 (d, J=2.5 Hz, 1H), 11.77 (s, 1H).

### EXAMPLE 186—SYNTHESIS OF 3-(BENZO[D]THIAZOL-7-YL)-4-CYCLOPROPYL-N-(2-(TRIFLUOROMETHYL)PYRIDIN-4-YL)ISOTHIAZOLE-5-CARBOXAMIDE

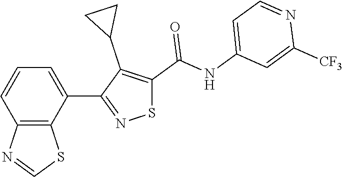 [SMILES: O=C(Nc1ccnc(C(F)(F)F)c1)c1snc(-c2cccc3ncsc23)c1C1CC1]

General procedure D (using methyl 3-(benzo[d]thiazol-7-yl)-4-cyclopropylisothiazole-5-carboxylate and 2-(trifluoromethyl)pyridin-4-amine) yielded the title compound (28 mg, 20%) as a white solid. (ES, m/z): [M+H]⁺ 447.0; ¹H NMR (400 MHz, DMSO-d₆): δ 0.25-0.47 (m, 2H), 0.79-1.02 (m, 2H), 2.09-2.29 (m, 1H), 3.35 (s, 3H), 7.76 (d, J=8.0 Hz, 1H), 7.88-8.08 (m, 1H), 8.15-8.38 (m, 3H), 8.75 (d, J=5.2 Hz, 1H), 9.50 (s, 1H), 11.50 (s, 1H).

### EXAMPLE 187—SYNTHESIS OF 3-(BENZO[D]THIAZOL-7-YL)-4-CYCLOPROPYL-N-(2-METHYLPYRIDIN-4-YL)ISOTHIAZOLE-5-CARBOXAMIDE

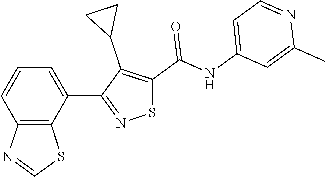 [SMILES: Cc1cc(NC(=O)c2snc(-c3cccc4ncsc34)c2C2CC2)ccn1]

General procedure D (using methyl 3-(benzo[d]thiazol-7-yl)-4-cyclopropylisothiazole-5-carboxylate and 2-methylpyridin-4-amine) yielded the title compound (24 mg, 20%) as a white solid. (ES, m/z): [M+H]⁺ 393.0; ¹H NMR (400 MHz, DMSO-d₆): δ 0.30-0.37 (m, 2H), 0.84-0.92 (m, 2H), 2.28-2.34 (m, 1H), 2.47 (s, 3H), 7.50 (dd, J=5.2, 2.0 Hz, 1H), 7.59 (d, J=2.0 Hz, 1H), 7.76 (t, J=8.0 Hz, 1H), 8.18-8.27 (m, 2H), 8.40 (d, J=5.6 Hz, 1H), 9.49 (s, 1H), 11.04 (s, 1H).

### EXAMPLE 188—SYNTHESIS OF N-(5-CHLORO-6-(2H-1,2,3-TRIAZOL-2-YL) PYRIDIN-3-YL)-3-(2-METHOXYQUINOLIN-5-YL)-4-(TRIFLUOROMETHYL) ISOTHIAZOLE-5-CARBOXAMIDE

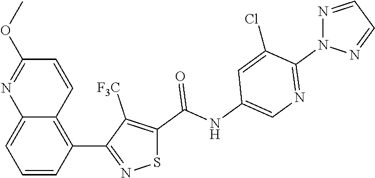 [SMILES: COc1ccc2c(-c3nsc(C(=O)Nc4cnc(-n5nccn5)c(Cl)c4)c3C(F)(F)F)cccc2n1]

General procedure D (using ethyl 3-(2-methoxyquinolin-5-yl)-4-(trifluoromethyl)-1,2-thiazole-5-carboxylate and 5-chloro-6-(1,2,3-triazol-2-yl)pyridin-3-amine) yielded the title compound (70 mg, 46%) as a white solid. (ES, m/z): [M+H]⁺ 532; ¹H NMR (400 MHz, DMSO-d_{b}): δ 4.03 (s, 3H), 7.11 (d, J=9.2 Hz, 1H), 7.52 (d, J=6.8 Hz, 1H), 7.74-7.86 (m, 2H), 7.99 (d, J=8.4 Hz, 1H), 8.21 (br s, 2H), 8.20 (s, 1H), 8.61 (d, J=2.0 Hz, 1H), 8.78 (d, J=2.4 Hz, 1H), 11.90 (s, 1H).

### EXAMPLE 189—SYNTHESIS OF N-(5-CHLORO-6-(2H-1,2,3-TRIAZOL-2-YL) PYRIDIN-3-YL)-3-(1-METHOXYISOQUINOLIN-5-YL)-4-(TRIFLUOROMETHYL) ISOTHIAZOLE-5-CARBOXAMIDE

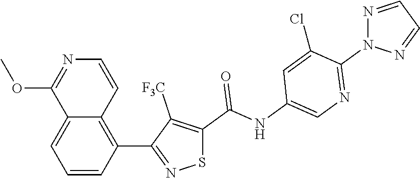 [SMILES: COc1nccc2c(-c3nsc(C(=O)Nc4cnc(-n5nccn5)c(Cl)c4)c3C(F)(F)F)cccc12]

General procedure D (using ethyl 3-(1-methoxyisoquinolin-5-yl)-4-(trifluoromethyl)-1,2-thiazole-5-carboxylate and 5-chloro-6-(1,2,3-triazol-2-yl)pyridin-3-amine) yielded the title compound (7 mg, 48%) as a white solid. (ES, m/z): [M+H]⁺ 532; ¹H NMR (400 MHz, DMSO-d₆): δ 4.11 (s, 3H), 6.88 (d, J=6.0 Hz, 1H), 7.72-7.92 (m, 2H), 8.07 (d, J=6.0 Hz, 1H), 8.20 (s, 2H), 8.35-8.45 (m, 1H), 8.61 (d, J=2.2 Hz, 1H), 8.76 (d, J=2.2 Hz, 1H), 11.88 (s, 1H).

### EXAMPLE 190—SYNTHESIS OF N-(5-CHLORO-6-(2H-1,2,3-TRIAZOL-2-YL) PYRIDIN-3-YL)-3-(1-OXO-1,2-DIHYDROISOQUINOLIN-5-YL)-4-(TRIFLUORO-METHYL)ISOTHIAZOLE-5-CARBOXAMIDE

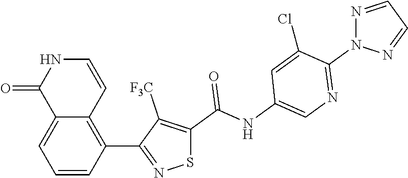 [SMILES: O=C(Nc1cnc(-n2nccn2)c(Cl)c1)c1snc(-c2cccc3c(=O)[nH]ccc23)c1C(F)(F)F]

To a stirred mixture of N-(5-chloro-6-(2H-1,2,3-triazol-2-yl)pyridin-3-yl)-3-(1-methoxyisoquinolin-5-yl)-4-(trifluoromethyl)isothiazole-5-carboxamide (30 mg, 0.06 mmol, 1 eq.) in HOAc (3 mL) was added HBr in AcOH (0.6 mL, 33%) at room temperature. The resulting mixture was stirred for 3 h at 50° C. and evaporated to dryness. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm. This resulted in the title compound (21 mg, 71%) as a white solid. (ES, m/z): [M+H]⁺ 518, ¹H NMR (400 MHz, Methanol-d₄): δ 6.24 (d, J=7.5 Hz, 1H), 7.19 (d, J=7.4 Hz, 1H), 7.64 (t, J=7.8 Hz, 1H), 7.75 (dd, J=7.4, 1.4 Hz, 1H), 8.03 (s, 2H), 8.50 (dt, J=8.1, 1.1 Hz, 1H), 8.62 (d, J=2.3 Hz, 1H), 8.68 (d, J=2.3 Hz, 1H).

### EXAMPLE 191—SYNTHESIS OF N-(5-CYANO-6-(2H-1,2,3-TRIAZOL-2-YL)PYRIDIN-3-YL)-3-(1-METHYL-2-OXO-1,2-DIHYDROPYRIDIN-3-YL)-4-(TRIFLUOROMETHYL)ISOTHIAZOLE-5-CARBOXAMIDE

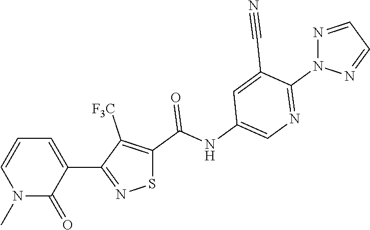 [SMILES: Cn1cccc(-c2nsc(C(=O)Nc3cnc(-n4nccn4)c(C#N)c3)c2C(F)(F)F)c1=O]

General procedure A (using 3-(1-methyl-2-oxo-1,2-dihydropyridin-3-yl)-4-(trifluoromethyl) isothiazole-5-carboxylic acid and 5-amino-2-(2H-1,2,3-triazol-2-yl) nicotinonitrile) yielded the title compound (18 mg, 23%) as a white solid. (ES, m/z): [M+H]⁺ 473; ¹H NMR (400 MHz, DMSO-d₆): δ 3.52 (s, 3H), 6.38 (t, J=6.8 Hz, 1H), 7.62 (dd, J=6.8, 2.0 Hz, 1H), 7.91 (dd. J=6.8, 2.0 Hz, 1H), 8.32 (s, 2H), 8.78 (d, J=2.4 Hz, 1H), 9.01 (d, J=2.4 Hz, 1H), 11.91 (s, 1H).

### EXAMPLE 192—SYNTHESIS OF 3-(BENZO[D]OXAZOL-7-YL)-N-(5-CYANO-6-(2H-1,2,3-TRIAZOL-2-YL)PYRIDIN-3-YL)-4-CYCLOPROPYLISOTHIAZOLE-5-CARBOXAMIDE

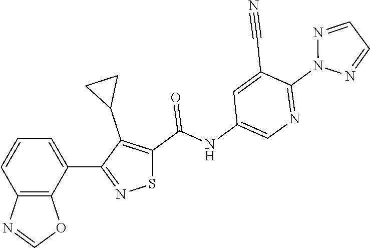 [SMILES: N#Cc1cc(NC(=O)c2snc(-c3cccc4ncoc34)c2C2CC2)cnc1-n1nccn1]

General procedure H (using 5-((5-cyano-6-(2H-1,2,3-triazol-2-yl)pyridin-3-yl)carbamoyl)-4-cyclopropylisothiazol-3-yl trifluoromethanesulfonate and 7-(4,4,5,5-tetramethyl-1,3,2-dioxaborolan-2-yl)benzo[d]oxazole) yielded the title compound (2 mg, 4%) as a white solid. (ES, m/z): [M+H]⁺ 455; ¹H NMR (400 MHz, DMSO-d₆): δ 0.17-0.40 (m, 2H), 0.58-0.72 (d, J=8.4 Hz, 2H), 2.10-2.28 (m, 1H), 7.57 (t, J=8.0 Hz, 1H), 7.72 (d, J=7.6 Hz, 1H), 7.97 (d, J=8.0 Hz, 1H), 8.31 (s, 2H), 8.80-8.92 (m, 2H), 9.05 (br s, 1H), 11.41 (br s, 1H).

### EXAMPLE 193—SYNTHESIS OF N-[5-CYANO-6-(1,2,3-TRIAZOL-2-YL)PYRIDIN-3-YL]-4-CYCLOPROPYL-3-(OXOLAN-3-YL)-1,2-THIAZOLE-5-CARBOXAMIDE

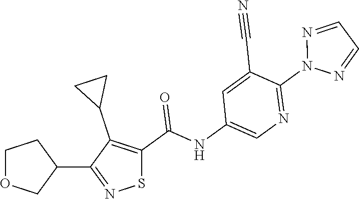 [SMILES: N#Cc1cc(NC(=O)c2snc(C3CCOC3)c2C2CC2)cnc1-n1nccn1]

General procedure D (using ethyl 4-cyclopropyl-3-(tetrahydrofuran-3-yl)isothiazole-5-carboxylate and 5-amino-2-(1,2,3-triazol-2-yl)pyridine-3-carbonitrile (31 mg, 24%) as a white solid. (ES, m/z): [M+H]⁺ 408; ¹H NMR (400 MHz, CDCl₃,): δ 8.94 (d, J=2.6 Hz, 1H), 8.82 (d, J=2.6 Hz, 1H), 8.62 (s, 1H), 8.03 (s, 2H), 4.15-4.27 (m, 1H), 4.05-4.15 (m, 1H), 3.86-4.05 (m, 3H), 2.30-2.40 (m, 2H), 1.90-2.00 (m, 1H), 1.25-1.38 (m, 2H), 0.80-0.90 (m, 2H).

### EXAMPLE 194—SYNTHESIS OF 4-CYCLOPROPYL-N-(6-METHYL-7-OXO-6,7-DIHYDRO-5H-PYRROLO[3,4-B]PYRIDIN-3-YL)-3-PHENYLISOTHIAZOLE-5-CARBOXAMIDE

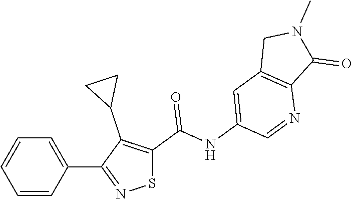 [SMILES: CN1Cc2cc(NC(=O)c3snc(-c4ccccc4)c3C3CC3)cnc2C1=O]

General procedure D (using methyl 4-cyclopropyl-3-phenylisothiazole-5-carboxylate and 3-amino-6-methyl-5,6-dihydro-7H-pyrrolo[3,4-b]pyridin-7-one) (17 mg, 7%) as a white solid. (ES, m/z): [M+H]⁺ 391; ¹H NMR (400 MHz, DMSO-d₆): δ 11.16 (s, 1H), 8.87 (d, J=2.4 Hz, 1H), 8.54 (d, J=2.4 Hz, 1H), 7.80-7.91 (m, 2H), 7.46-7.62 (m, 3H), 4.52 (s, 2H), 3.11 (s, 3H), 2.20-2.30 (m, 1H), 0.74-0.95 (m, 2H), 0.30-0.40 (m, 2H).

### EXAMPLE 195—SYNTHESIS OF N-(5-CYANO-6-(2H-1,2,3-TRIAZOL-2-YL) PYRIDIN-3-YL)-4-CYCLOPROPYL-3-(3-METHYLPYRIDIN-2-YL)ISOTHIAZOLE-5-CARBOXAMIDE

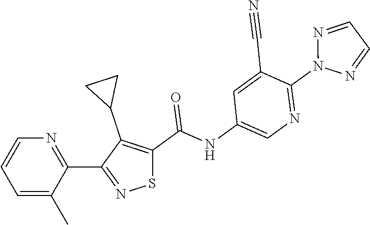 [SMILES: Cc1cccnc1-c1nsc(C(=O)Nc2cnc(-n3nccn3)c(C#N)c2)c1C1CC1]

General procedure H (using 5-((5-cyano-6-(2H-1,2,3-triazol-2-yl)pyridin-3-yl)carbamoyl)-4-cyclopropylisothiazol-3-yl trifluoromethanesulfonate and 3-methyl-2-(tributylstannyl)pyridine) yielded the title compound (0.5 mg, 0.7%) as a white solid. (ES, m/z): [M+H]⁺ 429; ¹H NMR (400 MHz, DMSO-d₆): δ 0.21-0.44 (m, 2H), 0.51-0.77 (m, 2H), 2.08-2.20 (m, 1H), 2.24 (s, 3H), 7.43 (dd, J=4.8, 0.8 Hz, 1H), 7.83 (d, J=8.0 Hz, 1H), 8.31 (s, 2H), 8.53 (d, J=4.8 Hz, 1H), 8.55 (d, J=2.4 Hz, 1H) 9.07 (br s, 1H), 11.39 (br s, 1H).

### EXAMPLE 196—SYNTHESIS OF 3-(BENZO[D]THIAZOL-7-YL)-4-CYCLOPROPYL-N-(5-METHYL-6-(2H-1,2,3-TRIAZOL-2-YL)PYRIDIN-3-YL)ISOTHIAZOLE-5-CARBOXAMIDE

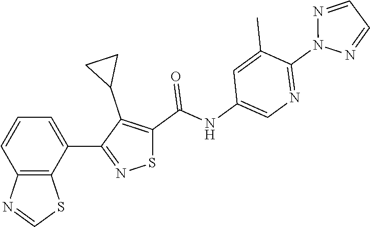 [SMILES: Cc1cc(NC(=O)c2snc(-c3cccc4ncsc34)c2C2CC2)cnc1-n1nccn1]

General procedure D (using 5-methyl-6-(2H-1,2,3-triazol-2-yl)pyridin-3-amine and methyl 3-(benzo[d]thiazol-7-yl)-4-cyclopropylisothiazole-5-carboxylate) yielded the title compound (31 mg, 20%) as a white solid. (ES, m/z): [M+H]⁺ 460; ¹H NMR (400 MHz, DMSO-d₆): δ 11.15 (s, 1H), 9.43 (s, 1H), 8.68 (d, J=2.5 Hz, 1H), 8.27 (d, J=2.5 Hz, 1H), 8.12-8.23 (m, 3H), 8.08 (s, 2H), 7.70 (t, J=7.8 Hz, 1H), 2.23-2.36 (m, 1H), 2.20 (s, 3H), 2.00-2.10 (m, 1H), 0.77-0.92 (m, 2H), 0.20-0.39 (m, 2H).

### EXAMPLE 197—SYNTHESIS OF N-(5-CYANO-6-(2H-1,2,3-TRIAZOL-2-YL) PYRIDIN-3-YL)-4-CYCLOPROPYL-3-(PYRAZOLO[1,5-A]PYRIDIN-4-YL) ISOTHIAZOLE-5-CARBOXAMIDE

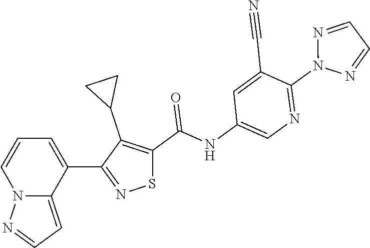 [SMILES: N#Cc1cc(NC(=O)c2snc(-c3cccn4nccc34)c2C2CC2)cnc1-n1nccn1]

General procedure D (using ethyl 4-cyclopropyl-3-(pyrazolo[1,5-a]pyridin-4-yl)isothiazole-5-carboxylate and 5-amino-2-(2H-1,2,3-triazol-2-yl)nicotinonitrile) yielded the title compound (49 mg, 43%) as a white solid. (ES, m/z): [M+H]⁺ 454; ¹H NMR (400 MHz, DMSO-d₆): δ 0.32-0.41 (m, 2H), 0.78-0.89 (m, 2H), 2.11-2.29 (m, 3H), 6.69-6.91 (m, 1H), 7.07 (t, J=7.2 Hz, 1H), 7.69 (d, J=6.4 Hz, 1H). 8.10 (d, J=2.4 Hz, 1H), 8.32 (s, 2H), 8.80-8.90 (m, 2H), 9.10 (d, J=2.4 Hz, 1H), 11.41 (s, 1H).

### EXAMPLE 198—SYNTHESIS OF N-[5-CYANO-6-(1,2,3-TRIAZOL-2-YL)PYRIDIN-3-YL]-4-CYCLOPROPYL-3-(OXAN-3-YL)-1,2-THIAZOLE-5-CARBOXAMIDE

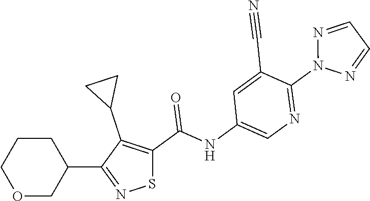 [SMILES: N#Cc1cc(NC(=O)c2snc(C3CCCOC3)c2C2CC2)cnc1-n1nccn1]

General procedure D (using ethyl 4-cyclopropyl-3-(oxan-3-yl)-1,2-thiazole-5-carboxylate and 5-amino-2-(1,2,3-triazol-2-yl)pyridine-3-carbonitrile (26 mg, 21%) as a white solid. (ES, m/z): [M+H]⁺ 422, ¹H NMR (400 MHz, CDCl₃): δ 8.94 (d, J=2.6 Hz, 1H), 8.82 (d, J=2.6 Hz, 1H), 8.64 (s, 1H), 8.03 (s, 2H), 4.00-4.09 (m, 2H), 3.67 (t, J=10.9 Hz, 1H), 3.42-3.50 (m, 2H), 2.06 (s, 1H), 1.95-2.06 (m, 1H), 1.90-1.99 (m, 1H), 1.73-1.90 (m, 1H), 1.79 (s, 1H), 1.23-1.37 (m, 2H), 0.80-0.90 (m, 2H).

### EXAMPLE 199—SYNTHESIS OF N-(5-CYANO-6-(2H-1,2,3-TRIAZOL-2-YL) PYRIDIN-3-YL)-4-CYCLOPROPYL-3-(1,4-DIMETHYL-1H-PYRAZOL-3-YL) ISOTHIAZOLE-5-CARBOXAMIDE

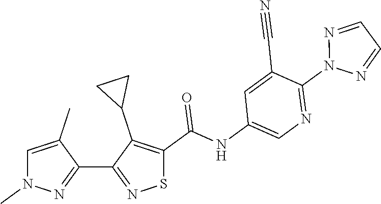 [SMILES: Cc1cn(C)nc1-c1nsc(C(=O)Nc2cnc(-n3nccn3)c(C#N)c2)c1C1CC1]

General procedure H (using ethyl 4-cyclopropyl-3-(1,4-dimethyl-1H-pyrazol-3-yl)isothiazole-5-carboxylate and 5-amino-2-(2H-1,2,3-triazol-2-yl)nicotinonitrile) yielded the title compound (3 mg, 3%) as a white solid. (ES, m/z): [M+H]⁺ 432; ¹H NMR (400 MHz, DMSO-d₆): δ 0.41-0.53 (m, 2H), 0.79-0.89 (m, 2H), 2.16 (s, 3H), 2.36-2.40 (m, 1H), 3.87 (s, 3H), 7.61 (s, 1H), 8.31 (s, 2H), 8.82 (d, J=2.4 Hz, 1H), 9.07 (d, J=2.4 Hz, 1H), 11.38 (br s, 1H).

### EXAMPLE 200—SYNTHESIS OF 3-(BENZO[D]THIAZOL-7-YL)-N-(4-(3-((TERT-BUTYLDIMETHYLSILYL)OXY)AZETIDINE-1-CARBONYL)-3-CHLOROPHENYL)-4-CYCLOPROPYLISOTHIAZOLE-5-CARBOXAMIDE

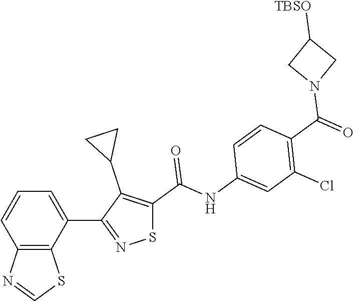 [SMILES: CC(C)(C)[Si](C)(C)OC1CN(C(=O)c2ccc(NC(=O)c3snc(-c4cccc5ncsc45)c3C3CC3)cc2Cl)C1]

General procedure D (using methyl 3-(benzo[d]thiazol-7-yl)-4-cyclopropylisothiazole-5-carboxylate and (4-amino-2-chlorophenyl)(3-((tert-butyldimethylsilyl)oxy)azetidin-1-yl)methanone) yielded the title compound (80 mg, 44%) as a off-white solid. (ES, m/z): [M+H]⁺ 625; ¹H NMR (400 MHz, DMSO-d₆): δ 0.06 (s, 6H), 0.36 (d, J=5.7 Hz, 2H), 0.76-1.02 (m, 4H), 1.23 (s, 2H), 2.20-2.38 (m, 1H), 3.30-3.90 (m, 10H), 4.07 (t, J=8.0 Hz, 1H), 4.24 (m, 1H), 4.51 (q, J=5.8, 5.4 Hz, 1H), 5.85 (d, J=6.1 Hz, 1H), 7.47 (d, J=8.4 Hz, 1H), 7.63-7.72 (m, 1H), 7.76 (t, J=7.8 Hz, 1H), 7.88-8.03 (m, 1H), 8.23 (t, J=7.3 Hz, 2H), 9.49 (s, 1H), 11.04 (s, 1H).

### EXAMPLE 201—SYNTHESIS OF 3-(BENZO[D]THIAZOL-7-YL)-N-(3-CHLORO-4-(3-HYDROXYAZETIDINE-1-CARBONYL)PHENYL)-4-CYCLOPROPYL-ISOTHIAZOLE-5-CARBOXAMIDE

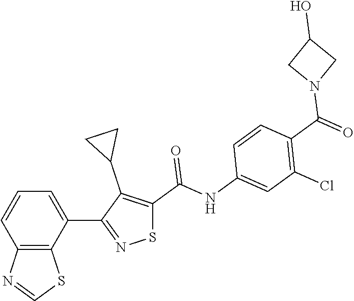 [SMILES: O=C(Nc1ccc(C(=O)N2CC(O)C2)c(Cl)c1)c1snc(-c2cccc3ncsc23)c1C1CC1]

General procedure E (using 3-(benzo[d]thiazol-7-yl)-N-(4-(3-((tert-butyldimethylsilyl)oxy) azetidine-1-carbonyl)-3-chlorophenyl)-4-cyclopropylisothiazole-5-carboxamide and CsF) yielded the title compound (32 mg, 32%) as a off-white solid. (ES, m/z): [M+H]⁺ 511; ¹H NMR (400 MHz, DMSO-d₆): δ 0.30-0.40 (m, 2H), 0.85-0.95 (m, 2H), 2.20-2.40 (m, 1H), 3.65-3.80 (m, 2H), 4.05-4.20 (m, 1H), 4.17-4.35 (m, 1H), 4.52 (q, J=5.9 Hz, 1H), 5.85 (d, J=6.1 Hz, 1H), 7.47 (d, J=8.4 Hz, 1H), 7.69 (d, J=8.4 Hz. 1H), 7.76 (t, J=7.6 Hz, 1H), 7.96 (s, 1H), 8.23 (t, J=7.6 Hz, 2H), 9.49 (s, 1H), 11.04 (s, 1H).

### EXAMPLE 202—SYNTHESIS OF N-(5-CHLORO-6-(2H-1,2,3-TRIAZOL-2-YL) PYRIDIN-3-YL)-4-CYCLOPROPYL-3-(QUINOLIN-5-YL)ISOTHIAZOLE-5-CARBOXAMIDE

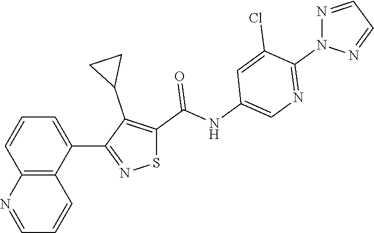 [SMILES: O=C(Nc1cnc(-n2nccn2)c(Cl)c1)c1snc(-c2cccc3ncccc23)c1C1CC1]

General procedure D (using methyl 4-cyclopropyl-3-(quinolin-5-yl)-1,2-thiazole-5-carboxylate and 2-(trifluoromethyl)pyridin-4-amine) yielded the title compound (32 mg, 29%) as a yellow solid. (ES, m/z): [M+H]⁺ 474 ¹H NMR (400 MHz, DMSO-d₆): δ 11.37 (s, 1H), 9.00 (dd, J=4.2, 1.6 Hz, 1H), 8.87 (d, J=2.4 Hz, 1H), 8.68 (d, J=2.4 Hz, 1H), 8.20 (d, J=4.2 Hz, 3H), 8.16 (dd, J=8.8, 1.6 Hz, 1H), 7.93 (dd, J=8.8, 7.2 Hz, 1H), 7.75-7.81 (m, 1H), 7.60 (dd, J=8.6, 4.2 Hz, 1H), 1.95-2.05 (m, 1H), 0.51-0.62 (m, 2H), 0.19-0.32 (m, 2H).

### EXAMPLE 203—SYNTHESIS OF 4-CYCLOPROPYL-3-(QUINOLIN-5-YL)-N-(2-(TRIFLUOROMETHYL)PYRIDIN-4-YL)ISOTHIAZOLE-5-CARBOXAMIDE

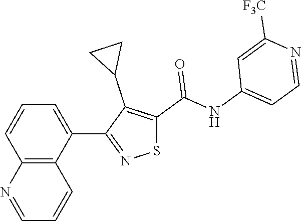 [SMILES: O=C(Nc1ccnc(C(F)(F)F)c1)c1snc(-c2cccc3ncccc23)c1C1CC1]

General procedure D (using methyl 4-cyclopropyl-3-(quinolin-5-yl)-1,2-thiazole-5-carboxylate and 2-(trifluoromethyl)pyridin-4-amine) yielded the title compound (22 mg, 30%) as a white solid. (ES, m/z): [M+H]⁺ 441.1 ¹H NMR (400 MHz, DMSO-d₆): δ 11.40 (s, 1H), 8.99 (dd, J=4.2, 1.6 Hz, 1H), 8.74 (d, J=5.6 Hz, 1H), 8.24 (d, J=2.0 Hz, 1H), 8.19 (d, J=8.4 Hz, 1H), 8.11-8.17 (m, 1H), 7.96 (dd, J=5.6, 2.0 Hz, 1H), 7.91 (dd. J=8.4, 7.2 Hz, 1H), 7.76 (dd, J=7.2, 1.2 Hz, 1H), 7.59 (dd, J=8.4, 4.2 Hz, 1H), 1.90-2.00 (m, 1H), 0.45-0.58 (m, 2H), 0.18-0.27 (m, 2H).

### EXAMPLE 204—SYNTHESIS OF N-(5-CYANO-6-(2H-1,2,3-TRIAZOL-2-YL) PYRIDIN-3-YL)-4-CYCLOPROPYL-3-(1-METHYL-1H-BENZO[D][1,2,3]TRIAZOL-7-YL) ISOTHIAZOLE-5-CARBOXAMIDE

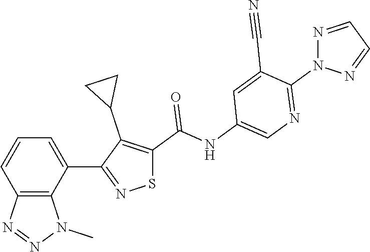 [SMILES: Cn1nnc2cccc(-c3nsc(C(=O)Nc4cnc(-n5nccn5)c(C#N)c4)c3C3CC3)c21]

General procedure D (ethyl 4-cyclopropyl-3-(1-methyl-1H-benzo[d][1,2,3]triazol-7-yl)isothiazole-5-carboxylate and 5-amino-2-(2H-1,2,3-triazol-2-yl)nicotinonitrile) yielded the title compound (24 mg, 13%) as a white solid. (ES, m/z): [M+H]⁺ 469; ¹H NMR (400 MHz, DMSO-d₆): δ 0.29-0.52 (m, 2H), 0.61-0.78 (m, 2H), 2.01-2.10 (m, 1H), 3.95 (s, 3H), 7.39-7.65 (m, 1H), 7.72 (d, J=6.8 Hz, 1H), 8.23 (d, J=8.4 Hz, 1H), 8.33 (s, 2H), 8.87 (d, J=2.4 Hz, 1H), 9.09 (s, 1H), 11.42 (s, 1H).

### EXAMPLE 205—SYNTHESIS OF 3-(1-ACETYLPIPERIDIN-4-YL)-N-(5-CYANO-6-(2H-1,2,3-TRIAZOL-2-YL)PYRIDIN-3-YL)-4-CYCLOPROPYLISOTHIAZOLE-5-CARBOXAMIDE

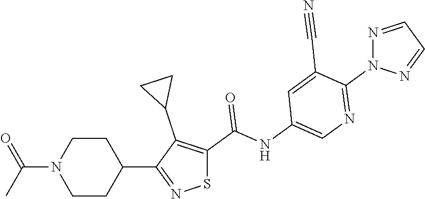 [SMILES: CC(=O)N1CCC(c2nsc(C(=O)Nc3cnc(-n4nccn4)c(C#N)c3)c2C2CC2)CC1]

A solution of N-(5-cyano-6-(2H-1,2,3-triazol-2-yl)pyridin-3-yl)-4-cyclopropyl-3-(piperidin-4-yl)isothiazole-5-carboxamide (25 mg, 0.06 mmol, 1.0 eq.) and DIEA (23 mg, 0.18 mmol, 3.0 eq.) and AcOH (11 mg, 0.18 mmol, 3.1 eq.), HATU (27 mg, 0.07 mmol, 1.2 eq.) in DMF (1 mL) was stirred for 2 h at room temperature under nitrogen. The resulting mixture was diluted with water (10 mL), and extracted with EtOAc (3×20 mL). The combined organic layers were washed with brine (5 mL), dried over anhydrous Na₂SO₄, and concentrated under reduced pressure. The crude product was purified by Prep-HPLC with the following conditions (Column: XSelect CSH Fluoro Phenyl, 30*150 mm, 5 m; Mobile Phase A: Water (0.1% FA), Mobile Phase B: ACN; Flow rate: 60 mL/min; Gradient: 30% B to 41% B in 7 min, 41% B; Wave Length: 254; 220 nm; RT1(min): 6.5; Number Of Runs: 0) to afford the title compound (8 mg, 30%) as an off-white solid. (ES, m/z): [M+H]⁺ 463; ¹H NMR (400 MHz, DMSO-d₆): 0.55-0.66 (m, 2H), 0.93-1.06 (m, 2H), 1.48-1.59 (m, 1H), 1.67-1.83 (m, 1H), 1.85-2.12 (m, 6H), 2.65-2.78 (m, TH), 3.19 (d, J=11.6 Hz, 1H), 3.94 (d, J=13.6 Hz, TH), 4.49 (d, J=13.2 Hz, TH), 8.31 (s, 2H), 8.80 (d, J=2.4 Hz, 1H), 9.05 (d, J=2.4 Hz, 1H), 11.30 (br s, 1H).

### EXAMPLE 206—SYNTHESIS OF N-(5-CYANO-6-(2H-1,2,3-TRIAZOL-2-YL) PYRIDIN-3-YL)-4-CYCLOPROPYL-3-(1-METHYLPIPERIDIN-4-YL)ISOTHIAZOLE-5-CARBOXAMIDE

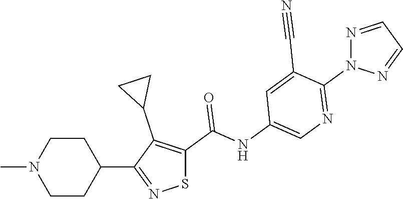 [SMILES: CN1CCC(c2nsc(C(=O)Nc3cnc(-n4nccn4)c(C#N)c3)c2C2CC2)CC1]

A solution of N-(5-cyano-6-(2H-1,2,3-triazol-2-yl)pyridin-3-yl)-4-cyclopropyl-3-(piperidin-4-yl)isothiazole-5-carboxamide (80 mg, 0.2 mmol, 1 eq.) and HCHO (18 mg, 0.6 mmol, 3.2 eq.) and NaBH(OAc)₃ (121 mg, 0.6 mmol, 3.0 eq.) in MeOH (5 mL) was stirred overnight at room temperature under nitrogen. The resulting mixture was diluted with water (10 mL), and extracted with EtOAc (3×50 mL). The combined organic layers were washed with brine (10 mL), dried over anhydrous Na₂SO₄, and concentrated under reduced pressure. The crude product was purified by Prep-HPLC with the following conditions (Column: XBridge Shield RP18 OBD Column, 30*150 mm, 5 m; Mobile Phase A: Water (10 mmol/L NH₄HCO₃+0.1% NH₃·H₂O), Mobile Phase B: ACN; Flow rate: 60 mL/min; Gradient: 30% B to 55% B in 8 min, 55% B; Wave Length: 220/254 nm; RT1(min): 7.63; Injection Volume: 1.5 mL; Number Of Runs: 2) to afford the title compound (8 mg, 9%) as an off-white solid. (ES, m/z): [M+H]⁺ 435; ¹H NMR (400 MHz, DMSO-d₆): 0.53-0.64 (m, 2H), 0.92-1.03 (m, 2H), 1.76-1.91 (m, 4H), 1.92-2.01 (m, 1H), 2.07 (s, 2H), 2.24 (s, 3H), 2.93 (d, J=11.2 Hz, 2H), 3.07 (d, J=10.0 Hz, 1H), 8.20 (d, J=1.6 Hz, 1H), 8.31 (s, 2H), 8.80 (d, J=2.4 Hz, 1H), 9.05 (d, J=2.4 Hz, 1H), 11.40 (br s, 1H).

### EXAMPLE 207—SYNTHESIS OF N-(5-CYANO-6-(2H-1,2,3-TRIAZOL-2-YL)PYRIDIN-3-YL)-4-CYCLOPROPYL-3-(QUINOLIN-5-YL)ISOTHIAZOLE-5-CARBOXAMIDE

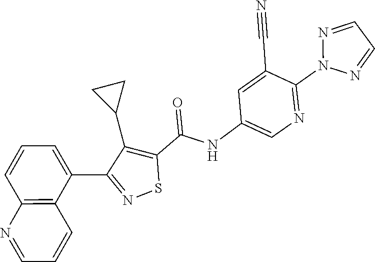 [SMILES: N#Cc1cc(NC(=O)c2snc(-c3cccc4ncccc34)c2C2CC2)cnc1-n1nccn1]

General procedure D (using methyl 4-cyclopropyl-3-(quinolin-5-yl)isothiazole-5-carboxylate and 5-amino-2-(2H-1,2,3-triazol-2-yl)nicotinonitrile) yielded the title compound (2 mg, 6%) as a white solid. (ES, m/z): [M+H]⁺ 465; ¹H NMR (400 MHz, DMSO-d₆): δ 8.96-9.04 (m, 2H), 8.89 (d, J=2.4 Hz, 1H), 8.30 (s, 2H), 8.19 (d, J=8.4 Hz, 1H), 8.12 (d, J=8.4 Hz, 1H), 7.87-7.96 (m, 1H), 7.72-7.79 (m, 1H), 7.55-7.63 (m, 1H), 1.96-2.10 (m, 1H), 0.51-0.60 (m, 2H), 0.23-0.32 (m, 2H).

### EXAMPLE 208—SYNTHESIS OF 4-CYCLOPROPYL-3-ISOPROPYL-N-(2-(TRIFLUOROMETHYL)PYRIDIN-4-YL)ISOTHIAZOLE-5-CARBOXAMIDE

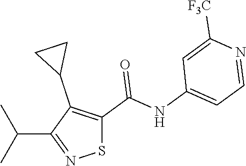 [SMILES: CC(C)c1nsc(C(=O)Nc2ccnc(C(F)(F)F)c2)c1C1CC1]

General procedure A (using 4-cyclopropyl-3-isopropylisothiazole-5-carboxylic acid and 2-(trifluoromethyl)pyridin-4-amine) yielded the title compound (56 mg, 29%) as a white solid. (ES, m/z): [M+H]⁺ 356; ¹H NMR (400 MHz, DMSO-d₆): δ 0.48-0.56 (m, 2H), 0.88-0.98 (m, 2H), 1.89-1.99 (m, 1H), 3.45 (p, J=6.8 Hz, 1H), 7.90 (dd, J=5.6, 2.0 Hz, 1H), 8.18 (d, J=2.0 Hz, 1H), 8.70 (d, J=5.6 Hz, 1H), 11.08 (s, 1H).

### EXAMPLE 209—SYNTHESIS OF 4-CYCLOPROPYL-3-(DIMETHYLAMINO)-N-[2-(TRIFLUOROMETHYL)PYRIDIN-4-YL]-1,2-THIAZOLE-5-CARBOXAMIDE

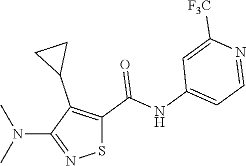 [SMILES: CN(C)c1nsc(C(=O)Nc2ccnc(C(F)(F)F)c2)c1C1CC1]

A solution of 4-cyclopropyl-5-((2-(trifluoromethyl)pyridin-4-yl)carbamoyl)isothiazol-3-yl trifluoromethanesulfonate (195 mg, 0.4 mmol, 1.0 eq.) in THF (4 mL) was treated with NEt₃ (128.3 mg, 1.3 mmol, 3.0 eq.) for 30 min at 0° C. under nitrogen followed by the addition of dimethylamine (2 Min THF, 8 mL, 121 mmol, 285 eq.) dropwise at 0° C. The resulting mixture was stirred for 3 h at 50° C. under nitrogen, and then concentrated under reduced pressure. The crude product (40 mg) was purified by Prep-HPLC with the following conditions (Column: Xselect CSH C18 OBD Column 30*150 mm 5 m, n; Mobile Phase A: Water (0.1% FA), Mobile Phase B: ACN; Flow rate: 60 mL/min; Gradient: 48% B to 61% B in 7 min, 61% B; Wave Length: 254; 220 nm; RT1(min): 6.32; Injection Volume: 2000 mL; Number Of Runs: 1) to afford the title compound (19 mg, 12%) as a white solid. (ES, m/z): [M+H]⁺ 357; ¹H NMR (400 MHz, DMSO-d₆): δ 0.58-0.44 (m, 2H), 0.97-0.83 (m, 2H), 1.97-2.04 (m, 1H), 3.02 (s, 6H), 7.93 (dd, J=5.6, 2.0 Hz, 1H), 8.18 (d, J=1.6 Hz, 1H), 8.70 (d, J=5.6 Hz, 1H), 11.14 (s, 1H).

### EXAMPLE 210—SYNTHESIS OF N-(5-CYANO-6-(2H-1,2,3-TRIAZOL-2-YL) PYRIDIN-3-YL)-4-CYCLOPROPYL-3-(2-METHYL-2H-BENZO[D][1,2,3]TRIAZOL-4-YL) ISOTHIAZOLE-5-CARBOXAMIDE

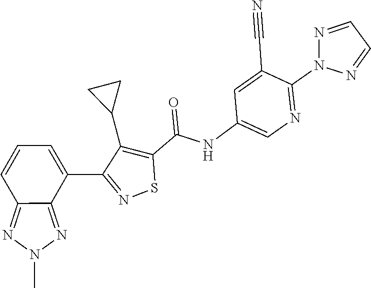 [SMILES: Cn1nc2cccc(-c3nsc(C(=O)Nc4cnc(-n5nccn5)c(C#N)c4)c3C3CC3)c2n1]

General procedure D (using ethyl 4-cyclopropyl-3-(2-methyl-2H-indazol-7-yl)isothiazole-5-carboxylate and 5-amino-2-(2H-1,2,3-triazol-2-yl)nicotinonitrile) yielded the title compound (38 mg, 30%) as a white solid. (ES, m/z): [M+H]⁺ 469; ¹H NMR (400 MHz, DMSO-d₆): δ 0.19-0.31 (m, 2H), 0.40-0.70 (m, 2H), 2.11-2.29 (m, 1H), 7.47-7.77 (m, 2H), 8.08 (dd, J=8.4, 0.8 Hz, 1H), 8.33 (s, 1H), 8.87 (d, J=2.4 Hz, 1H), 9.12 (d, J=2.4 Hz, 1H), 11.45 (s, 1H).

### EXAMPLE 211—SYNTHESIS OF N-(5-CYANO-6-(2H-1,2,3-TRIAZOL-2-YL) PYRIDIN-3-YL)-4-CYCLOPROPYL-3-(1-METHYL-1H-BENZO[D][1,2,3]TRIAZOL-4-YL)ISOTHIAZOLE-5-CARBOXAMIDE

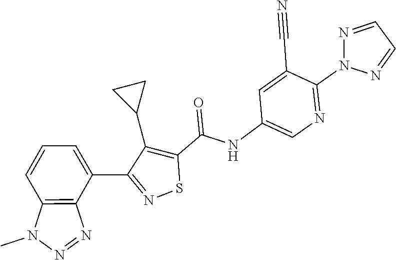 [SMILES: Cn1nnc2c(-c3nsc(C(=O)Nc4cnc(-n5nccn5)c(C#N)c4)c3C3CC3)cccc21]

General procedure D (using ethyl 4-cyclopropyl-3-(1-methyl-1H-benzo[d][1,2,3]triazol-4-yl)isothiazole-5-carboxylate and 5-amino-2-(2H-1,2,3-triazol-2-yl)nicotinonitrile) yielded the title compound (36 mg, 31%) as a white solid. (ES, m/z): [M+H]⁺ 469; ¹H NMR (400 MHz, DMSO-d₆): δ 0.10-0.38 (m, 2H), 0.40-0.67 (m, 2H), 2.29-2.42 (m, 1H), 4.39 (s, 3H), 7.47-7.63 (m, 1H), 7.64-7.85 (m, 1H), 7.91-8.19 (m, 1H), 8.32 (s, 2H), 8.88 (d, J=2.4 Hz, 1H), 9.14 (d, J=2.4 Hz, 1H), 11.44 (s, 1H).

### EXAMPLE 212—SYNTHESIS OF 4-CYCLOPROPYL-3-PHENYL-N-(2-(TRIFLUORO-METHYL)PYRIDIN-4-YL)ISOTHIAZOLE-5-CARBOXAMIDE

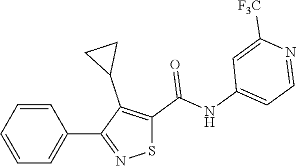 [SMILES: O=C(Nc1ccnc(C(F)(F)F)c1)c1snc(-c2ccccc2)c1C1CC1]

General procedure A (using 4-cyclopropyl-3-phenylisothiazole-5-carboxylic acid and 2-(trifluoromethyl)pyridin-4-amine) yielded the title compound (57 mg, 24%) as a white solid. (ES, m/z): [M+H]⁺ 390.1, ¹H NMR (400 MHz, DMSO-d₆): δ 0.30-0.40 (m, 2H), 0.85-0.92 (m, 2H), 2.20-2.35 (m, 1H), 7.45-7.55 (m, 3H), 7.80-7.90 (m, 2H), 7.94 (dd, J=5.2, 1.2 Hz, 1H), 8.22 (d, J=1.6 Hz, 1H), 8.73 (d, J=5.2 Hz, 1H), 11.37 (s, 1H).

### EXAMPLE 213—SYNTHESIS OF 3-(BENZYLOXY)-N-(5-CYANO-6-(2H-1,2,3-TRIAZOL-2-YL)PYRIDIN-3-YL)-4-CYCLOPROPYLISOTHIAZOLE-5-CARBOXAMIDE

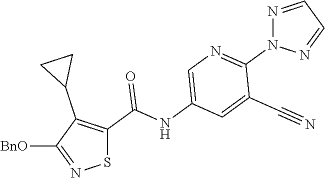 [SMILES: N#Cc1cc(NC(=O)c2snc(OCc3ccccc3)c2C2CC2)cnc1-n1nccn1]

General procedure D (using ethyl 3-(benzyloxy)-4-cyclopropylisothiazole-5-carboxylate and N-(5-amino-2-(2H-1,2,3-triazol-2-yl)nicotinonitrile) yielded the title compound (500 mg, 68%) as a white solid. (ES, m/z): [M+H]⁺ 444.0.

### EXAMPLE 214—SYNTHESIS OF N-(6-(3-((TERT-BUTYLDIMETHYLSILYL)OXY) AZETIDINE-1-CARBONYL)-5-CHLOROPYRIDIN-3-YL)-4-CYCLOPROPYL-3-PHENYLISOTHIAZOLE-5-CARBOXAMIDE

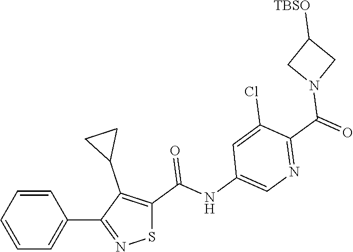 [SMILES: CC(C)(C)[Si](C)(C)OC1CN(C(=O)c2ncc(NC(=O)c3snc(-c4ccccc4)c3C3CC3)cc2Cl)C1]

General procedure A (using 3-chloro-5-(4-cyclopropyl-3-phenylisothiazole-5-carboxamido) picolinic acid and 3-((tert-butyldimethylsilyl)oxy)azetidine) yielded the title compound (100 mg, 88%) as a white solid. (ES, m/z): [M+H]⁺ 569.

### EXAMPLE 215—SYNTHESIS OF N-(6-(3-((TERT-BUTYLDIMETHYLSILYL)OXY) AZETIDINE-1-CARBONYL)PYRIDIN-3-YL)-4-CYCLOPROPYL-3-PHENYL-ISOTHIAZOLE-5-CARBOXAMIDE

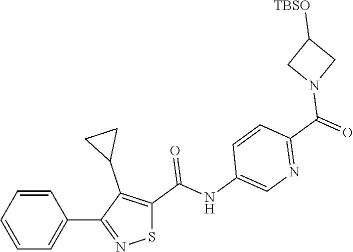 [SMILES: CC(C)(C)[Si](C)(C)OC1CN(C(=O)c2ccc(NC(=O)c3snc(-c4ccccc4)c3C3CC3)cn2)C1]

General procedure A (using 5-(4-cyclopropyl-3-phenylisothiazole-5-carboxamido)picolinic acid and 3-((tert-butyldimethylsilyl)oxy)azetidine) yielded the title compound (80 mg, 55%) as a white solid. (ES, m/z): [M+H]⁺ 535.2; ¹H NMR (400 MHz, CDCl₃): δ 0.08 (s, 6H), 0.50-0.60 (m, 2H), 0.90 (s, 9H), 1.05-1.15 (m, 2H), 2.20-2.30 (m, 1H), 4.01-4.10 (m, 1H), 4.40-4.50 (m, 2H), 4.63-4.75 (m, 1H), 4.85-4.95 (m, 1H), 7.46-7.48 (m, 2H), 7.73-7.74 (m, 2H), 8.12 (d, J=8.8 Hz, 1H), 8.53 (d, J=8.8 Hz, 1H), 9.06 (s, 1H), 9.52 (s, 1H).

### EXAMPLE 216—SYNTHESIS OF N-(5-CYANO-6-(2H-1,2,3-TRIAZOL-2-YL) PYRIDIN-3-YL)-3-(1-METHYL-2-OXO-1,2-DIHYDROPYRIDIN-4-YL)-4-(TRIFLUORO-METHYL)ISOTHIAZOLE-5-CARBOXAMIDE (I-128)

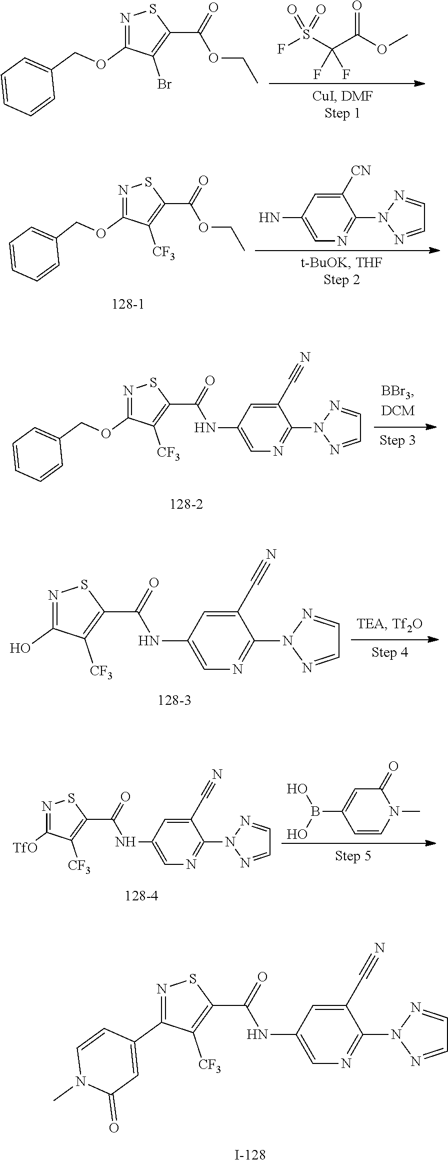

Step 1: Synthesis of 128-1: A solution of ethyl 3-(trifluoromethanesulfonyloxy)-4-(trifluoromethyl)-1,2-thiazole-5-carboxylate (2 g, 5.358 mmol, 1 equiv) in DMF (10 mL) was treated with 1-methyl-2-oxopyridin-3-ylboronic acid (0.98 g, 6.430 mmol, 1.2 equiv) for 10 min at room temperature under a nitrogen atmosphere followed by the addition of CuI (2.04 g, 11.076 mmol, 2 equiv) in portions at room temperature. The resulting mixture was stirred for an additional 2 h at room temperature. The resulting mixture was filtered, and the filter cake was washed with EtOAc (2×20 mL). The filtrate was concentrated under reduced pressure. The residue was purified by reversed-phase flash chromatography with the following conditions: column: C18 silica gel; mobile phase: MeCN in water (0.1% TFA); 20% to 50% gradient in 20 min; detector: UV 254 nm. This resulted in 128-1 (1.4 g, 78.62%) as a brown solid.

Step 2: Synthesis of 128-2: A solution of 128-1 (1 g, 3.018 mmol, 1 equiv) in THF (10 mL) was treated with 5-amino-2-(1,2,3-triazol-2-yl)pyridine-3-carbonitrile (0.67 g, 3.622 mmol, 1.2 equiv) for 30 min at room temperature under a nitrogen atmosphere followed by the addition of t-BuOK (0.68 g, 6.036 mmol, 2 equiv) in portions at room temperature. The resulting mixture was stirred for an additional 2 h at room temperature. The resulting mixture was concentrated under reduced pressure. The residue was purified by reversed-phase flash chromatography with the following conditions: column: C18 silica gel; mobile phase: MeCN in water (0.1% TFA); 20% to 50% gradient in 20 min; detector: UV 254 nm. This resulted in 128-2 (250 mg, 17.57%) as a white solid.

Step 3: Synthesis of 128-3: To a stirred solution of 128-2 (250 mg, 0.530 mmol, 1 equiv) in DCM (5 mL) was added BBr₃ (398.6 mg, 1.590 mmol, 3 equiv) dropwise at 0° C. under a nitrogen atmosphere. The resulting mixture was stirred for an additional 2 h at room temperature. The resulting mixture was quenched with water and then concentrated under reduced pressure. The residue was purified by reversed-phase flash chromatography with the following conditions: column: C18 silica gel; mobile phase: MeCN in water (0.1% TFA); 30% to 70% gradient in 20 min; detector: UV 254 nm. This resulted in 128-3 (90 mg, 44.51%) as a white solid.

Step 4: Synthesis of 128-4: A solution of 128-3 (90 mg, 0.236 mmol, 1 equiv) in DCM (2 mL) was treated with TEA (71.7 mg, 0.708 mmol, 3 equiv) for 15 min at room temperature under a nitrogen atmosphere followed by the addition of Tf₂O (99.9 mg, 0.354 mmol, 1.5 equiv) dropwise at 0° C. The resulting mixture was stirred for an additional 2 h at room temperature. The resulting mixture was concentrated under reduced pressure. The residue was purified by reversed-phase flash chromatography with the following conditions: column: C18 silica gel; mobile phase: MeCN in water (0.1% TFA); 20% to 50% gradient in 20 min; detector: UV 254 nm. This resulted in 128-4 (40 mg, 33.01%) as a white solid.

Step 5: Synthesis of N-(5-cyano-6-(2H-1,2,3-triazol-2-yl)pyridin-3-yl)-3-(1-methyl-2-oxo-1,2-dihydropyridin-4-yl)-4-(trifluoromethyl)isothiazole-5-carboxamide (I-128): A solution of 128-4 (5 mg, 0.010 mmol, 1 equiv) in toluene (1 mL) was treated with 1-methyl-2-oxopyridin-4-ylboronic acid (1.49 mg, 0.010 mmol, 1 equiv) for 10 min at room temperature under a nitrogen atmosphere followed by the addition of XPhos (1.39 mg, 0.003 mmol, 0.3 equiv), XPhos Pd G3 (3.71 mg, 0.008 mmol, 0.8 equiv) and K₃PO₄ (2.27 mg, 0.011 mmol, 1.1 equiv) in portions at room temperature. The resulting mixture was stirred for an additional 2 h at 60° C. The residue was purified by reversed-phase flash chromatography with the following conditions: column: C18 silica gel; mobile phase: MeCN in water (0.1% TFA); 30% to 80% gradient in 25 min; detector: UV 254 nm. This result in N-(5-cyano-6-(2H-1,2,3-triazol-2-yl)pyridin-3-yl)-3-(1-methyl-2-oxo-1,2-dihydropyridin-4-yl)-4-(trifluoromethyl)isothiazole-5-carboxamide (1-128, 1 mg, 21.73%) as a white solid. LCMS (ES, m/z): [M+1]⁺=473; ¹H NMR (400 MHz, DMSO-d₆) δ 3.56 (s, 3H), 6.35-6.37 (d, J=7.2,1H), 6.51 (m, 1H), 7.35-7.31 (m, 1H), 7.84-7.86 (d, J=7.2 Hz, 1H), 8.29 (s, 2H), 8.78-8.79 (d, J=2.4 Hz, 1H), 8.90 (m, 1H).

### EXAMPLE 217—SYNTHESIS OF 3-(1-ACETYLAZETIDIN-3-YL)-N-(5-CYANO-6-(2H-1,2,3-TRIAZOL-2-YL)PYRIDIN-3-YL)-4-(TRIFLUOROMETHYL) ISOTHIAZOLE-5-CARBOXAMIDE (I-129)

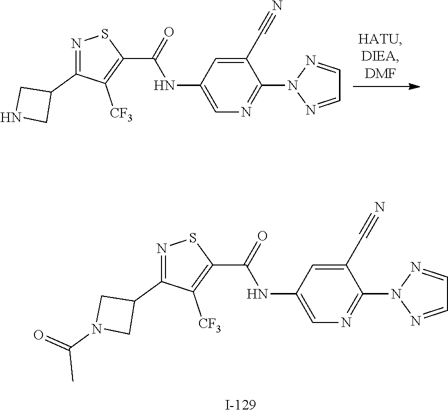 [SMILES: N#Cc1cc(NC(=O)c2snc(C3CNC3)c2C(F)(F)F)cnc1-n1nccn1 ; CC(=O)N1CC(c2nsc(C(=O)Nc3cnc(-n4nccn4)c(C#N)c3)c2C(F)(F)F)C1]

To a stirred solution of 3-(azetidin-3-yl)-N-[5-cyano-6-(1,2,3-triazol-2-yl) pyridin-3-yl]-4-(trifluoromethyl)-1,2-thiazole-5-carboxamide (120 mg, 0.285 mmol, 1 equiv) and HATU (141.11 mg, 0.370 mmol, 1.3 equiv) in DMF (3 mL) was added DIEA (73.79 mg, 0.570 mmol, 2 equiv) and AcOH (25.71 mg, 0.427 mmol, 1.5 equiv) in portions at room temperature under a nitrogen atmosphere. The resulting mixture was stirred for 18 h at room temperature under a nitrogen atmosphere. The reaction was quenched with water at room temperature. The resulting mixture was extracted with EtOAc (2×50 mL). The combined organic layers were washed with brine (1×30 mL) and dried over anhydrous Na₂SO₄. After filtration, the filtrate was concentrated under reduced pressure. The crude product (100 mg) was purified by prep-HPLC with the following conditions: (column: XBridge Prep OBD C18 column: 30*150 mm, 5 μm; mobile phase A: water (10 mmol/L NH₄HCO₃), mobile phase B: ACN; flow rate: 60 mL/min; gradient: 18% B to 40% B in 8 min, 40% B; wavelength: 254 nm; RT1(min): 7.97) to afford 3-(1-acetylazetidin-3-yl)-N-[5-cyano-6-(1,2,3-triazol-2-yl)pyridin-3-yl]-4-(trifluoromethyl)-1,2-thiazole-5-carboxamide (I-129, 8.3 mg, 5.67%) as a white solid. LCMS (ES, m/z): [M+H]⁺=463; ¹H NMR (400 MHz, DMSO-d₆): δ 1.80 (s, 3H), 4.05-4.29 (m, 3H), 4.34-4.44 (m, 1H), 4.52 (d, J=7.6 Hz, 1H), 8.31 (s, 2H), 8.75 (d, J=2.6 Hz, 1H), 8.94 (s, 1H), 11.81 (s, 1H).

### EXAMPLE 218—SYNTHESIS OF N-(5-CYANO-6-(2H-1,2,3-TRIAZOL-2-YL) PYRIDIN-3-YL)-3-(1-METHYLAZETIDIN-3-YL)-4-(TRIFLUOROMETHYL) ISOTHIAZOLE-5-CARBOXAMIDE (I-130)

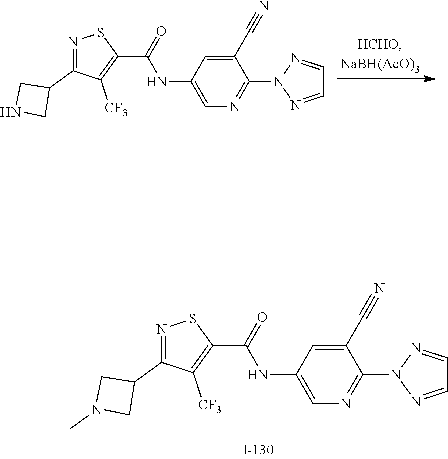 [SMILES: N#Cc1cc(NC(=O)c2snc(C3CNC3)c2C(F)(F)F)cnc1-n1nccn1 ; CN1CC(c2nsc(C(=O)Nc3cnc(-n4nccn4)c(C#N)c3)c2C(F)(F)F)C1]

To a stirred solution of 3-(azetidin-3-yl)-N-[5-cyano-6-(1,2,3-triazol-2-yl)pyridin-3-yl]-4-(trifluoromethyl)-1,2-thiazole-5-carboxamide (130 mg, 0.309 mmol, 1 equiv) and HCHO (13.93 mg, 0.464 mmol, 1.5 equiv) in MeOH (1 mL) was added NaBH(AcO)₃ (131.09 mg, 0.618 mmol, 2 equiv) in portions at room temperature under a nitrogen atmosphere. The resulting mixture was stirred for 5 h at room temperature under a nitrogen atmosphere. The reaction was quenched with water at room temperature. The resulting mixture was extracted with EtOAc (2×50 mL). The combined organic layers were washed with brine (1×30 mL) and dried over anhydrous Na₂SO₄. After filtration, the filtrate was concentrated under reduced pressure. The crude product (100 mg) was purified by prep-HPLC with the following conditions: (column: XBridge Prep OBD C18 column: 30*150 mm, 5 μm; mobile phase A: water (10 mmol/L NH₄HCO₃), mobile phase B: ACN; flow rate: 60 mL/min; gradient: 18% B to 40% B in 8 min, 40% B; wavelength: 254 nm; RT1(min): 7.97) to afford N-[5-cyano-6-(1,2,3-triazol-2-yl)pyridin-3-yl]-3-(1-methylazetidin-3-yl)-4-(trifluoromethyl)-1,2-thiazole-5-carboxamide (I-130, 16.6 mg, 11.68%) as a yellow solid. LCMS (ES, m/z): [M+H]⁺=435; ¹H NMR (400 MHz, DMSO-d₆): δ 2.31 (s, 3H), 3.44 (s, 2H), 3.72 (d, J=7.8 Hz, 2H), 3.83-4.04 (m, 1H), 8.30 (m, 2H), 8.76 (d, J=2.6 Hz, 1H), 8.90 (s, 1H).

### EXAMPLE 219—SYNTHESIS OF N-(5-CYANO-6-(2H-1,2,3-TRIAZOL-2-YL) PYRIDIN-3-YL)-4-CYCLOPROPYL-3-(IMIDAZO[1,2-A]PYRIDIN-5-YL) ISOTHIAZOLE-5-CARBOXAMIDE (I-131)

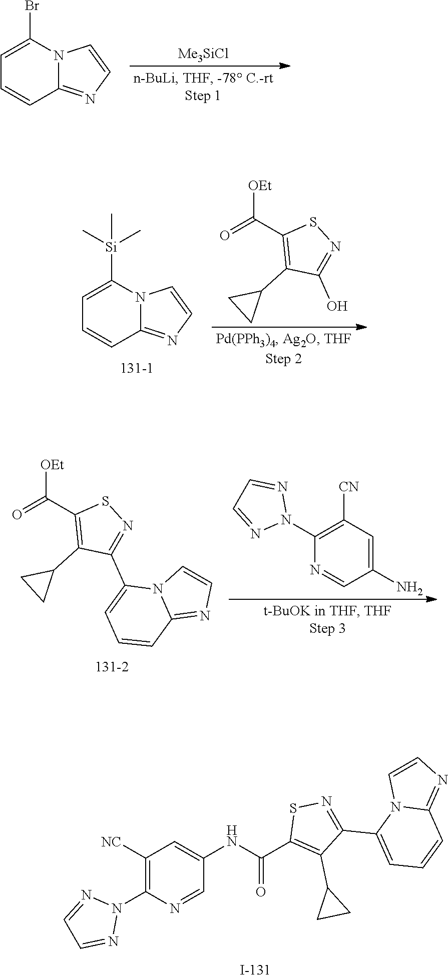

Step 1: Synthesis of 131-1: A solution of 5-bromoimidazo[1,2-a]pyridine (1 g, 5.075 mmol, 1 equiv) in THF (20 mL) was treated with n-BuLi (0.98 g, 15.225 mmol, 3 equiv) for 30 min at −78° C. under a nitrogen atmosphere followed by the addition of chlorotrimethylsilane (1.10 g, 10.150 mmol, 2 equiv) in portions at −78° C. The resulting mixture was stirred for 2 h at −78° C. under a nitrogen atmosphere. The resulting mixture was diluted with water (20 mL). The resulting mixture was extracted with EtOAc (3×20 mL). The combined organic layers were washed with brine (1×50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 60% to 80% gradient in 30 min; detector: UV 254 nm. This resulted in 5-(trimethylsilyl)imidazo[1,2-a]pyridine (131-1, 500 mg, 51.76%) as alight yellow oil. LCMS (ES, m/z): [M+H]⁺⁼191.

Step 2: Synthesis of 131-2: A solution of 5-(trimethylsilyl)imidazo[1,2-a]pyridine (360 mg, 1.892 mmol, 1 equiv) and ethyl 4-cyclopropyl-3-(trifluoromethanesulfonyloxy)-1,2-thiazole-5-carboxylate (320 mg. 0.927 mmol, 0.49 equiv) and Ag₂O (400 mg, 1.726 mmol, 0.91 equiv), Pd(PPh₃)₄(300 mg, 0.260 mmol, 0.14 equiv) in THF (8 mL) was stirred for 36 h at 60° C. under a nitrogen atmosphere. The resulting mixture was diluted with water (20 mL). The resulting mixture was extracted with EtOAc (3×20 mL). The combined organic layers were washed with brine (1×50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 50% to 75% gradient in 30 min; detector: UV 254 nm. This resulted in ethyl 4-cyclopropyl-3-{imidazo[1,2-a]pyridin-5-yl}-1,2-thiazole-5-carboxylate (131-2, 120 mg, 20.24%) as an off-white solid. LCMS (ES, m/z): [M+H]⁺=314.

Step 3: Synthesis of N-(5-cyano-6-(2H-1,2,3-triazol-2-yl)pyridin-3-yl)-4-cyclopropyl-3-(imidazo[1,2-a]pyridin-5-yl)isothiazole-5-carboxamide (1-131) A mixture of ethyl 4-cyclopropyl-3-{imidazo[1,2-a]pyridin-5-yl}-1,2-thiazole-5-carboxylate (50 mg, 0.160 mmol, 1 equiv), 5-amino-2-(1,2,3-triazol-2-yl)pyridine-3-carbonitrile (36 mg, 0.193 mmol, 1.21 equiv), and t-BuOK (54 mg, 0.481 mmol, 3.02 equiv) in THF (2 mL) was stirred for 1 h at 0° C. under a nitrogen atmosphere. The mixture was acidified to pH 5 with HCl (aq.). The resulting mixture was diluted with water (7 mL). The resulting mixture was extracted with EtOAc (3×15 mL). The combined organic layers were washed with brine (1×40 mL) and dried over anhydrous Na₂SO₄. After filtration, the filtrate was concentrated under reduced pressure. The crude product (50 mg) was purified by prep-HPLC with the following conditions: (column: XBridge Shield RP18 OBD column: 30*150 mm, 5 μm; mobile phase A: water (10 mmol/L NH₄HCO₃+0.1% NH₃·H₂O), mobile phase B: I; flow rate: 60 mL/min; gradient: 33% B to 50% B in 8 min, 50% B; wavelength: 220/254 nm; RT1(min): 7.92; injection volume: 1.4 mL; number of runs: 3) to afford N-(5-cyano-6-(2H-1,2,3-triazol-2-yl)pyridin-3-yl)-4-cyclopropyl-3-(imidazo[1,2-a]pyridin-5-yl)isothiazole-5-carboxamide (1-131, 5.9 mg, 8.15%) as a brown-yellow solid. LCMS (ES, m/z): [M+H]⁺⁼454; ¹H NMR (400 MHz, DMSO-d₆) δ 0.52-0.53 (m, 2H), 1.10-1.12 (m, 2H), 2.25-2.32 (m, 1H), 7.18 (t, J=6.8 Hz, 1H), 7.50 (t, J=8.0 Hz, 1H), 7.80 (d, J=8.8 Hz, 1H), 8.32 (s, 2H), 8.56 (s, 1H), 8.86 (d, J=2.4 Hz, 1H), 9.07 (s, 1H), 9.68 (d, J=6.8 Hz, 1H), 11.36 (s, 1H).

### EXAMPLE 220—SYNTHESIS OF 4-CYCLOPROPYL-3-(IMIDAZO[1,2-A]PYRIDIN-5-YL)-N-(2-(TRIFLUOROMETHYL)PYRIDIN-4-YL)ISOTHIAZOLE-5-CARBOXAMIDE (I-132)

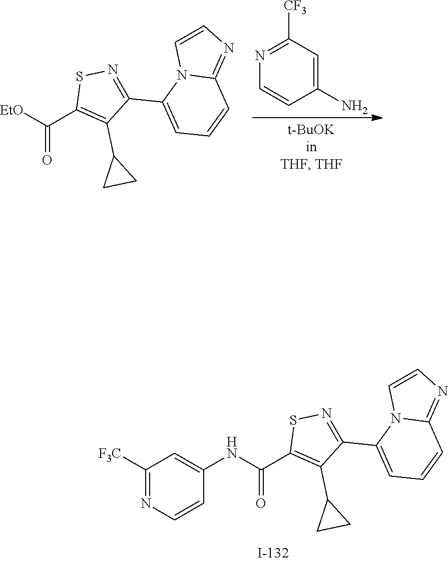

A mixture of ethyl 4-cyclopropyl-3-(imidazo[1,2-a]pyridin-5-yl)isothiazole-5-carboxylate (29 mg, 0.179 mmol, 1.12 equiv), t-BuOK (54 mg, 0.481 mmol, 3.02 equiv) in THF (2 mL) was stirred for 1 h at 0° C. under a nitrogen atmosphere. The mixture was acidified to pH 4 with HCl (aq.). The resulting mixture was diluted with water (10 mL). The resulting mixture was extracted with EtOAc (3×10 mL). The combined organic layers were washed with brine (1×30 mL) and dried over anhydrous Na₂SO₄. After filtration, the filtrate was concentrated under reduced pressure. The crude product (50 mg) was purified by prep-HPLC with the following conditions: (column: YMC-Actus Triart C18 ExRS, 30*150 mm, 5 μm; mobile phase A: water (10 mmol/L NH₄HCO₃), mobile phase B: I; flow rate: 60 mL/min; gradient: 52% B to 67% B in 8 min, 67% B; wavelength: 254 nm; RT1(min): 6.23) to afford 4-cyclopropyl-3-(imidazo[1,2-a]pyridin-5-yl)-N-(2-(trifluoromethyl)pyridin-4-yl)isothiazole-5-carboxamide (I-132, 14.4 mg, 20.93%) as an off-white solid. LCMS (ES, m/z): [M+H]⁺ =430; ¹H NMR (400 MHz, DMSO-d₆) δ 0.48-0.50 (m, 2H), 1.55-1.57 (m, 2H), 2.25-2.32 (m, 1H), 7.22(t, J=7.6 Hz, 1H), 7.52 (t, J=7.6 Hz, 1H), 7.80 (d, J=9.2 Hz, 1H), 7.95 (d, J=4.4 Hz, 1H), 8.22 (d, J=1.6 Hz, 1H), 8.55 (s, 1H), 8.74 (d, J=5.6 Hz, 1H), 9.67 (d, J=6.8 Hz, 1H), 11.48 (s, 1H).

### EXAMPLE 221—SYNTHESIS OF N-(3-CHLORO-4-(3-HYDROXYAZETIDINE-1-CARBONYL)PHENYL)-4-CYCLOPROPYL-3-(QUINOLIN-5-YL)ISOTHIAZOLE-5-CARBOXAMIDE (I-133)

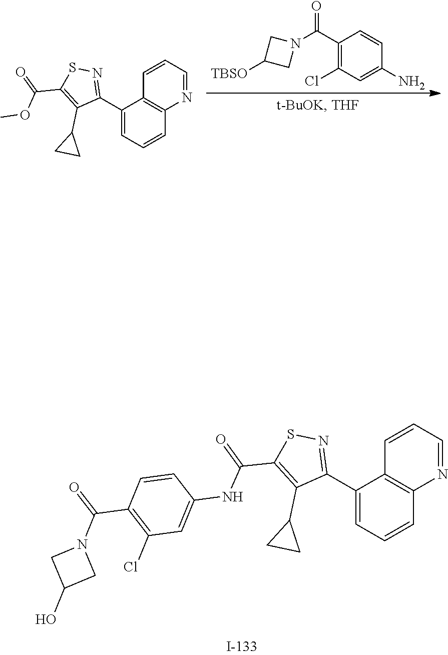 [SMILES: COC(=O)c1snc(-c2cccc3ncccc23)c1C1CC1 ; CC(C)(C)[Si](C)(C)OC1CN(C(=O)c2ccc(N)cc2Cl)C1 ; O=C(Nc1ccc(C(=O)N2CC(O)C2)c(Cl)c1)c1snc(-c2cccc3ncccc23)c1C1CC1]

To a stirred solution of methyl 4-cyclopropyl-3-(quinolin-5-yl)isothiazole-5-carboxylate (37 mg, 0.119 mmol, 1 equiv) and 4-{3-[(tert-butyldimethylsilyl)oxy]azetidine-1-carbonyl}-3-chloroaniline (60.96 mg, 0.178 mmol, 1.5 equiv) in THF (4 mL) was added t-BuOK in THF solution (1M, 0.27 mL, 2.2 equiv) dropwise at 0° C. under an N2 atmosphere. The solution was stirred at room temperature for 4 h. The reaction was quenched by the addition of water (10 mL) at 0° C. The crude product was purified by prep-HPLC with the following conditions: (column: Xbridge Prep OBD Cis column: 30*150 mm, 5 m; flow rate: 60 mL/min; gradient: 32% B to 62% B in 7 min, 62% B; wavelength: 220 nm; RT1(min): 6.47) to afford N-(3-chloro-4-(3-hydroxyazetidine-1-carbonyl)phenyl)-4-cyclopropyl-3-(quinolin-5-yl)isothiazole-5-carboxamide (I-133, 8.0 mg, 13.04%) as a white solid. LCMS (ES, m/z): [M+1]⁻=505; ¹H NMR (400 MHz, DMSO-d, ppm) δ 0.25 (d, J=4.0 Hz, 2H), 0.53 (dd, J=2.0, 8.0 Hz, 2H), 1.90-2.01 (m, 1H), 3.62-3.87 (m, 2H), 4.02 (t, J=4.0 Hz, 1H), 4.2-4.25 (m, 1H), 4.50 (s, 1H), 5.82 (d, J=6.0 Hz, 1H), 7.47 (t, J=8.8 Hz, 1H), 7.57-7.61 (m, 1H), 7.69 (s, 1H), 7.75 (d, J=6.8 Hz, 1H), 7.90-7.97 (m, 2H), 8.14-8.20 (m, 2H), 8.98 (dd, J=1.2, 4.0 Hz, 1H), 10.96 (s, 1H).

### EXAMPLE 222—SYNTHESIS OF N-(5-CYANO-6-(2H-1,2,3-TRIAZOL-2-YL) PYRIDIN-3-YL)-4-CYCLOPROPYL-3-(1-METHYL-1,2,3,6-TETRAHYDRO-PYRIDIN-4-YL)ISOTHIAZOLE-5-CARBOXAMIDE (I-134)

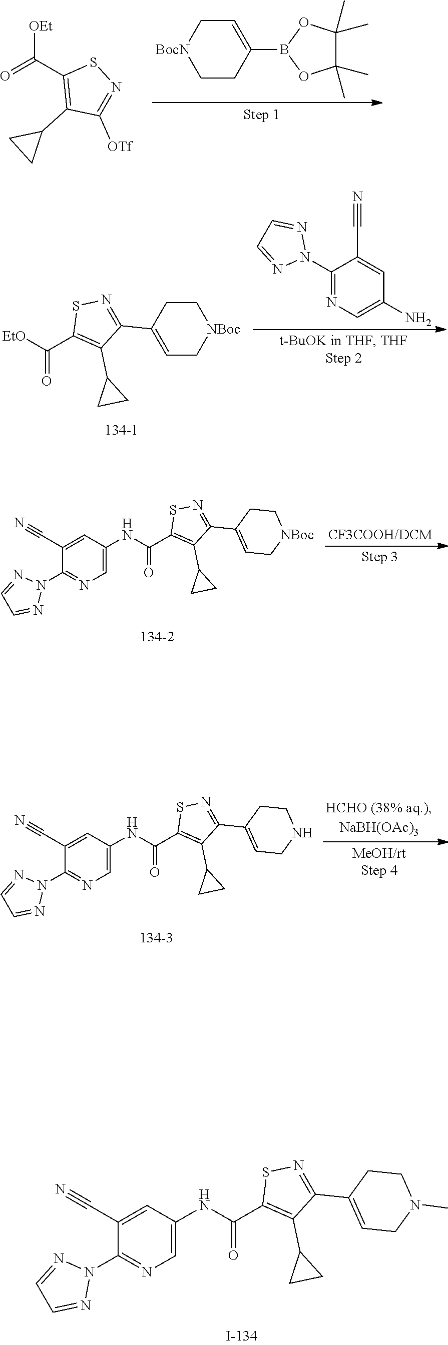

Step 1: Synthesis of 134-1: A mixture of ethyl 4-cyclopropyl-3-(trifluoromethanesulfonyloxy)-1,2-thiazole-5-carboxylate (400 mg, 1.158 mmol, 1 equiv), tert-butyl 4-(4,4,5,5-tetramethyl-1,3,2-dioxaborolan-2-yl)-3,6-dihydro-2H-pyridine-1-carboxylate (401 mg, 1.297 mmol, 1.12 equiv), XPhos Pd G3 (196 mg, 0.232 mmol, 0.20 equiv), XPhos (133 mg, 0.279 mmol, 0.24 equiv), and Cs₂CO₃ (1132 mg, 3.474 mmol, 3.00 equiv) in dioxane (5 mL) was stirred overnight at 60° C. under a nitrogen atmosphere. The residue was dissolved in water (10 mL). The resulting mixture was extracted with EtOAc (3×20 mL). The combined organic layers were washed with brine (1×50 mL) and dried over anhydrous Na₂SO₄. After filtration, the filtrate was concentrated under reduced pressure. The residue was purified by prep-TLC (PE/EA 5:1) to afford tert-butyl 4-[4-cyclopropyl-5-(ethoxycarbonyl)-1,2-thiazol-3-yl]-3,6-dihydro-2H-pyridine-1-carboxylate (134-1, 300 mg, 62.95%) as an off-white solid.

Step 2: Synthesis of 134-2: A mixture of tert-butyl 4-[4-cyclopropyl-5-(ethoxycarbonyl)-1,2-thiazol-3-yl]piperidine-1-carboxylate (180 mg, 0.473 mmol, 1 equiv), 5-amino-2-(1,2,3-triazol-2-yl)pyridine-3-carbonitrile (108 mg, 0.580 mmol, 1.23 equiv), and t-BuOK (162 mg, 1.444 mmol, 3.05 equiv) in THF (3 mL) was stirred for 1 h at 0° C. under a nitrogen atmosphere. The mixture was acidified to pH 5 with HCl (aq.). The residue was dissolved in water (7 mL). The resulting mixture was extracted with EtOAc (3×15 mL). The combined organic layers were washed with brine (1×7 mL) and dried over anhydrous Na₂SO₄. After filtration, the filtrate was concentrated under reduced pressure. The residue was purified by prep-TLC (PE/EA 1:1) to afford tert-butyl4-(5-((5-cyano-6-(2H-1,2,3-triazol-2-yl)pyridin-3-yl)carbamoyl)-4-cyclopropylisothiazol-3-yl)-3,6-dihydropyridine-1(2H)-carboxylate (134-2, 150 mg, 52.99%) as an off-white solid.

Step 3: Synthesis of 134-3: To a solution of tert-butyl 4-(5-((5-cyano-6-(2H-1,2,3-triazol-2-yl)pyridin-3-yl)carbamoyl)-4-cyclopropylisothiazol-3-yl)-3,6-dihydropyridine-1(2H)-carboxylate (150 mg, 0.290 mmol, 1 equiv) in DCM (3 mL) was added TFA (1 mL). The mixture was stirred at room temperature for 4 h. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. This resulted in N-[5-cyano-6-(1,2,3-triazol-2-yl)pyridin-3-yl]-4-cyclopropyl-3-(piperidin-4-yl)-1,2-thiazole-5-carboxamide (134-3, 80 mg, 61.67%) as an off-white solid.

Step 4: Synthesis of N-(5-cyano-6-(2H-1,2,3-triazol-2-yl)pyridin-3-yl)-4-cyclopropyl-3-(1-methyl-1,2,3,6-tetrahydropyridin-4-yl)isothiazole-5-carboxamide (I-134): A solution of N-[5-cyano-6-(1,2,3-triazol-2-yl)pyridin-3-yl]-4-cyclopropyl-3-(piperidin-4-yl)-1,2-thiazole-5-carboxamide (80 mg, 0.190 mmol, 1 equiv), HCHO (17 mg, 0.573 mmol, 3 equiv) and NaBH(OAc)₃ (13 mg, 0.573 mmol, 3 equiv) in MeOH (10 mL) was stirred overnight at room temperature under a nitrogen atmosphere. The resulting mixture was diluted with water (10 mL). The resulting mixture was extracted with EtOAc (3×20 mL). The combined organic layers were washed with brine (1×50 mL) and dried over anhydrous Na₂SO₄. After filtration, the filtrate was concentrated under reduced pressure. The crude product (50 mg) was purified by prep-HPLC with the following conditions: (column: XBridge Shield RP18 OBD column: 30*150 mm, 5 m; mobile phase A: water (10 mmol/L NH₄HCO₃+0.1% NH₃·H₂O), mobile phase B: ACN; flow rate: 60 mL/min; gradient: 33% B to 50% B in 8 min, 50% B; wavelength: 220/254 nm; RT1(min): 7.92; injection volume: 1.4 mL; number of runs: 3) to afford N-[5-cyano-6-(1,2,3-triazol-2-yl) pyridin-3-yl]-4-cyclopropyl-3-(1-methyl-3,6-dihydro-2H-pyridin-4-yl)-1,2-thiazole-5-carboxamide (I-134, 0.6 mg, 0.63%) as an off-white solid. LCMS (ES, m/z): [M+H]⁺=433; ¹H NMR (400 MHz, DMSO-d₆) δ 0.38-0.61 (m, 2H), 0.89-1.10 (m, 2H), 1.98-2.11 (m, 1H), 2.31 (s, 3H), 2.59-2.62 (m, 4H), 3.09-3.14 (m, 2H), 6.50 (s, 1H), 8.19 (s, 1H), 8.31 (s, 2H), 8.81 (d, J=2.8 Hz, 1H), 9.06 (d, J=2.4 Hz, 1H), 11.26 (br s, 1H).

### EXAMPLE 223—SYNTHESIS OF 3-(2-CHLOROPHENYL)-4-CYCLOPROPYL-N-(2-(TRIFLUOROMETHYL)PYRIDIN-4-YL)ISOTHIAZOLE-5-CARBOXAMIDE (I-135)

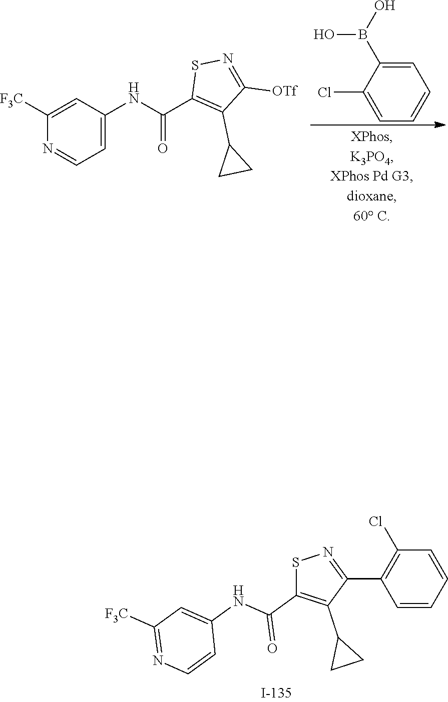 [SMILES: O=C(Nc1ccnc(C(F)(F)F)c1)c1snc(OS(=O)(=O)C(F)(F)F)c1C1CC1 ; OB(O)c1ccccc1Cl ; O=C(Nc1ccnc(C(F)(F)F)c1)c1snc(-c2ccccc2Cl)c1C1CC1]

A mixture of 4-cyclopropyl-5-{[2-(trifluoromethyl)pyridin-4-yl]carbamoyl}-1,2-thiazol-3-yl trifluoromethanesulfonate (100 mg, 0.217 mmol, 1 equiv), 2-chlorophenylboronic acid (67.79 mg, 0.434 mmol, 2 equiv), K₃PO₄ (138.03 mg, 0.651 mmol, 3 equiv), XPhos Pd G3 (18.35 mg, 0.022 mmol, 0.1 equiv) and XPhos (15.50 mg, 0.033 mmol, 0.15 equiv) in dioxane (2 mL) was stirred for 2 h at 60° C. under a nitrogen atmosphere. The resulting mixture was filtered, and the filter cake was washed with CH₂Cl₂ (2×10 mL). The filtrate was concentrated under reduced pressure and was diluted with water (30 mL). The resulting mixture was extracted with EtOAc (3×50 mL). The combined organic layers were washed with brine (1×10 mL) and dried over anhydrous Na₂SO₄. After filtration, the filtrate was concentrated under reduced pressure. The residue was purified by prep-HPLC with the following conditions: (column: XBridge Prep OBD C18 column: 30*150 mm, 5 μm; mobile phase A: water (10 mmol/L NH₄HCO₃+0.1% NH₃·H₂O), mobile phase B: ACN; flow rate: 60 mL/min; gradient: 50% B to 70% B in 8 min, 70% B; wavelength: 220 nm; RT1(min): 7.90; number of runs: 2) to afford 3-(2-chlorophenyl)-4-cyclopropyl-N-[2-(trifluoromethyl)pyridin-4-yl]-1,2-thiazole-5-carboxamide (I-135, 35.6 mg, 38.63%) as an off-white solid. LCMS (ES, m/z): [M+H]⁺=424; ¹H NMR (400 MHz, DMSO-d₆) δ 0.28-0.37 (m, 2H), 0.58-0.69 (m, 2H), 1.99 (tt, J=8.6, 5.6 Hz, 1H), 7.44-7.68 (m, 4H), 7.97 (dd, J=5.6, 2.0 Hz, 1H), 8.23 (d, J=2.0 Hz, 1H), 8.72 (d, J=5.6 Hz, 1H), 11.32 (s, 1H).

### EXAMPLE 224—SYNTHESIS OF 3-(4-CHLOROPHENYL)-4-CYCLOPROPYL-N-(2-(TRIFLUOROMETHYL)PYRIDIN-4-YL)ISOTHIAZOLE-5-CARBOXAMIDE (I-136)

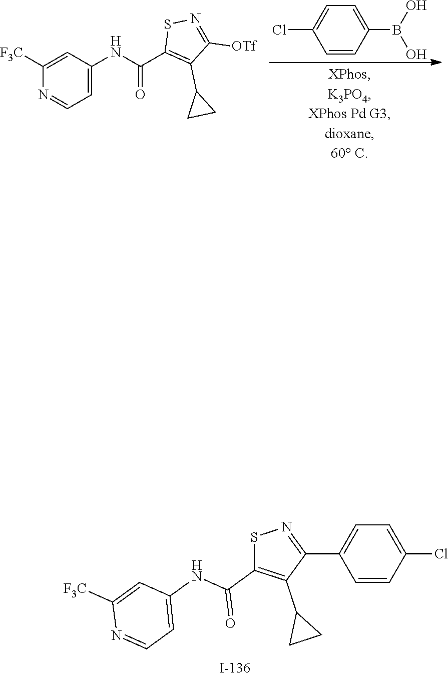 [SMILES: O=C(Nc1ccnc(C(F)(F)F)c1)c1snc(OS(=O)(=O)C(F)(F)F)c1C1CC1 ; OB(O)c1ccc(Cl)cc1 ; O=C(Nc1ccnc(C(F)(F)F)c1)c1snc(-c2ccc(Cl)cc2)c1C1CC1]

To a stirred solution of 4-cyclopropyl-5-{[2-(trifluoromethyl)pyridin-4-yl]carbamoyl}-1,2-thiazol-3-yl trifluoromethanesulfonate (100 mg, 0.217 mmol, 1 equiv), K₃PO₄ (138.03 mg, 0.651 mmol, 3 equiv) and (4-chlorophenyl)boronic acid (50.84 mg, 0.326 mmol, 1.5 equiv) in dioxane (2 mL) were added XPhos (10.33 mg, 0.022 mmol, 0.1 equiv) and XPhos Pd G3 (18.35 mg, 0.022 mmol, 0.1 equiv) in portions. The resulting mixture was stirred for 2 h at 60° C. under a nitrogen atmosphere. The reaction was quenched with water at room temperature. The resulting mixture was extracted with EtOAc (2×40 mL). The combined organic layers were washed with brine (30 mL) and dried over anhydrous Na₂SO₄. After filtration, the filtrate was concentrated under reduced pressure. The crude product (90 mg) was purified by prep-HPLC with the following conditions: (column: XBridge Shield RP18 OBD column: 30*150 mm, 5 m; mobile phase A: water (10 mmol/L NH₄HCO₃+0.1% NH₃·H₂O), mobile phase B: ACN; flow rate: 60 mL/min; gradient: 57% B to 75% B in 8 min, 75% B; wavelength: 220/254 nm; RT1(min): 7.43; injection volume: 0.5 mL; number of runs: 4) to afford 3-(4-chlorophenyl)-4-cyclopropyl-N-[2-(trifluoromethyl)pyridin-4-yl]-1,2-thiazole-5-carboxamide (1-136, 38.1 mg, 41.39%) as a light yellow solid. LCMS (ES, m/z): [M+H]⁺=424; ¹H NMR (400 MHz, DMSO-d₆) δ 0.27-0.36 (m, 2H), 0.81-0.90 (m, 2H), 2.21 (m, J=13.8, 8.6, 5.6 Hz, 1H), 7.56-7.64 (m, 2H), 7.84-7.92 (m, 2H), 7.93 (dd, J=5.6, 2.0 Hz, 1H), 8.22 (d, J=2.0 Hz, 1H), 8.72 (d, J=5.6 Hz, 1H), 11.36 (s, 1H).

### EXAMPLE 225—SYNTHESIS OF 4-CYCLOPROPYL-3-(4-(TRIFLUOROMETHYL) PHENYL)-N-(2-(TRIFLUOROMETHYL)PYRIDIN-4-YL)ISOTHIAZOLE-5-CARBOXAMIDE (I-137)

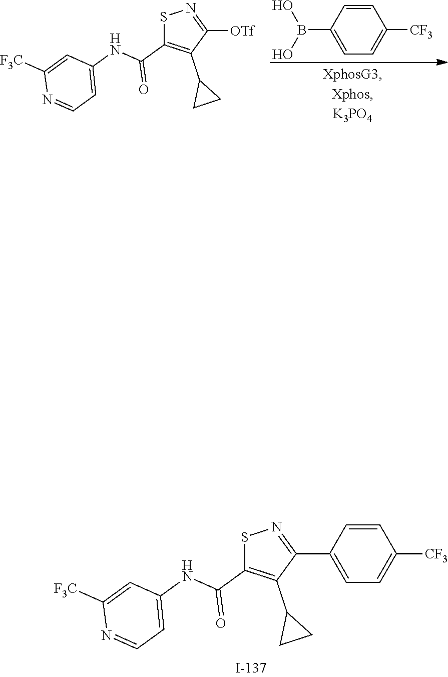 [SMILES: O=C(Nc1ccnc(C(F)(F)F)c1)c1snc(OS(=O)(=O)C(F)(F)F)c1C1CC1 ; OB(O)c1ccc(C(F)(F)F)cc1 ; O=C(Nc1ccnc(C(F)(F)F)c1)c1snc(-c2ccc(C(F)(F)F)cc2)c1C1CC1]

A solution of 4-cyclopropyl-5-{[2-(trifluoromethyl)pyridin-4-yl]carbamoyl}-1,2-thiazol-3-yl trifluoromethanesulfonate (100 mg, 0.217 mmol, 1 equiv), (4-(trifluoromethyl)phenyl)boronic acid (62 mg, 0.326 mmol, 1.5 equiv), XPhos Pd G3 (36 mg, 0.043 mmol, 0.2 equiv), XPhos (25 mg, 0.054 mmol, 0.25 equiv) and K₃PO₄ (138 mg, 0.651 mmol, 3 equiv) in dioxane (2 mL) was stirred for 2 h at 60° C. under a nitrogen atmosphere. The resulting mixture was diluted with water (10 mL). The resulting mixture was extracted with EtOAc (3×10 mL). The combined organic layers were washed with brine (1×30 mL) and dried over anhydrous Na₂SO₄. After filtration, the filtrate was concentrated under reduced pressure. The crude product was purified by prep-HPLC with the following conditions: column: XBridge Shield RP18 OBD column: 30*150 mm, 5 μm; mobile phase A: water (10 mmol/L NH₄HCO₃+0.1% NH₃·H₂O), mobile phase B: ACN; flow rate: 60 mL/min; gradient: 55% B to 75% B in 8 min, 75% B; wavelength: 220/254 nm; RT1(min): 7.5 to afford 4-cyclopropyl-3-[4-(trifluoromethyl)phenyl]-N-[2-(trifluoromethyl)pyridin-4-yl]-1,2-thiazole-5-carboxamide (I-137, 45.4 mg, 45.61%) as an off-white solid. LCMS (ES, m/z): [M+H]⁺=458; ¹H NMR (DMSO-d₆, 400 MHz, ppm): δ 0.25-0.38 (m, 2H), 0.79-0.93 (m, 2H), 2.20-2.31 (m, 1H), 7.86-7.98 (m, 3H), 8.08 (s, 1H), 8.10 (d, J=8.0 Hz, 1H), 8.23 (d, J=2.0 Hz, 1H), 8.73 (d, J=5.6 Hz, 1H), 11.39 (s, 1H).

### EXAMPLE 226—SYNTHESIS OF 4-CYCLOPROPYL-3-(3-(TRIFLUOROMETHYL) PHENYL)-N-(2-(TRIFLUOROMETHYL)PYRIDIN-4-YL)ISOTHIAZOLE-5-CARBOXAMIDE (1-138)

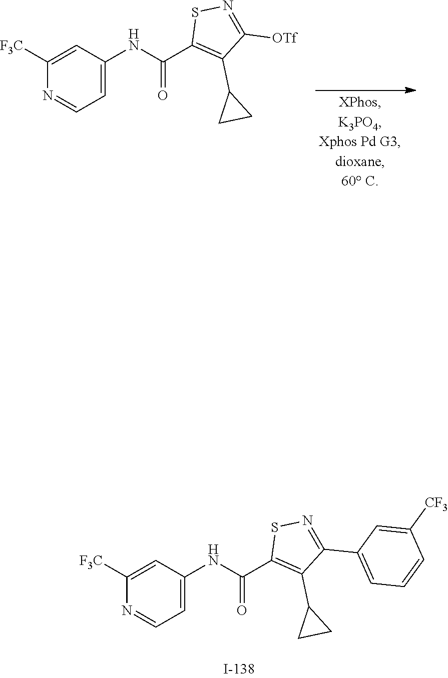 [SMILES: O=C(Nc1ccnc(C(F)(F)F)c1)c1snc(OS(=O)(=O)C(F)(F)F)c1C1CC1 ; O=C(Nc1ccnc(C(F)(F)F)c1)c1snc(-c2cccc(C(F)(F)F)c2)c1C1CC1]

A solution of 4-cyclopropyl-5-{[2-(trifluoromethyl)pyridin-4-yl]carbamoyl}-1,2-thiazol-3-yl trifluoromethanesulfonate (100 mg, 0.217 mmol, 1 equiv) in dioxane (2 mL) was treated with [3-(trifluoromethyl)phenyl]boronic acid (61.75 mg, 0.326 mmol, 1.5 equiv) for 10 min at room temperature under a nitrogen atmosphere followed by the addition of XPhos (20.67 mg, 0.043 mmol, 0.2 equiv), XPhos Pd G3 (73.39 mg, 0.087 mmol, 0.4 equiv) and K₃PO₄ (138.03 mg, 0.651 mmol, 3 equiv) in portions at room temperature. The resulting mixture was stirred for an additional 2 h at 60° C. The residue was purified by reversed-phase flash chromatography with the following conditions: column: C18 silica gel; mobile phase: MeCN in water (0.1% TFA); 0% to 100% gradient in 25 min; detector: UV 254 nm. This resulted in 4-cyclopropyl-3-(3-(trifluoromethyl)phenyl)-N-(2-(trifluoromethyl)pyridin-4-yl)isothiazole-5-carboxamide (1-138, 21.3 mg, 21.27%) as a white solid. LCMS (ES, m/z): [M+H]⁺=458; ¹H NMR (400 MHz, DMSO-d₆): δ 0.32 (d, J=5.2 Hz, 2H), 0.85 (d, J=8.0 Hz, 2H), 2.24-2.32 (m, 1H), 7.81 (d, J=8.0 Hz, 1H), 7.90 (d, J=6.8 Hz, 1H), 7.95 (d, J=5.2 Hz, 1H), 8.17-8.22 (m, 2H), 8.15 (s, 1H), 8.74 (d, J=5.2 Hz, 1H), 11.37 (s, 1H).

### EXAMPLE 227—SYNTHESIS OF 3-(3-CHLOROPHENYL)-4-CYCLOPROPYL-N-(2-(TRIFLUOROMETHYL)PYRIDIN-4-YL)ISOTHIAZOLE-5-CARBOXAMIDE (I-139)

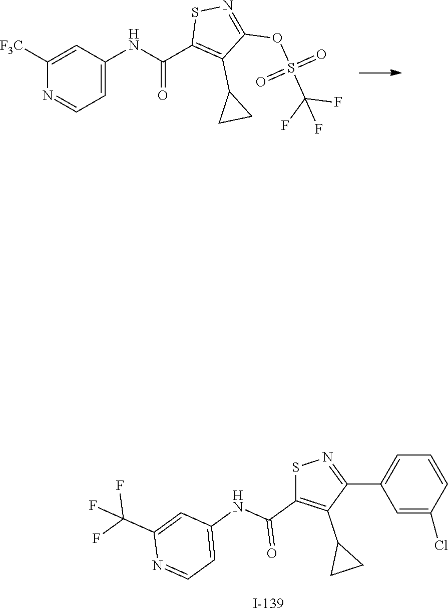 [SMILES: O=C(Nc1ccnc(C(F)(F)F)c1)c1snc(OS(=O)(=O)C(F)(F)F)c1C1CC1 ; O=C(Nc1ccnc(C(F)(F)F)c1)c1snc(-c2cccc(Cl)c2)c1C1CC1]

A solution of 4-cyclopropyl-5-{[2-(trifluoromethyl)pyridin-4-yl]carbamoyl}-1,2-thiazol-3-yl trifluoromethanesulfonate (100 mg, 0.217 mmol, 1 equiv) in 1,4-dioxane (2 mL) was treated with (3-chlorophenyl)boronic acid (50.84 mg, 0.326 mmol, 1.5 equiv) for 5 min at room temperature under a nitrogen atmosphere followed by the addition of XPhos (20.67 mg, 0.043 mmol, 0.2 equiv), XPhos Pd G3 (73.39 mg, 0.087 mmol, 0.4 equiv) and K₃PO₄ (138.03 mg, 0.651 mmol, 3 equiv) in portions at room temperature. The resulting mixture was stirred for an additional 2 h at 60° C. The residue was purified by reversed-phase flash chromatography with the following conditions: column: C18 silica gel; mobile phase: MeCN in water (0.1% TFA); 0% to 100% gradient in 25 min; detector: UV 254 nm. This resulted in 3-(3-chlorophenyl)-4-cyclopropyl-N-(2-(trifluoromethyl)pyridin-4-yl)isothiazole-5-carboxamide (I-139, 36.6 mg, 39.04%) as a white solid. LCMS (ES, m/z): [M+H]⁺=424; ¹H NMR (400 MHz, DMSO-d₆): δ 0.29-0.38 (m, 2H), 0.80-0.91 (m, 2H), 2.22-2.25 (m, 1H), 7.54-7.63 (m, 2H), 7.80-7.89 (m, 2H), 7.95 (dd, J=5.6, 2.0 Hz, 1H), 8.23 (d, J=2.0 Hz, 1H), 8.73 (d, J=5.6 Hz, 1H), 11.38 (s, 1H).

### EXAMPLE 228—SYNTHESIS OF 4-CYCLOPROPYL-3-(8-METHYLQUINOLIN-5-YL)-N-(2-(TRIFLUOROMETHYL)PYRIDIN-4-YL)ISOTHIAZOLE-5-CARBOXAMIDE (I-140)

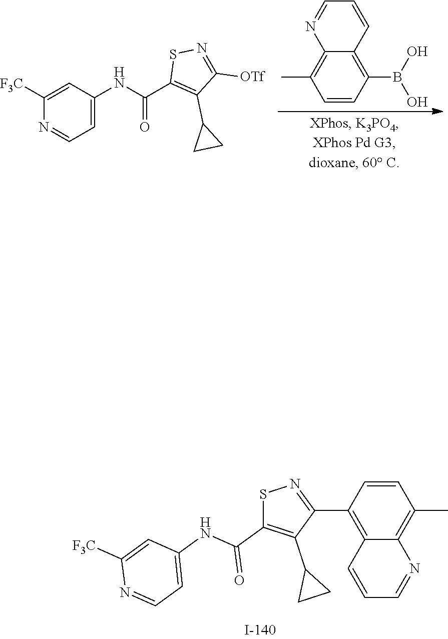

A stirred solution of 4-cyclopropyl-5-{[2-(trifluoromethyl)pyridin-4-yl]carbamoyl}-1,2-thiazol-3-yl trifluoromethanesulfonate (100 mg, 0.217 mmol, 1 equiv), (8-methylquinolin-5-yl)boronic acid (60.80 mg, 0.326 mmol, 1.5 equiv) and K₃PO₄ (138.03 mg, 0.651 mmol, 3 equiv) in dioxane (2 mL) were added XPhos (10.33 mg, 0.022 mmol, 0.1 equiv) and XPhos Pd G3 (18.35 mg, 0.022 mmol, 0.1 equiv) in portions. The resulting mixture was stirred for 2 h at 60° C. under a nitrogen atmosphere. The reaction was quenched with water at room temperature. The resulting mixture was extracted with EtOAc (2×40 mL). The combined organic layers were washed with brine (40 mL) and dried over anhydrous Na₂SO₄. After filtration, the filtrate was concentrated under reduced pressure. The crude product (70 mg) was purified by prep-HPLC with the following conditions: (column: XBridge Prep OBD C18 column: 30*150 mm, 5 μm; mobile phase A: water (10 mmol/L NH₄HCO₃+0.1% NH₃·H₂O), mobile phase B: ACN; flow rate: 60 mL/min; gradient: 45% B to 70% B in 8 min, 70% B; wavelength: 220 nm; RT1(min): 7.57; number of runs: 1) to afford 4-cyclopropyl-3-(8-methylquinolin-5-yl)-N-[2-(trifluoromethyl)pyridin-4-yl]-1,2-thiazole-5-carboxamide (I-140, 27.2 mg, 27.58%) as a white solid. LCMS (ES, m/z): [M+H]⁺=455; ¹H NMR (400 MHz, DMSO-d₆) δ 0.19-0.28 (m, 2H), 0.47-0.58 (m, 2H), 1.93 (tt, J=8.6, 5.6 Hz, 1H), 2.82 (d, J=1.0 Hz, 3H), 7.59 (dd, J=8.6, 4.2 Hz, 1H), 7.64 (d, J=7.4 Hz, 1H), 7.78 (dd, J=7.4, 1.2 Hz, 1H), 7.96 (dd, J=5.6, 2.0 Hz, 1H), 8.13 (dd, J=8.6, 1.8 Hz, 1H), 8.24 (d, J=2.0 Hz, 1H), 8.74 (d, J=5.6 Hz, 1H), 9.01 (dd, J=4.2, 1.8 Hz, 1H), 11.38 (s, 1H).

### EXAMPLE 229—SYNTHESIS OF 3-(3-ACETAMIDOPHENYL)-4-CYCLOPROPYL-N-(2-(TRIFLUOROMETHYL)PYRIDIN-4-YL)ISOTHIAZOLE-5-CARBOXAMIDE (I-141)

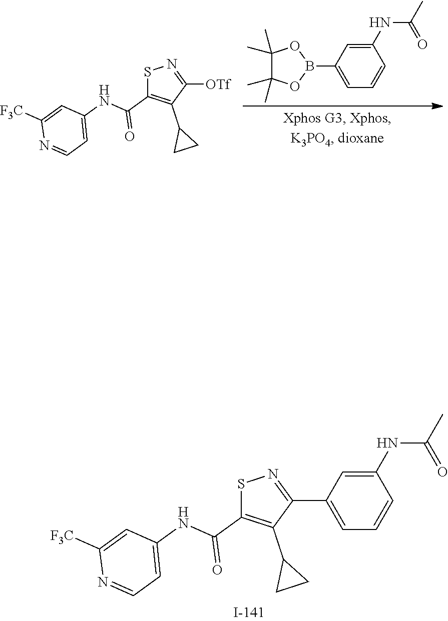 [SMILES: O=C(Nc1ccnc(C(F)(F)F)c1)c1snc(OS(=O)(=O)C(F)(F)F)c1C1CC1 ; CC(=O)Nc1cccc(B2OC(C)(C)C(C)(C)O2)c1 ; CC(=O)Nc1cccc(-c2nsc(C(=O)Nc3ccnc(C(F)(F)F)c3)c2C2CC2)c1]

To a stirred solution of 4-cyclopropyl-5-{[2-(trifluoromethyl)pyridin-4-yl]carbamoyl}-1,2-thiazol-3-yl trifluoromethanesulfonate (100 mg, 0.217 mmol, 1 equiv) and N-[3-(4,4,5,5-tetramethyl-1,3,2-dioxaborolan-2-yl)phenyl]acetamide (85 mg, 0.326 mmol, 1.5 equiv) in dioxane (2 mL) was added K₃PO₄ (138 mg, 0.651 mmol, 3 equiv) and XPhos Pd G3 (18 mg, 0.022 mmol, 0.1 equiv). The resulting mixture was stirred for 2 h at 60° C. under a nitrogen atmosphere. The resulting mixture was diluted with water (20 mL). The resulting mixture was extracted with EtOAc (3×20 mL). The combined organic layers were washed with brine (1×40 mL) and dried over anhydrous Na₂SO₄. After filtration, the filtrate was concentrated under reduced pressure. The crude product (70 mg) was purified by prep-HPLC with the following conditions: (column: XBridge Shield RP18 OBD column: 30*150 mm, 5 μm; mobile phase A: water (10 mmol/L NH₄HCO₃+0.1% NH₃·H₂O), mobile phase B: ACN; flow rate: 60 mL/min; gradient: 37% B to 59% B in 8 min, 59% B; wavelength: 220/254 nm; RT1(min): 7.55; injection volume: 1 mL; number of runs: 2) to afford 4-cyclopropyl-3-(3-acetamidophenyl)-N-[2-(trifluoromethyl)pyridin-4-yl]-1,2-thiazole-5-carboxamide (I-141, 15.1 mg, 15.29%) as a white solid. LCMS (ES, m/z): [M+1]⁺=447; ¹H NMR (400 MHz, DMSO-d₆) δ 0.37-0.28 (m, 2H), 0.90-0.80 (m, 2H), 2.08 (s, 3H), 2.16-2.24 (m, 1H), 7.44 (t, J=8.0 Hz, 1H), 7.51 (d, J=8.0 Hz, 1H), 7.64 (d, J=8.0 Hz, 1H), 7.94 (dd, J=2.0, 5.6 Hz, 1H), 8.21 (s, 1H), 8.22 (d, J=2.0 Hz, 1H), 8.72 (d, J=5.6 Hz, 1H), 10.10 (s, 1H), 11.36 (s, 1H).

### EXAMPLE 230—SYNTHESIS OF 3-(BENZO[D]THIAZOL-5-YL)-4-CYCLOPROPYL-N-(2-(TRIFLUOROMETHYL)PYRIDIN-4-YL)ISOTHIAZOLE-5-CARBOXAMIDE (I-142)

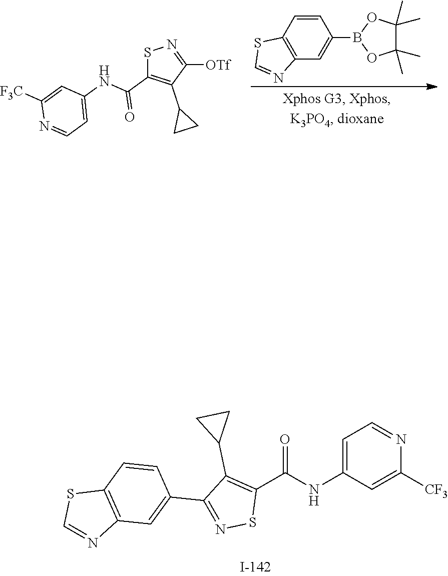 [SMILES: O=C(Nc1ccnc(C(F)(F)F)c1)c1snc(OS(=O)(=O)C(F)(F)F)c1C1CC1 ; CC1(C)OB(c2ccc3scnc3c2)OC1(C)C ; O=C(Nc1ccnc(C(F)(F)F)c1)c1snc(-c2ccc3scnc3c2)c1C1CC1]

To a stirred solution of 4-cyclopropyl-5-{[2-(trifluoromethyl)pyridin-4-yl]carbamoyl}-1,2-thiazol-3-yl trifluoromethanesulfonate (100 mg, 0.217 mmol, 1 equiv) and 5-(4,4,5,5-tetramethyl-1,3,2-dioxaborolan-2-yl)-1,3-benzothiazole (85 mg, 0.326 mmol, 1.5 equiv) in dioxane (5 mL) was added K₃PO₄ (138 mg, 0.651 mmol, 3 equiv) and XPhos Pd G3 (18 mg, 0.022 mmol, 0.1 equiv). The resulting mixture was stirred for 2 h at 60° C. under a nitrogen atmosphere. The resulting mixture was diluted with water (20 mL). The resulting mixture was extracted with EtOAc (3×20 mL). The combined organic layers were washed with brine (1×50 mL) and dried over anhydrous Na₂SO₄. After filtration, the filtrate was concentrated under reduced pressure. The crude product (70 mg) was purified by prep-HPLC with the following conditions: (column: XBridge Shield RP18 OBD column: 30*150 mm, 5 μm; mobile phase A: water (10 mmol/L NH₄HCO₃+0.1% NH₃·H₂O), mobile phase B: ACN; flow rate: 60 mL/min; gradient: 45% B to 67% B in 8 min, 67% B; wavelength: 220/254 nm; RT1(min): 7.38; injection volume: 1 mL; number of runs: 2) to afford 3-(1,3-benzothiazol-5-yl)-4-cyclopropyl-N-[2-(trifluoromethyl)pyridin-4-yl]-1,2-thiazole-5-carboxamide (I-142, 22.5 mg, 23.20%) as a white solid. LCMS (ES, m/z): [M+1]⁺=447; ¹H NMR (400 MHz, DMSO-d₆) δ 0.31-0.37 (m, 2H), 0.80-0.92 (m, 2H), 2.27-2.38 (m, 1H), 7.89-8.04 (m, 2H), 8.23 (d, J=1.6 Hz, 1H), 8.33 (d, J=8.4 Hz, 1H), 8.52 (d, J=1.2 Hz, 1H), 8.73 (d, J=5.6 Hz, 1H), 9.50 (s, 1H), 11.35 (s, 1H).

### EXAMPLE 231—SYNTHESIS OF 4-CYCLOPROPYL-3-(1,3-DIMETHYL-1H-PYRAZOL-4-YL)-N-(2-(TRIFLUOROMETHYL)PYRIDIN-4-YL)ISOTHIAZOLE-5-CARBOXAMIDE (1-143)

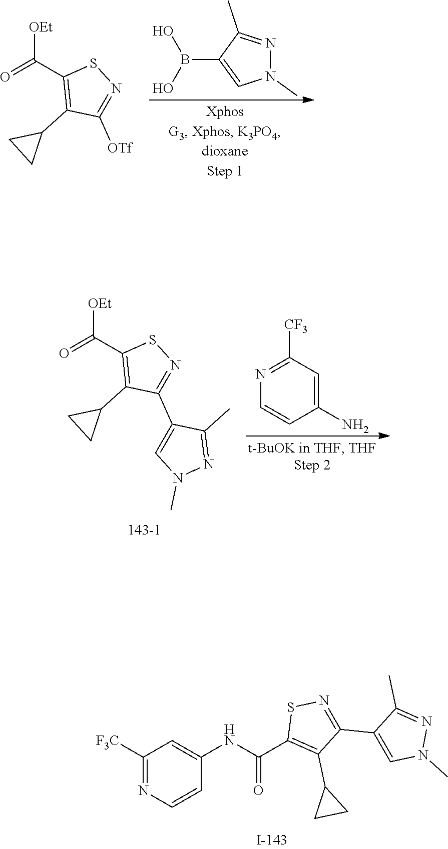

Step 1: Synthesis of 143-1: A solution of ethyl 4-cyclopropyl-3-(trifluoromethanesulfonyloxy)-1,2-thiazole-5-carboxylate (150 mg, 0.434 mmol, 1 equiv) and 1,3-dimethylpyrazol-4-ylboronic acid (73 mg, 0.522 mmol, 1.20 equiv), XPhos (42 mg, 0.088 mmol, 0.20 equiv), XPhos Pd G3 (74 mg, 0.087 mmol, 0.20 equiv) and K₃PO₄ (276 mg, 1.300 mmol, 2.99 equiv) in 1,4-dioxane (10 mL) was stirred overnight at 60° C. under a nitrogen atmosphere. The resulting mixture was diluted with water (10 mL). The resulting mixture was extracted with EtOAc (3×20 mL). The combined organic layers were washed with brine (1×50 mL) and dried over anhydrous Na₂SO₄. After filtration, the filtrate was concentrated under reduced pressure. The crude product (80 mg) was purified by prep-HPLC with the following conditions: (column: XBridge Prep OBD C18 column: 30*150 mm, 5 μm; mobile phase A: water (10 mmol/L NH₄HCO₃+0.1% NH₃·H₂O), mobile phase B: ACN; flow rate: 60 mL/min; gradient: 36% B to 65% B in 8 min, 65% B; wavelength: 220 nm; RT1(min): 7.47; number of runs: 1) to afford ethyl 4-cyclopropyl-3-(1,3-dimethylpyrazol-4-yl)-1,2-thiazole-5-carboxylate (143-1, 100 mg, 79.01%) as a light yellow oil. LCMS (ES, m/z): [M+H]⁺⁼292.

Step 2: Synthesis of 4-cyclopropyl-3-(1,3-dimethyl-1H-pyrazol-4-yl)-N-(2-(trifluoromethyl)pyridin-4-yl)isothiazole-5-carboxamide (1-143): A solution of ethyl 4-cyclopropyl-3-(1,3-dimethylpyrazol-4-yl)-1,2-thiazole-5-carboxylate (100 mg, 0.343 mmol, 1 equiv) and 2-(trifluoromethyl)pyridin-4-amine (67 mg, 0.413 mmol, 1.20 equiv) and t-BuOK (1 mL, 0.009 mmol, 0.03 equiv) in THF (10 mL) was stirred for 0.5 h at room temperature under a nitrogen atmosphere. The resulting mixture was diluted with water (5 mL). The resulting mixture was extracted with EtOAc (3×10 mL). The combined organic layers were washed with brine (1×40 mL) and dried over anhydrous Na₂SO₄. After filtration, the filtrate was concentrated under reduced pressure. The crude product (50 mg) was purified by prep-HPLC with the following conditions: (column: Xselect CSH C18 OBD column 30*150 mm 5 μm, n; mobile phase A: water (0.1% FA), mobile phase B: ACN; flow rate: 60 mL/min; gradient: 31% B to 41% B in 7 min, 41% B; wavelength: 254; 220 nm; RT1(min): 5.65, 8.32 (min)) to afford 4-cyclopropyl-3-(1,3-dimethylpyrazol-4-yl)-N-[2-(trifluoromethyl)pyridine4-yl]-1,2-thiazole-carboxamide (I-143, 49.8 mg, 35.54%) as an off-white solid. LCMS (ES, m/z): [M+H]±=408; ¹H NMR (400 MHz, DMSO-d₆) δ 0.19-0.44 (m, 2H), 0.82-1.10 (m, 2H), 1.92-2.10 (m, 1H), 2.40 (s, 3H), 3.86 (s, 3H), 7.92 (dd, J=5.6, 2.0 Hz, 1H), 8.21 (d, J=2.0 Hz, 1H), 8.29 (s, 1H), 8.71 (d, J=5.2 Hz, 1H), 11.34 (br s, 1H).

### EXAMPLE 232—SYNTHESIS OF 4-CYCLOPROPYL-3-(PYRIMIDIN-5-YL)-N-(2-(TRIFLUOROMETHYL)PYRIDIN-4-YL)ISOTHIAZOLE-5-CARBOXAMIDE (I-144)

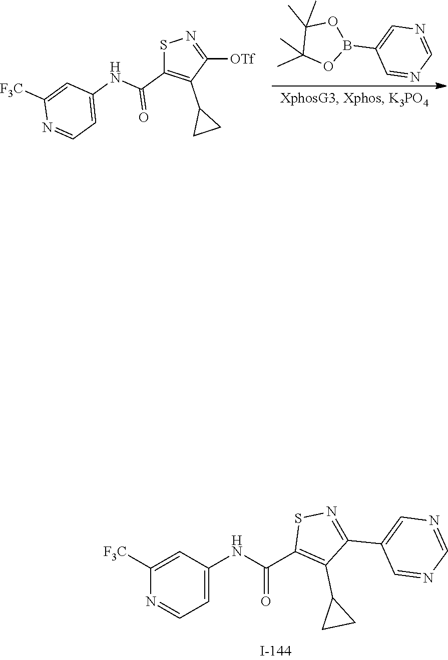 [SMILES: O=C(Nc1ccnc(C(F)(F)F)c1)c1snc(OS(=O)(=O)C(F)(F)F)c1C1CC1 ; CC1(C)OB(c2cncnc2)OC1(C)C ; O=C(Nc1ccnc(C(F)(F)F)c1)c1snc(-c2cncnc2)c1C1CC1]

A solution of 4-cyclopropyl-5-{[2-(trifluoromethyl)pyridin-4-yl]carbamoyl}-1,2-thiazol-3-yl trifluoromethanesulfonate (100 mg, 0.217 mmol, 1 equiv), 5-(4,4,5,5-tetramethyl-1,3,2-dioxaborolan-2-yl)pyrimidine (66 mg, 0.326 mmol, 1.5 equiv), XPhos Pd G3 (36 mg, 0.043 mmol, 0.2 equiv), XPhos (25.8 mg, 0.054 mmol, 0.25 equiv) and K₃PO₄ (138.0 mg, 0.651 mmol, 3 equiv) in dioxane (2 mL) was stirred for 2 h at 60° C. under a nitrogen atmosphere. The mixture was allowed to cool down to room temperature. The resulting mixture was diluted with water (10 mL). The resulting mixture was extracted with EtOAc (3×10 mL). The combined organic layers were washed with water (1×30 mL) and dried over anhydrous Na₂SO₄. After filtration, the filtrate was concentrated under reduced pressure. The crude product was purified by prep-HPLC with the following conditions: column: XBridge Prep OBD C18 column: 30*150 mm, 5 μm; mobile phase A: water (10 mmol/L NH₄HCO₃), mobile phase B: ACN; flow rate: 60 mL/min; gradient: 30% B to 55% B in 8 min, 55% B; wavelength: 254 nm; RT1(min): 7.43 to afford 4-cyclopropyl-3-(pyrimidin-5-yl)-N-[2-(trifluoromethyl)pyridin-4-yl]-1,2-thiazole-5-carboxamide (I-144, 17.2 mg, 20.26%) as an off-white solid. LCMS (ES, m/z): [M−H]⁻⁼390; ¹H NMR (400 MHz, DMSO-d₆): δ 0.31-0.40 (m, 2H), 0.79-0.97 (m, 2H), 2.20-2.40 (m, 1H), 7.95 (dd, J=2.0, 5.6 Hz, 1H), 8.22 (d, J=1.6 Hz, 1H), 8.73 (d, J=5.6 Hz, 1H), 9.29 (s, 2H), 9.31 (d, J=5.6 Hz, 1H), 11.38 (s, 1H).

### EXAMPLE 233—SYNTHESIS OF 4-CYCLOPROPYL-3-(4-(DIMETHYLAMINO) PHENYL)-N-(2-(TRIFLUOROMETHYL)PYRIDIN-4-YL)ISOTHIAZOLE-5-CARBOXAMIDE (I-145)

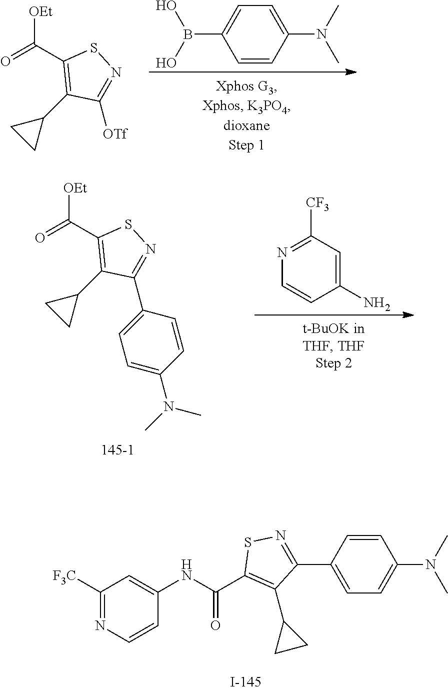 [SMILES: CCOC(=O)c1snc(OS(=O)(=O)C(F)(F)F)c1C1CC1 ; CN(C)c1ccc(B(O)O)cc1 ; CCOC(=O)c1snc(-c2ccc(N(C)C)cc2)c1C1CC1 ; Nc1ccnc(C(F)(F)F)c1 ; CN(C)c1ccc(-c2nsc(C(=O)Nc3ccnc(C(F)(F)F)c3)c2C2CC2)cc1]

Step 1: Synthesis of 145-1: A solution of ethyl 4-cyclopropyl-3-(trifluoromethanesulfonyloxy)-1,2-thiazole-5-carboxylate (150 mg, 0.434 mmol, I equiv) and 4-(dimethylamino)phenylboronic acid (80 mg, 0.485 mmol, 1.12 equiv), XPhos (42 mg, 0.088 mmol, 0.20 equiv), Xphos Pd G3 (74 mg, 0.087 mmol, 0.20 equiv) and K₃PO₄ (276 mg, 1.300 mmol, 2.99 equiv) in 1,4-dioxane (10 mL) was stirred overnight at 60° C. under a nitrogen atmosphere. The resulting mixture was diluted with water (10 mL). The resulting mixture was extracted with EtOAc (3×20 mL). The combined organic layers were washed with brine (1×40 mL) and dried over anhydrous Na₂SO₄. After filtration, the filtrate was concentrated under reduced pressure. The crude product (80 mg) was purified by prep-HPLC with the following conditions: (column: Xbridge Prep OBD C18 column: 30*150 mm, 5 μm; mobile phase A: water (10 mmol/L NH₄HCO₃+0.1% NH₃·H₂O), mobile phase B: ACN; flow rate: 60 mL/min; gradient: 36% B to 65% B in 8 min, 65% B; wavelength: 220 nm; RT1(min): 7.47; number of runs: 1) to afford ethyl 4-cyclopropyl-3-[4-(dimethylamino)phenyl]-1,2-thiazole-5-carboxylate (145-1, 90 mg, 65.48%) as a light yellow oil.

Step 2: Synthesis of 4-cyclopropyl-3-(4-(dimethylamino)phenyl)-N-(2-(trifluoromethyl)pyridine-4-yl)isothiazole-5-carboxamide (I-145): A solution of ethyl 4-cyclopropyl-3-[4-(dimethylamino)phenyl]-1,2-thiazole-5-carboxylate (100 mg, 0.316 mmol, 1 equiv) and 2-(trifluoromethyl)pyridine-4-amine (61 mg, 0.376 mmol, 1.19 equiv) and t-BuOK (106 mg, 0.945 mmol, 2.99 equiv) in THF (10 mL) was stirred for 0.5 h at room temperature under a nitrogen atmosphere. The resulting mixture was diluted with water (5 mL). The resulting mixture was extracted with EtOAc (3×10 mL). The combined organic layers were washed with brine (lx 20 mL) and dried over anhydrous Na₂SO₄. After filtration, the filtrate was concentrated under reduced pressure. The crude product (50 mg) was purified by prep-HPLC with the following conditions: (column: Xselect CSH C18 OBD column 30*150 mm 5 μm; mobile phase A: water (0.1% FA), mobile phase B: ACN; flow rate: 60 mL/min; gradient: 31% B to 41% B in 7 min, 41% B; wavelength: 254; 220 nm; RT1(min): 5.65, 8.32 (min)) to afford 4-cyclopropyl-3-[4-(dimethylamino)phenyl]-N-[2-(trifluoromethyl)pyridine-4-yl]-1,2-thiazole-5-carboxamide (I-145, 2.3 mg, 1.67%) as an off-white solid. LCMS (ES, m/z): [M+H]⁺=433; ¹H NMR (400 MHz, DMSO-d₆) δ 0.21-0.45 (m, 2H), 0.79-0.92 (m, 2H), 2.10-2.28 (m, 1H), 2.99 (s, 6H), 6.81 (d, J=8.8 Hz, 2H), 7.76 (d, J=8.8 Hz, 2H), 7.94 (d, J=4.8 Hz, 1H), 8.21 (d, J=2.0 Hz, 1H), 8.71 (d, J=5.2 Hz, 1H), 11.31 (s, 1H).

### EXAMPLE 233—SYNTHESIS OF 4-CYCLOPROPYL-3-(5-FLUOROPYRIDIN-3-YL)-N-(2-(TRIFLUOROMETHYL)PYRIDIN-4-YL)ISOTHIAZOLE-5-CARBOXAMIDE (I-146)

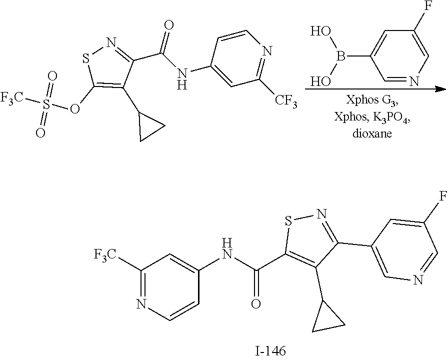 [SMILES: O=C(Nc1ccnc(C(F)(F)F)c1)c1nsc(OS(=O)(=O)C(F)(F)F)c1C1CC1 ; OB(O)c1cncc(F)c1 ; O=C(Nc1ccnc(C(F)(F)F)c1)c1snc(-c2cncc(F)c2)c1C1CC1]

A solution of 4-cyclopropyl-5-{[2-(trifluoromethyl)pyridine-4-yl]carbamoyl}-1,2-thiazol-3-yl trifluoromethanesulfonate (150 mg, 0.325 mmol, 1 equiv), 5-fluoropyridin-3-ylboronic acid (55 mg, 0.390 mmol, 1.20 equiv), Xphos (31.00 mg, 0.065 mmol, 0.2 equiv), Xphos Pd G₃ (55.04 mg, 0.065 mmol, 0.2 equiv) and K₃PO₄ (207 mg, 0.975 mmol, 3.00 equiv) in 1,4-dioxane (10 mL) was stirred overnight at 60° C. under a nitrogen atmosphere. The resulting mixture was diluted with water (10 mL). The resulting mixture was extracted with EtOAc (3×20 mL). The combined organic layers were washed with brine (1×40 mL) and dried over anhydrous Na₂SO₄. After filtration, the filtrate was concentrated under reduced pressure. The crude product (80 mg) was purified by prep-HPLC with the following conditions: (column: Xbridge Prep OBD C18 column: 30*150 mm, 5 μm; mobile phase A: water (10 mmol/L NH₄HCO₃+0.1% NH₃·H₂O), mobile phase B: ACN; flow rate: 60 mL/min; gradient: 36% B to 65% B in 8 min, 65% B; wavelength: 220 nm; RT1(min): 7.47; number of runs: 1) to afford 4-cyclopropyl-3-(5-fluoropyridin-3-yl)-N-[2-(trifluoromethyl)pyridine-4-yl]-1,2-thiazole-5-carboxamide (I-146, 15.6 mg, 11.74%) as an off-white solid. LCMS (ES, m/z): [M+H]⁺=409; ¹H NMR (400 MHz, DMSO-d₆) δ 0.34 (dd, J=5.6, 1.2 Hz, 2H), 0.82-1.02 (m, 2H), 2.23-2.33 (m, 1H), 7.94 (dd, J=5.2, 2.0 Hz, 1H), 8.11-8.21 (m, 1H), 8.22 (d, J=1.6 Hz, 1H), 8.60-8.87 (m, 2H), 8.94 (t, J=1.6 Hz, 1H), 11.41 (s, 1H).

### EXAMPLE 234—SYNTHESIS OF 4-CYCLOPROPYL-3-(2-FLUOROPHENYL)-N-(2-(TRIFLUOROMETHYL)PYRIDIN-4-YL)ISOTHIAZOLE-5-CARBOXAMIDE (I-147)

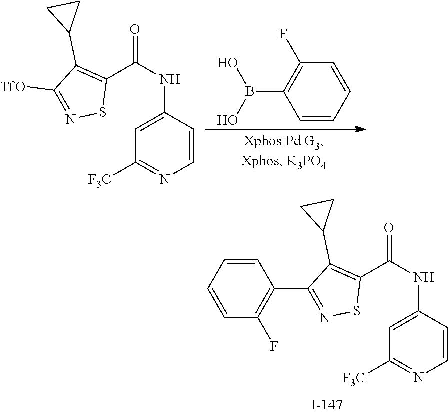

A solution of 4-cyclopropyl-5-{[2-(trifluoromethyl)pyridine-4-yl]carbamoyl}-1,2-thiazol-3-yl trifluoromethanesulfonate (150 mg, 0.325 mmol, 1 equiv), 2-fluorophenylboronic acid (55 mg, 0.393 mmol, 1.21 equiv), Xphos (31.00 mg, 0.065 mmol, 0.2 equiv), Xphos Pd G₃ (55.00 mg, 0.065 mmol, 0.20 equiv) and K₃PO₄ (207 mg, 0.975 mmol, 3.00 equiv) in 1,4-dioxane (10 mL) was stirred overnight at 60° C. under a nitrogen atmosphere. The resulting mixture was diluted with water (10 mL). The resulting mixture was extracted with EtOAc (3×20 mL). The combined organic layers were washed with brine (1×40 mL) and dried over anhydrous Na₂SO₄. After filtration, the filtrate was concentrated under reduced pressure. The crude product (80 mg) was purified by prep-HPLC with the following conditions: (column: Xbridge Prep OBD C18 column: 30*150 mm, 5 m; mobile phase A: water (10 mmol/L NH₄HCO₃+0.1% NH₃·H₂O), mobile phase B: ACN; flow rate: 60 mL/min; gradient: 36% B to 65% B in 8 min, 65% B; wavelength: 220 nm; RT1(min): 7.47; number of runs: 1) to afford 4-cyclopropyl-3-(2-fluorophenyl)-N-[2-(trifluoromethyl)pyridine-4-yl]-1,2-thiazole-5-carboxamide (1-147, 11.9 mg, 8.97%) as an off-white solid. LCMS (ES, m/z): [M+H]⁺=408; ¹H NMR (400 MHz, DMSO-d₆) δ 0.12-0.40 (m, 2H), 0.52-0.80 (m, 2H), 1.92-2.20 (m, 1H), 7.25-7.47 (m, 2H), 7.48-7.72 (m, 2H), 7.95 (dd, J=5.6, 2.0 Hz, 1H), 8.23 (d, J=2.0 Hz, 1H), 8.72 (d, J=5.6 Hz, 1H), 11.36 (s, 1H).

### EXAMPLE 235—SYNTHESIS OF 3-(4-ACETAMIDOPHENYL)-4-CYCLOPROPYL-N-(2-(TRIFLUOROMETHYL)PYRIDIN-4-YL)ISOTHIAZOLE-5-CARBOXAMIDE (I-148)

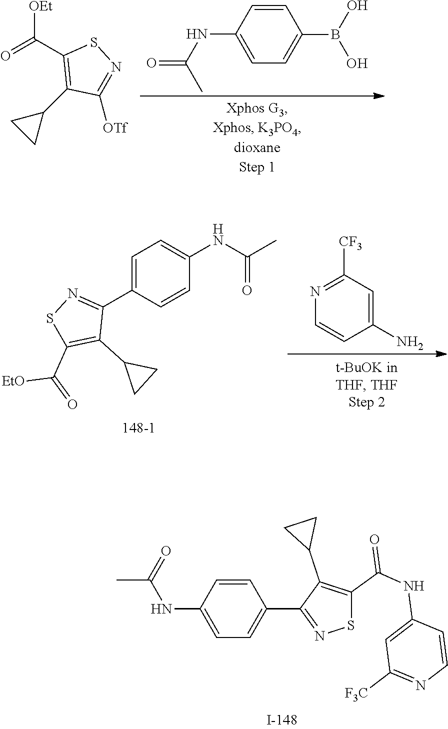

Step 1: Synthesis of 148-1: A solution of ethyl 4-cyclopropyl-3-(trifluoromethanesulfonyloxy)-1,2-thiazole-5-carboxylate (150 mg, 0.434 mmol, 1 equiv), 4-acetamidophenylboronic acid (93 mg, 0.520 mmol, 1.20 equiv), XPhos (41 mg, 0.086 mmol, 0.20 equiv), XPhos Pd G3 (73 mg, 0.086 mmol, 0.20 equiv) and K₃PO₄ (276 mg, 1.300 mmol, 2.99 equiv) in 1,4-dioxane (10 mL) was stirred overnight at 60° C. under a nitrogen atmosphere. The resulting mixture was diluted with water (10 mL). The resulting mixture was extracted with EtOAc (3×20 mL). The combined organic layers were washed with brine (1×40 mL) and dried over anhydrous Na₂SO₄. After filtration, the filtrate was concentrated under reduced pressure. The crude product (80 mg) was purified by prep-HPLC with the following conditions: (column: XBridge Prep OBD C18 column: 30*150 mm, 5 μm; mobile phase A: water (10 mmol/L NH₄HCO₃+0.1% NH₃·H₂O), mobile phase B: ACN; flow rate: 60 mL/min; gradient: 36% B to 65% B in 8 min, 65% B; wavelength: 220 nm; RT1(min): 7.47; injection volume: 1800 mL; number of runs: 1) to afford ethyl 4-cyclopropyl-3-(4-acetamidophenyl)-1,2-thiazole-5-carboxylate (148-1, 100 mg, 69.61%) as an off-white solid. LCMS (ES, m/z): [M+H]⁺=331

Step 2: Synthesis of 3-(4-acetamidophenyl)-4-cyclopropyl-N-(2-(trifluoromethyl)pyridin-4-yl)isothiazole-5-carboxamide (I-148): A solution of ethyl 4-cyclopropyl-3-(4-acetamidophenyl)-1,2-thiazole-5-carboxylate (80 mg, 0.242 mmol, 1 equiv), 2-(trifluoromethyl)pyridin-4-amine (47 mg, 0.290 mmol, 1.2 equiv) and t-BuOK (0.7 mL, 0.006 mmol, 0.03 equiv) in THF (5 mL) was stirred for 0.5 h at room temperature under a nitrogen atmosphere. The resulting mixture was diluted with water (5 mL). The resulting mixture was extracted with EtOAc (3×10 mL). The combined organic layers were washed with brine (1×20 mL) and dried over anhydrous Na₂SO₄. After filtration, the filtrate was concentrated under reduced pressure. The crude product (50 mg) was purified by prep-HPLC with the following conditions: (column: Xselect CSH C18 OBD column 30*150 mm 5 μm, n; mobile phase A: water (0.1% FA), mobile phase B: ACN; flow rate: 60 mL/min; gradient: 31% B to 41% B in 7 min, 41% B; wavelength: 254; 220 nm; RT1(min): 5.65, 8.32(min)) to afford 4-cyclopropyl-3-(4-acetamidophenyl)-N-[2-(trifluoromethyl)pyridin-4-yl]-1,2-thiazole-5-carboxamide (I-148, 28 mg, 25.88%) as an off-white solid. LCMS (ES, m/z): [M+H]±=447; ¹H NMR (400 MHz, DMSO-d₆) δ 0.19-0.42 (m, 2H), 0.71-0.99 (m, 2H), 2.09 (s, 3H), 2.15-2.28 (m, 1H), 7.73 (d, J=8.8 Hz, 2H), 7.81 (d, J=8.8 Hz, 2H), 7.90-8.05 (t, J=5.6 Hz, 1H), 8.22 (d, J=1.6 Hz, 1H), 8.72 (d, J=5.6 Hz, 1H), 10.14 (s, 1H), 11.31 (br s, 1H).

### EXAMPLE 236—SYNTHESIS OF 4-CYCLOPROPYL-3-(4-METHOXYPHENYL)-N-(2-(TRIFLUOROMETHYL)PYRIDIN-4-YL)ISOTHIAZOLE-5-CARBOXAMIDE (I-149)

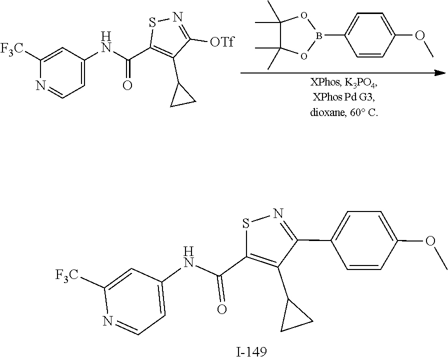

A mixture of 4-cyclopropyl-5-((2-(trifluoromethyl)pyridin-4-yl)carbamoyl)isothiazol-3-yl trifluoromethanesulfonate (100.00 mg, 0.217 mmol, 1 equiv), 2-(4-methoxyphenyl)-4,4,5,5-tetramethyl-1,3,2-dioxaborolane (60.89 mg, 0.260 mmol, 1.2 equiv), K₃PO₄ (138.03 mg, 0.651 mmol, 3 equiv), XPhos Pd G3 (36.69 mg, 0.043 mmol, 0.2 equiv) and XPhos (41.33 mg, 0.087 mmol, 0.4 equiv) in dioxane (2 mL) was stirred for 2 h at 60° C. under a nitrogen atmosphere. The resulting mixture was diluted with water (10 mL) and extracted with EtOAc (3×15 mL). The combined organic layers were washed with brine (1×40 mL) and dried over anhydrous Na₂SO₄. After filtration, the filtrate was concentrated under reduced pressure. The residue was purified by prep-HPLC with the following conditions: (column: XBridge Prep OBD C18 column: 30*150 mm, 5 μm; mobile phase A: water (10 mmol/L NH₄HCO₃+0.1% NH₃·H₂O), mobile phase B: ACN; flow rate: 60 mL/min; gradient: 51% B to 70% B in 8 min, 70% B; wavelength: 220 nm; RT1(min): 7.45; number of runs: 2) to afford 4-cyclopropyl-3-(4-methoxyphenyl)-N-(2-(trifluoromethyl)pyridin-4-yl)isothiazole-5-carboxamide (I-149, 4.0 mg, 4.39%) as an off-white solid. LCMS (ES, m/z): [M+H]⁺=420; ¹H NMR (400 MHz, DMSO-d₆) δ 0.26-0.38 (m, 2H), 0.77-0.91 (m, 2H), 2.16-2.28 (m, 1H), 3.84 (s, 3H), 7.08 (d, J=8.8 Hz, 2H), 7.82 (d, J=8.8 Hz, 2H), 7.94 (dd, J=2.0, 5.6 Hz, 1H), 8.22 (d, J=2.0 Hz, 1H), 8.72 (d, J=5.6 Hz, 1H), 11.30 (s, 1H).

### EXAMPLE 237—SYNTHESIS OF 4-CYCLOPROPYL-3-(1-METHYL-1H-PYRAZOL-3-YL)-N-(2-(TRIFLUOROMETHYL)PYRIDIN-4-YL)ISOTHIAZOLE-5-CARBOXAMIDE (I-150)

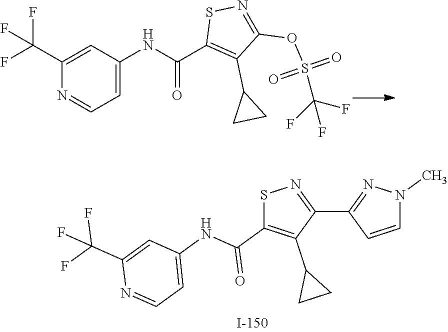 [SMILES: O=C(Nc1ccnc(C(F)(F)F)c1)c1snc(OS(=O)(=O)C(F)(F)F)c1C1CC1 ; Cn1ccc(-c2nsc(C(=O)Nc3ccnc(C(F)(F)F)c3)c2C2CC2)n1]

A solution of 4-cyclopropyl-5-{[2-(trifluoromethyl)pyridin-4-yl]carbamoyl}-1,2-thiazol-3-yl trifluoromethanesulfonate (100 mg, 0.217 mmol, 1 equiv) in toluene (2 mL) was treated with 1-methyl-3-(4,4,5,5-tetramethyl-1,3,2-dioxaborolan-2-yl)-1H-pyrazole (67.7 mg, 0.326 mmol, 1.5 equiv) for 5 min at room temperature under a nitrogen atmosphere followed by the addition of XPhos (20.7 mg, 0.043 mmol, 0.2 equiv), XPhos Pd G3 (73.4 mg, 0.087 mmol, 0.4 equiv) and K₃PO₄ (138 mg, 0.651 mmol, 3 equiv) at room temperature. The resulting mixture was stirred for an additional 2 h at 60° C. The residue was purified by reversed-phase flash chromatography with the following conditions: column: C18 silica gel; mobile phase: MeCN in water (0.1% TFA); 0% to 100% gradient in 20 min; detector: UV 254 nm. This resulted in 4-cyclopropyl-3-(1-methyl-1H-pyrazol-3-yl)-N-(2-(trifluoromethyl)pyridin-4-yl)isothiazole-5-carboxamide (1-150, 2.3 mg, 2.68%) as a white solid. LCMS (ES, m/z): [M+H]⁺=394; ¹H NMR (400 MHz, DMSO-d₆) δ 0.46 (d, J=5.6 Hz, 2H), 0.90 (d, J=8.8 Hz, 2H), 2.38-2.40 (m, 1H) 3.94 (s, 3H), 6.78 (d, J=2.4 Hz, 1H), 7.82 (d, J=2.0 Hz, 1H), 7.92 (d, J=6.0 Hz, 1H), 8.21 (s, 1H), 8.72 (d, J=5.2 Hz, 1H), 11.39 (s, 1H).

### EXAMPLE 238—SYNTHESIS OF 3-([1,2,4]TRIAZOLO[1,5-A]PYRIDIN-7-YL)-4-CYCLOPROPYL-N-(2-(TRIFLUOROMETHYL)PYRIDIN-4-YL)ISOTHIAZOLE-5-CARBOXAMIDE (I-151)

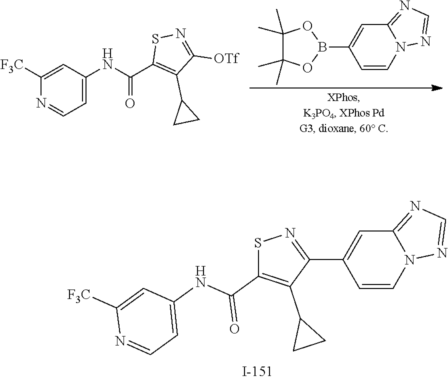 [SMILES: O=C(Nc1ccnc(C(F)(F)F)c1)c1snc(OS(=O)(=O)C(F)(F)F)c1C1CC1 ; CC1(C)OB(c2ccn3ncnc3c2)OC1(C)C ; O=C(Nc1ccnc(C(F)(F)F)c1)c1snc(-c2ccn3ncnc3c2)c1C1CC1]

A mixture of 4-cyclopropyl-5-{[2-(trifluoromethyl)pyridin-4-yl]carbamoyl}-1,2-thiazol-3-yl trifluoromethanesulfonate (100.00 mg, 0.217 mmol, 1 equiv), 7-(4,4,5,5-tetramethyl-1,3,2-dioxaborolan-2-yl)-[1,2,4]triazolo[1,5-a]pyridine (106.25 mg, 0.434 mmol, 2 equiv), XPhos Pd G3 (18.35 mg, 0.022 mmol, 0.1 equiv) and XPhos (20.67 mg, 0.043 mmol, 0.2 equiv) in dioxane (2 mL) was stirred for 2 h at 60° C. under a nitrogen atmosphere. The resulting mixture was diluted with water (10 mL) and extracted with EtOAc (3×15 mL). The combined organic layers were washed with brine (1×40 mL) and dried over anhydrous Na₂SO₄. After filtration, the filtrate was concentrated under reduced pressure. The crude product was purified by prep-HPLC with the following conditions: (column: XBridge Prep OBD C18 column: 30*150 mm, 5 μm; mobile phase A: water (10 mmol/L NH₄HCO₃), mobile phase B: ACN; flow rate: 60 mL/min; gradient: 30% B to 60% B in 8 min, 60% B, in 1 min; wavelength: 254 nm; RT1(min): 7.42; injection volume: 2 mL; number of runs: 1) to afford 3-([1,2,4]triazolo[1,5-a]pyridin-7-yl)-4-cyclopropyl-N-(2-(trifluoromethyl)pyridin-4-yl)isothiazole-5-carboxamide (I-151, 2.0 mg, 2.14%) as an off-white solid. LCMS (ES, m/z): [M+H]⁺=431; ¹H NMR (400 MHz, DMSO-d₆) δ 0.32-0.44 (m, 2H), 0.85-0.99 (m, 2H), 2.32-2.40 (m, 1H), 7.69 (dd, J=1.8, 7.2 Hz, 1H), 7.95 (dd, J=2.0, 5.4 Hz, 1H), 8.23 (d, J=2.0 Hz, 1H), 8.33 (d, J=1.4 Hz, 1H), 8.63 (s, 1H), 8.74 (d, J=5.6 Hz, 1H), 9.10 (d, J=5.6 Hz, 1H), 11.44 (s, 1H).

### EXAMPLE 239—SYNTHESIS OF 4-CYCLOPROPYL-3-(1-METHYL-1H-INDAZOL-4-YL)-N-(2-(TRIFLUOROMETHYL)PYRIDIN-4-YL)ISOTHIAZOLE-5-CARBOXAMIDE (I-152)

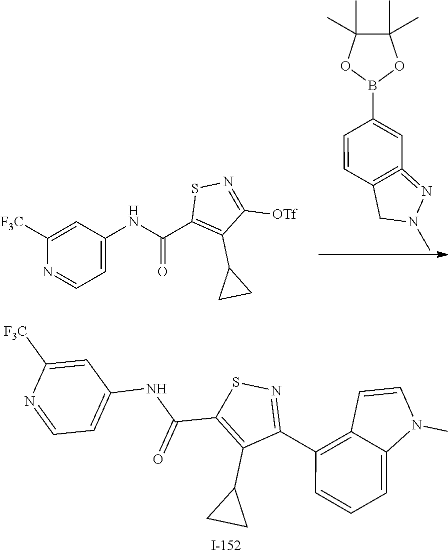

A mixture of 4-cyclopropyl-5-((2-(trifluoromethyl)pyridin-4-yl)carbamoyl)isothiazol-3-yl trifluoromethanesulfonate (100.00 mg, 0.217 mmol, 1 equiv), 1-methyl-4-(4,4,5,5-tetramethyl-1,3,2-dioxaborolan-2-yl)-1H-indazole (72.74 mg, 0.282 mmol, 1.3 equiv), X-Phos (25.83 mg, 0.054 mmol, 0.25 equiv), XPhos Pd G3 (36.69 mg, 0.043 mmol, 0.2 equiv) and K₃PO₄ (138.03 mg, 0.651 mmol, 3 equiv) in dioxane (3 mL) was stirred for 2 h at 60° C. under a nitrogen atmosphere. The resulting mixture was diluted with water (10 mL) and extracted with EtOAc (3×15 mL). The combined organic layers were washed with brine (1×40 mL) and dried over anhydrous Na₂SO₄. After filtration, the filtrate was concentrated under reduced pressure. The crude product was purified by prep-HPLC with the following conditions: column: XBridge Prep OBD C18 column: 30*150 mm, 5 μm; mobile phase A: water (10 mmol/L NH₄HCO₃+0.1% NH₃·H₂O), mobile phase B: ACN; flow rate: 60 mL/min; gradient: 39% B to 69% B in 8 min, 69% B; wavelength: 220 nm; RT1(min): 7.57, to afford 4-cyclopropyl-3-(1-methyl-1H-indazol-4-yl)-N-(2-(trifluoromethyl)pyridin-4-yl)isothiazole-5-carboxamide (1-152, 20.8 mg, 21.62%) as an off-white solid. LCMS (ES, m/z): [M+H]^{m}=444; ¹H NMR (400 MHz, DMSO-d₆) δ 0.21-0.35 (m, 2H), 0.70-0.85 (m, 2H), 2.18-2.30 (m, 1H), 4.12 (s, 3H), 7.56 (t, J=8.0 Hz, 1H), 7.63 (d, J=8.0 Hz, 1H), 7.80 (d, J=8.0 Hz, 1H), 7.96 (d, J=8.0 Hz, 1H), 8.20 (s, 1H), 8.24 (d, J=2.0 Hz, 1H), 8.74 (d, J=5.5 Hz, 1H), 11.36 (s, 1H).

### EXAMPLE 240—SYNTHESIS OF 3-(BENZO[D]THIAZOL-6-YL)-4-CYCLOPROPYL-N-(2-(TRIFLUOROMETHYL)PYRIDIN-4-YL)ISOTHIAZOLE-5-CARBOXAMIDE (I-153)

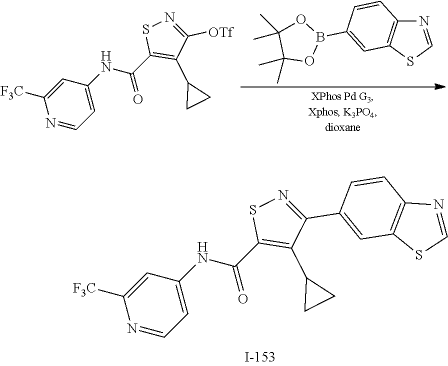 [SMILES: O=C(Nc1ccnc(C(F)(F)F)c1)c1snc(OS(=O)(=O)C(F)(F)F)c1C1CC1 ; CC1(C)OB(c2ccc3ncsc3c2)OC1(C)C ; O=C(Nc1ccnc(C(F)(F)F)c1)c1snc(-c2ccc3ncsc3c2)c1C1CC1]

A solution of 4-cyclopropyl-5-{[2-(trifluoromethyl)pyridin-4-yl]carbamoyl}-1,2-thiazol-3-yl trifluoromethanesulfonate (100 mg, 0.217 mmol, 1 equiv), 6-(4,4,5,5-tetramethyl-1,3,2-dioxaborolan-2-yl)-1,3-benzothiazole (84.91 mg, 0.326 mmol, 1.5 equiv), K₃PO₄ (138.03 mg, 0.651 mmol, 3 equiv), XPhos (10.33 mg, 0.022 mmol, 0.1 equiv) and XPhos Pd G3 (18.35 mg, 0.022 mmol, 0.1 equiv) in dioxane (10 mL) was stirred overnight at 60° C. under a nitrogen atmosphere. The reaction was quenched with water at room temperature. The resulting mixture was extracted with EtOAc (2×20 mL). The combined organic layers were washed with brine (1×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 40% to 50% gradient in 10 min; detector: UV 254 nm. This resulted in 3-(1,3-benzothiazol-6-yl)-4-cyclopropyl-N-[2-(trifluoromethyl)pyridin-4-yl]-1,2-thiazole-5-carboxamide (I-153, 5.8 mg, 5.97%) as an off-white solid. LCMS (ES, m/z): [M+H]⁺=447; ¹H NMR (400 MHz, DMSO-d₆): δ 0.28-0.36 (m, 2H), 0.80-0.90 (m, 2H), 2.28-2.32 (m, 1H), 7.94 (dd, J=5.6, 2.0 Hz, 1H), 8.04 (dd, J=8.6, 1.8 Hz, 1H), 8.20-8.26 (m, 2H), 8.66-8.76 (m, 2H), 9.50 (s, 1H), 11.16 (s, 1H).

### EXAMPLE 241—SYNTHESIS OF 4-CYCLOPROPYL-3-(4-(DIFLUOROMETHYL) PHENYL)-N-(2-(TRIFLUOROMETHYL)PYRIDIN-4-YL)ISOTHIAZOLE-5-CARBOXAMIDE (I-154)

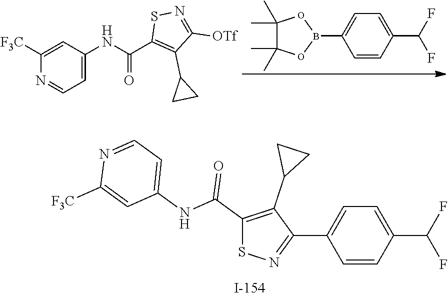

A mixture of 4-cyclopropyl-5-((2-(trifluoromethyl)pyridin-4-yl)carbamoyl)isothiazol-3-yl trifluoromethanesulfonate (90.00 mg, 0.195 mmol, 1 equiv). 2-(4-(difluoromethyl)phenyl)-4,4,5,5-tetramethyl-1,3,2-dioxaborolane (59.48 mg, 0.234 mmol, 1.2 equiv), XPhos Pd G3 (33.03 mg, 0.039 mmol, 0.2 equiv), X-Phos (23.25 mg, 0.049 mmol, 0.25 equiv) and K₃PO₄ (124.23 mg, 0.585 mmol, 3 equiv) in dioxane (3 mL) was stirred for 2 h at 60° C. under a nitrogen atmosphere. The resulting mixture was diluted with water (10 mL) and extracted with EtOAc (3×15 mL). The combined organic layers were washed with brine (1×40 mL) and dried over anhydrous Na₂SO₄. After filtration, the filtrate was concentrated under reduced pressure. The crude product was purified by prep-HPLC with the following conditions: column: XBridge Prep Phenyl OBD column: 19*250 mm, 5 μm; mobile phase A: water (10 mmol/L NH₄HCO₃+0.1% NH₃·H₂O), mobile phase B: ACN; flow rate: 25 mL/min; gradient: 52% B to 68% B in 11 min, 68% B; wavelength: 220/254 nm; RT1(min): 10.35; injection volume: 0.5 mL, to afford 4-cyclopropyl-3-(4-(difluoromethyl)phenyl)-N-(2-(trifluoromethyl)pyridin-4-yl)isothiazole-5-carboxamide (1-154, 27.70 mg, 32.12%) as an off-white solid. LCMS (ES, m/z): [M+H]⁺=440; ¹H NMR (400 MHz, DMSO-d₆) δ 0.24-0.40 (m, 2H), 0.78-0.92 (m, 2H), 2.18-2.30 (m, 1H), 7.15 (t, J=56.0 Hz, 1H), 7.74 (d, J=8.0 Hz, 2H), 7.97 (d, J=5.2 Hz, 1H), 8.01 (d, J=8.0 Hz, 2H), 8.23 (d, J=2.0 Hz, 1H), 8.73 (d, J=8.0 Hz, 1H), 11.37 (s, 1H).

### EXAMPLE 242—SYNTHESIS OF 4-CYCLOPROPYL-3-(3-FLUOROPYRIDIN-4-YL)-N-(2-(TRIFLUOROMETHYL)PYRIDIN-4-YL)ISOTHIAZOLE-5-CARBOXAMIDE (I-155)

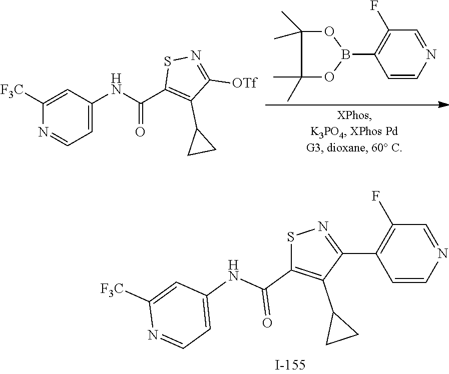

To a stirred solution of 4-cyclopropyl-5-{[2-(trifluoromethyl)pyridin-4-yl]carbamoyl}-1,2-thiazol-3-yl trifluoromethanesulfonate (90 mg, 0.195 mmol, 1 equiv) and 3-fluoro-4-(4,4,5,5-tetramethyl-1,3,2-dioxaborolan-2-yl)pyridine (52.22 mg, 0.234 mmol, 1.2 equiv) in 1,4-dioxane (2 mL) was added XPhos (9.30 mg, 0.020 mmol, 0.1 equiv), XPhos Pd G3 (16.51 mg, 0.020 mmol, 0.1 equiv) and K₃PO₄ (103.52 mg, 0.488 mmol, 2.5 equiv) in portions at room temperature under a nitrogen atmosphere. The resulting mixture was stirred for an additional 2 h at 60° C. The resulting mixture was diluted with water (20 mL). The resulting mixture was extracted with EtOAc (3×30 mL). The combined organic layers were washed with brine (1×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 50% to 60% gradient in 10 min; detector: UV 254 nm. The resulting mixture was concentrated under reduced pressure. This resulted in 4-cyclopropyl-3-(3-fluoropyridin-4-yl)-N-[2-(trifluoromethyl)pyridin-4-yl]-1,2-thiazole-5-carboxamide (I-155, 11.2 mg, 13.99%) as a white solid. LCMS (ES, m/z): [M+H]⁺=409; ¹H NMR (400 MHz, DMSO-d₆) δ 0.28 (d, J=5.6 Hz, 2H), 0.72 (d, J=8.0 Hz, 2H), 2.07-2.13 (m, 1H), 7.67 (m, 1H), 7.96 (m, 1H), 8.23 (s, 1H), 8.62 (d, J=4.6 Hz, 1H), 8.73 (d, J=5.6 Hz, 1H), 8.81 (s, 1H), 11.41 (s, 1H).

### EXAMPLE 243—SYNTHESIS OF 4-CYCLOPROPYL-3-(3-METHOXYPHENYL)-N-(2-(TRIFLUOROMETHYL)PYRIDIN-4-YL)ISOTHIAZOLE-5-CARBOXAMIDE (I-156)

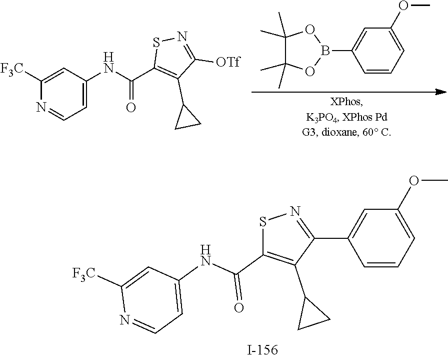

A mixture of 4-cyclopropyl-5-((2-(trifluoromethyl)pyridin-4-yl)carbamoyl)isothiazol-3-yl trifluoromethanesulfonate (100.00 mg, 0.217 mmol, 1 equiv), 2-(3-methoxyphenyl)-4,4,5,5-tetramethyl-1,3,2-dioxaborolane (60.89 mg, 0.260 mmol, 1.2 equiv), X-Phos (25.83 mg, 0.054 mmol, 0.25 equiv), XPhos Pd G3 (36.69 mg, 0.043 mmol, 0.2 equiv) and K₃PO₄ (138.03 mg, 0.651 mmol, 3 equiv) in dioxane (3 mL) was stirred for 2 h at 60° C. under a nitrogen atmosphere. The resulting mixture was diluted with water (10 mL) and extracted with EtOAc (3×15 mL). The combined organic layers were washed with brine (1×40 mL) and dried over anhydrous Na₂SO₄. After filtration, the filtrate was concentrated under reduced pressure. The crude product was purified by prep-HPLC with the following conditions: column: XBridge Shield RP18 OBD column: 30*150 mm, 5 μm; mobile phase A: water (10 mmol/L NH₄HCO₃+0.1% NH₃·H₂O), mobile phase B: ACN; flow rate: 60 mL/min; gradient: 48% B to 70% B in 8 min, 70% B; wavelength: 220/254 nm; RT1(min): 7.48; injection volume: 1.5 mL, to afford 4-cyclopropyl-3-(3-methoxyphenyl)-N-(2-(trifluoromethyl)pyridin-4-yl)isothiazole-5-carboxamide (I-156, 23.5 mg, 25.77%) as an off-white solid. LCMS (ES, m/z): [M+H]⁺=420; ¹H NMR (400 MHz, DMSO-d₆) δ 0.27-0.38 (m, 2H), 0.78-0.89 (m, 2H), 2.14-2.27 (m, 1H), 3.83 (s, 3H), 7.08 (d, J=7.6 Hz, 1H), 7.36 (s, 1H), 7.39-7.50 (m, 2H), 7.93 (d, J=4.0 Hz, 1H), 8.22 (d, J=2.0 Hz, 1H), 8.72 (d, J=4.0 Hz, 1H), 11.35 (s, 1H).

### EXAMPLE 244—SYNTHESIS OF 4-CYCLOPROPYL-3-(2-METHYL-2H-INDAZOL-4-YL)-N-(2-(TRIFLUOROMETHYL)PYRIDIN-4-YL)ISOTHIAZOLE-5-CARBOXAMIDE (I-157)

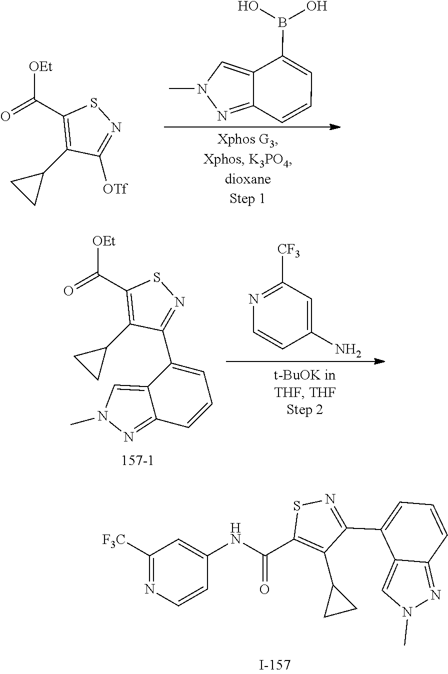 [SMILES: CCOC(=O)c1snc(OS(=O)(=O)C(F)(F)F)c1C1CC1 ; Cn1cc2c(B(O)O)cccc2n1 ; CCOC(=O)c1snc(-c2cccc3nn(C)cc23)c1C1CC1 ; Nc1ccnc(C(F)(F)F)c1 ; Cn1cc2c(-c3nsc(C(=O)Nc4ccnc(C(F)(F)F)c4)c3C3CC3)cccc2n1]

Step 1: Synthesis of 157-1: A solution of ethyl 4-cyclopropyl-3-(trifluoromethanesulfonyloxy)-1,2-thiazole-5-carboxylate (150 mg, 0.434 mmol, 1 equiv), 2-methylindazol-4-ylboronic acid (92 mg, 0.523 nmol, 1.20 equiv), XPhos (42 mg, 0.088 mmol, 0.20 equiv), XPhos Pd G3 (74 mg, 0.087 mmol, 0.20 equiv) and K₃PO₄ (276 mg, 1.300 mmol, 2.99 equiv) in 1,4-dioxane (10 mL) was stirred overnight at 60° C. under a nitrogen atmosphere. The resulting mixture was diluted with water (10 mL). The resulting mixture was extracted with EtOAc (3×20 mL). The combined organic layers were washed with brine (1×20 mL) and dried over anhydrous Na₂SO₄. After filtration, the filtrate was concentrated under reduced pressure. The crude product (80 mg) was purified by prep-HPLC with the following conditions: (column: XBridge Prep OBD C18 column: 30*150 mm, 5 μm; mobile phase A: water (10 mmol/L NH₄HCO₃+0.1% NH₃·H₂O), mobile phase B: ACN; flow rate: 60 mL/min; gradient: 36% B to 65% B in 8 min, 65% B; wavelength: 220 nm; RT1(min): 7.47; injection volume: 1800 mL; number of runs: 1) to afford ethyl 4-cyclopropyl-3-(2-methylindazol-4-yl)-1,2-thiazole-5-carboxylate (157-1, 100 mg, 70.31%) as a light yellow oil. LCMS (ES, m/z): [M+H]⁺=328.

Step 2: Synthesis of 4-cyclopropyl-3-(2-methyl-2H-indazol-4-yl)-N-(2-(trifluoromethyl)pyridin-4-yl)isothiazole-5-carboxamide (I-157): A solution of ethyl 4-cyclopropyl-3-(2-methylindazol-4-yl)-1,2-thiazole-5-carboxylate (100 mg, 0.305 mmol, 1 equiv), 2-(trifluoromethyl)pyridin-4-amine (60 mg, 0.370 mmol, 1.21 equiv) and t-BuOK (100 mg, 0.891 mmol, 2.92 equiv) in THF (10 mL) was stirred for 0.5 h at room temperature under a nitrogen atmosphere. The resulting mixture was diluted with water (5 mL). The resulting mixture was extracted with EtOAc (3×10 mL). The combined organic layers were washed with brine (1×20 mL) and dried over anhydrous Na₂SO₄. After filtration, the filtrate was concentrated under reduced pressure. The crude product (50 mg) was purified by prep-HPLC with the following conditions: (column: Xselect CSH C18 OBD column 30*150 mm 5 μm, n; mobile phase A: water (0.1% FA), mobile phase B: ACN; flow rate: 60 mL/min; gradient: 31% B to 41% B in 7 min, 41% B; wavelength: 254; 220 nm; RT1(min): 5.65, 8.32(min)) to afford 4-cyclopropyl-3-(2-methylindazol-4-yl)-N-[2-(trifluoromethyl)pyridin-4-yl]-1,2-thiazole-5-carboxamide (I-157, 4.1 mg, 3.02%) as an off-white solid. LCMS (ES, m/z): [M+H]⁺=444; ¹H NMR (400 MHz, DMSO-d₆) δ 0.21-0.44 (m, 2H), 0.71-0.99 (m, 2H), 2.21-2.31 (m, 1H), 4.21 (s, 3H), 7.40 (dd, J=8.4, 6.8 Hz, 1H), 7.66 (d, J=6.4 Hz, 1H), 7.74 (d, J=8.8 Hz, 1H), 7.92 (d, J=2.0 Hz, 1H), 8.24 (d, J=2.0 Hz, 1H), 8.55 (s, 1H), 8.74 (d, 0.1=2.8 Hz, 1H), 11.41 (s, 1H).

### EXAMPLE 245—SYNTHESIS OF 3-(3-CYANOPHENYL)-4-CYCLOPROPYL-N-(2-(TRIFLUOROMETHYL)PYRIDIN-4-YL)ISOTHIAZOLE-5-CARBOXAMIDE (I-158)

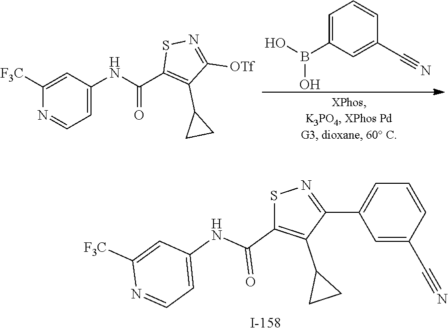 [SMILES: O=C(Nc1ccnc(C(F)(F)F)c1)c1snc(OS(=O)(=O)C(F)(F)F)c1C1CC1 ; N#Cc1cccc(B(O)O)c1 ; N#Cc1cccc(-c2nsc(C(=O)Nc3ccnc(C(F)(F)F)c3)c2C2CC2)c1]

A solution of 4-cyclopropyl-5-{[2-(trifluoromethyl)pyridin-4-yl]carbamoyl}-1,2-thiazol-3-yl trifluoromethanesulfonate (80 mg, 0.173 mmol, 1 equiv), 3-cyanophenylboronic acid (50.96 mg, 0.346 mmol, 2 equiv), XPhos Pd G3 (14.68 mg, 0.017 mmol, 0.1 equiv), XPhos (8.27 mg, 0.017 mmol, 0.1 equiv) and K₃PO₄ (73.62 mg, 0.346 mmol, 2 equiv) in dioxane (2 mL) was stirred for 12 h at 60° C. under a nitrogen atmosphere. The resulting mixture was diluted with water (30 mL). The resulting mixture was extracted with EtOAc (3×50 mL). The combined organic layers were washed with brine (1×10 mL) and dried over anhydrous Na₂SO₄. After filtration, the filtrate was concentrated under reduced pressure. The residue was purified by reversed-phase flash chromatography with the following conditions: column: Xselect CSH C18 OBD column 30*150 mm 5 μm, n; mobile phase A: water (0.1% FA), mobile phase B: ACN; flow rate: 60 mL/min; gradient: 47% B to 70% B in 7 min, 70% B; wavelength: 220 nm; RT1(min): 6.5; number of runs: 2; to afford 3-(3-cyanophenyl)-4-cyclopropyl-N-[2-(trifluoromethyl)pyridin-4-yl]-1,2-thiazole-5-carboxamide (I-158, 27.1 mg, 37.52%) as a white solid. LCMS (ES, m/z): [M+H]⁺=415; ¹H NMR (400 MHz, DMSO-d₆) δ 0.26-0.35 (m, 2H), 0.81-0.90 (m, 2H), 2.27 (tt, J=8.6, 5.6 Hz, 1H), 7.77 (t, J=7.8 Hz, 1H), 7.94 (dd, J=5.6, 2.0 Hz, 1H), 8.00 (m, J=7.8, 1.4 Hz, 1H), 8.16-8.24 (m, 2H), 8.26 (d, J=1.8 Hz, 1H), 8.73 (d, J=5.6 Hz, 1H), 11.41 (s, 1H).

### EXAMPLE 246—SYNTHESIS OF 3-(BENZO[D]THIAZOL-4-YL)-4-CYCLOPROPYL-N-(3-(TRIFLUOROMETHYL)PHENYL)ISOTHIAZOLE-5-CARBOXAMIDE (I-159)

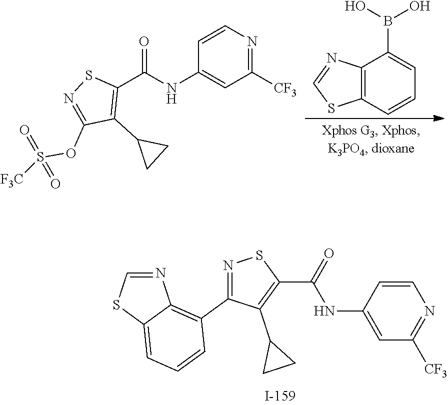

A solution of 4-cyclopropyl-5-{[2-(trifluoromethyl)pyridin-4-yl]carbamoyl}-1,2-thiazol-3-yl trifluoromethanesulfonate (150 mg, 0.325 mmol, 1 equiv), 1,3-benzothiazol-4-ylboronic acid (64 mg, 0.358 mmol, 1.10 equiv), XPhos (31.00 mg, 0.065 mmol, 0.2 equiv), XPhos Pd G₃ (55 mg, 0.065 mmol, 0.20 equiv) and K₃PO₄ (207 mg, 0.975 mmol, 3.00 equiv) in 1,4-dioxane (10 mL) was stirred overnight at 60° C. under a nitrogen atmosphere. The resulting mixture was diluted with water (10 mL). The resulting mixture was extracted with EtOAc (3×20 mL). The combined organic layers were washed with brine (1×40 mL) and dried over anhydrous Na₂SO₄. After filtration, the filtrate was concentrated under reduced pressure. The crude product (80 mg) was purified by prep-HPLC with the following conditions: (column: XBridge Prep OBD C18 column: 30*150 mm, 5 μm; mobile phase A: water (10 mmol/L NH₄HCO₃+0.1% NH₃·H₂O), mobile phase B: ACN; flow rate: 60 mL/min; gradient: 36% B to 65% B in 8 min, 65% B; wavelength: 220 nm; RT1(min): 7.47; number of runs: 1) to afford 3-(1,3-benzothiazol-4-yl)-4-cyclopropyl-N-[2-(trifluoromethyl)pyridin-4-yl]-1,2-thiazole-5-carboxamide (I-159, 17 mg, 11.68%) as an off-white solid. LCMS (ES, m % z): [M+H]⁺=447; ¹H NMR (400 MHz, DMSO-d₆) δ 0.11-0.31 (m, 2H), 0.30-0.57 (m, 2H), 1.97-2.25 (m, 1H), 7.48-7.78 (m, 2H), 8.00 (dd, J=5.6, 2.0 Hz, 1H), 8.26 (d, J=2.0 Hz, 1H), 8.35 (dd, J=6.4, 2.8 Hz, 1H), 8.72 (d, J=5.2 Hz, 1H), 9.45 (s, 1H), 11.43 (s, 1H).

### EXAMPLE 247—SYNTHESIS OF 4-CYCLOPROPYL-3-(O-TOLYL)-N-(2-(TRIFLUOROMETHYL)PYRIDIN-4-YL)ISOTHIAZOLE-5-CARBOXAMIDE (I-160)

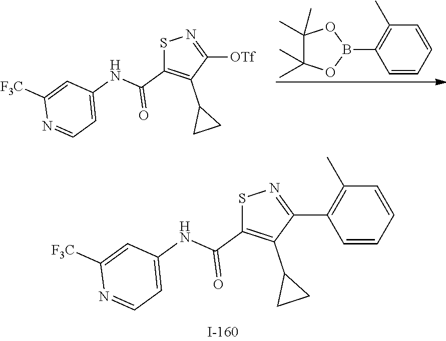 [SMILES: O=C(Nc1ccnc(C(F)(F)F)c1)c1snc(OS(=O)(=O)C(F)(F)F)c1C1CC1 ; Cc1ccccc1B1OC(C)(C)C(C)(C)O1 ; Cc1ccccc1-c1nsc(C(=O)Nc2ccnc(C(F)(F)F)c2)c1C1CC1]

A solution of 4-cyclopropyl-5-{[2-(trifluoromethyl)pyridin-4-yl]carbamoyl}-1,2-thiazol-3-yl trifluoromethanesulfonate (100 mg, 0.217 mmol, 1 equiv), 4,4,5,5-tetramethyl-2-(o-tolyl)-1,3,2-dioxaborolane (56 mg, 0.260 mmol, 1.2 equiv), XPhos Pd G3 (36 mg, 0.043 mmol, 0.2 equiv), XPhos (25 mg, 0.054 mmol, 0.25 equiv) and K₃PO₄ (138.03 mg, 0.651 mmol, 3 equiv) in dioxane (2 mL) was stirred for 2 h at 60° C. under a nitrogen atmosphere. The mixture was allowed to cool down to room temperature. The resulting mixture was diluted with water (10 mL). The resulting mixture was extracted with EtOAc (3×10 mL). The combined organic layers were washed with water (1×30 mL) and dried over anhydrous Na₂SO₄. After filtration, the filtrate was concentrated under reduced pressure. The crude product was purified by prep-HPLC with the following conditions: column: XBridge Prep OBD C18 column: 30*150 mm, 5 m; mobile phase A: water (10 mmol/L NH₄HCO₃), mobile phase B: ACN; flow rate: 60 mL/min; gradient: 55% B to 75% B in 8 min, 75% B; wavelength: 254 nm; RT1(min): 7.27; to afford 4-cyclopropyl-3-(2-methylphenyl)-N-[2-(trifluoromethyl)pyridin-4-yl]-1,2-thiazole-5-carboxamide (I-160, 2.2 mg, 2.46%) as an off-white solid. LCMS (ES, m/z): [M+H]⁺=404; ¹H NMR (400 MHz, DMSO-d₆): δ 0.24-0.41 (m, 2H), 0.64-072 (m, 2H), 1.93-2.01 (m, 1H), 2.20 (s, 3H), 7.30-7.50 (m, 4H), 7.95 (s, 1H), 8.21 (s, 1H), 8.72 (s, 1H), 11.33 (s, 1H).

### EXAMPLE 248—SYNTHESIS OF 4-CYCLOPROPYL-3-(2-METHOXYPHENYL)-N-(2-(TRIFLUOROMETHYL)PYRIDIN-4-YL)ISOTHIAZOLE-5-CARBOXAMIDE (I-161)

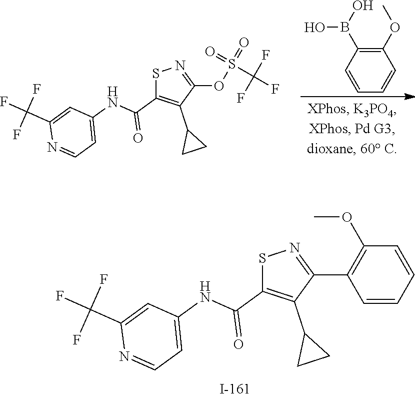 [SMILES: O=C(Nc1ccnc(C(F)(F)F)c1)c1snc(OS(=O)(=O)C(F)(F)F)c1C1CC1 ; COc1ccccc1B(O)O ; COc1ccccc1-c1nsc(C(=O)Nc2ccnc(C(F)(F)F)c2)c1C1CC1]

A solution of 4-cyclopropyl-5-{[2-(trifluoromethyl)pyridin-4-yl]carbamoyl}-1,2-thiazol-3-yl trifluoromethanesulfonate (80 mg, 0.173 mmol, 1 equiv), 2-methoxyphenylboronic acid (52.70 mg, 0.346 mmol, 2 equiv), XPhos (8.27 mg, 0.017 mmol, 0.1 equiv), XPhos Pd G3 (14.68 mg, 0.017 mmol, 0.1 equiv) and K₃PO₄ (73.62 mg, 0.346 mmol, 2 equiv) in dioxane (5 mL) was stirred for 12 h at 60° C. under a nitrogen atmosphere. The resulting mixture was diluted with water (20 mL). The resulting mixture was extracted with EtOAc (3×50 mL). The combined organic layers were washed with brine (1×10 mL) and dried over anhydrous Na₂SO₄. After filtration, the filtrate was concentrated under reduced pressure. The crude product (30 mg) was purified by prep-HPLC with the following conditions: (column: Xselect CSH C18 OBD column 30*150 mm 5 m; mobile phase A: water (0.1% FA), mobile phase B: ACN; flow rate: 60 mL/min; gradient: 47% B to 73% B in 7 min, 73% B; wavelength: 220 nm; RT1(min): 6.60; number of runs: 2) to afford 4-cyclopropyl-3-(2-methoxyphenyl)-N-[2-(trifluoromethyl)pyridin-4-yl]-1,2-thiazole-5-carboxamide (1-161, 8.8 mg, 12.09%) as a white solid. LCMS (ES, m/z): [M+H]⁺=420; ¹H NMR (400 MHz, DMSO-d₆) δ 0.23-0.24 (m, 2H), 0.58-0.61 (m, 2H), 1.96 (tt, J=8.6, 5.6 Hz, 1H), 3.81 (s, 3H), 7.06 (m, J=7.4, 1.0 Hz, 1H), 7.17 (d, J=8.4 Hz, 1H), 7.28 (dd, J=7.6, 1.8 Hz, 1H), 7.47-7.51 (m, 1H), 7.96 (dd, J=5.4, 2.0 Hz, 1H), 8.22 (d, J=2.0 Hz, 1H), 8.71 (d, J=5.6 Hz, 1H), 11.35 (s, 1H).

### EXAMPLE 249—SYNTHESIS OF 3-(BENZO[D]OXAZOL-4-YL)-4-CYCLOPROPYL-N-(2-(TRIFLUOROMETHYL)PYRIDIN-4-YL)ISOTHIAZOLE-5-CARBOXAMIDE (I-162

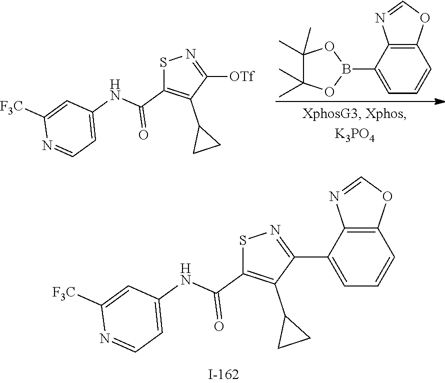 [SMILES: O=C(Nc1ccnc(C(F)(F)F)c1)c1snc(OS(=O)(=O)C(F)(F)F)c1C1CC1 ; CC1(C)OB(c2cccc3ocnc23)OC1(C)C ; O=C(Nc1ccnc(C(F)(F)F)c1)c1snc(-c2cccc3ocnc23)c1C1CC1]

A solution of 4-cyclopropyl-5-{[2-(trifluoromethyl)pyridin-4-yl]carbamoyl}-1,2-thiazol-3-yl trifluoromethanesulfonate (100 mg, 0.217 mmol, 1 equiv), 4-(4,4,5,5-tetramethyl-1,3,2-dioxaborolan-2-yl)-1,3-benzoxazole (79 mg, 0.326 mmol, 1.5 equiv), XPhos Pd G3 (36 mg, 0.043 mmol, 0.2 equiv), XPhos (25 mg, 0.054 mmol, 0.25 equiv) and K₃PO₄ (138 mg, 0.651 mmol, 3 equiv) in dioxane (2 mL) was stirred for 2 h at 60° C. under a nitrogen atmosphere. The mixture was allowed to cool down to room temperature. The resulting mixture was diluted with water (10 mL). The resulting mixture was extracted with EtOAc (3×10 mL). The combined organic layers were washed with water (1×30 mL) and dried over anhydrous Na₂SO₄. After filtration, the filtrate was concentrated under reduced pressure. The crude product was purified by prep-HPLC with the following conditions: column: XBridge Prep OBD C18 column: 30*150 mm, 5 μm; mobile phase A: water (10 mmol/L NH₄HCO₃+0.1% NH₃·H₂O), mobile phase B: ACN; flow rate: 60 mL/min; gradient: 40% B to 67% B in 8 min, 67% B; wavelength: 220 nm; RT1(min): 7.57 to afford 3-(1,3-benzoxazol-4-yl)-4-cyclopropyl-N-[2-(trifluoromethyl)pyridin-4-yl]-1,2-thiazole-5-carboxamide (I-162, 22.6 mg, 23.86%) as an off-white solid. LCMS (ES, m/z): [M+H]⁺=431; ¹H NMR (400 MHz, DMSO-d₆) δ 015-0.23 (m, 2H), 0.42-0.59 (m, 2H), 2.25-2.36 (m, 1H), 7.55 (s, 1H), 7.59 (d, J=7.6 Hz, 1H), 7.92-8.02 (m, 2H), 8.25 (d, J=1.2 Hz, 1H), 8.72 (d, J=5.2 Hz, 1H), 8.85 (s, 1H), 11.39 (s, 1H).

### EXAMPLE 250—SYNTHESIS OF 4-CYCLOPROPYL-3-(2-METHOXYPYRIDIN-3-YL)-N-(2-(TRIFLUOROMETHYL)PYRIDIN-4-YL)ISOTHIAZOLE-5-CARBOXAMIDE (I-163)

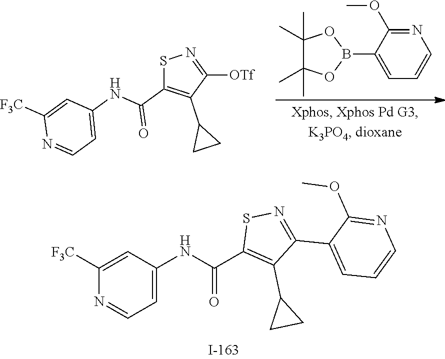 [SMILES: O=C(Nc1ccnc(C(F)(F)F)c1)c1snc(OS(=O)(=O)C(F)(F)F)c1C1CC1 ; COc1ncccc1B1OC(C)(C)C(C)(C)O1 ; COc1ncccc1-c1nsc(C(=O)Nc2ccnc(C(F)(F)F)c2)c1C1CC1]

To a stirred solution of 4-cyclopropyl-5-{[2-(trifluoromethyl)pyridin-4-yl]carbamoyl}-1,2-thiazol-3-yl trifluoromethanesulfonate (100 mg, 0.217 mmol, 1 equiv), XPhos (41.3 mg, 0.087 mmol, 0.40 equiv), XPhos Pd G3 (18.4 mg, 0.022 mmol, 0.10 equiv) and K₃PO₄ (138 mg, 0.650 mmol, 3.00 equiv) in dioxane (5 mL) was added 2-methoxy-3-(4,4,5,5-tetramethyl-1,3,2-dioxaborolan-2-yl)pyridine (66.2 mg, 0.282 mmol, 1.30 equiv) in portions at room temperature under a nitrogen atmosphere. The resulting mixture was stirred for 3 h at 60° C. under a nitrogen atmosphere. The resulting mixture was filtered, and the filter cake was washed with 1,4-dioxane (3×5 mL). The filtrate was concentrated under reduced pressure. The crude product (100 mg) was purified by prep-HPLC with the following conditions: (column: Xselect CSH C18 OBD column 30*150 mm 5 μm; mobile phase A: water (0.1% FA), mobile phase B: ACN; flow rate: 60 mL/min; gradient: 50% B to 60% B in 7 min, 60% B; wavelength: 254; 220 nm; RT1(min): 6.55; number of runs: 1) to afford 4-cyclopropyl-3-(2-methoxypyridin-3-yl)-N-(2-(trifluoromethyl)pyridin-4-yl)isothiazole-5-carboxamide (I-163, 9.4 mg, 10.31%) as a white solid. LCMS (ES, m/z): [M+1]⁺=421; ¹H NMR (400 MHz, DMSO-d₆) δ 0.21-0.25 (m, 2H), 0.54-0.68 (m, 2H), 2.02 (s, 1H), 3.92 (s, 3H), 7.15 (dd, J=5.2, 1.2 Hz, 1H), 7.78 (dd, J=7.6, 2.0 Hz, 1H), 7.96 (d, J=5.2 Hz, 1H), 8.22 (s, 1H), 8.33 (dd, J=4.8, 2.0 Hz, 1H), 8.71 (d, J=5.2 Hz, 1H), 11.36 (s, 1H).

### EXAMPLE 251—SYNTHESIS OF 4-CYCLOPROPYL-3-(2-(TRIFLUOROMETHYL)PHENYL)-N-(2-(TRIFLUOROMETHYL)PYRIDIN-4-YL)ISOTHIAZOLE-5-CARBOXAMIDE (I-164)

Step 1: Synthesis of 164-1: To a stirred solution of ethyl 4-cyclopropyl-3-(trifluoromethanesulfonyloxy)-1,2-thiazole-5-carboxylate (150 mg, 0.434 mmol, 1 equiv), K₃PO₄ (276.62 mg, 1.302 mmol, 3 equiv) and 2-(trifluoromethyl)phenylboronic acid (123.76 mg, 0.651 mmol, 1.5 equiv) in dioxane (3 mL) was added XPhos (20.71 mg, 0.043 mmol, 0.1 equiv) and XPhos Pd G3 (36.77 mg, 0.043 mmol, 0.1 equiv) in portions. The resulting mixture was stirred for 2 h at 60° C. under a nitrogen atmosphere. The reaction was quenched with water at room temperature. The resulting mixture was extracted with EtOAc (3×50 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.10% TFA); 10% to 50% gradient in 10 min; detector: UV 254 nm. This resulted in ethyl 4-cyclopropyl-3-[2-(trifluoromethyl)phenyl]-1,2-thiazole-5-carboxylate (164-1, 130 mg, 84.60%) as a yellow oil.

Step 2: Synthesis of 4-cyclopropyl-3-(2-(trifluoromethyl)phenyl)-N-(2-(trifluoromethyl)pyridin-4-yl)isothiazole-5-carboxamide (I-164): A solution of ethyl 4-cyclopropyl-3-[2-(trifluoromethyl)phenyl]-1,2-thiazole-5-carboxylate (110 mg, 0.322 mmol, 1 equiv) in THF (2 mL) was treated with 2-(trifluoromethyl)pyridin-4-amine (78.36 mg, 0.483 mmol, 1.5 equiv) followed by the addition of t-BuOK (108.48 mg, 0.966 mmol, 3 equiv) at 0° C. The resulting mixture was stirred for 2 h at room temperature. The reaction was quenched with water at room temperature. The resulting mixture was extracted with EtOAc (3×40 mL). The combined organic layers were washed with brine (40 mL) and dried over anhydrous Na₂SO₄. After filtration, the filtrate was concentrated under reduced pressure. The crude product (70 mg) was purified by prep-HPLC with the following conditions: (column: XBridge Prep OBD C18 column: 30*150 mm, 5 μm; flow rate: 60 mL/min; gradient: 51% B to 76% B in 8 min, 76% B; wavelength: 254 nm; RT1(min): 7.27; injection volume: 0.5 mL; number of runs: 3) to afford 4-cyclopropyl-3-[2-(trifluoromethyl)phenyl]-N-[2-(trifluoromethyl)pyridin-4-yl]-1,2-thiazole-5-carboxamide (1-164, 42.2 mg, 28.54%) as a white solid. LCMS (ES, m/z): [M+H]⁺=458; ¹H NMR (400 MHz, DMSO-d₆) δ 0.30-0.38 (m, 2H), 0.58-0.68 (m, 2H), 1.88 (tt, J=8.6, 5.4 Hz, 1H), 7.58 (d, J=7.6 Hz, 1H), 7.76 (t, J=7.6 Hz, 1H), 7.79-7.87 (m, 1H), 7.90-8.00 (m, 2H), 8.22 (d, J=2.0 Hz, 1H), 8.72 (d, J=5.6 Hz, 1H), 11.36 (s, 1H).

### EXAMPLE 251—SYNTHESIS OF 4-CYCLOPROPYL-3-(PYRIDIN-3-YL)-N-(2-(TRIFLUOROMETHYL)PYRIDIN-4-YL)ISOTHIAZOLE-5-CARBOXAMIDE (I-165)

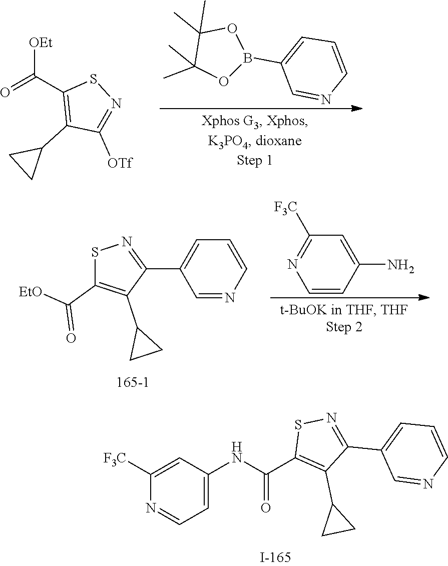

Step 1: Synthesis of 165-1: A mixture of 4-cyclopropyl-5-{[2-(trifluoromethyl)pyridin-4-yl]carbamoyl}-1,2-thiazol-3-yl trifluoromethanesulfonate (100 mg, 0.326 mmol, 1 equiv), 3-(4,4,5,5-tetramethyl-1,3,2-dioxaborolan-2-yl)pyridine (106.68 mg, 0.521 mmol, 1.6 equiv), K₃PO₄ (207 mg, 0.977 mmol, 3 equiv), XPhos Pd G3 (27.53 mg, 0.033 mmol, 0.1 equiv) and XPhos (31 mg. 0.064 mmol. 0.2 equiv) in dioxane (2 mL) was stirred overnight at 60° C. under a nitrogen atmosphere. The desired product could be detected by LCMS. The resulting mixture was filtered, and the filter cake was washed with DCM (3×20 mL). The filtrate was concentrated under reduced pressure. The residue was purified by reversed-phase flash chromatography with the following conditions: column: C18 silica gel; mobile phase: MeCN in water (10 mmol/L NH₄HCO₃), 10% to 50% gradient in 10 min; detector: UV 254 nm. To afford ethyl 4-cyclopropyl-3-(pyridin-3-yl)isothiazole-5-carboxylate (165-1, 90 mg, 75%) as a light brown solid.

Step 2: 4-cyclopropyl-3-(pyridin-3-yl)-N-(2-(trifluoromethyl)pyridin-4-yl)isothiazole-5-carboxamide (I-165): A mixture of ethyl 4-cyclopropyl-3-(pyridin-3-yl)-1,2-thiazole-5-carboxylate (100 mg, 0.365 mmol, 1 equiv), 2-(trifluoromethyl)pyridin-4-amine (88.64 mg, 0.547 mmol, 1.5 equiv) and t-BuOK (122.71 mg, 1.095 mmol, 3 equiv) in THF (2 mL) was stirred for 2 h at room temperature under a nitrogen atmosphere. The reaction was quenched with EtOAc at room temperature. The resulting mixture was filtered, and the filter cake was washed with EtOAc (2×10 mL). The filtrate was concentrated under reduced pressure. The residue was purified by silica gel column chromatography and eluted with PE/EA (1:1) to afford the crude products as a yellow oil. The crude product (50 mg) was purified by prep-HPLC with the following conditions: (column: XBridge Prep OBD C18 column: 30*150 mm, 5 μm; mobile phase A: water (10 mmol/L NH₄HCO₃+0.1% NH₃·H₂O), mobile phase B: ACN; flow rate: 60 mL/min; gradient: 30% B to 59% B in 8 min, 59% B; wavelength: 220 nm; RT1(min): 7.57; injection volume: 0.1 mL; number of runs: 2) to afford 4-cyclopropyl-3-(pyridin-3-yl)-N-[2-(trifluoromethyl)pyridin-4-yl]-1,2-thiazole-5-carboxamide (1-165, 20 mg, 13.97%) as a off-white solid. LCMS (ES, m/z): [M+1]⁺=391.00; ¹H NMR (400 MHz, DMSO-d₆) δ 0.36-0.29 (m, 2H), 0.90-0.81 (m, 2H), 2.27 (tt, J=8.4, 5.5 Hz, 1H), 7.58 (dd, J=7.9, 4.8 Hz, 1H), 7.95 (dd, J=5.5, 2.0 Hz, 1H), 8.22 (d, J=2.0 Hz, 1H), 8.26 (dt, J=8.0, 2.0 Hz, 1H), 8.71 (dd, J=4.8, 1.6 Hz, 1H), 8.73 (d, J=5.5 Hz, 1H), 9.04 (d, J=2.2 Hz, 1H), 11.39 (s, 1H).

### EXAMPLE 252—SYNTHESIS OF 4-CYCLOPROPYL-3-(1H-INDAZOL-5-YL)-N-(2-(TRIFLUOROMETHYL)PYRIDIN-4-YL)ISOTHIAZOLE-5-CARBOXAMIDE (1-166)

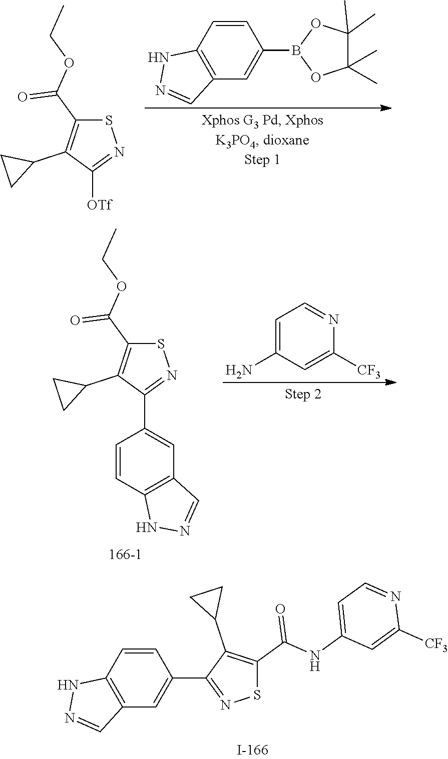

Step 1: Synthesis of 166-1: A solution of ethyl 4-cyclopropyl-3-(1H-indazol-5-yl)-1,2-thiazole-5-carboxylate (200 mg, 0.638 mmol, 1 equiv), 5-(4,4,5,5-tetramethyl-1,3,2-dioxaborolan-2-yl)-1H-indazole (187 mg, 0.766 mmol, 1.2 equiv), XPhos Pd G3 (108 mg, 0.128 mmol, 0.2 equiv), XPhos (76 mg, 0.160 mmol, 0.25 equiv) and K₃PO₄ (406 mg, 1.914 mmol, 3 equiv) in dioxane (2 mL) was stirred for 2 h at 60° C. under a nitrogen atmosphere. The mixture was allowed to cool down to room temperature. The resulting mixture was diluted with water (10 mL). The resulting mixture was extracted with EtOAc (3×10 mL). The combined organic layers were washed with water (1×30 mL) and dried over anhydrous Na₂SO₄. After filtration, the filtrate was concentrated under reduced pressure. The residue was purified by silica gel column chromatography and eluted with PE/EA (5:1) to afford 4-cyclopropyl-3-(1H-indazol-5-yl)-N-[2-(trifluoromethyl) pyridin-4-yl]-1,2-thiazole-5-carboxamide (166-1, 260 mg, 18.97%) as a light yellow oil. LCMS (ES, m/z): [M+H]⁺=314.

Step 2: Synthesis of 4-cyclopropyl-3-(1H-indazol-5-yl)-N-(2-(trifluoromethyl)pyridin-4-yl)isothiazole-5-carboxamide (1-166): To a stirred solution of ethyl 4-cyclopropyl-3-(1H-indazol-5-yl)-1,2-thiazole-5-carboxylate (85 mg, 0.271 mmol, 1 equiv) and 2-(trifluoromethyl)pyridin-4-amine (44 mg, 0.271 mmol, 1 equiv) in THF (2 mL) was added t-BuOK (91 mg, 0.813 mmol, 3 equiv) at 0° C. under a nitrogen atmosphere. The resulting mixture was stirred for 1 h. The resulting mixture was diluted with water (10 mL). The resulting mixture was extracted with EtOAc (3×10 mL). The combined organic layers were washed with water (1×30 mL) and dried over anhydrous Na₂SO₄. After filtration, the filtrate was concentrated under reduced pressure. The crude product was purified by prep-HPLC with the following conditions: column: XBridge Shield RP18 OBD column: 30*150 mm, 5 μm; mobile phase A: water (10 mmol/L NH₄HCO₃+0.1% NH₃·H₂O), mobile phase B: ACN; flow rate: 60 mL/min; gradient: 40% B to 60% B in 8 min, 60% B; wavelength: 220/254 nm; RT1(min): 7.3 to afford 4-cyclopropyl-3-(1H-indazol-5-yl)-N-[2-(trifluoromethyl)pyridin-4-yl]-1,2-thiazole-5-carboxamide (1-166, 6.5 mg, 5.51%) as an off-white solid. LCMS (ES, m/z): [M+H]⁺=430; ¹H NMR (400 MHz, DMSO-d₆) δ 0.20-0.50 (m, 2H), 0.76-0.93 (m, 2H), 2.20-2.40 (m, 1H), 7.66 (d, J=8.8 Hz, 1H), 7.86 (dd, J=8.8, 1.6 Hz, 1H), 7.95 (dd, J=1.6, 6.0 Hz, 1H), 8.17 (s, 1H), 8.25 (d, J=8.8 Hz, 1H), 8.29 (s, 1H), 8.73 (d, J=5.6 Hz, 1H), 11.35 (s, 1H), 13.27 (s, 1H).

### EXAMPLE 253—SYNTHESIS OF 4-CYCLOPROPYL-3-(5-METHYLPYRIDIN-3-YL)-N-(2-(TRIFLUOROMETHYL)PYRIDIN-4-YL)ISOTHIAZOLE-5-CARBOXAMIDE (I-167)

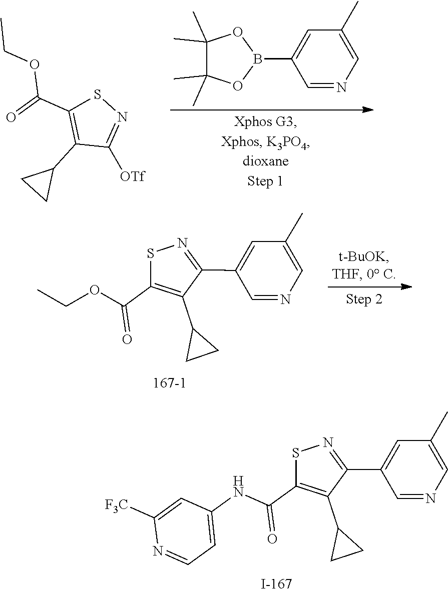 [SMILES: CCOC(=O)c1snc(OS(=O)(=O)C(F)(F)F)c1C1CC1 ; Cc1cncc(B2OC(C)(C)C(C)(C)O2)c1 ; CCOC(=O)c1snc(-c2cncc(C)c2)c1C1CC1 ; Cc1cncc(-c2nsc(C(=O)Nc3ccnc(C(F)(F)F)c3)c2C2CC2)c1]

Step 1: Synthesis of 167-1: To a stirred solution of ethyl 4-cyclopropyl-3-(trifluoromethanesulfonyloxy)-1,2-thiazole-5-carboxylate (200 mg, 0.579 mmol, 1 equiv) and 3-methyl-5-(4,4,5,5-tetramethyl-1,3,2-dioxaborolan-2-yl)pyridine (190 mg, 0.868 mmol, 1.5 equiv) in dioxane (3 mL) was added K₃PO₄ (369 mg, 1.737 mmol, 3 equiv), XPhos (41 mg, 0.087 mmol, 0.15 equiv) and XPhos Pd G3 (49 mg, 0.058 mmol, 0.1 equiv). The resulting mixture was stirred for 2 h at 60° C. under a nitrogen atmosphere. The resulting mixture was diluted with water (20 mL). The resulting mixture was extracted with EtOAc (3×20 mL). The combined organic layers were washed with brine (1×50 mL) and dried over anhydrous Na₂SO₄. After filtration, the filtrate was concentrated under reduced pressure. The residue was purified by prep-TLC (PE/EA 8:1) to afford ethyl 4-cyclopropyl-3-(5-methylpyridin-3-yl)-1,2-thiazole-5-carboxylate (167-1, 150 mg, 53.89%) as a brown yellow solid.

Step 2: Synthesis of 4-cyclopropyl-3-(5-methylpyridin-3-yl)-N-(2-(trifluoromethyl)pyridin-4-yl)isothiazole-5-carboxamide (I-167): A solution of ethyl 4-cyclopropyl-3-(5-methylpyridin-3-yl)-1,2-thiazole-5-carboxylate (100 mg, 0.347 mmol, 1 equiv) in THF (2 mL) was treated with 2-(trifluoromethyl)pyridin-4-amine (73 mg, 0.451 mmol, 1.3 equiv) followed by the addition of t-BuOK (78 mg, 0.694 mmol, 2 equiv) dropwise at 0° C. The resulting mixture was stirred for 1 h at room temperature. The resulting mixture was diluted with water (20 mL). The resulting mixture was extracted with EtOAc (3×20 mL). The combined organic layers were washed with brine (1×50 mL) and dried over anhydrous Na₂SO₄. After filtration, the filtrate was concentrated under reduced pressure. The crude product (40 mg) was purified by prep-HPLC with the following conditions: (column: XBridge Shield RP18 OBD column: 30*150 mm, 5 m; mobile phase A: water (10 mmol/L NH₄HCO₃+0.1% NH₃·H₂O), mobile phase B: ACN; flow rate: 60 mL/min; gradient: 40% B to 57% B in 8 min, 57% B; wavelength: 220/254 nm; RT1(min): 7.27; injection volume: 0.75 mL; number of runs: 3) to afford 4-cyclopropyl-3-(5-methylpyridin-3-yl)-N-[2-(trifluoromethyl)pyridin-4-yl]-1,2-thiazole-5-carboxamide (1-167, 6 mg, 4.25%) as a white solid. LCMS (ES, m/z): [M+1]⁺=405; ¹H NMR (400 MHz, DMSO-d₆) δ 0.29-0.35 (m, 2H), 0.92-0.80 (m, 2H), 2.29-2.23 (m, 1H), 2.43 (s, 31H), 7.94 (dd, J=2.0, 5.6 Hz 1H), 8.14 (s, 1H), 8.22 (d, J=2.0 Hz, 1H), 8.58 (s, 1H), 8.73 (d, J=5.6 Hz, 1H), 8.89 (s, 1H), 11.39 (s, 1H).

### EXAMPLE 254—SYNTHESIS OF 4-CYCLOPROPYL-3-(1-METHYL-1H-INDAZOL-5-YL)-N-(2-(TRIFLUOROMETHYL)PYRIDIN-4-YL)ISOTHIAZOLE-5-CARBOXAMIDE (I-168)

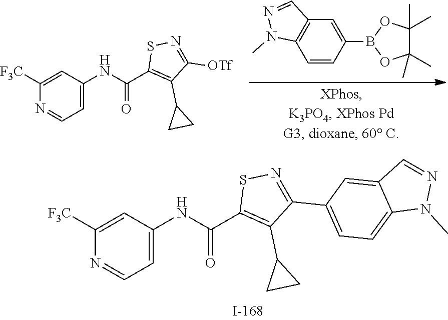

A solution of 4-cyclopropyl-5-{[2-(trifluoromethyl)pyridin-4-yl]carbamoyl}-1,2-thiazol-3-yl trifluoromethanesulfonate (100 mg, 0.217 mmol, 1 equiv) in 1,4-dioxane (2 mL) was treated with 1-methyl-6-(4,4,5,5-tetramethyl-1,3,2-dioxaborolan-2-yl)indazole (67.1 mg, 0.260 mmol, 1.2 equiv) for 5 min at room temperature under a nitrogen atmosphere followed by the addition of K₃PO₄ (138 mg, 0.651 mmol, 3 equiv), XPhos (20.7 mg, 0.043 mmol, 0.2 equiv) and XPhos Pd G3 (73.4 mg, 0.087 mmol, 0.4 equiv) in portions at room temperature. The resulting mixture was stirred for an additional 2 h at 60° C. The resulting mixture was concentrated under reduced pressure. The residue was purified by reversed-phase flash chromatography with the following conditions: column: C18 silica gel; mobile phase: MeCN in water (0.1% FA), 10% to 50% gradient in 20 min; detector: UV 254 nm. This resulted in 4-cyclopropyl-3-(1-methyl-1H-indazol-5-yl)-N-(2-(trifluoromethyl)pyridin-4-yl)isothiazole-5-carboxamide (1-168, 1.7 mg, 1.76%) as a white solid. LCMS (ES, m/z): [M+1]⁺=444; ¹H NMR (400 MHz, DMSO-d₆) δ 0.28-0.36 (m, 2H), 0.81-0.90 (m, 2H), 2.33 (td, J=8.4, 4.2 Hz, 1H), 4.12 (s, 3H), 7.63 (dd, J=8.4, 1.4 Hz, 1H), 7.90 (d, J=8.4 Hz, 1H), 7.96 (dd, J=5.5, 2.0 Hz, 1H), 8.06 (s, 1H), 8.14 (s, 1H), 8.24 (d, J=2.0 Hz, 1H), 8.74 (d, J=5.5 Hz, 1H), 11.42 (s, 1H).

### EXAMPLE 255—SYNTHESIS OF 4-CYCLOPROPYL-3-(1H-INDAZOL-6-YL)-N-(2-(TRIFLUOROMETHYL)PYRIDIN-4-YL)ISOTHIAZOLE-5-CARBOXAMIDE (1-169)

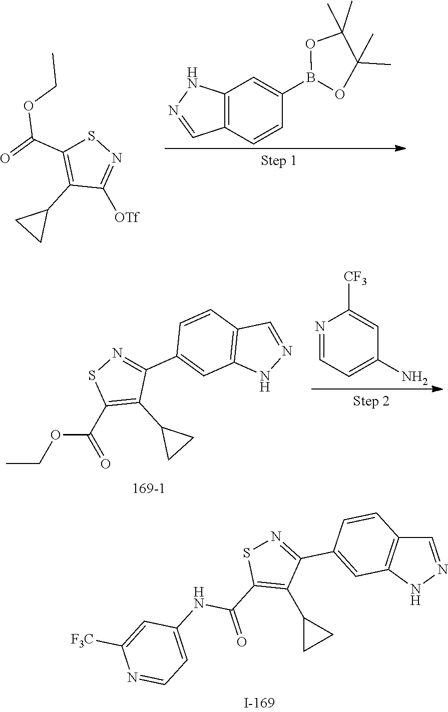

Step 1: Synthesis of 169-1: A mixture of ethyl 4-cyclopropyl-3-(((trifluoromethyl)sulfonyl)oxy)isothiazole-5-carboxylate (350.00 mg, 1.014 mmol, 1 equiv), 6-(4,4,5,5-tetramethyl-1,3,2-dioxaborolan-2-yl)-1H-indazole (296.90 mg, 1.217 mmol, 1.2 equiv), X-Phos (120.80 mg, 0.254 mmol, 0.25 equiv). XPhos Pd G3 (171.59 mg, 0.203 mmol, 0.2 equiv) and K₃PO₄ (645.44 mg, 3.042 mmol, 3 equiv) in dioxane (5 mL) was stirred for 2 h at 60° C. under a nitrogen atmosphere. The resulting mixture was diluted with water (20 mL) and extracted with EtOAc (3×20 mL). The combined organic layers were washed with brine (1×60 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 40% to 50% gradient in 10 min; detector: UV 254 nm, to afford ethyl 4-cyclopropyl-3-(1H-indazol-6-yl)isothiazole-5-carboxylate (169-1, 160.00 mg, 48.91%) as an off-white solid.

Step 2: Synthesis of 4-cyclopropyl-3-(1H-indazol-6-yl)-N-(2-(trifluoromethyl)pyridin-4-yl)isothiazole-5-carboxamide (1-169): To a stirred mixture of ethyl 4-cyclopropyl-3-(1H-indazol-6-yl)isothiazole-5-carboxylate (80.00 mg, 0.275 mmol, 1 equiv) and 2-(trifluoromethyl)pyridin-4-amine (53.41 mg, 0.330 mmol, 1.2 equiv) in THF (3 mL) was added t-BuOK (92.43 mg, 0.825 mmol, 3 equiv) dropwise at room temperature. The resulting mixture was stirred for 30 min at room temperature. The resulting mixture was diluted with water (10 mL) and extracted with EtOAc (3×15 mL). The combined organic layers were washed with brine (1×40 mL) and dried over anhydrous Na₂SO₄. After filtration, the filtrate was concentrated under reduced pressure. The crude product was purified by prep-HPLC with the following conditions: column: XBridge Prep OBD C18 column: 30*150 mm, 5 m; flow rate: 60 mL/min; gradient: 33% B to 58% B in 8 min, 58% B; wavelength: 254 nm; RT1(min): 7.50; injection volume: 0.7 mL, to afford 4-cyclopropyl-3-(1H-indazol-6-yl)-N-(2-(trifluoromethyl)pyridin-4-yl)isothiazole-5-carboxamide (I-169, 36.2 mg, 32.26%) as an off-white solid. LCMS (ES, m/z): [M+H]⁺=430; ¹H NMR (400 MHz, DMSO-d₆) δ 0.29-0.38 (m, 2H), 0.79-0.90 (m, 2H), 2.21-2.30 (m, 1H), 7.60 (dd, J=1.4, 8.4 Hz, 1H), 7.90 (d, J=8.4 Hz, 1H), 7.94 (dd, J=2.0, 4.0 Hz, 1H). 7.99 (d, J=1.4 Hz, 1H), 8.17 (s, 1H), 8.23 (d, J=4.0 Hz, 1H), 8.72 (d, J=4.0 Hz, 1H), 11.35 (s, 1H), 13.27 (s, 1H).

### EXAMPLE 256—SYNTHESIS OF 4-CYCLOPROPYL-3-(4-METHOXYPYRIDIN-3-YL)-N-(2-(TRIFLUOROMETHYL)PYRIDIN-4-YL)ISOTHIAZOLE-5-CARBOXAMIDE (1-170)

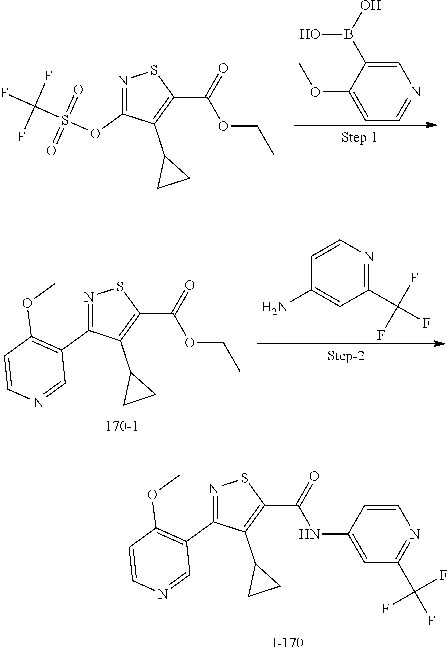 [SMILES: CCOC(=O)c1snc(OS(=O)(=O)C(F)(F)F)c1C1CC1 ; COc1ccncc1B(O)O ; CCOC(=O)c1snc(-c2cnccc2OC)c1C1CC1 ; Nc1ccnc(C(F)(F)F)c1 ; COc1ccncc1-c1nsc(C(=O)Nc2ccnc(C(F)(F)F)c2)c1C1CC1]

Step 1: Synthesis of 170-1: To a stirred mixture of ethyl 4-cyclopropyl-3-(trifluoromethanesulfonyloxy)-1,2-thiazole-5-carboxylate (220 mg, 0.637 mmol, 1 equiv) and 4-methoxypyridin-3-ylboronic acid (97.44 mg, 0.637 mmol, 1 equiv) in dioxane (15 mL) was added XPhos Pd G3 (107.86 mg, 0.127 mmol, 0.2 equiv), XPhos (60.75 mg, 0.127 mmol, 0.2 equiv) and K₃PO₄ (405.71 mg, 1.911 mmol, 3 equiv) in portions at room temperature under a nitrogen atmosphere. The mixture was stirred for 12 h at 60° C. under a nitrogen atmosphere. The resulting mixture was extracted with EtOAc (3×100 mL). The combined organic layers were washed with brine (100 mL) and dried over anhydrous Na₂SO₄. After filtration, the filtrate was concentrated under reduced pressure. The residue was purified by prep-TLC (PE/EA 5:1) to afford 170-1 (70 mg, 36.10%) as a brown yellow crude oil.

Step 2: Synthesis of 4-cyclopropyl-3-(4-methoxypyridin-3-yl)-N-(2-(trifluoromethyl)pyridin-4-yl)isothiazole-5-carboxamide (I-170): A solution of 170-1 (70 mg, 0.230 mmol, 1 equiv) in THF (10 mL) was treated with 2-(trifluoromethyl)pyridin-4-amine (44.74 mg, 0.276 mmol, 1.2 equiv) for 20 min at 0° C. under a nitrogen atmosphere followed by the addition of t-BuOK (77.42 mg, 0.690 mmol, 3 equiv) at 0° C. The resulting mixture was stirred for 20 min at 0° C. under a nitrogen atmosphere. The mixture was acidified to pH 5 with HCl (aq). The aqueous layer was extracted with EtOAc (6×100 mL). The crude product (112 mg) was purified by prep-HPLC with the following conditions: (column: XBridge Prep OBD C18 column: 30*150 mm, 5 μm; mobile phase A: water (10 mmol/L NH₄HCO₃), mobile phase B: ACN; flow rate: 60 mL/min; gradient: 34% B to 54% B in 8 min, 54% B; wavelength: 254 nm; RT1(min): 7.48; injection volume: 1 mL) to afford 4-cyclopropyl-3-(4-methoxypyridin-3-yl)-N-(2-(trifluoromethyl)pyridin-4-yl)isothiazole-5-carboxamide (I-170, 11.1 mg, 11.30%) as a white solid. LCMS (ES, m/z): [M+H]⁺=42; ¹H NMR (400 MHz, DMSO-d₆): δ 0.25 (d, J=4.0 Hz, 2H), 0.63 (dd, J=1.6, 8.4 Hz, 2H), 1.98 (s, 1H), 3.91 (s, 3H), 7.26 (d, J=6.0 Hz, 1H), 7.96 (d, J=5.6 Hz, 1H), 8.22 (d, J=2.0 Hz, 1H), 8.37 (s, 1H), 8.59 (d, J=6.0 Hz, 1H), 8.72 (d, J=5.6 Hz, 1H), 11.37 (s, 1H).

### EXAMPLE 256—SYNTHESIS OF 4-CYCLOPROPYL-3-(2-METHYLPYRIDIN-3-YL)-N-(2-(TRIFLUOROMETHYL)PYRIDIN-4-YL)ISOTHIAZOLE-5-CARBOXAMIDE (I-171)

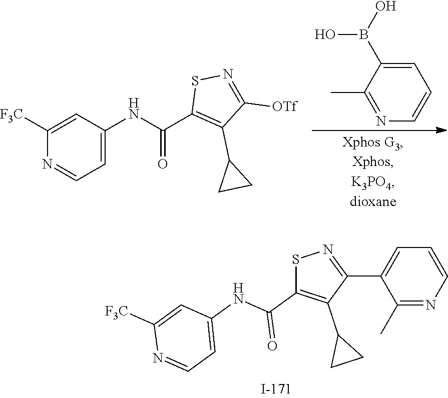

A solution of 4-cyclopropyl-5-{[2-(trifluoromethyl)pyridin-4-yl]carbamoyl}-1,2-thiazol-3-yl trifluoromethanesulfonate (100 mg, 0.217 mmol, 1 equiv), 2-methylpyridin-3-ylboronic acid (59.37 mg, 0.434 mmol, 2 equiv), K₃PO₄ (138.03 mg, 0.651 mmol, 3 equiv), X-Phos (10.33 mg, 0.022 mmol, 0.1 equiv) and XPhos Pd G3 (18.35 mg, 0.022 mmol, 0.1 equiv) in dioxane (5 mL) was stirred overnight at 60° C. under a nitrogen atmosphere. The reaction was quenched with water at room temperature. The resulting mixture was extracted with EtOAc (2×20 mL). The combined organic layers were washed with brine (1×10 mL), dried over anhydrous MgSO₄. After filtration, the filtrate was concentrated under reduced pressure. The residue was purified by reversed-phase flash chromatography with the following conditions: column: C18 silica gel; mobile phase: MeCN in water (0.1% FA), 40% to 50% gradient in 10 min; detector: UV 254 nm. This resulted in 4-cyclopropyl-3-(2-methylpyridin-3-yl)-N-[2-(trifluoromethyl) pyridin-4-yl]-1,2-thiazole-5-carboxamide (I-171, 1.5 mg, 1.71%) as an off-white solid. LCMS (ES, m/z): [M+H]⁺ =405; ¹H NMR (400 MHz, DMSO-d₆): δ 0.28-0.36 (m, 2H), 0.62-0.72 (m, 2H), 1.96 (m, J=14.0, 8.6, 5.4 Hz, 1H), 2.40 (s, 3H), 7.38 (dd, J=7.8, 4.8 Hz, 1H), 7.78 (dd, J=7.8, 1.8 Hz, 1H), 7.94 (dd, J=5.4, 2.0 Hz, 1H), 8.22 (d, J=2.0 Hz, 1H), 8.58 (dd, J=4.9, 1.8 Hz, 1H), 8.72 (d, J=5.6 Hz, 1H), 11.22 (s, 1H).

### EXAMPLE 257—SYNTHESIS OF 4-CYCLOPROPYL-3-(4-FLUOROPHENYL)-N-(2-(TRIFLUOROMETHYL)PYRIDIN-4-YL)ISOTHIAZOLE-5-CARBOXAMIDE (I-172)

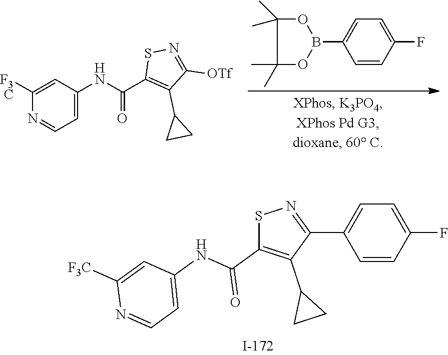 [SMILES: O=C(Nc1ccnc(C(F)(F)F)c1)c1snc(OS(=O)(=O)C(F)(F)F)c1C1CC1 ; CC1(C)OB(c2ccc(F)cc2)OC1(C)C ; O=C(Nc1ccnc(C(F)(F)F)c1)c1snc(-c2ccc(F)cc2)c1C1CC1]

To a stirred solution of 4-cyclopropyl-5-{[2-(trifluoromethyl) pyridin-4-yl]carbamoyl}-1,2-thiazol-3-yl trifluoromethanesulfonate (90 mg, 0.195 mmol, 1 equiv) and 2-(4-fluorophenyl)-4,4,5,5-tetramethyl-1,3,2-dioxaborolane (51.98 mg, 0.234 mmol, 1.2 equiv) in 1,4-dioxane (2 mL) was added XPhos (9.30 mg, 0.020 mmol, 0.1 equiv), XPhos Pd G3 (16.51 mg, 0.020 mmol, 0.1 equiv) and K₃PO₄ (103.52 mg, 0.488 mmol, 2.5 equiv) in portions at room temperature under a nitrogen atmosphere. The resulting mixture was stirred for an additional 2 h at 60° C. The resulting mixture was diluted with water (20 mL). The resulting mixture was extracted with EtOAc (3×30 mL). The combined organic layers were washed with brine (1×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 45% to 55% gradient in 10 min; detector: UV 254 nm. The resulting mixture was concentrated under reduced pressure. This resulted in 4-cyclopropyl-3-(4-fluorophenyl)-N-[2-(trifluoromethyl) pyridin-4-yl]-1,2-thiazole-5-carboxamide (I-172, 8.1 mg, 10.18%) as a white solid. LCMS (ES, m/z): [M+H]⁺=408; ¹H NMR (400 MHz, DMSO-d₆) δ 0.31 (q, J=5.4 Hz, 2H), 0.80-0.91 (m, 2H), 2.17-2.27 (m, 1H), 7.37 (t, J=8.8 Hz, 2H), 7.86-8.02 (m, 3H), 8.23 (d, J=2.0 Hz, 1H), 8.73 (d, J=5.6 Hz, 1H), 11.38 (s, 1H).

### EXAMPLE 258-4-CYCLOPROPYL-3-(4-METHYLPYRIDIN-3-YL)-N-(2-(TRIFLUOROMETHYL)PYRIDIN-4-YL)ISOTHIAZOLE-5-CARBOXAMIDE (I-173)

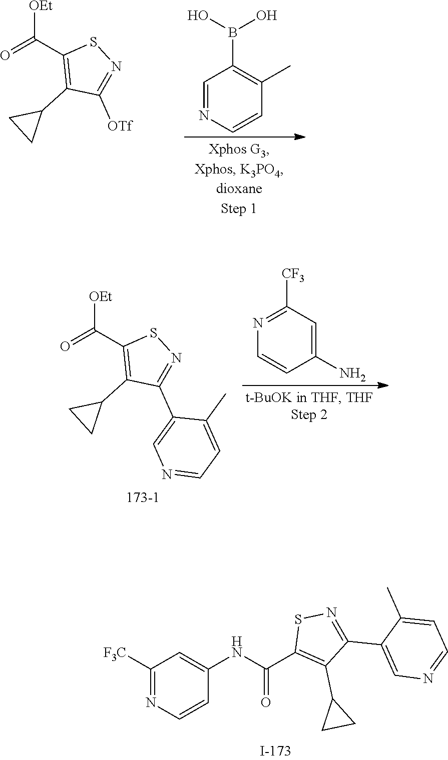

Step 1: Synthesis of 173-1: A solution of ethyl 4-cyclopropyl-3-(trifluoromethanesulfonyloxy)-1,2-thiazole-5-carboxylate (150 mg, 0.434 mmol, 1 equiv) and 4-methylpyridin-3-ylboronic acid (72 mg, 0.526 mmol, 1.21 equiv), XPhos (42 mg, 0.088 mmol, 0.20 equiv), XPhos Pd G3 (74 mg, 0.087 mmol, 0.20 equiv) and K₃PO₄ (276 mg, 1.300 mmol, 2.99 equiv) in 1,4-dioxane (10 mL) was stirred overnight at 60° C. under a nitrogen atmosphere. The resulting mixture was diluted with water (10 mL). The resulting mixture was extracted with EtOAc (3×20 mL). The combined organic layers were washed with brine (1×40 mL) and dried over anhydrous Na₂SO₄. After filtration, the filtrate was concentrated under reduced pressure. The crude product (80 mg) was purified by prep-HPLC with the following conditions: (column: XBridge Prep OBD C18 column: 30*150 mm, 5 μm; mobile phase A: water (10 mmol/L NH₄HCO₃+0.1% NH₃·H₂O), mobile phase B: ACN; flow rate: 60 mL/min; gradient: 36% B to 65% B in 8 min, 65% B; wavelength: 220 nm; RT1(min): 7.47; number of runs: 1) to afford ethyl 4-cyclopropyl-3-(4-methylpyridin-3-yl)-1,2-thiazole-5-carboxylate (173-1, 90 mg, 71.85%) as a light yellow oil.

Step 2: Synthesis of 4-cyclopropyl-3-(4-methylpyridin-3-yl)-N-(2-(trifluoromethyl)pyridin-4-yl)isothiazole-5-carboxamide (I-173): A solution of ethyl 4-cyclopropyl-3-(4-methylpyridin-3-yl)-1,2-thiazole-5-carboxylate (90 mg, 0.312 mmol, 1 equiv), 2-(trifluoromethyl)pyridin-4-amine (61 mg, 0.376 mmol, 1.21 equiv) and t-BuOK (106 mg, 0.945 mmol, 3.03 equiv) in THF (5 mL) was stirred for 0.5 h at room temperature under a nitrogen atmosphere. The resulting mixture was diluted with water (5 mL). The resulting mixture was extracted with EtOAc (3×10 mL). The combined organic layers were washed with brine (1×4 mL) and dried over anhydrous Na₂SO₄. After filtration, the filtrate was concentrated under reduced pressure. The crude product (50 mg) was purified by prep-HPLC with the following conditions: (column: Xselect CSH C18 OBD column 30*150 mm 5 m; mobile phase A: water (0.1% FA), mobile phase B: ACN; flow rate: 60 mL/min; gradient: 31% B to 41% B in 7 min, 41% B; wavelength: 254; 220 nm; RT1(min): 5.65, 8.32) to afford 4-cyclopropyl-3-(4-methylpyridin-3-yl)-N-(2-(trifluoromethyl)pyridin-4-yl)isothiazole-5-carboxamide (1-173, 3.3 mg, 2.61%) as an off-white solid. LCMS (ES, m/z): [M+H]⁺ =405; ¹H NMR (400 MHz, DMSO-d₆) δ 0.11-0.47 (m, 2H), 0.51-0.80 (m, 2H), 1.91-2.08 (m, 1H), 2.26 (s. 3H), 7.43 (d, J=5.2 Hz, 1H), 7.95 (dd, J=5.2, 1.2 Hz, 1H), 8.22 (d, J=1.6 Hz, 1H), 8.51 (s, 1H), 8.55 (d, J=5.2 Hz, 1H), 8.73 (d, J=4.8 Hz, 1H), 11.36 (s, 1H).

### EXAMPLE 259—SYNTHESIS OF 4-CYCLOPROPYL-3-(3-(DIFLUOROMETHYL) PHENYL)-N-(2-(TRIFLUOROMETHYL)PYRIDIN-4-YL)ISOTHIAZOLE-5-CARBOXAMIDE (1-174)

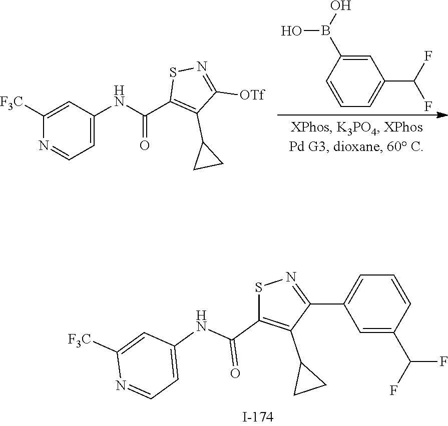 [SMILES: O=C(Nc1ccnc(C(F)(F)F)c1)c1snc(OS(=O)(=O)C(F)(F)F)c1C1CC1 ; OB(O)c1cccc(C(F)F)c1 ; O=C(Nc1ccnc(C(F)(F)F)c1)c1snc(-c2cccc(C(F)F)c2)c1C1CC1]

To a stirred solution of 4-cyclopropyl-5-{[2-(trifluoromethyl) pyridin-4-yl]carbamoyl}-1,2-thiazol-3-yl trifluoromethanesulfonate (90 mg, 0.195 mmol, 1 equiv) and [3-(difluoromethyl)phenyl]boronic acid (40.25 mg, 0.234 mmol, 1.2 equiv) in 1,4-dioxane (2 mL) was added XPhos (9.30 mg, 0.020 mmol, 0.1 equiv), XPhos Pd G3 (16.51 mg, 0.020 mmol, 0.1 equiv) and K₃PO₄ (103.52 mg, 0.488 mmol, 2.5 equiv) in portions at room temperature under a nitrogen atmosphere. The resulting mixture was stirred for an additional 2 h at 60° C. The resulting mixture was diluted with water (10 mL). The resulting mixture was extracted with EtOAc (3×20 mL). The combined organic layers were washed with brine (1×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 50% to 60% gradient in 10 min; detector: UV 254 nm. The resulting mixture was concentrated under reduced pressure. This resulted in 4-cyclopropyl-3-[3-(difluoromethyl)phenyl]-N-[2-(trifluoromethyl) pyridin-4-yl]-1,2-thiazole-5-carboxamide (I-174, 6.7 mg, 7.81%) as a white solid. LCMS (ES, m/z): [M+H]⁺=440; ¹H NMR (400 MHz, DMSO-d₆) δ 0.32 (d, J=5.6 Hz, 2H), 0.85 (d, J=8.2 Hz, 2H), 2.20-2.31 (m, 1H), 7.17 (t, J=55.8 Hz, 1H), 7.71 (d, J=8.0 Hz, 2H), 7.93 (d, J=5.6 Hz, 1H), 8.06 (d, J=6.0 Hz, 2H), 8.22 (s, 1H), 8.71 (d, J=5.6 Hz, 1H), 10.33 (s, 1H).

### EXAMPLE 260—SYNTHESIS OF 4-CYCLOPROPYL-3-(1-METHYL-1H-PYRROLO [2,3-B]PYRIDIN-4-YL)-N-(2-(TRIFLUOROMETHYL)PYRIDIN-4-YL) ISOTHIAZOLE-5-CARBOXAMIDE (I-175)

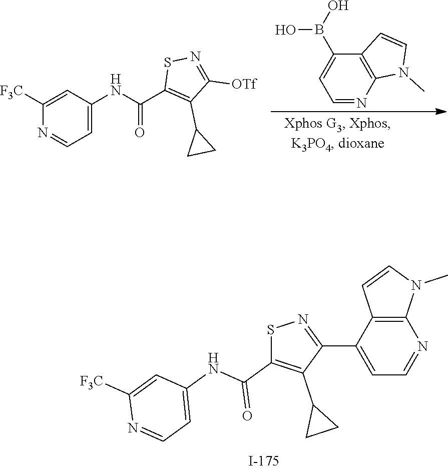 [SMILES: O=C(Nc1ccnc(C(F)(F)F)c1)c1snc(OS(=O)(=O)C(F)(F)F)c1C1CC1 ; Cn1ccc2c(B(O)O)ccnc21 ; Cn1ccc2c(-c3nsc(C(=O)Nc4ccnc(C(F)(F)F)c4)c3C3CC3)ccnc21]

A solution of 4-cyclopropyl-5-{[2-(trifluoromethyl)pyridin-4-yl]carbamoyl}-1,2-thiazol-3-yl trifluoromethanesulfonate (100 mg, 0.217 mmol, 1 equiv), 1-methylpyrrolo[2,3-b]pyridin-4-ylboronic acid (57.22 mg, 0.326 mmol, 1.5 equiv), K₃PO₄ (138.03 mg, 0.651 mmol, 3 equiv), XPhos (10.33 mg, 0.022 mmol, 0.1 equiv) and XPhos Pd G3 (18.35 mg, 0.022 mmol, 0.1 equiv) in dioxane (5 mL) was stirred overnight at 60° C. under a nitrogen atmosphere. The reaction was quenched with water at room temperature. The resulting mixture was extracted with EtOAc (2×20 mL). The combined organic layers were washed with brine (1×10 mL) and dried over anhydrous MgSO₄. The residue was purified by reversed-phase flash chromatography with the following conditions: column: C18 silica gel; mobile phase: MeCN in water (0.1% TFA); 40% to 50% gradient in 10 min; detector: UV 254 nm. This resulted in 4-cyclopropyl-3-{1-methylpyrrolo[2,3-b]pyridin-4-yl}-N-[2-(trifluoromethyl) pyridin-4-yl]-1,2-thiazole-5-carboxamide (I-175, 13.7 mg, 14.25%) as an off-white solid. LCMS (ES, m/z): [M+H]⁺ =444; ¹H NMR (400 MHz, DMSO-d₆): δ 0.26-0.36 (m, 2H), 0.68-0.78 (m, 2H), 2.22-2.51 (m, 1H), 3.90 (s, 3H), 6.66 (d, J=3.6 Hz, 1H), 7.48 (d, J=5.0 Hz, 1H), 7.64 (d, J=3.6 Hz, 1H), 7.94 (dd, J=5.6, 2.0 Hz, 1H), 8.24 (d, J=2.0 Hz, 1H), 8.42 (d, J=5.0 Hz, 1H), 8.72 (d, J=5.6 Hz, 1H), 10.36 (s, 1H).

### EXAMPLE 261—SYNTHESIS OF 4-CYCLOPROPYL-3-(4-(METHYLCARBAMOYL) PHENYL)-N-(2-(TRIFLUOROMETHYL)PYRIDIN-4-YL)ISOTHIAZOLE-5-CARBOXAMIDE (I-176)

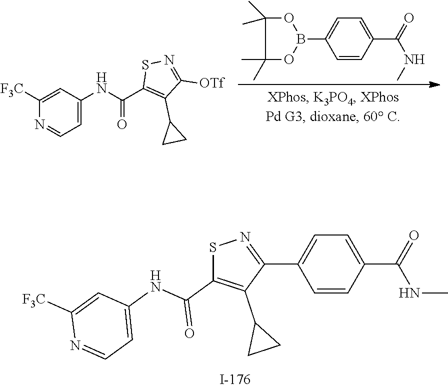 [SMILES: O=C(Nc1ccnc(C(F)(F)F)c1)c1snc(OS(=O)(=O)C(F)(F)F)c1C1CC1 ; CNC(=O)c1ccc(B2OC(C)(C)C(C)(C)O2)cc1 ; CNC(=O)c1ccc(-c2nsc(C(=O)Nc3ccnc(C(F)(F)F)c3)c2C2CC2)cc1]

To a stirred solution of 4-cyclopropyl-5-{[2-(trifluoromethyl) pyridin-4-yl]carbamoyl}-1,2-thiazol-3-yl trifluoromethanesulfonate (90 mg, 0.195 mmol, 1 equiv) and N-methyl-4-(4,4,5,5-tetramethyl-1,3,2-dioxaborolan-2-yl)benzamide (61.13 mg, 0.234 mmol, 1.2 equiv) in 1,4-dioxane (2 mL) was added XPhos (9.30 mg, 0.020 mmol, 0.1 equiv), XPhos Pd G3 (16.51 mg, 0.020 mmol, 0.1 equiv) and K₃PO₄ (103.52 mg, 0.488 mmol, 2.5 equiv) in portions at room temperature under a nitrogen atmosphere. The resulting mixture was stirred for an additional 2 h at 60° C. The resulting mixture was diluted with water (10 mL). The resulting mixture was extracted with EtOAc (3×20 mL). The combined organic layers were washed with brine (1×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 55% to 65% gradient in 10 min; detector: UV 254 nm. The resulting mixture was concentrated under reduced pressure. This resulted in 4-cyclopropyl-3-[4-(methylcarbamoyl)phenyl]-N-[2-(trifluoromethyl) pyridin-4-yl]-1,2-thiazole-5-carboxamide (I-176, 13.8 mg, 15.78%) as a white solid. LCMS (ES, m/z): [M+H]⁺=447; ¹H NMR (400 MHz, DMSO-d₆) δ 0.30 (dd, J=6.0, 4.6 Hz, 2H), 0.81-0.87 (m, 2H), 2.23-2.28 (m, 1H), 2.82 (d, J=4.6 Hz, 3H), 7.92-7.99 (m, J=7.8 Hz, 5H), 8.22 (d, J=2.0 Hz, 1H), 8.57 (d, J=4.6 Hz, 1H), 8.73 (d, J=5.6 Hz, 1H), 11.38 (s, 1H).

### EXAMPLE 262—SYNTHESIS OF 4-CYCLOPROPYL-3-(ISOQUINOLIN-4-YL)-N-(2-(TRIFLUOROMETHYL)PYRIDIN-4-YL)ISOTHIAZOLE-5-CARBOXAMIDE (1-177)

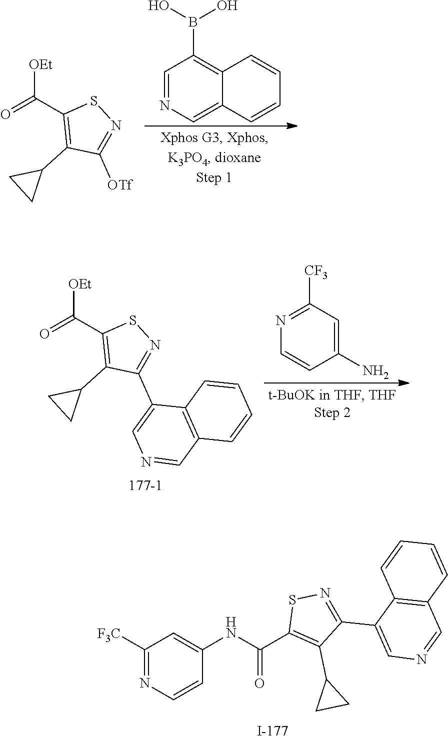 [SMILES: CCOC(=O)c1snc(OS(=O)(=O)C(F)(F)F)c1C1CC1 ; OB(O)c1cncc2ccccc12 ; CCOC(=O)c1snc(-c2cncc3ccccc23)c1C1CC1 ; Nc1ccnc(C(F)(F)F)c1 ; O=C(Nc1ccnc(C(F)(F)F)c1)c1snc(-c2cncc3ccccc23)c1C1CC1]

Step 1: Synthesis of 177-1: A solution of ethyl 4-cyclopropyl-3-(trifluoromethanesulfonyloxy)-1,2-thiazole-5-carboxylate (150 mg, 0.434 mmol, 1 equiv), isoquinolin-4-yl boronic acid (90 mg, 0.520 mmol, 1.20 equiv), XPhos (42 mg, 0.088 mmol, 0.20 equiv), XPhos Pd G3 (74 mg, 0.087 mmol, 0.20 equiv) and K₃PO₄ (276 mg, 1.300 mmol, 2.99 equiv) in 1,4-dioxane (10 mL) was stirred overnight at 60° C. under a nitrogen atmosphere. The resulting mixture was diluted with water (10 mL). The resulting mixture was extracted with EtOAc (3×20 mL). The combined organic layers were washed with brine (1×40 mL) and dried over anhydrous Na₂SO₄. After filtration, the filtrate was concentrated under reduced pressure. The crude product (80 mg) was purified by prep-HPLC with the following conditions: (column: XBridge Prep OBD C18 column: 30*150 mm, 5 μm; mobile phase A: water (10 mmol/L NH₄HCO-+0.1% NH₃·H₂O), mobile phase B: ACN; flow rate: 60 mL/min; gradient: 36% B to 65% B in 8 min, 65% B; wavelength: 220 nm; RT1(min): 7.47: number of runs: 1) to afford ethyl 4-cyclopropyl-3-(isoquinolin-4-yl)-1,2-thiazole-5-carboxylate (177-1, 80 mg, 56.77%) as a light yellow oil.

Step 2: Synthesis of 4-cyclopropyl-3-(isoquinolin-4-yl)-N-(2-(trifluoromethyl)pyridin-4-yl)isothiazole-5-carboxamide (I-177): A solution of ethyl 4-cyclopropyl-3-(isoquinolin-4-yl)-1,2-thiazole-5-carboxylate (80 mg, 0.247 mmol, 1 equiv) and 2-(trifluoromethyl)pyridin-4-amine (48 mg, 0.296 mmol, 1.20 equiv) and t-BuOK (83 mg, 0.740 mmol, 3.00 equiv) in THF (5 mL) was stirred for 0.5 h at room temperature under a nitrogen atmosphere. The resulting mixture was diluted with water (5 mL). The resulting mixture was extracted with EtOAc (3×10 mL). The combined organic layers were washed with brine (1×4 mL) and dried over anhydrous Na₂SO₄. After filtration, the filtrate was concentrated under reduced pressure. The crude product (50 mg) was purified by prep-HPLC with the following conditions: (column: Xselect CSH C18 OBD column 30*150 mm 5 μm; mobile phase A: water (0.1% FA), mobile phase B: ACN; flow rate: 60 mL/min; gradient: 31% B to 41% B in 7 min, 41% B; wavelength: 254; 220 nm; RT1(min): 5.65, 8.32) to afford 4-cyclopropyl-3-(isoquinolin-4-yl)-N-[2-(trifluoromethyl)pyridin-4-yl]-1,2-thiazole-5-carboxamide (1-177, 25.2 mg, 23.18%) as an off-white solid. LCMS (ES, m/z): [M+H]⁺=441; ¹H NMR (400 MHz, DMSO-d₆) δ 0.10-0.38 (m, 2H), 0.39-0.70 (m, 2H), 1.82-2.10 (m, 1H), 7.75-7.82 (m, 2H), 7.83-7.90 (m, 1H), 7.97 (dd, J=5.6, 2.0 Hz, 1H), 8.24 (d, J=2.0 Hz, 1H), 8.26-8.32 (m, 1H), 8.63 (s, 1H), 8.74 (d, J=5.2 Hz, 1H), 9.48 (s, 1H), 11.41 (br s, 1H).

### EXAMPLE 263—SYNTHESIS OF 4-CYCLOPROPYL-3-(M-TOLYL)-N-(2-(TRIFLUOROMETHYL)PYRIDIN-4-YL)ISOTHIAZOLE-5-CARBOXAMIDE (I-178)

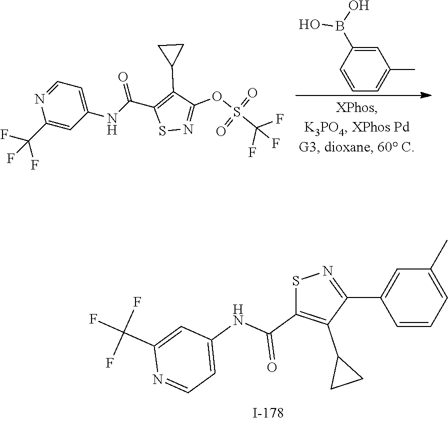

A solution of 4-cyclopropyl-5-{[2-(trifluoromethyl)pyridin-4-yl]carbamoyl}-1,2-thiazol-3-yl trifluoromethanesulfonate (100.00 mg, 0.22 mmol, 1.00 equiv), 3-methylphenylboronic acid (58.9 mg, 0.44 mmol, 2 equiv), XPhos (10.3 mg, 0.02 mmol, 0.1 equiv), XPhos Pd G3 (18.3 mg, 0.02 mmol, 0.1 equiv) and K₃PO₄ (92.0 mg, 0.44 mmol, 2 equiv) in dioxane (5 mL) was stirred for 12 h at 60° C. under a nitrogen atmosphere. The resulting mixture was diluted with water (30 mL). The resulting mixture was extracted with EtOAc (3×50 mL). The combined organic layers were washed with brine (1×10 mL) and dried over anhydrous Na₂SO₄. After filtration, the filtrate was concentrated under reduced pressure. The crude product (30 mg) was purified by prep-HPLC with the following conditions: (column: XSelect CSH Fluoro Phenyl, 30*150 mm, 5 m; mobile phase A: water (0.1% FA), mobile phase B: ACN; flow rate: 60 mL/min; gradient: 45% B to 55% B in 8 min, 55% B; wavelength: 254; 220 nm; RT1(min): 7.98; number of runs: 2) to afford 4-cyclopropyl-3-(3-methylphenyl)-N-[2-(trifluoromethyl)pyridin-4-yl]-1,2-thiazole-5-carboxamide (1-178, 1.9 mg, 21.16%) as a white solid. LCMS (ES, m/z): [M+H]⁺=404; ¹H NMR (400 MHz, DMSO-d₆) δ 0.31 (m, J=6.0, 4.4 Hz, 2H), 0.79-0.88 (m, 2H), 2.16-2.29 (m, 1H), 2.41 (s, 3H), 7.32 (d, J=7.6 Hz, 1H), 7.42 (t, J=8.0 Hz, 1H), 7.64 (d, J=6.8 Hz, 2H), 7.94 (dd, J=5.6, 2.0 Hz, 1H), 8.22 (d, J=2.0 Hz, 1H), 8.73 (d, J=5.6 Hz, 1H), 11.37 (s, 1H).

### EXAMPLE 264—SYNTHESIS OF 4-CYCLOPROPYL-3-(1-METHYL-1H-INDAZOL-5-YL)-N-(2-(TRIFLUOROMETHYL)PYRIDIN-4-YL)ISOTHIAZOLE-5-CARBOXAMIDE (I-179)

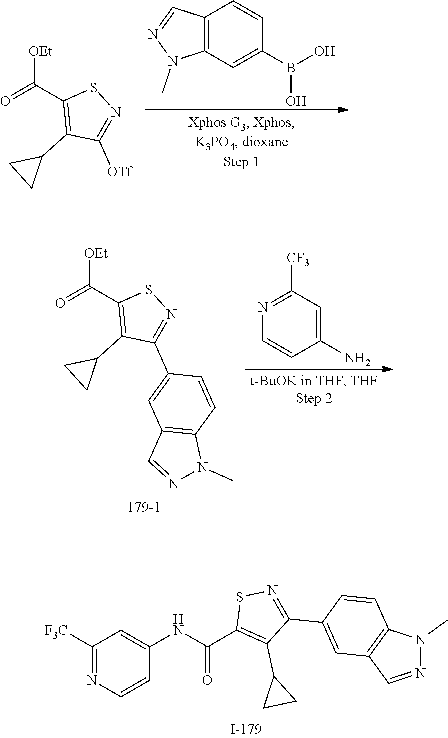 [SMILES: CCOC(=O)c1snc(OS(=O)(=O)C(F)(F)F)c1C1CC1 ; Cn1ncc2ccc(B(O)O)cc21 ; CCOC(=O)c1snc(-c2ccc3c(cnn3C)c2)c1C1CC1 ; Nc1ccnc(C(F)(F)F)c1 ; Cn1ncc2cc(-c3nsc(C(=O)Nc4ccnc(C(F)(F)F)c4)c3C3CC3)ccc21]

Step 1: Synthesis of 179-1: A solution of ethyl 4-cyclopropyl-3-(trifluoromethanesulfonyloxy)-1,2-thiazole-5-carboxylate (150 mg, 0.434 mmol, 1 equiv), 1-methylindazol-6-ylboronic acid (92 mg, 0.523 mmol, 1.20 equiv), XPhos (42 mg, 0.088 mmol, 0.20 equiv), XPhos Pd G3 (74 mg, 0.087 mmol, 0.20 equiv) and K₃PO₄ (276 mg, 1.300 mmol, 2.99 equiv) in 1,4-dioxane (10 mL) was stirred overnight at 60° C. under a nitrogen atmosphere. The resulting mixture was diluted with water (10 mL). The resulting mixture was extracted with EtOAc (3×20 mL). The combined organic layers were washed with brine (1×40 mL) and dried over anhydrous Na₂SO₄. After filtration, the filtrate was concentrated under reduced pressure. The crude product (80 mg) was purified by prep-HPLC with the following conditions: (column: XBridge Prep OBD C18 column: 30*150 mm, 5 μm; mobile phase A: water (10 mmol/L NH₄HCO₃+0.1% NH₃·H₂O), mobile phase B: ACN; flow rate: 60 mL/min; gradient: 36% B to 65% B in 8 min, 65% B; wavelength: 220 nm; RT1(min): 7.47; number of runs: 1) to afford ethyl 4-cyclopropyl-3-(1-methylindazol-6-yl)-1,2-thiazole-5-carboxylate (179-1, 80 mg, 56.25%) as a light yellow oil.

Step 2: Synthesis of 4-cyclopropyl-3-(1-methyl-1H-indazol-5-yl)-N-(2-(trifluoromethyl)pyridin-4-yl)isothiazole-5-carboxamide (I-179): A solution of ethyl 4-cyclopropyl-3-(1-methylindazol-6-yl)-1,2-thiazole-5-carboxylate (80 mg, 0.244 mmol, 1 equiv), 2-(trifluoromethyl) pyridin-4-amine (48 mg, 0.296 mmol, 1.21 equiv) and t-BuOK (82 mg, 0.731 mmol, 2.99 equiv) in THF (5 mL) was stirred for 0.5 h at room temperature under a nitrogen atmosphere. The resulting mixture was diluted with water (5 mL). The resulting mixture was extracted with EtOAc (3×10 mL). The combined organic layers were washed with brine (1×20 mL) and dried over anhydrous Na₂SO₄. After filtration, the filtrate was concentrated under reduced pressure. The crude product (50 mg) was purified by prep-HPLC with the following conditions: (column: Xselect CSH C18 OBD column 30*150 mm 5 m; mobile phase A: water (0.1% FA), mobile phase B: ACN; flow rate: 60 mL/min; gradient: 31% B to 41% B in 7 min, 41% B; wavelength: 254; 220 nm; RT1(min): 5.65, 8.32) to afford 4-cyclopropyl-3-(1-methylindazol-5-yl)-N-[2-(trifluoromethyl)pyridin-4-yl]-1,2-thiazole-5-carboxamide (I-179, 29.4 mg, 26.92%) as an off-white solid. LCMS (ES, m/z): [M+1]⁺=444; ¹H NMR (400 MHz, DMSO-d₆) δ 0.15-0.45 (m, 2H), 0.69-0.98 (m, 2H), 2.22-2.31 (m, 1H), 4.11 (s, 3H), 7.77 (d, J=8.8 Hz, 1H), 7.83-7.91 (m, 1H), 7.92-8.01 (m, 1H), 8.19 (s, 1H), 8.21-8.29 (m, 2H), 8.73 (d, J=5.6 Hz, 1H), 11.37 (br s, 1H).

### EXAMPLE 265—SYNTHESIS OF N-(3-CYANO-4-(3-HYDROXYAZETIDINE-1-CARBONYL)PHENYL)-4-CYCLOPROPYL-3-(QUINOLIN-5-YL)ISOTHIAZOLE-5-CARBOXAMIDE (I-180)

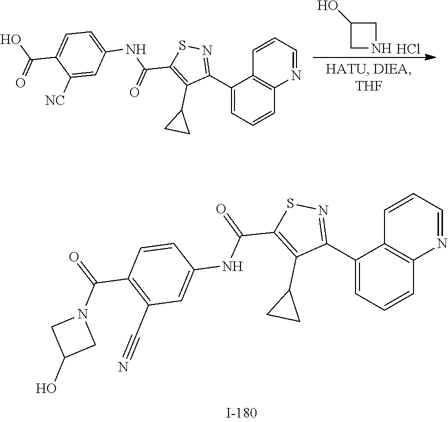 [SMILES: N#Cc1cc(NC(=O)c2snc(-c3cccc4ncccc34)c2C2CC2)ccc1C(=O)O ; OC1CNC1 ; Cl ; N#Cc1cc(NC(=O)c2snc(-c3cccc4ncccc34)c2C2CC2)ccc1C(=O)N1CC(O)C1]

A solution of 2-cyano-4-[4-cyclopropyl-3-(quinolin-5-yl)-1,2-thiazole-5-amido]benzoic acid (25.1 mg, 0.057 mmol, 1 equiv) in THF (1 mL) was treated with HATU (32.50 mg, 0.086 mmol, 1.5 equiv) and DIEA (36.82 mg, 0.285 mmol, 5 equiv) for 30 min at room temperature under a nitrogen atmosphere, followed by the addition of azetidin-3-ol hydrochloride (6.87 mg, 0.063 mmol, 1.1 equiv) in portions at room temperature. The mixture was stirred for 2 h at room temperature under a nitrogen atmosphere. The aqueous layer was extracted with EtOAc (3×50 mL). After filtration, the filtrate was concentrated under reduced pressure. The crude product was purified by prep-HPLC with the following conditions: (column: XBridge Prep OBD C18 column: 30*150 mm, 5 m; flow rate: 60 mL/min; gradient: 32% B to 62% B in 7 min, 62% B; wavelength: 220 nm; RT1(min): 6.47) to afford N-(3-cyano-4-(3-hydroxyazetidine-1-carbonyl)phenyl)-4-cyclopropyl-3-(quinolin-5-yl)isothiazole-5-carboxamide (I-180, 3.8 mg, 13.69%) as a white solid. LCMS (ES, m/z): [M+1]⁺=496; ¹H NMR (400 MHz, DMSO-d₆) δ 0.25 (d, J=4.0 Hz, 2H), 0.54 (dd, J=2.0, 8.4 Hz, 2H), 1.94 (d, J=5.6 Hz, 1H), 3.80-3.84 (m, 1H), 3.98 (d, J=7.9 Hz, 1H), 4.27-4.36 (m, 2H) 4.51-4.57 (m, 1H), 5.84 (d, J=6.4 Hz, 1H), 7.59 (dd, J=4.4, 8.8 Hz, 1H), 7.76 (d, J=7.2 Hz, 1H), 7.90 (t, J=7.2, 1H), 8.02 (d, J=8.4 Hz, 1H), 8.14 (d, J=8.0 Hz, 1H), 8.19 (d, J=8.4 Hz, 2H), 8.25 (d, J=2.0 Hz, 1H), 8.99 (dd, J=1.6, 4.0 Hz, 1H), 11.15 (s, 1H),

### EXAMPLE 266—SYNTHESIS OF 4-CYCLOPROPYL-3-(1-METHYL-1H-PYRAZOL-4-YL)-N-(2-(TRIFLUOROMETHYL)PYRIDIN-4-YL)ISOTHIAZOLE-5-CARBOXAMIDE (I-181)

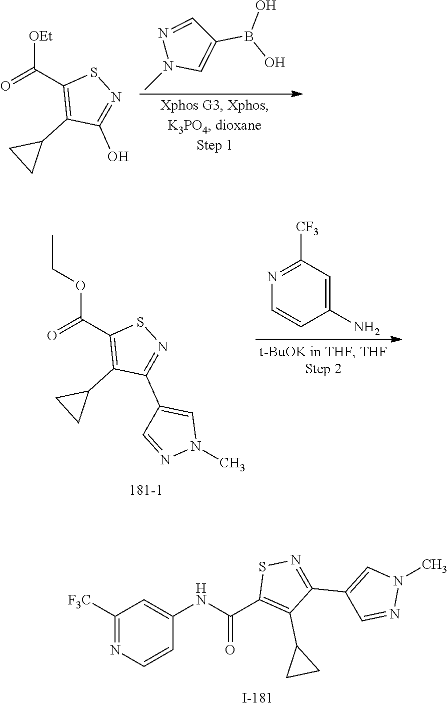

Step 1: Synthesis of 181-1: A solution of ethyl 4-cyclopropyl-3-(trifluoromethanesulfonyloxy)-1,2-thiazole-5-carboxylate (150 mg, 0.434 mmol, 1 equiv), 1-methylpyrazol-4-ylboronic acid (40 mg, 0.032 mmol, 1.47 equiv), XPhos Pd G3 (60 mg, 0.071 mmol, 0.16 equiv), X-Phos (45 mg, 0.094 mmol, 0.22 equiv), and K₃PO₄ (230 mg, 1.084 mmol, 2.49 equiv) in dioxane (10 mL) was stirred overnight at 60° C. under a nitrogen atmosphere. The resulting mixture was diluted with water (10 mL). The resulting mixture was extracted with EtOAc (3×10 mL). The combined organic layers were washed with brine (1×20 mL) and dried over anhydrous Na₂SO₄. After filtration, the filtrate was concentrated under reduced pressure. The residue was purified by prep-TLC (PE/EA 5:1) to afford ethyl 4-cyclopropyl-3-(1-methylpyrazol-4-yl)-1,2-thiazole-5-carboxylate (181-1, 80 mg, 67.13%) as a yellow solid.

Step 2: Synthesis of 4-cyclopropyl-3-(1-methyl-1H-pyrazol-4-yl)-N-(2-(trifluoromethyl)pyridin-4-yl)isothiazole-5-carboxamide (I-181): To a stirred solution of ethyl 4-cyclopropyl-3-(1-methylpyrazol-4-yl)-1,2-thiazole-5-carboxylate (300 mg, 1.082 mmol, 1 equiv) and 2-(trifluoromethyl)pyridin-4-amine (175 mg, 1.082 mmol, 1 equiv) in THF (15 mL) was added t-BuOK (364 mg, 3.246 mmol, 3 equiv) at 0° C. under a nitrogen atmosphere. The resulting mixture was stirred for 20 min at 0° C. under a nitrogen atmosphere. The mixture was acidified to pH 5 with HCl (aq.). The resulting mixture was extracted with EtOAc (3×15 mL). The combined organic layers were washed with brine (1×40 mL) and dried over anhydrous Na₂SO₄. After filtration, the filtrate was concentrated under reduced pressure. The crude product was purified by prep-HPLC with the following conditions: (column: Xselect CSH C18 OBD column 30*150 mm 5 μm; mobile phase A: water (0.1% FA), mobile phase B: ACN; flow rate: 60 mL/min; gradient: 24% B to 40% B in 8 min, 40% B; wavelength: 254; 220 nm; RT1(min): 7.3; injection volume: 1 mL; number of runs: 3) to afford 4-cyclopropyl-3-(1-methylpyrazol-4-yl)-N-[2-(trifluoromethyl)pyridin-4-yl]-1,2-thiazole-5-carboxamide (I-181, 35.3 mg, 8.28%) as an off-white solid. LCMS (ES, m/z): [M+H]⁺=394; ¹H NMR (400 MHz, DMSO-d₆) δ 0.31-0.58 (m, 2H), 0.90-1.17 (m, 2H), 1.91-2.25 (m, 1H), 3.94 (s, 3H), 7.93 (dd, J=8.0, 1.6 Hz, 1H), 8.05 (s, 1H), 8.20 (d, J=1.6 Hz, 1H), 8.39 (s, 1H), 8.72 (d, J=5.6 Hz, 1H), 11.40 (s, 1H).

### EXAMPLE 267—SYNTHESIS OF 4-CYCLOPROPYL-3-(1H-INDAZOL-4-YL)-N-(2-(TRIFLUOROMETHYL)PYRIDIN-4-YL)ISOTHIAZOLE-5-CARBOXAMIDE (I-182)

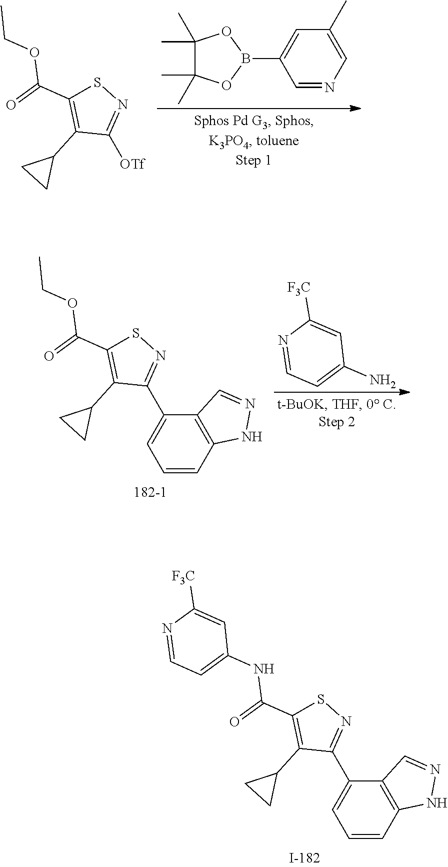

Step 1: Synthesis of 182-1: To a stirred solution of ethyl 4-cyclopropyl-3-(trifluoromethanesulfonyloxy)-1,2-thiazole-5-carboxylate (200 mg, 0.579 mmol, 1 equiv) and 4-(4,4,5,5-tetramethyl-1,3,2-dioxaborolan-2-yl)-1H-indazole (141 mg, 0.579 mmol, 1 equiv) in toluene (3 mL) and H₂O (0.6 mL) was added K₃PO₄ (246 mg, 1.158 mmol, 2 equiv) and SPhos Pd G3 (45 mg, 0.058 mmol, 0.1 equiv). The resulting mixture was stirred for 16 h at 90° C. under a nitrogen atmosphere. The resulting mixture was diluted with water (20 mL). The resulting mixture was extracted with EtOAc (3×20 mL). The combined organic layers were washed with brine (1×50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% TFA); 10% to 50% gradient in 10 min; detector: UV 254 nm to afford ethyl 4-cyclopropyl-3-(1H-indazol-4-yl)-1,2-thiazole-5-carboxylate (182-1, 100 mg, 55.09%) as an off-white solid. LCMS (ES, m/z): [M+1]⁺=314.

Step 2: Synthesis of 4-cyclopropyl-3-(1H-indazol-4-yl)-N-(2-(trifluoromethyl)pyridin-4-yl)isothiazole-5-carboxamide (I-182): A solution of ethyl 4-cyclopropyl-3-(1H-indazol-4-yl)-1,2-thiazole-5-carboxylate (100 mg, 0.319 mmol, 1 equiv) and 2-(trifluoromethyl)pyridin-4-amine (52 mg, 0.319 mmol, 1 equiv) in THF (4 mL) was treated with t-BuOK (72 mg, 0.638 mmol, 2 equiv) at 0° C. The resulting mixture was stirred for 1 h at room temperature. The resulting mixture was diluted with water (20 mL). The resulting mixture was extracted with EtOAc (3×20 mL). The combined organic layers were washed with brine (1×50 mL) and dried over anhydrous Na₂SO₄. After filtration, the filtrate was concentrated under reduced pressure. The crude product (100 mg) was purified by prep-HPLC with the following conditions: (column: XBridge Prep OBD C18 column: 30*150 mm, 5 μm; mobile phase A: water (10 mmol/L NH₄HCO₃), mobile phase B: ACN; flow rate: 60 mL/min; gradient: 36% B to 62% B in 8 min, 62% B; wavelength: 254 nm; RT1(min): 7.62; injection volume: 0.3 mL; number of runs: 5) to afford 4-cyclopropyl-3-(1H-indazol-4-yl)-N-[2-(trifluoromethyl)pyridin-4-yl]-1,2-thiazole-5-carboxamide (I-182, 36.3 mg, 26.44%) as a white solid. LCMS (ES, m/z): [M+1]⁺=430; ¹H NMR (400 MHz, DMSO-d₆) δ 0.28-0.34 (m, 2H), 0.69-0.82 (m, 2H), 2.24-2.28 (m, 1H), 6.49 (s, 1H), 7.50 (t, J=8.0 Hz, 1H), 7.58 (d, J=6.8 Hz, 1H), 7.68 (d, J=8.0 Hz, 1H), 7.90 (d, J=5.2 Hz, 1H), 8.21 (s, 1H), 8.22 (d, J=2.8 Hz, 1H), 8.67 (d, 0.1=5.2 Hz, 1H), 13.27 (s, 1H).

### EXAMPLE 268—SYNTHESIS OF 4-CYCLOPROPYL-3-(1,5-DIMETHYL-1H-PYRAZOL-4-YL)-N-(2-(TRIFLUOROMETHYL)PYRIDIN-4-YL)ISOTHIAZOLE-5-CARBOXAMIDE (I-183)

Step 1: Synthesis of 183-1: A mixture of ethyl 4-cyclopropyl-3-(((trifluoromethyl)sulfonyl)oxy)isothiazole-5-carboxylate (150.00 mg, 0.434 mmol, 1 equiv), 1,5-dimethyl-4-(4,4,5,5-tetramethyl-1,3,2-dioxaborolan-2-yl)-1H-pyrazole (115.77 mg, 0.521 mmol, 1.2 equiv), X-Phos (51.77 mg, 0.108 mmol, 0.25 equiv), XPhos Pd G3 (73.54 mg, 0.087 mmol, 0.2 equiv) and K₃PO₄ (276.62 mg, 1.302 mmol, 3 equiv) in dioxane (4 mL) was stirred for 2 h at 60° C. under a nitrogen atmosphere. The resulting mixture was diluted with water (10 mL) and extracted with EtOAc (3×15 mL). The combined organic layers were washed with brine (1×40 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 40% to 50% gradient in 10 min; detector: UV 254 nm, to afford ethyl 4-cyclopropyl-3-(1,5-dimethyl-1H-pyrazol-4-yl)isothiazole-5-carboxylate (183-1, 70.00 mg, 54.31%) as an off-white solid.

Step 2: Synthesis of 4-cyclopropyl-3-(1,5-dimethyl-1H-pyrazol-4-yl)-N-(2-(trifluoromethyl)pyridin-4-yl)isothiazole-5-carboxamide (I-183): To a stirred mixture of ethyl 4-cyclopropyl-3-(1,5-dimethyl-1H-pyrazol-4-yl)isothiazole-5-carboxylate (80.00 mg, 0.275 mmol, 1 equiv) and 2-(trifluoromethyl)pyridin-4-amine (53.41 mg, 0.330 mmol, 1.2 equiv) in THF (3 mL) was added t-BuOK (92.43 mg, 0.825 mmol, 3 equiv) at room temperature. The resulting mixture was stirred for 30 min at room temperature. The resulting mixture was diluted with water (10 mL) and extracted with EtOAc (3×15 mL). The combined organic layers were washed with brine (1×40 mL) and dried over anhydrous Na₂SO₄. After filtration, the filtrate was concentrated under reduced pressure. The crude product was purified by prep-HPLC with the following conditions: column: XBridge Prep OBD C18 column: 30*150 mm, 5 μm; flow rate: 60 mL/min; gradient: 33% B to 58% B in 8 min, 58% B; wavelength: 254 nm; RT1(min): 7.50; injection volume: 0.7 mL, to afford 4-cyclopropyl-3-(1,5-dimethyl-1H-pyrazol-4-yl)-N-(2-(trifluoromethyl)pyridin-4-yl)isothiazole-5-carboxamide (I-183, 36.2 mg, 32.26%) as an off-white solid. LCMS (ES, m/z): [M+H]⁺=408; ¹H NMR (400 MHz, DMSO-d₆) δ 0.35-0.44 (m, 2H), 0.83-0.98 (m, 2H), 2.10-2.21 (m, 1H), 2.42 (s, 3H), 3.82 (s, 3H), 7.97 (dd, J=1.2, 5.2 Hz, 1H), 7.95 (s, 1H), 8.20 (d, J=2.0 Hz, 1H), 8.71 (d, J=4.0 Hz, 1H), 11.33 (s, 1H).

### EXAMPLE 268—SYNTHESIS OF 4-CYCLOPROPYL-3-(PYRIDIN-4-YL)-N-(2-(TRIFLUOROMETHYL)PYRIDIN-4-YL)ISOTHIAZOLE-5-CARBOXAMIDE (I-184)

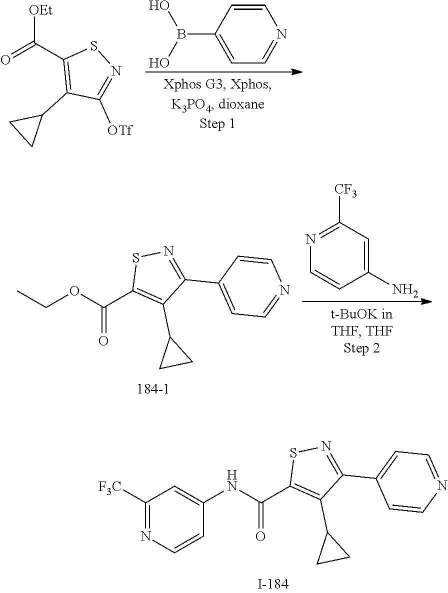

Step 1: Synthesis of 184-1: A solution of ethyl 4-cyclopropyl-3-(trifluoromethanesulfonyloxy)-1,2-thiazole-5-carboxylate (150 mg, 0.434 mmol, 1 equiv), pyridin-4-ylboronic acid (60 mg, 0.488 mmol, 1.12 equiv), XPhos Pd G3 (60 mg, 0.071 mmol, 0.16 equiv), X-Phos (45 mg, 0.094 mmol, 0.22 equiv), and K₃PO₄ (230 mg, 1.084 mmol, 2.49 equiv) in dioxane (10 mL) was stirred overnight at 60° C. under a nitrogen atmosphere. The resulting mixture was diluted with water (10 mL). The resulting mixture was extracted with EtOAc (3×10 mL). The combined organic layers were washed with brine (1×20 mL) and dried over anhydrous Na₂SO₄. After filtration, the filtrate was concentrated under reduced pressure. The residue was purified by prep-TLC (PE/EA 5:1) to afford ethyl 4-cyclopropyl-3-(pyridin-4-yl)-1,2-thiazole-5-carboxylate (184-1, 80 mg, 67.13%) as a yellow solid.

Step 2: Synthesis of 4-cyclopropyl-3-(pyridin-4-yl)-N-(2-(trifluoromethyl)pyridin-4-yl)isothiazole-5-carboxamide (I-184): A solution of ethyl 4-cyclopropyl-3-(pyridin-3-yl)-1,2-thiazole-5-carboxylate (80 mg, 0.292 mmol, 1 equiv) and 2-(trifluoromethyl)pyridin-4-amine (57 mg, 0.352 mmol, 1.21 equiv) and t-BuOK (100 mg, 0.891 mmol, 3.06 equiv) in THF (5 mL) was stirred for 0.5 h at room temperature under a nitrogen atmosphere. The resulting mixture was diluted with water (5 mL). The resulting mixture was extracted with EtOAc (3×10 mL). The combined organic layers were washed with brine (1×20 mL) and dried over anhydrous Na₂SO₄. After filtration, the filtrate was concentrated under reduced pressure. The crude product (50 mg) was purified by prep-HPLC with the following conditions: (column: Xselect CSH C18 OBD column 30*150 mm 5 μm; mobile phase A: water (0.1% FA), mobile phase B: ACN; flow rate: 60 mL/min; gradient: 31% B to 41% B in 7 min, 41% B; wavelength: 254; 220 nm; RT1(min): 5.65, 8.32) to afford 4-cyclopropyl-3-(pyridin-4-yl)-N-[2-(trifluoromethyl)pyridin-4-yl]-1,2-thiazole-5-carboxamide (I-184, 8.7 mg, 7.62%) as an off-white solid. LCMS (ES, m/z): [M+H]⁺=391; ¹H NMR (400 MHz, DMSO-d₆) δ δ 0.21-0.48 (m, 2H), 0.75-1.01 (m, 2H), 2.20-2.30 (m, 1H), 7.85 (dd, J=4.4, 1.6 Hz, 1H), 7.94 (dd, J=5.6, 1.6 Hz, 1H), 8.22 (d, J=1.6 Hz, 1H), 8.60-8.89 (m, 3H), 11.40 (s, 1H).

### EXAMPLE 269—SYNTHESIS OF 4-CYCLOPROPYL-3-(IMIDAZO[1,2-A]PYRIDIN-7-YL)-N-(2-(TRIFLUOROMETHYL)PYRIDIN-4-YL)ISOTHIAZOLE-5-CARBOXAMIDE (I-185)

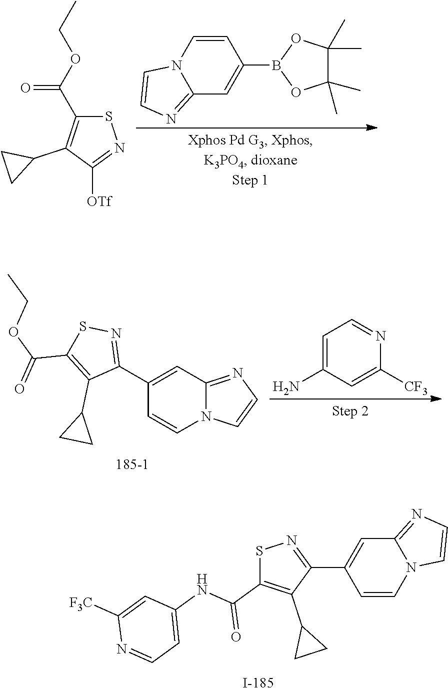

Step 1: Synthesis of 185-1: A solution of ethyl 4-cyclopropyl-3-(trifluoromethanesulfonyloxy)-1,2-thiazole-5-carboxylate (150 mg, 0.434 mmol, 1 equiv), 7-(4,4,5,5-tetramethyl-1,3,2-dioxaborolan-2-yl)imidazo[1,2-a]pyridine (127 mg, 0.521 mmol, 1.2 equiv), XPhos Pd G3 (74 mg, 0.087 mmol, 0.2 equiv), XPhos (52 mg, 0.108 mmol, 0.25 equiv) and K₃PO₄ (277 mg, 1.302 mmol, 3 equiv) in dioxane (2 mL) was stirred for 2 h at 60° C. under a nitrogen atmosphere. The resulting mixture was diluted with water (10 mL). The resulting mixture was extracted with EtOAc (3×10 mL). The combined organic layers were washed with brine (1×30 mL) and dried over anhydrous Na₂SO₄. After filtration, the filtrate was concentrated under reduced pressure. The residue was purified by silica gel column chromatography and eluted with PE/EA (5:1) to afford ethyl 4-cyclopropyl-3-{imidazo[1,2-a]pyridin-7-yl}-1,2-thiazole-5-carboxylate (185-1, 60 mg, 13.22%) as a light yellow oil. LCMS (ES, m/z): [M+H]⁺ =314.

Step 2: Synthesis of 4-cyclopropyl-3-(imidazo[1,2-a]pyridin-7-yl)-N-(2-(trifluoromethyl)pyridin-4-yl)isothiazole-5-carboxamide (I-185): To a stirred solution of ethyl 4-cyclopropyl-3-{imidazo[1,2-a]pyridin-7-yl}-1,2-thiazole-5-carboxylate (60 mg, 0.191 mmol, 1 equiv) and 2-(trifluoromethyl)pyridin-4-amine (37.2 mg, 0.229 mmol, 1.2 equiv) in THF (2 mL) was added t-BuOK (64.4 mg, 0.573 mmol, 3 equiv) at 0° C. under a nitrogen atmosphere. The resulting mixture was stirred for 2 h at room temperature under a nitrogen atmosphere. The resulting mixture was diluted with water (10 mL). The resulting mixture was extracted with EtOAc (3×10 mL). The combined organic layers were washed with brine (30 mL) and dried over anhydrous Na₂SO₄. After filtration, the filtrate was concentrated under reduced pressure. The crude product (40 mg) was purified by prep-HPLC with the following conditions: column: XBridge Prep OBD C18 column: 30*150 mm, 5 m; mobile phase A: water (10 mmol/L NH₄HCO₃), mobile phase B: ACN; flow rate: 60 mL/min; gradient: 37% B to 55% B in 8 min, 55% B; wavelength: 254 nm; RT1(min): 7.3 to afford 4-cyclopropyl-3-{imidazo[1,2-a]pyridin-7-yl}-N-[2-(trifluoromethyl)pyridin-4-yl]-1,2-thiazole-5-carboxamide (I-185, 1.1 mg, 1.33%) as an off-white solid. LCMS (ES, m % z): [M+H]⁺=430; ¹H NMR (400 MHz, DMSO-d₆) δ 0.35-0.45 (m, 2H), 0.85-0.98 (m, 2H), 2.31-2.39 (m, 1H), 7.43 (dd, J=1.6, 7.2 Hz, 1H), 7.70 (s, 1H), 7.94 (d, J=5.2 Hz, 1H), 8.05 (s, 1H), 8.07-8.12 (d, J=8.8 Hz, 1H), 8.23 (d, J=1.6 Hz, 1H), 8.6 (d, J=6.4 Hz, 1H), 8.73 (d, J=5.6 Hz, 1H), I 1.41 (s, 1H).

### EXAMPLE 270—SYNTHESIS OF 4-CYCLOPROPYL-3-(IMIDAZO[1,2-A]PYRIDIN-7-YL)-N-(2-(TRIFLUOROMETHYL)PYRIDIN-4-YL)ISOTHIAZOLE-5-CARBOXAMIDE (I-186)

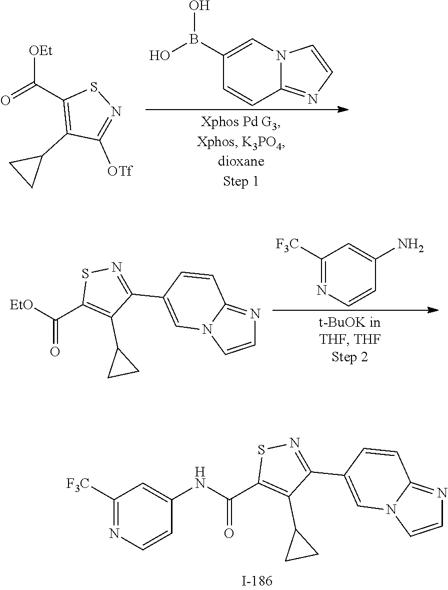 [SMILES: CCOC(=O)c1snc(OS(=O)(=O)C(F)(F)F)c1C1CC1 ; OB(O)c1ccc2nccn2c1 ; CCOC(=O)c1snc(-c2ccc3nccn3c2)c1C1CC1 ; Nc1ccnc(C(F)(F)F)c1 ; O=C(Nc1ccnc(C(F)(F)F)c1)c1snc(-c2ccc3nccn3c2)c1C1CC1]

Step 1: Synthesis of 186-1: A solution of ethyl 4-cyclopropyl-3-(trifluoromethanesulfonyloxy)-1,2-thiazole-5-carboxylate (150 mg, 0.434 mmol, 1 equiv), imidazo[1,2-a]pyridin-6-ylboronic acid (84 mg, 0.519 mmol, 1.19 equiv), XPhos (42 mg, 0.088 mmol, 0.20 equiv), XPhos Pd G₃ (74 mg, 0.087 mmol, 0.20 equiv) and K₃PO₄ (276 mg, 1.300 mmol, 2.99 equiv) in 1,4-dioxane (10 mL) was stirred overnight at 60° C. under a nitrogen atmosphere. The resulting mixture was diluted with water (10 mL). The resulting mixture was extracted with EtOAc (3×20 mL). The combined organic layers were washed with brine (lx 40 mL) and dried over anhydrous Na₂SO₄. After filtration, the filtrate was concentrated under reduced pressure. The crude product (80 mg) was purified by prep-HPLC with the following conditions: (column: XBridge Prep OBD C18 column: 30*150 mm, 5 μm; mobile phase A: water (10 mmol/L NH₄HCO₃+0.1% NH₃·H₂O), mobile phase B: ACN; flow rate: 60 mL/min; gradient: 36% B to 65% B in 8 min, 65% B; wavelength: 220 nm; RT1(min): 7.47; number of runs: 1) to afford ethyl 4-cyclopropyl-3-{imidazo[1,2-a]pyridin-6-yl}-1,2-thiazole-5-carboxylate (186-1, 80 mg, 58.77%) as a light yellow oil.

Step 2: Synthesis of 4-cyclopropyl-3-(imidazo[1,2-a]pyridin-7-yl)-N-(2-(trifluoromethyl)pyridin-4-yl)isothiazole-5-carboxamide (I-186): A solution of ethyl 4-cyclopropyl-3-{imidazo[1,2-a]pyridin-6-yl}-1,2-thiazole-5-carboxylate (80 mg, 0.255 mmol, 1 equiv), 2-(trifluoromethyl)pyridin-4-amine (45 mg, 0.278 mmol, 1.09 equiv) and t-BuOK (86 mg, 0.766 mmol, 3.00 equiv) in THF (5 mL) was stirred for 0.5 h at room temperature under a nitrogen atmosphere. The resulting mixture was diluted with water (5 mL). The resulting mixture was extracted with EtOAc (3×10 mL). The combined organic layers were washed with brine (1×20 mL) and dried over anhydrous Na₂SO₄. After filtration, the filtrate was concentrated under reduced pressure. The crude product (50 mg) was purified by prep-HPLC with the following conditions: (column: XBridgePrep OBD C18 column: 30*150 mm, 5 μm; flow rate: 60 mL/min; gradient: 30% B to 55% B in 8 min, 55% B; wavelength: 254 nm; RT1(min): 7.73; injection volume: 0.4 mL; number of runs: 5) to afford 4-cyclopropyl-3-{imidazo[1,2-a]pyridin-6-yl}-N-[2-(trifluoromethyl)pyridin-4-yl]-1,2-thiazole-5-carboxamide (I-186, 4.2 mg, 3.72%) as an off-white solid. LCMS (ES, m/z): [M+1]⁺=430; ¹H NMR (400 MHz, DMSO-d₆) δ 0.27-0.58 (m, 2H), 0.80-1.09 (m, 2H), 2.21-2.35 (m, 1H), 7.60-7.82 (m, 3H), 7.94 (d, J=4.8 Hz, 1H), 8.13 (s, 1H), 8.24 (d, J=2.0 Hz, 1H), 8.73 (d, J=5.2 Hz, 1H), 9.18 (s, 1H), 11.40 (s, 1H).

### EXAMPLE 271—SYNTHESIS OF 4-CYCLOPROPYL-3-(3,5-DICHLOROPYRIDIN-4-YL)-N-(2-(TRIFLUOROMETHYL)PYRIDIN-4-YL)ISOTHIAZOLE-5-CARBOXAMIDE (I-187)

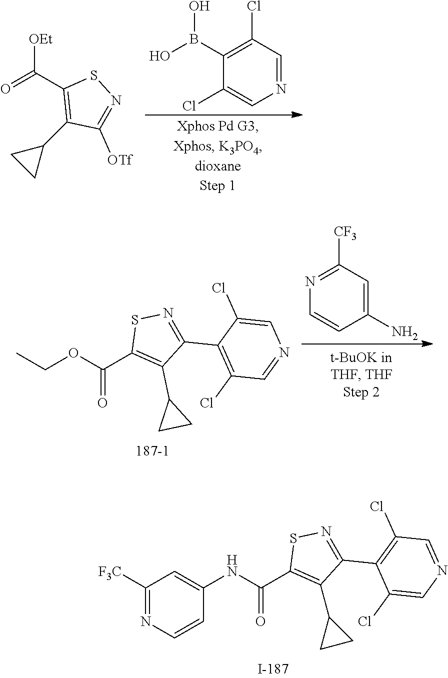

Step 1: Synthesis of 187-1: A solution of ethyl 4-cyclopropyl-3-(trifluoromethanesulfonyloxy)-1,2-thiazole-5-carboxylate (150 mg, 0.434 mmol, 1 equiv), 3,5-dichloropyridin-4-ylboronic acid (91 mg, 0.474 mmol, 1.09 equiv), XPhos (42 mg, 0.088 mmol, 0.20 equiv), XPhos Pd G3 (74 mg, 0.087 mmol, 0.20 equiv) and K₃PO₄ (276 mg, 1.300 mmol, 2.99 equiv) in 1,4-dioxane (10 mL) was stirred overnight at 60° C. under a nitrogen atmosphere. The resulting mixture was diluted with water (10 mL). The resulting mixture was extracted with EtOAc (3×20 mL). The combined organic layers were washed with brine (1×40 mL) and dried over anhydrous Na₂SO₄. After filtration, the filtrate was concentrated under reduced pressure. The crude product (80 mg) was purified by prep-HPLC with the following conditions: (column: XBridge Prep OBD C18 column: 30*150 mm, 5 μm; mobile phase A: water (10 mmol/L NH₄HCO₃+0.1% NH₃·H₂O), mobile phase B: ACN; flow rate: 60 mL/min; gradient: 36% B to 65% B in 8 min, 65% B; wavelength: 220 nm; RT1(min): 7.47; number of runs: 1) to afford ethyl 4-cyclopropyl-3-(3,5-dichloropyridin-4-yl)-1.2-thiazole-5-carboxylate (187-1, 80 mg, 53.66%) as a light yellow oil.

Step 2: Synthesis of 4-cyclopropyl-3-(3,5-dichloropyridin-4-yl)-N-(2-(trifluoromethyl)pyridin-4-yl)isothiazole-5-carboxamide (1-187): A solution of ethyl 4-cyclopropyl-3-(3,5-dichloropyridin-4-yl)-1,2-thiazole-5-carboxylate (80 mg, 0.233 mmol, 1 equiv), 2-(trifluoromethyl)pyridin-4-amine (42 mg, 0.259 mmol, 1.11 equiv) and t-BuOK (78 mg, 0.695 mmol, 2.98 equiv) in THF (5 mL) was stirred for 0.5 h at room temperature under a nitrogen atmosphere. The resulting mixture was diluted with water (5 mL). The resulting mixture was extracted with EtOAc (3×10 mL). The combined organic layers were washed with brine (1×4 mL) and dried over anhydrous Na₂SO₄. After filtration, the filtrate was concentrated under reduced pressure. The crude product (50 mg) was purified by prep-HPLC with the following conditions: (column: XBridge Prep OBD C18 column: 30*150 mm, 5 μm; flow rate: 60 m/min; gradient: 30% B to 55% B in 8 min, 55% B; wavelength: 254 nm; RT1(min): 7.73; injection volume: 0.4 mL; number of runs: 5) to afford 4-cyclopropyl-3-(3,5-dichloropyridin-4-yl)-N-[2-(trifluoromethyl)pyridin-4-yl]-1,2-thiazole-5-carboxamide (I-187, 3.5 mg, 3.27%) as an off-white solid. LCMS (ES, m/z): [M+1]⁺=459; ¹H NMR (400 MHz, DMSO-d₆) δ 0.11-0.48 (m, 2H), 0.51-0.81 (m, 2H), 1.75-2.02 (m, 1H), 7.98 (dd, J=5.6, 2.0 Hz, 1H), 8.23 (d, J=2.0 Hz, 1H), 8.73 (d, J=5.6 Hz, 1H), 8.90 (s, 2H), 11.42 (s, 1H).

### EXAMPLE 272—SYNTHESIS OF N-(5-CYANO-6-(2H-1,2,3-TRIAZOL-2-YL)PYRIDIN-3-YL)-4-CYCLOPROPYL-3-(1-OXO-1,2-DIHYDROISOQUINOLIN-5-YL) ISOTHIAZOLE-5-CARBOXAMIDE (I-188)

Step 1: Synthesis of 188-1: To a stirred mixture of 1-methoxy-5-(4,4,5,5-tetramethyl-1,3,2-dioxaborolan-2-yl)isoquinoline (825.78 mg, 2.896 mmol, 1.00 equiv) and ethyl 4-cyclopropyl-3-(trifluoromethanesulfonyloxy)-1,2-thiazole-5-carboxylate (1 g, 2.896 mmol, 1.00 equiv) in toluene (10 mL) was added K₃PO₄ (1844.13 mg, 8.688 mmol, 3.00 equiv), XPhos (276.12 mg, 0.579 mmol, 0.2 equiv) and XPhos Pd G3 (245.13 mg, 0.290 mmol, 0.1 equiv) in portions at room temperature under a nitrogen atmosphere. The resulting mixture was stirred for an additional 3 h at 60° C. The resulting mixture was diluted with water (30 mL). The resulting mixture was extracted with EtOAc (3×50 mL). The combined organic layers were washed with brine (1×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄HCO₃), 10% to 50% gradient in 10 min; detector: UV 254 nm. This resulted in ethyl 4-cyclopropyl-3-(1-methoxyisoquinolin-5-yl)-1,2-thiazole-5-carboxylate (188-1, 360 mg, 31.57%) as a yellow oil.

Step 2: Synthesis of 188-2: To a stirred mixture of ethyl 4-cyclopropyl-3-(1-methoxyisoquinolin-5-yl)-1,2-thiazole-5-carboxylate (100 mg, 0.282 mmol, 1 equiv) and 5-amino-2-(1,2,3-triazol-2-yl)pyridine-3-carbonitrile (52.53 mg, 0.282 mmol, 1 equiv) in THF (2 mL) was added t-BuOK (63.32 mg, 0.564 mmol, 2 equiv) in portions at room temperature under a nitrogen atmosphere. The resulting mixture was stirred for an additional 0.5 h at room temperature. The resulting mixture was diluted with water (20 mL). The resulting mixture was extracted with EtOAc (2×50 mL). The combined organic layers were washed with brine (1×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄HCO₃), 10% to 50% gradient in 10 min; detector: UV 254 nm. This resulted in N-[5-cyano-6-(1,2,3-triazol-2-yl)pyridin-3-yl]-4-cyclopropyl-3-(1-methoxyisoquinolin-5-yl)-1,2-thiazole-5-carboxamide (188-2, 50 mg, 32.25%) as a white solid.

Step 3: Synthesis of N-(5-cyano-6-(2H-1,2,3-triazol-2-yl)pyridin-3-yl)-4-cyclopropyl-3-(1-oxo-1,2-dihydroisoquinolin-5-yl)isothiazole-5-carboxamide (I-188): To a stirred mixture of N-[5-cyano-6-(1,2,3-triazol-2-yl)pyridin-3-yl]-4-cyclopropyl-3-(1-methoxyisoquinolin-5-yl)-1,2-thiazole-5-carboxamide (20 mg, 0.040 mmol, 1 equiv) in HOAc (1 mL) was added HBr (9.82 mg, 0.120 mmol, 3 equiv) in portions at room temperature under a nitrogen atmosphere. The resulting mixture was stirred for an additional 3 h at 50° C. The resulting mixture was diluted with water (10 mL). The resulting mixture was extracted with EtOAc (3×30 mL). The combined organic layers were washed with brine (1×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄HCO₃), 10% to 50% gradient in 10 min; detector: UV 254 nm. This resulted in N-[5-cyano-6-(1,2,3-triazol-2-yl)pyridin-3-yl]-4-cyclopropyl-3-(1-oxo-2H-isoquinolin-5-yl)-1,2-thiazole-5-carboxamide (I-188, 4.1 mg, 20.99%) as a white solid. LCMS (ES, m/z): [M+H]⁺=481; ¹H NMR (400 MHz, DMSO-d₆): δ 0.26-0.34 (m, 2H), 0.58-0.67 (m, 2H), 1.95 (t, J=13.6 Hz, 1H), 6.22 (d, J=7.4 Hz, 1H), 7.21 (dd, J=7.4, 5.8 Hz, 1H), 7.63 (t, J=7.8 Hz, 1H), 7.79 (dd, J=7.4, 1.4 Hz, 1H), 8.32 (s, 2H), 8.40-8.34 (m, 1H), 8.86 (d, J=2.6 Hz, 1H), 9.08 (d, J=2.6 Hz, 1H), 11.40 (s, 1H), 11.45 (d, J=5.8 Hz, 1H).

### EXAMPLE 273—SYNTHESIS OF 4-CYCLOPROPYL-3-(1-OXO-1,2-DIHYDRO-ISOQUINOLIN-5-YL)-N-(2-(TRIFLUOROMETHYL)PYRIDIN-4-YL)ISOTHIAZOLE-5-CARBOXAMIDE (1-189)

Step 1: Synthesis of 189-1: To a stirred mixture of ethyl 4-cyclopropyl-3-(1-methoxyisoquinolin-5-yl)-1,2-thiazole-5-carboxylate (100 mg, 0.282 mmol, 1 equiv) and 2-(trifluoromethyl)pyridin-4-amine (45.74 mg, 0.282 mmol, 1 equiv) in THF (2 mL) was added t-BuOK (94.98 mg, 0.846 mmol, 3 equiv) in portions at room temperature under a nitrogen atmosphere. The resulting mixture was stirred for an additional 0.5 h at room temperature. The resulting mixture was diluted with water (10 mL). The resulting mixture was extracted with EtOAc (3×30 mL). The combined organic layers were washed with brine (1×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄HCO₃), 10% to 50% gradient in 10 min; detector: UV 254 nm. This resulted in 4-cyclopropyl-3-(1-methoxyisoquinolin-5-yl)-N-[2-(trifluoromethyl)pyridin-4-yl]-1,2-thiazole-5-carboxamide (189-1, 70 mg, 47.46%) as a white solid.

Step 2: Synthesis of 4-cyclopropyl-3-(1-oxo-1,2-dihydroisoquinolin-5-yl)-N-(2-(trifluoromethyl)pyridin-4-yl)isothiazole-5-carboxamide (1-189): To a stirred mixture of 4-cyclopropyl-3-(1-methoxyisoquinolin-5-yl)-N-[2-(trifluoromethyl)pyridin-4-yl]-1,2-thiazole-5-carboxamide (50 mg, 0.106 mmol, 1 equiv) in HOAc (2 mL) was added HBr (25.80 mg, 0.318 mmol, 3 equiv) in portions at room temperature under a nitrogen atmosphere. The resulting mixture was stirred for an additional 3 h at 50° C. The resulting mixture was diluted with water (10 mL). The resulting mixture was extracted with EtOAc (3×30 mL). The combined organic layers were washed with brine (1×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (10 mmol/L NH₄HCO₃), 10% to 50% gradient in 10 min; detector: UV 254 nm. This resulted in 4-cyclopropyl-3-(1-oxo-2H-isoquinolin-5-yl)-N-[2-(trifluoromethyl)pyridin-4-yl]-1,2-thiazole-5-carboxamide (I-189, 19.3 mg, 39.15%) as a white solid. LCMS (ES, m/z): [M+H]⁺=457; ¹H NMR (400 MHz, CDCl₃) δ 0.49 (q, J=5.4 Hz, 2H), 0.93 (d, J=7.8 Hz, 2H), 2.01 (m, J=4.4 Hz, 1H), 6.53 (d, J=7.4 Hz, 1H), 7.16 (s, 1H), 7.67 (t, J=7.8 Hz, 1H), 7.82-7.75 (m, 1H), 7.86 (d, J=5.6 Hz, 1H), 8.01 (d, J=1.8 Hz, 1H), 8.60 (d, J=8.0 Hz, 1H), 8.74 (d, J=5.6 Hz, 1H), 9.13 (s, 1H), 10.09 (s, 1H).

### EXAMPLE 274—SYNTHESIS OF N-(3-CHLORO-4-(3-HYDROXYAZETIDINE-1-CARBONYL)PHENYL)-4-CYCLOPROPYL-3-(IMIDAZO[1,2-A]PYRIDIN-5-YL) ISOTHIAZOLE-5-CARBOXAMIDE (I-190)

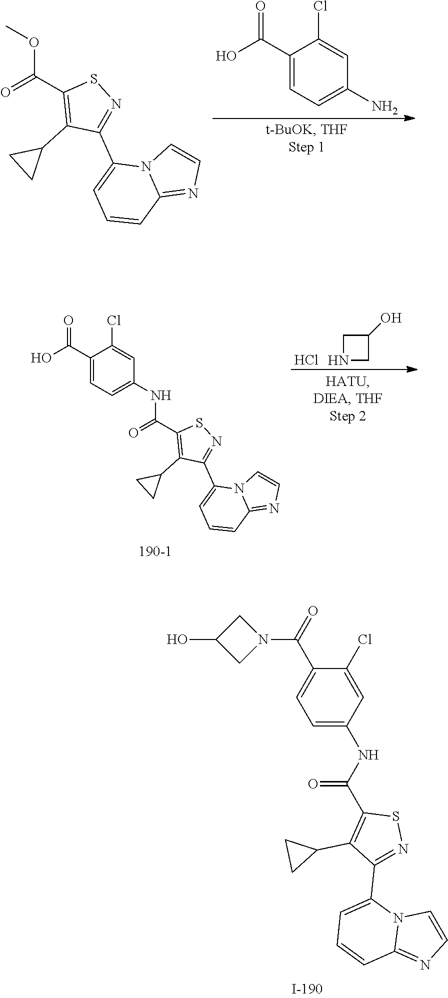 [SMILES: COC(=O)c1snc(-c2cccc3nccn23)c1C1CC1 ; Nc1ccc(C(=O)O)c(Cl)c1 ; O=C(O)c1ccc(NC(=O)c2snc(-c3cccc4nccn34)c2C2CC2)cc1Cl ; Cl ; OC1CNC1 ; O=C(Nc1ccc(C(=O)N2CC(O)C2)c(Cl)c1)c1snc(-c2cccc3nccn23)c1C1CC1]

Step 1: Synthesis of 190-1: A solution of methyl 4-cyclopropyl-3-{imidazo[1,2-a]pyridin-5-yl}-1,2-thiazole-5-carboxylate (150 mg, 0.501 mmol, 1 equiv), 4-amino-2-chlorobenzoic acid (103 mg, 0.600 mmol, 1.20 equiv) and t-BuOK (1.2 mL, 0.011 mmol, 0.02 equiv) in THF (5 mL) was stirred for 0.5 h at room temperature under a nitrogen atmosphere. The resulting mixture was diluted with water (10 mL). The resulting mixture was extracted with EtOAc (3×10 mL). The combined organic layers were washed with brine (1×20 mL) and dried over anhydrous Na₂SO₄. After filtration, the filtrate was concentrated under reduced pressure. The crude product (50 mg) was purified by prep-HPLC with the following conditions: (column: Xselect CSH C18 OBD column 30*150 mm 5 μm; mobile phase A: water (0.1% FA), mobile phase B: ACN; flow rate: 60 mL/min; gradient: 31% B to 41% B in 7 min, 41% B; wavelength: 254; 220 nm; RT1(min): 5.65, 8.32(min)) to afford 2-chloro-4-(4-cyclopropyl-3-{imidazo[1,2-a]pyridin-5-yl}-1,2-thiazole-5-amido)benzoic acid (190-1, 80 mg, 36.38%) as a colorless oil.

Step 2: Synthesis of N-(3-chloro-4-(3-hydroxyazetidine-1-carbonyl)phenyl)-4-cyclopropyl-3-(imidazo[1,2-a]pyridin-5-yl)isothiazole-5-carboxamide (I-190): A solution of 2-chloro-4-(4-cyclopropyl-3-{imidazo[1,2-a]pyridin-5-yl}-1,2-thiazole-5-amido)benzoic acid (70 mg, 0.159 mmol, 1 equiv), azetidin-3-ol hydrochloride (55 mg, 0.502 mmol, 3.15 equiv), DIEA (62 mg, 0.480 mmol, 3.01 equiv) and HATU (100 mg, 0.263 mmol, 1.65 equiv) in THF (5 mL) was stirred overnight at room temperature under a nitrogen atmosphere. The resulting mixture was diluted with water (10 mL). The resulting mixture was extracted with EtOAc (3×20 mL). The combined organic layers were washed with brine (1×30 mL) and dried over anhydrous Na₂SO₄. After filtration, the filtrate was concentrated under reduced pressure. The crude product (50 mg) was purified by prep-HPLC with the following conditions: (column: XBridge Shield RP18 OBD column: 30*150 mm, 5 μm; mobile phase A: water (10 mmol/L NH₄HCO₃+0.1% NH₃·H₂O), mobile phase B: ACN; flow rate: 60 mL/min; gradient: 25% B to 45% B in 8 min, 45% B; wavelength: 220/254 nm; RT1(min): 7.57; injection volume: 2 mL; number of runs: 2) to afford N-[3-chloro-4-(3-hydroxyazetidine-1-carbonyl)phenyl]-4-cyclopropyl-3-{imidazo[1,2-a]pyridin-5-yl}-1,2-thiazole-5-carboxamide (I-190, 2.8 mg, 3.55%) as an off-white solid. LCMS (ES, m/z): [M+1]⁺=494; ¹H NMR (400 MHz, DMSO-d₆) δ 0.47-0.65 (m, 2H), 0.98-1.17 (m, 2H), 2.22-2.38 (m, 1H), 3.68-3.90 (m, 2H), 4.07 (t, J=7.8 Hz, 1H), 4.24 (dd, J=10.4, 7.0 Hz, 1H), 4.42-4.62 (m, 1H), 5.82 (d, 1=6.0 Hz, 1H), 7.18 (t, J=6.8, 1.3 Hz, 1H), 7.44-7.48 (m, 1H), 7.49-7.53 (m, 1H), 7.69 (dd, J=8.4, 2.0 Hz, 1H), 7.77-7.82 (m, 1H), 7.95 (d, J=2.0 Hz, 1H), 8.54 (s, 1H), 9.67 (d, J=7.2, 1.2 Hz, 1H), 11.02 (s, 1H).

### EXAMPLE 275—SYNTHESIS OF 4-CYCLOPROPYL-3-(2-METHYLPYRIDIN-4-YL)-N-(2-(TRIFLUOROMETHYL)PYRIDIN-4-YL)ISOTHIAZOLE-5-CARBOXAMIDE (I-191)

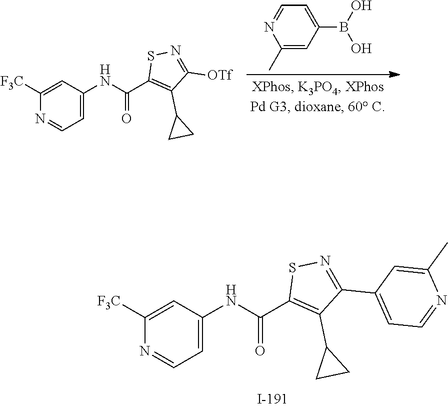

A solution of 4-cyclopropyl-5-{[2-(trifluoromethyl)pyridin-4-yl]carbamoyl}-1,2-thiazol-3-yl trifluoromethanesulfonate (100 mg, 0.217 mmol, 1 equiv) in 1,4-dioxane (2 mL) was treated with 2-methyl-4-(4,4,5,5-tetramethyl-1,3,2-dioxaborolan-2-yl)pyridine (71.2 mg, 0.326 mmol, 1.5 equiv) for 5 min at room temperature under a nitrogen atmosphere followed by the addition of XPhos (20.7 mg, 0.043 mmol, 0.2 equiv), K₃PO₄ (138 mg, 0.651 mmol, 3 equiv) and XPhos Pd G3 (73.39 mg, 0.087 mmol, 0.4 equiv) in portions at room temperature. The resulting mixture was stirred for an additional 2 h at 60° C. The residue was purified by reversed-phase flash chromatography with the following conditions: column: C18 silica gel; mobile phase: MeCN in water (0.1% TFA); 0% to 100% gradient in 20 min; detector: UV 254 nm. This resulted in 4-cyclopropyl-3-(2-methylpyridin-4-yl)-N-(2-(trifluoromethyl)pyridin-4-yl)isothiazole-5-carboxamide (I-191, 1.1 mg, 1.25%) as a white solid. LCMS (ES, m/z): [M+1]⁺=405; ¹H NMR (400 MHz, DMSO-d₆) δ 0.33 (q, J=5.2 Hz, 2H), 0.83-0.92 (m, 2H), 2.21-2.28 (m, 1H), 2.58 (s, 3H), 7.65 (d, J=5.2 Hz, 1H), 7.70 (s, 1H), 7.94 (dd, J=5.6, 1.9 Hz, 1H), 8.22 (d, J=2.0 Hz, 1H), 8.61 (d, J=5.2 Hz, 1H), 8.73 (d, J=5.6 Hz, 1H), 11.42 (s, 1H).

### EXAMPLE 276—SYNTHESIS OF 4-CYCLOPROPYL-3-(4-FLUOROPYRIDIN-3-YL)-N-(2-(TRIFLUOROMETHYL)PYRIDIN-4-YL)ISOTHIAZOLE-5-CARBOXAMIDE (I-192)

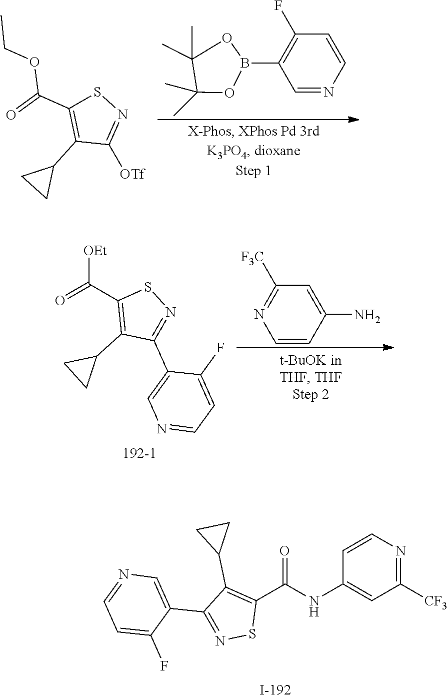 [SMILES: CCOC(=O)c1snc(OS(=O)(=O)C(F)(F)F)c1C1CC1 ; CC1(C)OB(c2cnccc2F)OC1(C)C ; CCOC(=O)c1snc(-c2cnccc2F)c1C1CC1 ; Nc1ccnc(C(F)(F)F)c1 ; O=C(Nc1ccnc(C(F)(F)F)c1)c1snc(-c2cnccc2F)c1C1CC1]

Step 1: Synthesis of 192-1: A solution of ethyl 4-cyclopropyl-3-(trifluoromethanesulfonyloxy)-1,2-thiazole-5-carboxylate (100 mg, 0.290 mmol, 1 equiv) in 1,4-dioxane (5 mL) was treated with 4-fluoro-3-(4,4,5,5-tetramethyl-1,3,2-dioxa-borolan-2-yl)pyridine (77.51 mg, 0.348 mmol, 1.2 equiv), XPhos (27.61 mg, 0.058 mmol, 0.2 equiv), XPhos Pd G₃ (36.77 mg, 0.043 mmol, 0.15 equiv) and K₃PO₄ (245.88 mg, 1.160 mmol, 4 equiv). The mixture was stirred overnight at 100° C. under a nitrogen atmosphere. The desired product could be detected by LCMS. The reaction was quenched by the addition of water (10 mL) at 0° C. The aqueous layer was extracted with EtOAc (3×50 mL). The organic phase was washed with the brine (50 mL) and dried over anhydrous Na₂SO₄. After filtration, the filtrate was concentrated under reduced pressure. The residue was purified by prep-TLC (PE/EA 10:1) to afford 192-1 (65 mg, 76.78%) as a yellow solid.

Step 2: Synthesis of 4-cyclopropyl-3-(4-fluoropyridin-3-yl)-N-(2-(trifluoromethyl)pyridin-4-yl)isothiazole-5-carboxamide (I-192): A solution of 192-1 (60 mg, 0.205 mmol, 1.00 equiv) in THF (2 mL) was treated with 2-(trifluoromethyl)pyridin-4-amine (49.91 mg, 0.307 mmol, 1.5 equiv) for 2 min at room temperature under a nitrogen atmosphere followed by the addition of t-BuOK (46.06 mg, 0.410 mmol, 2 equiv) at 0° C. The resulting mixture was stirred for an additional 30 min at room temperature. After the reaction was complete, the reaction was quenched with water (30 mL) at 0° C. The aqueous layer was extracted with EtOAc (3×80 mL). The organic phase was washed with the brine (100 mL) and dried over anhydrous Na₂SO₄. After filtration, the filtrate was concentrated under reduced pressure. The crude product (50 mg) was purified by prep-HPLC with the following conditions: (column: Xselect CSH Cis OBD column 30*150 mm 5 m, n; mobile phase A: water (0.1% FA), mobile phase B: ACN; flow rate: 60 mL/min; gradient: 44% B to 55% B in 8 min, 55% B; wavelength: 254; 220 nm; RT1(min): 7.47; injection volume: 1 mL; number of runs: 3) to afford 4-cyclopropyl-3-(4-fluoropyridin-3-yl)-N-(2-(trifluoromethyl)pyridin-4-yl)isothiazole-5-carboxamide (I-192, 7.8 mg, 9.29%) as a white solid. LCMS (ES, m/z): [M+H]⁺=409.07; ¹H NMR (400 MHz, DMSO-d₆) δ 0.28 (d, J=4.4 Hz. 2H), 0.65-0.76 (m, 2H), 2.04-2.15 (m, 1H), 7.57 (s, 1H), 7.95 (t, J=2.0, 5.6 Hz, 1H), 8.22 (d, J=1.6 Hz, 1H), 8.73 (d, J=5.2 Hz, 1H), 8.76-8.80 (m, 2H), 11.38 (s, 1H).

### EXAMPLE 277—SYNTHESIS OF 4-CYCLOPROPYL-3-(3-(METHYLCARBAMOYL) PHENYL)-N-(2-(TRIFLUOROMETHYL)PYRIDIN-4-YL)ISOTHIAZOLE-5-CARBOXAMIDE (I-193)

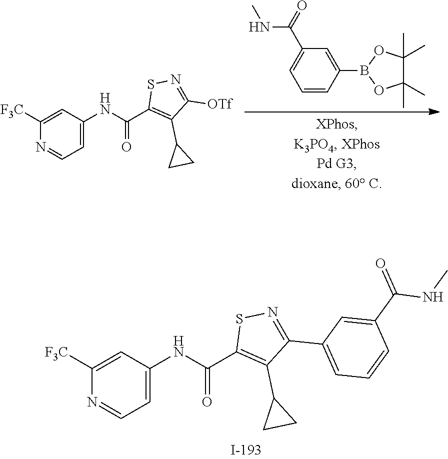

A solution of 4-cyclopropyl-5-{[2-(trifluoromethyl)pyridin-4-yl]carbamoyl}-1,2-thiazol-3-yl trifluoromethanesulfonate (100 mg, 0.217 mmol, 1 equiv) in 1,4-dioxane (2 mL) was treated with N-methyl-3-(4,4,5,5-tetramethyl-1,3,2-dioxaborolan-2-yl)benzamide (67.9 mg, 0.260 mmol, 1.2 equiv) for 5 min at room temperature under a nitrogen atmosphere followed by the addition of K₃PO₄ (138 mg, 0.651 mmol, 3 equiv) and XPhos Pd G3 (73.4 mg, 0.087 mmol, 0.4 equiv) in portions at room temperature. The resulting mixture was stirred for an additional 2 h at 60° C. The residue was purified by reversed-phase flash chromatography with the following conditions: column: C18 silica gel; mobile phase: MeCN in water (0.1% TFA); 0% to 100% gradient in 20 min; detector: UV 254 nm. This resulted in 4-cyclopropyl-3-(3-(methylcarbamoyl)phenyl)-N-(2-(trifluoromethyl)pyridin-4-yl)isothiazole-5-carboxamide (I-193, 14.4 mg, 14.85%) as a white solid. LCMS (ES, m/z): [M+1]⁺=447; ¹H NMR (400 MHz, DMSO-d₆) δ 0.29 (d, J=5.4 Hz, 2H), 0.77-0.88 (m, 2H), 2.22-2.29 (m, 1H), 2.81-2.83 (m, 3H), 7.63 (t, J=7.6 Hz, 1H), 7.94-8.00 (m, 3H), 8.23 (d, J=2.0 Hz, 1H), 8.29-8.34 (m, 1H), 8.59 (d, J=4.8 Hz, 1H), 8.74 (d, J=5.6 Hz, 1H), 11.39 (s, 1H).

### EXAMPLE 278—SYNTHESIS OF 4-CYCLOPROPYL-3-(IMIDAZO[1,5-A]PYRIDIN-6-YL)-N-(2-(TRIFLUOROMETHYL)PYRIDIN-4-YL)ISOTHIAZOLE-5-CARBOXAMIDE (I-194)

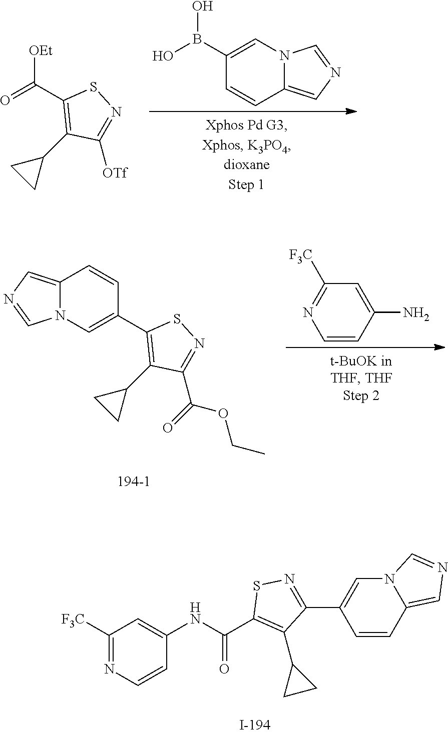 [SMILES: CCOC(=O)c1snc(OS(=O)(=O)C(F)(F)F)c1C1CC1 ; OB(O)c1ccc2cncn2c1 ; CCOC(=O)c1nsc(-c2ccc3cncn3c2)c1C1CC1 ; Nc1ccnc(C(F)(F)F)c1 ; O=C(Nc1ccnc(C(F)(F)F)c1)c1snc(-c2ccc3cncn3c2)c1C1CC1]

Step 1: Synthesis of 194-1: A solution of ethyl 4-cyclopropyl-3-(trifluoromethanesulfonyloxy)-1,2-thiazole-5-carboxylate (150 mg, 0.434 mmol, 1 equiv), imidazo[1,5-a]pyridin-6-ylboronic acid (84 mg, 0.519 mmol, 1.19 equiv), XPhos (42 mg, 0.088 mmol, 0.20 equiv), XPhos Pd G3 (74 mg, 0.087 mmol, 0.20 equiv) and K₃PO₄ (276 mg, 1.300 mmol, 2.99 equiv) in 1,4-dioxane (10 mL) was stirred overnight at 60° C. under a nitrogen atmosphere. The resulting mixture was diluted with water (10 mL). The resulting mixture was extracted with EtOAc (3×20 mL). The combined organic layers were washed with brine (1×40 mL) and dried over anhydrous Na₂SO₄. After filtration, the filtrate was concentrated under reduced pressure. The crude product (80 mg) was purified by prep-HPLC with the following conditions: (column: XBridge Prep OBD C18 column: 30*150 mm, 5 μm; mobile phase A: water (10 mmol/L NH₄HCO₃+0.1% NH₃·H₂O), mobile phase B: ACN; flow rate: 60 mL/min; gradient: 36% B to 65% B in 8 min, 65% B; wavelength: 220 nm; RT1(min): 7.47; number of runs: 1) to afford ethyl 4-cyclopropyl-3-{imidazo[1,5-a]pyridin-6-yl}-1,2-thiazole-5-carboxylate (194-1, 100 mg, 73.46%) as a light yellow oil.

Step 2: Synthesis of 4-cyclopropyl-3-(imidazo[1,5-a]pyridin-6-yl)-N-(2-(trifluoromethyl)pyridin-4-yl)isothiazole-5-carboxamide (I-194): A solution of ethyl 4-cyclopropyl-3-{imidazo[1,5-a]pyridin-6-yl}-1,2-thiazole-5-carboxylate (100 mg, 0.319 mmol, 1 equiv), 2-(trifluoromethyl)pyridin-4-amine (58 mg, 0.358 mmol, 1.12 equiv) and t-BuOK (108 mg, 0.962 mmol, 3.02 equiv) in THF (5 mL) was stirred for 0.5 h at room temperature under a nitrogen atmosphere. The resulting mixture was diluted with water (5 mL). The resulting mixture was extracted with EtOAc (3×10 mL). The combined organic layers were washed with brine (1×20 mL) and dried over anhydrous Na₂SO₄. After filtration, the filtrate was concentrated under reduced pressure. The crude product (50 mg) was purified by prep-HPLC with the following conditions: (column: Xselect CSH C18 OBD column 30*150 mm 5 μm, n; mobile phase A: water (0.1% FA), mobile phase B: ACN; flow rate: 60 mL/min; gradient: 24% B to 40% B in 8 min, 40% B; wavelength: 254; 220 nm; RT1(min): 7.3; injection volume: 1 mL; number of runs: 3) to afford 4-cyclopropyl-3-{imidazo[1,5-a]pyridin-6-yl}-N-[2-(trifluoromethyl) pyridin-4-yl]-1,2-thiazole-5-carboxamide (I-194, 20.7 mg, 14.91%) as an off-white solid. LCMS (ES, m/z): [M+1]⁺=430; ¹H NMR (400 MHz, DMSO-d₆) δ 0.30-0.55 (m, 2H), 0.82-1.10 (m, 2H), 2.13-2.40 (m, 1H), 7.32 (dd, J=9.6, 1.2 Hz, 1H), 7.44 (s, 1H), 7.67 (d, J=9.6 Hz, 1H), 7.94 (dd, J=5.6, 1.6 Hz, 1H), 8.23 (d, J=1.6 Hz, 1H), 8.56 (s, 1H), 8.74 (d, J=5.6 Hz, 1H), 9.00 (d, J=1.6 Hz, 1H), I 1.42 (s, 1H).

### EXAMPLE 279—SYNTHESIS OF 4-CYCLOPROPYL-3-(3-FLUOROPHENYL)-N-(2-(TRIFLUOROMETHYL)PYRIDIN-4-YL)ISOTHIAZOLE-5-CARBOXAMIDE (I-195)

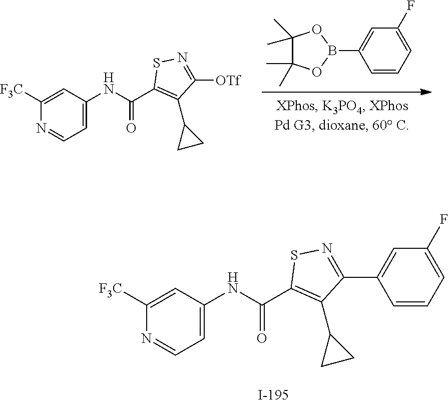 [SMILES: O=C(Nc1ccnc(C(F)(F)F)c1)c1snc(OS(=O)(=O)C(F)(F)F)c1C1CC1 ; CC1(C)OB(c2cccc(F)c2)OC1(C)C ; O=C(Nc1ccnc(C(F)(F)F)c1)c1snc(-c2cccc(F)c2)c1C1CC1]

To a stirred solution of 4-cyclopropyl-5-{[2-(trifluoromethyl) pyridin-4-yl]carbamoyl}-1,2-thiazol-3-yl trifluoromethanesulfonate (90 mg, 0.195 mmol, 1 equiv) and 2-(3-fluorophenyl)-4,4,5,5-tetramethyl-1,3,2-dioxaborolane (51.99 mg, 0.234 mmol, 1.2 equiv) in 1,4-dioxane (2 mL) was added XPhos (9.30 mg, 0.020 mmol, 0.1 equiv), XPhos Pd G3 (16.51 mg, 0.020 mmol, 0.1 equiv) and K₃PO₄ (103.52 mg, 0.488 mmol, 2.5 equiv) in portions at room temperature under a nitrogen atmosphere. The resulting mixture was stirred for an additional 2 h at 60° C. The resulting mixture was diluted with water (10 mL). The resulting mixture was extracted with EtOAc (3×20 mL). The combined organic layers were washed with brine (1×10 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 50% to 60% gradient in 10 min; detector: UV 254 nm. The resulting mixture was concentrated under reduced pressure. This resulted in 4-cyclopropyl-3-(3-fluorophenyl)-N-[2-(trifluoromethyl) pyridin-4-yl]-1,2-thiazole-5-carboxamide (I-195, 1.5 mg, 1.88%) as a white solid. LCMS (ES, m/z): [M+H]⁺=408; ¹H NMR (400 MHz, DMSO-d₆) δ 0.33 (d, J=5.6 Hz, 2H), 0.86 (d, J=8.0 Hz, 2H), 2.19-2.31 (m, 1H), 7.37 (t, J=8.0 Hz, 1H), 7.62 (dd, J=18.4, 9.0 Hz, 2H), 7.72 (d, J=8.0 Hz, 1H), 7.94 (d, J=5.6 Hz, 1H), 8.22 (s, 1H), 8.73 (d, J=5.6 Hz, 1H), 11.40 (s, 1H).

### EXAMPLE 280—SYNTHESIS OF 4-CYCLOPROPYL-N-(2-METHYLPYRIDIN-4-YL)-3-PHENYLISOTHIAZOLE-5-CARBOXAMIDE (1-196)

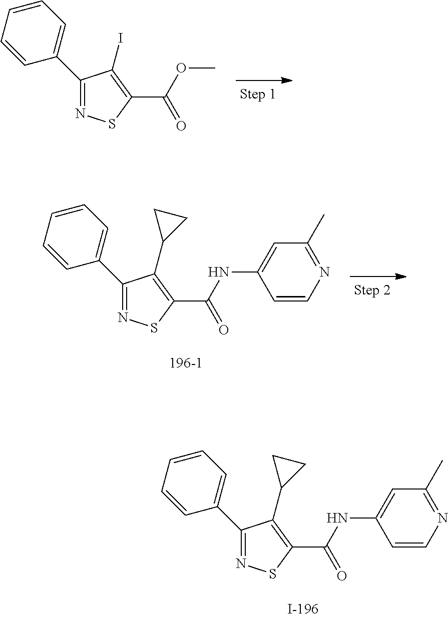 [SMILES: COC(=O)c1snc(-c2ccccc2)c1I ; Cc1cc(NC(=O)c2snc(-c3ccccc3)c2C2CC2)ccn1 ; Cc1cc(NC(=O)c2snc(-c3ccccc3)c2C2CC2)ccn1]

Step 1: Synthesis of 196-1: A solution of methyl 4-iodo-3-phenyl-1,2-thiazole-5-carboxylate (2 g, 5.795 mmol, 1 equiv), Pd(PPh₃)₂Cl₂ (0.81 g, 1.159 mmol, 0.2 equiv), cyclopropylboronic acid (0.75 g, 8.692 mmol, 1.5 equiv) and Ag₂O (2.69 g, 11.590 mmol, 2 equiv) in dioxane (30 mL) was stirred for 2 h at 90° C. under a nitrogen atmosphere. The resulting mixture was diluted with water (50 mL). The resulting mixture was extracted with EtOAc (3×50 mL). The combined organic layers were washed with brine (1×150 mL) and dried over anhydrous Na₂SO₄. After filtration, the filtrate was concentrated under reduced pressure. The residue was purified by silica gel column chromatography and eluted with PE/EA (10:1) to afford methyl 4-cyclopropyl-3-phenyl-1,2-thiazole-5-carboxylate (196-1, 1.2 g, 75.07%) as a light yellow oil. LCMS (ES, m/z): [M+H]⁺⁼260.

Step 2: Synthesis of 4-cyclopropyl-N-(2-methylpyridin-4-yl)-3-phenylisothiazole-5-carboxamide (I-196): To a stirred solution of ethyl 4-cyclopropyl-3-{imidazo[1,2-a]pyridin-7-yl}-1,2-thiazole-5-carboxylate (100 mg, 0.319 mmol, 1 equiv), 2-(trifluoromethyl)pyridin-4-amine (57 mg, 0.351 mmol, 1.1 equiv) in THF (2 mL) was added t-BuOK (130 mg, 1.158 mmol, 3 equiv) at 0° C. under a nitrogen atmosphere. The resulting mixture was stirred for 2 h at room temperature under a nitrogen atmosphere. The resulting mixture was diluted with water (10 mL). The resulting mixture was extracted with EtOAc (3×10 mL). The combined organic layers were washed with brine (1×30 mL) and dried over anhydrous Na₂SO₄. After filtration, the filtrate was concentrated under reduced pressure. The crude product was purified by prep-HPLC with the following conditions: column: XBridge Prep OBD C18 column: 30*150 mm, 5 μm; mobile phase A: water (10 mmol/L NH₄HCO₃), mobile phase B: ACN; flow rate: 60 mL/min; gradient: 35% B to 58% B in 8 min, 58% B; wavelength: 254 nm; RT1(min): 7.67 to afford 4-cyclopropyl-3-{imidazo[1,2-a]pyridin-7-yl}-N-[2-(trifluoromethyl)pyridin-4-yl]-1,2-thiazole-5-carboxamide (I-196, 67.3 mg, 49.06%) as an off-white solid. LCMS (ES, m/z): [M+H]⁺=336; ¹H NMR (400 MHz, DMSO-d₆) δ 0.28-0.35 (m, 2H), 0.76-0.87 (m, 2H), 2.10-2.30 (m, 1H), 2.46 (s, 3H), 7.46-7.61 (m, 5H), 7.83 (d, J=1.2 Hz, 1H), 7.84 ((d, J=1.6 Hz, 1H), 8.39 (d, J=5.6 Hz, 1H), 10.93 (s, 1H).

### EXAMPLE 281—SYNTHESIS OF 4-CYCLOPROPYL-3-(3,5-DIMETHYLISOXAZOL-4-YL)-N-(2-(TRIFLUOROMETHYL)PYRIDIN-4-YL)ISOTHIAZOLE-5-CARBOXAMIDE (I-197)

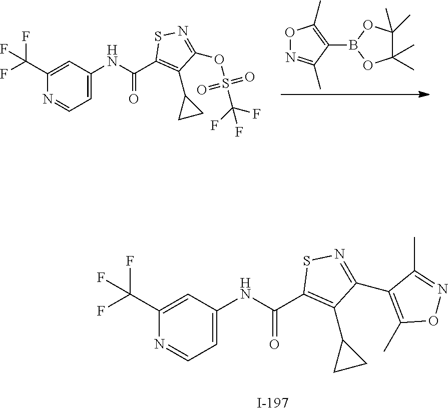 [SMILES: O=C(Nc1ccnc(C(F)(F)F)c1)c1snc(OS(=O)(=O)C(F)(F)F)c1C1CC1 ; Cc1noc(C)c1B1OC(C)(C)C(C)(C)O1 ; Cc1noc(C)c1-c1nsc(C(=O)Nc2ccnc(C(F)(F)F)c2)c1C1CC1]

A solution of 4-cyclopropyl-5-{[2-(trifluoromethyl)pyridin-4-yl]carbamoyl}-1,2-thiazol-3-yl trifluoromethanesulfonate (100 mg, 0.217 mmol, 1 equiv) in 1,4-dioxane (2 mL) was treated with 3,5-dimethyl-4-(4,4,5,5-tetramethyl-1,3,2-dioxaborolan-2-yl)-1,2-oxazole (58.02 mg, 0.260 mmol, 1.2 equiv) for 5 min at room temperature under a nitrogen atmosphere followed by the addition of K₃PO₄ (138.03 mg, 0.651 mmol, 3 equiv) and XPhos Pd G3 (36.74 mg, 0.043 mmol, 0.2 equiv) in portions at 60° C. The resulting mixture was stirred for an additional 2 h at 60° C. The residue was purified by reversed-phase flash chromatography with the following conditions: column: C18 silica gel; mobile phase: MeCN in water (0.1% TFA); 0% to 100% gradient in 20 min; detector: UV 254 nm. This resulted in 4-cyclopropyl-3-(3,5-dimethylisoxazol-4-yl)-N-(2-(trifluoromethyl)pyridin-4-yl)isothiazole-5-carboxamide (1-197, 2.3 mg, 2.55%) as a white solid. LCMS (ES, m/z): [M+H]⁺=409; ¹H NMR (400 MHz, DMSO-d) 60.36 (d, J=5.2 Hz, 2H), 0.79 (d, J=9.6 Hz, 2H), 2.01-2.06 (m, 1H), 2.21 (s, 3H), 2.42 (s, 3H), 7.90 (d, J=5.6 Hz, 1H), 8.19 (s, 1H), 8.67 (d, J=5.6 Hz, 1H).

### EXAMPLE 282—SYNTHESIS OF N-(3-CHLORO-4-(3-HYDROXYAZETIDINE-1-CARBONYL)PHENYL)-4-CYCLOPROPYL-3-(1-OXO-1,2-DIHYDROISOQUINOLIN-5-YL)ISOTHIAZOLE-5-CARBOXAMIDE (1-198)

Step 1: Synthesis of 198-1: To a stirred mixture of ethyl 4-cyclopropyl-3-(1-methoxyisoquinolin-5-yl)-1,2-thiazole-5-carboxylate (570 mg, 1.608 mmol, 1 equiv) in HOAc (5 mL) was added HBr (390.38 mg, 4.824 mmol, 3.00 equiv) in portions at room temperature under a nitrogen atmosphere. The resulting mixture was stirred for an additional 3 h at room temperature. The reaction was quenched with MeOH at room temperature. The residue was purified by reversed-phase flash chromatography with the following conditions: column: C18 silica gel; mobile phase: MeCN in water (0.1% FA), 10% to 50% gradient in 10 min; detector: UV 254 nm. This resulted in ethyl 4-cyclopropyl-3-(1-oxo-2H-isoquinolin-5-yl)-1,2-thiazole-5-carboxylate (198-1, 180 mg, 29.59%) as a white solid.

Step 2: Synthesis of 198-2: To a stirred mixture of ethyl 4-cyclopropyl-3-(1-oxo-2H-isoquinolin-5-yl)-1,2-thiazole-5-carboxylate (130 mg, 0.382 mmol, 1 equiv) and 4-{3-[(tert-butyldimethylsilyl)oxy]azetidine-1-carbonyl}-3-chloroaniline (130.20 mg, 0.382 mmol, 1 equiv) in THF (2 mL) was added t-BuOK (128.56 mg, 1.146 mmol, 3 equiv) in portions at room temperature under a nitrogen atmosphere. The resulting mixture was stirred for an additional 0.5 h at room temperature. The resulting mixture was diluted with water (30 mL). The resulting mixture was extracted with EtOAc (3×50 mL). The combined organic layers were washed with brine (1×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 10% to 50% gradient in 10 min; detector: UV 254 nm. This resulted in N-(4-{3-[(tert-butyldimethylsilyl)oxy]azetidine-1-carbonyl}-3-chlorophenyl)-4-cyclopropyl-3-(1-oxo-2H-isoquinolin-5-yl)-1,2-thiazole-5-carboxamide (198-2, 40 mg, 16.49%) as a white solid.

Step 3: Synthesis of N-(3-chloro-4-(3-hydroxyazetidine-1-carbonyl)phenyl)-4-cyclopropyl-3-(1-oxo-1,2-dihydroisoquinolin-5-yl)isothiazole-5-carboxamide (I-198): To a stirred mixture of N-(4-{3-[(tert-butyldimethylsilyl)oxy]azetidine-1-carbonyl}-3-chlorophenyl)-4-cyclopropyl-3-(1-oxo-2H-isoquinolin-5-yl)-1,2-thiazole-5-carboxamide (40 mg, 0.063 mmol, 1 equiv) in DMF (2 mL) was added CsF (19.13 mg, 0.126 mmol, 2 equiv) in portions at room temperature under a nitrogen atmosphere. The resulting mixture was stirred for an additional 3 h at room temperature. The resulting mixture was diluted with water (30 mL). The resulting mixture was extracted with EtOAc (3×50 mL). The combined organic layers were washed with brine (1×20 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% FA), 10% to 50% gradient in 10 min; detector: UV 254 nm. This resulted in N-[3-chloro-4-(3-hydroxyazetidine-1-carbonyl)phenyl]-4-cyclopropyl-3-(1-oxo-2H-isoquinolin-5-yl)-1,2-thiazole-5-carboxamide (1-198, 25.8 mg, 77.39%) as a white solid. LCMS (ES, m/z): [M+H]⁺=521; ¹H NMR (400 MHz, DMSO-d₆): δ 0.26-0.32 (m, 2H), 0.54-0.63 (m, 2H), 1.90 (m, 1H), 3.74 (m, J=21.0, 9.8, 4.4 Hz, 2H), 4.06 (t, J=7.8 Hz, 1H), 4.19-4.27 (m, 1H), 4.47-4.56 (m, 1H), 5.82 (d, J=6.2 Hz, 1H), 6.23 (d, J=7.4 Hz, 1H), 7.20 (dd, J=7.4, 5.8 Hz, 1H), 7.46 (d, J=8.4 Hz, 1H), 7.62 (t, J=7.8 Hz, 1H), 7.68 (dd, J=8.0, 2.0 Hz, 1H), 7.77 (dd, J=7.4, 1.4 Hz, 1H), 8.05 (s, 1H), 7.95 (d, J=2.0 Hz, 1H), 8.35 (d, J=8.0 Hz, 1H), 10.94 (s, 1H), 11.43 (d, J=5.8 Hz, 1H).

### EXAMPLE 283—SYNTHESIS OF 4-CYCLOPROPYL-N-(2-METHYLPYRIDIN-4-YL)-3-(QUINOLIN-5-YL)ISOTHIAZOLE-5-CARBOXAMIDE (1-199)

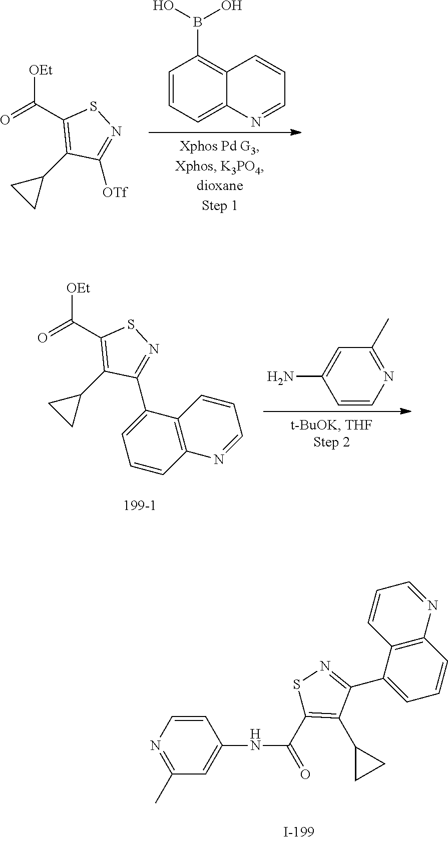

Step 1: Synthesis of 199-1: A mixture of ethyl 4-cyclopropyl-3-(trifluoromethanesulfonyloxy)-1,2-thiazole-5-carboxylate (210 mg, 0.608 mmol, 1 equiv), XPhos Pd G₃ (51.48 mg, 0.061 mmol, 0.1 equiv), XPhos (57.98 mg, 0.122 mmol, 0.2 equiv), K₃PO₄ (387.27 mg, 1.824 mmol, 3 equiv), and quinolin-5-ylboronic acid (126.24 mg, 0.730 mmol, 1.2 equiv) in 1,4-dioxane(5 mL) was stirred for 14 h at 60° C. under a nitrogen atmosphere. After the reaction was complete, the residue was dissolved in water (10 mL). The residue was washed with EtOAc (3×40 mL). The resulting mixture was concentrated under reduced pressure. The residue was purified by prep-TLC (PE/EA 8:1) to afford 199-1 (190 mg, 96.31%) as a brown solid.

Step 2: Synthesis of 4-cyclopropyl-N-(2-methylpyridin-4-yl)-3-(quinolin-5-yl)isothiazole-5-carboxamide (I-199): A solution of 199-1 (96 mg, 0.296 mmol, 1 equiv) in THF (5 mL) was treated with 2-methylpyridin-4-amine (38.40 mg, 0.355 mmol, 1.2 equiv) for 5 mins at 0° C. under a nitrogen atmosphere followed by the addition of t-BuOK (66.41 mg, 0.592 mmol, 2 equiv) at 0° C. The resulting mixture was stirred for 0.5 h at room temperature under a nitrogen atmosphere. The reaction was quenched by the addition of water (100 mL) at 0° C. The aqueous layer was extracted with EtOAc (3×80 mL). The resulting mixture was concentrated under reduced pressure. The crude product (100 mg) was purified by prep-HPLC with the following conditions: (column: XBridge Prep OBD Cis column: 30*150 mm, 5 μm; mobile phase A: water (10 mmol/L NH₄HCO₃+0.1% NH₃·H₂O), mobile phase B: ACN; flow rate: 60 mL/min; gradient: 22% B to 52% B in 8 min, 52% B; wavelength: 220 nm; RT1(min): 7.57) to afford 4-cyclopropyl-N-(2-methylpyridin-4-yl)-3-(quinolin-5-yl)isothiazole-5-carboxamide (I-199, 39.4 mg, 34.38%) as a white solid. LCMS (ES, m/z): [M+H]⁺=387.05; ¹H NMR (400 MHz, DMSO-d₆) δ 0.23 (d, J=4.4 Hz, 2H), 0.50-0.53 (m, 2H), 1.92 (s, 1H), 2.50 (s, 3H), 7.51-7.53 (m, 1H), 7.57-7.61 (m, 2H), 7.75 (d, J=6.8 Hz, 1H), 7.92 (d, J=8.0 Hz, 1H), 8.16 (d, J=8.0 Hz, 1H), 8.18 (s, 1H), 8.40 (d, J=5.6 Hz, 1H), 8.99 (dd, J=1.6, 2.0 Hz, 1H), 10.94 (s, 1H).

### EXAMPLE 284—SYNTHESIS OF 4-CYCLOPROPYL-N-(2-METHYLPYRIDIN-4-YL)-3-(1-OXO-1,2-DIHYDROISOQUINOLIN-5-YL)ISOTHIAZOLE-5-CARBOXAMIDE (I-200)

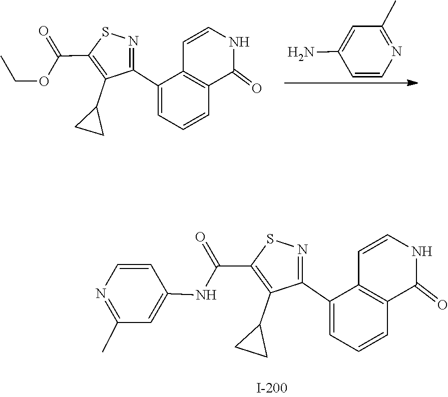

A solution of ethyl 4-cyclopropyl-3-(1-oxo-2H-isoquinolin-5-yl)-1,2-thiazole-5-carboxylate (100 mg, 0.294 mmol, 1 equiv) in DMF (2 mL) was treated with 2-methylpyridin-4-amine (47.65 mg, 0.441 mmol, 1.5 equiv) for 2 min at room temperature under a nitrogen atmosphere followed by the addition of t-BuOK (65.93 mg, 0.588 mmol, 2 equiv) dropwise at 60° C. The resulting mixture was stirred for an additional 2 h at 60° C. The residue was purified by reversed-phase flash chromatography with the following conditions: column: C18 silica gel; mobile phase: MeCN in water (0.1% TFA); 0% to 100% gradient in 20 min; detector: UV 254 nm. This resulted in 4-cyclopropyl-N-(2-methylpyridin-4-yl)-3-(1-oxo-1,2-dihydroisoquinolin-5-yl)isothiazole-5-carboxamide (1-200, 1.8 mg, 1.36%) as a white solid. LCMS (ES, m/z): [M+1]⁺=403; ¹H NMR (400 MHz, DMSO-d₆) δ 0.26 (q, J=5.2 Hz, 2H), 0.52-0.62 (m, 2H), 1.88-1.92 (m, 1H), 2.47 (s, 3H), 6.25 (d, J=7.2 Hz, 1H), 7.20 (t, J=6.4 Hz, 1H), 7.51 (d, J=5.6 Hz, 1H), 7.58 (s, 1H), 7.61 (t, J=7.7 Hz, 1H), 7.78 (d, J=7.2 Hz, 1H), 8.35 (d, J=8.0 Hz, 1H), 8.39 (d, J=5.6 Hz, 1H), 10.95 (s, 1H), 11.44 (d, J=5.6 Hz, 1H).

### EXAMPLE 285—SYNTHESIS OF (S)-4-CYCLOPROPYL-N-(2-METHYLPYRIDIN-4-YL)-3-(TETRAHYDROFURAN-3-YL)ISOTHIAZOLE-5-CARBOXAMIDE (I-201)

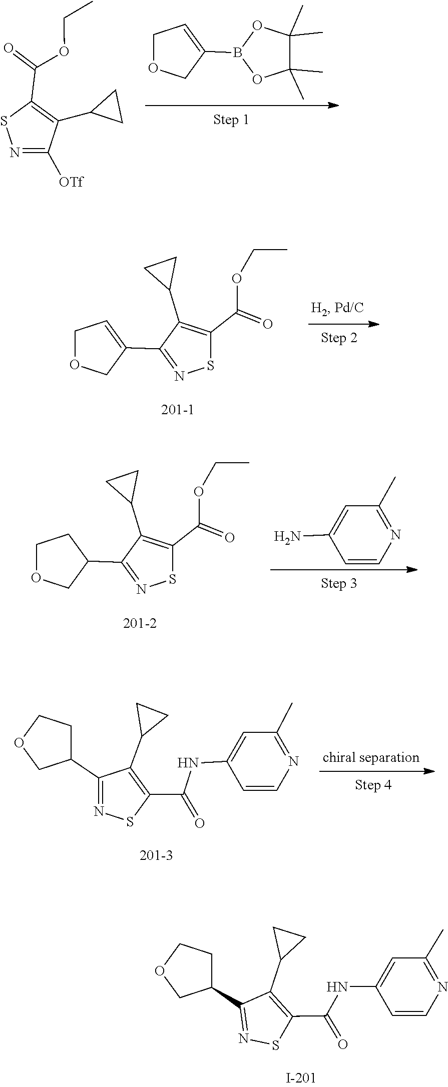

Step 1: Synthesis of 201-1: To a stirred solution of ethyl 4-cyclopropyl-3-(trifluoromethanesulfonyloxy)-1,2-thiazole-5-carboxylate (1 g, 2.896 mmol, 1 equiv) and 2-(2,5-dihydrofuran-3-yl)-4,4,5,5-tetramethyl-1,3,2-dioxaborolane (625 mg, 3.186 mmol, 1.1 equiv) in dioxane (10 mL) was added K₃PO₄ (1844 mg, 8.688 mmol, 3 equiv), XPhos (276 mg, 0.579 mmol, 0.2 equiv) and XPhos Pd G3 (245 mg, 0.290 mmol, 0.1 equiv). The resulting mixture was stirred for 2 h at 60° C. under a nitrogen atmosphere. The resulting mixture was diluted with water (30 mL). The resulting mixture was extracted with EtOAc (3×30 mL). The combined organic layers were washed with brine (1×100 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% TFA); 10% to 50% gradient in 10 min; detector: UV 254 nm to afford ethyl 4-cyclopropyl-3-(2,5-dihydrofuran-3-yl)-1,2-thiazole-5-carboxylate (201-1, 700 mg, 91.10%) as a light yellow oil. LCMS (ES, m/z): [M+1]⁺=266.

Step 2: Synthesis of 201-2: A mixture of ethyl 4-cyclopropyl-3-(2,5-dihydrofuran-3-yl)-1,2-thiazole-5-carboxylate (700 mg, 2.638 mmol, 1 equiv) and Pd/C (56 mg, 0.528 mmol, 0.2 equiv) in EtOH (10 mL) was stirred overnight at room temperature under a hydrogen atmosphere. The resulting mixture was filtered, and the filter cake was washed with EtOH (5×10 mL). The filtrate was concentrated under reduced pressure. The residue was purified by reversed-phase flash chromatography with the following conditions: column: C18 silica gel; mobile phase: MeCN in water (0.1% TFA); 10% to 50% gradient in 10 min; detector: UV 254 nm to afford ethyl 4-cyclopropyl-3-(oxolan-3-yl)-1,2-thiazole-5-carboxylate (201-2, 400 mg, 56.71%) as a light yellow oil. LCMS (ES, m/z): [M+1]⁺=268.

Step 3: Synthesis of 201-3: To a stirred solution of ethyl 4-cyclopropyl-3-(oxolan-3-yl)-1,2-thiazole-5-carboxylate (100 mg, 0.37 mmol, 1 equiv) and 2-methylpyridin-4-amine (53 mg, 0.48 mmol, 1.3 equiv) in THF (1 mL) was added t-BuOK (85 mg, 0.74 mmol, 2 equiv) at 0° C. The resulting mixture was stirred for 1 h at room temperature. The resulting mixture was diluted with water (20 mL). The resulting mixture was extracted with EtOAc (3×20 mL). The combined organic layers were washed with brine (lx 50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% TFA); 10% to 50% gradient in 10 min; detector: UV 254 nm to afford 4-cyclopropyl-N-(2-methylpyridin-4-yl)-3-(tetrahydrofuran-3-yl)isothiazole-5-carboxamide (201-3, 150 mg, 59.89%) as a light yellow oil. LCMS (ES, m/z): [M+1]⁺=330.

Step 4: Synthesis of (S)-4-cyclopropyl-N-(2-methylpyridin-4-yl)-3-(tetrahydrofuran-3-yl)isothiazole-5-carboxamide (1-201): 4-cyclopropyl-N-(2-methylpyridin-4-yl)-3-(tetrahydrofuran-3-yl)isothiazole-5-carboxamide (150 mg) was purified by prep-HPLC with the following conditions: (column: CHIRALPAK IF-3, 4.6*50 mm, 3 μm; mobile phase A: hex (0.2% DEA):EtOH=90:10; flow rate: 1 mL/min; gradient: 0% B to 0% B) to afford 4-cyclopropyl-N-(2-methylpyridin-4-yl)-3-[(3S)-oxolan-3-yl]-1,2-thiazole-5-carboxamide (I-201, 42.9 mg, 17.13%) as a white solid. LCMS (ES, m/z): [M+1]⁺=330; ¹H NMR (400 MHz, DMSO-d₆) δ 0.44-0.57 (m, 2H), 0.87-0.96 (m, 2H), 1.92-1.98 (m, 1H), 2.19-2.25 (m, 1H), 2.37-2.27 (m, 1H), 2.65 (s, 3H), 3.98-3.76 (m, 4H), 4.14 (t, J=7.6 Hz, 1H), 7.87 (dd, J=1.6, 6.8 Hz, 1H), 7.97 (d, J=2.0 Hz, 1H), 8.63 (d, J=6.8 Hz, 1H), 11.64 (s, 1H).

### EXAMPLE 286—SYNTHESIS OF (R)-4-CYCLOPROPYL-N-(2-METHYLPYRIDIN-4-YL)-3-(TETRAHYDROFURAN-3-YL)ISOTHIAZOLE-5-CARBOXAMIDE (1-202)

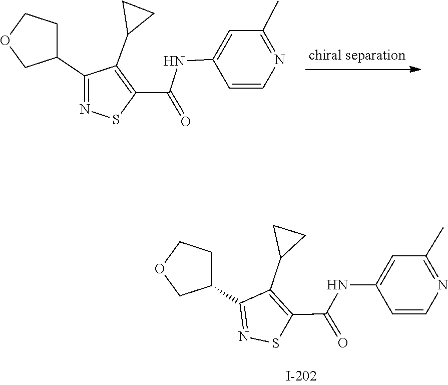 [SMILES: Cc1cc(NC(=O)c2snc(C3CCOC3)c2C2CC2)ccn1 ; Cc1cc(NC(=O)c2snc([C@H]3CCOC3)c2C2CC2)ccn1]

4-Cyclopropyl-N-(2-methylpyridin-4-yl)-3-(tetrahydrofuran-3-yl)isothiazole-5-carboxamide (150 mg) was purified by prep-HPLC with the following conditions: (column: CHIRALPAK IF-3, 4.6*50 mm, 3 μm; mobile phase A: hex (0.2% DEA):EtOH=90:10; flow rate: 1 mL/min; gradient: 0% B to 0% B) to afford 4-cyclopropyl-N-(2-methylpyridin-4-yl)-3-[(3R)-oxolan-3-yl]-1,2-thiazole-5-carboxamide (1-202, 26.0 mg, 10.51%) as a white solid. LCMS (ES, m/z): [M+1]⁺=330; ¹H NMR (400 MHz, DMSO-d₆) δ 0.48-0.57 (m, 2H), 0.89-0.99 (m, 2H), 1.93-1.99 (m, 1H), 2.18-2.26 (m, 1H), 2.30-2.38 (m, 1H), 2.65 (s, 3H), 3.94-3.78 (m, 4H), 4.14 (t, J=7.6 Hz, 1H), 7.86 (dd, J=2.0, 6.8 Hz, 1H), 7.96 (d, J=2.0 Hz, 1H), 8.62 (d, J=6.4 Hz, 1H), 11.60 (s, 1H).

### EXAMPLE 287—SYNTHESIS OF N-(3-CHLORO-4-(3-HYDROXYAZETIDINE-1-CARBONYL)PHENYL)-4-CYCLOPROPYL-3-(2-METHYL-2H-INDAZOL-4-YL)ISOTHIAZOLE-5-CARBOXAMIDE (I-203)

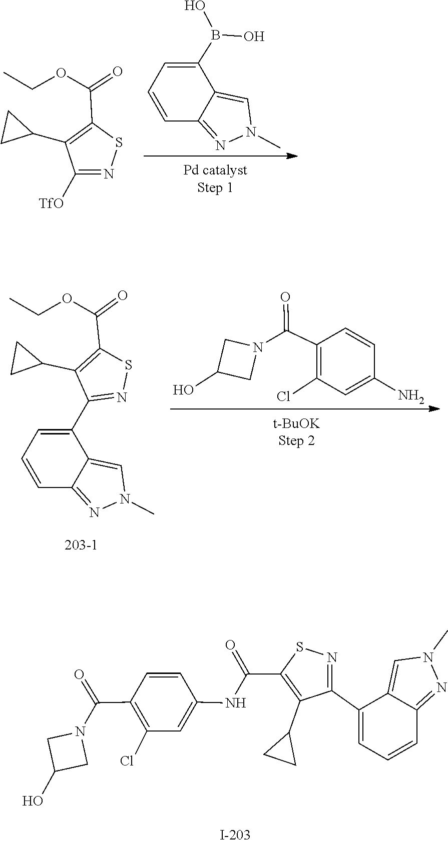

Step 1: Synthesis of 203-1: A solution of ethyl 4-cyclopropyl-3-(trifluoromethanesulfonyloxy)-1,2-thiazole-5-carboxylate (800 mg, 2.317 mmol, 1 equiv), 2-methyl-4-(4,4,5,5-tetramethyl-1,3,2-dioxaborolan-2-yl)indazole (897 mg, 3.476 mmol, 1.5 equiv), XPhos Pd G3 (392 mg, 0.463 mmol, 0.2 equiv), XPhos (276 mg, 0.579 mmol, 0.25 equiv) and K₃PO₄ (1475 mg, 6.951 mmol, 3 equiv) in dioxane (10 mL) was stirred for 2 h at 60° C. under a nitrogen atmosphere. The resulting mixture was diluted with water (50 mL). The resulting mixture was extracted with EtOAc (3×50 mL). The combined organic layers were washed with brine (1×100 mL) and dried over anhydrous Na₂SO₄. After filtration, the filtrate was concentrated under reduced pressure. The residue was purified by silica gel column chromatography and eluted with PE/EA (3:1) to afford ethyl 4-cyclopropyl-3-(2-methylindazol-4-yl)-1,2-thiazole-5-carboxylate (203-1, 700 mg, crude) as a black solid. LCMS (ES, m/z): [M+H]⁺=328.

Step 2: Synthesis of N-(3-chloro-4-(3-hydroxyazetidine-1-carbonyl)phenyl)-4-cyclopropyl-3-(2-methyl-2H-indazol-4-yl)isothiazole-5-carboxamide (I-203): To a stirred solution of ethyl 4-cyclopropyl-3-(2-methylindazol-4-yl)-1,2-thiazole-5-carboxylate (100 mg, 0.305 mmol, 1 equiv) and 4-{3-[(tert-butyldimethylsilyl)oxy]azetidine-1-carbonyl}-3-chloroaniline (115 mg, 0.336 mmol, 1.1 equiv) in THF (2 mL) was added t-BuOK (103 mg, 0.915 mmol, 3 equiv) at 0° C. under a nitrogen atmosphere. The resulting mixture was stirred for 1 h at room temperature under a nitrogen atmosphere. The resulting mixture was diluted with water (10 mL). The resulting mixture was extracted with EtOAc (3×10 mL). The combined organic layers were washed with brine (1×30 mL) and dried over anhydrous Na₂SO₄. After filtration, the filtrate was concentrated under reduced pressure. The crude product was purified by prep-HPLC with the following conditions: column: XBridge Prep OBD C18 column: 30*150 mm, 5 μm; mobile phase A: water (10 mmol/L NH₄HCO₃), mobile phase B: ACN; flow rate: 60 mL/min; gradient: 24% B to 38% B in 8 min; wavelength: 254 nm; RT1(min): 7.88 to afford N-[3-chloro-4-(3-hydroxyazetidine-1-carbonyl)phenyl]-4-cyclopropyl-3-(2-methylindazol-4-yl)-1,2-thiazole-5-carboxamide (I-203, 48.4 mg, 31.16%) as an off-white solid. LCMS (ES, m/z): [M+H]⁺=508; ¹H NMR (400 MHz, DMSO-d₆) δ 0.25-0.45 (m, 2H), 0.77-0.94 (m, 2H), 2.18-2.33 (m, 1H), 3.60-3.85 (m, 2H), 4.07-4.11 (t, J=8.0 Hz, 1H), 4.15-4.28 (m, 4H), 4.45-4.65 (m, 1H), 5.84 (d, J=6.0 Hz, 1H), 7.27 (dd, J=1.2, 8.0 Hz, 1H), 7.41 (d, J=8.0 Hz, 1H), 7.62-7.80 (m, 3H), 7.97 (d, J=1.6 Hz, 1H), 8.55 (s, 1H), 10.97 (s, 1H).

### EXAMPLE 288—SYNTHESIS OF (S)-4-CYCLOPROPYL-3-(TETRAHYDROFURAN-3-YL)-N-(2-(TRIFLUOROMETHYL)PYRIDIN-4-YL)ISOTHIAZOLE-5-CARBOXAMIDE (I-204)

Step 1: Synthesis of 204-1: To a stirred solution of ethyl 4-cyclopropyl-3-(trifluoromethanesulfonyloxy)-1,2-thiazole-5-carboxylate (1 g, 2.896 mmol, 1 equiv) and 2-(2,5-dihydrofuran-3-yl)-4,4,5,5-tetramethyl-1,3,2-dioxaborolane (624.53 mg, 3.186 mmol, 1.1 equiv) in dioxane (12 mL) was added K₃PO₄ (1844.13 mg, 8.688 mmol, 3 equiv), XPhos (276.12 mg, 0.579 mmol, 0.2 equiv) and XPhos Pd G3 (245.13 mg, 0.290 mmol, 0.1 equiv) in portions. The resulting mixture was stirred for 2 h at 60° C. under a nitrogen atmosphere. The reaction was quenched with water at room temperature. The resulting mixture was extracted with EtOAc (3×100 mL). The combined organic layers were washed with brine (100 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% TFA); 10% to 50% gradient in 10 min; detector: UV 254 nm. This resulted in ethyl 4-cyclopropyl-3-(2,5-dihydrofuran-3-yl)-1,2-thiazole-5-carboxylate (204-1, 520 mg, 67.20%) as a light yellow oil.

Step 2: Synthesis of 204-2: A mixture of ethyl 4-cyclopropyl-3-(2,5-dihydrofuran-3-yl)-1,2-thiazole-5-carboxylate (500 mg, 1.884 mmol, 1 equiv) and Pd/C (100 mg, 0.940 mmol, 0.50 equiv) in EtOH (6 mL) was stirred for 48 h at 70° C. under a hydrogen atmosphere. The resulting mixture was filtered, and the filter cake was washed with EtOH (5×10 mL). The filtrate was concentrated under reduced pressure. The residue was purified by reversed-phase flash chromatography with the following conditions: column: C18 silica gel; mobile phase: MeCN in water (0.1% TFA); 10% to 50% gradient in 10 min; detector: UV 254 nm to afford ethyl 4-cyclopropyl-3-(oxolan-3-yl)-1,2-thiazole-5-carboxylate (204-2, 190 mg, 30.21%) as a light yellow oil.

Step 3: Synthesis of 204-3: A solution of ethyl 4-cyclopropyl-3-(oxolan-3-yl)-1,2-thiazole-5-carboxylate (177 mg, 0.662 mmol, 1 equiv) in THF (4 mL) was treated with 2-(trifluoromethyl)pyridin-4-amine (139.53 mg, 0.861 mmol, 1.3 equiv) followed by the addition of t-BuOK (148.59 mg, 1.324 mmol, 2 equiv) at 0° C. The resulting mixture was stirred for 1 h at room temperature. The reaction was quenched with water at room temperature. The resulting mixture was extracted with EtOAc (3×50 mL). The combined organic layers were washed with brine (50 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water (0.1% TFA); 10% to 50% gradient in 10 min; detector: UV 254 nm. This resulted in 4-cyclopropyl-3-(oxolan-3-yl)-N-[2-(trifluoromethyl)pyridin-4-yl]-1,2-thiazole-5-carboxamide (204-3, 170 mg, 57.80%) as a light orange oil.

Step 4: (S)-4-cyclopropyl-3-(tetrahydrofuran-3-yl)-N-(2-(trifluoromethyl)pyridin-4-yl)isothiazole-5-carboxamide (I-204): 204-3 (170 mg) was purified by prep-chiral-HPLC with the following conditions: (column: CHIRALPAK IF, 2*25 cm, 5 m; mobile phase A: hex (0.5% 2 M NH₃-MeOH), mobile phase B:EtOH; flow rate: 20 mL/min; gradient: 10% B to 10% B in 11 min; wavelength: 220/254 nm; RT1(min): 6.92; RT2(min): 8.68; sample solvent: EtOH:DCM=1:1; injection volume: 0.3 mL; number of runs: 7) to afford (S)-4-cyclopropyl-3-(tetrahydrofuran-3-yl)-N-(2-(trifluoromethyl)pyridin-4-yl)isothiazole-5-carboxamide (I-204, 30.9 mg, 12.15%) as a white solid. LCMS (ES, m/z): [M+H]⁺=384; ¹H NMR (400 MHz, DMSO-d₆) δ 0.52-0.57 (m, 2H), 0.91-0.98 (m, 2H), 1.92-1.99 (m, 1H), 2.19-2.25 (m, 1H), 2.30-2.37 (m, 1H), 3.80-3.95 (m, 4H), 4.14 (t, J=7.4 Hz, 1H), 7.91 (dd, J=5.4, 2.0 Hz, 1H), 8.18 (d, J=2.0 Hz, 1H), 8.71 (d, J=5.4 Hz, 1H), 11.27 (s, 1H).

### EXAMPLE 289—SYNTHESIS OF (R)-4-CYCLOPROPYL-3-(TETRAHYDROFURAN-3-YL)-N-(2-(TRIFLUOROMETHYL)PYRIDIN-4-YL)ISOTHIAZOLE-5-CARBOXAMIDE (I-205)

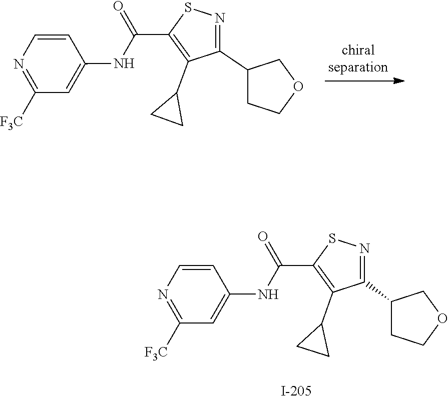 [SMILES: O=C(Nc1ccnc(C(F)(F)F)c1)c1snc(C2CCOC2)c1C1CC1 ; O=C(Nc1ccnc(C(F)(F)F)c1)c1snc([C@H]2CCOC2)c1C1CC1]

4-Cyclopropyl-3-(tetrahydrofuran-3-yl)-N-(2-(trifluoromethyl)pyridin-4-yl)isothiazole-5-carboxamide (170 mg) was purified by prep-chiral-HPLC with the following conditions: (column: CHIRALPAK IF, 2*25 cm, 5 μm; mobile phase A: hex (0.5% 2 M NH₃-MeOH), mobile phase B:EtOH; flow rate: 20 mL/min; gradient: 10% B to 10% B in 11 min; wavelength: 220/254 nm; RT1(min): 6.92; RT2(min): 8.68; sample solvent: EtOH:DCM=1:1; injection volume: 0.3 mL; number of runs: 7) to afford (R)-4-cyclopropyl-3-(tetrahydrofuran-3-yl)-N-(2-(trifluoromethyl)pyridin-4-yl)isothiazole-5-carboxamide (I-205, 49.2 mg, 19.25%) as a white solid. LCMS (ES, m/z): [M+H]⁺=384; ¹H NMR (400 MHz, DMSO-d₆) δ 0.52-0.57 (m, 2H), 0.91-0.98 (m, 2H), 1.92-1.99 (m, 1H), 2.19-2.25 (m, 1H), 2.30-2.37 (m, 1H), 3.80-3.95 (m, 4H), 4.14 (t, J=7.4 Hz, 1H), 7.91 (dd, J=5.4, 2.0 Hz, 1H), 8.18 (d, J=2.0 Hz, 1H), 8.71 (d, J=5.4 Hz, 1H), 11.27 (s, 1H).

### EXAMPLE 290—SYNTHESIS OF N-(5-CYANO-6-(2H-1,2,3-TRIAZOL-2-YL)PYRIDIN-3-YL)-4-CYCLOPROPYL-3-(2-METHYL-2H-INDAZOL-4-YL)ISOTHIAZOLE-5-CARBOXAMIDE (1-206)

To a stirred mixture of methyl 4-cyclopropyl-3-(2-methyl-2H-indazol-4-yl)isothiazole-5-carboxylate (100.00 mg, 0.319 mmol, 1 equiv) and 5-amino-2-(2H-1,2,3-triazol-2-yl)nicotinonitrile (71.29 mg, 0.383 mmol, 1.2 equiv) in THF (3 mL) was added t-BuOK (107.42 mg, 0.957 mmol, 3 equiv) at room temperature. The resulting mixture was stirred for 10 min at room temperature. The resulting mixture was diluted with water (10 mL) and extracted with EtOAc (3×15 mL). The combined organic layers were washed with brine (1×40 mL) and dried over anhydrous Na₂SO₄. After filtration, the filtrate was concentrated under reduced pressure. The crude product was purified by prep-HPLC with the following conditions: column: XBridge Shield RP18 OBD column: 30*150 mm, 5 μm; mobile phase A: water (10 mmol/L NH₄HCO₃+0.05% NH₃H₂O), mobile phase B: ACN; flow rate: 60 mL/min; gradient: 30% B to 53% B in 7 min; wavelength: 254 nm/220 nm; RT1(min): 6.27, to afford N-(5-cyano-6-(2H-1,2,3-triazol-2-yl)pyridin-3-yl)-4-cyclopropyl-3-(2-methyl-2H-indazol-4-yl)isothiazole-5-carboxamide (I-206, 22.9 mg, 15.23%) as an off-white solid. LCMS (ES, m/z): [M+H]⁺=468; ¹H NMR (400 MHz, DMSO-d₆) δ 0.34-0.38 (m, 2H), 0.81-1.01 (m, 2H), 2.19-2.35 (m, 1H), 4.22 (s, 3H), 7.41 (dd, J=7.2, 8.8 Hz, 1H), 7.67 (d, J=7.2 Hz, 1H), 7.74 (d, J=8.8 Hz, 1H), 8.32 (s, 2H), 8.56 (s, 1H), 8.86 (d, J=2.4 Hz, 1H), 9.10 (d, J=2.4 Hz, 1H), 11.42 (s, 1H).

### EXAMPLE 291—SYNTHESIS OF 4-CYCLOPROPYL-3-(2-METHYL-2H-BENZO[D][1,2,3]TRIAZOL-4-YL)-N-(2-(TRIFLUOROMETHYL)PYRIDIN-4-YL) ISOTHIAZOLE-5-CARBOXAMIDE (I-207)

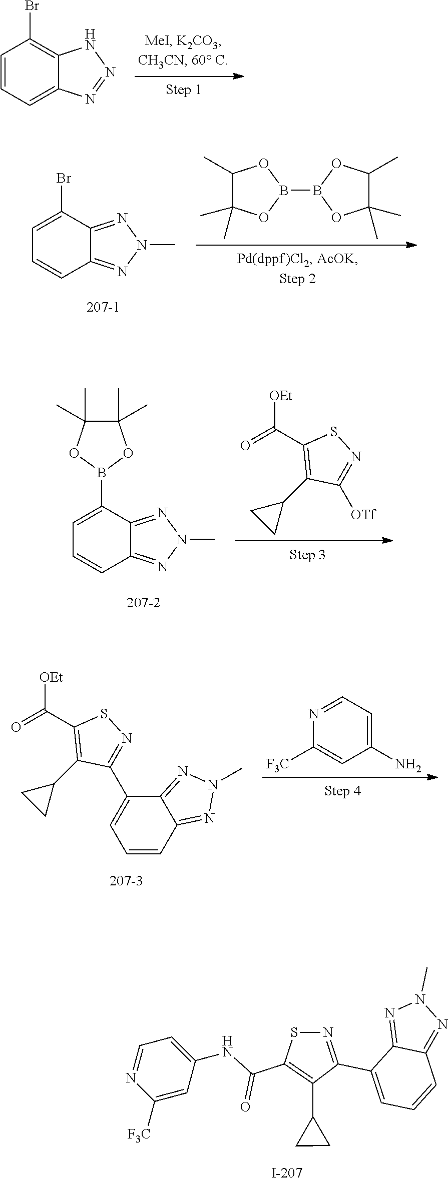

Step 1: Synthesis of 207-1: A solution of 7-bromo-1H-benzo[d][1,2,3]triazole (1.5 g, 7.575 mmol, 1 equiv), MeI (3 g, 21.136 mmol, 2.79 equiv) and K₂CO₃ (3.2 g, 23.154 mmol, 3.06 equiv) in MeCN (20 mL) was stirred overnight at 60° C. under a nitrogen atmosphere. The resulting mixture was diluted with water (10 mL). The resulting mixture was extracted with EtOAc (3×100 mL). The combined organic layers were washed with brine (1×5 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 36% to 65% gradient in 10 min; detector: UV 254 nm. This resulted in 4-bromo-2-methyl-2H-benzo[d][1,2,3]triazole (207-1, 330 mg, 20.54%) as an off-white solid.

Step 2: Synthesis of 207-2: A mixture of 4-bromo-2-methyl-2H-benzo[d][1,2,3]triazole (200 mg, 0.943 mmol, 1 equiv), 4,4,4′,4′,5,5′-hexamethyl-2,2′-bi(1,3,2-dioxaborolane) (479 mg, 1.886 mmol, 2.00 equiv), Pd(dppf)Cl₂·CH₂Cl₂ (138 mg, 0.169 mmol, 0.18 equiv), and AcOK (260 mg, 2.649 mmol, 2.81 equiv) in dioxane (8 mL) was stirred overnight at 90° C. under a nitrogen atmosphere. The resulting mixture was diluted with water (7 mL). The resulting mixture was extracted with EtOAc (3×10 mL). The combined organic layers were washed with brine (1×7 mL) and dried over anhydrous Na₂SO₄. After filtration, the filtrate was concentrated under reduced pressure. The residue was purified by prep-TLC (PE/EA 5:1) to afford 2-methyl-4-(4,4,5,5-tetramethyl-1,3,2-dioxaborolan-2-yl)-2H-benzo[d][1,2,3]triazole (207-2, 150 mg, 61.38%) as an off-white solid.

Step 3: Synthesis of 207-3: A mixture of 2-methyl-4-(4,4,5,5-tetramethyl-1,3,2-dioxaborolan-2-yl)-2H-benzo[d][1,2,3]triazole (140 mg, 0.540 mmol, I equiv), ethyl 4-cyclopropyl-3-(((trifluoromethyl)sulfonyl)oxy)isothiazole-5-carboxylate (182 mg, 0.527 mmol, 0.98 equiv), XPhos (41 mg, 0.086 mmol, 0.16 equiv), XPhos Pd G₃ (82 mg, 0.097 mmol, 0.18 equiv), and K₃PO₄ (349 mg, 1.644 mmol, 3.04 equiv) in dioxane (7 mL) was stirred overnight at 60° C. under a nitrogen atmosphere. The resulting mixture was diluted with water (10 mL). The resulting mixture was extracted with EtOAc (3×10 mL). The combined organic layers were washed with brine (1×10 mL) and dried over anhydrous Na₂SO₄. After filtration, the filtrate was concentrated under reduced pressure. The residue was purified by prep-TLC (PE/EA 5:1) to afford ethyl 4-cyclopropyl-3-(2-methyl-2H-benzo[d][1,2,3]triazol-4-yl)isothiazole-5-carboxylate (207-3, 130 mg, 73.27%) as a off-white solid.

Step 4: Synthesis of 4-cyclopropyl-3-(2-methyl-2H-benzo[d][1,2,3]triazol-4-yl)-N-(2-(trifluoromethyl)pyridin-4-yl)isothiazole-5-carboxamide (1-207): A solution of ethyl 4-cyclopropyl-3-(2-methyl-2H-benzo[d][1,2,3]triazol-4-yl)isothiazole-5-carboxylate (70 mg, 0.432 mmol, 1.01 equiv) in THF (5 mL) was stirred for 10 min at 0° C. under an air atmosphere. To the above mixture was added t-BuOK (1.1 mL, 0.010 mmol, 0.02 equiv) dropwise over 1 min at 0° C. The resulting mixture was stirred for an additional 30 min at 0° C. The aqueous layer was extracted with EtOAc (1×5 mL). The residue was purified by silica gel column chromatography and eluted with PE/EA (6:1) to afford a crude product (80 mg). The crude product (80 mg) was purified by prep-HPLC with the following conditions: (column: XBridge Prep OBD C18 column: 30*150 mm, 5 m; mobile phase A: water (10 mmol/L NH₄HCO₃+0.05% NH₃H₂O), mobile phase B: ACN; flow rate: 60 mL/min; gradient: 39% B to 64% B in 7 min; wavelength: 254 nm; RT1(min): 7.67; number of runs: 2) to afford 4-cyclopropyl-3-(2-methyl-2H-benzo[d][1,2,3]triazol-4-yl)-N-(2-(trifluoromethyl)pyridin-4-yl)isothiazole-5-carboxamide (1-207, 12.9 mg, 6.77%) as a white solid. LCMS (ES, m/z): [M+1]⁺=445; ¹H NMR (400 MHz, DMSO-d₆) δ 0.22 (s, 2H), 0.35-0.26 (m, 2H), 2.05-1.94 (m, 1H), 4.31 (s, 3H), 7.56-7.64 (dd, J=8.4, 7.0 Hz, 2H), 7.79-7.74 (m, 1H), 7.86 (dd, J=8.4, 1.2 Hz, 1H), 8.03 (d, J=1.8 Hz, 1H), 8.51 (d, J=5.6 Hz, 1H), 11.21 (s, 1H).

### EXAMPLE 292—SYNTHESIS OF 4-CYCLOPROPYL-3-(2-METHYL-2H-INDAZOL-7-YL)-N-(2-(TRIFLUOROMETHYL)PYRIDIN-4-YL)ISOTHIAZOLE-5-CARBOXAMIDE (I-208)

Step 1: Synthesis of 208-1: A mixture of ethyl 4-cyclopropyl-3-(((trifluoromethyl)sulfonyl)oxy)isothiazole-5-carboxylate (150.00 mg, 0.434 mmol, 1 equiv), 2,7-dimethyl-2H-indazole (134.56 mg, 0.521 mmol, 1.2 equiv), XPhos (51.77 mg, 0.108 mmol, 0.25 equiv), XPhos Pd G3 (73.54 mg, 0.087 mmol, 0.2 equiv) and K₃PO₄ (276.62 mg, 1.302 mmol, 3 equiv) in dioxane (4 mL) was stirred for 2 h at 60° C. under nitrogen atmosphere. The resulting mixture was diluted with water (10 mL) and extracted with EtOAc (3×15 mL). The combined organic layers were washed with brine (1×40 mL) and dried over anhydrous Na₂SO₄. After filtration, the filtrate was concentrated under reduced pressure. The residue was purified by silica gel column chromatography and eluted with PE/EA (1:1) to afford ethyl 4-cyclopropyl-3-(2-methyl-2H-indazol-7-yl)isothiazole-5-carboxylate (208-1, 100.00 mg, 68.27%) as an off-white solid.

Step 2: Synthesis of 4-cyclopropyl-3-(2-methyl-2H-indazol-7-yl)-N-(2-(trifluoromethyl)pyridin-4-yl)isothiazole-5-carboxamide (I-208): To a stirred mixture of ethyl 4-cyclopropyl-3-(2-methyl-2H-indazol-7-yl)isothiazole-5-carboxylate (100 mg, 0.305 mmol, 1 equiv) and 2-(trifluoromethyl)pyridin-4-amine (54.47 mg, 0.336 mmol, 1.1 equiv) in THF (3 mL) was added t-BuOK (143.95 mg, 1.284 mmol, 3 equiv) at room temperature. The resulting mixture was stirred for 30 min at room temperature. The resulting mixture was diluted with water (10 mL) and extracted with EtOAc (3×15 mL). The combined organic layers were washed with brine (1×40 mL) and dried over anhydrous Na₂SO₄. After filtration, the filtrate was concentrated under reduced pressure. The crude product was purified by prep-HPLC with the following conditions: column: Xbridge Prep OBD C18 column, 30*150 mm, 5 μm; mobile phase A: water (10 mmol/L NH₄HCO₃+0.05% NH₃H₂O), mobile phase B: I; flow rate: 60 mL/min; gradient: 39% B to 64% B in 7 min; wavelength: 220 nm; RT1(min): 7.58, to afford 4-cyclopropyl-3-(2-methyl-2H-indazol-7-yl)-N-(2-(trifluoromethyl)pyridine-4-yl)isothiazole-5-carboxamide (I-208, 5.3 mg, 2.69%) as an off-white solid. LCMS (ES, m/z): [M+H]⁺=444; ¹H NMR (400 MHz, DMSO-d₆) δ 0.23-0.24 (m, 2H), 0.45-0.47 (m, 2H), 2.17-2.20 (m, 1H), 4.17 (s, 3H), 7.17 (t, J=7.6 Hz, 1H), 7.33 (d, J=6.8 Hz, 1H), 7.84 (d, J=8.2 Hz, 1H), 7.98 (d, J=4.8 Hz, 1H), 8.24 (s, 1H), 8.45 (s, 1H), 8.71 (d, J=5.2 Hz, 1H), 11.42 (s, 1H).

### EXAMPLE 293—SYNTHESIS OF 4-CYCLOPROPYL-3-(2-METHYL-2H-INDAZOL-4-YL)-N-(4-(TRIFLUOROMETHYL)PYRIDIN-2-YL)ISOTHIAZOLE-5-CARBOXAMIDE (I-209)

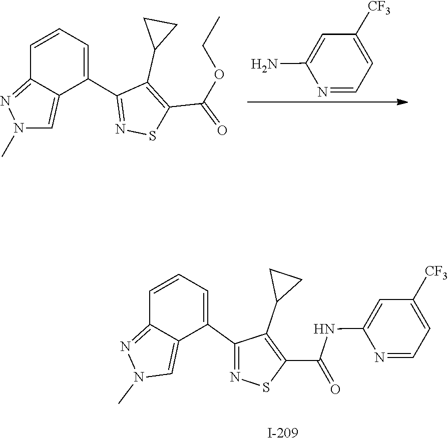

To a stirred solution of ethyl 4-cyclopropyl-3-(2-methylindazol-4-yl)-1,2-thiazole-5-carboxylate (100 mg, 0.305 mmol, 1 equiv) and 4-(trifluoromethyl)pyridine-2-amine (55 mg, 0.339 mmol, 1.11 equiv) in THF (2 mL) was added t-BuOK (103 mg, 0.915 mmol, 3 equiv) at 0° C. under a nitrogen atmosphere. The resulting mixture was stirred for 1 h at room temperature under a nitrogen atmosphere. The resulting mixture was diluted with water (10 mL). The resulting mixture was extracted with EtOAc (3×10 mL). The combined organic layers were washed with brine (1×30 mL) and dried over anhydrous Na₂SO₄. After filtration, the filtrate was concentrated under reduced pressure. The crude product was purified by prep-HPLC with the following conditions: column: Xbridge Prep OBD C18 column, 30*150 mm, 5 μm; mobile phase A: water (10 mmol/L NH₄HCO₃+0.05% NH₃H₂O), mobile phase B: I; flow rate: 60 mL/min; gradient: 44% B to 69% B in 7 min; wavelength: 220 nm; RT1(min): 7.52 to afford 4-cyclopropyl-3-(2-methylindazol-4-yl)-N-[4-(trifluoromethyl)pyridine-2-yl]-1,2-thiazole-5-carboxamide (I-209, 38.2 mg, 27.98%) as an off-white solid. LCMS (ES, m/z): [M+H]⁺=444; ¹H NMR (400 MHz, DMSO-d₆) δ 0.30-0.36 (m, 2H), 0.74-0.89 (m, 2H), 2.27-2.36 (m, 1H), 4.21 (s, 3H), 7.50-7.27 (dd, J=0.8, 7.2 Hz, 1H), 7.55-7.65 (m, 2H), 7.73 (d, J=8.8 Hz, 1H), 8.51 (s, 1H), 8.54 (s, 1H), 8.71 (d, J=5.2 Hz, 1H), 11.51 (s, 1H).

### EXAMPLE 294—SYNTHESIS OF 4-CYCLOPROPYL-3-(PYRAZOLO[1,5-A]PYRIDIN-4-YL)-N-(2-(TRIFLUOROMETHYL)PYRIDIN-4-YL)ISOTHIAZOLE-5-CARBOXAMIDE (I-210)

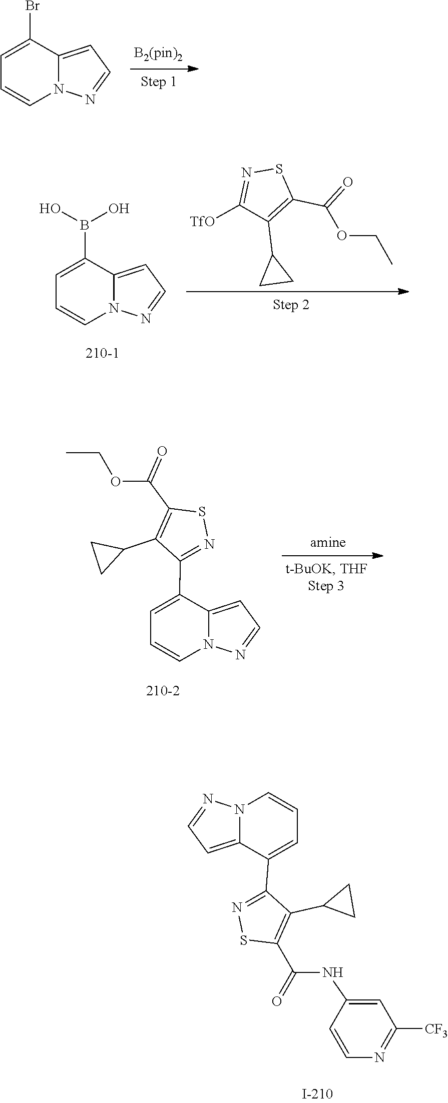 [SMILES: Brc1cccn2nccc12 ; OB(O)c1cccn2nccc12 ; CCOC(=O)c1snc(OS(=O)(=O)C(F)(F)F)c1C1CC1 ; CCOC(=O)c1snc(-c2cccn3nccc23)c1C1CC1 ; O=C(Nc1ccnc(C(F)(F)F)c1)c1snc(-c2cccn3nccc23)c1C1CC1]

Step 1: Synthesis of 210-1: A solution of 4-bromopyrazolo[1,5-a]pyridine (600 mg, 3.045 mmol, 1 equiv), bis(pinacolato)diboron (1.5 g, 5.907 mmol, 1.94 equiv), Pd(dppf)Cl₂·CH₂Cl₂ (372 mg, 0.457 mmol, 0.15 equiv) and AcOK (600 mg, 6.114 mmol, 2.01 equiv) in 1,4-dioxane (20 mL) was stirred for 2 h at 60° C. under a nitrogen atmosphere. The resulting mixture was diluted with water (10 mL). The resulting mixture was extracted with EtOAc (3×50 mL). The combined organic layers were washed with brine (1×5 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water; 50% to 70% gradient in 10 min; detector: UV 254 nm. This resulted in pyrazolo[1,5-a]pyridine-4-ylboronic acid (210-1, 280 mg, 56.77%) as a light yellow oil.

Step 2: Synthesis of 210-2: A solution of ethyl 4-cyclopropyl-3-(trifluoromethanesulfonyloxy)-1,2-thiazole-5-carboxylate (150 mg, 0.434 mmol, 1 equiv), 4-acetamidophenylboronic acid (93 mg, 0.520 mmol, 1.20 equiv), Xphos (41 mg, 0.086 mmol, 0.20 equiv), Xphos Pd G₃ (73 mg, 0.086 mmol, 0.20 equiv) and K₃PO₄ (276 mg, 1.300 mmol, 2.99 equiv) in 1,4-dioxane (10 mL) was stirred overnight at 60° C. under a nitrogen atmosphere. The resulting mixture was diluted with water (10 mL). The resulting mixture was extracted with EtOAc (3×50 mL). The combined organic layers were washed with brine (1×7 mL) and dried over anhydrous Na₂SO₄. After filtration, the filtrate was concentrated under reduced pressure. The crude product (80 mg) was purified by prep-HPLC with the following conditions (column: Xbridge Prep OBD C18 column, 30*150 mm, 5 m; mobile phase A: water (10 mmol/L NH₄HCO₃+0.1% NH₃·H₂O), mobile phase B: I; flow rate: 60 mL/min; gradient: 36% B to 65% B in 8 min, 65% B; wavelength: 220 nm; RT1(min): 7.47; number of runs: 1) to afford ethyl 4-cyclopropyl-3-(4-acetamidophenyl)-1,2-thiazole-5-carboxylate (210-2, 100 mg, 69.61%) as an off-white solid.

Step 3: Synthesis of 4-cyclopropyl-3-(pyrazolo[1,5-a]pyridine-4-yl)-N-(2-(trifluoromethyl)pyridine-4-yl)isothiazole-5-carboxamide (I-210): A solution of ethyl 4-cyclopropyl-3-(pyrazolo[1,5-a]pyridin-4-yl)isothiazole-5-carboxylate (65 mg, 0.207 mmol, 1 equiv) in THF (3 mL) was treated with 2-(trifluoromethyl)pyridine-4-amine (130 mg, 0.802 mmol, 3.87 equiv) for 15 min at 0° C. under a nitrogen atmosphere followed by the addition of t-BuOK (60 mg, 0.535 mmol, 2.58 equiv) at 0° C. The resulting mixture was stirred for 15 h at 0° C. under a nitrogen atmosphere. The mixture was acidified to pH 6 with conc. HCl. The resulting mixture was extracted with EtOAc (3×20 mL). The combined organic layers were washed with brine (1×3 mL) and dried over anhydrous Na₂SO₄. After filtration, the filtrate was concentrated under reduced pressure. The crude product (80 mg) was purified by prep-HPLC with the following conditions (column: Xbridge Prep OBD C18 column, 30*150 mm, 5 μm; mobile phase A: water (10 mmol/L NH₄HCO₃+0.05% NH₃H₂O), mobile phase B: I; flow rate: 60 mL/min; gradient: 39% B to 64% B in 7 min; wavelength: 254 nm; RT1(min): 7.67; number of runs: 2) to afford 4-cyclopropyl-3-{pyrazolo[1,5-a]pyridine-4-yl}-N-[2-(trifluoromethyl)pyridine-4-yl]-1,2-thiazole-5-carboxamide (1-210, 9.7 mg, 10.84%) as an off-white solid. LCMS (ES, m/z): [M+1]=430; ¹H NMR (400 MHz, DMSO-d₆) δ 0.32 (h, J=4.4 Hz, 2H), 0.78 (h, J=4.4 Hz, 2H), 2.19 (td, J=8.6, 4.2 Hz, 1H), 6.81 (d, J=2.4 Hz, 1H), 7.07 (t, J=7.0 Hz, 1H), 7.68 (d, J=7.0 Hz, 1H), 7.95 (dd, J=5.6, 2.0 Hz, 1H), 8.10 (d, J=2.2 Hz, 1H), 8.23 (d, J=2.0 Hz, 1H), 8.73 (d, J=5.6 Hz, 1H), 8.84 (d, J=6, 8 Hz, 1H), 11.32 (s, 1H).

### EXAMPLE 295—SYNTHESIS OF 4-CYCLOPROPYL-N-(2-METHYLPYRIDIN-4-YL)-3-(PYRAZOLO[1,5-A]PYRIDIN-4-YL)ISOTHIAZOLE-5-CARBOXAMIDE (1-211)

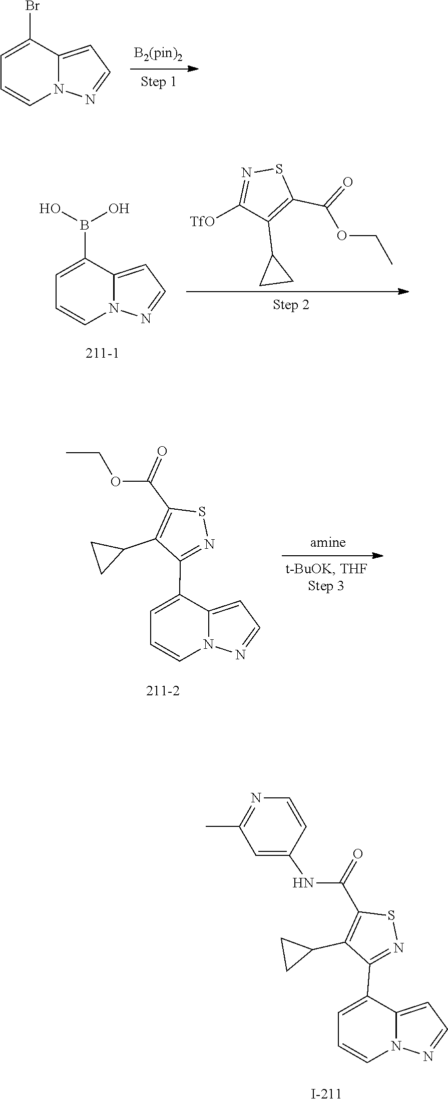 [SMILES: Brc1cccn2nccc12 ; OB(O)c1cccn2nccc12 ; CCOC(=O)c1snc(OS(=O)(=O)C(F)(F)F)c1C1CC1 ; CCOC(=O)c1snc(-c2cccn3nccc23)c1C1CC1 ; Cc1cc(NC(=O)c2snc(-c3cccn4nccc34)c2C2CC2)ccn1]

Step 1: Synthesis of 211-1: A solution of 4-bromopyrazolo[1,5-a]pyridine (600 mg, 3.045 mmol, I equiv), bis(pinacolato)diboron (1.5 g, 5.907 mmol, 1.94 equiv), Pd(dppf)Cl₂·CH₂Cl₂(372 mg, 0.457 mmol, 0.15 equiv) and AcOK (600 mg, 6.114 mmol, 2.01 equiv) in 1,4-dioxane (20 mL) was stirred for 2 h at 60° C. under a nitrogen atmosphere. The resulting mixture was diluted with water (10 mL). The resulting mixture was extracted with EtOAc (3×50 mL). The combined organic layers were washed with brine (1×5 mL) and dried over anhydrous Na₂SO₄. After filtration, the filtrate was concentrated under reduced pressure. The residue was purified by reversed-phase flash chromatography with the following conditions: column: C18 silica gel; mobile phase: MeCN in water, 45% to 66% gradient in 10 min; detector: UV 254 nm. This resulted in pyrazolo[1,5-a]pyridine-4-ylboronic acid (211-1, 280 mg, 56.77%) as a light yellow oil.

Step 2: Synthesis of 211-2: A solution of ethyl 4-cyclopropyl-3-(trifluoromethanesulfonyloxy)-1,2-thiazole-5-carboxylate (150 mg, 0.434 mmol, 1 equiv), 4-acetamidophenylboronic acid (93 mg, 0.520 mmol, 1.20 equiv), Xphos (41 mg, 0.086 mmol, 0.20 equiv), Xphos Pd G₃ (73 mg, 0.086 mmol, 0.20 equiv) and K₃PO₄ (276 mg, 1.300 mmol, 2.99 equiv) in 1,4-dioxane (10 mL) was stirred for overnight at 60° C. under nitrogen a atmosphere. The resulting mixture was diluted with water (10 mL). The resulting mixture was extracted with EtOAc (3×50 mL). The combined organic layers were washed with brine (1×5 mL) and dried over anhydrous Na₂SO₄. After filtration, the filtrate was concentrated under reduced pressure. This resulted in ethyl 4-cyclopropyl-3-(4-acetamidophenyl)-1,2-thiazole-5-carboxylate (211-2, 100 mg, 69.61%) as an off-white solid.

Step 3: Synthesis of 4-cyclopropyl-N-(2-methylpyridin-4-yl)-3-(pyrazolo[1,5-a]pyridine-4-yl)isothiazole-5-carboxamide (I-211): A solution of ethyl 4-cyclopropyl-3-{pyrazolo[1,5-a]pyridine-4-yl}-1,2-thiazole-5-carboxylate (70 mg, 0.223 mmol, I equiv) in THF (3 mL) was treated with 2-methylpyridin-4-amine (140 mg, 1.295 mmol, 5.80 equiv) for 15 min at 0° C. under a nitrogen atmosphere followed by the addition of t-BuOK (70 mg, 0.624 mmol, 2.79 equiv) at 0° C. The resulting mixture was stirred for 15 h at 0° C. under a nitrogen atmosphere. The mixture was treated with conc. HCl to a pH of 6. The resulting mixture was extracted with EtOAc (3×20 mL). The combined organic layers were washed with brine (1×3 mL) and dried over anhydrous Na₂SO₄. After filtration, the filtrate was concentrated under reduced pressure. The crude product (80 mg) was purified by prep-HPLC with the following conditions (column: Xbridge Prep OBD C18 column, 30*150 mm, 5 μm; mobile phase A: water (10 mmol/L NH₄HCO₃+0.1% NH₃·H₂O), mobile phase B: I; flow rate: 60 mL/min; gradient: 36% B to 65% B in 8 min, 65% B; wavelength: 220 nm; RT1(min): 7.47; number of runs: 1) to afford 4-cyclopropyl-N-(2-methylpyridin-4-yl)-3-{pyrazolo[1,5-a]pyridine-4-yl}-1,2-thiazole-5-carboxamide (I-211, 3.7 mg, 4.39%) as an off-white solid. LCMS (ES, m/z): [M+1]⁻=376; ¹H NMR (400 MHz, DMSO-d₆) δ 0.32 (td, J=6.2, 4.4 Hz, 2H) 0.81-0.72 (m, 2H), 2.18 (tt, J=8.6, 5.8 Hz, 1H), 2.47 (s, 3H), 6.81 (d, J=2.4 Hz, 1H), 7.06 (t, J=7.0 Hz, 1H), 7.50 (dd, J=5.6, 2.0 Hz, 1H), 7.59 (d, J=2.0 Hz, 1H), 7.66 (d, J=7.0 Hz, 1H), 8.09 (d, J=2.4 Hz, 1H), 8.40 (d, J=5.6 Hz, 1H), 8.83 (d, J=6.8 Hz, 1H), 10.94 (s, 1H).

### EXAMPLE 296—SYNTHESIS OF N-(5-CHLORO-6-(3-HYDROXYAZETIDINE-1-CARBONYL)PYRIDIN-3-YL)-4-CYCLOPROPYL-3-(PYRAZOLO[1,5-A]PYRIDIN-4-YL)ISOTHIAZOLE-5-CARBOXAMIDE (I-212)

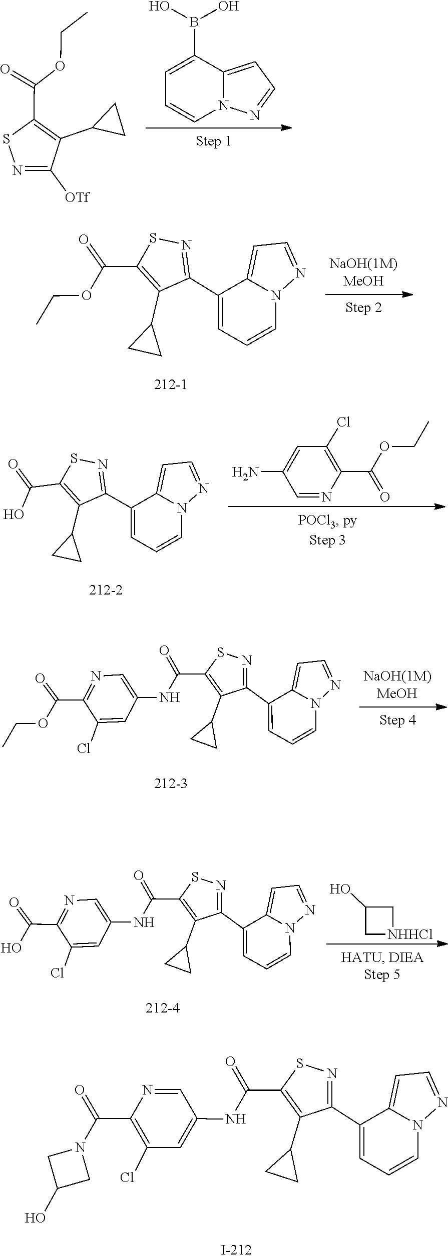

Step 1: Synthesis of 212-1: A mixture of ethyl 4-cyclopropyl-3-(trifluoromethanesulfonyloxy)-1,2-thiazole-5-carboxylate (280 mg, 0.811 mmol, 1 equiv), pyrazolo[1,5-a]pyridin-4-ylboronic acid (170 mg, 1.050 mmol, 1.29 equiv), XPhos Pd G3 (130 mg, 0.154 mmol, 0.19 equiv), and K₃PO₄ (320 mg, 1.508 mmol, 1.86 equiv) in dioxane (10 mL) was stirred for overnight at 80° C. under a nitrogen atmosphere. After completion of the reaction, the reaction mixture was quenched by the addition of water (5 mL). The aqueous layer was extracted with ethyl acetate (3×10 mL). The combined organic phase was washed with brine (20 mL), dried over anhydrous Na₂SO₄ and concentrated under reduced pressure to give the crude product, which was purified by column chromatography using 10% to 30% ethyl acetate in PE to afford the desired compound ethyl 4-cyclopropyl-3-{pyrazolo[1,5-a]pyridin-4-yl}-1,2-thiazole-5-carboxylate (212-1, 100 mg, 39.35%) as a yellow solid.

Step 2: Synthesis of 212-2: A mixture of ethyl 4-cyclopropyl-3-{pyrazolo[1,5-a]pyridin-4-yl}-1,2-thiazole-5-carboxylate (90 mg, 0.287 mmol, 1 equiv) and NaOH (72.02 mg, 1.799 mmol, 6.27 equiv) in THF (5 mL) was stirred for 2 h at room temperature under a nitrogen atmosphere. The resulting mixture was concentrated under reduced pressure. The residue was treated with conc. HCl to a pH of 6. The precipitated solids were collected by filtration and washed with water (5 ml). 4-cyclopropyl-3-{pyrazolo[1,5-a]pyridin-4-yl}-1,2-thiazole-5-carboxylic acid (212-2, 65 mg, 79.32%) was obtained as a white solid.

Step 3: Synthesis of 212-3: A mixture of 4-cyclopropyl-3-{pyrazolo[1,5-a]pyridin-4-yl}-1,2-thiazole-5-carboxylic acid (50 mg, 0.175 mmol, 1 equiv) and POCl₃ (80 mg, 0.522 mmol, 2.98 equiv) in pyridine (2 mL) was stirred overnight at 50° C. under a nitrogen atmosphere. The resulting mixture was concentrated under reduced pressure. The resulting mixture was diluted with water (10 mL). The resulting mixture was extracted with EtOAc (3×10 mL). The combined organic layers were washed with brine (20 mL) and dried over anhydrous Na₂SO₄. After filtration, the filtrate was concentrated under reduced pressure. The residue was purified by prep-TLC (CH₂Cl₂/MeOH 10:1) to afford ethyl 3-chloro-5-(4-cyclopropyl-3-{pyrazolo[1,5-a]pyridin-4-yl}-1,2-thiazole-5-amido)pyridine-2-carboxylate (212-3, 53 mg, 64.63%) as a yellow solid.

Step 4: Synthesis of 212-4: A mixture of ethyl 3-chloro-5-(4-cyclopropyl-3-{pyrazolo[1,5-a]pyridin-4-yl}-1,2-thiazole-5-amido)pyridine-2-carboxylate (50 mg, 0.107 mmol, 1 equiv) and NaOH (35 mg, 0.875 mmol, 8.19 equiv) in MeOH (2 mL)/water (0.2 mL) was stirred for 2 h at room temperature under a nitrogen atmosphere. The resulting mixture was concentrated under vacuum. The mixture was acidified to pH 6 with conc. HCl. The precipitated solids were collected by filtration to give 3-chloro-5-(4-cyclopropyl-3-{pyrazolo[1,5-a]pyridin-4-yl}-1,2-thiazole-5-amido)pyridine-2-carboxylic acid (212-4, 33 mg, 70.21%) as a white solid.

Step 5: Synthesis of N-(5-chloro-6-(3-hydroxyazetidine-1-carbonyl)pyridin-3-yl)-4-cyclopropyl-3-(pyrazolo[1,5-a]pyridin-4-yl)isothiazole-5-carboxamide (1-212): A solution of 3-chloro-5-(4-cyclopropyl-3-{pyrazolo[1,5-a]pyridin-4-yl}-1,2-thiazole-5-amido)pyridine-2-carboxylic acid (80 mg, 0.182 mmol, 1 equiv), azetidin-3-ol hydrochloride (30 mg, 0.274 mmol, 1.51 equiv), DIEA (70.52 mg, 0.546 mmol, 3 equiv) and HATU (103 mg, 0.271 mmol, 1.49 equiv) in THF (5 mL) was stirred for 2 h at room temperature under a nitrogen atmosphere. The resulting mixture was diluted with water (10 mL). The resulting mixture was extracted with EtOAc (3×20 mL). The combined organic layers were washed with brine (1×40 mL) and dried over anhydrous Na₂SO₄. After filtration, the filtrate was concentrated under reduced pressure. The crude product (100 mg) was purified by prep-HPLC with the following conditions (column: XBridge Prep OBD C18 column, 30*150 mm, 5 μm; mobile phase A: water (10 mmol/L NH₄HCO₃)+0.05% NH₃·H₂O, mobile phase B: ACN; flow rate: 60 mL/min; gradient: 16% B to 43% B over 7 min; wavelength: 254 nm/220 nm; RT1(min): 7.68) to afford N-[5-chloro-6-(3-hydroxyazetidine-1-carbonyl)pyridin-3-yl]-4-cyclopropyl-3-{pyrazolo[1,5-a]pyridin-4-yl}-1,2-thiazole-5-carboxamide (1-212, 15.2 mg, 16.77%) as an off-white solid. (ES, m/z): [M+H]⁺=495; ¹H NMR (DMSO-d₆, 400 MHz, ppm): δ 0.01-0.33 (m, 2H), 0.76-0.85 (m, 2H), 2.10-2.30 (m, 1H), 3.65-3.91 (m, 2H), 4.15-4.22 (m, 1H), 4.23-4.33 (m, 1H), 4.50-4.70 (m, 1H), 5.84 (d, J=6.4 Hz, 1H), 6.80 (d, J=2.4 Hz, 1H), 7.07 (t, J=7.2 Hz, 1H), 7.68 (d, J=7.2 Hz, 1H), 8.10 (d, J=2.4 Hz, 1H), 8.41 (d, J=2.0 Hz, 1H), 8.77-8.87 (m, 2H), 11.20 (s, 1H).

### EXAMPLE 297—MALT1 INHIBITION ASSAY

Inhibition of MALT1 activity by a small molecule was evaluated using MALT-1 Fluorogenic Peptide Cleavage Assay. The assay utilizes a quenched AMC-labelled peptide that contains the MALT-1 recognition sequence and cleavage site (LRSR). MALT-1 mediated cleavage of the peptide relieves the quenching and leads to an increase in fluorescence at excitation (342 nm) and emission (441 nm).

The following reagents were obtained commercially and used to prepare standard reagent formulations as further described below: N-2-hydroxyethylpiperazine-N-2-ethane sulfonic acid (HEPES) (1 M, pH 7.5, stored at 4° C.), sodium citrate (stored at RT), tris(2-carboxyethyl)phosphine hydrochloride (T-CEP) (500 mM, stored at −20° C.), ethylenediaminetetraacetic acid (EDTA) (500 mM, stored at RT), dimethylsulfoxide (DMSO, stored at RT), 3-[(3-cholamidopropyl)dimethylammonio]-1-propane sulphate (CHAPS) (stored at RT), dimethylsulfoxide (DMSO), DMSO (Fisher Scientific, stored at RT), Avi-tagged FL MALT-1 (Pharmaron, stored at −70° C.), Ac-LRSR-AMC Peptide (SM Biochemicals, stored at −20° C.), and ultrapure water (MILLI-Q®).

The standard reagent formulations used in this assay were prepared and stored as follows. A 1.1 M solution of sodium citrate (161.3 g, 500 mL) was prepared and stored at room temperature. A 10% (w/v) CHAPS solution (2.0 g, 20 mL) was prepared and stored at 4° C. A 500 mM HEPES solution having pH of 6.89 was prepared from 200 mL of a 1 M HEPES solution having a pH of 7.5 using concentrated hydrochloric acid and brought to a final volume of 400 mL with MILLI-Q© H₂O. The substrate, 10 mM Ac-LRSR-AMC Peptide, was prepared (10 mg, 1.370 mL DMSO) and stored at −20° C.

Compounds were plated to provide a 2% DMSO final concentration using a ProxiPlate-384 Plus F Black 384-shallow well microplate. The assay-ready plates were equilibrated to room temperature. A reaction buffer (30 mL total volume) was prepared by combining HEPES (pH 6.89, 25 mM, 1.5 mL), sodium citrate (660 mM, 18.0 mL), T-CEP (1 mM, 0.06 mL), EDTA (0.1 mM, 0.06 mL), CHAPS (0.05%, 0.15 mL), DMSO (2%, 0.6 mL), and MILLI-Q® H₂O (10.23 mL; 9.63 mL when backfilled to 2% DMSO) followed by thorough mixing. DMSO was added only when compound plates had not been DMSO-backfilled to 2%.

MALT-1 was thawed and kept on ice. Peptide substrate was thawed on the bench under ambient conditions. A MALT-1 enzyme working stock was prepared from Avi-tagged FL MALT-1 (40 nM in a prepared reagent volume of 16.5 L) and the reaction buffer (13.0 mL). MALT1 working stock (5 μL) was added to each well of the microplate. MALT-1 was pre-incubated with compounds for 30 minutes at room temperature.

Two substrate working stocks (Km and 10×Km) were prepared. The 1×Km substrate was prepared from Ac-LRSR-AMC Peptide (50 μM in a prepared reagent volume of 35.0 L) and the reaction buffer (6.965 mL). The 10×Km substrate was prepared from Ac-LRSR-AMC Peptide (280 μM in a prepared reagent volume of 196.0 μL) and the reaction buffer (6.804 mL). The reaction was initiated by the addition of substrate working stock (5 μL, 50 μM) to Km plates and the addition of substrate working stock (5 μL 280 μM) to 10×Km plates. The plates were covered and incubated on the bench at room temperature for 90 minutes.

Fluorescence intensity was determined using a CLARIOstar microplate reader (BMG LABTECH) using the optimised AMC mode. Before taking readings, a 70% gain was applied on the neutral control well. Compound data were normalised to % Inhibition and used to plot sigmoidal concentration response curves to yield various parameters including IC₅₀.

The results of the MALT1 assay are reported in Table 2 below. Compounds with an IC₅₀ less than or equal to 400 nM are designated as “A”. Compounds with an IC₅₀ greater than 400 nM and less than or equal to 1000 nM are designated as “B”. Compounds with an IC₅₀ greater than 1000 nM and less than or equal to 2500 nM are designated as “C”. Compounds with an IC₅₀ greater than 2500 nM are designated as “D”.

TABLE 2

| Compound No. | IC₅₀ |
|---|---|
| I-1 | A |
| I-2 | C |
| I-3 | A |
| I-4 | B |
| I-5 | A |
| I-6 | A |
| I-7 | A |
| I-8 | A |
| I-9 | A |
| I-10 | A |
| I-11 | A |
| I-12 | A |
| I-13 | A |
| I-14 | A |
| I-15 | A |
| I-16 | A |
| I-17 | A |
| I-18 | A |
| I-19 | A |
| I-20 | A |
| I-21 | A |
| I-22 | A |
| I-23 | A |
| I-24 | A |
| I-25 | A |
| I-26 | A |
| I-27 | A |
| I-28 | A |
| I-29 | A |
| I-30 | A |
| I-31 | A |
| I-32 | A |
| I-33 | A |
| I-34 | A |
| I-35 | A |
| I-36 | A |
| I-37 | B |
| I-38 | A |
| I-39 | A |
| I-40 | A |
| I-41 | D |
| I-42 | A |
| I-43 | D |
| I-44 | D |
| I-45 | C |
| I-46 | D |
| I-47 | B |
| I-48 | A |
| I-53 | A |
| I-54 | A |
| I-55 | A |
| I-56 | A |
| I-57 | A |
| I-58 | A |
| I-59 | D |
| I-60 | A |
| I-61 | D |
| I-62 | D |
| I-98 | D |
| I-49 | C |
| I-50 | A |
| I-51 | A |
| I-52 | D |
| I-99 | A |
| I-100 | A |
| I-101 | A |
| I-102 | A |
| I-103 | A |
| I-104 | A |
| I-105 | A |
| I-106 | A |
| I-107 | A |
| I-108 | C |
| I-109 | A |
| I-110 | B |
| I-111 | D |
| I-112 | A |
| I-113 | A |
| I-114 | A |
| I-115 | A |
| I-116 | A |
| I-117 | A |
| I-118 | A |
| I-119 | A |
| I-120 | A |
| I-121 | C |
| I-122 | D |
| I-123 | A |
| I-124 | C |
| I-125 | D |
| I-126 | A |
| I-127 | A |
| I-128 | D |
| I-129 | D |
| I-130 | D |
| I-131 | A |
| I-132 | A |
| I-133 | A |
| I-134 | D |
| I-135 | A |
| I-136 | A |
| I-137 | B |
| I-138 | A |
| I-139 | A |
| I-140 | A |
| I-141 | B |
| I-142 | A |
| I-143 | A |
| I-144 | A |
| I-145 | D |
| I-146 | A |
| I-147 | A |
| I-148 | B |
| I-149 | A |
| I-150 | C |
| I-151 | B |
| I-152 | A |
| I-153 | A |
| I-154 | A |
| I-155 | A |
| I-156 | A |
| I-157 | A |
| I-158 | A |
| I-159 | A |
| I-160 | A |
| I-161 | A |
| I-162 | A |
| I-163 | A |
| I-164 | A |
| I-165 | A |
| I-166 | A |
| I-167 | B |
| I-168 | B |
| I-169 | A |
| I-170 | A |
| I-171 | A |
| I-172 | A |
| I-173 | A |
| I-174 | A |
| I-175 | A |
| I-176 | C |
| I-177 | A |
| I-178 | A |
| I-179 | B |
| I-180 | A |
| I-181 | C |
| I-182 | A |
| I-183 | A |
| I-184 | A |
| I-185 | C |
| I-186 | A |
| I-187 | A |
| I-188 | A |
| I-189 | A |
| I-190 | A |
| I-191 | A |
| I-192 | A |
| I-193 | B |
| I-194 | A |
| I-195 | A |
| I-196 | A |
| I-197 | A |
| I-198 | A |
| I-199 | A |
| I-200 | A |
| I-201 | D |
| I-202 | D |
| I-203 | B |
| I-204 | D |
| I-205 | C |
| I-206 | A |
| I-207 | A |
| I-208 | B |
| I-209 | D |
| I-210 | A |
| I-211 | A |
| I-212 | A |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A compound represented by Formula I: 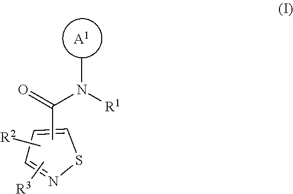
or a pharmaceutically acceptable salt thereof; wherein:
A¹ is phenyl, a 6 membered heteroaryl containing 1 or 2 nitrogen atoms, a 9-10 membered bicyclic heteroaryl containing 1, 2, or 3 heteroatoms independently selected from nitrogen and oxygen, 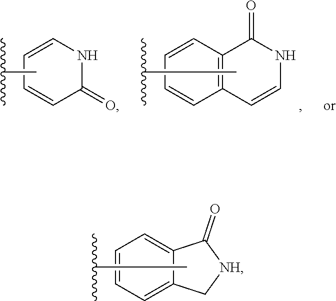
wherein the phenyl, heteroaryl, 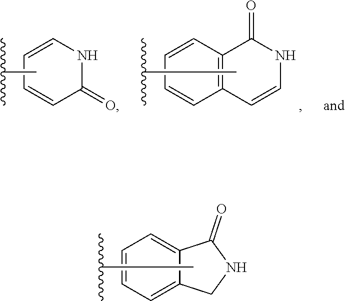
and are substituted with m occurrences of R⁵ and n occurrences of R⁶;
A² represents independently for each occurrence a 4-6 membered saturated heterocyclyl containing 1 or 2 heteroatoms independently selected from oxygen, nitrogen, and sulfur, wherein the heterocyclyl is substituted with y occurrences of R⁷;
R¹ is hydrogen or C₁₋₄ alkyl;
R² is C₁₋₆ haloalkyl, C₁₋₆ alkyl, C₃₋₇ cycloalkyl, C₁₋₆ alkoxyl, cyano, C₂₋₄ alkenyl, halogen, hydrogen, oxetanyl, tetrahydrofuranyl, or —N(R¹²)(R¹³), wherein the oxetanyl and tetrahydrofuranyl are substituted with 0 or 1 hydroxyl groups;
R³ is one of the following:
(a) phenyl, a 5-6 membered heteroaryl containing 1, 2, or 3 heteroatoms independently selected from oxygen, nitrogen, and sulfur, a 9-10 membered bicyclic heteroaryl containing 1, 2, or 3 heteroatoms independently selected from oxygen, nitrogen, and sulfur, a 3-10 membered monocyclic or bicyclic saturated or partially unsaturated heterocyclyl containing 1, 2, or 3 heteroatoms independently selected from oxygen, nitrogen, and sulfur, 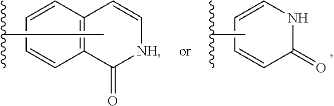
wherein the phenyl, heteroaryl, heterocyclyl, 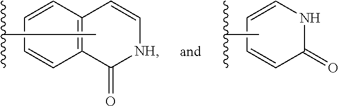
are substituted with t occurrences of R⁴; or
(b) C₂₋₆ alkyl, hydroxyl, or —N(R⁹)(R¹⁰);
R⁴ represents independently for each occurrence halo, hydroxyl, C₁₋₆ alkyl, C₁₋₆ haloalkyl, C₁₋₆hydroxyalkyl, C₁₋₆ alkoxyl, C₃₋₇ cycloalkyl, —C(O)R¹¹, —C(O)N(R⁹)(R¹⁰), cyano, —N(R¹²)(R¹³), or —N(R⁹)C(O)R¹¹;
R⁵ is a 5-6 membered heteroaryl containing 1, 2, or 3 heteroatoms independently selected from oxygen, nitrogen, and sulfur, or a 3-7 membered saturated heterocyclyl containing 1, 2, or 3 heteroatoms independently selected from oxygen, nitrogen, and sulfur, wherein the heteroaryl and heterocyclyl are substituted with q occurrences of R⁷;
R⁶ represents independently for each occurrence halo, hydroxyl, C₁₋₆ alkyl, C₁₋₆ haloalkyl, C₁₋₆hydroxyalkyl, C₁₋₆ alkoxyl, C₃₋₇ cycloalkyl, cyano, —O—C₃₋₇ cycloalkyl, —N(R⁹)(R¹⁰), —(C₀₋₄ alkylene)-C(O)R⁸, —C(O)N(R⁹)(R¹⁰), or —N(R⁹)C(O)R¹¹;
R⁷ represents independently for each occurrence halo, hydroxyl, C₁₋₆ alkyl, C₁₋₆ haloalkyl, C₁₋₆ alkoxyl, or C₃₋₇ cycloalkyl;
R⁸ is —OH, —O—(C₁₋₆ alkyl), —O—C₃₋₇ cycloalkyl, or A²;
R⁹ and R¹⁰ are independently hydrogen, C₁₋₆ alkyl, C₃₋₇ cycloalkyl, C₂₋₄ hydroxyalkyl, or —(C₂₋₆ alkylene)-(C₁₋₆ alkoxyl), or R⁹ and R¹⁰ are taken together with the nitrogen atom to which they are attached to form a 3-7 membered heterocyclic ring containing 1 nitrogen atom, wherein the heterocyclic ring is substituted with 0 or 1 groups independently selected from hydroxyl, halo, and C₁₋₆ alkyl;
R¹¹ represents independently for each occurrence C₁₋₆ alkyl or (C₀₋₅ alkylene)-C₃₋₇ cycloalkyl;
R¹² and R¹³ are independently hydrogen, C₁₋₆ alkyl, or C₃₋₅ cycloalkyl, or R¹² and R¹³ are taken together with the nitrogen atom to which they are attached to form a 3-7 membered heterocyclic ring containing 1 nitrogen atom;
m is 0 or 1; and
n, q, t, and y are independently 0, 1, or 2;
provided that when R² is hydrogen and m is 1, then R⁵ is 1,2,3-triazolyl substituted with q occurrences of R⁷.

2. The compound of claim 1, wherein the compound is a compound of Formula I.

3. The compound of claim 1 or 2, wherein R² is C₁₋₆ haloalkyl.

4. The compound of claim 1 or 2, wherein R² is —CF₃.

5. The compound of claim 1 or 2, wherein R² is C₃₋₇cycloalkyl.

6. The compound of claim 1 or 2, wherein R² is cyclopropyl.

7. The compound of claim 1 or 2, wherein R² is C₁₋₆ alkyl or C₂₋₄ alkenyl.

8. The compound of claim 1 or 2, wherein R² is halogen, C₁₋₆ alkoxyl, or cyano.

9. The compound of any one of claims 1-8, wherein the compound is a compound of Formula Ia or a pharmaceutically acceptable salt thereof:

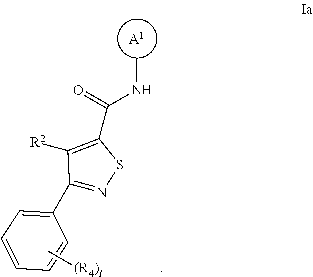

10. The compound of any one of claims 1-8, wherein the compound is a compound of Formula Ib or Ic or a pharmaceutically acceptable salt thereof:

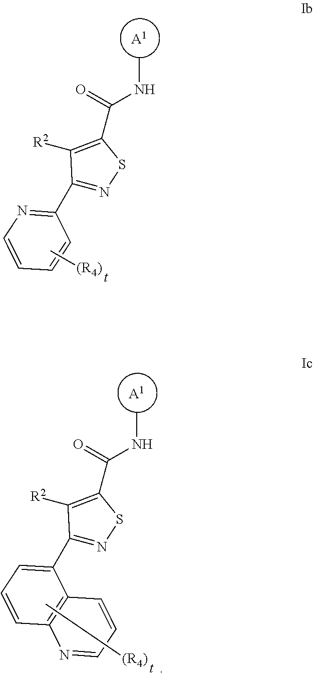

11. The compound of any one of claims 1-10, wherein A¹ is phenyl substituted with m occurrences of R⁵ and n occurrences of R⁶.

12. The compound of any one of claims 1-10, wherein A¹ is a 6 membered heteroaryl containing 1 or 2 nitrogen atoms, wherein the heteroaryl is substituted with m occurrences of R⁵ and n occurrences of R⁶.

13. The compound of any one of claims 1-10, wherein A¹ is pyridinyl substituted with m occurrences of R⁵ and n occurrences of R⁶.

14. The compound of any one of claims 1-10, wherein A¹ is a 9-10 membered bicyclic heteroaryl containing 1, 2, or 3 heteroatoms independently selected from nitrogen and oxygen, wherein the heteroaryl is substituted with m occurrences of R⁵ and n occurrences of R⁶.

15. The compound of any one of claims 1-10, wherein A¹ is or 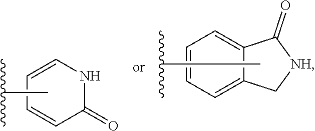
each of which is substituted with m occurrences of R⁵ and n occurrences of R⁶.

16. The compound of any one of claims 1-15, wherein n is 1.

17. The compound of any one of claims 1-15, wherein n is 0.

18. The compound of any one of claims 1-17, wherein m is 1.

19. The compound of any one of claims 1-8, wherein the compound is a compound of Formula Id or a pharmaceutically acceptable salt thereof:

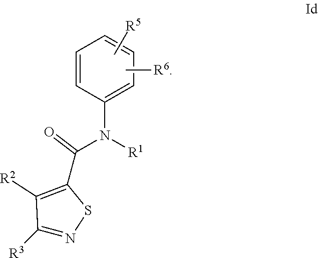

20. The compound of any one of claims 1-8, wherein the compound is a compound of Formula Ie or a pharmaceutically acceptable salt thereof:

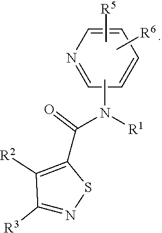

21. The compound of any one of claims 1-8, wherein the compound is a compound of Formula If or a pharmaceutically acceptable salt thereof:

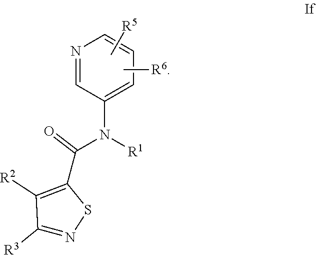

22. The compound of any one of claims 1-6 or 19-21, wherein R³ is phenyl substituted with t occurrences of R⁴.

23. The compound of any one of claims 1-6 or 19-21, wherein R³ is a 5-6 membered heteroaryl containing 1, 2, or 3 heteroatoms independently selected from oxygen, nitrogen, and sulfur, wherein the heteroaryl is substituted with t occurrences of R⁴.

24. The compound of any one of claims 1-6 or 19-21, wherein R³ is a 9-10 membered bicyclic heteroaryl containing 1, 2, or 3 heteroatoms independently selected from oxygen, nitrogen, and sulfur, wherein the heteroaryl is substituted with t occurrences of R⁴.

25. The compound of any one of claims 1-6 or 19-21, wherein R³ is a 3-10 membered monocyclic or bicyclic saturated or partially unsaturated heterocyclyl containing 1, 2, or 3 heteroatoms independently selected from oxygen, nitrogen, and sulfur, wherein the heterocyclyl is substituted with t occurrences of R⁴.

26. The compound of any one of claims 1-6 or 19-21, wherein R³ is tetrahydropyranyl, morpholinyl, piperidinyl, or piperazinyl, each of which is substituted with t occurrences of R⁴.

27. The compound of any one of claims 1-6 or 19-21, wherein R³ is 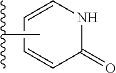
substituted with t occurrences of R⁴.

28. The compound of any one of claims 1-6 or 19-21, wherein R³ is C₃₋₄ alkyl or hydroxyl.

29. The compound of any one of claims 1-6 or 19-28, wherein R¹ is hydrogen.

30. The compound of any one of claims 1-6, wherein the compound is a compound of Formula Ig or Ih or a pharmaceutically acceptable salt thereof:

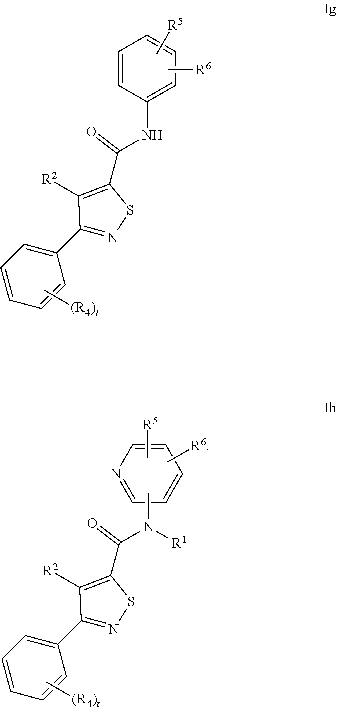

31. The compound of any one of claims 1-6, wherein the compound is a compound of Formula Ii, Ij, Ik, or Il or a pharmaceutically acceptable salt thereof:

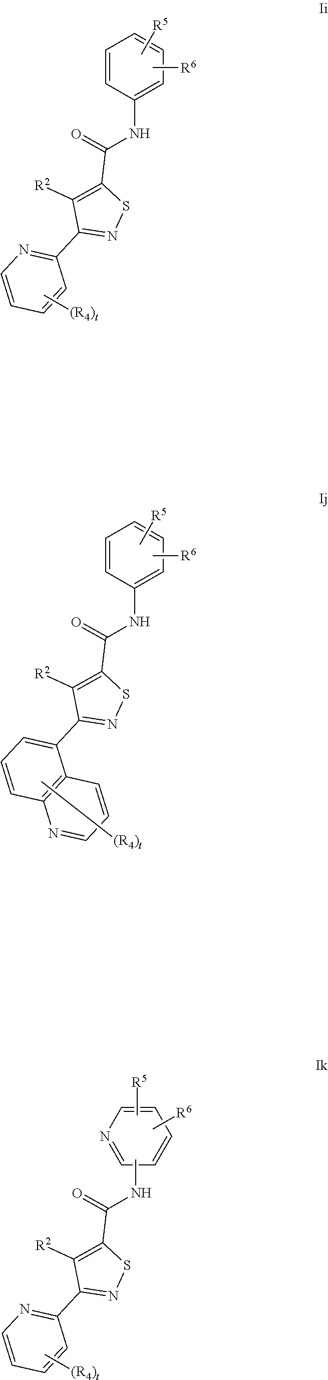

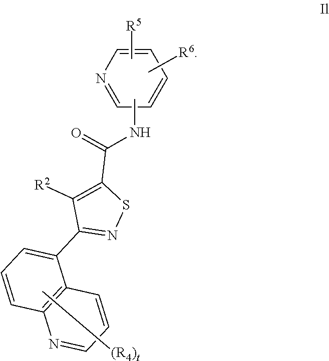

32. The compound of any one of claims 1-6, wherein the compound is a compound of Formula Im or In or a pharmaceutically acceptable salt thereof:

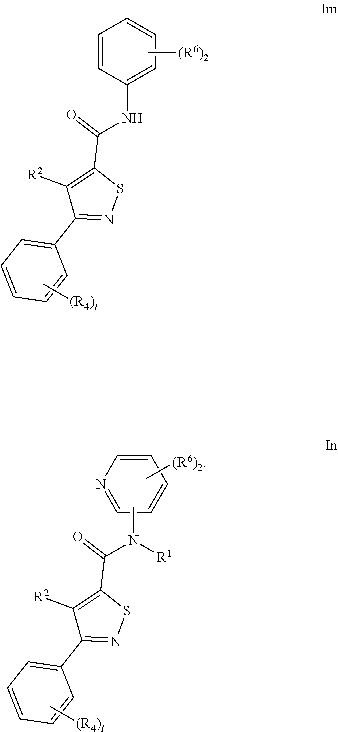

33. The compound of any one of claims 1-6, wherein the compound is a compound of Formula Io, Ip, Iq, or Ir or a pharmaceutically acceptable salt thereof:

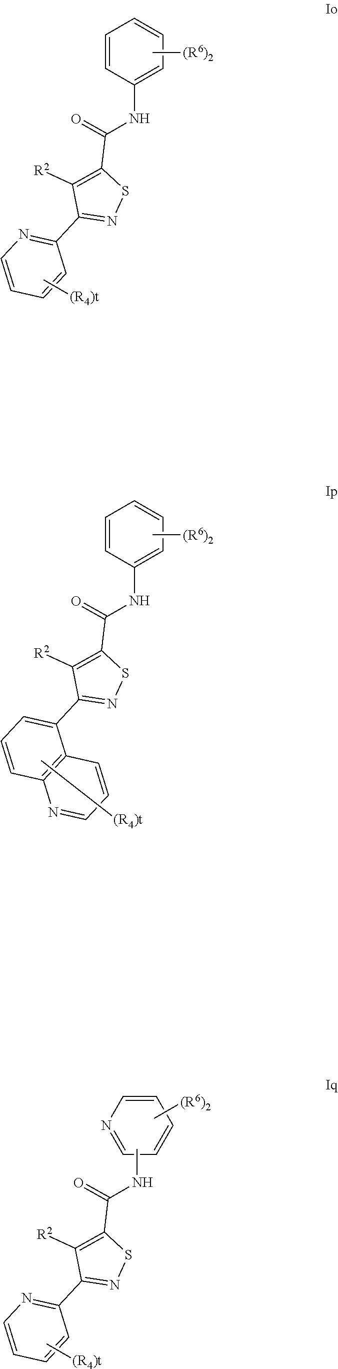

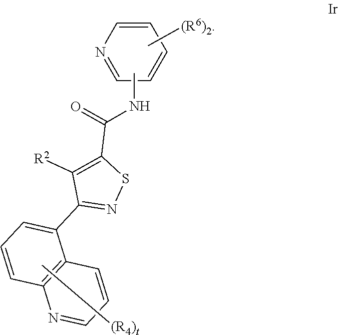

34. The compound of any one of claims 1-6, wherein the compound is a compound of Formula Is or It or a pharmaceutically acceptable salt thereof:

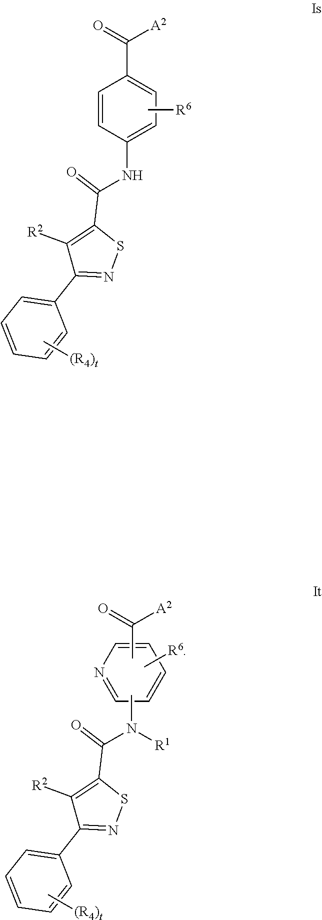

35. The compound of any one of claims 1-6, wherein the compound is a compound of Formula Iu, Iv, Iw, or Ix or a pharmaceutically acceptable salt thereof:

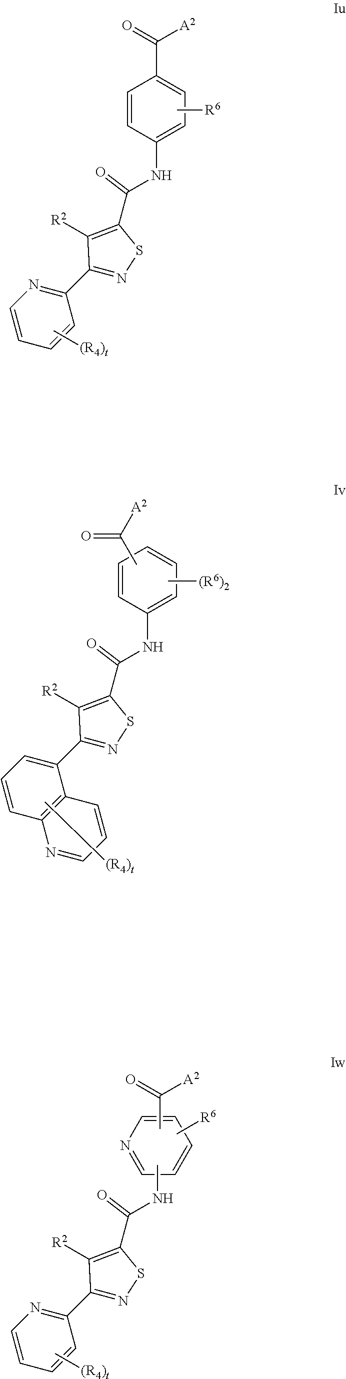

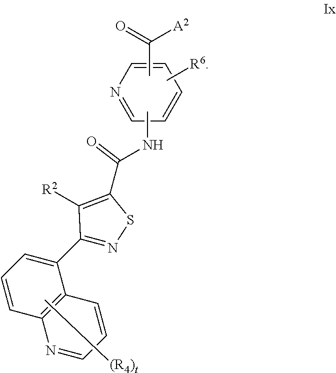

36. The compound of any one of claims 1-31, wherein R⁵ is a 5 membered heteroaryl containing 1, 2, or 3 heteroatoms independently selected from oxygen, nitrogen, and sulfur, wherein the heteroaryl is substituted with q occurrences of R⁷.

37. The compound of any one of claims 1-31, wherein R⁵ is a 1,2,3-triazolyl, pyrazolyl, oxazolyl, imidazolyl, isoxazolyl, pyrrolyl, or furanyl, each of which substituted with q occurrences of R⁷.

38. The compound of any one of claims 1-31, wherein R⁵ is 1,2,3-triazolyl substituted with q occurrences of R⁷.

39. The compound of any one of claims 1-31 or 36-38, wherein q is 0.

40. The compound of any one of claims 1-39, wherein R⁶ represents independently for each occurrence halo, C₁₋₆ alkyl, C₁₋₆ haloalkyl, cyano, or —(C₀₋₄ alkylene)-C(O)R⁸.

41. The compound of any one of claims 1-39, wherein R⁶ represents independently for each occurrence halo, cyano, or —(C₀₋₄ alkylene)-C(O)R⁸.

42. The compound of any one of claims 1-39, wherein R⁶ is chloro.

43. The compound of any one of claims 1-39, wherein R⁶ is C₁₋₆ haloalkyl.

44. The compound of any one of claims 1-39, wherein R⁶ is —CF₃.

45. The compound of any one of claims 1-39, wherein R⁶ is cyano.

46. The compound of any one of claims 1-45, wherein A² is azetidinyl substituted by hydroxyl.

47. The compound of any one of claims 1-46, wherein R⁴ represents independently for each occurrence halo, hydroxyl, C₁₋₆ alkyl, C₁₋₆ haloalkyl, C₁₋₆ hydroxyalkyl, C₁₋₆ alkoxyl, C₃₋₇ cycloalkyl, —C(O)R¹¹, —C(O)N(R⁹)(R¹⁰), or —N(R⁹)C(O)R¹¹.

48. The compound of any one of claims 1-46, wherein R⁴ represents independently for each occurrence halo, hydroxyl, C₁₋₆ alkyl, C₁₋₆ haloalkyl, or C₃₋₇ cycloalkyl.

49. The compound of any one of claims 1-46, wherein R⁴ represents independently for each occurrence halo or C₁₋₆ alkyl.

50. The compound of any one of claims 1-49, wherein t is 1.

51. The compound of any one of claims 1-46, wherein t is 0.

52. The compound of claim 1, wherein the compound is a compound of Formula Iy or a pharmaceutically acceptable salt thereof:

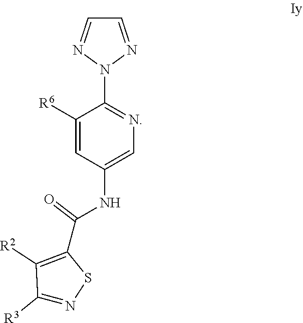

53. The compound of claim 52 wherein R³ is phenyl or 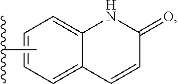
R² is C₁₋₄ haloalkyl or C₃₋₆ cycloalkyl, and R⁶ is halo or cyano.

54. The compound of claim 1, wherein the compound is a compound of Formula Iz or a pharmaceutically acceptable salt thereof:

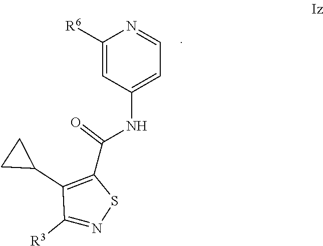

55. The compound of claim 54, wherein R³ is 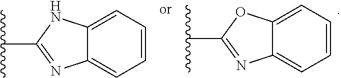
and R⁶ is C₁₋₄ alkyl or C₁₋₄ haloalkyl.

56. A compound in Table 1, or a pharmaceutically acceptable salt thereof.

57. A pharmaceutical composition comprising a compound of any one of claims 1-56 and a pharmaceutically acceptable carrier.

58. A method for treating a disease or condition mediated by MALT1, comprising administering to a subject in need thereof a therapeutically effective amount of a compound of any one of claims 1-56 to treat the disease or condition.

59. The method of claim 58, wherein said disease or condition mediated by MALT1 is a proliferative disorder.

60. The method of claim 58, wherein said disease or condition mediated by MALT1 is an inflammatory disorder.

61. The method of claim 58, wherein said disease or condition mediated by MALT1 is an autoimmune disorder.

62. The method of claim 58, wherein said disease or condition mediated by MALT1 is selected from cancer, neoplasia, chronic inflammatory disorder, acute inflammatory disorder, auto-inflammatory disorder, autoimmune disorder, fibrotic disorder, metabolic disorder, cardiovascular disorder, cerebrovascular disorder, myeloid cell-driven hyper-inflammatory response in COVID-19 infection, and a combination thereof.

63. The method of claim 58, wherein said disease or condition mediated by MALT1 is cancer.

64. The method of claim 63, wherein the cancer is lung cancer, pancreatic cancer, colorectal cancer, breast cancer, cervical cancer, prostate cancer, gastric cancer, skin cancer, liver cancer, bile duct cancer, nervous system cancer, a lymphoma, or a leukemia.

65. The method of claim 63, wherein the cancer is a lymphoma or leukemia.

66. The method of claim 63, wherein the cancer is a B-cell lymphoma or chronic myelocytic leukemia.

67. The method of claim 58, wherein said disease or condition mediated by MALT1 is Hodgkin's lymphoma, non-Hodgkin's lymphoma, Burkitt's lymphoma, diffuse large B-cell lymphoma (DLBCL), MALT lymphoma, germinal center B-cell-like diffuse large B-cell lymphoma (GCB-DLBCL), primary mediastinal B-cell lymphoma (PMBL), or activated B-cell-like diffuse large B-cell lymphoma (ABC-DLBCL).

68. The method of claim 58, wherein said disease or condition mediated by MALT1 is multiple sclerosis, ankylosing spondylitis, arthritis, osteoarthritis, juvenile arthritis, reactive arthritis, rheumatoid arthritis, psoriatic arthritis, acquired immunodeficiency syndrome (AIDS), Coeliac disease, psoriasis, chronic graft-versus-host disease, acute graft-versus-host disease, Crohn's disease, inflammatory bowel disease, multiple sclerosis, systemic lupus erythematosus, Celiac Sprue, idiopathic thrombocytopenic thrombotic purpura, myasthenia gravis, Sjogren's syndrome, scleroderma, ulcerative colitis, asthma, uveitis, rosacea, dermatitis, alopecia areata, vitiligo, arthritis, Type 1 diabetes, lupus erythematosus, systemic lupus erythenatosus, Hashirnoto's thyroiditis, myasthenia gravis, nephrotic syndrome, eosinophilia fasciitis, hyper IgE syndrome, lepromatous leprosy, sezary syndrome, idiopathic thrombocytopenia purpura, restenosis following angioplasty, a tumor, or artherosclerosis.

69. The method of claim 58, wherein said disease or condition mediated by MALT1 is allergic rhinitis, nasal inflammation, asthma, chronic obstructive pulmonary disease (COPD), bronchitis, emphysema, chronic eosinophilic pneumonia, adult respiratory distress syndrome, sinusitis, allergic conjunctivitis, idiopathic pulmonary fibrosis, atopic dermatitis, asthma, allergic rhinitis, arthritis, inflammatory bowel disease, Crohn's disease, ulcerative colitis, multiple sclerosis, endometriosis, eczema, psoriasis, rosacea, or lupus erythematosus.

70. The method of any one of claims 58-69, wherein the subject is a human.

71. A method of inhibiting the activity of MALT1, comprising contacting a MALT1 with an effective amount of a compound of any one of claims 1-56 to inhibit the activity of said MALT1.
